(19) United States
(12) Patent Application Publication
(10) Pub. No.: US 2021/0292844 A1
(43) Pub. Date: Sep. 23, 2021

(54) PROSTATE CANCER AGGRESSIVENESS BIOMARKERS

(71) Applicant: UNIVERSITY OF SOUTHERN CALIFORNIA, Los Angeles, CA (US)

(72) Inventors: Inderbir Singh Gill, Los Angeles, CA (US); Peter Jones, Los Angeles, CA (US); Gangning Liang, Los Angeles, CA (US); Kamilla Mundbjerg, Los Angeles, CA (US)

(21) Appl. No.: 17/089,491

(22) Filed: Nov. 4, 2020

Related U.S. Application Data
(63) Continuation of application No. 15/999,311, filed on Aug. 17, 2018, now abandoned.
(60) Provisional application No. 62/296,707, filed on Feb. 18, 2016.

(51) Int. Cl. C12Q 1/6886

(52) U.S. Cl. CPC C12Q 1/6886; C12Q 2600/154; C12Q 2600/112

(57) ABSTRACT

A method of identifying subjects having prostate cancer is provided. The method includes obtaining a sample from a subject, isolating DNA from the sample, determining the methylation status of the DNA, and comparing the methylation status of the DNA to one or more methylated probes selected from the following: cg00697992, cg01272707, cg01819167, cg01906055, cg02160684, cg02560085, cg03195164, cg03456213, cg04634417, cg06024295, cg11748187, cg13300630, cg13944838, cg14399930, cg15132013, cg16469740, cg17004353, cg17032646, cg18315943, cg19550524, cg20399616, cg24517686, cg25961816, cg26447413 and cg27198013. The comparison indicates whether the sample is normal, a non-aggressive prostate cancer, or an aggressive prostate cancer.

### CROSS-REFERENCE TO RELATED APPLICATIONS

This application is a Continuation Application of U.S. application Ser. No. 15/999,311 filed Aug. 17, 2018, now pending, which is a 35 USC § 371 National Stage application of International Application No. PCT/US2017/018517 filed Feb. 17, 2017, now pending; which claims the benefit under 35 USC § 119(e) to U.S. Application Ser. No. 62/296,707 filed Feb. 18, 2016, now expired. The disclosure of each of the prior applications is considered part of and is incorporated by reference in the disclosure of this application.

### INCORPORATION OF SEQUENCE LISTING

The material in the accompanying sequence listing is hereby incorporated by reference into this application. The accompanying sequence listing text file, named USC1300-1_ST25.txt, was created on Jun. 6, 2019, and is 551 KB. The file can be accessed using Microsoft Word on a computer that uses Windows OS.

### BACKGROUND OF THE INVENTION

### Field of the Invention

The present invention relates to methods of screening and diagnosing prostate cancer.

### Background Information

Prostate cancer (PC) is the most frequently diagnosed cancer and the second most common cause of cancer deaths in men in the United States. Although PC incidence rates have increased over the past 25 years, mortality rates have largely remained unchanged (www.cancer.gov). The advent of a PC diagnostic test, which measures the level of prostate specific antigen (PSA), has resulted in the discovery of earlier, smaller lesions, as well as increased the need for prognostic tools to avoid overtreatment of indolent PC¹. Localized PC is curable through radical prostatectomy or radiation therapy, albeit often followed by significant side effects, including urinary incontinence (5-20%), erectile dysfunction (30-70%), and bowel toxicity (5-10%)^{2,3}. Generally, PC is a slow-growing malignancy with decades of indolence, but takes on an aggressive form displaying rapid growth, dissemination, and lethality in a subset of cases (<20%)^{1,4}.

Predicting tumor progression is critical for clinical decision-making. PC is graded using the Gleason system, in which tumors with higher Gleason Scores (GSs) tend to be more aggressive^{5,6}. However, the relationship between individual GS of clinically-localized PCs and those that progress to metastatic disease is poorly understood⁷. The tumorigenic events during PC progression have been difficult to investigate, and the ability to characterize late stages of PC progression is lacking due to very limited availability of metastatic tissues. In addition, 60-90% of PCs are multifocal⁸, in which one prostate contains several seemingly unconnected locations of cancer growth. The development of multifocal PC is still highly debated, and two models have been described. Either one initially-transformed cancer has spread to multiple locations within the prostate (monoclonal), and/or the cancer arises independently several times in the same gland (multiple subclones)⁹⁻¹⁸. The latter option indicates the possibility that aggressive and non-aggressive cancer foci co-exist in the same prostate gland, and is supported by the finding that individual foci of multifocal PC often present with unique GSs¹⁹. Consequently, the index lesion (the cancer lesion with the largest volume or the highest GS depending on the study) may not be representative of the PC pathology²⁰, and subsequently complicates sample selection for analysis. Therefore, previous studies that have not accounted for prostate tumor multifocality, or used only the index lesion, are potentially flawed.

Currently, no curative therapies are available for metastatic PC. Thus, a central dilemma in management of clinically localized PC is whether to postpone treatment until the disease becomes more aggressive in order to minimize patient health side effects, or to treat immediately to avoid dissemination. Identifying the primary lesion that drives cancer progression is critically advantageous in order to guide treatment decisions at the time of diagnosis. In this scenario, ideally, only patients with potential aggressive PC lesion(s) should undergo surgery, while patients with exclusively clinically insignificant (non-aggressive) cancer should be monitored using active surveillance.

DNA methylation alterations occur in every cancer type, and importantly, DNA methylation levels change concordantly with tumor aggressiveness in most types of cancer²¹. Epigenetic alterations can drive tumorigenesis and determine tumor aggressiveness, and therefore, can be used for PC diagnostic purposes²², as well as to inform on therapeutic approaches^{23,53}. Although PC has been shown to harbor a great hereditary element^{24,25}, only an estimated ˜30% of these factors have presently been accounted for²⁶. Interestingly, recent studies have been able to tie a connection between genetic alterations and DNA methylation changes, which indicate that DNA methylation changes hold information about clonal evolution of PC. For example, multiple metastases within a PC patient have been shown to arise from a single precursor cancer cell, or focus, by copy number alterations (CNA) as well as by DNA methylation changes^{27,28,54}, suggesting that only one focus of a multifocal PC is responsible for the development of the metastatic lesions. Moreover, unified evolution of DNA methylation and CNAs was identified in five cases of multifocal PC with monoclonal origin and their matched lymph node metastases¹¹.

### SUMMARY OF THE INVENTION

One aspect of the present invention is directed to a method of identifying subjects having prostate cancer. The method includes obtaining a sample from a subject, isolating DNA from the sample, determining the methylation status of the DNA and comparing the methylation status of the DNA to one or more methylated probes selected from the following: cg00697992, cg01272707, cg01819167, cg01906055, cg02160684, cg02560085, cg03195164, cg03456213, cg04634417, cg06024295, cg11748187, cg13300630, cg13944838, cg14399930, cg15132013, cg16469740, cg17004353, cg17032646, cg18315943, cg19550524, cg20399616, cg24517686, cg25961816, cg26447413 and cg27198013. The methylated probe includes a sequence region that extends up to 250 base pairs upstream and downstream from the methylated probe. The comparison indicates whether the sample is normal, a non-aggressive prostate cancer, or an aggressive prostate cancer.

Examples of methylation sensitive assays that can be used to determine the DNA methylation status include but are not limited to HM450, HM850, real-time methylation sensitive PCR (MSP), MethyLight and Pyrosequencing.

In one embodiment, the sample is a biopsy sample.

In another embodiment, the biopsy is from a prostate.

In another embodiment, the biopsy is from a pelvic lymph node.

In another embodiment, two or more methylated probes are selected.

In another embodiment, the sample is selected from the following: blood, plasma and urine.

In another embodiment, the sequence region extends up to 100 base pairs upstream and downstream from the methylated probe.

In another embodiment, the sequence region extends 0 base pairs upstream and downstream from the methylated probe.

In another embodiment, five or more methylated probes are selected.

In another embodiment, fifteen or more methylated probes are selected.

Another aspect of the present invention is to provide a method of classifying prostate cancer. The method includes obtaining a sample from a subject, isolating DNA from the sample, determining the methylation status of the DNA, and comparing the methylation status of the DNA to one or more methylated probes selected from the following: cg00697992, cg01272707, cg01819167, cg01906055, cg02160684, cg02560085, cg03195164, cg03456213, cg04634417, cg06024295, cg11748187, cg13300630, cg13944838, cg14399930, cg15132013, cg16469740, cg17004353, cg17032646, cg18315943, cg19550524, cg20399616, cg24517686, cg25961816, cg26447413 and cg27198013. The methylated probe includes a sequence region that extends up to 250 base pairs upstream and downstream from the methylated probe. The comparison indicates whether the prostate cancer is non-aggressive or aggressive.

In one embodiment, the sample is a biopsy sample.

In another embodiment, the biopsy is from a prostate.

In another embodiment, the biopsy is from a pelvic lymph node.

In another embodiment, two or more methylated probes are selected.

In another embodiment, the sample is selected from the following: blood, plasma and urine.

In another embodiment, the sequence region extends up to 100 base pairs upstream and downstream from the methylated probe.

In another embodiment, the sequence region extends 0 base pairs upstream and downstream from the methylated probe.

In another embodiment, five or more methylated probes are selected.

In another embodiment, fifteen or more methylated probes are selected.

Another aspect of the present invention is directed to a method of identifying subjects having prostate cancer. The method includes obtaining a sample from a subject, isolating DNA from the sample, determining the methylation status of the DNA and comparing the methylation status of the DNA to one or more methylated probes selected from the following: cg20399616, cg12737160, cg23624008, cg09789590, cg23095615, cg14273822, cg04294888, cg12098872, cg21981270, cg21526205, cg09806262, cg01940855, cg23855505, cg18423852, cg22260952, cg21883802, cg00036011, cg26573704, cg12727795 and cg01404317. The methylated probe includes a sequence region that extends up to 250 base pairs upstream and downstream from the methylated probe. The comparison indicates whether the sample is normal or prostate cancer.

In one embodiment, the sample is a biopsy sample.

In another embodiment, the biopsy is from a prostate.

In another embodiment, the biopsy is from a pelvic lymph node.

In another embodiment, two or more methylated probes are selected.

In another embodiment, the sample is selected from the following: blood, plasma and urine.

In another embodiment, the sequence region extends up to 100 base pairs upstream and downstream from the methylated probe.

In another embodiment, the sequence region extends 0 base pairs upstream and downstream from the methylated probe.

In another embodiment, five or more methylated probes are selected.

In another embodiment, fifteen or more methylated probes are selected.

Another aspect of the present invention is to provide a method of classifying prostate cancer. The method includes obtaining a sample from a subject, isolating DNA from the sample, determining the methylation status of the DNA, and comparing the methylation status of the DNA to one or more methylated probes selected from the following: cg25729795, cg09771468, cg01386424, cg21397588, cg18595258, cg01957088, cg12950441, cg05733231, cg19155518, cg13060157, cg16201038, cg01906055, cg16473141, cg17380661, cg04145287, cg22462983, cg01419991, cg06969287, cg04634417 and cg16692973. The methylated probe includes a sequence region that extends up to 250 base pairs upstream and downstream from the methylated probe. The comparison indicates whether the prostate cancer is non-aggressive or aggressive.

In one embodiment, the sample is a biopsy sample.

In another embodiment, the biopsy is from a prostate.

In another embodiment, the biopsy is from a pelvic lymph node.

In another embodiment, two or more methylated probes are selected.

In another embodiment, the sample is selected from the following: blood, plasma and urine.

In another embodiment, the sequence region extends up to 100 base pairs upstream and downstream from the methylated probe.

In another embodiment, the sequence region extends 0 base pairs upstream and downstream from the methylated probe.

In another embodiment, five or more methylated probes are selected.

In another embodiment, fifteen or more methylated probes are selected.

In other aspects of the present invention, the methylated probes that are used in identifying subjects having prostate cancer and/or classifying prostate cancer can be one or more methylated probes selected from those methylated probes listed in Table 4. The sequences of all the probes listed in Table 4 are also available from Illumina.

Prostate Cancer (PC) is a slow-growing malignancy, but acquires aggressive traits in a subset of cases. PCs have a propensity to be multifocal. Here, we show that DNA methylation of PC metastases is highly similar to specific primary foci, build an aggressiveness classifier, and determined PC aggressiveness of foci based on DNA methylation profiles in matched primary tumors and metastases. The classifier is significant associated with aggressiveness and the presence of lymph node metastases (P=9.2×10⁻⁵) and invasive tumor stages (P=7.7×10⁻⁷) in an independent cohort. Overall, this invention provides molecular-based support for determining PC aggressiveness with importance for clinical decision-making.

Other aspects and advantages of the invention will be apparent from the following description and the appended claims.

### BRIEF DESCRIPTION OF THE DRAWINGS

FIGS. 1A-1B. Strategy and sample selection. (FIG. 1A) Schematic representation of a prostate gland with four cancer foci (green and orange areas) and a pelvic lymph node with metastasis marked by a purple star. We discovered that we can determine the primary focus of metastasis origin based on DNA methylation in the lymph node metastasis, and this in turn will represent the most aggressive cancer subclone. By determining the aggressive subclone in multifocal PCs, we will obtain groups of aggressive and non-aggressive samples, which will form the basis for developing a classifier to determine the aggressiveness of primary PC foci. (FIG. 1B) An overview of the samples from patient 41 is shown in the upper left corner. P=patient, T=primary tumor focus, NL=tumor-negative lymph node, and PL=tumor-positive lymph node. The physical location of the five prostate samples and the two lymph node samples collected are shown on schematics of the dissected prostate gland (middle) and the lymphatic system (lower left corner), respectively.

FIGS. 2A-2B. DNA methylation patterns of lymph node metastasis indicate the potential primary focus/foci of origin. Left: Unsupervised clustering and heatmaps of all the samples from patient 41 (FIG. 2A) and patient 54 (FIG. 2B) based on the top 1% most variably methylated probes between all samples except the PLs. Dendrograms are shown above the heatmaps and the color key to the right. Right: Copy number alterations in Patient 41 (FIG. 2A) and Patient 54 (FIG. 2B). In each plot the samples were ordered based on the unsupervised clustering from the heatmaps to the left. The numbers and letters on the left of the plot designate the chromosome numbers. To the right is shown the color key; red=chromosomal gain and blue=chromosomal loss.

FIGS. 3A-3B. Building an aggressiveness classifier. (FIG. 3A) Phylogenetic reconstruction showing clonal relationships in each patient based on all filtered HM450 probes. Averaged normal prostate and normal lymph node samples were used for each tree. Sample types are colored with black (normal and PIN), orange (aggressive primary tumor), green (non-aggressive primary tumor), yellow shaded (undecided primary tumor), and purple (lymph node metastasis). Below each tree the longest Euclidean distance between any two samples in the tree are denoted so as to serve as a reference between the different trees. (FIG. 3B) MDS plot based on 25-probe classifier generated by GLMnet of the samples used for the analysis. The samples are separated into 3 distinct groups without overlap.

FIGS. 4A-4C. Validation of the PC aggressiveness classifier. (FIG. 4A) Manhattan plot of the probabilities calculated for 100 randomly selected samples from the TCGA PC cohort. Color bar in the bottom of the plot designates the sample types determined by TCGA. Black=Adjacent normal prostate. Yellow=Primary PC. Purple=PC Metastasis. The black dotted line marks the probability threshold used. (FIG. 4B) Distribution of the prediction of TCGA normal and tumor samples. (FIG. 4C) Evaluation of correctly predicted samples based on the histological microscopic examinations performed by TCGA.

FIGS. 5A-5E. Clinical information for predicted TCGA groups. (FIG. 5A) Pre-operative PSA among the aggressive (n=215) and non-aggressive (n=64) groups. Welch two sample t-test=0.005. Tumor size represented by the average intermediate dimension in cm among the aggressive (n=87) and non-aggressive (n=25) groups. Welch two sample t-test=0.9428. Percentage of patients with lymph node metastases at the time of surgery among the aggressive (n=187) and non-aggressive (n=52) groups. Fisher's exact 2-tailed P(Yes)=9.2×10-5. Pathological T stage distribution among the aggressive (n=217) and non-aggressive (n=64) groups. Fisher's exact 2-tailed P: P(T2)=2.2×10⁻⁷, P(T3)=7.7×10⁻⁷, P(T4)=0.6969. GS distribution among the aggressive (n=217) and non-aggressive (n=64) groups. Fisher's exact 2-tailed P: P(GS 6)=0.0591, P(GS 7)=0.2539, P(GS 8-10)=0.0220. P-values<0.05 are marked by an asterisk. (FIG. 5B) Distribution of Gleason 3+4 and 4+3 tumors among the aggressive (n=96) and non-aggressive (n=34) groups. Fisher's exact 2-tailed P(3+4)=0.8424. P-values<0.05 are marked by an asterisk. Pathological T stage of Gleason 7 tumors among the aggressive (n=96) and non-aggressive (n=34) groups. Fisher's exact 2-tailed P: P(T2)=1.5×10-4, P(T3)=1.2×10-4, P(T4)=1. In FIG. 5C, FIG. 5D and FIG. 5E, Fisher's exact 2-tailed tests were used to calculate significance. P-values<0.05 were considered significant.

FIGS. 6A-6B. (FIG. 6A) Boxplot of 500 probes methylated in prostate tissue and unmethylated in peripheral blood. The normal lymph node samples had median DNA methylation beta values of −0.2. Any of the other samples with medians below 0.6 were excluded from further analysis: P15_PL and P32_PL.Tumor purity. (FIG. 6B) Boxplot of GSTP1 promoter DNA methylation for all samples. Tumor samples with median DNA methylation below 0.4 (dotted vertical line) were excluded from analysis: P17_T3 and P23_T3. The two PIN lesions were both just below 0.4 and were kept for further analysis.

FIG. 7. Sample dissimilarity. MDS plot drawn from Euclidean distances calculated based on all filtered probes.

FIG. 8. Unsupervised clustering and heatmaps. Clustering and heatmap visualization for all samples from Patients 14, 17, 23, 24, 26, 43, 52, 56, 84, 85, 88, and 98 was based on the top 1% most variably methylated probes between all samples, except the PLs. Dendrograms are shown above the heatmaps and the color key to the right.

FIG. 9. Input probes for GLMnet analysis. Heatmap of the top 3,000 most differentially methylated probes between the aggressive (n=31) and non-aggressive (n=10) groups based on the average methylation differences. Sidebar on the top shows the group of each sample (same as in FIGS. 3A-3B).

FIGS. 10A-10B. Clinical characteristics of TCGA PC GS 7 tumors. (FIG. 10A) Pathological T stage of Gleason 7 tumors among the aggressive (n=96) and non-aggressive (n=34) groups. Fisher's exact 2-tailed P: P(T2)=1.5×10⁻⁴, P(T3)=1.2×10⁻⁴, P(T4)=1. (FIG. 10B) Distribution of Gleason 3+4 and 4+3 tumors among the aggressive (n=96) and non-aggressive (n=34) groups. Fisher's exact 2-tailed P(3+4)=0.8424. P-values<0.05 are marked by an asterisk.

FIGS. 11A-11C. DNA methylation of metastasis and primary site from the same patient is highly similar. (FIG. 11A) Between-sample correlation plot. Sample names are shown to the left of the plot. At the top and the left of the plot are colored sidebars showing sample type and patient identifier. The sidebar to the right of the plot shows the correlation coefficient color key; red being high correlation and blue low correlation. P=patient, AN=adjacent normal, T=primary tumor focus, NL=tumor-negative lymph node, and PL=tumor-positive lymph node. (FIG. 11B) Enlargement of correlation amongst primary tumor foci in patient 41. (FIG. 11C) Enlargement of correlation between all primary tumor foci and all positive lymph nodes.

FIG. 12. Input probes for GLMnet analysis. Heatmap of the top 3,000 most differentially methylated probes between the aggressive (n=31) and non-aggressive (n=10) groups based on the average methylation differences. Sidebar on the top shows the group of each sample.

### DETAILED DESCRIPTION OF THE INVENTION

A “biomarker” as used herein refers to a molecular indicator that is associated with a particular pathological or physiological state. The “biomarker” as used herein is a molecular indicator for cancer, more specifically an indicator for prostate cancer.

As used herein the term “cancer” refers to or describes the physiological condition in mammals that is typically characterized by abnormal and uncontrolled cell division or cell growth. Examples of cancer include but are not limited to, carcinoma, lymphoma, blastoma, sarcoma, and leukemia.

As used herein, a “subject” is preferably a human, non-human primate, cow, horse, pig, sheep, goat, dog, cat, or rodent. In all embodiments, human subjects are preferred. The “subject” may be at risk of developing prostate cancer, may be suspected of having prostate cancer, or may have prostate cancer. In addition, a “subject” may simply be a person who wants to be screened for prostate cancer.

### Example 1

### DNA Methylation Patterns of Lymph Node Metastases Indicate the Potential Primary Focus/Foci of Origin

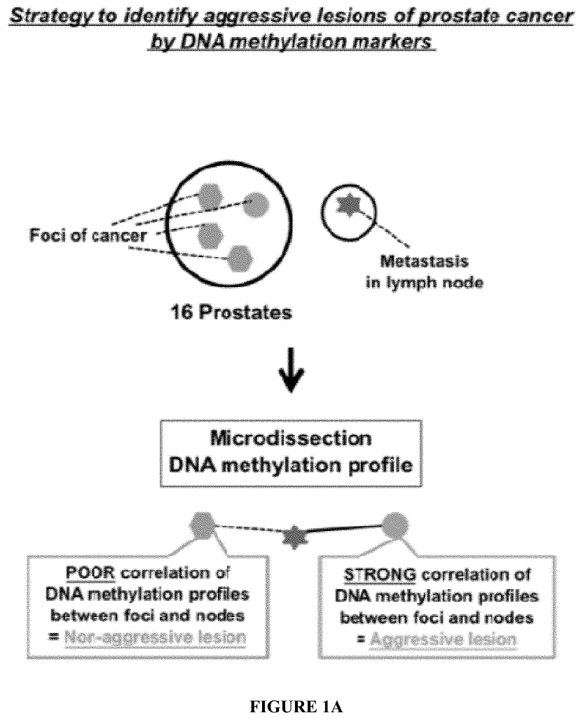

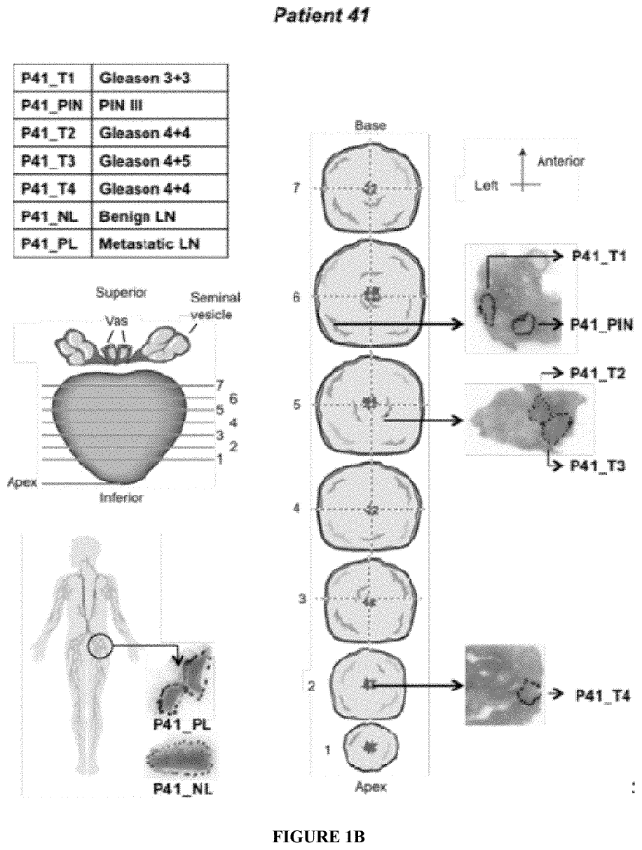

In this study, aggressive primary cancer focus/foci can be identified from multifocal PC by the degree of correlation of DNA methylation to lymph node metastases, which represent an aggressive trait (FIG. 1A). The study relies on four assumptions: 1) A subset of multifocal PCs arise from independent and sporadic genetic/epigenetic changes, effectively implying that distinct cancer foci develop through different molecular mechanisms/pathways and harbor unique proliferation, migration, and aggressiveness potential; 2) DNA methylation changes hold information about clonal evolution and will not change substantially upon dissemination^{11,28,29}; 3) metastases have the same clonal origin^{27,28}; and 4) the pelvic lymph nodes drain from a cancerous prostate and are likely the first location for development of metastases. Thus, lymph node metastases are herein defined as an aggressive trait and are used as an indicator of the ability to establish distant metastases as well.

We used the Illumina HumanMethylation450 BeadArray (HM450) platform to measure the DNA methylation status of matched primary tumors and pelvic lymph node metastases in 16 patients with multifocal disease (Table 1). The prostate and lymph node tissue samples stored in FFPE tissue blocks were sectioned, H&E stained (FIG. 1B), and examined by a trained pathologist. All areas of cancer were marked and given a GS. In addition, areas of adjacent normal prostate cells and prostatic intraepithelial neoplasia (PIN) were marked when possible, summing to a total of 92 samples (Methods).

TABLE 1
Patient Information

| | | Radical | | | | | |
|---|---|---|---|---|---|---|---|
| Patient | Age | Prostatectomy | Tumor | Gleason | Nodal | Malignant | Patient Status |
| no. | (years) | (year) | Stage | Score | Stage | LNs | (2015) |
| 14 | 64 | 2013 | pT3a | 4 + 5 | pN1 | 2/16 | Alive |
| 15 | 63 | 2007 | pT3a | 3 + 4 | pN1 | 2/9 | Alive |
| 17 | 61 | 2000 | pT3b | 3 + 5 | pN1 | 2/22 | Dead |
| 23 | 68 | 2004 | pT3 | 4 + 5 | pN1 | 14/49 | Dead |
| 24 | 66 | 2013 | pT3 | 3 + 4 | pN1 | 1/9 | Alive |
| 26 | 60 | 2013 | pT3b | 4 + 5 | pN1 | 3/45 | Alive |
| 32 | 66 | 2013 | pT3b | 4 + 4 | pN2 | 8/32 | Alive |
| 41 | 68 | 1999 | pT3b | 3 + 5 | pN1 | 6/11 | Dead |
| 43 | 62 | 2004 | pT3b | 5 + 4 | pN1 | 25/51 | Unknown |
| 52 | 75 | 2013 | pT3b | 5 + 4 | pN1 | 14/42 | Alive |
| 54 | 55 | 2008 | pT4 | 5 + 5 | pN1 | 4/8 | Dead |
| 56 | 57 | 2013 | pT3b | 5 + 4 | pN1 | 11/43 | Alive |
| 84 | 58 | 2008 | pT3b | 5 + 4 | pN1 | 4/50 | Alive |
| 85 | 61 | 2013 | pT3b | 4 + 4 | pN1 | 4/29 | Alive |
| 88 | 65 | 1991 | pT3b | 4 + 5 | pN2 | 5/20 | Dead |
| 98 | 79 | 2013 | pT3b | 4 + 5 | pN1 | 8/23 | Alive |

LN = lymph node

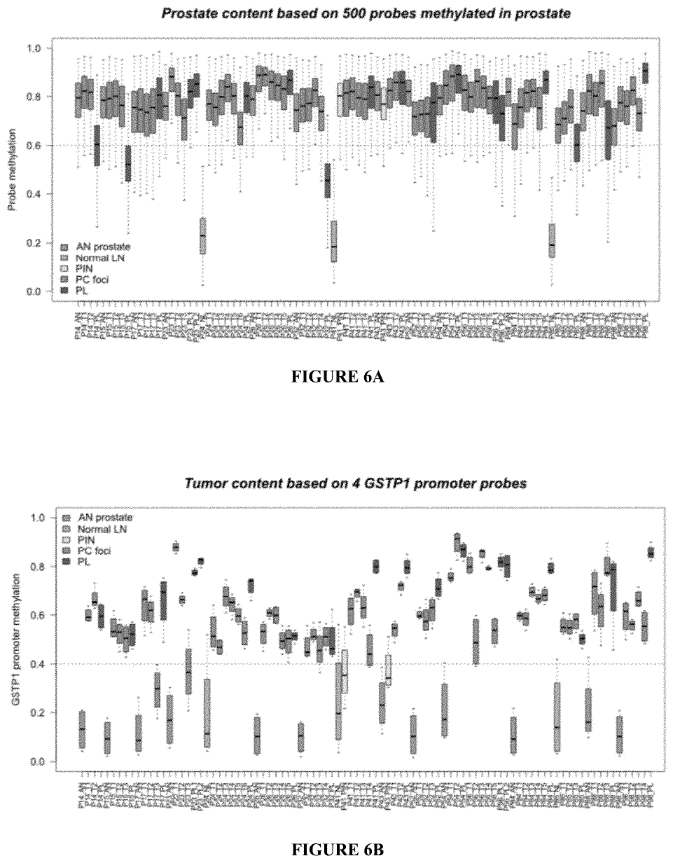

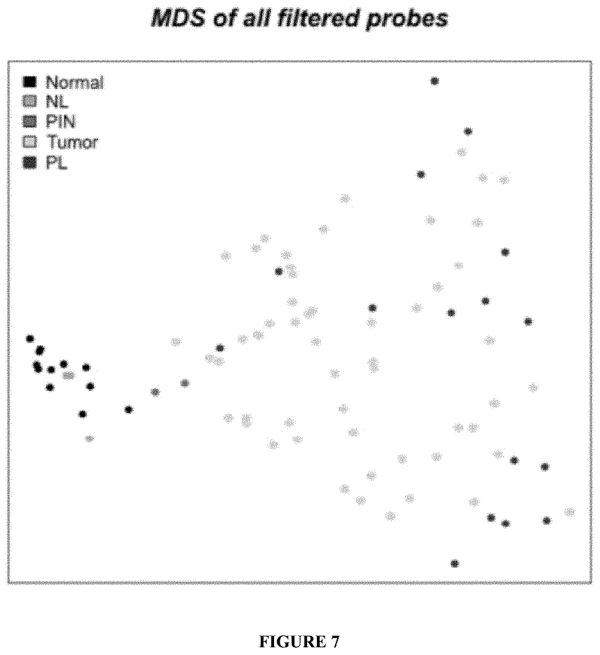

Sample purity was tested with respect to either infiltration of normal cells or leukocytes caused by inflammation (Methods, FIG. 6A-6B). Two primary tumor foci were removed due to low tumor cell content (P17_T3 and P23_T3), and two positive lymph node (PL) metastases were removed due to high leukocyte content (P15_PL and P32_PL), thereby excluding all samples from Patients 15 and 32. HM450 DNA methylation data from the remaining 14 patients were compared in a multidimensional scaling (MDS) plot, in which samples are placed in two-dimensional space based on dissimilarity (FIG. 7). Primary tumors and lymph node metastases were highly heterogeneous with no obvious subgroups, whereas normal prostate and lymph node tissues formed a tight cluster, as expected, indicating that the experimental protocol was successful.

In order to investigate if DNA methylation patterns hold information about clonal evolution in PC, Pearson correlations amongst all the samples were calculated, plotted, and visualized using heatmaps (FIG. 11A). Firstly, primary foci from the same patient showed more variable correlation coefficients (0.89-0.99), as compared to for interpatient AN-AN samples (0.96-0.99) and interpatient AN-NL samples (0.90-0.94), indicating that multiple cancer subclones are present in some patients (FIG. 11B) and in turn may hold distinct tumorigenic potential. Secondly, lymph node metastases consistently showed the highest correlation to one or more of the primary tumor foci from the same patient (0.94-0.98, FIG. 11C). Thus, DNA methylation profiles had not diverged to such a degree that metastases and primary tumors remained comparable. Taken together, these results demonstrate that a subset of multifocal PCs show independent epigenetic changes, indicating that cancer foci develop from unique subclones. Furthermore, the DNA methylation profiles of lymph node metastases are highly correlative to a focus/foci from individual patients.

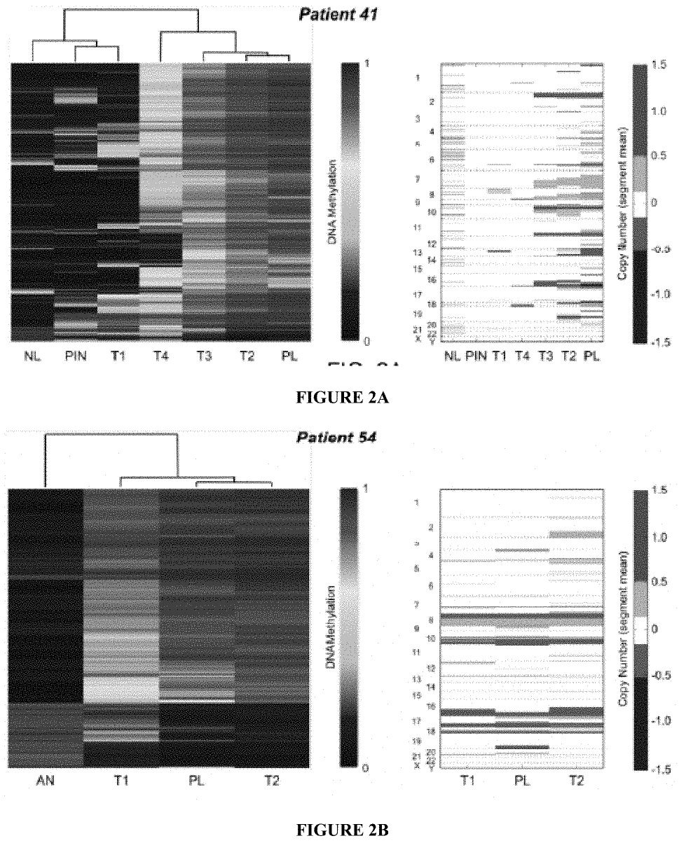

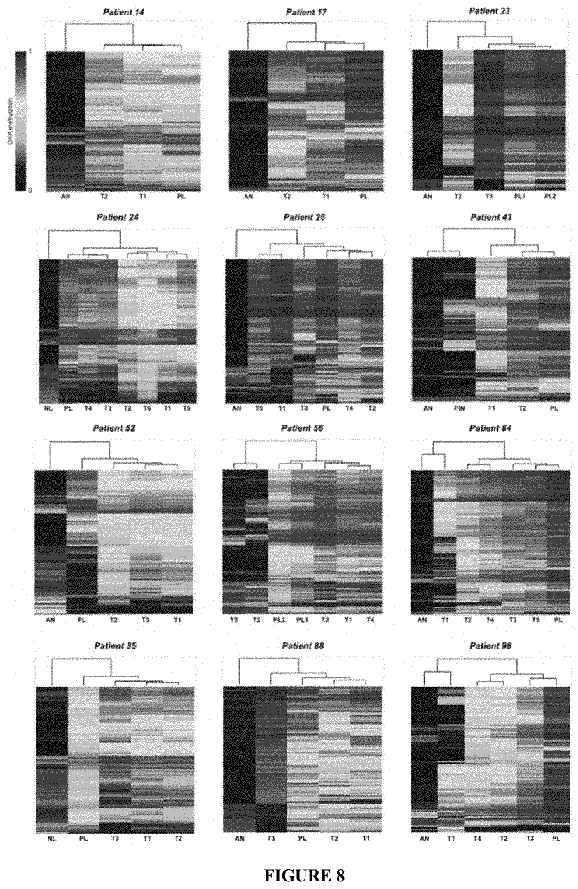

We first investigated the DNA methylation profiles of PC foci among individual patients. To identify the focus of origin of lymph node metastasis, we selected the top 1% most variably methylated probes between all samples for each patient, excluding PLs. The DNA methylation levels of these probes from all samples, including PLs, were then compared by unsupervised hierarchical clustering and heatmap visualization. Based on similar DNA methylation levels, PLs clustered with other samples, thereby providing information about the potential clonal relationship. Heatmaps after unsupervised clustering of these probes for two representative patients, Patients 41 and 54 (FIG. 2A and FIG. 2B, left panels), as well as for the remaining 12 patients with lymph node metastases (FIG. 8), showed PLs clustering with one or more of the primary tumor foci, whereas no PLs clustered with the adjacent normal prostate tissues, normal lymph nodes, or PIN lesions. In all 14 cases with lymph node metastases, the PLs clustered with one or more of the primary tumor foci, and no PLs clustered with the adjacent normal prostate tissues, normal lymph nodes, or PIN lesions (FIG. 2A, FIG. 2B and FIG. 8). In addition, PLs were found to cluster and were highly correlated in two patients (P23 and P56) with multiple PLs (0.99 and 0.98, respectively; FIG. 8), supporting the assumption (#3) that metastases have the same clonal origin.

The PL DNA methylation profile for Patient 41 was very similar to and clustered very closely with the T2 and T3 foci, while the T4 and T1 foci were more dissimilar, as shown by the dendrogram at the top of the heatmap. For this patient, the T2 and/or T3 foci are the most likely origin(s) of the metastasis. Furthermore, the physical juxtaposition of T2 and T3 in the prostate specimen (FIG. 1B) may suggest that they diverged from the same population of transformed cells during tumorigenesis. For Patient 54, which only had two primary foci, the PL DNA methylation was very similar to both the T1 and T2 foci. Hence, both patients displayed multiple primary tumor foci with very similar DNA methylation profiles, indicating a monoclonal origin of these foci. In addition, Patient 41 also displayed tumor foci with very different DNA methylation profiles, indicating the occurrence of multiple independent transformation events and therefore, multiple subclones.

In order to validate these findings, we took advantage of the recent evidence that the HM450 DNA methylation platform can also be used to determine CNAs by summing the methylated and unmethylated signal intensities of the probes^{30,31}. This analysis provided additional evidence that the T2 and T3 foci were very similar to the PL in Patient 41. All had deletions on chromosomes 2, 10, 11, and 16 and gains on chromosomes 7, 8, and 10, however, these regions were not altered in the T1 or T4 foci, which show different CNA patterns (FIG. 2A, right panel). All three samples from Patient 54 show multiple shared alterations, as well as deletion of the short arm and amplification of the long arm of chromosome 8, both common features of PC^{32,33} (FIG. 2B, right panel). Thus, the CNA analysis supports our findings of multiple subclonal origins in Patient 41 (FIG. 2A) and a monoclonal origin in Patient 54 (FIG. 2B) using DNA methylation analysis. Moreover, the CNA results also support our finding that the origin of lymph node metastasis can be determined by DNA methylation.

Similarly, all PLs clustered with one or more primary tumor foci from the remaining 12 cases using our DNA methylation-based approach (FIG. 8). Furthermore, nine patients (P23, P24, P26, P41, P43, P56, P84, P88, and P98) showed clearly distinct DNA methylation patterns among the primary foci, indicating the existence of independent tumor subclones. Taken together, these results suggest that the DNA methylation pattern of a lymph node metastasis can be used to identify the potential primary focus/foci of origin of metastasis, and that PC patients may contain subclones with aggressive and non-aggressive potential.

### Example 2

### Development of a Panel of DNA Methylation Markers as a Classifier for Pc Aggressiveness

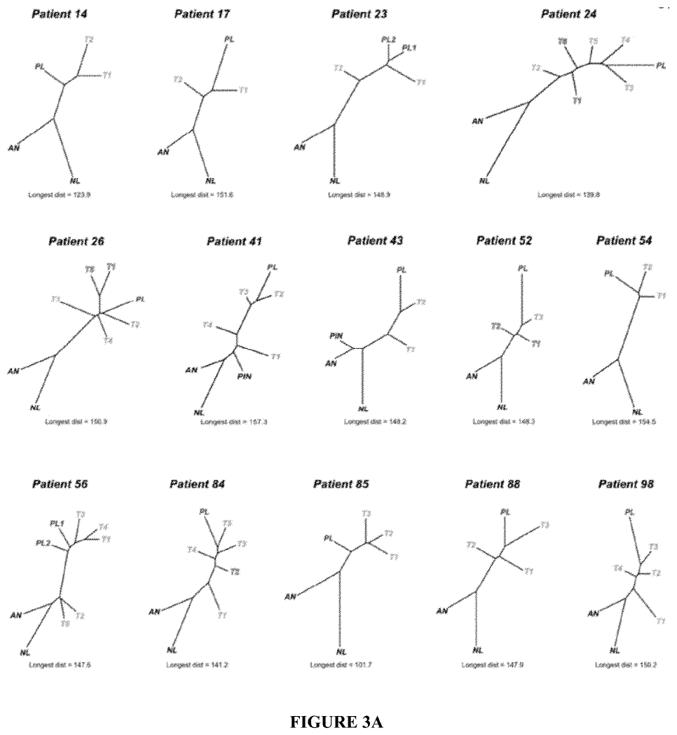

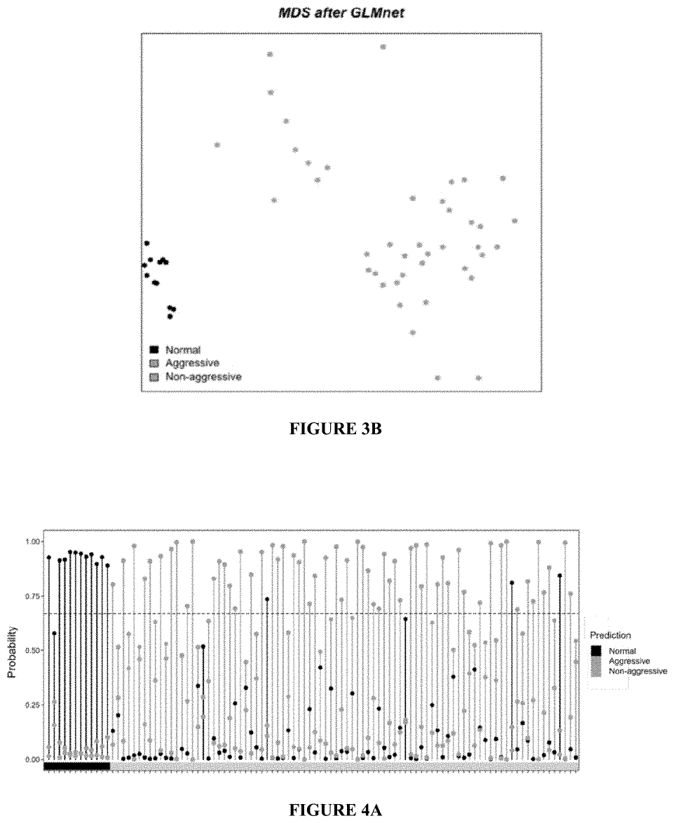

We next devised a DNA methylation-based PC aggressiveness classifier to categorize primary tumor foci as either aggressive or non-aggressive. The unsupervised hierarchical clustering approach effectively identifies the primary origin of lymph node metastases. However, in order to categorize the aggressiveness of individual foci in a quantitative, unbiased, and objective manner, we calculated Euclidean distances between any two samples within a patient using all filtered HM450 probes. Euclidean distance, like Pearson correlation, compares sample similarities, but maintains data variability, and is also superior for analysis of differential expression³⁴. We divided the scale of Euclidean distances into discrete categories (aggressive, undecided, and non-aggressive) for all primary tumor foci. Since the purpose of this categorization method is to assemble groups of genuinely aggressive and non-aggressive tumors for biomarker development, we included a gap of 10 Euclidean distance units (undecided category) to reduce the risk of misclassification. Sample categorization for each patient is shown using DNA methylation-based phylogenetic trees where samples are colored as a function of aggressiveness (FIG. 3A and overview in Table 2).

TABLE 2
PC Foci Aggressiveness by Patient

| | | No. of | No. of Non- |
|---|---|---|---|
| Patient | Multiple | Aggressive | Aggressive |
| No. | Subclones | Subclones | Subclones |
| 14 | No | 2 | 0 |
| 17 | No | 2 | 0 |
| 23 | Yes | 1 | 1 |
| 24 | Yes | 3 | 1 |
| 26 | Yes | 2 | 1 |
| 41 | Yes | 2 | 2 |
| 43 | Yes | 1 | 1 |
| 52 | ? | 1 | 0 |
| 54 | No | 2 | 0 |
| 56 | Yes | 3 | 2 |
| 84 | Yes | 3 | 1 |
| 85 | No | 3 | 0 |
| 88 | No | 3 | 0 |
| 98 | Yes | 3 | 1 |

Taken together, our developed categorization approach found that eight patients (Patients 23, 24, 26, 41, 43, 56, 84, and 98) showed independent DNA methylation profiles indicative of multiple subclones. Five patients (Patients 14, 17, 54, 85, and 88) showed similar DNA methylation patterns indicating a monoclonal origin, and one patient (Patient 52) was not categorized as either (FIG. 3A and Table 2). These findings are in agreement with the unsupervised clustering data (FIG. 2A, FIG. 2B and FIG. 8) with the exception of Patient 88, which did not show discrete subclones as indicated by the heatmap and dendrogram. In this patient, the top 1% most variably methylated probes were not representative of the potential clonal relationship.

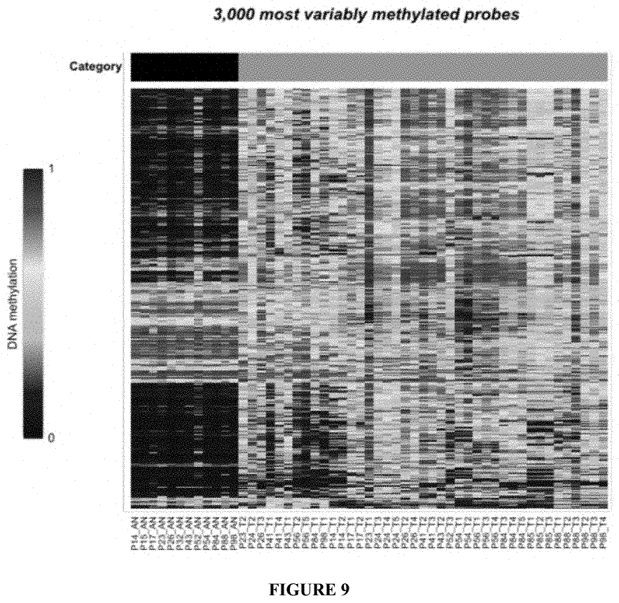

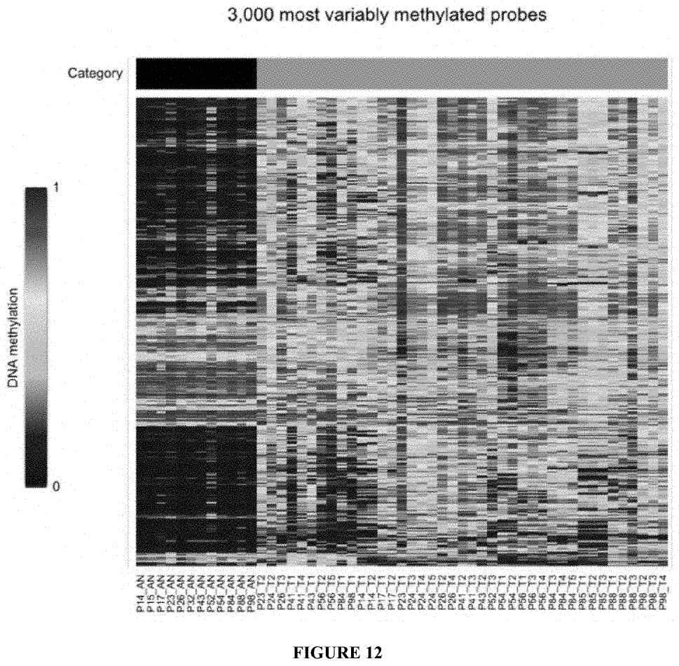

We next searched for differentially methylated probes between the aggressive and non-aggressive groups (FDR adjusted p<0.05), but found that the DNA methylation levels of no single probe were significantly different between the two groups. Using an FDR cutoff of 0.3, 231 probes were identified. Still, we continued to search for a set or panel of probes able to distinguish these groups from a larger panel. First, we generated a list of the 3,000 most differentially methylated probes between the assembled aggressive and non-aggressive groups based on mean DNA methylation differences (FIG. 9, FIG. 12, Table 4), which was subsequently used as input for the GLMnet algorithm35 along with information about normal, aggressive, and non-aggressive sample groups. The GLMnet model generates outputs in the form of probabilities of group membership, which are functions of the DNA methylation values for a given set of probes that differentiate the groups. For each sample, the probabilities of normal, aggressive, and non-aggressive groups sum to 1, and the group with the highest probability is the predicted phenotype of a given sample. Upon numerous iterations and refinement of the input probes list (Materials and Methods), we found a set of 25 probes (Table 3) that optimally predict normal, non-aggressive, and aggressive categories (FIG. 3B). Twenty-one (84%) of the 25 probes in the classifier were among the probes with FDR-adjusted p<0.3 for either aggressive versus non-aggressive, aggressive versus normal, or non-aggressive versus normal comparisons.

TABLE 3
25-Probe Aggressiveness Classifier

| | | | UCSC | UCSC | RELATION |
|---|---|---|---|---|---|
| | | | REFGENE | REFGENE | TO UCSC |
| TargetID | Specificity | Methylation | NAME | GROUP | CPG ISLAND |
| cg00697992 | Normal | Hypo | NXPH2 | Body | N_Shore |
| cg01272707 | Aggressive | Hyper | — | — | Island |
| cg01819167 | Aggressive | Hypo | — | — | — |
| cg01906055 | Aggressive | Hypo | NCAPH | TSS1500 | N_Shore |
| cg02160684 | Aggressive | Hypo | TRIB1 | Body | S_Shelf |
| cg02560085 | Non-aggressive | Hyper | PCDHA1- | TSS200, | N_Shore |
| | | | PCDHA8 | Body | |
| cg03195164 | Non-aggressive | Hyper | C3orf37 | Body | S_Shore |
| cg03456213 | Non-aggressive | Hyper | C9orf3 | Body | Island |
| cg04634417 | Aggressive | Hypo | PCDHA1- | TSS200, | N_Shore |
| | | | PCDHA8 | Body | |
| cg06024295 | Aggressive | Hyper | CPN1 | 1st Exon, | — |
| | | | | 5′ UTR | |
| cg11748187 | Non-aggressive | Hyper | TCF7L2 | Body | S_Shore |
| cg13300630 | Aggressive | Hyper | ROBO1 | Body | — |
| cg13944838 | Non-aggressive | Hypo | GFPT2 | Body | Island |
| cg14399930 | Aggressive | Hyper | FBXO47 | TSS200 | — |
| cg15132013 | Aggressive | Hypo | SKI | Body | S_Shelf |
| cg16469740 | Aggressive | Hyper | HDAC9 | Body | — |
| cg17004353 | Aggressive | Hypo | CARS | 5′ UTR, | — |
| | | | | Body | |
| cg17032646 | Aggressive | Hyper | SLC6A17 | TSS1500 | N_Shore |
| cg18315943 | Aggressive | Hypo | — | — | Island |
| cg19550524 | Aggressive | Hypo | — | — | — |
| cg20399616 | Normal | Hypo | BCAT1 | Body | Island |
| cg24517686 | Aggressive | Hypo | — | — | S_Shore |
| cg25961816 | Non-aggressive | Hypo | — | — | — |
| cg26447413 | Non-aggressive | Hyper | GAS1 | 1st Exon | Island |
| cg27198013 | Non-aggressive | Hypo | RAI1 | 3′ UTR | Island |

One of ordinary skill in the art will recognize that the methylated probe can also include sequence regions that extend upstream and/or downstream from the methylated probe. For example, the probe region could also include 250 bases (or greater) upstream and/or downstream from the probe, 200 bases upstream and/or downstream from the probe, 100 bases upstream and/or downstream from the probe, etc.

### Example 3

### The Tcga Pc Cohort Validates the Potential of the Aggressiveness Classifier

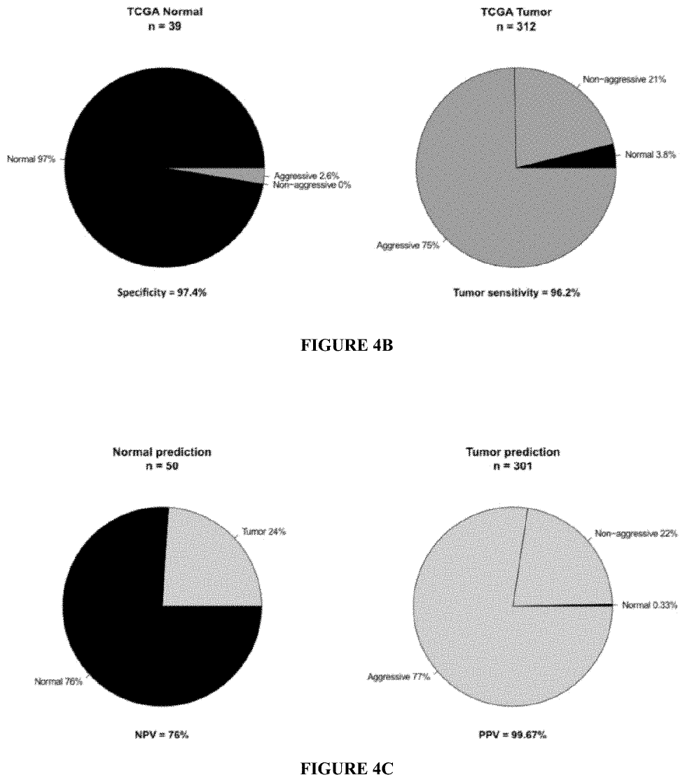

To test the classifier on an independent dataset, we took advantage of the publically available prostate adenocarcinoma HM450 DNA methylation data and accompanying clinical information from The Cancer Genome Atlas (TCGA) project. We tested 496 prostate samples (adjacent normal and tumor) using the classifier, and 70% of the samples (351 samples; 39 adjacent normal and 312 tumor samples) were predicted with a probability above 0.67 (FIG. 4A). Of the 39 normal TCGA samples, 38 were predicted as normal, and one as aggressive. Of the 312 tumors, 233 were predicted as aggressive, 67 were predicted as non-aggressive, and 12 were predicted as normal, resulting in a 97.4% specificity and a 96.2% sensitivity for PC as compared to adjacent normal tissue samples (FIG. 4B). Upon evaluation of the consistency between our predictions and the sample diagnoses (PC vs. AN) based on the histological microscopic examinations performed by TCGA, the classifier has a 76% negative predictive value (NPV) and a 99.7% positive predictive value (PPV; FIG. 4C).

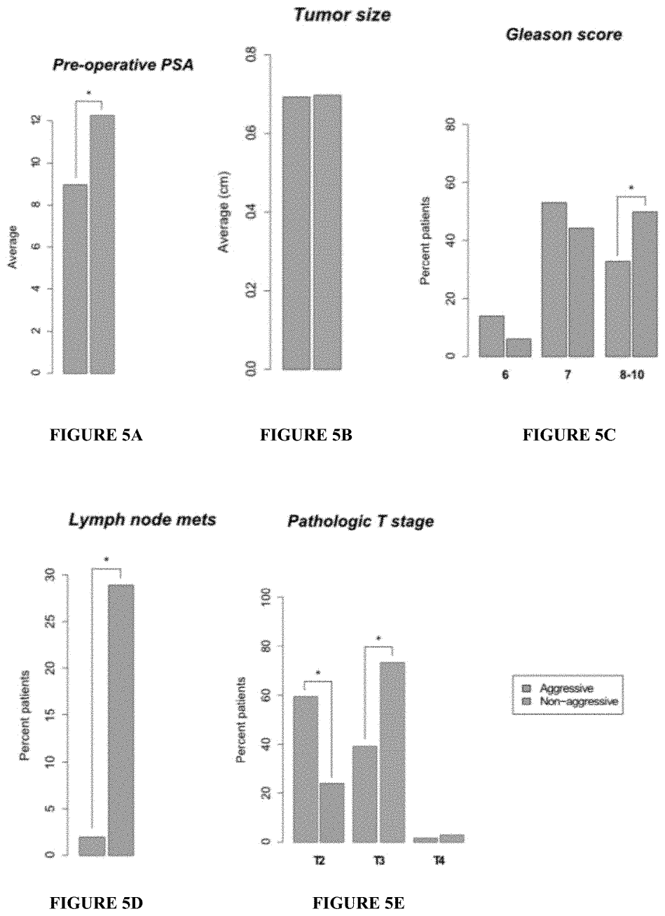

To evaluate the prognostic performance of the classifier, we consulted available clinicopathological co-variates known to be associated with PC aggressiveness (pre-operative PSA, tumor size, pathological GS, presence of lymph node metastases, and tumor stage) for samples with probabilities above 0.67. Aggressiveness was significantly (P<0.02) associated with the investigated co-variates except tumor size (FIG. 5A-5E). Pre-operative PSA levels were higher in the aggressive group compared to the non-aggressive group (P=0.005; FIG. 5A). However, similar tumor sizes between groups (FIG. 5B) indicates that aggressiveness and tumor size are independent as has also been suggested previouslyl³.

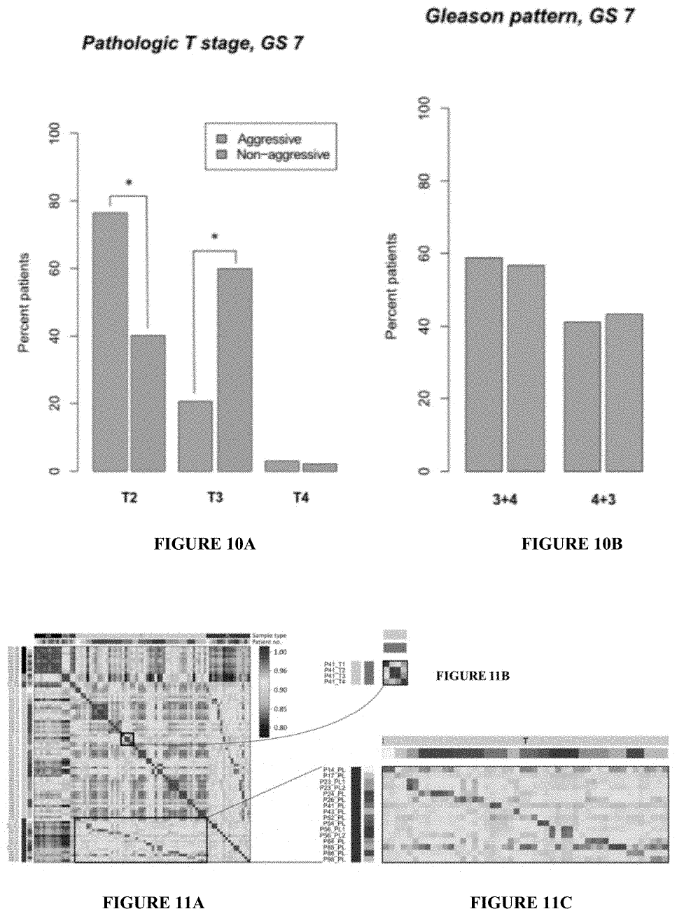

Upon further examination, tumors with high GSs (GS 8-10) were significantly associated with the aggressive group (P=0.022), but no such association was seen for tumors with low (GS 6) and intermediate (GS 7) scores (P=0.059 and P=0.254, respectively; FIG. 5A). GSs are well correlated with PC aggressiveness, especially at the low (GS 6) and high (GS 8-10) ends of the scale^{5,6}, and Gleason scoring is a valuable tool in PC treatment. However, additional information is required to determine aggressiveness for the intermediate (GS 7) tumors. Interestingly, the GS 7 tumors, which comprise nearly one-half of all TCGA PC tumors, were not significantly associated with non-aggressive or aggressive groups (FIG. 5A), indicating that this large group in particular may benefit from our DNA methylation-based classifier in order to determine whether active surveillance or ablative treatment is the best course of action. In support of this, we also found that the GS 7 tumors classified as non-aggressive were significantly associated with tumor stage T2 (P=1.5×10-4), while GS 7 tumors classified as aggressive were significantly associated with tumor stage T3 (P=1.2×10-4; FIG. 5B, FIG. 10A). Furthermore, we tested whether the primary and secondary patterns of the GS 7 tumors showed a correlation to the aggressive or non-aggressive groups (FIG. 5B, FIG. 10B). GS is calculated by summing the primary (largest pattern) and secondary (second largest pattern) Gleason grades, each of which ranges from 1 (well differentiated) to 5 (poorly differentiated)⁵. Interestingly, there was no difference in the distribution between 3+4 and 4+3 tumors and indicates that tumors of this large intermediate Gleason 7 group can be further and more accurately stratified using our molecular-based classifier to help determine whether active surveillance or ablative treatment should be performed.

Importantly, we found that significantly more patients classified as having an aggressive PC presented with lymph node metastases at the time of surgery as compared to patients with predicted non-aggressive tumors (P=9.2×10⁻⁵; FIG. 5D). Also, the pathological evaluation of tumor stage (FIG. 5E) showed significantly more organ-confined stage T2 tumors in the non-aggressive group (P=2.2×10⁻⁷) and significantly more of the capsule-penetrating and seminal vesicle invasive stage T3 tumors in the aggressive group (P=7.7×10⁻⁷). This strong correlation holds great promise for our classifier if developed into a molecular DNA methylation-based assay for needle biopsy samples, since the pathological tumor stage cannot be obtained until after surgery. Thus, if classification data had been obtained from needle biopsy samples prior to surgery, our recommendation would place ˜25% (67+12/312) of patients, the majority with GSs 6 and 7, in an active surveillance regimen rather than proceed with prostate ablation.

### Example 4

### Methods

### Patient Material, Samples, and Marking

Sixteen (16) patients diagnosed with multifocal PC having metastasized to one or more pelvic lymph nodes were enrolled in the study (Table 1). All patients had radical prostatectomies and removal of pelvic lymph nodes in the period between 1991-2013. No antiandrogen treatments were administered prior to surgery. The prostate and lymph node tissue samples were stored in FFPE tissue blocks. FFPE blocks were sectioned and H&E stained (FIG. 1B). A trained pathologist examined all slides covering the entire prostate and dissected lymph nodes, and all areas of cancer were marked and given a GS. In addition, areas of normal and PIN were marked when possible, adding up to a total of 92 samples. The marked H&E slides were used to guide the dissection of AN/PIN/PC cells from 8-10 unstained slides (5-10 μm).

### Deparafinization and Purification

The dissected tissue samples were deparaffinized using a double xylene wash followed by a double ethanol wash and drying of the pellets. For DNA extraction, the pellets were resuspended in 240 μl of PKD buffer and Proteinase K (Qiagen, miRNeasy FFPE kit (cat no. 217504)), and then incubated at 55° C. overnight and 85° C. for 15 min. After cooling the samples, 500 μl RBC buffer was added and the samples were run through gDNA Eliminator columns (RNeasy plus mini kit (cat no. 74134)) using RPE buffer to wash and EB buffer for elution.

### DNA Methylation Profiling

Genomic DNA (200-500 ng) from each FFPE sample was treated with sodium bisulfite and recovered using the Zymo EZ DNA methylation kit (Zymo Research, Irvine, Calif.) according to the manufacturer's specifications and eluted in 10 μl volume. An aliquot (1 μl) was removed for MethyLight-based quality control testing of bisulfite conversion completeness and the amount of bisulfite converted DNA available for the Infinium Methylation assay⁴². All samples that pass the QC tests were then repaired using the Illumina Restoration solution as described by the manufacturer. Each sample was then processed in the Infinium DNA methylation assay data production pipeline⁴³.

After the chemistry steps, BeadArrays were scanned and the raw signal intensities were extracted from the *.IDAT files using the R package methylumi. The intensities were corrected for background fluorescence and red-green dye-bias⁴⁴. The beta values were calculated as (M/(M+U)), in which M and U refer to the (pre-processed) mean methylated and unmethylated probe signal intensities, respectively. Measurements in which the fluorescent intensity was not statistically significantly above background signal (detection P-value>0.05) were removed from the data set. In addition, probes that overlap with known SNPs as well as repetitive elements were masked prior to data analyses. Specifically, all HM450 probes that overlapped with common SNPs with a minor allele frequency of greater than 1% (UCSC criteria) at the targeted CpG site, as well as probes with SNPs (MAF>1%) within 10 bp of the targeted CpG site were masked. HM450 probes that were within 15 bases of the CpG lying entirely within a repeat region were also masked prior to data analyses. The end result was a dataset of corrected beta-values for 396,020 probes which cover 21,000 genes.

### Calculation of Tumor Purity

To investigate the degree of leukocyte infiltration in each sample, public HM450 data from 96 male peripheral blood samples (GSE53740 and GSE51388) were downloaded using Marmal-aid⁴⁵. All HM450 probes with beta values>0.2 in male peripheral blood were excluded. The remaining probes were used to subset 500 probes that were hypermethylated in 43 normal prostate samples from TCGA, and thus hypomethylated in blood. Tissues of prostate origin from our study with mean DNA methylation of these probes below 0.6 were excluded from further analysis. Two lymph node metastases were excluded due to high blood content. Four GSTP1 HM450 probes (cg06928838, cg09038676, cg22224704, cg26250609) were used for tumor purity analysis as described in Brocks et al.¹¹. Primary tumors with mean DNA methylation below 0.4 were excluded from further analysis. Two tumor samples were excluded due to high normal content.

### Unsupervised Hierarchical Clustering

For each patient, probes with missing beta values (detection P-value>0.05) were excluded and the top 1% most variably methylated probes between all the samples except the PL(s) were selected. Heatmaps were used to display the DNA methylation levels and the unsupervised hierarchical clustering was performed with the hclust function in R (method=“complete”).

### Copy-Number Alteration Analysis

The CNAs were analyzed using the Champ package for R⁴⁶ using 28 adjacent normal prostate samples purified from FFPE tissues (12 from this study and 16 from own unpublished data) as a reference. Imported beta values were run through champ.norm and champ.CNA (filterXY=FALSE, batchCorrect=T, freqThreshold=0.3). The generated segment mean-files were intersected with the Infinium probe locations using BedTools and the resulting chromosomal loss and gain was illustrated in heatmaps using Matlab. Most of the samples showed noisy profiles, likely due to DNA breakage accumulated during the storage in FFPE, and the analysis could not be completed for all samples.

### PC Tumor Aggressiveness Categorization

Euclidean distances were calculated between any two samples using all 396,020 filtered probes. Averaged normal prostate and normal lymph node samples showed minimal variance and were used for the analysis. Normal prostate was considered to be very similar because only 0.65% (2561/396020) of standard deviations of all the probes were above 0.15. Normal lymph nodes were considered to be very similar because only 0.98% (3875/396020) of standard deviations of all the probes were above 0.15. The primary focus with the shortest Euclidean distance to the lymph node metastasis (T-PL dist 1) was categorized as aggressive. The additional distance to the other primary foci (T-PL dist 2; actual T-PL dist-T-PL dist 1=T-PL dist 2) were assessed in a density graph and a division of the scale based hereon. If T-PL dist 2s were only 0-10 units longer, they were also categorized as aggressive. This ensured that the foci of monoclonal origin would all be grouped as aggressive. Next, T-PL dist 2s longer by >20 units were categorized as non-aggressive origins and T-PL dist 2s of between 10-20 were categorized as undecided (overview in Table 2). In the two patients with 2 PLs the division of the primary tumors was done based on the PL with the shortest distance to a primary focus, which was P23_PL2 and P56_PL1.

### Phylogenetic Reconstruction

Methylation-based phylogenetic trees were inferred by the minimal evolution method⁴⁷. Euclidean distances were calculated using all 396,020 filtered probes.

### Calculation of Differential Methylation

Differential methylation between any two groups of samples were calculated using the champ.MVP( ) function from the ChAMP package utilizing either FDR<0.05 or FDR<0.3.

### Developing the DNA Methylation-Based PC Aggressiveness Classifier

By combining the categorized samples into groups of aggressive (n=31) and non-aggressive (n=10), we generated a list of 3,000 most variably methylated CpG sites (probes) between the groups. This list was used as input for the GLMnet algorithm³⁵, along with normal (n=12), non-aggressive (n=10), and aggressive (n=31) prostate sample groups. The GLMnet algorithm outputs a set of probes able to differentiate groups of samples based on their DNA methylation. Following 15 iterations, each output was evaluated by 1) the separation of the three groups (input as normal, aggressive, non-aggressive) in MDS plots like in FIG. 3B and FIG. 9; 2) the DNA methylation levels of the probes in heatmaps; 3) the prediction probabilities in Manhattan plots like in FIG. 4A for each set of probes run back on the input samples. This resulted in a refinement of the list of input probes from 3,000 to 39. Rerunning the GLMnet algorithm utilizing the 39 probes as input, a set of 25 probes (Table 3) was found to be the optimal predictor of our sample set according to normal, non-aggressive, and aggressive categories.

### Testing the Aggressiveness Classifier on TCGA DNA Methylation Data

The HM450 methylation data for the prostate adenocarcinoma samples from TCGA project was downloaded from the TCGA Data Portal (https://tcga-data.nci.nih.gov/tcga/). After filtering samples based on the same criteria as for our own samples, 499 samples (45 normal, 453 tumor, and 1 metastatic) remained. After removing samples with missing values among the 25 predictor probes, 496 samples remained (45 normal, 450 tumor, and 1 metastatic). The classifier was run on these samples and 70% were predicted with a probability above a cutoff of 0.67. A cutoff of 0.67 was chosen because as a consequence the probability for either of the two other groups must be 0.33 or less. Clinicopathological data was available for most samples in Biotab-files and are shown for the samples predicted above the 0.67 cutoff.

### Statistics

In FIG. 5A and FIG. 5B, Welch two sample t-tests were used to calculate statistical significance. In FIG. 5C, FIG. 5D and FIG. 5E, Fisher's exact 2-tailed tests were used to calculate significance. P-values<0.05 were considered significant.

### Data Access

Methylation array data have been submitted to the NCBI Gene Expression Obnibus (GEO; www.ncbi.nlm.nih.gov/geo/) under accession number GSE73549.

Identification of PC aggressiveness is fundamental to improving clinical decision-making in patients diagnosed with organ-confined PC regarding treatment or active surveillance. By implementing our design of examining DNA methylation in primary multifocal PC and matched lymph node metastases, we were able to examine the relationships amongst primary foci as well as the relationships between primary foci and metastases. Importantly, we found that more than half of the patients in our cohort showed multiple subclones, findings similar to previously reported studies^{9,11-14,16-18}, and also that DNA methylation of a lymph node metastasis is similar to a cancerous focus/foci from the same patient. Taking advantage of these findings, we developed a method to categorize the subclonal relationship and aggressiveness of individual PC foci. The resulting aggressive and non-aggressive sample groups, along with adjacent-normal samples, were used to search for biomarkers to distinguish the three groups, and the outcome was a 25-probe aggressiveness classifier. The classifier showed prognostic value when it was applied to samples from the PC cohort from TCGA.

For this study, we relied on the assumption that DNA methylation can inform on clonal evolution. Several studies have addressed the connection between DNA methylation and clonal evolution with high precision^{11,27,28} and recently, Costello and colleagues reported that phyloepigenetic relationships robustly recapitulate phylogenetic patterns in gliomas and their recurrences²⁹. Two or more foci originated from the same subclone in 11 of 14 patients in our cohort (FIG. 3A), indicating that an initial subclone seeded multiple locations through migration. We cannot definitively rule out that these are not actually one large or branched focus, since a fine physical connection can be hard to clearly distinguish in a pathological sample. Therefore, we do not attempt to determine which focus from the same subclone gave rise to the PL.

While clinical tools and techniques have improved immensely^{2,4,36-39}, the determination of tumor aggressiveness prior to physical manifestation must rely on biomarkers measured biochemically or at a molecular level. One impediment to success is how to define tumor aggressiveness with respect to a clinical end point. Often GS or time to PSA recurrence is used as a surrogate for PC aggressiveness, which would be more appropriately evaluated using metastatic progression or mortality. In this study, we used a novel approach in defining aggressiveness as the ability to give rise to lymph node metastases. The presence of lymph node metastases is an indication of tumor cells having acquired the ability to leave the primary site and proliferate in a secondary site, and thus acts as an indicator for the capacity of the cancer to establish distant metastases. In addition to this type of lymphatic dissemination, metastases can also arise through hematogenous dissemination to brain, lungs, liver, and bone marrow⁴⁸. Secondary cancer growths at these sites are not routinely removed during treatment for metastatic PC, and thus, the tissue for research is not available until postmortem. Although we recognize that distant metastases do not exclusively arise through lymphatic dissemination, we show that this clinical end point is very relevant alone or in concert with other clinicopathological parameters (FIG. 4A-4C, FIG. 5A-5E).

Gleason score 7 (GS 7) tumors are among the most difficult and poorly established backgrounds for making clinical decisions^{49,50}, however, our study demonstrated that aggressiveness of PCs with GS 7 using our classifier is highly correlated with pathological tumor stage, but not specific for primary or secondary Gleason patterns (4+3 or 3+4, FIG. 5B). Because of this, our classifier may challenge the current standard for clinical care, and may result in placing select PC patients into active surveillance and avoid invasive treatments.

A limitation to the presented study is that our discovery set is effectively only 14 patients, from whom we have 79 total samples. A larger discovery set would improve the study and would probably result in an enlargement of the classifier to more than 25 probes due to the vast PC heterogeneity⁵¹. Despite the modest size of the discovery set, we were able to validate the aggressiveness classifier, and thus, our study approach using publicly available TCGA prostate adenocarcinoma DNA methylation data from 496 primary tissues. Upon correlating our predictions with the TCGA clinicopathological information, we found a significant association (P<0.02) between aggressiveness and pre-operative PSA levels, pathological GS, presence of lymph node metastases and tumor stage, but interestingly, we did not find any correlation with tumor size. We do recognize that different clinical endpoints would be better suited to describe poor clinical outcome, however, but regret that the average follow-up period of the TCGA PRAD cohort was only 3.16 years. As a result, we found that too few patients had recurred and thus only found a significant difference between the groups for tumor status. Taken together, the presented data indicate the importance of using DNA methylation data to identify aggressive lesions more specifically than any currently used approach. This approach has clinical applications for early detection in PC biopsy specimens.

Upon suspicion of PC, prostate biopsies are performed as the standard-of-care method for PC diagnosis⁴⁰. Currently, prostate needle biopsies are most commonly performed trans-rectally in a systematic, yet random format. This systematic, random biopsy strategy has a high rate of misdiagnoses, since the non-targeted needles may either miss the clinically significant cancer focus, capture only a clinically insignificant cancer focus, or completely miss all cancer foci^{20,41}. Thus, the significant sampling error of traditional systematic, random prostate biopsies renders them unreliable for accurate characterization of index tumor location, volume, and GS⁴¹. The recently developed image-guided targeted prostate biopsy technique, which fuses magnetic resonance and three-dimensional transrectal ultrasound images, can reliably identify the location and the primary Gleason pattern of index lesions^{38,39}. By combining image-guided targeted biopsies and our DNA methylation classifier (following further clinical validation), the ability to identify aggressive focus, and subsequently characterize biopsy-detected PC foci more accurately should be enhanced. The ability to determine aggressiveness in a biopsy sample mapped to a particular prostate location should lead to the ability to make more informed clinical decisions regarding the choice between active surveillance of non-aggressive PC foci and surgery or targeted focal ablation therapy of the aggressive PC foci. Initially, the aggressiveness classifier should be developed into a more cost- and labor efficient test in the form of a custom DNA methylation array or multiplexed PCR-based assay (MSP or MethyLight)^{42,52}.

TABLE 4
Sequences of Probes

| | | | | RELATION_TO_ | | INFINIUM_ |
|---|---|---|---|---|---|---|
| | | UCSC_REFGENE_ | UCSC_REFGENE_ | UCSC_CPG_ | | DESIGN_ |
| c | TargetID | NAME | GROUP | ISLAND | ENHANCER | TYPE |
| 1 | cg00017221 | | | Island | NA | I |
| 2 | cg00018845 | | | Island | NA | II |
| 3 | cg00021256 | FEV | Body | N_Shore | TRUE | II |
| 4 | cg00036011 | HIST1H3G; HIST1H2 | TSS200; TSS1500 | N_Shore | NA | II |
| | | BI | | | | |
| 5 | cg00046499 | GPR149 | Body | N_Shore | TRUE | II |
| 6 | cg00055529 | FBX047 | TSS200 | | NA | II |
| 7 | cg00063174 | WDR69 | TSS200 | Island | TRUE | I |
| 8 | cg00074145 | ONECUT1 | Body | N_Shore | NA | I |
| 9 | cg00083018 | | | N_Shore | NA | II |
| 10 | cg00087368 | SIM1 | Body | N_Shore | TRUE | II |
| 11 | cg00088183 | | | Island | NA | II |
| 12 | cg00107682 | | | Island | NA | II |
| 13 | cg00107772 | | | Island | NA | II |
| 14 | cg00114913 | LONRF3; LONRF3 | TSS1500; TSS1500 | Island | NA | I |
| 15 | cg00116265 | ZNF673; ZNF673; ZNF | TSS1500; TSS1500 | N_Shore | NA | II |
| | | 63; ZNF673 | ; TSS1500; TSS150 | | | |
| | | | 0 | | | |
| 16 | cg00121904 | MAOB | TSS200 | Island | NA | II |
| 17 | cg00152008 | DMRTA2 | TSS200 | Island | NA | I |
| 18 | cg00153106 | | | Island | TRUE | II |
| 19 | cg00154357 | ZIC4; ZIC4; ZIC4; ZIC4 | Body; TSS1500; Bo | Island | NA | I |
| | | ; ZIC4 | dy; Body; TSS1500 | | | |
| 20 | cg00154455 | ZNF5I7 | TSS1500 | N_Shore | NA | II |
| 21 | cg00155526 | NPR3 | Body | Island | NA | II |
| 22 | cg00158308 | OXGR1 | 5′UTR | N_Shore | TRUE | II |
| 23 | cg00170003 | NR2E1 | Body | Island | TRUE | II |
| 24 | cg00178984 | SLC16A7 | 5′UTR | | NA | I |
| 25 | cg00187981 | | | Island | NA | II |
| 26 | cg00188409 | | | N_Shore | TRUE | II |
| 27 | cg00192136 | DBX1 | Body | Island | NA | II |
| 28 | cg00209038 | LHCGR | 1stExon | Island | TRUE | I |
| 29 | cg00210994 | C3orf26; FILIP1L; MIR | Body; Body; Body; 5 | N_Shore | NA | II |
| | | 548G; FILIP1L; FILIP1 | ′UTR; Body | | | |
| | | L | | | | |
| 30 | cg00224028 | SEMA3D | Body | | NA | II |
| 31 | cg00243661 | OR2D2 | 1stExon | | NA | II |
| 32 | cg00262621 | MMP23B | TSS1500 | Island | TRUE | II |
| 33 | cg00268840 | ALX4 | TSS1500 | Island | NA | I |
| 34 | cg00287829 | GRIN2A; GRIN2A; GR | TSS1500; 5′UTR; T | Island | NA | I |
| | | IN2A | SS200 | | | |
| 35 | cg00296121 | FRMD4A | Body | N_Shore | NA | II |
| 36 | cg00326231 | CLTCLI; CLTCLI | Body; Body | N_Shore | NA | II |
| 37 | cg00329697 | PTCH2 | 3′UTR | | TRUE | I |
| 38 | cg00332937 | | | Island | NA | I |
| 39 | cg00338801 | | | S_Shore | TRUE | II |
| 40 | cg00347563 | GIPC2; GIPC2 | 1stExon; 5′UTR | Island | NA | I |
| 41 | cg00348762 | DKFZP434HI68; GNA | Body; Body; Body | S_Shore | NA | II |
| | | O1; GNAO1 | | | | |
| 42 | cg00352218 | | | N_Shore | NA | II |
| 43 | cg00360610 | SNRNP200 | Body | N_Shore | NA | II |
| 44 | cg00369477 | HTR2C; HTR2C | 5′UTR; 1stExon | Island | TRUE | II |
| 45 | cg00369811 | | | Island | TRUE | I |
| 46 | cg00374016 | | | Island | NA | II |
| 47 | cg00377070 | PLXNA4 | 3′UTR | | NA | II |
| 48 | cg00379648 | RFX4 | Body | Island | TRUE | I |
| 49 | cg00380985 | TCEAL6 | 5′UTR | N_Shore | NA | I |
| 50 | cg00400448 | | | N_Shore | TRUE | I |
| 51 | cg00407329 | SIM1 | Body | N_Shore | NA | II |
| 52 | cg00422120 | FEV | Body | N_Shore | TRUE | II |
| 53 | cg00423153 | SPHKAP; SPHKAP | TSS1500; TSS1500 | S_Shore | NA | I |
| 54 | cg00433861 | DOK6 | Body | Island | NA | II |
| 55 | cg00436476 | HIST1H4F | TSS200 | N _Shore | NA | I |
| 56 | cg00444390 | OR10A6 | TSS200 | | NA | II |
| 57 | cg00463631 | GRIA2; GRIA2; GRIA2 | Body; Body; Body | Island | NA | I |
| 58 | cg00465476 | DUOX2; DUOXA2 | TSS200; TSS200 | Island | NA | I |
| 59 | cg00466108 | DNAJC6 | Body | Island | TRUE | I |
| 60 | cg00474723 | ME3; ME3; ME3; ME3 | TSS200; TSS200; 1s | Island | NA | I |
| | | | tExon; 5′UTR | | | |
| 61 | cg00478361 | | | Island | NA | II |
| 62 | cg00507135 | ACBD5; ACBD5; ACB | Body; Body; Body | N_Shore | NA | II |
| | | D5 | | | | |
| 63 | cg00514609 | CRYAB; HSPB2 | TSS1500; TSS200 | | TRUE | I |
| 64 | cg00527440 | THAP4; THAP4 | Body; Body | Island | NA | II |
| 65 | cg00538458 | | | Island | NA | II |
| 66 | cg00554969 | TBX22; TBX22; TBX2 | Body; 5′UTR; Body | | NA | I |
| | | 2 | | | | |
| 67 | cg00574740 | EM07 | Body | Island | NA | II |
| 68 | cg00594129 | EBF1 | Body | Island | TRUE | II |
| 69 | cg00610991 | SLCSA1 | 5′UTR | N_Shore | NA | II |
| 70 | cg00613562 | SULF1; SULF1; SULF1 | 5′UTR; 1stExon; 1st | | TRUE | II |
| | | ; SULF1 | Exon; 5′UTR | | | |
| 71 | cg00619126 | | | Island | NA | I |
| 72 | cg00625334 | C18orf34; C18orf34 | TSS200; TSS1500 | Island | NA | I |
| 73 | cg00626702 | FAM53A | Body | S_Shore | TRUE | I |
| 74 | cg00641409 | FEJ22536 | Body | Island | NA | I |
| 75 | cg00646019 | | | Island | NA | I |
| 76 | cg00653387 | PTN; PTN | 5′UTR; 1stExon | | NA | II |
| 77 | cg00663077 | ZFP42 | TSS1500 | Island | NA | I |
| 78 | cg00667647 | | | N_Shore | TRUE | II |
| 79 | cg00675569 | ALOX5 | Body | Island | TRUE | I |
| 80 | cg00688681 | | | Island | NA | I |
| 81 | cg00697992 | NXPH2 | Body | N_Shore | TRUE | II |
| 82 | cg00702719 | | | Island | NA | II |
| 83 | cg00739582 | MIR888; MIR890 | TSS1500; TSS1500 | | NA | II |
| 84 | cg00741609 | TEX1 | Body | Island | TRUE | II |
| 85 | cg00741836 | DOK5 | TSS200 | Island | NA | I |
| 86 | cg00744866 | CLASP2 | Body | | TRUE | II |
| 87 | cg00748427 | | | | TRUE | II |
| 88 | cg00753399 | MOBKL1B | Body | N_Shore | NA | II |
| 89 | cg00760950 | | | | TRUE | II |
| 90 | cg00765312 | ALX1 | Body | Island | NA | I |
| 91 | cg00766845 | NID2 | TSS200 | Island | NA | II |
| 92 | cg00767395 | | | N_Shore | NA | II |
| 93 | cg00772407 | DUSP6; DUSP6 | Body; Body | N_Shore | NA | II |
| 94 | cg00789198 | | | | NA | II |
| 95 | cg00802478 | RBM47 | 5′UTR | | TRUE | II |
| 96 | cg00817100 | | | Island | NA | I |
| 97 | cg00822361 | SSR3 | Body | N_Shore | NA | II |
| 98 | cg00838898 | | | N_Shore | TRUE | II |
| 99 | cg00839579 | MEOX2 | TSS1500 | | TRUE | I |
| 100 | cg00840332 | LEP | TSS200 | Island | NA | I |
| 101 | cg00850552 | SUM03 | Body | N_Shore | NA | II |
| 102 | cg00854166 | EPO | Body | S_Shore | TRUE | II |
| 103 | cg00862116 | ABCB1 | 5′UTR | S_Shore | NA | II |
| 104 | cg00864458 | RPS20; RPS20 | Body; 3′UTR | N_Shore | NA | II |
| 105 | cg00875272 | TAL1; TAL1 | 5′UTR; 1stExon | N_Shore | NA | II |
| 106 | cg00880452 | SYCN | 1stExon | Island | NA | I |
| 107 | cg00884221 | IGFBP7 | TSS1500 | Island | NA | II |
| 108 | cg00893471 | HOXD3 | 5′UTR | Island | NA | II |
| 109 | cg00910015 | | | | NA | I |
| 110 | cg00918522 | | | N_Shelf | TRUE | II |
| 111 | cg00921266 | HOXA3; HOXA3; HO | 5′UTR; 5′UTR; TSS | N_Shore | NA | I |
| | | XA3 | 200 | | | |
| 112 | cg00926215 | PAX3; PAX3; PAX3; C | Body; Body; Body; T | N_Shore | NA | I |
| | | CDC140; PAX3; PAX3; | SS1500; Body; Bod | | | |
| | | PAX3; PAX3; PAX3 | y; Body; Body; Body | | | |
| 113 | cg00928167 | | | Island | TRUE | II |
| 114 | cg00930873 | ALDH1A2; ALDH1A2 | TSS200; TSS200 | Island | TRUE | II |
| 115 | cg00932276 | EGFL6; EGFL6; EGFL | 5′UTR; 1stExon; 5′U | Island | TRUE | II |
| | | 6; EGFL6 | TR; 1stExon | | | |
| 116 | cg00944193 | NBEA | Body | Island | NA | I |
| 117 | cg00947032 | SPG20; SPG20; SPG20; | 5′UTR; 5′UTR; 5′UT | Island | NA | II |
| | | SPG20 | R; 5′UTR | | | |
| 118 | cg00963378 | GJA3 | Body | Island | NA | I |
| 119 | cg00971050 | PTRF | Body | Island | NA | I |
| 120 | cg00980904 | RASSF1; RASSF1; RA | Body; Body; Body; 5 | N_Shore | NA | II |
| | | SSF1; RASSF1 | ′UTR | | | |
| 121 | cg00990022 | NT5C1A | TSS1500 | Island | TRUE | II |
| 122 | cg00996827 | | | | NA | I |
| 123 | cg01016662 | | | Island | TRUE | I |
| 124 | cg01019587 | | | Island | NA | II |
| 125 | cg01025095 | | | | NA | I |
| 126 | cg01035198 | C6orf141; C6orf141 | 1stExon; 3′UTR | S_Shore | TRUE | II |
| 127 | cg01042334 | OPLAH | 5′UTR | Island | TRUE | II |
| 128 | cg01042641 | IFT140 | Body | S_Shore | NA | I |
| 129 | cg01056242 | GPR133 | Body | N_Shore | NA | I |
| 130 | cg01063813 | STAT6; STAT6 | 1stExon; 5′UTR | | TRUE | II |
| 131 | cg01065210 | RER1 | 3′UTR | S_Shore | NA | II |
| 132 | cg01068023 | | | N_Shore | NA | II |
| 133 | cg01085837 | | | Island | NA | II |
| 134 | cg01088410 | | | Island | NA | I |
| 135 | cg01098812 | | | Island | TRUE | II |
| 136 | cg01120165 | HDAC10; HDAC10 | TSS1500; TSS1500 | Island | NA | I |
| 137 | cg01128642 | OSR1 | TSS1500 | S_Shore | NA | I |
| 138 | cg01135546 | | | Island | NA | I |
| 139 | cg01138867 | GABRB2; GABRB2 | TSS1500; TSS1500 | S_Shore | NA | I |
| 140 | cg01167755 | NRG2; NRG2; NRG2; N | Body; Body; Body; | S_Shore | NA | I |
| | | RG2 | Body | | | |
| 141 | cg01175020 | TEX1 | Body | Island | NA | I |
| 142 | cg01186595 | CHORDC1; CHORDC | Body; Body | N_Shore | NA | II |
| | | 1 | | | | |
| 143 | cg01203385 | C2orf43 | TSS200 | Island | NA | I |
| 144 | cg01209023 | FTH1 | Body | N_Shore | NA | II |
| 145 | cg01218619 | | | Island | TRUE | I |
| 146 | cg01221209 | | | | NA | I |
| 147 | cg01245966 | ADCY8 | TSS1500 | Island | NA | II |
| 148 | cg01258751 | SIM1 | TSS1500 | N_Shore | TRUE | II |
| 149 | cg01272707 | | | Island | NA | I |
| 150 | cg01291336 | SMARCD3; SMARCD | Body; Body | S_Shore | TRUE | II |
| | | 3 | | | | |
| 151 | cg01308827 | ETV2 | TSS200 | N_Shore | NA | I |
| 152 | cg01309153 | SURF1; SURF2 | TSS1500; Body | S_Shore | NA | II |
| 153 | cg01348733 | SLFN11; SEFN11; SLF | TSS200; TSS200; T | Island | TRUE | I |
| | | N1; SLFN11; SLFN11 | SS200; TSS200; TS | | | |
| | | | S200 | | | |
| 154 | cg01384143 | C3orf14 | TSS1500 | Island | NA | I |
| 155 | cg01386424 | | | Island | NA | II |
| 156 | cg01386978 | PARK2; PARK2; PAR | Body; Body; Body | S_Shore | NA | I |
| | | K2 | | | | |
| 157 | cg01404317 | SPG20; SPG20; SPG20; | 5′UTR; 5′UTR; 5′UT | Island | NA | II |
| | | SPG20 | R; 5′UTR | | | |
| 158 | cg01410316 | ADAP1 | Body | N_Shore | NA | II |
| 159 | cg01413054 | CAB39; CAB39; CAB3 | Body; Body; Body | | NA | II |
| | | 9 | | | | |
| 160 | cg01419831 | | | Island | TRUE | I |
| 161 | cg01419991 | TRIB1 | 3′UTR | | NA | II |
| 162 | cg01422797 | PRDMI3 | TSS1500 | N_Shore | NA | II |
| 163 | cg01439876 | | | Island | TRUE | I |
| 164 | cg01443071 | CNP | Body | Island | NA | I |
| 165 | cg01476568 | C10orf18 | 5′UTR | | TRUE | I |
| 166 | cg01481159 | | | Island | NA | II |
| 167 | cg01496199 | | | Island | TRUE | I |
| 168 | cg01498231 | SPOCK2; SPOCK2 | Body; Body | Island | NA | I |
| 169 | cg01498900 | ACOT2 | TSS200 | N_Shore | NA | II |
| 170 | cg01503299 | NOPI4 | Body | N_Shore | NA | I |
| 171 | cg01505767 | LOC284798; LOC2847 | Body; TSS1500; Bo | Island | TRUE | I |
| | | 98; LOC284798 | dy | | | |
| 172 | cg01520122 | CCL7 | TSS1500 | | NA | II |
| 173 | cg01521258 | CACHD1 | Body | S_Shore | NA | II |
| 174 | cg01533258 | GSC | Body | Island | TRUE | I |
| 175 | cg01546378 | GALM | TSS200 | | NA | II |
| 176 | cg01548300 | | | Island | NA | II |
| 177 | cg01563704 | HIST1H3G; HIST1H2 | TSS1500; TSS1500 | N_Shore | NA | II |
| | | B1 | | | | |
| 178 | cg01564165 | | | | NA | II |
| 179 | cg01587682 | PAX6; PAX6; PAX6 | Body; Body; Body | Island | TRUE | II |
| 180 | cg01589129 | | | Island | TRUE | I |
| 181 | cg01615475 | | | Island | NA | I |
| 182 | cg01625087 | | | N_Shore | NA | I |
| 183 | cg01635555 | | | S_Shore | NA | II |
| 184 | cg01657186 | NINJ2 | Body | S_Shore | TRUE | I |
| 185 | cg01661350 | KLHL1; KLHL1; ATX | 1stExon; 5′UTR; Bo | Island | TRUE | II |
| | | N8OS | dy | | | |
| 186 | cg01663018 | | | Island | NA | I |
| 187 | cg01697719 | TBX1; TBX1; TBX1 | Body; Body; Body | Island | TRUE | I |
| 188 | cg01709619 | | | S_Shore | NA | I |
| 189 | cg01718742 | CDH8 | TSS200 | Island | NA | I |
| 190 | cg01735384 | | | Island | NA | I |
| 191 | cg01737550 | TCEAL6 | 3′UTR | N_Shelf | NA | II |
| 192 | cg01738650 | | | | TRUE | II |
| 193 | cg01751605 | PTN | TSS200 | | NA | II |
| 194 | cg01754780 | SRCIN1 | Body | Island | TRUE | I |
| 195 | cg01758418 | SLC7A3; SLC7A3 | TSS1500; TSS1500 | S_Shore | NA | II |
| 196 | cg01758512 | FUT9; FUT9 | 5′UTR; 1stExon | Island | NA | I |
| 197 | cg01759597 | DLX6AS | Body | | NA | I |
| 198 | cg01765758 | | | Island | NA | II |
| 199 | cg01765882 | LOC116437 | Body | | NA | I |
| 200 | cg01779765 | FLJ43390 | TSS1500 | Island | NA | I |
| 201 | cg01789478 | NEUROD1 | TSS200 | N_Shelf | TRUE | I |
| 202 | cg01796225 | CHORDC1; CHORDC | Body; Body | N_Shore | NA | II |
| | | 1 | | | | |
| 203 | cg01804193 | | | Island | TRUE | II |
| 204 | cg01805282 | EYA4; EYA4; EYA4 | 5′UTR; 5′UTR; 5′UT | Island | TRUE | II |
| | | | R | | | |
| 205 | cg01810111 | | | | TRUE | II |
| 206 | cg01814945 | HEA-J; NCRNA00171 | Body; Body | Island | TRUE | 1 |
| 207 | cg01816515 | C2orf43 | 5′UTR | Island | NA | II |
| 208 | cg01819167 | | | | NA | II |
| 209 | cg01819759 | RNF219 | TSS1500 | S_Shore | NA | II |
| 210 | cg01832005 | C3orf14 | TSS1500 | Island | NA | II |
| 211 | cg01832036 | DEX4; DLX4; DEX4 | Body; 5′UTR; 1stEx | Island | NA | I |
| | | | on | | | |
| 212 | cg01837719 | FUT9 | TSS1500 | N_Shore | NA | II |
| 213 | cg01841641 | | | N_Shore | NA | I |
| 214 | cg01842807 | CYP2W1 | Body | Island | NA | I |
| 215 | cg01851874 | RUNX1T1; RUNX1T1; | Body; 5′UTR; 1stEx | | TRUE | I |
| | | RUNX1T1; RUNX1T1 | on ; 5′UTR | | | |
| 216 | cg01866955 | | | Island | NA | I |
| 217 | cg01871127 | | | N_Shore | NA | II |
| 218 | cg01872216 | | | Island | TRUE | I |
| 219 | cg01880908 | TNRC6C; TNRC6C | 3′UTR; 3′UTR | Island | NA | I |
| 220 | cg01883966 | STK19; STK19; STK19 | TSS200; TSS1500; | N_Shore | NA | II |
| | | ; DOM3Z | TSS1500; Body | | | |
| 221 | cg01906055 | NCAPH | TSS1500 | N_Shore | NA | II |
| 222 | cg01912955 | SIM1 | Body | Island | NA | II |
| 223 | cg01913196 | DCAF12L1 | TSS200 | S_Shore | NA | I |
| 224 | cg01916115 | LAMA2; LAMA2; LA | 5′UTR; 1stExon; 1st | | TRUE | II |
| | | MA2; LAMA2 | Exon; 5′UTR | | | |
| 225 | cg01932956 | | | Island | NA | I |
| 226 | cg01940855 | CHST11 | Body | Island | NA | I |
| 227 | cg01942318 | | | | TRUE | II |
| 228 | cg01957088 | HAS2; HAS2AS | 5′UTR; TSS200 | N_Shore | NA | II |
| 229 | cg01958086 | MEIS2; MEIS2; MEIS2 | Body; Body; Body; | N_Shore | NA | II |
| | | ; MEIS2; MEIS2; MEIS | Body Body Body; | | | |
| | | 2; MEIS2 | Body | | | |
| 230 | cg01968493 | ZNF804A | TSS1500 | N_Shore | NA | II |
| 231 | cg01972576 | GML; GML | 5′UTR; 1stExon | Island | NA | I |
| 232 | cg01978237 | SIM1 | Body | N_Shore | NA | II |
| 233 | cg01981601 | TOX | TSS1500 | Island | NA | I |
| 234 | cg01984743 | STC2 | Body | N_Shelf | TRUE | I |
| 235 | cg01987516 | | | Island | NA | II |
| 236 | cg01996723 | | | N_Shore | NA | II |
| 237 | cg01997272 | | | Island | TRUE | I |
| 238 | cg01999333 | CASP14 | TSS1500 | | NA | II |
| 239 | cg02009088 | NRG2; NRG2; NRG2; N | Body; Body; Body; | Island | NA | I |
| | | RG2 | Body | | | |
| 240 | cg02025583 | TMEM220 | Body | Island | NA | II |
| 241 | cg02026062 | FAM162B | Body | Island | NA | I |
| 242 | cg02029926 | CXCL1 | TSS1500 | N_Shore | NA | II |
| 243 | cg02053896 | SLC7A3; SLC7A3 | TSS200; TSS200 | S_Shore | NA | II |
| 244 | cg02056062 | WWTR1; WWTR1; W | Body; Body; Body | Island | NA | II |
| | | WTR1 | | | | |
| 245 | cg02058408 | | | Island | NA | I |
| 246 | cg02078690 | BSX | TSS200 | Island | TRUE | I |
| 247 | cg02081266 | | | Island | TRUE | I |
| 248 | cg02086467 | OTX2 | 5′UTR | Island | NA | I |
| 249 | cg02099878 | | | N_Shore | NA | I |
| 250 | cg02100674 | NRG3; NRG3; NRG3 | Body; Body; TSS15 | S_Shore | NA | I |
| | | | 00 | | | |
| 251 | cg02120658 | PAX3; PAX3; PAX3; P | Body; Body; Body; | Island | TRUE | I |
| | | AX3; PAX3; PAX3; PA | Body; Body; Body; | | | |
| | | X3; PAX3 | Body; Body | | | |
| 252 | cg02134353 | | | Island | NA | II |
| 253 | cg02142896 | | | | TRUE | II |
| 254 | cg02144933 | AOX1 | TSS200 | Island | TRUE | II |
| 255 | cg02160684 | TRIB1 | Body | S_Shelf | NA | II |
| 256 | cg02167438 | PAK7; PAK7 | TSS200; TSS200 | Island | TRUE | I |
| 257 | cg02179029 | RBM41; RBM41 | Body; Body | N_Shelf | NA | II |
| 258 | cg02179455 | | | N_Shore | NA | II |
| 259 | cg02183671 | CD38; CD38 | 1stExon; 5′UTR | Island | NA | I |
| 260 | cg02192673 | NPFFR2; NPFFR2 | 1stExon; 5′UTR | Island | NA | II |
| 261 | cg02192855 | HIST1H2B1 | TSS200 | N_Shore | NA | II |
| 262 | cg02197293 | FBXO27; FBXO27 | 5′UTR; 1stExon | Island | NA | I |
| 263 | cg02215070 | AKR1B1 | TSS200 | Island | NA | I |
| 264 | cg02231729 | HAPEN3 | 5′UTR | Island | NA | I |
| 265 | cg02233559 | SLC46A3; SLC46A3 | TSS200; TSS200 | Island | NA | II |
| 266 | cg02237629 | | | N_Shore | NA | I |
| 267 | cg02239640 | GSPT2 | 1stExon | S_Shore | NA | II |
| 268 | cg02242344 | | | N_Shore | NA | I |
| 269 | cg02245794 | TACC1; TACC1 | 1stExon; 5′UTR | | TRUE | II |
| 270 | cg02264082 | | | N_Shore | TRUE | I |
| 271 | cg02285155 | | | | TRUE | II |
| 272 | cg02288754 | SALL1; SALL1 | Body; 5′UTR | Island | NA | II |
| 273 | cg02305242 | FLRT2 | TSS1500 | N_Shore | NA | I |
| 274 | cg02305377 | | | N_Shelf | NA | I |
| 275 | cg02366534 | ME1 | TSS200 | Island | NA | II |
| 276 | cg02388453 | C15orf41; C15orf41 | Body; Body | | TRUE | I |
| 277 | cg02391441 | | | N_Shore | NA | II |
| 278 | cg02395819 | | | Island | TRUE | II |
| 279 | cg02415992 | GRIN2A; GRIN2A; GR | Body; Body; Body | | TRUE | II |
| | | IN2A | | | | |
| 280 | cg02430935 | HMX2 | Body | Island | TRUE | I |
| 281 | cg02432280 | | | Island | NA | II |
| 282 | cg02433564 | | | Island | TRUE | I |
| 283 | cg02446647 | EYA4; EYA4; EYA4 | TSS1500; TSS1500 | Island | NA | I |
| | | | ; TSSI500 | | | |
| 284 | cg02447304 | | | Island | NA | I |
| 285 | cg02454806 | ERFN5 | TSS1500 | N_Shore | NA | II |
| 286 | cg02457328 | | | S_Shelf | NA | II |
| 287 | cg02457623 | ATP2B4; ATP2B4 | 5′UTR; 5′UTR | S_Shore | NA | I |
| 288 | cg02460251 | | | Island | NA | I |
| 289 | cg02489958 | NKX6-1 | Body | N_Shore | NA | II |
| 290 | cg02490989 | MTMR7 | TSS1500 | S_Shore | NA | II |
| 291 | cg02493602 | ME3; ME3; ME3 | TSS1500; TSS200; | S_Shore | NA | I |
| | | | TSS1500 | | | |
| 292 | cg02509086 | | | N_Shore | NA | II |
| 293 | cg02523640 | FOX12 | TSS1500 | Island | NA | I |
| 294 | cg02532538 | | | Island | NA | I |
| 295 | cg02533058 | OPRM1; OPRM1; OPR | Body; Body; Body; | S_Shelf | NA | II |
| | | M1; OPRM1; OPRM1; | Body; Body; Body; | | | |
| | | OPRM1; OPRM1; OPR | Body; Body; Body; 5 | | | |
| | | M1; OPRM1; OPRM1; | ′UTR; Body; Body | | | |
| | | OPRM1; OPRM1 | | | | |
| 296 | cg02541778 | | | Island | NA | I |
| 297 | cg02553284 | COL2AI; COL2A1 | Body; Body | N_Shore | NA | I |
| 298 | cg02560085 | PCDHA6; PCDHA2; P | Body; Body; Body; T | N_Shore | NA | II |
| | | CDHA1; PCDHAS ; PC | SS200; Body; Body; | | | |
| | | DHA7; PCDHA1; PCD | Body; Body; TSS20 | | | |
| | | HA6; PCDHA5; PCDH | 0; Body; Body | | | |
| | | AS; PCDHA3; PCDHA | | | | |
| | | 4 | | | | |
| 299 | cg02564134 | ALDH1L1 | TSS1500 | S_Shore | TRUE | I |
| 300 | cg02578368 | HIST1H2BH; HIST1H | TSS1500; 1stExon | Island | NA | I |
| | | 3F | | | | |
| 301 | cg02590060 | C1orf123 | Body | N_Shore | NA | II |
| 302 | cg02621287 | | | Island | NA | I |
| 303 | cg02628016 | KEHL24 | Body | | NA | II |
| 304 | cg02630214 | | | | NA | I |
| 305 | cg02637318 | ADAM32; ADAM32 | 1stExon; 5′UTR | Island | NA | I |
| 306 | cg02644510 | | | S_Shore | NA | I |
| 307 | cg02647878 | RNF175; RNF175 | 5′UTR; 1stExon | Island | NA | I |
| 308 | cg02656891 | C1orf94; C1orf94 | 5′UTR; TSS200 | Island | NA | I |
| 309 | cg02707854 | SLC27A1 | Body | S_Shelf | NA | I |
| 310 | cg02708922 | ADI1 | Body | N_Shore | NA | I |
| 311 | cg02743222 | TLX3; TLX3 | 5′UTR; 1stExon | Island | NA | I |
| 312 | cg02757516 | GPR123 | Body | S_Shelf | NA | II |
| 313 | cg02764478 | SIM1 | Body | N_Shore | NA | II |
| 314 | cg02766845 | | | Island | NA | I |
| 315 | cg02796939 | FOXK2 | Body | N_Shore | NA | I |
| 316 | cg02809746 | | | Island | NA | I |
| 317 | cg02814558 | PRRG3 | Body | S_Shelf | NA | II |
| 318 | cg02818811 | GLIPR1L1 | TSS200 | | NA | I |
| 319 | cg02839824 | | | N_Shore | NA | II |
| 320 | cg02840535 | GTF3C1 | Body | | NA | I |
| 321 | cg02863073 | SYCN | 1stExon | Island | NA | I |
| 322 | cg02874376 | DLK1; DLK1 | 1stExon; 5′UTR | Island | NA | 1 |
| 323 | cg02879662 | HIF3A; HIF3A | Body; TSS1500 | Island | NA | I |
| 324 | cg02883150 | | | | NA | II |
| 325 | cg02883668 | KCNK10 | Body | N_Shore | TRUE | II |
| 326 | cg02888131 | ALDH1A2; ALDH1A2 | TSS200; TSS200 | Island | TRUE | II |
| 327 | cg02908900 | MEOX2 | TSS1500 | | TRUE | I |
| 328 | cg02909790 | HIST1H3G | 1stExon | Island | NA | II |
| 329 | cg02914422 | PDE1C | 5′UTR | Island | NA | I |
| 330 | cg02936263 | CCL7 | TSS1500 | | NA | II |
| 331 | cg02948624 | TBX1; TBX1; TBX1 | TSS1500; TSS1500 | Island | TRUE | II |
| | | | TSS1500 | | | |
| 332 | cg02961934 | | | | NA | I |
| 333 | cg02988312 | KIAA1530 | Body | S_Shore | NA | I |
| 334 | cg03009240 | FZR1 | 5′UTR | S_Shore | NA | I |
| 335 | cg03012726 | | | N_Shelf | NA | I |
| 336 | cg03029734 | | | N_Shore | NA | II |
| 337 | cg03039990 | MIR770; MEG3; MEG3 | TSS1500; Body; Bo | | NA | II |
| | | ; MEG3 | dy; Body | | | |
| 338 | cg03045635 | DRD5 | TSS200 | Island | NA | I |
| 339 | cg03048432 | NIN; NIN; NIN; NIN | 5′UTR; 5′UTR; TSS | | NA | II |
| | | | 1500; 5′UTR | | | |
| 340 | cg03051777 | | | S_Shore | TRUE | II |
| 341 | cg03052128 | | | N_Shelf | NA | II |
| 342 | cg03094728 | AKAP9; AKAP9 | Body; Body | | TRUE | II |
| 343 | cg03097389 | T5C22D1; TSC22D1 | 3′UTR; 3′UTR | N_Shelf | TRUE | II |
| 344 | cg03110310 | ZNF205; ZNF205; MG | Body; Body; Body; | | NA | II |
| | | C3771; MGC3771 | Body | | | |
| 345 | cg03131767 | ABCB9; ABCB9; ABC | 5′UTR; 5′UTR; 5′UT | S_Shore | NA | II |
| | | B9 | R | | | |
| 346 | cg03142256 | | | S_Shore | NA | II |
| 347 | cg03158400 | FAM3B; FAM3B | Body; Body | Island | NA | I |
| 348 | cg03160883 | | | Island | NA | I |
| 349 | cg03161476 | ATP2B4; ATP2B4 | 5′UTR; 5′UTR | N_Shore | NA | II |
| 350 | cg03170013 | C2or85 | TSS200 | N_Shelf | NA | II |
| 351 | cg03171770 | | | Island | NA | I |
| 352 | cg03174043 | | | N_Shore | NA | 11 |
| 353 | cg03175771 | PRR14 | TSS1500 | N_Shore | NA | I |
| 354 | cg03177042 | | | S_Shore | NA | I |
| 355 | cg03177600 | SLITRK3 | 5′UTR | | NA | II |
| 356 | cg03191045 | | | | NA | I |
| 357 | cg03195164 | C3orf37; C3orf37 | Body; Body | S_Shore | NA | II |
| 358 | cg03207574 | PRTFDC1 | Body | Island | NA | I |
| 359 | cg03209557 | HIST1H2BI; HIST1H2 | 3′UTR; 1stExon | S_Shore | NA | II |
| | | BI | | | | |
| 360 | cg03212161 | KIRREL | Body | | TRUE | II |
| 361 | cg03217795 | PRKCB; PRKCB | 1stExon; 1stExon | Island | NA | I |
| 362 | cg03221483 | SEPT9; SEPT9; SEPT9 | 5′UTR; Body; TSS1 | | NA | II |
| | | | 500 | | | |
| 363 | cg03227021 | | | Island | NA | I |
| 364 | cg03240800 | PCDHA6; PCDHA2; P | Body; Body; Body; | N_Shore | NA | II |
| | | CDHA1; PCDHA7; PC | Body; Body; TSS15 | | | |
| | | DHA1; PCDHA5; PCD | 00; Body; Body; TSS | | | |
| | | HA6; PCDHA5; PCDH | 1500; Body; Body | | | |
| | | A5; PCDHA3; PCDHA | | | | |
| | | 4 | | | | |
| 365 | cg03241244 | KDM2B; KDM2B | Body; Body | Island | NA | I |
| 366 | cg03242698 | FLJ41350 | Body | S_Shore | NA | I |
| 367 | cg03257172 | NR5A2; NR5A2 | Body; Body | N_Shore | NA | I |
| 368 | cg03276000 | DPP6; DPP6; DPP6 | Body; Body; Body | | NA | II |
| 369 | cg03278146 | LOC642597 | TSS200 | Island | NA | I |
| 370 | cg03284392 | | | N_Shore | NA | II |
| 371 | cg03290400 | SIM1 | Body | N_Shore | TRUE | II |
| 372 | cg03296575 | | | S_Shelf | NA | II |
| 373 | cg03307401 | KLK13 | Body | Island | NA | I |
| 374 | cg03319638 | PLA2G4D | TSS1500 | | NA | II |
| 375 | cg03321020 | | | | NA | I |
| 376 | cg03330490 | HHEX | Body | Island | NA | I |
| 377 | cg03330678 | SEPT9; SEPT9; SEPT9; | 5′UTR; 5′UTR; Bod | | TRUE | II |
| | | SEPT9 | y; 1stExon | | | |
| 378 | cg03336402 | EXOC4 | Body | | TRUE | II |
| 379 | cg03337918 | PLEKHF2 | 5′UTR | S_Shore | NA | II |
| 380 | cg03347018 | MSC | TSS200 | S_Shore | NA | I |
| 381 | cg03352106 | KCNJ8 | Body | | NA | I |
| 382 | cg03361164 | | | Island | NA | II |
| 383 | cg03362215 | | | S_Shelf | NA | II |
| 384 | cg03367264 | | | | NA | I |
| 385 | cg03388193 | HPSE2; HPSE2; HPSE2 | TSS1500; TSS1500 | S_Shore | NA | II |
| | | ; HPSE2 | ; TSS1500; TSS150 | | | |
| | | | 0 | | | |
| 386 | cg03397307 | EFCAB4B; EFCAB4B; | TSS200; TSS200; T | Island | TRUE | I |
| | | EFCAB4B | SS200 | | | |
| 387 | cg03401357 | | | Island | NA | I |
| 388 | cg03420242 | | | Island | NA | II |
| 389 | cg03423123 | LOC644538 | 1stExon | Island | TRUE | II |
| 390 | cg03440272 | | | Island | TRUE | I |
| 391 | cg03454250 | | | | TRUE | II |
| 392 | cg03463076 | | | | TRUE | II |
| 393 | cg03464366 | | | | NA | II |
| 394 | cg03464514 | PTN; PTN | 5′UTR; 1stExon | | NA | II |
| 395 | cg03465499 | MRGPRF; MRGPRF | TSS1500; TSS1500 | S_Shelf | NA | II |
| 396 | cg03465513 | | | Island | NA | I |
| 397 | cg03469862 | | | Island | NA | I |
| 398 | cg03478497 | C18orf34; C18orf34 | TSS1500; TSS1500 | S_Shore | NA | II |
| 399 | cg03479715 | FLJ43390 | Body | S_Shore | NA | II |
| 400 | cg03480698 | PTPRN2; PTPRN2; PT | Body; Body; Body | S_Shelf | NA | I |
| | | PRN2 | | | | |
| 401 | cg03506640 | FLRT2 | 5′UTR | Island | NA | I |
| 402 | cg03512414 | RIN2 | Body | | TRUE | II |
| 403 | cg03520644 | COL11A1; COL11A1; | TSS1500; TSS1500 | | TRUE | I |
| | | COL11A1; COL11A1 | ; TSS1500; TSS150 | | | |
| | | | 0 | | | |
| 404 | cg03520938 | FAM160B2 | 3′UTR | | NA | I |
| 405 | cg03522150 | C11orf49; C11orf49; C1 | Body; Body; Body; | | TRUE | II |
| | | 1orf49; C11orf49 | Body | | | |
| 406 | cg03533858 | MORN1; RER1 | TSS1500; 5′UTR | S_Shore | NA | II |
| 407 | cg03539016 | | | | NA | I |
| 408 | cg03554749 | | | Island | NA | II |
| 409 | cg03558222 | | | Island | TRUE | II |
| 410 | cg03568017 | SEPT9; SEPT9; SEPT9 | 5′UTR; TSS200; Bo | | TRUE | II |
| | | | dy | | | |
| 411 | cg03572772 | PCDHB15 | TSS1500 | N_Shore | NA | I |
| 412 | cg03583111 | DES | 1stExon | Island | NA | II |
| 413 | cg03584544 | | | | TRUE | II |
| 414 | cg03598731 | | | Island | TRUE | I |
| 415 | cg03602500 | KIR3DX1 | Body | | NA | II |
| 416 | cg03616377 | | | Island | TRUE | II |
| 417 | cg03660500 | LHX9; LHX9 | Body; Body | Island | NA | II |
| 418 | cg03668718 | SLFN11; SLFN11; SLF | TSS1500; TSS1500 | Island | TRUE | I |
| | | N11; SLFN11; SLFN11 | ; TSS1500; TSS150 | | | |
| | | | 0; TSS1500 | | | |
| 419 | cg03671802 | RAX | TSS1500 | Island | NA | II |
| 420 | cg03679755 | | | Island | NA | I |
| 421 | cg03683587 | SHROOM4; SHROOM | TSS200; TSS200 | Island | NA | I |
| | | 4 | | | | |
| 422 | cg03693842 | GABRA3 | TSS1500 | | NA | II |
| 423 | cg03704673 | GLTPD2; GLTPD2 | 5′UTR; 1stExon | Island | NA | I |
| 424 | cg03707308 | NRN1 | Body | Island | NA | I |
| 425 | cg03731268 | L1TD1; L1TD1 | 5′UTR; 5′UTR | Island | NA | I |
| 426 | cg03738384 | COTE1I | Body | | NA | I |
| 427 | cg03752218 | TAL1 | TSS1500 | Island | TRUE | I |
| 428 | cg03758514 | HIST1H2B1 | TSS200 | N_Shore | NA | II |
| 429 | cg03780486 | OR6A2 | TSS200 | | NA | II |
| 430 | cg03784083 | OPCML; OPCML | Body; Body | | TRUE | I |
| 431 | cg03787864 | CYBA | Body | Island | NA | I |
| 432 | cg03790988 | DOK6 | Body | Island | NA | I |
| 433 | cg03804500 | ANAPC2; SSNA1 | Body; TSS1500 | N_Shore | NA | II |
| 434 | cg03815116 | YIPF7 | TSS1500 | | NA | II |
| 435 | cg03830329 | SIM1I | Body | Island | NA | II |
| 436 | cg03857553 | NIPSNAP1 | Body | N_Shore | NA | II |
| 437 | cg03867465 | MGC45800 | Body | Island | NA | I |
| 438 | cg03880173 | SOX20OT | Body | Island | TRUE | II |
| 439 | cg03887163 | LMX1A | Body | N_Shore | NA | II |
| 440 | cg03889013 | | | N_Shore | NA | II |
| 441 | cg03890037 | LVRN | TSS200 | Island | TRUE | II |
| 442 | cg03892838 | LRRF1P1; LRRF1P1; L | TSS1500; TSS1500 | N_Shore | NA | I |
| | | RRF1P1; LRRF1P1; LR | ; TSS1500; TSS150 | | | |
| | | RF1P1 | 0; Body | | | |
| 443 | cg03895540 | OR52N2 | TSS200 | | NA | II |
| 444 | cg03900143 | ZIC4; ZIC4; ZIC4; ZIC4 | Body; TSS1500; Bo | Island | NA | I |
| | | ; ZIC4 | dy; Body; TSS1500 | | | |
| 445 | cg03916909 | | | Island | NA | I |
| 446 | cg03950785 | | | N_Shore | NA | II |
| 447 | cg03951374 | MEIS2; MEIS2; MEIS2 | 1stExon; Body; Bod | Island | NA | II |
| | | ; MEIS2; MEIS2; MEIS | y; 5′UTR; Body; Bod | | | |
| | | 2; MEIS2 | y; Body | | | |
| 448 | cg03953660 | | | Island | TRUE | I |
| 449 | cg03962217 | LOC202781 | Body | S_Shore | NA | II |
| 450 | cg03966514 | SPG20; SPG20; SPG20; | 5′UTR; 5′UTR; 5′UT | Island | NA | I |
| | | SPG20 | R; 5′UTR | | | |
| 451 | cg03981954 | FEZF1; FEZF1 | 3′UTR; 3′UTR | N_Shore | NA | I |
| 452 | cg03985727 | MEOX2 | TSS1500 | | TRUE | II |
| 453 | cg03989125 | | | Island | TRUE | I |
| 454 | cg03999216 | | | Island | NA | II |
| 455 | cg04003327 | ESPNL; SCLY | TSS1500; 3′UTR | N_Shore | TRUE | II |
| 456 | cg04003850 | FGF12; FGF12; FGF12 | 5′UTR; 1stExon; Bo | Island | NA | II |
| | | | dy | | | |
| 457 | cg04007726 | ZMIZ1 | Body | | NA | I |
| 458 | cg04011247 | BCAT1; BCAT1 | 1stExon; 5′UTR | S_Shore | NA | I |
| 459 | cg04025964 | NR5A2; NR5A2 | Body; Body | Island | NA | I |
| 460 | cg04041976 | TRIM55; TRIM55; TRI | Body; Body; Body; | N_Shelf | NA | I |
| | | M55; TRIM55 | Body | | | |
| 461 | cg04052466 | AMR | Body | Island | NA | I |
| 462 | cg04058937 | | | Island | NA | I |
| 463 | cg04066019 | RFTN1 | 5′UTR | Island | NA | I |
| 464 | cg04093225 | ILDR2 | Body | Island | NA | I |
| 465 | cg04098866 | | | Island | NA | I |
| 466 | cg04101806 | AFF3; AFF3 | TSS1500; 5′UTR | S_Shore | TRUE | II |
| 467 | cg04108939 | BEST4 | Body | Island | TRUE | I |
| 468 | cg04111071 | TMEMI96 | TSS1500 | S_Shore | NA | I |
| 469 | cg04120272 | FBXO47 | TSS200 | | NA | II |
| 470 | cg04126266 | PAX7; PAX7; PAX7 | Body; Body; Body | N_Shore | NA | II |
| 471 | cg04131898 | WIT1 | Body | Island | TRUE | I |
| 472 | cg04150495 | MIR663 | TSS1500 | Island | NA | I |
| 473 | cg04151012 | ZNF512 | Body | S_Shore | NA | II |
| 474 | cg04151062 | | | Island | TRUE | II |
| 475 | cg04153740 | | | Island | TRUE | II |
| 476 | cg04156293 | ACHE; ACHE | Body; Body | N_Shore | NA | I |
| 477 | cg04164058 | | | Island | NA | II |
| 478 | cg04174091 | | | S_Shore | NA | II |
| 479 | cg04177426 | | | Island | NA | I |
| 480 | cg04184019 | | | S_Shore | NA | I |
| 481 | cg04194494 | ALOX5 | Body | Island | NA | I |
| 482 | cg04195774 | TNFAIP8 | Body | | TRUE | II |
| 483 | cg04206484 | C7orf50; C7orf50; C7or | Body; Body; Body | S_Shore | TRUE | I |
| | | f50 | | | | |
| 484 | cg04215800 | | | Island | TRUE | II |
| 485 | cg04221650 | ECE4A; LCE4A | 1stExon; 3′UTR | | NA | II |
| 486 | cg04227631 | GABRA3; MIR767; | 5′UTR; TSS1500; T | | NA | II |
| | | MIR105-1 | SS1500 | | | |
| 487 | cg04244354 | | | N_Shore | TRUE | II |
| 488 | cg04248271 | EBF1 | Body | Island | TRUE | I |
| 489 | cg04281464 | | | Island | NA | I |
| 490 | cg04284140 | POLD2; POLD2 | TSS1500; TSS1500 | S_Shore | NA | II |
| 491 | cg04285033 | | | Island | TRUE | I |
| 492 | cg04294888 | TTC28 | Body | | NA | II |
| 493 | cg04299439 | | | N_Shore | NA | II |
| 494 | cg04302300 | | | Island | NA | I |
| 495 | cg04313756 | TCL1A; TCL1A | TSS1500; TSS1500 | Island | NA | I |
| 496 | cg04319276 | | | Island | NA | II |
| 497 | cg04330513 | SFTA3 | TSS1500 | Island | TRUE | I |
| 498 | cg04337056 | GFM1 | TSS1500 | N_Shore | NA | II |
| 499 | cg04343891 | PRDM8; PRDM8 | 5′UTR; 1stExon | S_Shore | NA | I |
| 500 | cg04349243 | SLC16A12 | 5′UTR | Island | TRUE | I |
| 501 | cg04365202 | LHX9; LHX9 | Body; Body | N_Shore | NA | II |
| 502 | cg04380340 | AOX1 | TSS1500 | N_Shore | TRUE | II |
| 503 | cg04388901 | MAP4; MAP4; MAP4 | 5′UTR; 5′UTR; 5′UT | | TRUE | II |
| | | | R | | | |
| 504 | cg04389709 | | | Island | NA | II |
| 505 | cg04399994 | PAK3; PAK3; PAK3 | 5′UTR; 5′UTR; TSS | N_Shore | NA | II |
| | | | 1500 | | | |
| 506 | cg04401986 | FGF5; FGF5 | Body; Body | S_Shelf | NA | I |
| 507 | cg04406436 | | | Island | NA | I |
| 508 | cg04407470 | NR2E1 | Body | Island | TRUE | I |
| 509 | cg04421553 | VILL | Body | S_Shore | NA | II |
| 510 | cg04430582 | MIR195; MIR497 | TSS1500; TSS1500 | N_Shelf | NA | II |
| 511 | cg04452095 | SEPT9; SEPT9; SEPT9; | 5′UTR; 5′UTR; Bod | | TRUE | II |
| | | SEPT9 | y; 1stExon | | | |
| 512 | cg04460984 | CHRNB4 | TSS200 | Island | TRUE | I |
| 513 | cg04461197 | SPATA16 | Body | | TRUE | II |
| 514 | cg04467162 | | | Island | NA | I |
| 515 | cg04467618 | TCF21; TCF21 | 1stExon; 1stExon | Island | NA | II |
| 516 | cg04479219 | SLITRK5 | 5′UTR | Island | NA | II |
| 517 | cg04498418 | GRIK2; GRIK2; GRIK2 | Body; Body; Body | Island | TRUE | II |
| 518 | cg04528545 | | | S_Shore | NA | II |
| 519 | cg04549333 | ALX4 | Body | Island | TRUE | I |
| 520 | cg04550052 | SALL1; SALL1; SALL | Body; 5′UTR; 1stEx | | NA | I |
| | | 1 | on | Island | | |
| 521 | cg04554272 | LMF1 | Body | N_Shore | NA | I |
| 522 | cg04560456 | LIN28B; LIN28B | 1stExon; 5′UTR | S_Shelf | NA | II |
| 523 | cg04560832 | MRPS21; MRPS21 | Body; Body | S_Shore | NA | II |
| 524 | cg04562217 | ROBO1 | 5′UTR | Island | NA | I |
| 525 | cg04567738 | | | N_Shore | TRUE | II |
| 526 | cg04574507 | CD1B | Body | | NA | II |
| 527 | cg04578774 | ALX4 | TSS1500 | Island | NA | I |
| 528 | cg04612444 | PENK; PENK | 5′UTR; TSS200 | Island | NA | I |
| 529 | cg04622802 | FIBIN; FIBIN | 5′UTR; 1stExon | | TRUE | II |
| 530 | cg04628320 | TBCB | TSS1500 | Island | NA | II |
| 531 | cg04634417 | PCDHA6; PCDHA2; P | Body; Body; Body; T | N_Shore | NA | II |
| | | CDHA1; PCDHA5; PC | SS200; Body; Body; | | | |
| | | DHA7; PCDHA1; PCD | Body; Body; TSS20 | | | |
| | | HA6; PCDHA5; PCDH | 0; Body; Body | | | |
| | | AS; PCDHA3; PCDHA | | | | |
| | | 4 | | | | |
| 532 | cg04638468 | TMEM155; LOC10019 | TSS200; Body | Island | NA | I |
| | | 2379 | | | | |
| 533 | cg04640675 | | | Island | NA | I |
| | | | 5′UTR; TSS200; Bo | | | |
| 534 | cg04650676 | SEPT9; SEPT9; SEPT9 | dy | | TRUE | I |
| 535 | cg04652097 | MAGI2 | TSS1500 | Island | NA | I |
| 536 | cg04653522 | RAB38 | TSS200 | S_Shore | NA | II |
| 537 | cg04656042 | | | Island | TRUE | I |
| 538 | cg04660410 | VILL | Body | S_Shore | NA | II |
| 539 | cg04670802 | | | N_Shore | NA | I |
| 540 | cg04684656 | | | Island | NA | II |
| 541 | cg04688351 | PAX3; PAX3; PAX3; P | Body; Body; Body; | N_Shore | NA | I |
| | | AX3; PAX3; PAX3 | Body Body; Body | | | |
| 542 | cg04692403 | TCF2I; TCF21 | 1stExon; 1stExon | Island | NA | II |
| 543 | cg04698114 | SALL1; SALL1; SALL | Body; 5′UTR; 1stEx | Island | NA | I |
| | | 1 | on | | | |
| 544 | cg04701034 | NID2 | TSS1500 | Island | NA | I |
| 545 | cg04722177 | | | Island | TRUE | I |
| 546 | cg04723401 | | | | NA | I |
| 547 | cg04728482 | | | Island | NA | I |
| 548 | cg04738552 | TUBB4 | TSS1500 | S_Shore | NA | I |
| 549 | cg04738965 | ZIC1; ZIC1 | 1stExon; 5′UTR | Island | TRUE | I |
| 550 | cg04761722 | GRIN2A; GRIN2A; GR | 5′UTR; 5′UTR; 5′UT | Island | NA | I |
| | | IN2A | R | | | |
| 551 | cg04771206 | POLR1A; PTCD3 | Body; TSS1500 | N_Shore | NA | II |
| 552 | cg04772683 | KLK10; KLK10; KLK1 | 5′UTR; 5′UTR; 5′UT | Island | NA | I |
| | | 0 | R | | | |
| 553 | cg04804377 | RAD51AP2 | TSS200 | | NA | I |
| 554 | cg04806942 | | | Island | NA | II |
| 555 | cg04828133 | | | Island | NA | 1 |
| 556 | cg04832767 | DEX6AS | Body | N_Shore | NA | I |
| 557 | cg04846243 | IGF2BP1; IGF2BP1; IG | 1stExon; 1stExon; 5′ | Island | NA | I |
| | | F2BP1; IGF2BP1 | UTR; 5′UTR | | | |
| 558 | cg04850731 | NXPH4 | Body | Island | NA | I |
| 559 | cg04851268 | GHSR; GHSR | TSS1500; TSS1500 | N_Shore | TRUE | II |
| 560 | cg04859726 | SIM1 | Body | N_Shore | NA | II |
| 561 | cg04868078 | LHX9; LHX9 | Body; Body | | NA | I |
| 562 | cg04902729 | CACNA1E | TSS200 | Island | NA | II |
| 563 | cg04904331 | VWC2 | TSS1500 | Island | NA | I |
| 564 | cg04910834 | | | | TRUE | II |
| 565 | cg04911050 | RAB40C | 3′UTR | Island | NA | I |
| 566 | cg04927889 | FEZF2 | TSS1500 | S_Shore | NA | I |
| 567 | cg04931126 | | | | TRUE | II |
| 568 | cg04932340 | | | Island | NA | I |
| 569 | cg04935434 | | | Island | NA | I |
| 570 | cg04937184 | BMP4; BMP4 | TSS1500; TSS1500 | S_Shore | NA | I |
| 571 | cg04954680 | LRRC3B | TSS1500 | N_Shore | NA | II |
| 572 | cg04958794 | | | Island | NA | II |
| 573 | cg04961042 | PRKD1 | Body | | TRUE | II |
| 574 | cg04982193 | C8orf33 | Body | S_Shore | NA | II |
| 575 | cg05016408 | LOC134466 | TSS200 | Island | NA | I |
| 576 | cg05019826 | | | | TRUE | I |
| 577 | cg05024627 | | | | NA | II |
| 578 | cg05036656 | | | Island | TRUE | II |
| 579 | cg05042492 | | | N_Shelf | NA | II |
| 580 | cg05049335 | RIN1; RIN1 | 1stExon; 5′UTR | S_Shore | TRUE | II |
| 581 | cg05059304 | SYCP2L | TSS200 | N _Shore | NA | I |
| 582 | cg05060976 | CABYR; CABYR; CA | 3′UTR; 3′UTR; 3′UT | | NA | II |
| | | BYR; CABYR; CABY | R; 3′UTR; 3′UTR; 3′ | | | |
| | | R; CABYR | UTR | | | |
| 583 | cg05079544 | N4BP1 | TSS1500 | S_Shore | NA | II |
| 584 | cg05085500 | | | Island | NA | II |
| 585 | cg05090759 | SLC38A11 | TSS200 | | NA | I |
| 586 | cg05099464 | ARHGEFI2 | Body | | TRUE | II |
| 587 | cg05100070 | SIM1 | Body | N_Shore | NA | II |
| 588 | cg05116382 | ACTN3 | Body | S_Shore | NA | I |
| 589 | cg05119057 | C14orf23 | Body | S_Shore | NA | I |
| 590 | cg05128414 | LHX9 | Body | S_Shore | NA | II |
| 591 | cg05133179 | | | Island | TRUE | I |
| 592 | cg05134041 | POU3F4 | TSS200 | N_Shore | NA | I |
| 593 | cg05135644 | NR2E1 | Body | Island | TRUE | II |
| 594 | cg05137386 | | | N_Shore | NA | I |
| 595 | cg05143887 | ACSS3 | 1stExon | Island | NA | I |
| 596 | cg05156137 | RCAN1; RCAN1; RCA | 5′UTR; Body; 1stEx | | TRUE | II |
| | | N1 | on | | | |
| 597 | cg05159188 | HIST1H4F | TSS200 | N_Shore | NA | II |
| 598 | cg05161795 | ZDHHC3; ZDHHC3; E | TSS1500; TSS1500 | S_Shore | NA | II |
| | | XOSC7; ZDHHC3; EX | ; Body; TSS1500; Bo | | | |
| | | OSC7 | dy | | | |
| 599 | cg05162032 | CYTH3 | Body | N_Shore | NA | II |
| 600 | cg05174942 | VTRNA1-2 | TSS200 | | NA | II |
| 601 | cg05182265 | UBE3C | Body | S_Shore | TRUE | II |
| 602 | cg05200373 | SLC39A7; SLC39A7 | Body; Body | S_Shore | NA | II |
| 603 | cg05210558 | NTM | Body | | NA | II |
| 604 | cg05211057 | COL9A1; COL9A1 | Body; Body | Island | TRUE | II |
| 605 | cg05224998 | SLFN11; SLFN11; SLF | TSS200; TSS200; T | N_Shore | TRUE | I |
| | | N11; SLFN11; SLFN11 | SS200; TSS200; TS | | | |
| | | | S200 | | | |
| 606 | cg05239680 | | | | NA | I |
| 607 | cg05241828 | GPT | TSS1500 | N_Shore | NA | I |
| 608 | cg05247035 | | | | TRUE | II |
| 609 | cg05257528 | PRTN3 | Body | Island | NA | II |
| 610 | cg05270344 | MAGI2 | TSS1500 | Island | NA | II |
| 611 | cg05275752 | GALM | TSS200 | | NA | II |
| 612 | cg05280814 | | | N_Shelf | NA | II |
| 613 | cg05285429 | ROBO2 | Body | | TRUE | II |
| 614 | cg05291374 | DIXDC1; DIXDC1 | TSS1500; Body | N_Shore | NA | II |
| 615 | cg05298458 | TBX1; TBX1; TBX1 | Body; Body; Body | Island | NA | II |
| 616 | cg05298922 | | | Island | NA | II |
| 617 | cg05304531 | DHDDS; HMGN2; DH | 3′UTR; TSS1500; 3′ | N_Shore | TRUE | I |
| | | DDS | UTR | | | |
| 618 | cg05308504 | | | Island | NA | I |
| 619 | cg05314639 | | | | NA | II |
| 620 | cg05327864 | CHRM2; CHRM2; CH | 5′UTR; 5′UTR; 5′UT | Island | TRUE | II |
| | | RM2; CHRM2; CHRM | R; 5′UTR; 5′UTR; 5′ | | | |
| | | 2; CHRM2; CHRM2; C | UTR; 5′UTR; 5′UTR | | | |
| | | HRM2 | | | | |
| 621 | cg05339695 | FLJ43390 | Body | S_Shore | NA | II |
| 622 | cg05345154 | AMH | Body | Island | NA | I |
| 623 | cg05357932 | ZNRF2 | Body | S_Shore | NA | II |
| 624 | cg05372242 | SFTA3 | TSS1500 | Island | TRUE | I |
| 625 | cg05373457 | KCNS2 | 5′UTR | Island | NA | II |
| 626 | cg05376362 | | | | TRUE | II |
| 627 | cg05387146 | ME1 | TSS200 | Island | NA | I |
| 628 | cg05391318 | NR5A2; NR5A2 | Body; Body | Island | NA | I |
| 629 | cg05393297 | | | N_Shore | TRUE | I |
| 630 | cg05400200 | MLL5; MLL5 | Body; Body | | NA | II |
| 631 | cg05414338 | | | N_Shore | NA | II |
| 632 | cg05446860 | LOC284798; LOC2847 | Body; TSS1500; Bo | Island | TRUE | II |
| | | 98; LOC284798 | dy | | | |
| 633 | cg05471509 | | | | NA | II |
| 634 | cg05482942 | | | Island | NA | I |
| 635 | cg05493394 | EYA4; EYA4; EYA4 | TSS1500; TSS1500 | N_Shore | NA | II |
| | | | ; TSS1500 | | | |
| 636 | cg05494014 | MEOX2 | 1stExon | | NA | I |
| 637 | cg05496203 | METS1 | Body | S_Shelf | NA | II |
| 638 | cg05500015 | NAALAD2; NAALAD | 5′UTR; 1stExon | | NA | II |
| | | 2 | | | | |
| 639 | cg05507490 | | | Island | NA | I |
| 640 | cg05525812 | | | N_Shore | NA | I |
| 641 | cg05557266 | DALRD3; DALRD3; N | 5′UTR; TSS1500; T | N_Shore | NA | II |
| | | DUFAF3; NDUFAF3 | SS1500; TSS1500 | | | |
| 642 | cg05560435 | | | Island | TRUE | I |
| 643 | cg05562381 | | | Island | TRUE | I |
| 644 | cg05576949 | IGF2BP2; IGF2BP2 | Body; Body | N_Shore | NA | II |
| 645 | cg05583608 | | | Island | TRUE | I |
| 646 | cg05626117 | CLEC4G | TSS1500 | S_Shore | NA | II |
| 647 | cg05648914 | | | | TRUE | II |
| 648 | cg05658512 | C7orf27 | 5′UTR | N_Shore | NA | II |
| 649 | cg05663573 | SLC7A14 | 5′UTR | N_Shore | NA | I |
| 650 | cg05676622 | RBM4 | Body | Island | NA | I |
| 651 | cg05680973 | HS6ST2; HS6ST2 | Body; Body | N_Shore | NA | II |
| 652 | cg05696715 | ABT1 | Body | Island | NA | II |
| 653 | cg05733181 | | | Island | TRUE | II |
| 654 | cg05735180 | | | Island | NA | I |
| 655 | cg05736424 | | | S_Shelf | NA | II |
| 656 | cg05736768 | DPYS; DPYS | 1stExon; 5′UTR | Island | NA | I |
| 657 | cg05743346 | CALN1; CALN1 | 5′UTR; Body | Island | NA | II |
| 658 | cg05755900 | PFDN6; WDR46; WDR | Body; TSS1500; TS | S_Shore | NA | I |
| | | 46 | S1500 | | | |
| 659 | cg05768044 | CHD9 | Body | | NA | II |
| 660 | cg05769889 | DUSP6; DUSP6 | Body; Body | N_Shore | NA | I |
| 661 | cg05774229 | | | Island | TRUE | I |
| 662 | cg05783185 | EXT1 | Body | N_Shelf | NA | II |
| 663 | cg05790451 | APBA2; APBA2 | Body; Body | Island | TRUE | I |
| 664 | cg05793120 | | | Island | NA | I |
| 665 | cg05803296 | ATXN7L1; ATXN7L1 | TSS200; Body | | TRUE | II |
| 666 | cg05852143 | NTF3 | TSS1500 | Island | NA | I |
| 667 | cg05854114 | | | Island | NA | I |
| 668 | cg05861639 | KIEL28 | 5′UTR | N_Shelf | NA | II |
| 669 | cg05894124 | FGF5; FGF5 | TSS1500; TSS1500 | N_Shore | TRUE | II |
| 670 | cg05901543 | CDH15 | Body | | NA | I |
| 671 | cg05905176 | TNFAIP8L3 | Body | Island | NA | I |
| 672 | cg05907046 | ZSWIM2 | TSS200 | Island | TRUE | II |
| 673 | cg05916684 | | | N_Shore | NA | I |
| 674 | cg05918002 | ZNF232 | 5′UTR | Island | NA | I |
| 675 | cg05920909 | LHX9; LHX9 | Body; Body | N_Shore | TRUE | II |
| 676 | cg05923687 | | | Island | TRUE | I |
| 677 | cg05937630 | AKR7A3 | TSS1500 | Island | NA | I |
| 678 | cg05937737 | SLC7A14 | 5′UTR | Island | NA | I |
| 679 | cg05940231 | TBX15 | TSS200 | Island | TRUE | I |
| 680 | cg05940452 | FOXR1; FOXR1 | 5′UTR; 1stExon | Island | NA | I |
| 681 | cg05944877 | PRKCB; PRKCB | Body; Body | | TRUE | II |
| 682 | cg05945266 | FOXK2 | Body | N_Shore | NA | II |
| 683 | cg05982460 | | | S_Shelf | NA | I |
| 684 | cg05991442 | RECQL5; LOC643008; | Body; Body; 5′UTR | | NA | I |
| | | LOC643008 | | | | |
| 685 | cg05994094 | C18orf34; C18orf34 | TSS200; TSS1500 | Island | NA | I |
| 686 | cg05994320 | | | Island | NA | I |
| 687 | cg06008926 | | | N_Shore | NA | I |
| 688 | cg06014401 | EOMES | TSS1500 | Island | NA | II |
| 689 | cg06022561 | D2HGDH | Body | | NA | I |
| 690 | cg06022664 | MIR548H4 | Body | | NA | I |
| 691 | cg06024295 | CPN1; CPN1 | 1stExon; 5′UTR | | NA | II |
| 692 | cg06025456 | PPT2; PRRT1; PPT2 | TSS1500; TSS1500 | N_Shore | TRUE | I |
| | | | ; TSS1500 | | | |
| 693 | cg06038944 | | | | TRUE | II |
| 694 | cg06057569 | BRD1 | TSS1500 | N_Shore | NA | II |
| 695 | cg06077978 | PCSK1N | Body | Island | NA | I |
| 696 | cg06083330 | POU4F2 | Body | Island | TRUE | I |
| 697 | cg06087421 | TAL1; TAL1 | 5′UTR; 1stExon | N_Shore | NA | I |
| 698 | cg06092815 | SPHKAP; SPHKAP | TSS200; TSS200 | Island | NA | II |
| 699 | cg06108782 | SLC18A3; CHAT; CHA | TSS1500; 1stExon; | Island | NA | II |
| | | T | 5′UTR | | | |
| 700 | cg06120122 | | | | TRUE | II |
| 701 | cg06122635 | | | Island | NA | I |
| 702 | cg06132727 | | | Island | NA | I |
| 703 | cg06148685 | LPAR1; LPAR1 | TSS1500; TSS1500 | Island | NA | II |
| 704 | cg06179765 | GPNMB; GPNMB | Body; Body | Island | TRUE | I |
| 705 | cg06184669 | GABRB1 | Body | N_Shore | TRUE | II |
| 706 | cg06193578 | | | Island | TRUE | II |
| 707 | cg06211837 | ST6GALNAC3; ST6G | 1stExon; 5′UTR; 1st | Island | NA | I |
| | | ALNAC3; ST6GALNA | Exon; 5′UTR | | | |
| | | C3; ST6GALNAC3 | | | | |
| 708 | cg06212263 | | | S_Shore | NA | II |
| 709 | cg06217046 | WWC3 | Body | Island | NA | II |
| 710 | cg06221393 | | | Island | NA | II |
| 711 | cg06240450 | ALDHIL1 | TSS200 | Island | TRUE | I |
| 712 | cg06253058 | WWTR1; WWTR1; W | Body; Body; Body | Island | NA | II |
| | | WTR1 | | | | |
| 713 | cg06254453 | | | N_Shore | NA | II |
| 714 | cg06264868 | | | | NA | II |
| 715 | cg06271630 | | | | NA | II |
| 716 | cg06271970 | | | Island | NA | I |
| 717 | cg06274159 | ZFP42 | TSS200 | Island | NA | II |
| 718 | cg06282524 | | | Island | NA | I |
| 719 | cg06284231 | CLEC14A | 1stExon | Island | NA | II |
| 720 | cg06286796 | PSMD6 | Body | N_Shore | NA | II |
| 721 | cg06288788 | | | S_Shore | NA | II |
| 722 | cg06296503 | | | S_Shore | NA | I |
| 723 | cg06304401 | NLGN1 | 5′UTR | | NA | I |
| 724 | cg06312254 | SLITRK3 | 5′UTR | | NA | II |
| 725 | cg06321998 | | | | NA | II |
| 726 | cg06330323 | TSC2; TSC2; TSC2 | Body; Body; Body | N_Shore | TRUE | I |
| 727 | cg06356912 | VILL | Body | N_Shore | NA | II |
| 728 | cg06369992 | | | Island | NA | II |
| 729 | cg06371502 | | | N_Shore | TRUE | I |
| 730 | cg06385187 | WFDC2 | TSS200 | Island | NA | I |
| 731 | cg06388730 | | | Island | NA | II |
| 732 | cg06401021 | HMGCLL1; HMGCLL | 1stExon; 5′UTR; 1st | Island | TRUE | 1 |
| | | 1; HMGCLL1; HCGCL | Exon; 5′UTR | | | |
| | | L1 | | | | |
| 733 | cg06404175 | OXT | Body | Island | TRUE | I |
| 734 | cg06410057 | | | S_Shore | NA | II |
| 735 | cg06411724 | MEOX2 | Body | | NA | I |
| 736 | cg06424065 | | | Island | NA | I |
| 737 | cg06428055 | ELF4; ELF4 | 5′UTR; 5′UTR | Island | NA | II |
| 738 | cg06439589 | SSR4 | Body | S_Shore | NA | II |
| 739 | cg06444282 | VWA5B1 | Body | Island | NA | I |
| 740 | cg06465306 | ZNF711 | Body | S_Shelf | NA | II |
| 741 | cg06467636 | CNGA2 | 5′UTR | | NA | I |
| 742 | cg06470822 | | | Island | TRUE | II |
| 743 | cg06482019 | | | Island | NA | I |
| 744 | cg06495961 | DPP6; DPP6 | 1stExon; 5′UTR | Island | NA | II |
| 745 | cg06503907 | SYCP2L | TSS200 | N_Shore | NA | I |
| 746 | cg06520675 | FLJ41350 | Body | Island | TRUE | II |
| 747 | cg06580523 | | | N_Shelf | NA | II |
| 748 | cg06583549 | IRF2BP1 | 1stExon | Island | TRUE | I |
| 749 | cg06587521 | LRIG3; LRIG3 | Body; Body | | TRUE | II |
| 750 | cg06598544 | COL9A3 | 3′UTR | Island | NA | I |
| 751 | cg06609094 | | | N_Shelf | TRUE | II |
| 752 | cg06610484 | MCHR2; MCHR2; MC | TSS200; 5′UTR; 1st | Island | TRUE | I |
| | | HR2 | Exon | | | |
| 753 | cg06620896 | | | S_Shore | NA | I |
| 754 | cg06633061 | | | Island | NA | II |
| 755 | cg06645033 | | | Island | NA | I |
| 756 | cg06650546 | | | Island | NA | I |
| 757 | cg06654901 | PAX1 | Body | Island | NA | II |
| 758 | cg06660332 | | | Island | TRUE | I |
| 759 | cg06661450 | LHX9; LHX9 | Body; Body | N_Shore | NA | II |
| 760 | cg06666008 | PAX6; PAX6; PAX6 | Body; Body; Body | N_Shore | NA | I |
| 761 | cg06693048 | EYA1 | TSS1500 | | NA | II |
| 762 | cg06704518 | GATA4 | 5′UTR | Island | NA | II |
| 763 | cg06711380 | FBXO47 | TSS1500 | | NA | II |
| 764 | cg06717750 | RAB40C | Body | N_Shelf | NA | I |
| 765 | cg06720669 | KCNE1L; KCNE1L | 1stExon; 5′UTR | Island | TRUE | I |
| 766 | cg06729532 | | | Island | NA | I |
| 767 | cg06744545 | | | Island | TRUE | I |
| 768 | cg06747432 | CCDC8; CCDC8 | 1stExon; 5′UTR | Island | NA | I |
| 769 | cg06755825 | | | Island | NA | II |
| 770 | cg06759629 | | | Island | TRUE | II |
| 771 | cg06761424 | KIAA1210 | TSS1500 | Island | NA | II |
| 772 | cg06774787 | KLF8; KLF8; KLF8; KL | 5′UTR; 1stExon; 1st | Island | NA | I |
| | | F8 | Exon; 5′UTR | | | |
| 773 | cg06782686 | NID2 | TSS200 | Island | NA | I |
| 774 | cg06793562 | | | Island | NA | II |
| 775 | cg06805513 | SEC16A2 | Body | Island | NA | I |
| 776 | cg06811467 | ATP8B2; ATP8B2 | TSS1500; 5′UTR | N_Shore | NA | II |
| 777 | cg06817490 | | | | TRUE | I |
| 778 | cg06833110 | MEIS1 | Body | Island | NA | I |
| 779 | cg06836736 | ME1 | TSS1500 | Island | NA | II |
| 780 | cg06845853 | WIT1 | Body | S_Shore | NA | I |
| 781 | cg06915321 | ZIC3 | TSS200 | Island | NA | I |
| 782 | cg06915773 | PTN | 5′UTR | | NA | II |
| 783 | cg06916239 | PAX3; PAX3; PAX3; C | Body; Body; Body; 5 | N_Shore | NA | I |
| | | CDC140; CCDC140; P | ′UTR; 1stExon; Bod | | | |
| | | AX3; PAX3; PAX3; PA | y; Body; Body; Body | | | |
| | | X3; PAX3 | ; Body | | | |
| 784 | cg06930722 | CHST11 | Body | | NA | I |
| 785 | cg06943420 | MMP23A; MMP23B | TSS1500; TSS1500 | Island | TRUE | I |
| 786 | cg06943912 | SLC44A2 | Body | S_Shelf | NA | I |
| 787 | cg06960356 | KCNQ1; KCNQ1 | Body; Body | Island | NA | I |
| 788 | cg06962970 | NTM | Body | | TRUE | II |
| 789 | cg06963233 | ANPEP | 5′UTR | Island | NA | II |
| 790 | cg06969287 | TTC28 | Body | | TRUE | II |
| 791 | cg06970190 | | | Island | NA | II |
| 792 | cg06971227 | AATK | Body | Island | NA | II |
| 793 | cg06975914 | | | | TRUE | II |
| 794 | cg06976096 | | | Island | TRUE | II |
| 795 | cg06982190 | | | Island | NA | II |
| 796 | cg06991484 | | | S_Shore | NA | II |
| 797 | cg07017114 | HLA-H | Body | Island | NA | I |
| 798 | cg07019438 | EGEN3 | Body | Island | NA | I |
| 799 | cg07022764 | ASPG | Body | Island | NA | II |
| 800 | cg07034362 | ALDH1L1 | TSS200 | Island | TRUE | I |
| 801 | cg07035145 | PYGL; PYGL | Body; Body | Island | NA | I |
| 802 | cg07037019 | OR10J5 | TSS1500 | | NA | II |
| 803 | cg07037371 | | | | TRUE | I |
| 804 | cg07039423 | KCP; KCP | Body; Body | Island | NA | I |
| 805 | cg07044859 | | | Island | NA | I |
| 806 | cg07045330 | | | Island | NA | II |
| 807 | cg07050616 | FLJ41350 | Body | Island | TRUE | II |
| 808 | cg07053014 | SLC29A1; SLC29A1 | TSS200; TSS200 | N_Shore | NA | II |
| 809 | cg07077013 | | | Island | NA | II |
| 810 | cg07083818 | KIAA1683; KIAA1683 | TSS1500; TSS1500 | | TRUE | II |
| | | ; KIAA1683 | ; TSS1500 | | | |
| 811 | cg07101841 | SEPT9; SEPT9; SEPT9 | 5′UTR; TSS200; Bo | | TRUE | I |
| | | | dy | | | |
| 812 | cg07109801 | MIR425; DALRD3; ND | Body; 5′UTR; TSS1 | N_Shore | NA | II |
| | | UFAF3; NDUFAF3; N | 500; TSS1500; TSS | | | |
| | | DUFAF3; NDUFAF3 | 1500; TSS1500 | | | |
| 813 | cg07137955 | | | Island | TRUE | I |
| 814 | cg07147449 | | | Island | NA | I |
| 815 | cg07173635 | | | Island | NA | II |
| 816 | cg07177380 | | | N_Shelf | TRUE | I |
| 817 | cg07185131 | | | | TRUE | I |
| 818 | cg07190535 | PDE4D; PDE4D | Body; TSS1500 | Island | TRUE | I |
| 819 | cg07197785 | | | Island | TRUE | I |
| 820 | cg07200280 | RFTN1 | 5′UTR | Island | NA | II |
| 821 | cg07211140 | WT1; WT1; WT1; WT1 | Body; Body; Body; | Island | NA | I |
| | | | Body | | | |
| 822 | cg07219542 | GSTM2; GSTM2 | 1stExon; 1stExon | Island | TRUE | I |
| 823 | cg07239592 | POU6F2; POU6F2 | Body; Body | Island | NA | II |
| 824 | cg07262247 | PDLIM4; PDLIM4 | Body; Body | Island | NA | I |
| 825 | cg07271361 | STK19; STK19; DOM3 | Body; Body; TSS15 | S_Shore | NA | II |
| | | Z; STK19 | 00; Body | | | |
| 826 | cg07283795 | | | | TRUE | II |
| 827 | cg07291445 | | | Island | NA | I |
| 828 | cg07302069 | HOXA7; HOXA7 | 5′UTR; 1stExon | Island | NA | I |
| 829 | cg07335294 | SNAP91; SNAP91 | Body; 5′UTR | Island | TRUE | II |
| 830 | cg07349208 | | | Island | TRUE | I |
| 831 | cg07367113 | BRD9; BRD9; BRD9 | Body; Body; Body | N_Shore | NA | II |
| 832 | cg07368796 | | | N_Shore | NA | I |
| 833 | cg07376381 | LCK; LCK | Body; Body | Island | NA | I |
| 834 | cg07378762 | MECOM | Body | N_Shelf | TRUE | II |
| 835 | cg07395354 | MEOX2 | TSS1500 | | TRUE | II |
| 836 | cg07402669 | APCDD1L | TSS200 | Island | NA | I |
| 837 | cg07416656 | TEX1 | Body | Island | TRUE | I |
| 838 | cg07433663 | MEIS2; MEIS2; MEIS2 | 1stExon; Body; Bod | Island | NA | I |
| | | ; MEIS2; MEIS2; MEIS | y; 5′UTR; Body; Bod | | | |
| | | 2; MEIS2 | y; Body | | | |
| 839 | cg07443748 | CCT8L2; CCT8L2 | 1stExon; 5′UTR | | NA | II |
| 840 | cg07448060 | MAGI2 | TSS1500 | Island | NA | I |
| 841 | cg07459478 | VOPP1 | Body | | TRUE | I |
| 842 | cg07475654 | PNKD; TMBIM1 | Body; 5′UTR | | TRUE | II |
| 843 | cg07485357 | ADCY4 | Body | Island | NA | I |
| 844 | cg07486252 | ANK2; ANK2; ANK2 | Body; Body; Body | | TRUE | II |
| 845 | cg07525299 | ASCL2 | TSS1500 | S_Shore | NA | I |
| 846 | cg07548313 | CD1C | Body | | NA | II |
| 847 | cg07548607 | ZSWIM2 | TSS200 | Island | TRUE | I |
| 848 | cg07551545 | FBXO17; FBXO17 | 5′UTR; 1stExon | Island | TRUE | I |
| 849 | cg07560096 | ME3; ME3; ME3; ME3 | 5′UTR; 1stExon; 5′U | Island | NA | II |
| | | | TR; 5′UTR | | | |
| 850 | cg07564962 | | | Island | NA | I |
| 851 | cg07582955 | KLHL17 | Body | Island | NA | II |
| 852 | cg07587796 | ENPEP; ENPEP | 1stExon; 5′UTR | N_Shore | NA | II |
| 853 | cg07603330 | | | Island | NA | I |
| 854 | cg07620844 | RBM15B | 1stExon | N_Shore | NA | II |
| 855 | cg07627556 | | | S_Shore | TRUE | I |
| 856 | cg07629454 | CASR | 5′UTR | Island | NA | I |
| 857 | cg07641160 | FAM162B; FAM162B | 1stExon; 5′UTR | Island | NA | I |
| 858 | cg07644368 | CDO1 | TSS1500 | S_Shore | NA | II |
| 859 | cg07647825 | SLC46A3; SLC46A3 | TSS1500; TSS1500 | S_Shore | NA | II |
| 860 | cg07662771 | PSMD6 | Body | N_Shore | NA | I |
| 861 | cg07690882 | | | Island | NA | II |
| 862 | cg07697177 | C1orf86; LOC1001280 | Body; TSS200; Bod | N_Shore | NA | II |
| | | 03; C1orf86 | y | | | |
| 863 | cg07697895 | WNT2; WNT2; WNT2 | Body; 5′UTR; 1stEx | Island | NA | I |
| | | | on | | | |
| 864 | cg07702750 | TRIL | TSS200 | Island | NA | I |
| 865 | cg07758529 | ILIRAPL2 | 5′UTR | S_Shore | NA | I |
| 866 | cg07763047 | GABRB2; GABRB2 | 5′UTR; 5′UTR | Island | NA | I |
| 867 | cg07774424 | | | | NA | II |
| 868 | cg07776847 | C4orf49 | Body | Island | NA | II |
| 869 | cg07777008 | FIBIN; FIBIN | 5′UTR; 1stExon | | TRUE | I |
| 870 | cg07792478 | MIR124-2 | TSS1500 | Island | NA | I |
| 871 | cg07809589 | MEIS1 | Body | S_Shelf | NA | II |
| 872 | cg07810164 | EGFL6; EGFL6 | Body; Body | Island | TRUE | I |
| 873 | cg07810726 | EBF1 | Body | N_Shore | NA | II |
| 874 | cg07811054 | | | | NA | II |
| 875 | cg07811110 | SLC6A15; SLC6A15; S | TSS1500; TSS1500 | S_Shore | NA | I |
| | | LC6A15 | ; TSS1500 | | | |
| 876 | cg07812877 | RGE3; RGE3; CCDC15 | TSS1500; TSS1500 | Island | NA | I |
| | | 1 | ; 3′UTR | | | |
| 877 | cg07855639 | CDC42EP1 | 5′UTR | S_Shore | TRUE | I |
| 878 | cg07863022 | SEPT9; SEPT9; SEPT9 | 5′UTR; Body; TSS1 | | NA | I |
| | | | 500 | | | |
| 879 | cg07876898 | CCDC65 | TSS200 | | TRUE | II |
| 880 | cg07887243 | MAOB | 1stExon | Island | NA | I |
| 881 | cg07891531 | | | Island | TRUE | I |
| 882 | cg07903237 | | | N_Shore | NA | II |
| 883 | cg07907386 | PHOX2B | Body | Island | TRUE | I |
| 884 | cg07911353 | KIAA1549; KIAA1549 | Body; Body | | TRUE | II |
| 885 | cg07915434 | TLX1 | Body | Island | TRUE | I |
| 886 | cg07916654 | GPR123 | Body | N_Shore | NA | I |
| 887 | cg07917150 | HOXA3; HOXA3; HO | 5′UTR; TSS1500; 5′ | N_Shore | NA | II |
| | | XA3 | UTR | | | |
| 888 | cg07921314 | NRF1 | 5′UTR | S_Shore | NA | II |
| 889 | cg07964113 | RPTOR; RPTOR | Body; Body | Island | NA | I |
| 890 | cg07965640 | SPAG16; SPAG16 | Body; Body | S_Shore | NA | II |
| 891 | cg07969619 | | | Island | NA | II |
| 892 | cg07973435 | SIM1 | Body | Island | NA | I |
| 893 | cg07976064 | | | Island | NA | I |
| 894 | cg08008403 | LITX9 | Body | S_Shore | NA | II |
| 895 | cg08021564 | SPOCK2; SPOCK2 | Body; Body | Island | NA | II |
| 896 | cg08023416 | ILIRL2 | 5′UTR | Island | TRUE | II |
| 897 | cg08034379 | WDR69 | TSS200 | Island | TRUE | I |
| 898 | cg08036309 | | | N_Shore | TRUE | I |
| 899 | cg08045824 | HOXC12 | Body | S_Shore | NA | I |
| 900 | cg08048577 | TPP2 | Body | S_Shore | NA | II |
| 901 | cg08055330 | C9orf86; C9orf86; LOC | Body; Body; TSS15 | S_Shore | NA | II |
| | | 100131193 | 00 | | | |
| 902 | cg08084339 | | | | TRUE | II |
| 903 | cg08084675 | QSER1 | 5′UTR | Island | NA | II |
| 904 | cg08089364 | | | N_Shelf | NA | II |
| 905 | cg08100687 | | | Island | TRUE | II |
| 906 | cg08101036 | HOXA3; HOXA3; HO | 5′UTR; 5′UTR; TSS | N_Shore | NA | I |
| | | XA3 | 200 | | | |
| 907 | cg08128826 | LHX9; LHX9 | Body; Body | | NA | I |
| 908 | cg08140548 | SFRS11 | Body | | NA | I |
| 909 | cg08141495 | NOL4 | Body | Island | NA | II |
| 910 | cg08142344 | GSTM2; GSTM2; GST | 5′UTR; 1stExon; 1st | Island | TRUE | II |
| | | M2; GSTM2 | Exon; 5′UTR | | | |
| 911 | cg08146483 | LHX8 | Body | Island | NA | I |
| 912 | cg08151731 | FBN1 | TSS1500 | Island | NA | II |
| 913 | cg08152546 | | | Island | NA | II |
| 914 | cg08154107 | ECE2; ECE2; ECE2; EC | Body; Body; Body; | Island | TRUE | I |
| | | E2 | Body | | | |
| 915 | cg08158862 | NR5A2; NR5A2 | Body; Body | S_Shore | NA | II |
| 916 | cg08161323 | CXCL1 | TSS1500 | N_Shore | NA | II |
| 917 | cg08162372 | BMP4; BMP4 | 5′UTR; 5′UTR | Island | NA | I |
| 918 | cg08164233 | RFXAP | Body | S_Shore | NA | II |
| 919 | cg08167951 | PON3 | 1stExon | Island | NA | I |
| 920 | cg08168585 | | | N_Shelf | NA | II |
| 921 | cg08168768 | KIAA1210 | TSS1500 | Island | NA | II |
| 922 | cg08186341 | SF3B3; SNORD111B | Body; TSS1500 | | NA | I |
| 923 | cg08188069 | SDR16C6 | TSS1500 | | NA | II |
| 924 | cg08190858 | | | Island | NA | I |
| 925 | cg08196032 | | | N_Shore | NA | II |
| 926 | cg08214060 | NTM; NTM; NTM; NT | Body; Body; Body; | | NA | II |
| | | M | Body | | | |
| 927 | cg08217024 | MDGA2 | TSS1500 | Island | NA | I |
| 928 | cg08231710 | MMP23A; MMP23B | TSS1500; TSS1500 | Island | TRUE | I |
| 929 | cg08233811 | FEJ22536 | Body | S_Shore | NA | II |
| 930 | cg08238568 | RGS7 | Body | | TRUE | II |
| 931 | cg08239375 | SLC4A1AP; SUPT7L | Body; TSS1500 | S_Shore | NA | II |
| 932 | cg08263589 | | | Island | NA | I |
| 933 | cg08266417 | AOX1 | TSS200 | Island | TRUE | I |
| 934 | cg08272731 | LHX8 | Body | Island | NA | I |
| 935 | cg08320659 | HFE; HFE; HFE; HFE; H | Body; Body; Body; | | TRUE | II |
| | | FE; HFE; HFE; HFE; HF | Body; Body; Body; | | | |
| | | E | Body; Body; Body | | | |
| 936 | cg08322102 | ZFHX4; LOC10019237 | 5′UTR; Body | S_Shore | NA | I |
| | | 8 | | | | |
| 937 | cg08332074 | | | Island | NA | II |
| 938 | cg08344081 | | | Island | NA | I |
| 939 | cg08348649 | FAM70A; FAM70A; F | Body; Body; Body | N_Shore | NA | II |
| | | AM70A | | | | |
| 940 | cg08350814 | C2or28; C2orf88; C2or | TSS1500; 5′UTR; T | Island | TRUE | I |
| | | f88; C2orf88 | SS1500; 5′UTR | | | |
| 941 | cg08368617 | | | Island | TRUE | I |
| 942 | cg08368654 | | | Island | TRUE | I |
| 943 | cg08382235 | TBX1; TBX1; TBX1 | Body; Body; Body | Island | TRUE | I |
| 944 | cg08387285 | ME3; ME3; ME3 | | | | |
| | 5′UTR; TSS | Island | NA | I | II | 86383252 |
| | 200; 5′UTR | | | | | |
| 945 | cg08441803 | SKAP1; SKAP1 | Body; Body | | NA | I |
| 946 | cg08443605 | | | S _Shore | NA | I |
| 947 | cg08445802 | | | Island | NA | I |
| 948 | cg08446038 | ITGA2 | Body | | TRUE | II |
| 949 | cg08455778 | LOC645323 | Body | N_Shore | NA | I |
| 950 | cg08462988 | | | | TRUE | I |
| 951 | cg08464190 | CBLN1 | 1stExon | Island | TRUE | I |
| 952 | cg08474859 | | | Island | NA | I |
| 953 | cg08477594 | ME3; ME3; ME3; ME3 | TSS200; TSS1500; | Island | NA | II |
| | | | 1stExon; 5′UTR | | | |
| 954 | cg08479865 | DRD3; DRD3; DRD3; D | 1stExon; 5′UTR; 1st | | TRUE | II |
| | | RD3 | Exon; 5′UTR | | | |
| 955 | cg08487063 | TEX1 | Body | Island | TRUE | I |
| 956 | cg08513547 | LOCI34466 | Body | N_Shelf | NA | II |
| 957 | cg08516516 | CDO1 | TSS200 | Island | NA | I |
| 958 | cg08518715 | PARK2; PARK2; PAR | Body; Body; Body | | NA | II |
| | | K2 | | | | |
| 959 | cg08521073 | HRK | 3′UTR | N_Shelf | NA | II |
| 960 | cg08530237 | | | Island | NA | II |
| 961 | cg08546494 | | | Island | NA | II |
| 962 | cg08548396 | | | Island | NA | I |
| 963 | cg08552198 | | | Island | TRUE | II |
| 964 | cg08553437 | TMEM155; LOC10019 | TSS200; Body | Island | NA | I |
| | | 2379 | | | | |
| 965 | cg08554295 | PDE4D; PDE4D | Body; TSS1500 | S_Shore | NA | I |
| 966 | cg08560874 | HCG4 | Body | Island | NA | II |
| 967 | cg08567279 | LECT1; LECT1 | Body; Body | Island | TRUE | I |
| 968 | cg08575537 | EPO | Body | Island | TRUE | I |
| 969 | cg08585897 | KARS; TERF21P; KAR | 5′UTR; TSS1500; B | N_Shore | NA | II |
| | | S | ody | | | |
| 970 | cg08596308 | ATP6V1G2; NFKBIL1 | TSS1500; Body; Bo | | NA | II |
| | | ; NFKBIL1; NFKBIL1; | dy; Body; TSS1500; | | | |
| | | ATP6V1G2; NFKBIL1 | Body | | | |
| 971 | cg08604097 | NXPH1 | Body | N_Shore | NA | I |
| 972 | cg08605656 | SEC16A2 | Body | S_Shore | NA | I |
| 973 | cg08612384 | DHRS13 | Body | N_Shore | NA | II |
| 974 | cg08614481 | HTR1B | TSS200 | Island | NA | II |
| 975 | cg08620044 | LOC645323 | TSS1500 | Island | NA | I |
| 976 | cg08628006 | | | S_Shelf | NA | I |
| 977 | cg08673716 | | | Island | NA | II |
| 978 | cg08691567 | KRT3 | Body | Island | NA | I |
| 979 | cg08700190 | SRD5A1 | Body | S_Shore | TRUE | II |
| 980 | cg08726446 | | | Island | TRUE | II |
| 981 | cg08728495 | | | | NA | I |
| 982 | cg08736638 | | | N_Shore | NA | II |
| 983 | cg08760398 | FAM70A; FAM70A; F | Body; Body; Body | N_Shore | NA | II |
| | | AM70A | | | | |
| 984 | cg08767182 | | | Island | NA | II |
| 985 | cg08772837 | PCDHA7; PCDHAC2; | Body; TSS1500; Bo | N_Shore | NA | II |
| | | PCDHA12; PCDHA6; P | dy; Body; Body; Bod | | | |
| | | CDHA10; PCDHA4; P | y; Body; Body; Body | | | |
| | | CDHA11; PCDHA5; P | ; Body; Body; Body; | | | |
| | | CDHA6; PCDHA1; PC | Body; Body; Body; | | | |
| | | DHA2; PCDHA1; PCD | Body; Body; Body | | | |
| | | HA9; PCDHA13; PCD | | | | |
| | | HA5; PCDHAC1; PCD | | | | |
| | | HA3; PCDHA10 | | | | |
| 986 | cg08783090 | GABRE; GABRE | 1stExon; 5′UTR | Island | NA | I |
| 987 | cg08786077 | QPCT | TSS200 | Island | TRUE | I |
| 988 | cg08799778 | LMF1 | Body | N_Shelf | NA | II |
| 989 | cg08812189 | ZIC4; ZIC4; ZIC4; ZIC4 | Body; TSS200; TSS | Island | TRUE | II |
| | | ; ZIC4 | 200; Body; Body | | | |
| 990 | cg08814148 | | | Island | TRUE | II |
| 991 | cg08827307 | ADAM32 | TSS200 | Island | NA | II |
| 992 | cg08827358 | BEAN | 5′UTR | Island | NA | II |
| 993 | cg08832906 | CLEC2L | 1stExon | Island | NA | I |
| 994 | cg08844169 | TMEM39A | Body | | NA | II |
| 995 | cg08846245 | SLC39A7; RXRB; SLC | Body; TSS1500; Bo | S_Shore | NA | II |
| | | 39A7 | dy | | | |
| 996 | cg08856601 | DNAJC6 | TSS200 | N_Shore | NA | II |
| 997 | cg08876932 | PHOX2A | TSS1500 | Island | NA | I |
| 998 | cg08883223 | LHX9; LHX9 | Body; Body | N_Shore | NA | I |
| 999 | cg08884974 | | | N_Shelf | NA | II |
| 1000 | cg08886154 | PAX4 | TSS1500 | | NA | II |
| 1001 | cg08886651 | TBX1; TBX1; TBX1 | TSS1500; TSS1500 | Island | NA | I |
| | | | ; TSS1500 | | | |
| 1002 | cg08893839 | | | Island | TRUE | I |
| 1003 | cg08897924 | CC2D1B | TSS1500 | N_Shore | NA | II |
| 1004 | cg08898155 | PON3 | TSS1500 | Island | NA | I |
| 1005 | cg08924203 | MX1; MX1 | 5′UTR; 5′UTR | Island | NA | II |
| 1006 | cg08925169 | TRIM34; TRIM78P; TR | Body; TSS200; Bod | | NA | II |
| | | IM34; TRIM34; TRIM6 | y; Body; Body | | | |
| | | -TRIM34 | | | | |
| 1007 | cg08947084 | SNORD47; GAS5; SNO | TSS1500; Body; TS | N_Shelf | NA | II |
| | | RD81; SNORD80 | S1500; TSS200 | | | |
| 1008 | cg08952506 | AOX1 | TSS200 | Island | TRUE | II |
| 1009 | cg08954277 | | | S_Shore | NA | I |
| 1010 | cg08955941 | | | | NA | II |
| 1011 | cg08958015 | CCDC65; CCDC65 | 1stExon; 5′UTR | | TRUE | II |
| 1012 | cg08958294 | GRM1; GRM1 | 5′UTR; 5 ′UTR | N_Shore | NA | I |
| 1013 | cg08993172 | ALX4 | Body | Island | NA | II |
| 1014 | cg09006039 | | | | TRUE | II |
| 1015 | cg09017619 | HOXA7 | TSS200 | Island | NA | I |
| 1016 | cg09019936 | | | N_Shore | TRUE | II |
| 1017 | cg09031836 | | | N_Shelf | NA | II |
| 1018 | cg09041207 | FBX031; FBX031 | TSS1500; Body | Island | NA | II |
| 1019 | cg09065413 | PTCH2 | 3′UTR | | TRUE | II |
| 1020 | cg09066883 | PTPRN2; PTPRN2; PT | Body; Body; Body | | NA | I |
| | | PRN2 | | | | |
| 1021 | cg09072216 | SPG20; SPG20; SPG20; | 5′UTR; 5′UTR; 5′UT | N_Shore | NA | II |
| | | SPG20 | R; 5′UTR | | | |
| 1022 | cg09076431 | LHX9; LHX9 | Body; Body | N_Shore | NA | II |
| 1023 | cg09080553 | | | N_Shore | NA | I |
| 1024 | cg09092713 | | | Island | NA | II |
| 1025 | cg09094964 | | | | TRUE | I |
| 1026 | cg09100593 | SMC4; SMC4; MIR16- | Body; Body; TSS20 | S_Shelf | NA | II |
| | | 2 | 0 | | | |
| 1027 | cg09109450 | C2orf43 | 5′UTR | Island | NA | II |
| 1028 | cg09116618 | | | | TRUE | II |
| 1029 | cg09136346 | | | Island | TRUE | I |
| 1030 | cg09151979 | | | Island | NA | I |
| 1031 | cg09159022 | CSMD1 | Body | Island | NA | II |
| 1032 | cg09171882 | GLIPR1 | TSS200 | | NA | II |
| 1033 | cg09211763 | COL5A2; COL5A2 | 1stExon; 5′UTR | | NA | II |
| 1034 | cg09220563 | | | | TRUE | II |
| 1035 | cg09226388 | BMP4; BMP4; BMP4 | 5′UTR; 5′UTR; TSS | Island | NA | I |
| | | | 1500 | | | |
| 1036 | cg09234698 | | | | TRUE | II |
| 1037 | cg09236658 | | | N_Shore | NA | II |
| 1038 | cg09238801 | PNMA1; PNMA1 | 1stExon; 3′UTR | N_Shore | NA | I |
| 1039 | cg09260773 | TBX15 | 5′UTR | N_Shore | NA | II |
| 1040 | cg09275956 | | | S_Shelf | TRUE | I |
| 1041 | cg09309208 | | | | TRUE | II |
| 1042 | cg09327714 | | | Island | NA | I |
| 1043 | cg09356916 | | | | TRUE | II |
| 1044 | cg09364020 | | | S_Shore | NA | II |
| 1045 | cg09372386 | DNAH10 | Body | | NA | II |
| 1046 | cg09393254 | MCHR2; MCHR2 | TSS200; TSS200 | Island | TRUE | I |
| 1047 | cg09406238 | MTMR8 | TSS200 | Island | NA | I |
| 1048 | cg09411719 | | | | NA | I |
| 1049 | cg09418984 | MYADM | TSS200 | Island | NA | I |
| 1050 | cg09423283 | GRM1; GRM1 | 3′UTR; Body | N_Shore | NA | I |
| 1051 | cg09435415 | DPYS | TSS200 | S_Shore | NA | II |
| 1052 | cg09441363 | KCNK2; KCNK2; KCN | TSS1500; TSS1500 | Island | NA | I |
| | | K2 | ; Body | | | |
| 1053 | cg09443457 | | | S_Shore | NA | II |
| 1054 | cg09464206 | CASP8; CASP8; CASP | 5′UTR; 5′UTR; 5′UT | | NA | II |
| | | 8; CASP8; CASP8 | R; TSS1500; 5′UTR | | | |
| 1055 | cg09469566 | | | Island | NA | I |
| 1056 | cg09491897 | RPTOR; RPTOR | Body; Body | Island | NA | I |
| 1057 | cg09501509 | | | | TRUE | II |
| 1058 | cg09502069 | DEGS1; DEGS1 | Body; Body | S_Shore | NA | I |
| 1059 | cg09506473 | PSKH2 | TSS200 | Island | NA | I |
| 1060 | cg09508289 | | | | TRUE | II |
| 1061 | cg09519327 | ATG9B; NOS3 | 3′UTR; Body | Island | NA | I |
| 1062 | cg09521647 | AFF3; AFF3 | TSS1500; 5′UTR | S_Shore | TRUE | II |
| 1063 | cg09537620 | PAX6; PAX6; PAX6 | Body; Body; Body | Island | NA | I |
| 1064 | cg09544050 | BAI1 | Body | Island | TRUE | I |
| 1065 | cg09544728 | PRKAR1B; PRKAR1B | Body; Body; Body; | Island | NA | I |
| | | ; PRKAR1B; PRKAR1 | Body; Body; Body | | | |
| | | B; PRKAR1B; PRKAR | | | | |
| | | 1B | | | | |
| 1066 | cg09558195 | GRM1; GRM1 | 5′UTR; 5′UTR | Island | NA | I |
| 1067 | cg09558547 | | | | NA | II |
| 1068 | cg09577324 | OR10A2 | TSS200 | | NA | II |
| 1069 | cg09579623 | | | N_Shelf | NA | II |
| 1070 | cg09607047 | MIR365-1 | TSS1500 | | TRUE | II |
| 1071 | cg09610569 | SLITRK2; SLITRK2; S | 5′UTR; 5′UTR; 5′UT | Island | NA | I |
| | | LITRK2; SLITRK2 | R; 5′UTR | | | |
| 1072 | cg09627874 | | | N_Shore | TRUE | I |
| 1073 | cg09632273 | DDAH2 | Body | Island | NA | I |
| 1074 | cg09633973 | HIC1 | TSS1500 | Island | NA | I |
| 1075 | cg09640202 | IMP4 | Body | S_Shore | NA | II |
| 1076 | cg09645888 | ME3; ME3; ME3; ME3 | TSS200; TSS1500; | Island | NA | II |
| | | | 1stExon; 5′UTR | | | |
| 1077 | cg09658429 | | | Island | NA | II |
| 1078 | cg09670128 | KRT7; KRT7 | 5′UTR; 1stExon | Island | NA | I |
| 1079 | cg09675542 | | | | TRUE | II |
| 1080 | cg09704166 | PAX8; PAX8; PAX8; P | Body; Body; Body; | N_Shore | NA | II |
| | | AX8; PAX8; LOC4408 | Body; Body; Body | | | |
| | | 39 | | | | |
| 1081 | cg09715059 | | | | TRUE | II |
| 1082 | cg09729613 | AOX1 | TSS200 | Island | TRUE | I |
| 1083 | cg09734791 | MSC | 1stExon | Island | NA | I |
| 1084 | cg09760963 | RNF175 | Body | Island | NA | I |
| 1085 | cg09781936 | TBX1; TBX1; TBX1 | Body; Body; Body | N_Shore | NA | II |
| 1086 | cg09786278 | RGS7 | Body | | TRUE | II |
| 1087 | cg09789590 | HIF3A; HIF3A | Body; TSS1500 | Island | NA | II |
| 1088 | cg09806262 | ALOX5 | Body | S_Shore | TRUE | II |
| 1089 | cg09829319 | GCM2 | TSS200 | N_Shore | NA | II |
| 1090 | cg09839443 | QPCT | TSS200 | Island | TRUE | II |
| 1091 | cg09849405 | CMTM1; CMTM1; CM | 3′UTR; 3′UTR; TSS | Island | NA | I |
| | | TM2; CMTM1; CMTM | 1500; 3′UTR; 3′UTR | | | |
| | | 1; CMTM1; CMTM1; C | ; 3′UTR; 3′UTR; 3′U | | | |
| | | MTM1; CMTM1 | TR; 3′UTR | | | |
| 1092 | cg09866743 | ARPP-21 | 5′UTR | | NA | I |
| 1093 | cg09871637 | LMO4 | Body | N_Shore | NA | II |
| 1094 | cg09884341 | LOC645323; LOC6453 | Body; TSS1500 | N_Shore | TRUE | II |
| | | 23 | | | | |
| 1095 | cg09903452 | SEPT9; SEPT9; SEPT9 | 5′UTR; Body; TSS1 | | NA | II |
| | | | 500 | | | |
| 1096 | cg09919570 | | | Island | NA | I |
| 1097 | cg09941406 | | | S_Shore | NA | I |
| 1098 | cg09957864 | GPT | TSS1500 | N_Shore | NA | I |
| 1099 | cg09970569 | RSPO2 | Body | N_Shore | NA | II |
| 1100 | cg09970593 | OPRD1 | Body | S_Shore | NA | II |
| 1101 | cg09971314 | | | | NA | I |
| 1102 | cg09972569 | | | Island | NA | I |
| 1103 | cg09979311 | HN1L | Body | S_Shore | NA | I |
| 1104 | cg09993945 | CCAR1 | Body | S_Shore | NA | II |
| 1105 | cg10021724 | | | | NA | II |
| 1106 | cg10042106 | OTX2 | 5′UTR | Island | NA | I |
| 1107 | cg10050661 | KRT222 | TSS200 | | NA | II |
| 1108 | cg10054197 | FAM3B; FAM3B | Body; Body | Island | NA | I |
| 1109 | cg10067182 | HDAC4 | 3′UTR | Island | NA | I |
| 1110 | cg10069493 | ALOX5 | Body | Island | TRUE | I |
| 1111 | cg10106804 | GJA4 | TSS200 | Island | NA | I |
| 1112 | cg10109351 | | | N_Shore | TRUE | I |
| 1113 | cg10122477 | GLBIL3 | Body | S_Shore | NA | II |
| 1114 | cg10143323 | | | | TRUE | II |
| 1115 | cg10143811 | LMO3; LMO3 | 5′UTR; 5′UTR | | NA | I |
| 1116 | cg10160397 | ME3; ME3; ME3; ME3 | TSS200; TSS1500; | Island | NA | II |
| | | | 1stExon; 5′UTR | | | |
| 1117 | cg10174333 | | | | NA | II |
| 1118 | cg10182317 | CLVS2 | TSS200 | Island | NA | I |
| 1119 | cg10223827 | NRN1; NRN1 | 5′UTR; 1stExon | Island | TRUE | I |
| 1120 | cg10233654 | DUSP6; DUSP6 | Body; Body | N_Shore | NA | I |
| 1121 | cg10244666 | WT1; WT1; WT1; WT1 | Body; Body; Body; | | NA | I |
| | | | Body | | | |
| 1122 | cg10249375 | | | Island | TRUE | I |
| 1123 | cg10259748 | C6orf186 | Body | Island | TRUE | I |
| 1124 | cg10263682 | SOX2OT | Body | Island | TRUE | I |
| 1125 | cg10264687 | | | N_Shore | TRUE | II |
| 1126 | cg10271061 | ATP6V1G2; NFKBIL1 | TSS1500; Body; Bo | | NA | I |
| | | ; NFKBIL1; NFKBIL1; | dy; Body; TSS1500; | | | |
| | | ATP6V1G2; NFKBIL1 | Body | | | |
| 1127 | cg10280007 | FGFR2; FGFR2; FGFR | Body; Body; Body; | | TRUE | II |
| | | 2; FGFR2; FGFR2; FGF | Body; Body; Body; | | | |
| | | R2; FGFR2; FGFR2; FG | Body; Body; Body | | | |
| | | FR2 | | | | |
| 1128 | cg10289744 | CTIL1 | 5′UTR | | TRUE | II |
| 1129 | cg10303487 | DPYS | 1stExon | Island | NA | I |
| 1130 | cg10308673 | | | | TRUE | I |
| 1131 | cg10314133 | C1orf86; LOC1001280 | Body; TSS200; Bod | N_Shore | NA | II |
| | | 03; C1orf86 | y | | | |
| 1132 | cg10320410 | | | | NA | I |
| 1133 | cg10321196 | LOC100303728; SLC2 | Body; TSS1500 | N_Shore | NA | II |
| | | 5A5 | | | | |
| 1134 | cg10327400 | ERFN5 | TSS1500 | N_Shore | NA | II |
| 1135 | cg10330056 | | | | TRUE | II |
| 1136 | cg10356187 | | | Island | TRUE | II |
| 1137 | cg10375078 | | | Island | TRUE | I |
| 1138 | cg10375890 | | | Island | NA | I |
| 1139 | cg10377903 | | | Island | NA | I |
| 1140 | cg10383428 | NCAPD2 | 5′UTR | S_Shore | NA | II |
| 1141 | cg10393598 | | | Island | NA | I |
| 1142 | cg10397527 | HLA-DOA | Body | S_Shore | NA | I |
| 1143 | cg10401362 | DNAJB6 | Body | | NA | II |
| 1144 | cg10417567 | NKX6-1 | Body | Island | TRUE | I |
| 1145 | cg10435816 | PCDHGA4; PCDHGA | Body; Body; Body; | Island | NA | I |
| | | 9; PCDHGA1 ; PCDHG | Body; TSS200; Bod | | | |
| | | B1; PCDHGB7; PCDH | y; Body; TSS200; Bo | | | |
| | | GB6; PCDHGB3; PCD | dy; Body; Body; Bod | | | |
| | | HGB7; PCDHGA6; PC | y; Body; Body; Body | | | |
| | | DHGA5; PCDHGA10; | ; Body; Body; Body | | | |
| | | PCDHGA5; PCDHGB4 | | | | |
| | | ; PCDHGA3; PCDHGA | | | | |
| | | 2; PCDHGB2; PCDHG | | | | |
| | | A7; PCDHGB5 | | | | |
| 1146 | cg10440696 | OR52N1 | 1stExon | | NA | II |
| 1147 | cg10443049 | NXPH1 | Body | N_Shore | NA | II |
| 1148 | cg10446179 | GJA1 | 5′UTR | | NA | II |
| 1149 | cg10453019 | GALNTL2; GALNTL2 | 5′UTR; 1stExon | | TRUE | II |
| 1150 | cg10483275 | CAMK2A; CAMK2A; | 5′UTR; 1stExon; 5′U | | NA | II |
| | | CAMK2A; CAMK2A | TR; 1stExon | | | |
| 1151 | cg10499172 | DCPS | TSS1500 | N_Shore | NA | II |
| 1152 | cg10504392 | | | | TRUE | I |
| 1153 | cg10512745 | DMRTA2 | Body | Island | NA | I |
| 1154 | cg10513702 | NR5A2; NR5A2 | Body; Body | N_Shore | NA | I |
| 1155 | cg10519368 | PCDHA6; PCDHA2; P | Body; Body; Body; | N_Shore | NA | II |
| | | CDHA1; PCDHA9; PC | Body; Body; Body; | | | |
| | | DHA7; PCDHA1; PCD | Body; TSS200; Bod | | | |
| | | HA6; PCDHA10; PCD | y; Body; Body; TSS2 | | | |
| | | HA5; PCDHA3; PCDH | 00; TSS200; Body | | | |
| | | A4; PCDHA10; PCDH | | | | |
| | | A10; PCDHA8 | | | | |
| 1156 | cg10552126 | DPP6; DPP6; DPP6 | TSS1500; Body; Bo | Island | TRUE | I |
| | | | dy | | | |
| 1157 | cg10558887 | SPG20; SPG20; SPG20; | 5′UTR; 5′UTR; 5′UT | N_Shore | NA | I |
| | | SPG20 | R; 5′UTR | | | |
| 1158 | cg10578938 | CYFIP2; CYFIP2; CYF | 5′UTR; TSS1500; 5′ | S_Shore | TRUE | II |
| | | IP2 | UTR | | | |
| 1159 | cg10583997 | BIRC6 | Body | S_Shore | NA | II |
| 1160 | cg10593416 | | | Island | NA | II |
| 1161 | cg10600889 | FBXO47 | TSS200 | | NA | II |
| 1162 | cg10603290 | TRIO | Body | | TRUE | II |
| 1163 | cg10606490 | C11orf88; C11orf88 | TSS200; TSS200 | Island | NA | I |
| 1164 | cg10613701 | | | N_Shore | NA | II |
| 1165 | cg10633601 | TAL1 | TSS1500 | Island | TRUE | I |
| 1166 | cg10658542 | SYT10; SYT10 | 1stExon; 5′UTR | Island | NA | I |
| 1167 | cg10667338 | LIN7B | TSS1500 | N_Shore | NA | I |
| 1168 | cg10671180 | SNORD115-39 | TSS200 | | NA | II |
| 1169 | cg10687131 | GDF7 | Body | Island | NA | II |
| 1170 | cg10696085 | | | | TRUE | I |
| 1171 | cg10699857 | | | Island | TRUE | II |
| 1172 | cg10703826 | TBX15; TBX15 | 1stExon; 5′UTR | Island | TRUE | I |
| 1173 | cg10706039 | | | Island | NA | I |
| 1174 | cg10719363 | | | N_Shelf | NA | I |
| 1175 | cg10745499 | SIM1 | Body | Island | NA | II |
| 1176 | cg10747118 | | | S_Shore | NA | I |
| 1177 | cg10752158 | | | N_Shelf | NA | II |
| 1178 | cg10758466 | MPP2 | Body | N_Shelf | NA | II |
| 1179 | cg10776919 | DMRTA2 | TSS1500 | Island | NA | I |
| 1180 | cg10784570 | | | Island | NA | II |
| 1181 | cg10785622 | | | N_Shore | NA | II |
| 1182 | cg10786329 | | | S_Shore | NA | II |
| 1183 | cg10799055 | | | | NA | I |
| 1184 | cg10801213 | SLC7A3; SLC7A3 | 5′UTR; 5′UTR | Island | NA | II |
| 1185 | cg10812708 | | | N_Shore | TRUE | I |
| 1186 | cg10826197 | | | S_Shore | TRUE | I |
| 1187 | cg10858215 | | | Island | NA | I |
| 1188 | cg10862468 | CYP2E1 | Body | Island | NA | II |
| 1189 | cg10864074 | EPO | TSS1500 | Island | TRUE | I |
| 1190 | cg10876767 | SORCS2 | Body | | NA | I |
| 1191 | cg10888031 | ACTRT2 | 1stExon | Island | NA | I |
| 1192 | cg10893095 | | | | NA | I |
| 1193 | cg10911913 | SLFN11; SLFN11; SLF | TSS200; TSS200; T | N_Shore | TRUE | II |
| | | N11; SLFN11; SLFN11 | SS200; TSS200; TS | | | |
| | | | S200 | | | |
| 1194 | cg10923838 | CIT | Body | S_Shore | TRUE | II |
| 1195 | cg10948359 | ME1; ME1 | 5′UTR; 1stExon | Island | NA | I |
| 1196 | cg10950028 | GSTM1; GSTM1 | Body; Body | S_Shore | NA | I |
| 1197 | cg10959711 | S100A6 | TSS1500 | S_Shore | NA | I |
| 1198 | cg11029716 | | | | TRUE | II |
| 1199 | cg11035122 | MIR758; MIR329-2; | TSS1500; TSS1500 | | NA | II |
| 1200 | cg11044163 | MIR329-1; MIR323 | ; TSS1500; TSS200 | | TRUE | II |
| | | CPVL; CPVL | TSS200; TSS200 | | | |
| 1201 | cg11062466 | | | N_Shore | NA | I |
| 1202 | cg11062848 | NOL7 | Body | S_Shore | NA | II |
| 1203 | cg11071926 | NHLH2; NHLH2 | 3′UTR; 3′UTR | N_Shore | NA | II |
| 1204 | cg11120913 | C3orf14 | TSS1500 | N_Shore | NA | II |
| 1205 | cg11143193 | | | N_Shelf | NA | II |
| 1206 | cg11144753 | | | Island | TRUE | I |
| 1207 | cg11145160 | CLDN1 | TSS1500 | Island | NA | I |
| 1208 | cg11151395 | MPO | Body | Island | NA | I |
| 1209 | cg11166197 | GABRA3; MIR105-1 | 5′UTR; TSS1500 | | NA | II |
| 1210 | cg11171221 | | | S_Shore | NA | I |
| 1211 | cg11194545 | NRK; NRK | 5′UTR; 1stExon | Island | NA | I |
| 1212 | cg11195360 | OR8B8 | TSS1500 | | NA | II |
| 1213 | cg11207307 | SLC2A14 | TSS200 | Island | NA | I |
| 1214 | cg11220565 | | | N_Shore | TRUE | I |
| 1215 | cg11229513 | PRDM14 | TSS1500 | Island | NA | I |
| 1216 | cg11231249 | CTNNA2; LRRTM1; C | Body; TSS200; Bod | Island | NA | II |
| | | TNNA2 | y | | | |
| 1217 | cg11237792 | TTC30A | TSS1500 | S_Shore | NA | II |
| 1218 | cg11241627 | FERD3L | TSS200 | S_Shore | TRUE | II |
| 1219 | cg11258089 | PDE4D; PDE4D | Body; TSS200 | Island | TRUE | I |
| 1220 | cg11268834 | MRPL44 | Body | S_Shore | NA | II |
| 1221 | cg11269103 | ZNF215 | Body | | NA | II |
| 1222 | cg11274314 | | | Island | NA | I |
| 1223 | cg11284797 | | | Island | NA | I |
| 1224 | cg11309496 | SLFN11; SLFN11; SLF | TSS1500; TSS1500 | Island | TRUE | I |
| | | N11; SLFN11; SLFN11 | ; TSS1500; TSS150 | | | |
| | | | 0; TSS1500 | | | |
| 1225 | cg11312408 | PCDH17 | 1stExon | Island | NA | II |
| 1226 | cg11323198 | CDH8 | 5′UTR | Island | NA | II |
| 1227 | cg11337289 | DZIP1; DZIP1 | Body; Body | Island | TRUE | II |
| 1228 | cg11337915 | HMGN1 | Body | N_Shore | NA | II |
| 1229 | cg11347582 | | | Island | NA | I |
| 1230 | cg11367159 | | | | TRUE | II |
| 1231 | cg11382963 | MSC | 3′UTR | Island | NA | I |
| 1232 | cg11386025 | KCNQ4; KCNQ4 | Body; Body | Island | TRUE | I |
| 1233 | cg11389889 | NOP56; NOP56; SNOR | Body; Body; Body; T | S_Shore | NA | II |
| | | D110; SNORA51 | SS1500 | | | |
| 1234 | cg11401794 | NKD2 | Body | | NA | I |
| 1235 | cg11409022 | ST3GAL3; ST3GAL3; | Body; Body; Body; | | TRUE | II |
| | | ST3GAL3; ST3GAL3; | Body; Body; Body; | | | |
| | | ST3GAL3; ST3GAL3; | Body; Body; Body; | | | |
| | | ST3GAL3; ST3GAL3; | Body | | | |
| | | ST3GAL3; ST3GAL3 | | | | |
| 1236 | cg11410682 | PNMAL2 | TSS200 | Island | NA | I |
| 1237 | cg11412129 | | | S_Shore | NA | I |
| 1238 | cg11412853 | | | N_Shore | TRUE | II |
| 1239 | cg11416384 | KIAA1239 | 1stExon | Island | NA | I |
| 1240 | cg11431957 | VILL | Body | Island | NA | II |
| 1241 | cg11432962 | | | Island | TRUE | II |
| 1242 | cg11433319 | CHODL | Body | Island | NA | I |
| 1243 | cg11435506 | PON3 | TSS1500 | Island | NA | I |
| 1244 | cg11439596 | | | Island | NA | I |
| 1245 | cg11452329 | | | | NA | I |
| 1246 | cg11458217 | LOC644538; LOC6445 | 1stExon; 5′UTR | Island | TRUE | I |
| | | 38 | | | | |
| 1247 | cg11459603 | HIST1H2BI | 1stExon | Island | NA | II |
| 1248 | cg11460714 | | | | NA | II |
| 1249 | cg11471772 | SIM1 | TSS200 | N_Shore | TRUE | I |
| 1250 | cg11485152 | TRNT1 | Body | S_Shelf | NA | II |
| 1251 | cg11485595 | NPR3 | Body | Island | NA | II |
| 1252 | cg11497372 | | | | NA | I |
| 1253 | cg11503104 | C1orf86; LOC1001280 | Body; TSS200; Bod | N_Shore | NA | II |
| | | 03; C1orf86 | y | | | |
| 1254 | cg11504718 | ATP11A; ATP11A | 3′UTR; 3′UTR | S_Shore | NA | II |
| 1255 | cg11521404 | GJA3 | TSS1500 | Island | NA | I |
| 1256 | cg11521965 | PTN | TSS200 | | NA | II |
| 1257 | cg11525280 | C2orf39 | TSS200 | Island | TRUE | I |
| 1258 | cg11550547 | KCTD14 | TSS200 | | NA | II |
| 1259 | cg11564981 | NRN1 | TSS200 | Island | TRUE | I |
| 1260 | cg11567288 | | | | NA | II |
| 1261 | cg11574975 | ADORA3 | Body | Island | TRUE | I |
| 1262 | cg11580212 | | | | TRUE | II |
| 1263 | cg11580948 | FGF5; FGF5 | Body; Body | S_Shore | NA | II |
| 1264 | cg11593123 | PCDH11X | 5′UTR | Island | NA | II |
| 1265 | cg11595135 | | | Island | NA | II |
| 1266 | cg11601252 | LBXCOR1 | Body | Island | NA | I |
| 1267 | cg11605903 | | | S_Shelf | NA | II |
| 1268 | cg11616651 | AMH | Body | Island | NA | I |
| 1269 | cg11640591 | PTH1R | Body | Island | NA | II |
| 1270 | cg11641791 | KRT222 | TSS200 | | NA | I |
| 1271 | cg11642106 | | | S_Shore | TRUE | I |
| 1272 | cg11645465 | | | | NA | II |
| 1273 | cg11648594 | MMP23A; MMP23B | TSS1500; TSS1500 | Island | TRUE | I |
| 1274 | cg11671688 | GPR6 | 1stExon | Island | NA | I |
| 1275 | cg11678027 | C13orf38; C13orf38; C1 | TSS200; TSS200; T | Island | NA | I |
| | | 3orf38; C13orf38; C13o | SS200; TSS200; TS | | | |
| | | rf38; C13orf38 | S200; TSS200 | | | |
| 1276 | cg11680055 | GSTM1; GSTM1 | TSS200; TSS200 | Island | NA | I |
| 1277 | cg11684647 | LRRK2 | TSS1500 | Island | NA | II |
| 1278 | cg11687036 | | | Island | TRUE | II |
| 1279 | cg11690724 | EBF1 | Body | N_Shelf | NA | II |
| 1280 | cg11690979 | C1orf230 | Body | S_Shore | NA | II |
| 1281 | cg11708358 | LRP11 | Body | N_Shore | NA | II |
| 1282 | cg11712990 | LOC645323; LOC6453 | Body; Body; Body | N_Shore | NA | I |
| | | 23; LOC645323 | | | | |
| 1283 | cg11720950 | | | | TRUE | II |
| 1284 | cg11731890 | HIST1H3G; HISTIH2 | TSS1500; TSS1500 | N_Shore | NA | II |
| | | BI | | | | |
| 1285 | cg11735997 | KCNA4 | TSS1500 | Island | NA | II |
| 1286 | cg11737164 | VGLL3 | Body | N_Shore | NA | II |
| 1287 | cg11739675 | CHST11 | Body | Island | NA | I |
| 1288 | cg11748187 | TCF7L2; TCF7L2; TCF | Body; Body; Body; | S_Shore | NA | II |
| | | 7L2; TCF7L2; TCF7L2; | Body; Body; Body | | | |
| | | TCF7L2 | | | | |
| 1289 | cg11756095 | | | Island | TRUE | I |
| 1290 | cg11791751 | PDPN; PDPN | TSS200; TSS200 | Island | NA | II |
| 1291 | cg11805669 | | | S_Shore | NA | I |
| 1292 | cg11811513 | ZNF804A | TSS1500 | N_Shore | NA | II |
| 1293 | cg11832210 | SLC16A12 | TSS200 | Island | TRUE | I |
| 1294 | cg11833001 | | | Island | NA | II |
| 1295 | cg11840125 | EFS; EFS | TSS200; TSS200 | Island | NA | I |
| 1296 | cg11856078 | | | S_Shore | NA | I |
| 1297 | cg11856897 | | | Island | TRUE | I |
| 1298 | cg11857704 | | | Island | NA | I |
| 1299 | cg11859529 | SLC7A3; SLC7A3; SL | 1stExon; 5′UTR; 1st | Island | NA | I |
| | | C7A3; SLC7A3 | Exon; 5′UTR | | | |
| 1300 | cg11861562 | TAGEN; TAGEN | TSS1500; TSS1500 | | NA | I |
| 1301 | cg11874123 | PTN; PTN | 5′UTR; 1stExon | | NA | II |
| 1302 | cg11889692 | | | Island | TRUE | I |
| 1303 | cg11891925 | | | | NA | II |
| 1304 | cg11898009 | | | Island | TRUE | I |
| 1305 | cg11898486 | LYPD5; LYPD5 | 5′UTR; 1stExon | Island | NA | I |
| 1306 | cg11900861 | FEJ22536 | TSS200 | Island | NA | II |
| 1307 | cg11901043 | | | Island | NA | I |
| 1308 | cg11935153 | | | Island | TRUE | II |
| 1309 | cg11947981 | ITGA4 | Body | Island | NA | II |
| 1310 | cg11953383 | ME3; ME3; ME3 | 5′UTR; TSS200; 5′U | Island | NA | I |
| | | | TR | | | |
| 1311 | cg11973479 | | | Island | NA | II |
| 1312 | cg11980129 | ROBO1 | 5′UTR | Island | NA | II |
| 1313 | cg11982736 | CRIP3 | TSS200 | Island | TRUE | I |
| 1314 | cg12005098 | SLC16A12 | TSS200 | Island | TRUE | I |
| 1315 | cg12011198 | HDAC4 | 3′UTR | Island | NA | I |
| 1316 | cg12014547 | NELL1; NELL1 | Body; Body | S_Shore | NA | I |
| 1317 | cg12016746 | LRP5 | Body | S_Shelf | NA | II |
| 1318 | cg12019614 | DOCK6 | Body | Island | TRUE | I |
| 1319 | cg12029804 | KRT222; KRT222 | 5′UTR; 1stExon | | NA | II |
| 1320 | cg12030550 | ME3; ME3; ME3 | TSS1500; TSS200; | S_Shore | NA | II |
| | | | TSS1500 | | | |
| 1321 | cg12034871 | ATXN3; ATXN3; ATX | Body; 3′UTR; Body; | | TRUE | II |
| | | N3; ATXN3; ATXN3; A | Body; Body; 3′UTR; | | | |
| | | TXN3; ATXN3; ATXN | Body; Body; 3′UTR; | | | |
| | | 3; ATXN3; ATXN3; AT | Body; 3′UTR; 3′UT | | | |
| | | XN3; ATXN3; ATXN3; | R; Body; Body; 3′UT | | | |
| | | ATXN3; ATXN3; ATX | R; Body; Body; Bod | | | |
| | | N3; ATXN3; ATXN3; A | y; 3′UTR; Body; 3′U | | | |
| | | TXN3; ATXN3; ATXN | TR; 3′UTR; Body; B | | | |
| | | 3; ATXN3; ATXN3; AT | ody; Body; 3′UTR; 3′ | | | |
| | | XN3; ATXN3; ATXN3; | UTR; 3′UTR; Body; | | | |
| | | ATXN3; ATXN3; ATX | Body; Body; 3′UTR | | | |
| | | N3; ATXN3; ATXN3; A | | | | |
| | | TXN3 | | | | |
| 1322 | cg12052765 | SLC18A3; CHAT | TSS1500; TSS200 | N_Shore | NA | II |
| 1323 | cg12090430 | ADARB2 | Body | | NA | II |
| 1324 | cg12093519 | | | N_Shore | TRUE | II |
| 1325 | cg12103152 | HTR1B | TSS200 | Island | NA | II |
| 1326 | cg12106728 | | | Island | NA | I |
| 1298 | cg11857704 | | | Island | NA | I |
| 1299 | cg11859529 | SLC7A3; SLC7A3; SL | 1stExon; 5′UTR; 1st | Island | NA | I |
| | | C7A3; SLC7A3 | Exon; 5′UTR | | | |
| 1300 | cg11861562 | TAGEN; TAGEN | TSS1500; TSS1500 | | NA | I |
| 1301 | cg11874123 | PTN; PTN | 5′UTR; 1stExon | | NA | II |
| 1302 | cg11889692 | | | Island | TRUE | 1 |
| 1303 | cg11891925 | | | | NA | II |
| 1304 | cg11898009 | | | Island | TRUE | I |
| 1305 | cg11898486 | LYPD5; LYPD5 | 5′UTR; 1stExon | Island | NA | I |
| 1306 | cg11900861 | FLJ22536 | TSS200 | Island | NA | II |
| 1307 | cg11901043 | | | Island | NA | I |
| 1308 | cg11935153 | | | Island | TRUE | II |
| 1309 | cg11947981 | ITGA4 | Body | Island | NA | II |
| 1310 | cg11953383 | ME3; ME3; ME3 | 5′UTR; TSS200; 5′U | Island | NA | I |
| | | | TR | | | |
| 1311 | cg11973479 | | | Island | NA | II |
| 1312 | cg11980129 | ROBO1 | 5′UTR | Island | NA | II |
| 1313 | cg11982736 | CRIP3 | TSS200 | Island | TRUE | I |
| 1314 | cg12005098 | SLC16A12 | TSS200 | Island | TRUE | I |
| 1315 | cg12011198 | HDAC4 | 3′UTR | Island | NA | I |
| 1316 | cg12014547 | NELL1; NELL1 | Body; Body | S_Shore | NA | I |
| 1317 | cg12016746 | LRP5 | Body | S_Shelf | NA | II |
| 1318 | cg12019614 | DOCK6 | Body | Island | TRUE | I |
| 1319 | cg12029804 | KRT222; KRT222 | 5′UTR; 1stExon | | NA | II |
| 1320 | cg12030550 | ME3; ME3; ME3 | TSS1500; TSS200; | S_Shore | NA | II |
| | | | TSS1500 | | | |
| 1321 | cg12034871 | ATXN3; ATXN3; ATX | Body; 3′UTR; Body; | | TRUE | II |
| | | N3; ATXN3; ATXN3; A | Body; Body; 3′UTR; | | | |
| | | TXN3; ATXN3; ATXN | Body; Body; 3′UTR; | | | |
| | | 3; ATXN3; ATXN3; AT | Body; 3′UTR; 3′UT | | | |
| | | XN3; ATXN3; ATXN3; | R; Body; Body; 3′UT | | | |
| | | ATXN3; ATXN3; ATX | R; Body; Body; Bod | | | |
| | | N3; ATXN3; ATXN3; A | y; 3′UTR; Body; 3′U | | | |
| | | TXN3; ATXN3; ATXN | TR; 3′UTR; Body; B | | | |
| | | 3; ATXN3; ATXN3; AT | ody; Body; 3′UTR; 3′ | | | |
| | | XN3; ATXN3; ATXN3; | UTR; 3′UTR; Body; | | | |
| | | ATXN3; ATXN3; ATX | Body; Body; 3′UTR | | | |
| | | N3; ATXN3; ATXN3; A | | | | |
| | | TXN3 | | | | |
| 1322 | cg12052765 | SEC18A3; CHAT | TSS1500; TSS200 | N_Shore | NA | II |
| 1323 | cg12090430 | ADARB2 | Body | | NA | II |
| 1324 | cg12093519 | | | N_Shore | TRUE | II |
| 1325 | cg12103152 | HTR1B | TSS200 | Island | NA | II |
| 1326 | cg12106728 | | | Island | NA | 1 |
| 1298 | cg11857704 | | | Island | NA | I |
| 1299 | cg11859529 | SLC7A3; SLC7A3; SL | 1stExon; 5′UTR; 1st | Island | NA | I |
| | | C7A3; SLC7A3 | Exon; 5′UTR | | | |
| 1300 | cg11861562 | TAGEN; TAGEN | TSS1500; TSS1500 | | NA | I |
| 1301 | cg11874123 | PTN; PTN | 5′UTR; 1stExon | | NA | II |
| 1302 | cg11889692 | | | Island | TRUE | I |
| 1303 | cg11891925 | | | | NA | II |
| 1304 | cg11898009 | | | Island | TRUE | I |
| 1305 | cg11898486 | LYPD5; LYPD5 | 5′UTR; 1stExon | Island | NA | I |
| 1306 | cg11900861 | FEJ22536 | TSS200 | Island | NA | II |
| 1307 | cg11901043 | | | Island | NA | I |
| 1308 | cg11935153 | | | Island | TRUE | II |
| 1309 | cg11947981 | ITGA4 | Body | Island | NA | II |
| 1310 | cg11953383 | ME3; ME3; ME3 | 5′UTR; TSS200; 5′U | Island | NA | I |
| | | | TR | | | |
| 1311 | cg11973479 | | | Island | NA | II |
| 1312 | cg11980129 | ROBO1 | 5′UTR | Island | NA | II |
| 1313 | cg11982736 | CRIP3 | TSS200 | Island | TRUE | I |
| 1314 | cg12005098 | SLC16A12 | TSS200 | Island | TRUE | I |
| 1315 | cg12011198 | HDAC4 | 3′UTR | Island | NA | I |
| 1316 | cg12014547 | NELL1; NELL1 | Body; Body | S_Shore | NA | I |
| 1317 | cg12016746 | LRP5 | Body | S_Shelf | NA | II |
| 1318 | cg12019614 | DOCK6 | Body | Island | TRUE | I |
| 1319 | cg12029804 | KRT222; KRT222 | 5′UTR; 1stExon | | NA | II |
| 1320 | cg12030550 | ME3; ME3; ME3 | TSS1500; TSS200; | S_Shore | NA | II |
| | | | TSS1500 | | | |
| 1321 | cg12034871 | ATXN3; ATXN3; ATX | Body; 3′UTR; Body; | | TRUE | II |
| | | N3; ATXN3; ATXN3; A | Body; Body; 3′UTR; | | | |
| | | TXN3; ATXN3; ATXN | Body; Body; 3′UTR; | | | |
| | | 3; ATXN3; ATXN3; AT | Body; 3′UTR; 3′UT | | | |
| | | XN3; ATXN3; ATXN3; | R; Body; Body; 3′UT | | | |
| | | ATXN3; ATXN3; ATX | R; Body; Body; Bod | | | |
| | | N3; ATXN3; ATXN3; A | y; 3′UTR; Body; 3′U | | | |
| | | TXN3; ATXN3; ATXN | TR; 3′UTR; Body; B | | | |
| | | 3; ATXN3; ATXN3; AT | ody; Body; 3′UTR; 3′ | | | |
| | | XN3; ATXN3; ATXN3; | UTR; 3′UTR; Body; | | | |
| | | ATXN3; ATXN3; ATX | Body; Body; 3′UTR | | | |
| | | N3; ATXN3; ATXN3; A | | | | |
| | | TXN3 | | | | |
| 1322 | cg12052765 | SEC18A3; CHAT | TSS1500; TSS200 | N_Shore | NA | II |
| 1323 | cg12090430 | ADARB2 | Body | | NA | II |
| 1324 | cg12093519 | | | N_Shore | TRUE | II |
| 1325 | cg12103152 | HTR1B | TSS200 | Island | NA | II |
| 1326 | cg12106728 | | | Island | NA | I |
| 1298 | cg11857704 | | | Island | NA | I |
| 1299 | cg11859529 | SLC7A3; SLC7A3; SL | 1stExon; 5′UTR; 1st | Island | NA | I |
| | | C7A3; SLC7A3 | Exon; 5′UTR | | | |
| 1300 | cg11861562 | TAGEN; TAGEN | TSS1500; TSS1500 | | NA | I |
| 1301 | cg11874123 | PTN; PTN | 5′UTR; 1stExon | | NA | II |
| 1302 | cg11889692 | | | Island | TRUE | I |
| 1303 | cg11891925 | | | | NA | II |
| 1304 | cg11898009 | | | Island | TRUE | I |
| 1305 | cg11898486 | LYPD5; LYPD5 | 5′UTR; 1stExon | Island | NA | I |
| 1306 | cg11900861 | FLJ22536 | TSS200 | Island | NA | II |
| 1307 | cg11901043 | | | Island | NA | I |
| 1308 | cg11935153 | | | Island | TRUE | II |
| 1309 | cg11947981 | ITGA4 | Body | Island | NA | II |
| 1310 | cg11953383 | ME3; ME3; ME3 | 5′UTR; TSS200; 5′U | Island | NA | I |
| | | | TR | | | |
| 1311 | cg11973479 | | | Island | NA | II |
| 1312 | cg11980129 | ROBO1 | 5′UTR | Island | NA | II |
| 1313 | cg11982736 | CRIP3 | TSS200 | Island | TRUE | I |
| 1314 | cg12005098 | SLC16A12 | TSS200 | Island | TRUE | I |
| 1315 | cg12011198 | HDAC4 | 3′UTR | Island | NA | I |
| 1316 | cg12014547 | NELL1; NELL1 | Body; Body | S_Shore | NA | I |
| 1317 | cg12016746 | LRP5 | Body | S_Shelf | NA | II |
| 1318 | cg12019614 | DOCK6 | Body | Island | TRUE | I |
| 1319 | cg12029804 | KRT222; KRT222 | 5′UTR; 1stExon | | NA | II |
| 1320 | cg12030550 | ME3; ME3; ME3 | TSS1500; TSS200; | | | |
| TSS1 | S_Shore | NA | II | II | 86383713 | GGGGATTT |
| 500 | | | | | | GCGTTTGG |
| | | | | | | GGGTCAGC |
| | | | | | | ATATCCTC |
| | | | | | | GCACTCTC |
| | | | | | | CCAACATT |
| | | | | | | CG |
| 1321 | cg12034871 | ATXN3; ATXN3; ATX | Body; 3′UTR; Body; | | TRUE | II |
| | | N3; ATXN3; ATXN3; A | Body; Body; 3′UTR; | | | |
| | | TXN3; ATXN3; ATXN | Body; Body; 3′UTR; | | | |
| | | 3; ATXN3; ATXN3; AT | Body; 3′UTR; 3′UT | | | |
| | | XN3; ATXN3; ATXN3; | R; Body; Body; 3′UT | | | |
| | | ATXN3; ATXN3; ATX | R; Body; Body; Bod | | | |
| | | N3; ATXN3; ATXN3; A | y; 3′UTR; Body; 3′U | | | |
| | | TXN3; ATXN3; ATXN | TR; 3′UTR; Body; B | | | |
| | | 3; ATXN3; ATXN3; AT | ody; Body; 3′UTR; 3′ | | | |
| | | XN3; ATXN3; ATXN3; | UTR; 3′UTR; Body; | | | |
| | | ATXN3; ATXN3; ATX | Body; Body; 3′UTR | | | |
| | | N3; ATXN3; ATXN3; A | | | | |
| | | TXN3 | | | | |
| 1322 | cg12052765 | SEC18A3; CHAT | TSS1500; TSS200 | N_Shore | NA | II |
| 1323 | cg12090430 | ADARB2 | Body | | NA | II |
| 1324 | cg12093519 | | | N_Shore | TRUE | II |
| 1325 | cg12103152 | HTR1B | TSS200 | Island | NA | II |
| 1326 | cg12106728 | | | Island | NA | I |
| 1298 | cg11857704 | | | Island | NA | I |
| 1299 | cg11859529 | SLC7A3; SLC7A3; SL | 1stExon; 5′UTR; 1st | Island | NA | I |
| | | C7A3; SLC7A3 | Exon; 5′UTR | | | |
| 1300 | cg11861562 | TAGEN; TAGEN | TSS1500; TSS1500 | | NA | I |
| 1301 | cg11874123 | PTN; PTN | 5′UTR; 1stExon | | NA | II |
| 1302 | cg11889692 | | | Island | TRUE | I |
| 1303 | cg11891925 | | | | NA | II |
| 1304 | cg11898009 | | | Island | TRUE | I |
| 1305 | cg11898486 | LYPD5; LYPD5 | 5′UTR; 1stExon | Island | NA | I |
| 1306 | cg11900861 | FLJ22536 | TSS200 | Island | NA | II |
| 1307 | cg11901043 | | | Island | NA | I |
| 1308 | cg11935153 | | | Island | TRUE | II |
| 1309 | cg11947981 | ITGA4 | Body | Island | NA | II |
| 1310 | cg11953383 | ME3; ME3; ME3 | 5′UTR; TSS200; 5′U | Island | NA | I |
| | | | TR | | | |
| 1311 | cg11973479 | | | Island | NA | II |
| 1312 | cg11980129 | ROBO1 | 5′UTR | Island | NA | II |
| 1313 | cg11982736 | CRIP3 | TSS200 | Island | TRUE | I |
| 1314 | cg12005098 | SLC16A12 | TSS200 | Island | TRUE | I |
| 1315 | cg12011198 | HDAC4 | 3′UTR | Island | NA | I |
| 1316 | cg12014547 | NELL1; NELL1 | Body; Body | S_Shore | NA | I |
| 1317 | cg12016746 | LRP5 | Body | S_Shelf | NA | II |
| 1318 | cg12019614 | DOCK6 | Body | Island | TRUE | I |
| 1319 | cg12029804 | KRT222; KRT222 | 5′UTR; 1stExon | | NA | II |
| 1320 | cg12030550 | ME3; ME3; ME3 | TSS1500; TSS200; | S_Shore | NA | II |
| | | | TSS1500 | | | |
| 1321 | cg12034871 | ATXN3; ATXN3; ATX | Body; 3′UTR; Body; | | TRUE | II |
| | | N3; ATXN3; ATXN3; A | Body; Body; 3′UTR; | | | |
| | | TXN3; ATXN3; ATXN | Body; Body; 3′UTR; | | | |
| | | 3; ATXN3; ATXN3; AT | Body; 3′UTR; 3′UT | | | |
| | | XN3; ATXN3; ATXN3; | R; Body; Body; 3′UT | | | |
| | | ATXN3; ATXN3; ATX | R; Body; Body; Bod | | | |
| | | N3; ATXN3; ATXN3; A | y; 3′UTR; Body; 3′U | | | |
| | | TXN3; ATXN3; ATXN | TR; 3′UTR; Body; B | | | |
| | | 3; ATXN3; ATXN3; AT | ody; Body; 3′UTR; 3′ | | | |
| | | XN3; ATXN3; ATXN3; | UTR; 3′UTR; Body; | | | |
| | | ATXN3; ATXN3; ATX | Body; Body; 3′UTR | | | |
| | | N3; ATXN3; ATXN3; A | | | | |
| | | TXN3 | | | | |
| 1322 | cg12052765 | SEC18A3; CHAT | TSS1500; TSS200 | N_Shore | NA | II |
| 1323 | cg12090430 | ADARB2 | Body | | NA | II |
| 1324 | cg12093519 | | | N_Shore | TRUE | II |
| 1325 | cg12103152 | HTR1B | TSS200 | Island | NA | II |
| 1326 | cg12106728 | | | Island | NA | I |
| 1327 | cg12113984 | | | Island | TRUE | II |
| 1328 | cg12116395 | ATP6AP2 | Body | S_Shore | NA | I |
| 1329 | cg12126857 | | | | TRUE | I |
| 1330 | cg12135344 | CHD5 | Body | Island | TRUE | I |
| 1331 | cg12164612 | KCNA4 | 5′UTR | Island | NA | I |
| 1332 | cg12166610 | NRG1 | Body | Island | NA | I |
| 1333 | cg12169365 | RIN3 | TSS1500 | N_Shore | NA | I |
| 1334 | cg12170314 | INSC; INSC; INSC | Body; 5′UTR; 1stEx | Island | NA | I |
| | | | on | | | |
| 1335 | cg12173409 | | | Island | NA | II |
| 1336 | cg12177793 | NFATC4; NFATC4 | Body; Body | Island | NA | 1 |
| 1337 | cg12193731 | ACVRL1 | TSS1500 | Island | NA | I |
| 1338 | cg12196692 | HEATR7B2; HEATR7 | 1stExon; 5′UTR | | NA | II |
| | | B2 | | | | |
| 1339 | cg12217600 | | | | NA | I |
| 1340 | cg12254845 | | | Island | TRUE | II |
| 1341 | cg12268236 | PRDM16; PRDM16 | Body; Body | Island | NA | I |
| 1342 | cg12287936 | NDUFS6; MRPL36 | TSS1500; TSS1500 | Island | NA | II |
| 1343 | cg12295100 | GPRASP1; GPRASP1; | 5′UTR; 5′UTR; 5′UT | Island | NA | I |
| | | GPRASP1 | R | | | |
| 1344 | cg12302982 | | | Island | NA | I |
| 1345 | cg12311175 | NDST3 | Body | | NA | II |
| 1346 | cg12323723 | | | Island | NA | I |
| 1347 | cg12337525 | | | N_Shelf | NA | II |
| 1348 | cg12341047 | FGF5; FGF5 | Body; Body | S_Shore | NA | II |
| 1349 | cg12347429 | FLJ22536 | Body | Island | NA | I |
| 1350 | cg12367389 | HTR2A; HTR2A | Body; Body | | TRUE | I |
| 1351 | cg12368188 | | | Island | NA | I |
| 1352 | cg12396306 | PCDHBI4 | TSS1500 | N_Shore | NA | I |
| 1353 | cg12403253 | ABT1 | Body | N_Shore | NA | II |
| 1354 | cg12421282 | NFYC; NFYC; LOC100 | 5UTR; 5′UTR; TSS | S_Shore | NA | II |
| | | 130557; NFYC; NFYC; | I500; 5′UTR; 5′UTR | | | |
| | | NFYC | ; 5′UTR | | | |
| 1355 | cg12444328 | LHX9; LHX9 | Body; Body | S_Shore | NA | II |
| 1356 | cg12452298 | | | N_Shore | TRUE | I |
| 1357 | cg12463578 | ZFP57 | 1stExon | | NA | I |
| 1358 | cg12467264 | | | N_Shore | TRUE | I |
| 1359 | cg12473912 | | | Island | NA | I |
| 1360 | cg12489727 | TNRC6C; TNRC6C | 3′UTR; 3′UTR | S_Shore | NA | II |
| 1361 | cg12499092 | NFYB | 3′UTR | | NA | II |
| 1362 | cg12499235 | ASCL2 | TSS1500 | S_Shore | NA | I |
| 1363 | cg12506775 | C10orf18 | 5′UTR | | TRUE | II |
| 1364 | cg12513880 | | | Island | NA | I |
| 1365 | cg12534150 | APC; APC; APC; APC; | 1stExon; 5′UTR; 1st | | TRUE | 1 |
| | | APC | Exon; 5′UTR; 5′UT | | | |
| | | | R | | | |
| 1366 | cg12563839 | C14orf159; C14orf159; | 1stExon; 1stExon; 5′ | | NA | II |
| | | C14orf159; C14orf159; | UTR; 1stExon; 5′UT | | | |
| | | C14orf159; C14orf159; | R; 5′UTR; TSS1500; | | | |
| | | C14orf159; C14orf159; | 1stExon; 5′UTR | | | |
| | | C14orf159 | | | | |
| 1367 | cg12584111 | MOXD2 | Body | | NA | I |
| 1368 | cg12584785 | CHRNA7 | Body | S_Shelf | NA | II |
| 1369 | cg12598089 | KCNAB1; KCNAB1; K | TSS200; Body; Bod | N_Shore | TRUE | II |
| | | CNAB1 | y | | | |
| 1370 | cg12610744 | KRT4 | TSS1500 | | NA | II |
| 1371 | cg12610917 | IRF2BP1 | 1stExon | Island | TRUE | II |
| 1372 | cg12620035 | | | S_Shore | NA | I |
| 1373 | cg12634080 | | | Island | NA | I |
| 1374 | cg12642693 | SHROOM4; SHROOM | 1stExon; Body | Island | NA | II |
| | | 4 | | | | |
| 1375 | cg12698813 | | | Island | NA | II |
| 1376 | cg12727795 | PDGFRB | TSS1500 | | NA | II |
| 1377 | cg12728791 | HMCN1 | Body | | NA | II |
| 1378 | cg12741994 | CLDN11 | Body | Island | TRUE | II |
| 1379 | cg12744758 | | | | NA | I |
| 1380 | cg12744820 | OLIG3 | 1stExon | Island | TRUE | I |
| 1381 | cg12754571 | LOC148824; OR2C3 | TSS200; 3′UTR | Island | NA | I |
| 1382 | cg12768605 | LYPD5 | TSS200 | Island | NA | II |
| 1383 | cg12784272 | | | | TRUE | II |
| 1384 | cg12787405 | | | Island | NA | II |
| 1385 | cg12799885 | | | Island | NA | I |
| 1386 | cg12814550 | | | S_Shore | NA | I |
| 1387 | cg12819011 | C12orf35 | Body | | TRUE | II |
| 1388 | cg12827262 | TULP1 | Body | Island | NA | II |
| 1389 | cg12829717 | DDIT4L | 5′UTR | Island | NA | I |
| 1390 | cg12832313 | PCDHAC2; PCDHAC2 | 1stExon; TSS200; B | Island | NA | I |
| | | ; PCDHA7; PCDHA12; | ody; Body; Body; Bo | | | |
| | | PCDHA6; PCDHA10; P | dy; Body; Body; Bod | | | |
| | | CDHA4; PCDHA11; P | y; Body; Body; Body | | | |
| | | CDHA8; PCDHA6; PC | ; Body; Body; Body; | | | |
| | | DHA1; PCDHA2; PCD | Body; Body; Body; 5 | | | |
| | | HA1; PCDHA9; PCDH | ′UTR; Body | | | |
| | | A13; PCDHA5; PCDH | | | | |
| | | AC1; PCDHA3; PCDH | | | | |
| | | AC2; PCDHA10 | | | | |
| 1391 | cg12833465 | SFTA3 | Body | Island | TRUE | II |
| 1392 | cg12836959 | | | | NA | I |
| 1393 | cg12847554 | SIM1 | Body | Island | NA | II |
| 1394 | cg12850379 | TCEA1; TCEA1 | TSS1500; TSS1500 | S_Shore | NA | I |
| 1395 | cg12857541 | DHX15 | Body | N_Shelf | NA | II |
| 1396 | cg12876356 | GFI1; GFI1; GFI1 | Body; Body; Body | Island | TRUE | II |
| 1397 | cg12879143 | | | Island | NA | II |
| 1398 | cg12883279 | PPT2; PRRT1; PPT2 | TSS1500; TSS1500 | N_Shore | TRUE | I |
| | | | ; TSS1500 | | | |
| 1399 | cg12898512 | TSTD1; TSTD1; TSTD | Body; 3′UTR; Body | N_Shore | NA | II |
| | | 1 | | | | |
| 1400 | cg12922751 | | | N_Shore | TRUE | II |
| 1401 | cg12938068 | | | N_Shore | NA | I |
| 1402 | cg12940439 | | | Island | NA | II |
| 1403 | cg12950441 | HTR2C | TSS200 | Island | TRUE | I |
| 1404 | cg12979234 | DBNL; DBNL; DBNL | TSS1500; TSS1500 | N_Shore | NA | II |
| | | | ; TSS1500 | | | |
| 1405 | cg13000082 | AOX1 | TSS1500 | Island | TRUE | II |
| 1406 | cg13004182 | | | Island | TRUE | I |
| 1407 | cg13012115 | OR8B2 | TSS1500 | | NA | II |
| 1408 | cg13033070 | | | Island | NA | I |
| 1409 | cg13038560 | C2orf60; C2orf60; C2or | Body; Body; TSS15 | N_Shore | NA | II |
| | | f47 | 00 | | | |
| 1410 | cg13046832 | C14orf23 | Body | Island | NA | I |
| 1411 | cg13060157 | | | Island | TRUE | I |
| 1412 | cg13062913 | NCAM2 | TSS1500 | Island | NA | II |
| 1413 | cg13097407 | CRIP3 | TSS200 | Island | TRUE | II |
| 1414 | cg13101087 | GALNT13 | 5′UTR | Island | NA | I |
| 1415 | cg13121699 | ZNF804A | TSS1500 | N_Shore | NA | II |
| 1416 | cg13164157 | PROM1; PROM1 | 5′UTR; 5′UTR | Island | TRUE | II |
| 1417 | cg13170005 | VMO1; VMO1; VMO1; | TSS1500; TSS1500 | N_Shore | NA | II |
| | | VMO1 | ; TSS1500; TSS150 | | | |
| | | | 0 | | | |
| 1418 | cg13174253 | C8orf33 | Body | S_Shore | NA | II |
| 1419 | cg13180315 | | | N_Shore | NA | I |
| 1420 | cg13181164 | MEIS2; MEIS2; MEIS2 | Body; Body; Body; | N_Shore | NA | II |
| | | ; MEIS2; MEIS2; MEIS | Body Body Body; | | | |
| | | 2; MEIS2 | Body | | | |
| 1421 | cg13200838 | | | S_Shore | TRUE | II |
| 1422 | cg13207797 | EPO | TSS200 | Island | TRUE | I |
| 1423 | cg13208922 | | | Island | NA | I |
| 1424 | cg13211683 | GDF6 | Body | Island | NA | I |
| 1425 | cg13213923 | MARCH10; MARCH1 | 5′UTR; TSS1500; 1s | Island | NA | I |
| | | 0; MARCH10 | tExon | | | |
| 1426 | cg13217661 | | | Island | NA | I |
| 1427 | cg13223227 | MAOB | Body | Island | NA | II |
| 1428 | cg13266435 | ANKRD30B | TSS200 | Island | NA | I |
| 1429 | cg13281171 | SYCP2L | TSS200 | N_Shore | NA | II |
| 1430 | cg13294846 | | | Island | NA | II |
| 1431 | cg13295238 | EBF1 | Body | Island | TRUE | II |
| 1432 | cg13300273 | GPR25 | 1stExon | Island | TRUE | I |
| 1433 | cg13300630 | ROBO1; ROBO1; ROB | Body; Body; Body; | | TRUE | II |
| | | O1; ROBO1 | Body | | | |
| 1434 | cg13340126 | SLC9A3 | Body | Island | NA | I |
| 1435 | cg13355574 | | | Island | NA | I |
| 1436 | cg13388277 | ZNF727 | TSS1500 | N_Shore | NA | II |
| 1437 | cg13406145 | | | N_Shore | TRUE | II |
| 1438 | cg13409070 | PTPRN2; PTPRN2; PT | Body; Body; Body | S_Shelf | NA | I |
| | | PRN2 | | | | |
| 1439 | cg13421439 | ANKRD29 | Body | Island | NA | I |
| 1440 | cg13434638 | ITGA11 | Body | Island | NA | I |
| 1441 | cg13443950 | | | | TRUE | II |
| 1442 | cg13449257 | | | N_Shore | NA | I |
| 1443 | cg13450196 | | | | TRUE | II |
| 1444 | cg13469595 | | | Island | TRUE | II |
| 1445 | cg13486406 | | | Island | NA | II |
| 1446 | cg13492245 | FPGS; FPGS | Body; 5′UTR | S_Shore | NA | I |
| 1447 | cg13492364 | FMN2 | Body | S_Shore | NA | II |
| 1448 | cg13501793 | | | N_Shore | NA | II |
| 1449 | cg13507224 | OR10J3 | 1stExon | | NA | II |
| 1450 | cg13525088 | | | S_Shore | TRUE | I |
| 1451 | cg13539545 | | | Island | TRUE | I |
| 1452 | cg13552831 | TRPM5 | Body | Island | NA | II |
| 1453 | cg13564460 | | | S_Shelf | NA | II |
| 1454 | cg13564742 | FOXE1 | TSS1500 | N_Shore | NA | II |
| 1455 | cg13567349 | B3GAT1; B3GAT1 | Body; Body | S_Shore | NA | II |
| 1456 | cg13568106 | | | | TRUE | I |
| 1457 | cg13582793 | NLGN1 | 5′UTR | | NA | II |
| 1458 | cg13594869 | FLRT2 | Body | | NA | II |
| 1459 | cg13611347 | NOL4 | Body | Island | TRUE | I |
| 1460 | cg13631572 | ADCY4 | TSS200 | Island | NA | I |
| 1461 | cg13631916 | FAM162B | TSS200 | Island | NA | II |
| 1462 | cg13634242 | | | Island | TRUE | I |
| 1463 | cg13644239 | TRIP13; BRD9; BRD9; | TSS1500; 5′UTR; B | N_Shore | NA | II |
| | | TRIP13 BRD9 | ody; TSS1500; Body | | | |
| 1464 | cg13655674 | TBX15 | 5′UTR | Island | NA | II |
| 1465 | cg13675050 | | | N_Shore | TRUE | I |
| 1466 | cg13678973 | ELAVL4 | Body | Island | TRUE | I |
| 1467 | cg13679303 | | | S_Shore | NA | I |
| 1468 | cg13718418 | | | N_Shore | NA | I |
| 1469 | cg13721404 | NOL4 | Body | Island | TRUE | I |
| 1470 | cg13726724 | | | Island | TRUE | II |
| 1471 | cg13748267 | | | S_Shore | NA | II |
| 1472 | cg13756380 | | | Island | NA | I |
| 1473 | cg13758712 | ADAMTS1 | TSS1500 | S_Shore | NA | I |
| 1474 | cg13799986 | TMEM141 | Body | S_Shore | NA | II |
| 1475 | cg13800209 | MEIS2; MEIS2; MEIS2 | 1stExon; Body; Bod | Island | NA | I |
| | | ; MEIS2; MEIS2; MEIS | y; 5′UTR; Body; Bod | | | |
| | | 2; MEIS2 | y; Body | | | |
| 1476 | cg13810556 | RPL37A | TSS1500 | N_Shore | NA | I |
| 1477 | cg13818316 | RIPK4 | Body | N_Shore | NA | II |
| 1478 | cg13821176 | TRIB1 | Body | S_Shelf | NA | II |
| 1479 | cg13849454 | | | Island | NA | I |
| 1480 | cg13871400 | HMSD | TSS200 | | TRUE | I |
| 1481 | cg13875120 | AOX1; AOX1 | 1stExon; 5′UTR | Island | TRUE | I |
| 1482 | cg13875536 | BARHL1 | Body | Island | TRUE | I |
| 1483 | cg13877670 | ONECUT1 | TSS1500 | Island | TRUE | II |
| 1484 | cg13878677 | IQSEC1 | Body | | TRUE | I |
| 1485 | cg13886334 | | | N_Shore | NA | II |
| 1486 | cg13897627 | | | | NA | II |
| 1487 | cg13899188 | TRIM2; TRIM2 | Body; 5′UTR | Island | NA | I |
| 1488 | cg13912117 | | | Island | NA | I |
| 1489 | cg13925692 | CHL1 | 5′UTR | Island | NA | II |
| 1490 | cg13933262 | PCDHGA4; PCDHGA | Body; Body; Body; | Island | NA | I |
| | | 9; PCDHGA1; PCDHG | Body; TSS200; Bod | | | |
| | | B1; PCDHGB7; PCDH | y; Body; TSS200; Bo | | | |
| | | GB6; PCDHGB3; PCD | dy; Body; Body; Bod | | | |
| | | HGB7; PCDHGA6; PC | y; Body; Body; Body | | | |
| | | DHGA8 ; PCDHGA 10; | ; Body; Body; Body | | | |
| | | PCDHGA5; PCDHGB4 | | | | |
| | | ; PCDHGA3; PCDHGA | | | | |
| | | 2; PCDHGB2; PCDHG | | | | |
| | | A7; PCDHGB5 | | | | |
| 1491 | cg13941830 | | | Island | NA | II |
| 1492 | cg13944838 | GFPT2 | Body | Island | NA | I |
| 1493 | cg13974838 | PTCH1; PTCH1; PTCH | Body; Body; Body; | | NA | II |
| | | 1; PTCH1; PTCH1; PTC | Body Body Body; | | | |
| | | H1; PTCH1 | Body | | | |
| 1494 | cg13990092 | OXGR1 | TSS200 | Island | TRUE | I |
| 1495 | cg13994408 | MAGEA10; MAGEA1 | TSS1500; TSS1500 | | NA | II |
| | | 0 | | | | |
| 1496 | cg14002960 | NOL4 | Body | Island | TRUE | I |
| 1497 | cg14015441 | DPYS | TSS200 | Island | NA | I |
| 1498 | cg14024242 | | | Island | NA | I |
| 1499 | cg14026459 | ELAVL2; ELAVL2; EL | 5′UTR; 5′UTR; 5′UT | Island | NA | I |
| | | AVL2 | R | | | |
| 1500 | cg14030882 | | | | NA | I |
| 1501 | cg14033514 | | | S_Shore | NA | I |
| 1502 | cg14052511 | LOC389705 | Body | | NA | II |
| 1503 | cg14056738 | | | | TRUE | I |
| 1504 | cg14064694 | HIST1H2B1 | 1stExon | Island | NA | II |
| 1505 | cg14065115 | | | N_Shore | NA | I |
| 1506 | cg14074052 | | | Island | NA | I |
| 1507 | cg14089267 | | | Island | NA | I |
| 1508 | cg14094639 | AURKAIP1 ; AURKAI | Body; Body; Body | Island | NA | I |
| | | P1; AURKAIP1 | | | | |
| 1509 | cg14094983 | NXPH1 | Body | Island | NA | I |
| 1510 | cg14126020 | C20orf166; C20orf200 | 5′UTR; TSS1500 | N_Shore | NA | I |
| 1511 | cg14142965 | TXNRD1 | TSS200 | Island | NA | I |
| 1512 | cg14145801 | ZSWIM2 | 1stExon | N_Shore | TRUE | I |
| 1513 | cg14151317 | ABCA2; ABCA2 | Body; Body | N_Shore | NA | II |
| 1514 | cg14164044 | | | N_Shore | TRUE | II |
| 1515 | cg14179389 | GFT1; GFT1; GFT 1 | Body; Body; Body | Island | NA | I |
| 1516 | cg14189141 | | | Island | NA | I |
| 1517 | cg14192029 | ZNF718 | Body | S_Shore | NA | II |
| 1518 | cg14202679 | | | S_Shore | NA | II |
| 1519 | cg14203525 | GAP43; GAP43 | Body; 5′UTR | Island | NA | I |
| 1520 | cg14206172 | FBXO17 | TSS1500 | S_Shore | TRUE | I |
| 1521 | cg14207921 | KLHL33 | Body | | NA | I |
| 1522 | cg14238325 | CRIP3 | TSS1500 | S_Shore | NA | II |
| 1523 | cg14247635 | ZAP70; ZAP70 | 3′UTR; 3′UTR | | NA | II |
| 1524 | cg14248367 | | | | TRUE | I |
| 1525 | cg14250833 | GCM2 | TSS200 | N_Shore | NA | I |
| 1526 | cg14254720 | IRRC8C | TSS1500 | N_Shore | NA | II |
| 1527 | cg14261840 | FEZF2 | TSS1500 | S_Shore | NA | II |
| 1528 | cg14262681 | | | N_Shore | NA | I |
| 1529 | cg14273822 | HIST1H3G ; HIST1H2 | TSS200; TSS1500 | N_Shore | NA | II |
| | | BI | | | | |
| 1530 | cg14283569 | | | Island | NA | I |
| 1531 | cg14283758 | | | S_Shore | NA | II |
| 1532 | cg14299940 | PHYTID1; PHYTID1; P | Body; Body; Body | | NA | I |
| | | HYHD1 | | | | |
| 1533 | cg14300812 | MED27 | Body | | TRUE | II |
| 1534 | cg14323199 | MRC2 | Body | Island | NA | II |
| 1535 | cg14326671 | LOC100192378 | Body | | TRUE | I |
| 1536 | cg14343214 | EYA4; EYA4; EYA4 | TSS1500; TSS1500 | N_Shore | NA | I |
| | | | ; TSS1500 | | | |
| 1537 | cg14348741 | | | Island | NA | I |
| 1538 | cg14349667 | FGF5; FGF5 | Body; Body | S_Shore | NA | I |
| 1539 | cg14362370 | ABE1; ABE1 | 5′UTR; 1stExon | S_Shore | NA | II |
| 1540 | cg14376548 | CNP | Body | Island | NA | II |
| 1541 | cg14380270 | SLFN11; SLFN11; SLF | TSS200; TSS200; T | Island | TRUE | I |
| | | N11; SLFN11; SLFN11 | SS200; TSS200; TS | | | |
| | | | S200 | | | |
| 1542 | cg14395298 | | | Island | NA | I |
| 1543 | cg14399930 | FBXO47 | TSS200 | | NA | I |
| 1544 | cg14400354 | GALNT12 | Body | S_Shore | NA | I |
| 1545 | cg14409023 | OTX2OS1 | Body | N_Shore | NA | I |
| 1546 | cg14428146 | NKX2-6 | TSS200 | Island | TRUE | I |
| 1547 | cg14448830 | | | N_Shore | NA | I |
| 1548 | cg14454796 | MTERFD2; MTERFD2 | Body; Body; 3′UTR; | | NA | I |
| | | ; SNED1; MTERFD2 | Body | | | |
| 1549 | cg14454907 | | | N_Shelf | NA | II |
| 1550 | cg14478768 | HMHA1 | Body | Island | NA | II |
| 1551 | cg14480116 | SPRED2; SPRED2 | TSS1500; Body | | NA | II |
| 1552 | cg14482313 | KRT7 | TSS200 | Island | NA | I |
| 1553 | cg14503815 | | | | TRUE | II |
| 1554 | cg14511739 | APC; APC; APC | TSS200; TSS200; 5′ | | NA | I |
| | | | UTR | | | |
| 1555 | cg14514403 | ARX | TSS200 | Island | NA | I |
| 1556 | cg14515517 | MYO3A | 5′UTR | S_Shelf | NA | II |
| 1557 | cg14537713 | | | N_Shore | NA | I |
| 1558 | cg14556896 | | | Island | NA | II |
| 1559 | cg14557185 | WWTR1; WWTR1; W | Body; Body; Body | Island | NA | I |
| | | WTR1 | | | | |
| 1560 | cg14575053 | HMSD | TSS200 | | TRUE | I |
| 1561 | cg14576896 | GJD2 | 1stExon | Island | NA | I |
| 1562 | cg14578879 | SIM1 | Body | N_Shore | TRUE | II |
| 1563 | cg14603098 | ACCN1 | TSS1500 | Island | NA | I |
| 1564 | cg14604568 | PDE1C | 5′UTR | Island | NA | I |
| 1565 | cg14605874 | | | N_Shore | NA | II |
| 1566 | cg14611399 | | | N_Shore | TRUE | I |
| 1567 | cg14613540 | HMSD | TSS1500 | | TRUE | II |
| 1568 | cg14616251 | GRHL3; GRHL3; GRH | TSS1500; Body; TS | Island | NA | II |
| | | L3 | S1500 | | | |
| 1569 | cg14618996 | ADAM32 | TSS200 | N_Shore | NA | II |
| 1570 | cg14633455 | ZFP37 | TSS200 | Island | NA | I |
| 1571 | cg14642432 | A2BP1; A2BP1 | 5′UTR; 5′UTR | Island | NA | II |
| 1572 | cg14643001 | | | N_Shelf | TRUE | II |
| 1573 | cg14643390 | | | | TRUE | II |
| 1574 | cg14643537 | | | S_Shore | NA | II |
| 1575 | cg14646111 | SEC23B; SEC23B; SEC | 5′UTR; 5′UTR; 5′UT | S_Shore | NA | I |
| | | 23B | R | | | |
| 1576 | cg14651249 | BARHL1 | 3′UTR | Island | TRUE | II |
| 1577 | cg14651254 | ATAD3A; ATAD3A; A | 5′UTR; Body; Body | S_Shore | NA | I |
| | | TAD3A | | | | |
| 1578 | cg14663264 | | | Island | NA | I |
| 1579 | cg14666564 | HOXD3 | 5′UTR | S_Shore | NA | I |
| 1580 | cg14686949 | GABRE; MIR452 | Body; TSS1500 | | NA | II |
| 1581 | cg14696535 | HIST1H3G; HIST1H2 | TSS1500; TSS200 | N_Shore | NA | II |
| | | BI | | | | |
| 1582 | cg14700679 | | | | TRUE | I |
| 1583 | cg14714797 | AHRR | Body | Island | NA | I |
| 1584 | cg14717557 | SIM1 | Body | N_Shore | TRUE | II |
| 1585 | cg14759284 | SRPX; SRPX; SRPX; S | TSS200; TSS200; T | S_Shore | NA | II |
| | | RPX | 55200; TSS200 | | | |
| 1586 | cg14762784 | ATF6B; ATF6B; FKBP | TSS1500; TSS1500 | N_Shore | NA | II |
| | | L | ; Body | | | |
| 1587 | cg14762973 | ZSWIM2 | TSS200 | Island | TRUE | II |
| 1588 | cg14765959 | | | Island | NA | I |
| 1589 | cg14776962 | HIST1H2BH; HIST1H | 1stExon; TSS1500 | Island | NA | II |
| | | 3F | | | | |
| 1590 | cg14781189 | KCNH7; KCNH7; KCN | 1stExon; 5′UTR; 1st | | NA | I |
| | | H7; KCNH7 | Exon; 5′UTR | | | |
| 1591 | cg14808890 | HIST1H2BH; HIST1H | TSS1500; 1stExon | Island | NA | II |
| | | 3F | | | | |
| 1592 | cg14817655 | ZFP42 | TSS200 | Island | NA | I |
| 1593 | cg14824495 | SOX15 | 1stExon | Island | NA | II |
| 1594 | cg14825976 | C18orf1; C18orf1; C18o | TSS1500; Body; Bo | | TRUE | II |
| | | rf1; C18orf1 | dy; TSS1500 | | | |
| 1595 | cg14826331 | CNP | Body | S_Shore | NA | I |
| 1596 | cg14843688 | | | | TRUE | II |
| 1597 | cg14846965 | C18orf1; C18orf1; C18o | TSS200; Body; Bod | | TRUE | I |
| | | rf1; C18orf1 | y; TSS200 | | | |
| 1598 | cg14869505 | ATP5B; SNORD59B | Body; TSS200 | N_Shore | NA | II |
| 1599 | cg14871932 | GCK; GCK; GCK | Body; Body; Body | Island | NA | I |
| 1600 | cg14883291 | ME3; ME3; ME3 | TSS1500; TSS200; | S_Shore | NA | II |
| | | | TSS1500 | | | |
| 1601 | cg14885748 | | | Island | TRUE | I |
| 1602 | cg14889643 | BCL2; BCL2 | Body; Body | Island | NA | I |
| 1603 | cg14903097 | C2orf43 | TSS1500 | S_Shore | NA | II |
| 1604 | cg14914046 | | | | TRUE | II |
| 1605 | cg14926717 | FLJ43390 | TSS200 | Island | NA | I |
| 1606 | cg14934253 | | | N_Shore | NA | II |
| 1607 | cg14943113 | | | S_Shore | TRUE | II |
| 1608 | cg14951132 | NRK | Body | Island | NA | I |
| 1609 | cg14959260 | SLC2A9; SLC2A9 | Body; Body | | NA | II |
| 1610 | cg14961598 | MTMR8 | Body | N_Shore | NA | I |
| 1611 | cg14962032 | CASP8; CASP8; CASP | 5′UTR; 5′UTR; 5′UT | | TRUE | II |
| | | 8; CASP8; CASP8 | R; 5′UTR; TSS200 | | | |
| 1612 | cg14968543 | | | Island | NA | II |
| 1613 | cg14989988 | ATXN7L1; ATXN7L1 | Body; TSS1500 | | TRUE | II |
| 1614 | cg14996220 | ALX1 | TSS1500 | N_Shore | NA | I |
| 1615 | cg14997942 | | | | TRUE | II |
| 1616 | cg15012484 | | | Island | NA | I |
| 1617 | cg15013801 | ASCC1; C10orf104 | TSS1500; 5′UTR | S_Shore | NA | I |
| 1618 | cg15026277 | TMEM106A | TSS200 | Island | NA | I |
| 1619 | cg15028507 | C7orf50; C7orf50; C7or | Body; Body; Body | S_Shore | NA | I |
| | | f50 | | | | |
| 1620 | cg15035437 | | | | NA | I |
| 1621 | cg15043384 | WWTR1; WWTR1; W | Body; Body; Body | Island | NA | I |
| | | WTR1 | | | | |
| 1622 | cg15054725 | | | Island | NA | I |
| 1623 | cg15056128 | KRT222 | TSS200 | | NA | II |
| 1624 | cg15059608 | C3orf14 | TSS1500 | S_Shore | NA | II |
| 1625 | cg15060599 | | | Island | NA | I |
| 1626 | cg15077070 | IL1RL2 | TSS200 | N_Shore | NA | II |
| 1627 | cg15080119 | HIST1H2BH; HIST1H | 3′UTR; 1stExon; TS | S_Shore | NA | II |
| | | 2BH; HIST1H3F | S1500 | | | |
| 1628 | cg15089705 | | | N_Shore | NA | II |
| 1629 | cg15095922 | NDUFS6; MRPL36 | TSS1500; TSS1500 | Island | NA | I |
| 1630 | cg15114474 | | | Island | TRUE | I |
| 1631 | cg15131808 | | | Island | NA | I |
| 1632 | cg15132013 | SKI | Body | S_Shelf | NA | II |
| 1633 | cg15133018 | | | Island | NA | I |
| 1634 | cg15140465 | PAK1; PAK1 | 5′UTR; 5′UTR | Island | NA | II |
| 1635 | cg15141467 | EFNA4; EFNA4; EFNA | TSS1500; TSS1500 | Island | NA | I |
| | | 4 | ; TSS1500 | | | |
| 1636 | cg15160263 | | | Island | NA | I |
| 1637 | cg15162392 | QPCT | TSS200 | Island | TRUE | I |
| 1638 | cg15169940 | TBCB; POLR2I | TSS1500; 3′UTR | Island | NA | I |
| 1639 | cg15177902 | FBXO17; FBXO17 | 5′UTR; 1stExon | Island | TRUE | II |
| 1640 | cg15203028 | MUC6 | Body | Island | NA | I |
| 1641 | cg15215612 | | | Island | NA | I |
| 1642 | cg15219228 | TULP1 | Body | Island | TRUE | I |
| 1643 | cg15243542 | PURB; PURB | 3′UTR; 1stExon | | NA | II |
| 1644 | cg15265102 | | | N_Shore | NA | II |
| 1645 | cg15266705 | | | Island | NA | I |
| 1646 | cg15267232 | GATA3; GATA3 | Body; Body | Island | NA | I |
| 1647 | cg15285250 | NXPH1 | Body | Island | NA | II |
| 1648 | cg15291243 | LHX1 | Body | Island | NA | II |
| 1649 | cg15292275 | | | Island | NA | II |
| 1650 | cg15339075 | PARK2; PARK2; PAR | Body; Body; Body | | TRUE | II |
| | | K2 | | | | |
| 1651 | cg15345437 | SNORD22; SNORD31; | TSS1500; Body; Bo | N_Shelf | NA | II |
| | | SNHG1 | dy | | | |
| 1652 | cg15368905 | HTR1B | 1stExon | Island | NA | I |
| 1653 | cg15375469 | CPVL; CPVL | TSS200; TSS200 | | TRUE | II |
| 1654 | cg15379185 | EVX1 | 1stExon | Island | TRUE | I |
| 1655 | cg15386434 | MXRA8 | Body | Island | TRUE | I |
| 1656 | cg15386964 | HIST1H2BH; HIST1H | TSS1500; 1stExon | Island | NA | I |
| | | 3F | | | | |
| 1657 | cg15392549 | | | | TRUE | II |
| 1658 | cg15394142 | HMGA2; HMGA2 | Body; Body | | TRUE | II |
| 1659 | cg15398152 | TNXB | Body | | NA | II |
| 1660 | cg15400655 | ZNF718 | Body | S_Shore | NA | I |
| 1661 | cg15401418 | SEPT9; SEPT9; SEPT9; | 5′UTR; 5′UTR; Bod | | TRUE | I |
| | | SEPT9 | y; 1stExon | | | |
| 1662 | cg15412759 | TFAP2D | Body | Island | NA | II |
| 1663 | cg15420720 | STC2 | Body | N_Shelf | TRUE | II |
| 1664 | cg15423357 | | | | TRUE | I |
| 1665 | cg15436123 | RGS12; RGS12; RGS12 | Body; Body; Body | | TRUE | I |
| 1666 | cg15442728 | C14orf159; C14orf159; | TSS200; TSS200; T | | NA | II |
| | | C14orf159; C14orf159; | SS200; TSS200; TS | | | |
| | | C14orf159 | S1500 | | | |
| 1667 | cg15445000 | MED1 | TSS1500 | S_Shore | NA | II |
| 1668 | cg15447787 | | | Island | NA | I |
| 1669 | cg15455643 | COL4A2 | Body | S_Shore | NA | II |
| 1670 | cg15465051 | | | | TRUE | II |
| 1671 | cg15468095 | PYCARD; PYCARD | TSS200; TSS200 | S_Shore | NA | II |
| 1672 | cg15473084 | | | Island | TRUE | II |
| 1673 | cg15496063 | CYS1 | TSS1500 | Island | NA | II |
| 1674 | cg15496864 | | | Island | TRUE | II |
| 1675 | cg15504461 | | | N_Shore | NA | II |
| 1676 | cg15511022 | | | N_Shelf | NA | II |
| 1677 | cg15523060 | MBD3L1 | TSS1500 | | NA | II |
| 1678 | cg15534561 | | | | NA | II |
| 1679 | cg15580075 | NCRNA00167; PRDM | TSS1500; 5′UTR; 5′ | N_Shore | NA | II |
| | | 10; PRDM10 | UTR | | | |
| 1680 | cg15595525 | GPR123 | 3′UTR | N_Shore | NA | II |
| 1681 | cg15610027 | DTNA; DTNA; DTNA; | TSS200; 5′UTR; 5′U | | TRUE | II |
| | | DTNA | TR; 5′UTR | | | |
| 1682 | cg15611912 | RGS10 | Body | N_Shore | NA | II |
| 1683 | cg15621260 | FIBIN; FIBIN | 5′UTR; 1stExon | | TRUE | I |
| 1684 | cg15654259 | | | Island | TRUE | I |
| 1685 | cg15680288 | | | | TRUE | I |
| 1686 | cg15680620 | ANKRD45 | TSS200 | S_Shore | NA | II |
| 1687 | cg15682101 | | | S_Shelf | NA | II |
| 1688 | cg15727252 | SYT11 | Body | S_Shore | NA | II |
| 1689 | cg15732851 | ATP2B4; ATP2B4 | 5′UTR; 5′UTR | Island | NA | I |
| 1690 | cg15754153 | PLEKHH2 | 5′UTR | S_Shore | NA | II |
| 1691 | cg15759056 | ABCB1 | 5′UTR | Island | TRUE | I |
| 1692 | cg15778054 | KCTD14 | TSS200 | | NA | I |
| 1693 | cg15779837 | GRIN2D | Body | Island | TRUE | I |
| 1694 | cg15790037 | ARL5C | Body | Island | NA | I |
| 1695 | cg15814504 | | | Island | NA | II |
| 1696 | cg15827779 | CDC42BPB | TSS1500 | S_Shore | NA | I |
| 1697 | cg15843567 | MYO3A; MYO3A | 1stExon; 5′UTR | Island | NA | I |
| 1698 | cg15850390 | CDX4 | Body | S_Shore | TRUE | I |
| 1699 | cg15866977 | NOTUM | TSS1500 | Island | NA | I |
| 1700 | cg15874629 | MAOB; MAOB | 1stExon; 5′UTR | Island | NA | I |
| 1701 | cg15904664 | ASPG | Body | Island | NA | I |
| 1702 | cg15910208 | KLK10; KEK10; KLK1 | 5′UTR; 5′UTR; 5′UT | Island | NA | II |
| | | 0 | R | | | |
| 1703 | cg15916192 | | | Island | TRUE | II |
| 1704 | cg15920791 | | | Island | NA | I |
| 1705 | cg15932013 | FLJ22536 | TSS200 | Island | NA | I |
| 1706 | cg15952370 | OR8D2 | TSS1500 | | NA | II |
| 1707 | cg15953310 | | | Island | TRUE | I |
| 1708 | cg15959715 | GAD2; GAD2 | Body; Body | Island | NA | I |
| 1709 | cg15963552 | C3orf26; FILIP1L; MIR | Body; Body; Body; 1 | N_Shore | NA | I |
| | | 548G; FILIP1L; FILIP1 | stExon; 5′UTR; Bod | | | |
| | | L; FILIP1L | y | | | |
| 1710 | cg15965006 | STK19; STK19; STK19 | TSS200; TSS1500; | N_Shore | NA | I |
| | | ; DOM3Z | TSS1500; Body | | | |
| 1711 | cg15972331 | FGF14; FGF14 | Body; TSS1500 | S_Shore | NA | I |
| 1712 | cg15973954 | | | | NA | II |
| 1713 | cg15982972 | | | | NA | II |
| 1714 | cg15984661 | CCDC8; CCDC8 | 1stExon; 5′UTR | N_Shore | NA | II |
| 1715 | cg15992535 | NRG2; NRG2; NRG2; N | Body; Body; Body; | Island | NA | I |
| | | RG2 | Body | | | |
| 1716 | cg15994026 | CD38 | Body | Island | TRUE | I |
| 1717 | cg16045423 | APOBEC3B | TSS1500 | | TRUE | II |
| 1718 | cg16072304 | | | S_Shore | NA | II |
| 1719 | cg16101148 | | | S_Shore | NA | II |
| 1720 | cg16111259 | MAGI2 | Body | | TRUE | II |
| 1721 | cg16117799 | PRDM8 | 5′UTR | S_Shore | NA | I |
| 1722 | cg16132520 | AKRIB1 | TSS200 | Island | NA | I |
| 1723 | cg16133088 | EFCAB4B; EFCAB4B; | TSS200; TSS200; T | Island | TRUE | I |
| | | EFCAB4B | SS200 | | | |
| 1724 | cg16167741 | NFYC; NFYC; NFYC; | 5′UTR; 5′UTR; 5′UT | | NA | II |
| | | NFYC; NFYC | R; 5′UTR; 5′UTR | | | |
| 1725 | cg16182936 | IQSEC1 | Body | | TRUE | I |
| 1726 | cg16195091 | LOC283999 | Body | N_Shore | NA | I |
| 1727 | cg16201038 | CDX2 | TSS1500 | Island | TRUE | I |
| 1728 | cg16201883 | FSCN2; FSCN2 | 1stExon; 1stExon | Island | NA | I |
| 1729 | cg16215203 | ADCY4 | TSS200 | Island | NA | I |
| 1730 | cg16246242 | | | | TRUE | II |
| 1731 | cg16258337 | | | N_Shore | TRUE | II |
| 1732 | cg16259229 | | | | NA | II |
| 1733 | cg16289929 | | | N_Shore | NA | II |
| 1734 | cg16296754 | | | N_Shore | TRUE | II |
| 1735 | cg16300300 | | | N_Shore | NA | I |
| 1736 | cg16303918 | FEV | Body | N_Shore | NA | II |
| 1737 | cg16312163 | ALDH1L1; ALDH1L1 | 5′UTR; 1stExon | Island | TRUE | II |
| 1738 | cg16322479 | EXOC3; C5orf55 | 5′UTR; TSS1500 | S_Shore | NA | II |
| 1739 | cg16329197 | | | Island | TRUE | II |
| 1740 | cg16332224 | MOCS3; DPM1 | 1stExon; TSS1500 | S_Shore | NA | 11 |
| 1741 | cg16354520 | LCE2A | 3′UTR | | NA | II |
| 1742 | cg16357179 | DKFZp434L192 | TSS1500 | | NA | II |
| 1743 | cg16366686 | SEPT9; SEPT9; SEPT9; | 5′UTR; 5′UTR; Bod | | TRUE | II |
| | | SEPT9 | y; 1stExon | | | |
| 1744 | cg16373000 | C7orf50; C7orf50; C7or | Body; Body; Body | S_Shore | NA | II |
| | | f50 | | | | |
| 1745 | cg16377609 | | | S_Shore | NA | II |
| 1746 | cg16382584 | 11-Sep | Body | | TRUE | II |
| 1747 | cg16401529 | PABPC5; PABPC5 | 5′UTR; 1stExon | Island | NA | I |
| 1748 | cg16406225 | | | N_Shore | TRUE | II |
| 1749 | cg16413687 | ALX1 | TSS1500 | N_Shore | NA | I |
| 1750 | cg16413715 | UNC5A | Body | Island | NA | II |
| 1751 | cg16414561 | ARX; ARX | 1stExon; 5′UTR | Island | NA | I |
| 1752 | cg16448058 | TLX1NB | 5′UTR | N_Shelf | NA | I |
| 1753 | cg16459265 | C7orf40; SNORA9 | Body; Body | N_Shore | NA | II |
| 1754 | cg16462006 | | | | TRUE | II |
| 1755 | cg16469740 | HDAC9; HDAC9 | Body; Body | | TRUE | II |
| 1756 | cg16473141 | REC8; REC8; REC8; RE | 1stExon; 1stExon; 5′ | Island | TRUE | II |
| | | C8 | UTR; 5′UTR | | | |
| 1757 | cg16475478 | | | | NA | II |
| 1758 | cg16490191 | UNKL | 5′UTR | N_Shore | TRUE | II |
| 1759 | cg16507965 | ARX | TSS200 | Island | NA | I |
| 1760 | cg16509851 | | | Island | NA | I |
| 1761 | cg16515238 | FAM70A; FAM70A; F | Body; Body; Body | Island | NA | I |
| | | AM70A | | | | |
| 1762 | cg16524733 | TAGEN; TAGEN; TAG | 5′UTR; 1stExon; 5′U | | NA | I |
| | | EN; TAGEN | TR; 1stExon | | | |
| 1763 | cg16530382 | FBXO47 | TSS200 | | NA | II |
| 1764 | cg16539957 | | | N_Shore | NA | II |
| 1765 | cg16559570 | RTEL1; RTEL1 | Body; Body | Island | NA | I |
| 1766 | cg16565394 | SIM1 | Body | N_Shore | NA | II |
| 1767 | cg16570157 | EFCAB4A | Body | Island | NA | I |
| 1768 | cg16589135 | | | Island | NA | I |
| 1769 | cg16590821 | LONRF3; LONRF3 | TSS1500; TSS1500 | Island | NA | I |
| 1770 | cg16596691 | SNRPA | Body | S_Shelf | NA | II |
| 1771 | cg16618605 | GABRE; MIR452 | Body; TSS200 | | NA | II |
| 1772 | cg16620382 | TTBK1 | TSS200 | Island | NA | I |
| 1773 | cg16640855 | NEUROD1 | Body | | TRUE | I |
| 1774 | cg16643088 | CSRNP1 | 5′UTR | S_Shelf | TRUE | II |
| 1775 | cg16653620 | | | Island | NA | II |
| 1776 | cg16664405 | | | Island | NA | I |
| 1777 | cg16675381 | | | | NA | II |
| 1778 | cg16683770 | | | Island | TRUE | I |
| 1779 | cg16692973 | FAM70A; FAM70A; F | 1stExon; 5′UTR; 5′U | Island | NA | I |
| | | AM70A; FAM70A; FA | TR; 1stExon; 1stExo | | | |
| | | M70A; FAM70A | n; 5′UTR | | | |
| 1780 | cg16695483 | NID2 | TSS1500 | Island | NA | I |
| 1781 | cg16705697 | | | | NA | I |
| 1782 | cg16709294 | SFRS5; SFRS5; LOC10 | Body; Body; TSS15 | S_Shore | NA | I |
| | | 028951 | 00 | | | |
| 1783 | cg16718276 | C1orf86; LOC1001280 | Body; TSS200; Bod | N_Shore | NA | I |
| | | 03; C1orf86 | y | | | |
| 1784 | cg16718624 | ROBO2; ROBO2 | 1stExon; 5′UTR | S_Shore | NA | I |
| 1785 | cg16732616 | DMRTA2 | Body | Island | NA | I |
| 1786 | cg16733705 | | | | NA | I |
| 1787 | cg16739441 | KCNJ8 | Body | | NA | II |
| 1788 | cg16744973 | | | N_Shelf | NA | II |
| 1789 | cg16747109 | PITX2; PITX2; PITX2 | TSS1500; Body; Bo | Island | NA | II |
| | | | dy | | | |
| 1790 | cg16749578 | NR2F6 | TSS1500 | S_Shore | NA | II |
| 1791 | cg16763089 | LOC149837 | TSS200 | | NA | II |
| 1792 | cg16768018 | ZIC4; ZIC4; ZIC4; ZIC4 | Body; Body; Body; | Island | NA | I |
| | | ; ZIC4 | Body; Body | | | |
| 1793 | cg16772207 | MYT1 | TSS200 | | NA | II |
| 1794 | cg16773115 | TPD52; TPD52; TPD52 | Body; 5′UTR; 1stEx | | TRUE | II |
| | | ; TPD52 | on; Body | | | |
| 1795 | cg16773899 | EDIL3; EDIL3 | 5′UTR; 1stExon | Island | NA | II |
| 1796 | cg16800165 | FLRT2 | TSS200 | N_Shore | NA | II |
| 1797 | cg16803596 | GATA4 | TSS1500 | Island | TRUE | I |
| 1798 | cg16804284 | FOXG1 | 1stExon | Island | NA | II |
| 1799 | cg16819028 | PKP1; PKP1 | TSS1500; TSS1500 | N_Shore | NA | II |
| 1800 | cg16831623 | BMP2 | 5′UTR | N_Shore | NA | II |
| 1801 | cg16833110 | OR2D3 | TSS200 | | NA | II |
| 1802 | cg16842053 | CARD11; CARD11 | 1stExon; 5′UTR | | TRUE | I |
| 1803 | cg16855845 | DNASE2 | Body | N_Shore | NA | II |
| 1804 | cg16869108 | VHL; VHL | Body; Body | S_Shore | NA | II |
| 1805 | cg16894819 | | | N_Shelf | NA | II |
| 1806 | cg16927242 | MEIS1 | Body | S_Shelf | NA | II |
| 1807 | cg16968020 | GABRA3 | Body | | NA | II |
| 1808 | cg16998564 | | | | TRUE | II |
| 1809 | cg17004353 | CARS; CARS; CARS; C | Body; Body; 5′UTR; | | NA | I |
| | | ARS | Body | | | |
| 1810 | cg17032646 | SLC6A17 | TSS1500 | N_Shore | TRUE | II |
| 1811 | cg17034390 | | | Island | NA | I |
| 1812 | cg17052266 | CPVL; CPVL | TSS200; TSS200 | | TRUE | II |
| 1813 | cg17071446 | | | N_Shore | TRUE | II |
| 1814 | cg17080882 | TGFBR3 | Body | | TRUE | II |
| 1815 | cg17084751 | ZNF215 | Body | | NA | II |
| 1816 | cg17093995 | VWC2 | Body | Island | NA | I |
| 1817 | cg17094985 | | | Island | TRUE | I |
| 1818 | cg17098995 | CAT | 3′UTR | | NA | I |
| 1819 | cg17099568 | | | N_Shore | NA | II |
| 1820 | cg17100761 | EPO | TSS1500 | N_Shore | TRUE | I |
| 1821 | cg17101165 | | | Island | NA | II |
| 1822 | cg17105834 | ASTN1; ASTN1 | 1stExon; 1stExon | Island | TRUE | I |
| 1823 | cg17109725 | | | S_Shelf | NA | II |
| 1824 | cg17110524 | | | Island | NA | II |
| 1825 | cg17160382 | GJA1 | 5′UTR | | NA | I |
| 1826 | cg17160886 | C6orf141 | 1stExon | Island | TRUE | II |
| 1827 | cg17163760 | CPVL; CPVL | TSS200; TSS200 | | TRUE | II |
| 1828 | cg17177455 | | | Island | NA | II |
| 1829 | cg17201760 | ZNF876P | Body | Island | NA | II |
| 1830 | cg17202313 | SOX2OT | Body | Island | NA | I |
| 1831 | cg17214381 | ZFP42 | TSS1500 | Island | NA | I |
| 1832 | cg17214408 | | | | NA | I |
| 1833 | cg17235702 | | | Island | NA | II |
| 1834 | cg17237063 | RBMS3; RBMS3; RBM | 1stExon; 1stExon; 1 | | NA | II |
| | | S3 | stExon | | | |
| 1835 | cg17241776 | DLEU7 | 1stExon | Island | NA | I |
| 1836 | cg17242351 | ACTC1 | Body | | NA | I |
| 1837 | cg17266581 | MBP; MBP | Body; Body | Island | NA | I |
| 1838 | cg17276590 | EMX2OS; EMX2; EM | Body; Body; Body | Island | NA | I |
| | | X2 | | | | |
| 1839 | cg17280346 | ZIC1 | TSS1500 | N_Shore | NA | I |
| 1840 | cg17284804 | TCTEX1D1 | TSS200 | Island | TRUE | I |
| 1841 | cg17292384 | COXA | 3′UTR | S_Shore | NA | II |
| 1842 | cg17302155 | PRDM13 | Body | Island | NA | I |
| 1843 | cg17305181 | CRH; CRH | 1stExon; 5′UTR | S_Shore | NA | I |
| 1844 | cg17315500 | | | Island | TRUE | I |
| 1845 | cg17332573 | HIST1H2BH; HIST1H | TSS1500; TSS200 | N_Shore | NA | II |
| | | 3F | | | | |
| 1846 | cg17342759 | PPP2R2B; PPP2R2B | TSS1500; TSS1500 | | NA | II |
| 1847 | cg17344516 | | | Island | TRUE | I |
| 1848 | cg17361163 | EMID2 | TSS200 | Island | NA | II |
| 1849 | cg17374727 | LUZP6; MTPN | 5′UTR; Body | | TRUE | II |
| 1850 | cg17380661 | SIM1 | TSS1500 | Island | NA | I |
| 1851 | cg17385175 | | | Island | NA | I |
| 1852 | cg17392573 | | | Island | NA | I |
| 1853 | cg17394649 | HCG4 | Body | Island | NA | I |
| 1854 | cg17407511 | SRPX; SRPX; SRPX; S | TSS200; TSS200; T | S_Shore | NA | II |
| | | RPX | SS200; TSS200 | | | |
| 1855 | cg17410236 | FIRT2; FIRT2 | 1stExon; 5′UTR | Island | NA | I |
| 1856 | cg17435901 | SLC39A7; RXRB; SLC | Body; TSS1500; Bo | S_Shore | NA | I |
| | | 39A7 | dy | | | |
| 1857 | cg17440085 | | | | NA | II |
| 1858 | cg17448336 | | | N_Shore | NA | II |
| 1859 | cg17499941 | LOC645323 | Body | N_Shore | NA | II |
| 1860 | cg17529435 | | | Island | NA | I |
| 1861 | cg17544952 | MEOX2 | Body | | NA | II |
| 1862 | cg17546247 | ZIC4; ZIC1 | TSS1500; TSS1500 | N_Shore | TRUE | II |
| 1863 | cg17555825 | | | Island | NA | I |
| 1864 | cg17559156 | SLC43A1 | Body | S_Shelf | NA | II |
| 1865 | cg17560340 | RNMTL1; GLOD4 | TSS1500; Body | N_Shore | NA | II |
| 1866 | cg17562247 | FAM70A; FAM70A; F | Body; Body; Body | N_Shore | NA | II |
| | | AM70A | | | | |
| 1867 | cg17569842 | RGS12; RGS12; RGS12 | Body; TSS200; Bod | N_Shelf | TRUE | II |
| | | | y | | | |
| 1868 | cg17570276 | EIF2AK1; EIF2AK1 | TSS1500; TSS1500 | S_Shore | NA | I |
| 1869 | cg17594860 | AKR7A3 | TSS200 | Island | NA | I |
| 1870 | cg17600231 | TSC22D1; TSC22D1 | 3′UTR; 3′UTR | N_Shelf | TRUE | II |
| 1871 | cg17602451 | BCL2; BCL2 | Body; Body | Island | NA | II |
| 1872 | cg17608490 | FEV | Body | N_Shore | NA | II |
| 1873 | cg17609804 | TBRG4; TBRG4; TBR | Body; Body; Body | N_Shelf | NA | II |
| | | G4 | | | | |
| 1874 | cg17618327 | MAD1L1; MAD1L1; M | Body; Body; Body | | TRUE | I |
| | | AD1L1 | | | | |
| 1875 | cg17630392 | NPR3 | 1stExon | Island | NA | II |
| 1876 | cg17632299 | LECT1; LECT1 | Body; Body | N_Shore | TRUE | I |
| 1877 | cg17641904 | C9orf4 | 1stExon | Island | NA | I |
| 1878 | cg17647537 | HTR3B | Body | | NA | II |
| 1879 | cg17647884 | GNRH1; GNRH1 | TSS200; TSS200 | | NA | II |
| 1880 | cg17665652 | ANK2; ANK2; ANK2 | Body; Body; Body | | TRUE | II |
| 1881 | cg17675712 | | | | TRUE | II |
| 1882 | cg17683110 | | | Island | NA | II |
| 1883 | cg17684207 | LOC285796 | TSS1500 | | NA | I |
| 1884 | cg17684657 | SYCP2L | TSS200 | N_Shore | NA | II |
| 1885 | cg17696160 | PAX9 | Body | Island | NA | II |
| 1886 | cg17705066 | GIPC3 | Body | Island | NA | I |
| 1887 | cg17711567 | LYPD5 | TSS1500 | Island | NA | II |
| 1888 | cg17717124 | TCL1A; TCL1A | TSS1500; TSS1500 | Island | NA | II |
| 1889 | cg17718302 | HIST1H3J | TSS200 | S_Shore | NA | II |
| 1890 | cg17720013 | IGFE4 | TSS1500 | | NA | II |
| 1891 | cg17721249 | ATP2B4; ATP2B4 | 5′UTR; 5′UTR | Island | TRUE | I |
| 1892 | cg17722002 | | | Island | NA | II |
| 1893 | cg17739038 | HIC1 | TSS1500 | Island | NA | I |
| 1894 | cg17740140 | TOP1 | Body | S_Shore | NA | II |
| 1895 | cg17759535 | NR5A2; NR5A2 | Body; Body | N_Shore | NA | II |
| 1896 | cg17795240 | GATA4 | Body | S_Shore | NA | II |
| 1897 | cg17797229 | | | S_Shore | TRUE | II |
| 1898 | cg17800442 | CTF1; CTF1 | Body; Body | S_Shelf | NA | II |
| 1899 | cg17804356 | NR5A2; NR5A2 | Body; Body | Island | NA | II |
| 1900 | cg17816394 | GNG4; GNG4; GNG4; | TSS1500; 1stExon; | Island | NA | I |
| | | GNG4; GNG4 | 5′UTR; 1stExon; 5′U | | | |
| | | | TR | | | |
| 1901 | cg17820591 | ENO3; ENO3 | TSS1500; TSS1500 | Island | NA | II |
| 1902 | cg17847344 | EBF1 | Body | S_Shore | TRUE | II |
| 1903 | cg17862447 | KLHL29 | 5′UTR | | TRUE | I |
| 1904 | cg17891759 | | | Island | NA | I |
| 1905 | cg17897699 | | | | NA | II |
| 1906 | cg17922359 | | | Island | NA | I |
| 1907 | cg17933583 | | | S_Shore | NA | I |
| 1908 | cg17933722 | | | Island | TRUE | II |
| 1909 | cg17939059 | LOC728661; CDK11B; | Body; Body; Body; | S_Shore | NA | II |
| | | CDK11B; CDK11B; C | Body; Body; Body; 5 | | | |
| | | DK11B; CDK11B; CD | ′UTR | | | |
| | | K11B | | | | |
| 1910 | cg17941572 | | | Island | NA | I |
| 1911 | cg17980508 | IFI44L | TSS1500 | | NA | II |
| 1912 | cg17990771 | | | | TRUE | II |
| 1913 | cg18000886 | ARX | TSS200 | Island | NA | I |
| 1914 | cg18018027 | | | N_Shore | NA | I |
| 1915 | cg18022005 | | | N_Shelf | NA | II |
| 1916 | cg18032812 | | | S_Shore | NA | I |
| 1917 | cg18034737 | ZFP42 | TSS200 | Island | NA | II |
| 1918 | cg18044383 | | | Island | NA | I |
| 1919 | cg18062721 | VGLL4; VGLL4 | Body; Body | | TRUE | I |
| 1920 | cg18063733 | | | Island | TRUE | I |
| 1921 | cg18065318 | C4orf49; C4orf49 | 5′UTR; 1stExon | Island | NA | I |
| 1922 | cg18070676 | FLRT2 | 5′UTR | Island | NA | I |
| 1923 | cg18091469 | | | Island | TRUE | I |
| 1924 | cg18093372 | | | Island | NA | I |
| 1925 | cg18096722 | | | Island | NA | II |
| 1926 | cg18102233 | ARX | TSS200 | Island | NA | I |
| 1927 | cg18108623 | SLFN11; SLFN11; SLF | TSS1500; TSS1500 | Island | TRUE | II |
| | | N11; SLFN11 SLFN11 | ; TSS1500; TSS150 | | | |
| | | | 0; TSS1500 | | | |
| 1928 | cg18117600 | | | Island | NA | I |
| 1929 | cg18118147 | SYT3; SYT3; SYT3 | Body; Body; Body | Island | NA | I |
| 1930 | cg18118685 | GALNT13 | 5′UTR | Island | NA | I |
| 1931 | cg18127012 | | | | TRUE | II |
| 1932 | cg18128969 | NCAM2 | TSS1500 | Island | NA | II |
| 1933 | cg18130044 | | | N_Shore | NA | I |
| 1934 | cg18132916 | | | Island | TRUE | I |
| 1935 | cg18133357 | ADARB2 | Body | | NA | I |
| 1936 | cg18142856 | OR10A3 | TSS200 | | NA | II |
| 1937 | cg18147485 | KCNV1; KCNV1 | 5′UTR; 1stExon | Island | TRUE | I |
| 1938 | cg18177414 | | | Island | NA | I |
| 1939 | cg18186343 | MIR770; MEG3; MEG3 | TSS1500; Body; Bo | | NA | II |
| | | ; MEG3 | dy; Body | | | |
| 1940 | cg18257955 | SNORD115-15; | Body; Body; TSS15 | | NA | II |
| | | SNORD115-21; | 00 | | | |
| | | SNORD115-22 | | | | |
| 1941 | cg18297736 | NKX6-1 | Body | N_Shore | NA | II |
| 1942 | cg18327669 | SSTR1 | TSS1500 | N_Shore | NA | II |
| 1943 | cg18333041 | CESTN2 | Body | | TRUE | II |
| 1944 | cg18342462 | C11orf88; C11orf88 | 1stExon; 1stExon | Island | NA | I |
| 1945 | cg18345826 | SYCP2L | TSS200 | N_Shore | NA | II |
| 1946 | cg18347921 | HLA-J; NCRNA00171 | Body; Body | Island | NA | I |
| 1947 | cg18355562 | | | | TRUE | II |
| 1948 | cg18358894 | | | Island | TRUE | I |
| 1949 | cg18388552 | | | | NA | I |
| 1950 | cg18395636 | RAB38 | TSS200 | S_Shore | NA | I |
| 1951 | cg18397279 | PCDH11X | 5′UTR | Island | NA | I |
| 1952 | cg18397998 | C1orf86; LOC1001280 | Body; TSS1500; Bo | N_Shore | NA | I |
| | | 03; C1orf86 | dy | | | |
| 1953 | cg18401367 | | | Island | NA | I |
| 1954 | cg18405691 | OR4C15 | 1stExon | | NA | II |
| 1955 | cg18420599 | DTNA; DTNA; DTNA; | TSS200; 5′UTR; 5′U | | TRUE | II |
| | | DTNA | TR; 5′UTR | | | |
| 1956 | cg18423469 | DLGAP1; DLGAP1 | Body; Body | N_Shelf | NA | II |
| 1957 | cg18435674 | KRT222 | Body | | TRUE | II |
| 1958 | cg18437710 | KLK10; KLK10; KLK1 | 5′UTR; 5′UTR; 1stE | S_Shore | NA | I |
| | | 0; KLK10 | xon; 5′UTR | | | |
| 1959 | cg18447419 | SLC9A3 | Body | N_Shore | NA | I |
| 1960 | cg18460175 | ETV2 | TSS200 | N_Shore | NA | II |
| 1961 | cg18461584 | | | | TRUE | II |
| 1962 | cg18465515 | HIST1H2BI | TSS200 | N_Shore | NA | II |
| 1963 | cg18470427 | SLFN12L | Body | | NA | I |
| 1964 | cg18472159 | | | Island | NA | II |
| 1965 | cg18479593 | | | Island | NA | II |
| 1966 | cg18492126 | NCAM2 | TSS1500 | Island | NA | II |
| 1967 | cg18503031 | MITF; MITF; MITF | Body; Body; TSS20 | | NA | II |
| | | | 0 | | | |
| 1968 | cg18512515 | | | N_Shore | NA | I |
| 1969 | cg18524788 | | | N_Shore | TRUE | I |
| 1970 | cg18527241 | ATXN7L1; ATXN7L1; | Body; 1stExon; 5′U | | NA | II |
| | | ATXN7L1 | TR | | | |
| 1971 | cg18530551 | | | Island | NA | I |
| 1972 | cg18536496 | GPRASP1; GPRASP1; | TSS1500; TSS1500 | Island | NA | I |
| | | GPRASP1 | ; TSS1500 | | | |
| 1973 | cg18595258 | TMEM138; CYBASC3 | TSS1500; 5′UTR; 5′ | N_Shore | NA | II |
| | | ; CYBASC3; TMEM13 | UTR; TSS1500 | | | |
| | | 8 | | | | |
| 1974 | cg18608369 | SLFN11; SLFN11; SLF | TSS200; TSS200; T | Island | TRUE | I |
| | | N11; SLFN11; SLFN11 | SS200; TSS200; TS | | | |
| | | | S200 | | | |
| 1975 | cg18631798 | ACOX3; ACOX3 | Body; Body | | NA | I |
| 1976 | cg18636509 | | | | TRUE | II |
| 1977 | cg18639180 | C14orf159; C14orf159; | | | | |
| | | C14orf159; C14orf159; | 1stExon; 1stExon; 5′ | | TRUE | II |
| | | C14orf159; C14orf159; | UTR; 1stExon; 5′UT | | | |
| | | C14orf159; C14orf159; | R; 5′UTR; TSS1500; | | | |
| | | C14orf159 | 1stExon; 5′UTR | | | |
| 1978 | cg18653451 | CCDC8 | TSS200 | S_Shore | NA | I |
| 1979 | cg18664869 | PCDHB15 | TSS200 | N_Shore | NA | I |
| 1980 | cg18673954 | INA | TSS1500 | N_Shore | NA | I |
| 1981 | cg18682423 | SLC46A3; SLC46A3 | TSS200; TSS200 | Island | NA | I |
| 1982 | cg18688293 | NMBR | 1stExon | N_Shore | NA | II |
| 1983 | cg18703601 | ROBO1 | TSS1500 | S_Shore | NA | I |
| 1984 | cg18709349 | PNMAL2 | TSS200 | Island | NA | I |
| 1985 | cg18724430 | | | N_Shore | TRUE | I |
| 1986 | cg18735015 | SH3PXD2A | Body | Island | TRUE | II |
| 1987 | cg18748888 | TNRC6C; TNRC6C | 3′UTR; 3′UTR | S_Shore | NA | I |
| 1988 | cg18755783 | SPG20; SPG20; SPG20; | 5′UTR; 5′UTR; 5′UT | Island | NA | II |
| | | SPG20 | R; 5′UTR | | | |
| 1989 | cg18766755 | SPRR2A | 5′UTR | | NA | II |
| 1990 | cg18769353 | PTN | TSS200 | | NA | II |
| 1991 | cg18782604 | SIM1 | Body | Island | TRUE | I |
| 1992 | cg18786593 | | | S_Shore | NA | I |
| 1993 | cg18786873 | ALX3 | Body | Island | TRUE | I |
| 1994 | cg18789887 | GAENTL2 | TSS200 | | TRUE | II |
| 1995 | cg18790597 | FAM184B | TSS200 | Island | TRUE | I |
| 1996 | cg18791205 | | | N_Shore | TRUE | II |
| 1997 | cg18794839 | KCNK2; KCNK2; KCN | TSS1500; TSS1500 | Island | NA | I |
| | | K2 | ; Body | | | |
| 1998 | cg18805027 | | | N_Shore | NA | I |
| 1999 | cg18816826 | | | | NA | II |
| 2000 | cg18844382 | EFS; EFS | TSS200; TSS200 | Island | NA | I |
| 2001 | cg18854419 | | | Island | NA | I |
| 2002 | cg18856581 | MDGA2 | TSS1500 | Island | NA | II |
| 2003 | cg18857062 | CRIP3 | Body | Island | TRUE | I |
| 2004 | cg18865445 | | | Island | TRUE | I |
| 2005 | cg18881873 | | | Island | TRUE | I |
| 2006 | cg18886245 | TFAP2B | Body | S_Shore | NA | II |
| 2007 | cg18891712 | FILIP1 | Body | | TRUE | II |
| 2008 | cg18894648 | LOC645323; LOC6453 | Body; TSS200 | N_Shore | TRUE | II |
| | | 23 | | | | |
| 2009 | cg18907180 | | | Island | NA | II |
| 2010 | cg18914211 | HLA-G | Body | Island | NA | I |
| 2011 | cg18922629 | SSBP1; FLJ40852 | Body; TSS1500 | S_Shore | NA | II |
| 2012 | cg18950778 | WT1; WT1; WT1; WT1 | Body; Body; Body; | Island | NA | II |
| | | | Body | | | |
| 2013 | cg18954504 | | | N_Shore | TRUE | I |
| 2014 | cg18956714 | OXGR1 | TSS200 | S_Shore | TRUE | II |
| 2015 | cg18989085 | OPCML | Body | | TRUE | II |
| 2016 | cg19022384 | | | Island | NA | I |
| 2017 | cg19030994 | PDCD6 | Body | S_Shore | NA | I |
| 2018 | cg19045293 | | | Island | NA | I |
| 2019 | cg19045644 | IGFBP7 | TSS1500 | Island | NA | I |
| 2020 | cg19102771 | CCDC140 | 5′UTR | N_Shore | NA | II |
| 2021 | cg19106893 | PTPN23 | Body | S_Shelf | NA | II |
| 2022 | cg19113530 | LOC100130691; AGPS | TSS1500; Body | S_Shore | NA | I |
| 2023 | cg19155518 | GRIK2; GRIK2; GRIK2 | Body; Body; Body | Island | TRUE | I |
| 2024 | cg19162768 | | | N_Shore | NA | II |
| 2025 | cg19170660 | HCG18; HCGI8 | Body; Body | N_Shore | NA | II |
| 2026 | cg19205850 | RAB7A | 5′UTR | S_Shore | NA | II |
| 2027 | cg19235071 | | | | TRUE | II |
| 2028 | cg19247350 | HIST1H2BH; HIST1H | TSS1500; TSS200 | N_Shore | NA | II |
| | | 3F | | | | |
| 2029 | cg19307500 | HMHA1 | Body | Island | NA | II |
| 2030 | cg19330592 | C14orf159; C14orf159; | 1stExon; 1stExon; 5′ | | NA | I |
| | | C14orf159; C14orf159; | UTR; 1stExon; 5′UT | | | |
| | | C14orf159; C14orf159; | R; 5′UTR; TSS1500; | | | |
| | | C14orf159; C14orf159; | 1stExon; 5′UTR | | | |
| | | C14orf159 | | | | |
| 2031 | cg19345277 | ZIM3 | 3′UTR | | NA | II |
| 2032 | cg19350360 | | | Island | NA | II |
| 2033 | cg19376851 | FLJ32063 | Body | Island | TRUE | II |
| 2034 | cg19378036 | HNF1B; HNF1B | TSS1500; TSS1500 | Island | NA | I |
| 2035 | cg19380001 | DMRTA2 | TSS1500 | Island | NA | I |
| 2036 | cg19382808 | LOC728661; CDK11B; | Body; Body; Body; | Island | NA | II |
| | | CDK11B; CDK11B; C | Body; Body; Body; 5 | | | |
| | | DK11B; CDK1B; CD | ′UTR | | | |
| | | K11B | | | | |
| 2037 | cg19391498 | GALM | TSS1500 | | NA | II |
| 2038 | cg19426944 | COL2A1; COL2A1 | Body; Body | N_Shore | TRUE | I |
| 2039 | cg19481013 | | | Island | NA | II |
| 2040 | cg19500479 | GDF7 | TSS1500 | Island | TRUE | II |
| 2041 | cg19513207 | | | Island | NA | II |
| 2042 | cg19513940 | DOK6 | Body | Island | NA | I |
| 2043 | cg19526130 | LOC729467 | Body | Island | NA | II |
| 2044 | cg19535896 | ZSWIM2; ZSWIM2 | 1stExon; 5′UTR | Island | TRUE | I |
| 2045 | cg19542346 | CDK4 | Body | N_Shore | NA | II |
| 2046 | cg19550524 | | | | NA | II |
| 2047 | cg19556343 | NCAM2 | TSS1500 | Island | NA | II |
| 2048 | cg19578835 | | | Island | NA | I |
| 2049 | cg19592336 | ZNF389 | TSS200 | | TRUE | I |
| 2050 | cg19595012 | PRDM8 | TSS200 | S_Shore | NA | I |
| 2051 | cg19595402 | SRCIN1 | Body | Island | TRUE | I |
| 2052 | cg19604907 | | | | TRUE | II |
| 2053 | cg19614643 | | | | TRUE | II |
| 2054 | cg19623012 | | | Island | TRUE | I |
| 2055 | cg19624849 | HFE2; HFE2; HFE2; HF | 5′UTR; 5′UTR; 5′UT | Island | TRUE | I |
| | | E2 | R; Body | | | |
| 2056 | cg19653212 | GUCA1A | Body | Island | NA | I |
| 2057 | cg19667811 | | | | NA | II |
| 2058 | cg19672997 | MDGA2 | TSS1500 | Island | NA | I |
| 2059 | cg19696441 | UBXN10 | TSS200 | Island | NA | I |
| 2060 | cg19697981 | NR2E1; NR2E1 | 5′UTR; 1stExon | Island | TRUE | II |
| 2061 | cg19715094 | TMEM25; TMEM25; T | Body; Body; Body; | S_Shore | NA | II |
| | | MEM25; TMEM25; TM | Body; Body; Body | | | |
| | | EM25; TMEM25 | | | | |
| 2062 | cg19726408 | SH3PXD2A | Body | Island | TRUE | I |
| 2063 | cg19731655 | MAP7D2; MAP7D2; M | TSS200; Body; Bod | Island | NA | I |
| | | AP7D2; MAP7D2 | y; Body | | | |
| 2064 | cg19743254 | OPCML; OPCML | Body; Body | | TRUE | II |
| 2065 | cg19777067 | | | Island | NA | I |
| 2066 | cg19793376 | BARHL1 | Body | Island | NA | II |
| 2067 | cg19807257 | | | Island | NA | I |
| 2068 | cg19810598 | | | Island | NA | I |
| 2069 | cg19811087 | | | Island | TRUE | I |
| 2070 | cg19811994 | RASSF1; RASSF1; RA | Body; Body; Body; 5 | N_Shore | NA | II |
| | | SSF1; RASSF1 | ′UTR | | | |
| 2071 | cg19844704 | EMX2; EMX20S; EM | Body; TSS200; Bod | Island | NA | II |
| | | X2 | y | | | |
| 2072 | cg19851715 | HIST1H2BI | TSS200 | N_Shore | NA | II |
| 2073 | cg19859323 | | | Island | NA | I |
| 2074 | cg19868631 | VSTM2A | TSS1500 | Island | NA | I |
| 2075 | cg19870698 | | | Island | NA | I |
| 2076 | cg19879075 | SPOCK2; SPOCK2 | Body; Body | Island | NA | I |
| 2077 | cg19887700 | | | Island | NA | II |
| 2078 | cg19889626 | | | Island | NA | I |
| 2079 | cg19929126 | TRIL | TSS200 | Island | NA | I |
| 2080 | cg19930575 | GABRE | 3′UTR | | NA | II |
| 2081 | cg19945554 | | | Island | NA | I |
| 2082 | cg19961043 | CHAD; ACSF2 | TSS1500; Body | Island | TRUE | I |
| 2083 | cg19961480 | CNR1; CNR1; CNR1; C | TSS1500; TSS1500 | Island | NA | I |
| | | NR1 | ; TSS1500; TSS150 | | | |
| | | | 0 | | | |
| 2084 | cg19971716 | QRFPR | 1stExon | Island | TRUE | I |
| 2085 | cg19974227 | RGS12; RGS12; RGS12 | Body; 5′UTR; 1stEx | N_Shelf | TRUE | II |
| | | ; RGS12 | on; Body | | | |
| 2086 | cg19988492 | DYRK1A; DYRK1A; D | Body; Body; Body; | | TRUE | II |
| | | YRK1A; DYRK1A; DY | Body; Body | | | |
| | | RK1A | | | | |
| 2087 | cg19989295 | REC8; REC8 | TSS200; TSS200 | Island | NA | I |
| 2088 | cg19991916 | PIGB | TSS1500 | N_Shore | NA | I |
| 2089 | cg19997502 | | | S_Shore | NA | I |
| 2090 | cg20000562 | SFTA3 | Body | S_Shore | TRUE | II |
| 2091 | cg20021336 | PARK2; PARK2; PAR | Body; Body; Body | | TRUE | II |
| | | K2 | | | | |
| 2092 | cg20041088 | | | Island | NA | II |
| 2093 | cg20055426 | | | Island | TRUE | I |
| 2094 | cg20059337 | AFF3; AFF3 | Body; Body | | TRUE | II |
| 2095 | cg20072171 | FEZF2 | Body | Island | NA | I |
| 2096 | cg20081453 | | | N_Shore | TRUE | I |
| 2097 | cg20101352 | CPVL; CPVL | TSS200; TSS200 | | TRUE | II |
| 2098 | cg20103519 | DOK6 | Body | Island | NA | II |
| 2099 | cg20107395 | DOK5; DOK5 | 1stExon; 5′UTR | Island | NA | I |
| 2100 | cg20120208 | TLX1 | 3′UTR | S_Shore | NA | I |
| 2101 | cg20170777 | COX10 | TSS1500 | N_Shore | NA | II |
| 2102 | cg20171396 | | | N_Shore | TRUE | II |
| 2103 | cg20202246 | | | Island | TRUE | I |
| 2104 | cg20229496 | | | N_Shore | TRUE | I |
| 2105 | cg20248458 | C2orf43 | 5′UTR | Island | NA | I |
| 2106 | cg20258486 | LOC154822 | TSS200 | S_Shore | NA | II |
| 2107 | cg20275132 | ZSCAN12; ZSCAN12 | TSS1500; TSS1500 | S_Shore | NA | II |
| 2108 | cg20282814 | SLC2A1 | Body | N_Shore | NA | II |
| 2109 | cg20289913 | NPFFR2 | 1stExon | Island | NA | I |
| 2110 | cg20293609 | CYR61 | Body | S_Shore | NA | I |
| 2111 | cg20295992 | | | Island | NA | II |
| 2112 | cg20297199 | BMP4; BMP4 | 5′UTR; 5′UTR | Island | NA | II |
| 2113 | cg20302533 | POU6F2; P0U6F2 | Body; Body | | NA | I |
| 2114 | cg20330472 | EYA4; EYA4; EYA4 | TSS1500; TSS1500 | N_Shore | NA | II |
| | | | ; TSS1500 | | | |
| 2115 | cg20337384 | | | Island | NA | II |
| 2116 | cg20340866 | | | N_Shore | NA | I |
| 2117 | cg20365336 | | | | TRUE | II |
| 2118 | cg20370678 | | | Island | NA | I |
| 2119 | cg20381767 | OXGR1 | TSS200 | S_Shore | TRUE | I |
| 2120 | cg20382028 | MCM3AP | Body | N_Shore | NA | II |
| 2121 | cg20394609 | EMB | Body | N_Shore | NA | II |
| 2122 | cg20395967 | MIR663 | TSS1500 | Island | NA | I |
| 2123 | cg20399509 | C21orf57 | 3′UTR | Island | NA | I |
| 2124 | cg20399616 | BCAT1 | Body | Island | TRUE | I |
| 2125 | cg20415053 | CATSPER4 | Body | | TRUE | I |
| 2126 | cg20418725 | CASP8; CASP8; CASP | 5′UTR; 5′UTR; 5′UT | | NA | II |
| | | 8; CASP8; CASP8 | R; TSS1500; 5′UTR | | | |
| 2127 | cg20426713 | SAMD13 | TSS1500 | N_Shore | NA | II |
| 2128 | cg20435267 | ZNF354B | Body | S_Shore | NA | II |
| 2129 | cg20435725 | | | N_Shore | NA | I |
| 2130 | cg20446252 | PASD1 | Body | | NA | II |
| 2131 | cg20449692 | CLDN11 | 1stExon | Island | TRUE | I |
| 2132 | cg20456954 | ARFGEF1 | Body | | TRUE | II |
| 2133 | cg20504856 | | | Island | NA | II |
| 2134 | cg20520804 | PFKP | Body | S_Shore | NA | I |
| 2135 | cg20555674 | ARL5C | Body | Island | NA | II |
| 2136 | cg20573931 | IL7 | Body | | NA | II |
| 2137 | cg20600210 | APCDD1L | TSS200 | Island | NA | I |
| 2138 | cg20615848 | C18orf34; C18orf34 | TSS1500; TSS1500 | S_Shore | NA | II |
| 2139 | cg20622056 | SLC7A3; SLC7A3 | TSS200; TSS200 | S_Shore | NA | II |
| 2140 | cg20623601 | EPAS1 | Body | Island | NA | II |
| 2141 | cg20640433 | LAMA2; LAMA2 | TSS200; TSS200 | | TRUE | II |
| 2142 | cg20647610 | WDR69 | TSS200 | Island | TRUE | I |
| 2143 | cg20658100 | KLF12 | 5′UTR | N_Shore | NA | I |
| 2144 | cg20668321 | GRIA1; GRIA1 | 3′UTR; 3′UTR | | NA | II |
| 2145 | cg20686203 | TM7SF2 | 3′UTR | N_Shore | NA | II |
| 2146 | cg20691205 | SPG20; SPG20; SPG20; | 5′UTR; 5′UTR; 5′UT | N_Shore | NA | II |
| | | SPG20 | R; 5′UTR | | | |
| 2147 | cg20711765 | | | | NA | II |
| 2148 | cg20711828 | LAMA2; LAMA2 | TSS200; TSS200 | | TRUE | I |
| 2149 | cg20743365 | LHX9; LHX9 | Body; Body | Island | NA | II |
| 2150 | cg20755170 | POU4F3 | 3′UTR | Island | NA | I |
| 2151 | cg20774846 | DPYS | TSS200 | S_Shore | NA | II |
| 2152 | cg20801007 | EFCAB4A | Body | Island | NA | I |
| 2153 | cg20808234 | | | Island | NA | II |
| 2154 | cg20842326 | PTPRN2; PTPRN2; PT | Body; Body; Body | Island | NA | II |
| | | PRN2 | | | | |
| 2155 | cg20852226 | MYO7A; MYO7A; MY | Body; Body; Body | Island | NA | I |
| | | O7A | | | | |
| 2156 | cg20883831 | KCNH2; KCNH2; KCN | TSS200; Body; Bod | Island | TRUE | I |
| | | H2 | y | | | |
| 2157 | cg20884605 | ALDH2 | Body | S_Shore | NA | II |
| 2158 | cg20885179 | VILL | Body | Island | NA | I |
| 2159 | cg20885782 | | | Island | NA | II |
| 2160 | cg20886017 | NEK11; NEK11 | Body; Body | | TRUE | II |
| 2161 | cg20891501 | SFTA3 | Body | S_Shore | NA | II |
| 2162 | cg20908789 | | | S_Shore | NA | II |
| 2163 | cg20916523 | VHL; VHL | Body; Body | S_Shore | NA | I |
| 2164 | cg20933109 | | | Island | NA | II |
| 2165 | cg20936013 | | | Island | TRUE | I |
| 2166 | cg20939084 | ZIC4; ZIC4; ZIC4; ZIC4 | Body; Body; Body; | Island | NA | II |
| | | ; ZIC4 | Body; Body | | | |
| 2167 | cg20950033 | KLHL33 | Body | | NA | I |
| 2168 | cg20981127 | NR2F6 | TSS1500 | S_Shore | NA | II |
| 2169 | cg21033440 | SIPA1; SIPA1 | Body; Body | Island | NA | I |
| 2170 | cg21051972 | | | Island | NA | II |
| 2171 | cg21052656 | AIP | TSS1500 | N_Shore | NA | II |
| 2172 | cg21064916 | | | N_Shore | NA | I |
| 2173 | cg21101743 | LOC646999 | Body | Island | TRUE | II |
| 2174 | cg21109265 | CRIP3 | TSS200 | Island | TRUE | II |
| 2175 | cg21117965 | SPEG | Body | | TRUE | I |
| 2176 | cg21120436 | NLGN1 | 5′UTR | | NA | II |
| 2177 | cg21144063 | | | | NA | II |
| 2178 | cg21146037 | TRIB1 | 3′UTR | | NA | II |
| 2179 | cg21146184 | | | Island | TRUE | I |
| 2180 | cg21158087 | | | Island | NA | I |
| 2181 | cg21179492 | MRPS2 | Body | S_Shore | NA | I |
| 2182 | cg21183256 | | | Island | TRUE | I |
| 2183 | cg21183523 | ZNF521 | Body | N_Shore | NA | II |
| 2184 | cg21196747 | | | | NA | II |
| 2185 | cg21205978 | APOL6 | TSS1500 | | NA | II |
| 2186 | cg21211480 | TMEM106A | TSS200 | Island | NA | I |
| 2187 | cg21253087 | SNAPC4 | Body | N_Shelf | NA | I |
| 2188 | cg21253459 | | | N_Shore | TRUE | II |
| 2189 | cg21263122 | SSTR1 | TSS200 | N_Shore | NA | II |
| 2190 | cg21268422 | BACE2; BACE2; BAC | Body; Body; Body | S_Shelf | NA | I |
| | | E2 | | | | |
| 2191 | cg21283066 | | | | TRUE | II |
| 2192 | cg21291134 | APCS | 3′UTR | | NA | II |
| 2193 | cg21293934 | ANKRD30B | TSS200 | Island | NA | I |
| 2194 | cg21350115 | CALCRL; CALCRL | 1stExon; 5′UTR | | TRUE | II |
| 2195 | cg21359793 | KRT4 | TSS1500 | | NA | II |
| 2196 | cg21359950 | CRADD | Body | | TRUE | II |
| 2197 | cg21362390 | SLITRK2; SLITRK2; S | 5′UTR; 5′UTR; 5′UT | Island | NA | I |
| | | LITRK2; SLITRK2; SL | R; 5′UTR; 5′UTR; 5′ | | | |
| | | ITRK2; SLITRK2; SLI | UTR; 5 | | | |
| | | TRK2; SLITRK2; SLIT | ′UTR; 5′UTR; 5′UT | | | |
| | | RK2 | R | | | |
| 2198 | cg21383487 | | | Island | TRUE | I |
| 2199 | cg21392341 | TBX15 | 5′UTR | Island | TRUE | I |
| 2200 | cg21397588 | ATP2B4; ATP2B4 | 5′UTR; 5′UTR | S_Shore | NA | II |
| 2201 | cg21461649 | CFTR | TSS200 | | NA | II |
| 2202 | cg21466913 | MAG11; MAG11; MAG | Body; Body; Body | | TRUE | II |
| | | 11 | | | | |
| 2203 | cg21476673 | HOXB8 | Body | Island | NA | I |
| 2204 | cg21485303 | | | Island | NA | II |
| 2205 | cg21507095 | TBX1; TBX1; TBX1 | Body; 3′UTR; Body | Island | TRUE | II |
| 2206 | cg21526205 | ST6GALNAC3; ST6G | Body; Body | Island | NA | I |
| | | ALNAC3 | | | | |
| 2207 | cg21532325 | WDR86 | TSS1500 | Island | NA | I |
| 2208 | cg21558752 | KRT4 | TSS1500 | | NA | II |
| 2209 | cg21560142 | BBC3; BBC3; BBC3; B | Body; Body; Body; | N_Shore | TRUE | I |
| | | BC3 | Body | | | |
| 2210 | cg21575509 | | | Island | NA | II |
| 2211 | cg21579726 | ABT1 | 3′UTR | S_Shore | NA | II |
| 2212 | cg21590238 | C12orf43 | TSS1500 | S_Shore | NA | II |
| 2213 | cg21595968 | | | Island | NA | I |
| 2214 | cg21620778 | WIPF1 | TSS1500 | S_Shore | NA | I |
| 2215 | cg21635854 | | | N_Shore | NA | I |
| 2216 | cg21642176 | RAD51AP2 | TSS200 | | NA | I |
| 2217 | cg21647584 | CT45A1 | 5′UTR | | NA | II |
| 2218 | cg21650962 | TFAP2B | Body | S_Shelf | TRUE | II |
| 2219 | cg21654383 | | | Island | NA | I |
| 2220 | cg21663122 | HIST1H2BH; HIST1H | TSS1500; TSS200 | N_Shore | NA | II |
| | | 3F | | | | |
| 2221 | cg21705897 | HMGCLL1; HMGCLL | TSS200; TSS200 | Island | TRUE | I |
| | | 1 | | | | |
| 2222 | cg21707187 | | | Island | TRUE | I |
| 2223 | cg21731375 | | | Island | NA | II |
| 2224 | cg21785034 | | | N_Shore | TRUE | II |
| 2225 | cg21797452 | LOC286467 | Body | Island | NA | II |
| 2226 | cg21817737 | NAALAD2 | TSS200 | | NA | II |
| 2227 | cg21819468 | PDPN; PDPN; PDPN; P | TSS1500; TSS1500 | Island | NA | I |
| | | DPN | ; 1stExon; 1stExon | | | |
| 2228 | cg21821214 | | | Island | NA | II |
| 2229 | cg21822905 | PEX10; PEX10 | Body; Body | S_Shore | NA | I |
| 2230 | cg21839338 | FAM18A | Body | Island | NA | II |
| 2231 | cg21843616 | | | S_Shore | NA | I |
| 2232 | cg21868134 | HPDL; HPDL | 1stExon; 5′UTR | Island | NA | I |
| 2233 | cg21881253 | MIR34B; BTG4; MIR3 | TSS200; TSS1500; | Island | NA | I |
| | | 4C | TSS1500 | | | |
| 2234 | cg21883598 | DUOX2 | Body | Island | NA | I |
| 2235 | cg21883802 | FBLN5; FBLN5 | 5′UTR; 1stExon | Island | NA | I |
| 2236 | cg21884231 | SLC7A14; SLC7A14 | 5′UTR; 1stExon | Island | NA | I |
| 2237 | cg21890239 | | | Island | NA | I |
| 2238 | cg21890726 | | | Island | TRUE | I |
| 2239 | cg21916358 | | | Island | NA | I |
| 2240 | cg21960265 | TNXB | Body | | TRUE | I |
| 2241 | cg21981270 | ALOX5 | Body | Island | NA | II |
| 2242 | cg21985631 | | | S_Shelf | TRUE | I |
| 2243 | cg22009464 | DTNA; DTNA; DTNA; | TSS200; 5′UTR; 5′U | | TRUE | II |
| | | DTNA | TR; 5′UTR | | | |
| 2244 | cg22012543 | | | S_Shore | NA | II |
| 2245 | cg22020954 | | | | NA | II |
| 2246 | cg22035501 | APC; APC; APC | TSS200; TSS200; 5′ | | NA | I |
| | | | UTR | | | |
| 2247 | cg22058855 | | | Island | TRUE | I |
| 2248 | cg22071194 | MOCS3; DPM1 | 1stExon; TSS1500 | Island | NA | II |
| 2249 | cg22091110 | | | Island | NA | II |
| 2250 | cg22135279 | | | | NA | II |
| 2251 | cg22152728 | | | Island | NA | I |
| 2252 | cg22160073 | TRIM34; TRIM34; TRI | 5′UTR; 5′UTR; TSS | | NA | II |
| | | M34; TRIM6- | 200; Body; 5′UTR | | | |
| | | TRIM34; TRIM34 | | | | |
| 2253 | cg22164577 | UBR3 | Body | S_Shore | NA | II |
| 2254 | cg22190437 | SPHKAP; SPHKAP | Body; Body | N_Shore | TRUE | I |
| 2255 | cg22198397 | SLC1A6 | Body | | NA | II |
| 2256 | cg22215784 | GPM6B | Body | N_Shelf | NA | II |
| 2257 | cg22237200 | ATP1B2 | Body | Island | TRUE | I |
| 2258 | cg22243996 | | | | NA | II |
| 2259 | cg22244418 | OGFOD1; NUDT21 | TSS1500; Body | N_Shore | NA | II |
| 2260 | cg22248052 | TFAP2B | Body | Island | NA | I |
| 2261 | cg22260952 | CHST11 | Body | S_Shore | NA | I |
| 2262 | cg22263031 | | | | NA | I |
| 2263 | cg22280258 | ALOX15 | TSS200 | S_Shore | NA | II |
| 2264 | cg22290648 | SYCN | 1stExon | Island | NA | I |
| 2265 | cg22294740 | LINGO3 | 5′UTR | Island | NA | I |
| 2266 | cg22303045 | | | N_Shore | NA | II |
| 2267 | cg22304522 | ADCY8 | Body | | TRUE | II |
| 2268 | cg22325530 | HVCN1; HVCN1 | TSS1500; 5′UTR | Island | TRUE | II |
| 2269 | cg22340072 | | | Island | NA | I |
| 2270 | cg22359606 | NEUROD1 | TSS200 | N_Shelf | TRUE | II |
| 2271 | cg22361859 | FBXO47; FBXO47 | 1stExon; 5′UTR | | NA | I |
| 2272 | cg22370379 | | | | TRUE | II |
| 2273 | cg22388634 | VSX1 | Body | Island | NA | I |
| 2274 | cg22389730 | FCRLB | Body | Island | NA | 1 |
| 2275 | cg22409854 | | | Island | NA | I |
| 2276 | cg22410478 | C6orf186 | Body | Island | TRUE | I |
| 2277 | cg22443762 | LHX9; LHX9 | Body; Body | Island | NA | II |
| 2278 | cg22445447 | RTF1 | Body | S_Shelf | NA | II |
| 2279 | cg22454011 | C18orf1; C18orf1; C18o | TSS1500; Body: Bo | | TRUE | II |
| | | rf1; C18orf1 | dy; TSS1500 | | | |
| 2280 | cg22459630 | LBXCOR1 | Body | Island | NA | I |
| 2281 | cg22462983 | EYA4; EYA4; EYA4 | TSS1500; TSS1500 | N_Shore | NA | II |
| | | | ; TSS1500 | | | |
| 2282 | cg22473637 | MCHR2; MCHR2 | TSS200; TSS200; | Island | TRUE | I |
| 2283 | cg22483095 | SBNO2; SBNO2 | Body; Body | Island | TRUE | I |
| 2284 | cg22489153 | | | Island | NA | II |
| 2285 | cg22491927 | CACNA1A; CACNA1 | 5′UTR; 1stExon: 1st | Island | NA | I |
| | | A; CACNA1A; CACN | Exon; 5′UTR | | | |
| | | A1A | | | | |
| 2286 | cg22496809 | TBX18 | Body | Island | NA | I |
| 2287 | cg22502502 | TRIM38 | TSS1500 | | TRUE | II |
| 2288 | cg22508969 | HMGCLL1; HMGCLL | TSS200; TSS200 | Island | TRU | I |
| | | 1 | | | | |
| 2289 | cg22512438 | TRH; TRH | 5′UTR; 1stExon | Island | NA | I |
| 2290 | cg22518208 | FLRT2 | 5′UTR | Island | NA | II |
| 2291 | cg22524657 | | | Island | TRUE | I |
| 2292 | cg22549870 | | | Island | NA | I |
| 2293 | cg22558291 | LOC646405 | Body | Island | NA | I |
| 2294 | cg22571664 | NPR3 | Body | Island | NA | II |
| 2295 | cg22605919 | C17orf104 | TSS1500 | Island | TRUE | I |
| 2296 | cg22612764 | FAM3B; FAM3B | TSS200; TSS200 | N_Shore | NA | I |
| 2297 | cg22618405 | | | S_Shore | TRUE | I |
| 2298 | cg22620221 | DPP6 | TSS200 | Island | NA | I |
| 2299 | cg22629987 | QPCT | TSS200 | Island | TRUE | II |
| 2300 | cg22630918 | C10orf2; C10orf2; C10o | 1stExon; 5′UTR; 1st | Island | NA | I |
| | | rf2; C10orf2 | Exon; 5′UTR | | | |
| 2301 | cg22639011 | KCNA4 | 5′UTR | Island | NA | II |
| 2302 | cg22658957 | | | Island | NA | I |
| 2303 | cg22694818 | LHX8 | 5′UTR | Island | TRUE | I |
| 2304 | cg22711792 | DMRTA2 | 3′UTR | Island | NA | I |
| 2305 | cg22724998 | | | N_Shore | TRUE | I |
| 2306 | cg22732749 | PCDHB15 | TSS1500 | N_Shore | NA | II |
| 2307 | cg22751696 | C3orf14 | TSS1500 | S_Shore | NA | II |
| 2308 | cg22759800 | | | Island | NA | I |
| 2309 | cg22771759 | | | Island | NA | II |
| 2310 | cg22784386 | SIM1 | Body | N_Shore | TRUE | II |
| 2311 | cg22795345 | ARHGEF7; ARHGEF7 | Body; Body; Body; | Island | NA | I |
| | | ; ARHGEF7; ARHGEF | Body; Body | | | |
| | | 7; ARHGEF7 | | | | |
| 2312 | cg22796353 | | | Island | NA | I |
| 2313 | cg22797169 | ILIRL2 | TSS200 | N_Shore | TRUE | II |
| 2314 | cg22798707 | ELAVL2; ELAVL2; EL | TSS1500; TSS1500 | Island | NA | II |
| | | AVL2 | ; 5′UTR | | | |
| 2315 | cg22809871 | FGF14 | Body | S_Shelf | NA | I |
| 2316 | cg22827864 | | | Island | NA | II |
| 2317 | cg22844669 | | | Island | NA | II |
| 2318 | cg22848598 | ADAM32 | TSS200 | Island | NA | I |
| 2319 | cg22865470 | | | Island | TRUE | I |
| 2320 | cg22870775 | | | S_Shore | NA | II |
| 2321 | cg22871668 | EYA4; EYA4; EYA4 | TSS200; TSS200; T | Island | NA | I |
| | | | SS200 | | | |
| 2322 | cg22881652 | | | S_Shore | NA | I |
| 2323 | cg22894896 | MIR193A | TSS200 | Island | NA | I |
| 2324 | cg22961278 | PRDM8 | 5′UTR | S_Shore | NA | I |
| 2325 | cg22968966 | | | Island | NA | II |
| 2326 | cg22971920 | NAA10 | Body | N_Shore | NA | II |
| 2327 | cg22976218 | NEUROD1 | 5′UTR | N_Shelf | NA | I |
| 2328 | cg22979093 | PTN; PTN | 5′UTR; 1stExon | | NA | II |
| 2329 | cg23003500 | VIPR1 | TSS1500 | Island | TRUE | I |
| 2330 | cg23017915 | WWTR1; WWTR1; W | Body; Body; Body | N_Shore | NA | II |
| | | WTR1 | | | | |
| 2331 | cg23026864 | PCDHA7; PCDHA12; P | Body; Body; Body; T | Island | NA | I |
| | | CDHA6; PCDHAC1; P | SS200; Body; Body; | | | |
| | | CDHA10; PCDHA4; P | Body; Body; Body; | | | |
| | | CDHA11; PCDHA8; P | Body; Body; Body; | | | |
| | | CDHA6; PCDHA1; P | Body; Body; Body; | | | |
| | | DHA2; PCDHA1; PCD | Body; TSS200; Bod | | | |
| | | HA9; PCDHA13; PCD | y | | | |
| | | HA5; PCDHA3; PCDH | | | | |
| | | AC1; PCDHA10 | | | | |
| 2332 | cg23028941 | EYA4; EYA4; EYA4 | Body; Body; Body | | TRUE | II |
| 2333 | cg23037133 | OXGR1 | TSS1500 | S_Shore | TRUE | II |
| 2334 | cg23049429 | ATP11A; ATP11A | Body; Body | S_Shore | NA | I |
| 2335 | cg23049458 | L1TD1; L1TD1 | 5′UTR; 5′UTR | Island | NA | I |
| 2336 | cg23056713 | NTM | Body | | TRUE | I |
| 2337 | cg23067317 | GAENTL2 | TSS200 | | TRUE | II |
| 2338 | cg23075337 | NXPH1 | Body | N_Shore | NA | II |
| 2339 | cg23085662 | | | Island | NA | II |
| 2340 | cg23090824 | PDE7B; PDE7B | 1stExon; 5′UTR | | NA | II |
| 2341 | cg23095615 | FBXO30 | TSS1500 | Island | NA | II |
| 2342 | cg23097402 | DMRTA2 | 3′UTR | Island | NA | I |
| 2343 | cg23113963 | SCNN1B | TSS1500 | Island | NA | I |
| 2344 | cg23118086 | RASL12 | Body | Island | NA | I |
| 2345 | cg23125200 | GJB6; GJB6; GJB6; GJB | TSS200; TSS200; T | Island | TRUE | I |
| | | 6 | SS200; TSS1500 | | | |
| 2346 | cg23141355 | | | Island | NA | I |
| 2347 | cg23158731 | COL14A1 | 5′UTR | Island | NA | I |
| 2348 | cg23161999 | | | N_Shore | TRUE | I |
| 2349 | cg23166590 | | | | NA | II |
| 2350 | cg23179456 | ADCY4 | TSS200 | Island | NA | I |
| 2351 | cg23180270 | RNF113A; NDUFA1 | TSS1500; Body | S_Shore | NA | II |
| 2352 | cg23189410 | ZIC4; ZIC1 | TSS1500; TSS1500 | N_Shore | TRUE | I |
| 2353 | cg23191956 | FOXG1 | TSS1500 | Island | TRUE | I |
| 2354 | cg23205387 | | | Island | NA | I |
| 2355 | cg23207876 | CASP14 | TSS200 | | NA | II |
| 2356 | cg23217097 | | | N_Shore | NA | I |
| 2357 | cg23218559 | AMH | Body | Island | NA | I |
| 2358 | cg23233404 | SYCP2L | Body | Island | NA | II |
| 2359 | cg23239574 | | | S_Shore | NA | I |
| 2360 | cg23244790 | PCDHGA4; PCDHGA | Body; Body; Body; | Island | NA | I |
| | | 11; PCDHGA11; PCDH | Body; Body; Body; | | | |
| | | GA9; PCDHGA1; PCD | Body; 1stExon; Bod | | | |
| | | HGB1; PCDHGB6; PC | y; Body; Body; Body | | | |
| | | DHGA12; PCDHGB3; | ; Body; 1stExon; Bod | | | |
| | | PCDHGB7; PCDHGA6 | y; Body; Body; Body | | | |
| | | ; PCDHGA8; PCDHGA | ; Body; Body; Body | | | |
| | | 10; PCDHGA12; PCDH | | | | |
| | | GA5; PCDHGB4; PCD | | | | |
| | | HGA3; PCDHGA2; PC | | | | |
| | | DHGA7; PCDHGB2; P | | | | |
| | | CDHGB5 | | | | |
| 2361 | cg23296408 | C14orf159; C14orf159; | 1stExon; 1stExon; 5′ | | TRUE | II |
| | | C14orf159; C14orf159; | UTR; 1stExon; 5′UT | | | |
| | | C14orf159; C14orf159; | R; 5′UTR; TSS1500; | | | |
| | | C14orf159; C14orf159; | 1stExon; 5′UTR | | | |
| | | C14orf159 | | | | |
| 2362 | cg23299641 | KCNE1L | 1stExon | Island | TRUE | I |
| 2363 | cg23303246 | NTM | Body | | TRUE | I |
| 2364 | cg23317501 | UGT3A1 | Body | | TRUE | II |
| 2365 | cg23318812 | | | Island | TRUE | I |
| 2366 | cg23328194 | ADRA1A; ADRA1A; A | Body; Body; Body | | NA | II |
| | | DRA1A; ADRA1A | Body | | | |
| 2367 | cg23338503 | DOCK2 | Body | Island | NA | I |
| 2368 | cg23363971 | IKZF4; IKZF4 | 1stExon; 5′UTR | | TRU | I |
| 2369 | cg23367107 | | | Island | NA | I |
| 2370 | cg23367608 | FAM162B | 1stExon | Island | NA | II |
| 2371 | cg23371754 | ANK2; ANK2; ANK2 | Body; Body; Body | | TRUE | II |
| 2372 | cg23398076 | MEIS1 | Body | S_Shelf | NA | I |
| 2373 | cg23424003 | EMID2 | TSS200 | Island | NA | I |
| 2374 | cg23424273 | HTR1B | TSS200 | Island | NA | I |
| 2375 | cg23438893 | | | N_Shore | TRUE | II |
| 2376 | cg23440636 | | | Island | NA | II |
| 2377 | cg23449696 | ZIC1 | TSS1500 | N_Shore | NA | I |
| 2378 | cg23468045 | | | | TRUE | II |
| 2379 | cg23473396 | | | Island | TRUE | II |
| 2380 | cg23477967 | TFP1; TFP1; TFP1; TFP1 | 1stExon; 5′UTR; 1st | | TRUE | II |
| | | | Exon; 5′UTR | | | |
| 2381 | cg23514417 | STK19; STK19; STK19 | TSS1500; TSS1500 | N_Shore | NA | II |
| | | ; DOM3Z | ; TSS1500; Body | | | |
| 2382 | cg23517208 | | | S_Shore | NA | I |
| 2383 | cg23519637 | C7orf50; C7orf50; C7or | Body; Body; Body | S_Shore | TRUE | II |
| | | f50 | | | | |
| 2384 | cg23522194 | PLA2G4C; PLA2G4C; | Body; Body; Body | Island | NA | I |
| | | PLA2G4C | | | | |
| 2385 | cg23524195 | GFRA1; GFRA1 | TSS1500; TSS1500 | Island | NA | I |
| 2386 | cg23536473 | FERD3L | 1stExon | Island | TRUE | I |
| 2387 | cg23549902 | | | Island | NA | I |
| 2388 | cg23554497 | | | S_Shore | TRUE | II |
| 2389 | cg23609450 | WFDC10B; WFDC10B | Body; Body | | NA | II |
| 2390 | cg23610453 | | | Island | TRUE | I |
| 2391 | cg23614791 | ACSS3 | TSS200 | Island | NA | I |
| 2392 | cg23624008 | | | S_Shore | TRUE | I |
| 2393 | cg23626733 | PEX10; PEX10 | TSS1500; TSS1500 | N_Shore | NA | I |
| 2394 | cg23629166 | PEX10; PEX10 | TSS1500; TSS1500 | N_Shore | NA | I |
| 2395 | cg23637354 | | | | NA | II |
| 2396 | cg23642200 | NLGN1 | 5′UTR | | NA | II |
| 2397 | cg23656300 | | | | NA | II |
| 2398 | cg23658477 | | | Island | NA | I |
| 2399 | cg23660154 | | | Island | NA | I |
| 2400 | cg23699274 | LRRK2 | TSS1500 | N_Shore | NA | II |
| 2401 | cg23715830 | | | Island | NA | I |
| 2402 | cg23720064 | SIM1 | Body | N_Shore | TRUE | II |
| 2403 | cg23759189 | NTM | Body | | NA | II |
| 2404 | cg23771603 | MYO3A | 5′UTR | Island | NA | I |
| 2405 | cg23776012 | KRT222; KRT222 | 5′UTR; 1stExon | | NA | II |
| 2406 | cg23779047 | | | | NA | I |
| 2407 | cg23782616 | HMSD | TSS200 | | TRUE | I |
| 2408 | cg23792314 | BCAT1 | Body | Island | TRUE | II |
| 2409 | cg23807890 | WDR69; WDR69 | 1stExon; 5′UTR | Island | TRUE | I |
| 2410 | cg23809442 | FGF14; FGF14 | Body; TSS1500 | S_Shore | NA | I |
| 2411 | cg23829990 | UNC50 | Body | S_Shore | NA | II |
| 2412 | cg23855505 | CHST11 | Body | S_Shore | NA | I |
| 2413 | cg23875663 | PCSK2 | Body | S_Shelf | NA | II |
| 2414 | cg23881566 | SRRM4 | Body | | TRUE | II |
| 2415 | cg23882545 | CASP8; CASP8; CASP | 5′UTR; 5′UTR; 5′UT | | TRUE | II |
| | | 8; CASP8 ; CASP8 | R; 5′UTR; TSS200 | | | |
| 2416 | cg23913995 | | | Island | NA | I |
| 2417 | cg23915769 | LVRN | TSS1500 | N_Shore | TRUE | II |
| 2418 | cg23928468 | SLC5A6; SLC5A6 | 5′UTR; Body | N_Shore | NA | II |
| 2419 | cg23952663 | LHX8 | Body | Island | NA | I |
| 2420 | cg23965214 | FBXO47 | TSS1500 | | NA | II |
| 2421 | cg24001644 | MAP7D2; MAP7D2; M | Body; Body; Body; 1 | Island | NA | I |
| | | AP7D2; MAP7D2; MA | stExon; 5′UTR | | | |
| | | P7D2 | | | | |
| 2422 | cg24002167 | EDIL3 | Body | N_Shore | NA | II |
| 2423 | cg24008884 | ALX4 | TSS1500 | S_Shore | NA | I |
| 2424 | cg24014143 | KLHL1; KLHL1; ATX | 1stExon; 5′UTR; Bo | Island | TRUE | I |
| | | N8OS | dy | | | |
| 2425 | cg24027397 | SOX2OT | Body | S_Shore | TRUE | II |
| 2426 | cg24041078 | BTG4; MIR34C | TSS1500; TSS1500 | Island | NA | II |
| 2427 | cg24046411 | CYBA | TSS200 | S_Shore | NA | II |
| 2428 | cg24046888 | EPAS1 | Body | Island | NA | II |
| 2429 | cg24052239 | CACNA1F | Body | Island | NA | II |
| 2430 | cg24061208 | ANKRD30B; ANKRD | 1stExon; 5′UTR | S_Shore | NA | II |
| | | 30B | | | | |
| 2431 | cg24067982 | TCERG1L | Body | N_Shore | NA | II |
| 2432 | cg24074153 | MDGA2; MDGA2 | Body; Body | | TRUE | II |
| 2433 | cg24104241 | ACTA1 | Body | Island | TRUE | II |
| 2434 | cg24106636 | NHLH2; NHLH2 | 5′UTR; 5′UTR | Island | TRUE | I |
| 2435 | cg24119607 | CXXC5 | 5′UTR | N_Shore | TRUE | II |
| 2436 | cg24146349 | INTS1 | Body | N_Shore | NA | II |
| 2437 | cg24149658 | CAPN6 | TSS200 | | NA | II |
| 2438 | cg24150623 | MIRI25B1; LOC39995 | TSS1500; Body | | TRUE | I |
| | | 9 | | | | |
| 2439 | cg24161501 | TCTEX1D1 | 5′UTR | Island | TRUE | II |
| 2440 | cg24211504 | SIM1 | Body | Island | TRUE | I |
| 2441 | cg24221648 | | | Island | TRUE | I |
| 2442 | cg24233426 | | | | NA | II |
| 2443 | cg24252695 | | | Island | NA | II |
| 2444 | cg24258699 | MSC | TSS200 | S_Shore | NA | I |
| 2445 | cg24279418 | | | N_Shore | NA | I |
| 2446 | cg24281267 | TULP1 | Body | Island | TRUE | I |
| 2447 | cg24302235 | SLC2A2 | TSS1500 | Island | TRUE | II |
| 2448 | cg24306340 | CACHD1 | Body | S_Shore | NA | II |
| 2449 | cg24315973 | GSTCD; GSTCD | TSS200; 5′UTR | S_Shore | NA | II |
| 2450 | cg24326574 | UBL4A | Body | N_Shore | TRUE | II |
| 2451 | cg24327132 | PKM2; PKM2; PKM2 | 5′UTR; 5′UTR; 5′UT | N_Shore | NA | II |
| | | | R | | | |
| 2452 | cg24331301 | CDH23; CDH23 | TSS I500; TSS1500 | Island | NA | I |
| 2453 | cg24335070 | | | | TRUE | II |
| 2454 | cg24338491 | | | | TRUE | II |
| 2455 | cg24346905 | NTNG1; NTNG1; NTN | Body; Body; Body | Island | NA | I |
| | | G1 | | | | |
| 2456 | cg24350771 | | | | NA | II |
| 2457 | cg24383507 | KCNIP2; KCNIP2; KC | Body; Body; TSS15 | N_Shelf | TRUE | I |
| | | NIP2; KCNIP2; KCNIP | 00; Body; Body; Bod | | | |
| | | 2; KCNIP2; KCNIP2 | y; Body | | | |
| 2458 | cg24384051 | OR8B3 | 1stExon | | NA | II |
| 2459 | cg24396429 | SLC8A1 | TSS200 | | TRUE | II |
| 2460 | cg24401557 | FGF13; FGF13; FGF13; | Body; TSS200; Bod | Island | NA | I |
| | | FGF13; FGF13; FGF13 | y; Body; Body; Body | | | |
| 2461 | cg24410453 | C6orf142 | Body | | TRUE | II |
| 2462 | cg24415208 | CACNG8 | 1stExon | Island | NA | I |
| 2463 | cg24427271 | USP36 | 5′UTR | N_Shore | NA | II |
| 2464 | cg24439334 | PEX10; PEX10 | TSS1500; TSS1500 | N_Shore | NA | I |
| 2465 | cg24448259 | CPVL; CPVL; CPVL; C | 5′UTR; 1stExon; 1st | | TRUE | II |
| | | PVL | Exon; 5′UTR | | | |
| 2466 | cg24452260 | GRIA2; GRIA2; GRIA2 | Body; Body; Body | Island | NA | II |
| 2467 | cg24472375 | | | N_Shore | NA | II |
| 2468 | cg24472939 | | | S_Shelf | NA | II |
| 2469 | cg24484138 | C20orf112 | 5′UTR | Island | NA | II |
| 2470 | cg24504927 | | | | TRUE | I |
| 2471 | cg24507144 | FGF14 | Body | Island | NA | II |
| 2472 | cg24508426 | | | Island | NA | I |
| 2473 | cg24509810 | CTNNA2; CTNNA2 | Body; Body | Island | NA | II |
| 2474 | cg24512400 | KLK10; KLK10; KLK1 | 5′UTR; 5′UTR; 5′UT | Island | NA | I |
| | | 0 | R | | | |
| 2475 | cg24517686 | | | S_Shore | TRUE | II |
| 2476 | cg24525457 | C7orf51 | Body | Island | NA | I |
| 2477 | cg24564492 | | | Island | NA | II |
| 2478 | cg24569637 | AOX1 | TSS200 | Island | TRUE | I |
| 2479 | cg24600895 | NLGN1 | 5′UTR | | NA | II |
| 2480 | cg24601030 | DIXDC1; DIXDCI | TSS1500; Body | N_Shore | NA | II |
| 2481 | cg24603941 | MIA2; MIA2 | 5′UTR; 1stExon | | NA | II |
| 2482 | cg24622143 | RASA3 | Body | Island | NA | II |
| 2483 | cg24625136 | | | Island | NA | I |
| 2484 | cg24626659 | C10orf93 | Body | N_Shore | NA | I |
| 2485 | cg24664038 | | | | NA | II |
| 2486 | cg24671344 | PDPN; PDPN; PDPN; P | TSS1500; TSS1500 | Island | NA | I |
| | | DPN | ; Body; Body | | | |
| 2487 | cg24672833 | | | Island | NA | I |
| 2488 | cg24686074 | | | Island | NA | I |
| 2489 | cg24686610 | ZIC4; ZIC1 | TSS1500; TSS1500 | N _Shore | TRUE | I |
| 2490 | cg24690692 | | | | TRUE | II |
| 2491 | cg24693341 | PHOX2B | 3′UTR | N_Shore | NA | II |
| 2492 | cg24701267 | | | | TRUE | II |
| 2493 | cg24701575 | | | N_Shore | NA | II |
| 2494 | cg24710320 | MGC2889; HRASLS | TSS1500; TSS200 | Island | NA | I |
| 2495 | cg24719107 | POSTN; POSTN; POST | 5′UTR; 1stExon; 1st | | NA | II |
| | | N; POSTN; POSTN; PO | Exon; 1 stExon; 5′U | | | |
| | | STN; POSTN; POSTN | TR; 5′UTR; 1stExon | | | |
| | | | ; 5′UTR | | | |
| 2496 | cg24722073 | | | N_Shore | NA | I |
| 2497 | cg24725574 | HLA-J; NCRNA00171 | Body; Body | Island | TRUE | I |
| 2498 | cg24725576 | | | N_Shore | NA | II |
| 2499 | cg24727130 | ELFN1 | 5′UTR | | NA | II |
| 2500 | cg24727399 | QRFPR | TSS200 | Island | NA | I |
| 2501 | cg24728018 | CLVS2; CLVS2 | 5′UTR; 1stExon | S_Shore | NA | I |
| 2502 | cg24734254 | | | | NA | II |
| 2503 | cg24740026 | | | Island | NA | I |
| 2504 | cg24740531 | BCL2; BCL2 | Body; Body | Island | NA | I |
| 2505 | cg24771804 | ONECUT2 | Body | Island | TRUE | II |
| 2506 | cg24773418 | | | Island | NA | I |
| 2507 | cg24805875 | FAM162B | TSS200 | Island | NA | II |
| 2508 | cg24812837 | TLX1 | Body | Island | TRUE | I |
| 2509 | cg24833737 | | | S_Shore | NA | I |
| 2510 | cg24843380 | ZNF454 | TSS1500 | Island | NA | I |
| 2511 | cg24843474 | RGS7 | TSS200 | Island | NA | I |
| 2512 | cg24848035 | RGS22 | TSS200 | Island | TRUE | I |
| 2513 | cg24852713 | HMHA1 | Body | Island | NA | II |
| 2514 | cg24853313 | MIR892A | Body | | NA | II |
| 2515 | cg24874180 | DKK2 | Body | N_Shore | NA | II |
| 2516 | cg24881834 | ME1 | TSS1500 | Island | NA | II |
| 2517 | cg24884142 | TBX15; TBX15 | 1stExon; 5′UTR | Island | TRUE | I |
| 2518 | cg24884703 | | | Island | NA | I |
| 2519 | cg24888814 | COPZ1 | Body | | TRUE | II |
| 2520 | cg24892966 | | | | NA | I |
| 2521 | cg24915503 | PCDHA7; PCDHA12; P | Body; Body; Body; | N_Shelf | NA | II |
| | | CDHA6; PCDHA10; P | Body; Body; Body; | | | |
| | | CDHA4; PCDHA11; P | Body; Body; Body; | | | |
| | | CDHA8; PCDHA6; PC | Body; Body; Body; | | | |
| | | DHA1; PCDHA2; PCD | Body; Body; Body; | | | |
| | | HA1; PCDHA9; PCDH | Body; Body | | | |
| | | A13; PCDHA5; PCDH | | | | |
| | | AC1; PCDHA3; PCDH | | | | |
| | | A10 | | | | |
| 2522 | cg24924958 | NMBR | TSS200 | Island | NA | II |
| 2523 | cg24928391 | SOX17 | TSS200 | Island | NA | I |
| 2524 | cg24932585 | | | Island | NA | I |
| 2525 | cg24939845 | | | Island | NA | I |
| 2526 | cg24945701 | | | Island | NA | I |
| 2527 | cg24956533 | LOC145845 | Body | S_Shore | NA | II |
| 2528 | cg24974737 | | | Island | TRUE | I |
| 2529 | cg24999518 | NHLH2; NHLH2 | 5′UTR; 5′UTR | Island | TRUE | II |
| 2530 | cg25029264 | C15orf26 | TSS1500 | N_Shore | NA | II |
| 2531 | cg25037730 | | | Island | TRUE | II |
| 2532 | cg25060829 | ZSCAN12; ZSCAN12 | TSS200; TSS200 | Island | NA | I |
| 2533 | cg25072733 | C13orf36 | 5′UTR | S_Shore | NA | I |
| 2534 | cg25113237 | | | Island | NA | I |
| 2535 | cg25119415 | MNDA | TSS1500 | | NA | II |
| 2536 | cg25124739 | EPAS1 | Body | Island | NA | II |
| 2537 | cg25129124 | KBTBD5 | 1stExon | Island | NA | I |
| 2538 | cg25156485 | MSN | Body | Island | NA | I |
| 2539 | cg25178784 | C11orf2 | Body | Island | NA | I |
| 2540 | cg25203962 | | | Island | TRUE | II |
| 2541 | cg25219333 | ARFIP2; FXC1 | Body; TSS1500 | N_Shore | NA | II |
| 2542 | cg25228422 | | | Island | NA | I |
| 2543 | cg25230235 | POLR21; TBCB | Body; TSS1500 | Island | NA | II |
| 2544 | cg25236484 | | | Island | NA | I |
| 2545 | cg25243455 | PAX8; PAX8; PAX8; P | Body; Body; Body; | Island | NA | I |
| | | AX8; PAX8; LOC4408 | Body; Body; Body | | | |
| | | 39 | | | | |
| 2546 | cg25266629 | TLX1 | Body | Island | TRUE | I |
| 2547 | cg25271525 | | | | NA | II |
| 2548 | cg25285904 | | | Island | NA | I |
| 2549 | cg25308354 | | | Island | NA | I |
| 2550 | cg25340966 | TBX15 | TSS200 | Island | TRUE | I |
| 2551 | cg25363445 | ALX4 | Body | Island | TRUE | II |
| 2552 | cg25368103 | | | | TRUE | II |
| 2553 | cg25374649 | C18orf1; C18orf1; C18o | TSS1500; Body; Bo | | TRUE | II |
| | | rf1; C18orf1 | dy; TSS1500 | | | |
| 2554 | cg25375162 | | | | TRUE | II |
| 2555 | cg25375916 | SLC33A1 | Body | N_Shelf | TRUE | II |
| 2556 | cg25387636 | SLC47A1 | TSS1500 | Island | NA | I |
| 2557 | cg25407876 | DNAJC9 | Body | N_Shore | NA | II |
| 2558 | cg25408008 | MIER1; WDR78; MIER | 5′UTR; TSS1500; 5′ | S_Shore | NA | II |
| | | 1; MIER1; MIER1; MIE | UTR; 5′UTR; 5′UTR | | | |
| | | R1; MIER1; MIER1; W | ; Body; 5′UTR; Body | | | |
| | | DR78 ; MIER1 | ; TSS1500; 5′UTR | | | |
| 2559 | cg25416067 | SEPT9; SEPT9; SEPT9; | 5′UTR; 5′UTR; Bod | | TRUE | I |
| | | SEPT9 | y; 1stExon | | | |
| 2560 | cg25456959 | ASB12 | TSS1500 | | NA | II |
| 2561 | cg25469406 | | | | NA | II |
| 2562 | cg25489866 | FAM81A | TSS1500 | N_Shore | NA | I |
| 2563 | cg25500080 | PCDHAC2; PCDHAC2 | 1stExon; TSS200; B | | | |
| | | ; PCDHA7; PCDHA12; | ody; Body; Body; Bo | | | |
| | | PCDHA6; PCDHA10; P | dy; Body; Body; Bod | | | |
| | | CDHA4; PCDHA11; P | y; Body; Body; Body | | | |
| | | CDHA8; PCDHA6; PC | ; Body; Body; Body; | | | |
| | | DHA1; PCDHA2; PCD | Body; Body; Body; 5 | | | |
| | | HA1; PCDHA9; PCDH | ′UTR; | | | |
| | | A13; PCDHA5; PCDH | | | | |
| | | AC1; PCDHA3; PCDH | | | | |
| | | AC2; PCDHA10 | | | | |
| Body | Island | NA | I | 5 | 140346199 | TGGGGAGT |
| | | | | | | CAGCGAGG |
| | | | | | | ACGGTGGG |
| | | | | | | GCCAGGAG |
| | | | | | | CCCTTGGG |
| | | | | | | AGGGCCTA |
| | | | | | | CG |
| 2564 | cg25510164 | GBA3; GBA3 | Body; Body | | NA | II |
| 2565 | cg25510609 | AKAP13; AKAP13; AK | Body; Body; Body | | TRUE | I |
| | | AP13 | | | | |
| 2566 | cg25514273 | KLHL1; ATXN8OS | 1stExon; Body | Island | TRUE | I |
| 2567 | cg25520146 | HPYR1 | TSS1500 | | NA | II |
| 2568 | cg25528646 | GABRE | TSS200 | Island | NA | I |
| 2569 | cg25556905 | ADCY4 | TSS200 | Island | NA | I |
| 2570 | cg25565383 | RAD51AP2 | TSS200 | | NA | II |
| 2571 | cg25566285 | PTPRN2; PTPRN2; PT | Body; Body; Body | S_Shelf | NA | I |
| | | PRN2 | | | | |
| 2572 | cg25588787 | | | Island | TRUE | II |
| 2573 | cg25594119 | | | N_Shore | NA | I |
| 2574 | cg25597760 | | | Island | NA | I |
| 2575 | cg25599924 | ACOX3; ACOX3 | Body; Body | | NA | II |
| 2576 | cg25602651 | DCAF8L1 | TSS200 | | NA | II |
| 2577 | cg25608973 | C2orf43 | 5′UTR | N_Shore | NA | II |
| 2578 | cg25610702 | CRIP3 | TSS1500 | S_Shore | TRUE | I |
| 2579 | cg25645064 | | | | TRUE | I |
| 2580 | cg25648436 | ZSCAN1 | Body | Island | NA | I |
| 2581 | cg25662926 | ZFHX4; LOC10019237 | 5′UTR; Body | S_Shore | NA | I |
| | | 8 | | | | |
| 2582 | cg25663755 | VILL | Body | N_Shore | NA | II |
| 2583 | cg25690715 | SEPT9; SEPT9; SEPT9 | 5′UTR; TSS200; Bo | | TRUE | II |
| | | | dy | | | |
| 2584 | cg25691167 | FERD3L | 1stExon | Island | TRUE | I |
| 2585 | cg25705558 | ISLR2; LOC283731; IS | 5′UTR; TSS1500; T | | | |
| | | LR2; ISLR2; ISLR2; ISL | 551500; TSS1500; 1 | | | |
| | | R2 | stExon; TSS200 | Island | NA | II |
| 2586 | cg25708328 | | | N_Shelf | NA | II |
| 2587 | cg25726664 | GLI3 | TSS1500 | Island | NA | I |
| 2588 | cg25729795 | FLJ30058 | Body | Island | NA | II |
| 2589 | cg25731337 | MAGEA5 | TSS200 | | NA | II |
| 2590 | cg25734928 | | | N_Shelf | NA | II |
| 2591 | cg25741487 | | | Island | NA | I |
| 2592 | cg25755283 | TBX15 | 5′UTR | N_Shore | NA | II |
| 2593 | cg25760325 | TRIML1 | TSS1500 | | NA | II |
| 2594 | cg25763036 | | | N_Shelf | NA | I |
| 2595 | cg25773917 | GPR183; UBAC2; UBA | 3UTR; Body; Body; | | NA | II |
| | | C2; UBAC2 | Body | | | |
| 2596 | cg25775494 | NR2E1 | Body | N_Shore | TRUE | I |
| 2597 | cg25782847 | C11orf88; C11orf88 | TSS200; TSS200 | Island | NA | I |
| 2598 | cg25800765 | ALX1 | TSS1500 | N_Shore | NA | II |
| 2599 | cg25810857 | 9-Sep | Body | S_Shore | NA | II |
| 2600 | cg25830182 | NKX6-1 | Body | N_Shore | NA | II |
| 2601 | cg25834768 | CAMKK1; CAMKK1; | 5′UTR; 5′UTR; 5′UT | Island | TRUE | I |
| | | CAMKK1 | R | | | |
| 2602 | cg25849196 | | | N_Shore | TRUE | I |
| 2603 | cg25859468 | MEOX2; MEOX2 | 1stExon; 5′UTR | | NA | I |
| 2604 | cg25872852 | RPLP2; SNORA52 | Body; TSS200 | S_Shore | NA | II |
| 2605 | cg25884711 | NPY; NPY | 5′UTR; 1stExon | Island | NA | I |
| 2606 | cg25887010 | | | Island | NA | II |
| 2607 | cg25896644 | | | Island | TRUE | I |
| 2608 | cg25913520 | | | N_Shore | TRUE | I |
| 2609 | cg25923056 | EGEN2; EGEN2; EGE | 1stExon; 5′UTR; 5′U | N_Shore | NA | I |
| | | N2 | TR | | | |
| 2610 | cg25928819 | | | N_Shore | NA | I |
| 2611 | cg25947619 | AKAP13; AKAP13; AK | Body; Body; Body | | TRUE | I |
| | | AP13 | | | | |
| 2612 | cg25951981 | GABRA4 | TSS200 | Island | NA | I |
| 2613 | cg25961816 | | | | TRUE | II |
| 2614 | cg25971727 | ACSS3 | TSS1500 | N_Shore | NA | I |
| 2615 | cg25977026 | KDM1A; KDM1A | Body; Body | S_Shore | NA | II |
| 2616 | cg25996119 | NKX2-1 | TSS1500 | Island | NA | I |
| 2617 | cg26000619 | AMR | Body | Island | NA | I |
| 2618 | cg26043257 | CD38 | 1stExon | Island | TRUE | II |
| 2619 | cg26043322 | OR10J5 | TSS1500 | | NA | II |
| 2620 | cg26053697 | COL4A2 | Body | S_Shore | NA | I |
| 2621 | cg26104752 | TBX15 | 5′UTR | S_Shore | NA | II |
| 2622 | cg26127662 | HOXA3; HOXA3; HO | 5′UTR; TSS1500; 5′ | N_Shore | NA | II |
| | | XA3 | UTR | | | |
| 2623 | cg26132320 | MMP9 | Body | Island | NA | I |
| 2624 | cg26151163 | ILKAP | 3′UTR | | NA | II |
| 2625 | cg26158950 | FLJ22536 | Body | S_Shore | NA | II |
| 2626 | cg26165269 | | | Island | NA | II |
| 2627 | cg26221992 | | | Island | NA | II |
| 2628 | cg26222247 | ANPEP | 5′UTR | Island | NA | II |
| 2629 | cg26232247 | RIN2 | Body | | TRUE | I |
| 2630 | cg26237681 | SYCP2L; SYCP2L | 1stExon; 5′UTR | Island | NA | II |
| 2631 | cg26238975 | MIR134; MIR668; MIR | TSS1500; TSS1500 | | NA | I |
| | | 382; MIR485 | ; Body; TSS1500 | | | |
| 2632 | cg26246880 | PYHIN1; PYHIN1; PY | TSS1500; TSS1500 | | NA | II |
| | | HIN1; PYHIN1 | ; TSS1500; TSS150 | | | |
| | | | 0 | | | |
| 2633 | cg26251098 | NTM | Body | | TRUE | II |
| 2634 | cg26253438 | ATP2B4; ATP2B4 | 5′UTR; 5′UTR | Island | TRUE | I |
| 2635 | cg26261502 | | | N_Shore | NA | II |
| 2636 | cg26262482 | CACHD1 | Body | S_Shore | NA | II |
| 2637 | cg26263234 | IRRC3B | 5′UTR | Island | NA | I |
| 2638 | cg26272220 | ACSS3; ACSS3 | 1stExon; 5′UTR | Island | NA | I |
| 2639 | cg26277556 | | | | TRUE | II |
| 2640 | cg26282792 | ZSCAN1 | Body | Island | NA | I |
| 2641 | cg26296488 | DRD5 | TSS200 | Island | NA | I |
| 2642 | cg26299756 | | | N_Shelf | NA | II |
| 2643 | cg26310256 | EGFL6; EGFL6 | Body; Body | S_Shore | TRUE | II |
| 2644 | cg26335602 | ZNF389 | 5′UTR | | TRUE | II |
| 2645 | cg26345888 | DAB1 | TSS200 | S_Shore | NA | I |
| 2646 | cg26354128 | KIAA1409 | 5′UTR | Island | NA | I |
| 2647 | cg26359388 | LOC286467 | Body | N_Shelf | NA | II |
| 2648 | cg26363759 | SPSB1I | 5′UTR | | TRUE | I |
| 2649 | cg26383972 | HTR2C | TSS1500 | N_Shore | NA | I |
| 2650 | cg26385743 | IMP4 | Body | S_Shore | NA | II |
| 2651 | cg26394257 | ADAM32 | TSS200 | N_Shore | NA | II |
| 2652 | cg26396492 | RIN2 | Body | | TRUE | II |
| 2653 | cg26398467 | | | | TRUE | I |
| 2654 | cg26398921 | | | Island | NA | II |
| 2655 | cg26404669 | CCDC65 | TSS200 | | TRUE | I |
| 2656 | cg26419656 | KLF12 | 5′UTR | N_Shore | NA | I |
| 2657 | cg26420013 | NSUN2; SRD5A1 | Body; TSS1500 | N_Shore | NA | II |
| 2658 | cg26432539 | SPATA8 | 1stExon | | NA | II |
| 2659 | cg26434983 | | | Island | NA | I |
| 2660 | cg26444528 | PCDH17 | 1stExon | Island | NA | I |
| 2661 | cg26447413 | GAS1 | 1stExon | Island | NA | II |
| 2662 | cg26449787 | EYA4; EYA4; EYA4 | TSS1500; TSS1500 | Island | NA | II |
| | | | ; TSS1500 | | | |
| 2663 | cg26459372 | ADCY1 | TSS1500 | Island | TRUE | I |
| 2664 | cg26465602 | | | Island | TRUE | I |
| 2665 | cg26466587 | MCHR2; MCHR2 | TSS200; TSS200 | Island | TRUE | I |
| 2666 | cg26499055 | SIM1 | Body | Island | NA | I |
| 2667 | cg26503653 | DOCK2 | TSS1500 | N_Shore | NA | II |
| 2668 | cg26517171 | TLX3 | TSS1500 | Island | NA | I |
| 2669 | cg26530061 | | | S_Shore | NA | II |
| 2670 | cg26530341 | TNFRSF10A | TSS1500 | S_Shore | NA | II |
| 2671 | cg26530706 | DTNA; DTNA; DTNA; | TSS200; 5′UTR; 5′U | | TRUE | II |
| | | DTNA | TR; 5′UTR | | | |
| 2672 | cg26560222 | DMRTA2; DMRTA2 | 1stExon; 5′UTR | Island | NA | I |
| 2673 | cg26560414 | CMTM3; CMTM3; CM | 5′UTR; 5′UTR; 5′UT | Island | NA | I |
| | | TM3; CMTM3; CMTM | R; 1stExon; TSS200 | | | |
| | | 3 | | | | |
| 2674 | cg26561986 | USP44; USP44 | 5′UTR; 5′UTR | N_Shelf | NA | II |
| 2675 | cg26567423 | FLJ12825 | Body | Island | NA | I |
| 2676 | cg26573704 | ADAMTS12 | TSS200 | Island | NA | II |
| 2677 | cg26583474 | RPS7 | Body | S_Shore | NA | II |
| 2678 | cg26596964 | | | Island | NA | I |
| 2679 | cg26597539 | PTGER4 | 5′UTR | Island | NA | II |
| 2680 | cg26603632 | | | N_Shore | NA | I |
| 2681 | cg26605683 | STC2 | Body | N_Shelf | TRUE | I |
| 2682 | cg26615127 | DNAJC6 | Body | Island | TRUE | I |
| 2683 | cg26618965 | | | Island | TRUE | I |
| 2684 | cg26623444 | | | Island | TRUE | I |
| 2685 | cg26625629 | C2orf43 | TSS200 | Island | NA | I |
| 2686 | cg26628435 | BEX1; BEX1 | 5′UTR; 1stExon | Island | NA | I |
| 2687 | cg26631984 | | | N_Shore | NA | I |
| 2688 | cg26650638 | | | Island | NA | I |
| 2689 | cg26677406 | SLCO3A1; SLCO3A1 | Body; Body | | TRUE | II |
| 2690 | cg26678970 | | | S_Shore | NA | I |
| 2691 | cg26679855 | | | N_Shelf | NA | II |
| 2692 | cg26702958 | FLJ41350 | Body | Island | TRUE | I |
| 2693 | cg26708220 | MEIS2; MEIS2; MEIS2 | Body; Body; Body; | Island | NA | I |
| | | ; MEIS2; MEIS2; MEIS | Body Body Body; | | | |
| | | 2; MEIS2 | Body | | | |
| 2694 | cg26717763 | | | S_Shore | NA | I |
| 2695 | cg26751645 | RBPJL | Body | Island | NA | I |
| 2696 | cg26755141 | SIM1 | Body | N_Shore | TRUE | II |
| 2697 | cg26764761 | JPH3 | Body | S_Shelf | NA | I |
| 2698 | cg26767897 | XDH | Body | | NA | II |
| 2699 | cg26769720 | | | Island | NA | I |
| 2700 | cg26772854 | | | Island | TRUE | II |
| 2701 | cg26796095 | SAFB | Body | S_Shore | NA | II |
| 2702 | cg26799474 | CASP8; CASP8; CASP | 5′UTR; 5′UTR; 5′UT | | NA | II |
| | | 8; CASP8 | R; 5′UTR | | | |
| 2703 | cg26814276 | GALNTL6 | TSS1500 | Island | NA | I |
| 2704 | cg26822986 | ANKRD29 | Body | Island | NA | II |
| 2705 | cg26827394 | UBE2I; UBE2I; UBE2I; | 5′UTR; 5′UTR; 5′UT | N_Shore | TRUE | I |
| | | UBE2I | R; 5′UTR | | | |
| 2706 | cg26835675 | SHISA9 | Body | | TRUE | II |
| 2707 | cg26836696 | | | N_Shore | NA | I |
| 2708 | cg26837708 | YBX1 | Body | S_Shore | NA | II |
| 2709 | cg26848718 | WT1; WT1; WT1; WT1 | Body; Body; Body; | Island | NA | I |
| | | | Body | | | |
| 2710 | cg26855689 | HGS | 3′UTR | N_Shore | NA | I |
| 2711 | cg26870192 | SLC35D3 | TSS1500 | N_Shore | NA | I |
| 2712 | cg26870638 | ZXDC; ZXDC | Body; Body | N_Shore | TRUE | II |
| 2713 | cg26874872 | | | | TRUE | II |
| 2714 | cg26891410 | TAL1 | TSS1500 | N_Shore | NA | II |
| 2715 | cg26896572 | C3orf14 | TSS1500 | Island | NA | II |
| 2716 | cg26917140 | | | N_Shore | TRUE | II |
| 2717 | cg26926076 | LAMA2; LAMA2 | Body; Body | | TRUE | I |
| 2718 | cg26926612 | | | Island | NA | I |
| 2719 | cg26929261 | ARL10 | TSS1500 | N_Shore | NA | I |
| 2720 | cg26937500 | CARD11; CARD11 | 1stExon; 5′UTR | | TRUE | 1 |
| 2721 | cg26954695 | MZF1; MGC2752; LOC | TSS1500; TSS1500 | N_Shore | NA | I |
| | | 100131691; MZF1 | ; Body; TSS1500 | | | |
| 2722 | cg26954968 | SIM1 | Body | Island | TRUE | I |
| 2723 | cg26966124 | SIM1 | TSS1500 | Island | TRUE | II |
| 2724 | cg26979473 | PTN; PTN | 5′UTR; 1stExon | | NA | II |
| 2725 | cg26983177 | | | Island | TRUE | I |
| 2726 | cg26992010 | | | N_Shore | TRUE | II |
| 2727 | cg26993I98 | | | S_Shore | NA | I |
| 2728 | cg26996175 | | | Island | NA | II |
| 2729 | cg27014380 | | | Island | NA | I |
| 2730 | cg27032232 | DPP6; DPP6 | 1stExon; 5′UTR | Island | NA | I |
| 2731 | cg27036111 | SLC6A5 | TSS1500 | S_Shore | NA | II |
| 2732 | cg27039467 | HN1L | Body | S_Shore | NA | II |
| 2733 | cg27039662 | | | Island | TRUE | I |
| 2734 | cg27062369 | DDIT4L | 5′UTR | Island | NA | I |
| 2735 | cg27071152 | LOC646999 | Body | Island | TRUE | I |
| 2736 | cg27080287 | | | Island | NA | I |
| 2737 | cg27088150 | | | N_Shore | NA | I |
| 2738 | cg27096043 | ATP11A; ATP11A | Body; Body | S_Shore | TRUE | II |
| 2739 | cg27109600 | FLJ32063 | TSS1500 | N_Shore | NA | I |
| 2740 | cg27112666 | | | N_Shore | NA | I |
| 2741 | cg27116888 | SALL3 | Body | | TRUE | I |
| 2742 | cg27120816 | CDH8 | TSS200 | Island | NA | I |
| 2743 | cg27127608 | EBF1 | Body | | TRUE | II |
| 2744 | cg27134730 | | | | TRUE | II |
| 2745 | cg27137084 | TMEM132C | Body | | TRUE | II |
| 2746 | cg27138281 | ME1 | TSS200 | Island | NA | I |
| 2747 | cg27140058 | BANK1; BANK1 | Body; Body | S_Shore | NA | I |
| 2748 | cg27153155 | SIM1 | Body | N_Shore | NA | II |
| 2749 | cg27198013 | RAT1 | 3′UTR | Island | TRUE | I |
| 2750 | cg27203895 | NTM | Body | | TRUE | II |
| 2751 | cg27225220 | LHX9 | Body | S_Shore | NA | II |
| 2752 | cg27228712 | | | Island | NA | II |
| 2753 | cg27252467 | LOC348021 | Body | | NA | II |
| 2754 | cg27252696 | SIM1 | TSS1500 | Island | NA | I |
| 2755 | cg27262412 | TBX15 | 5′UTR | Island | TRUE | II |
| 2756 | cg27268556 | | | N_Shore | NA | II |
| 2757 | cg27272235 | | | Island | TRUE | II |
| 2758 | cg27292588 | PTCH2 | 3′UTR | | TRUE | II |
| 2759 | cg27295832 | ANKRD34C | TSS1500 | Island | TRUE | II |
| 2760 | cg27297576 | | | N_Shore | NA | I |
| 2761 | cg27303421 | | | | TRUE | II |
| 2762 | cg27350602 | | | Island | NA | I |
| 2763 | cg27376707 | A2BP1; A2BP1 | 5′UTR; 5′UTR | Island | NA | I |
| 2764 | cg27401846 | | | N_Shore | TRUE | II |
| 2765 | cg27404023 | | | Island | TRUE | I |
| 2766 | cg27406678 | NKX6-1 | Body | N_Shore | NA | II |
| 2767 | cg27418402 | PSMD6 | Body | N_Shore | NA | II |
| 2768 | cg27422141 | PCDH9; PCDH9 | 5′UTR; 5′UTR | N_Shore | NA | II |
| 2769 | cg27424370 | | | N_Shore | NA | II |
| 2770 | cg27425314 | DHDDS; HMGN2; DH | 3′UTR; TSS1500; 3′ | N_Shore | TRUE | I |
| | | DDS | UTR | | | |
| 2771 | cg27452341 | CLCN1 | Body | Island | TRUE | II |
| 2772 | cg27493010 | | | Island | NA | II |
| 2773 | cg27510539 | VIPR1 | TSS1500 | Island | NA | II |
| 2774 | cg27522060 | RASL12; RASL12 | 5′UTR; 1stExon | Island | NA | I |
| 2775 | cg27527108 | | | Island | TRUE | I |
| 2776 | cg27528510 | EMID2 | TSS200 | Island | NA | I |
| 2777 | cg27531496 | | | | NA | II |
| 2778 | cg27541541 | PRL; PRL | TSS1500; 5′UTR | | NA | II |
| 2779 | cg27552679 | ISLR2; ISLR2; ISLR2; I | Body; Body; Body; | Island | NA | I |
| | | SLR2 | Body | | | |
| 2780 | cg27555582 | | | Island | NA | I |
| 2781 | cg27559661 | CPNE2 | TSS1500 | N_Shore | NA | I |
| 2782 | cg27564792 | FEZF2 | TSS1500 | S_Shore | NA | II |
| 2783 | cg27570984 | | | | NA | I |
| 2784 | cg27590397 | SSTR1; SSTR1 | 1stExon; 5′UTR | Island | NA | II |
| 2785 | cg27603915 | NELL1; NELL1 | Body; Body | S_Shore | NA | I |
| 2786 | cg27615938 | NCRNA00188; NCRN | Body; Body; Body; | S_Shore | NA | II |
| | | A00188; NCRNA0018 | Body; Body; Body; | | | |
| | | 8; NCRNA00188; NCR | Body; Body; Body; | | | |
| | | NA00188; NCRNA001 | Body; Body; Body; | | | |
| | | 88; NCRNA00188; NC | Body; Body; Body; | | | |
| | | RNA00188; NCRNA00 | Body; Body; Body; | | | |
| | | 188; NCRNA00188; NC | Body; Body; Body; | | | |
| | | RNA00188; NCRNA00 | Body; Body | | | |
| | | 188; NCRNA00188; | | | | |
| | | NCRNA00188; NCRN | | | | |
| | | A00188; NCRNA0018 | | | | |
| | | 8; NCRNA00188; NCR | | | | |
| | | NA00188; SNORD65; | | | | |
| | | NCRNA00188; NCRN | | | | |
| | | A00188; NCRNA0018 | | | | |
| | | 8; NCRNA00188 | | | | |
| 2787 | cg27635135 | | | | NA | II |
| 2788 | cg27638126 | MEOX2 | Body | | NA | II |
| 2789 | cg27663389 | | | Island | NA | I |
| 2789 | cg03456213 | C9orf3 | NM_032523 | Body | chr9: 9776664 | Island |
| | | | | | 9-97767955 | |
| 2789 | cg18315943 | | | | chr5: 3188099 | Island |
| | | | | | -3188375 | |
| 2789 | cg12737160 | COL4A6; COL4A6; CO | NM_ 033641; NM_ | 1stExon; Body; 5′U | | |
| | | L4A6 | 001847; NM_03364 | TR | | |
| | | | 1 | | | |
| 2789 | cg12098872 | | | | chr19: 477427 | Island |
| | | | | | 25-47743151 | |
| 2789 | cg18423852 | KCNH2; KCNH2; KCN | NM_172057; NM_ | TSS200; Body; Bod | chr7: 1506528 | Island |
| | | H2 | 172056; NM_00023 | y | 07- | |
| | | | 8 | | 150653080 | |
| 2789 | cg09771468 | BARHL1 | NM_020064 | Body | chr9: 1354619 | Island |
| | | | | | 34- | |
| | | | | | 135462909 | |
| 2789 | cg05733231 | | NR_024271 | Body | chr7: 4580818 | N_Shore |
| | | | | | 3-45808745 | |
| 2789 | cg04145287 | | | | chr1: 2655151 | Island |
| | | | | | 7-26552194 | |
| 2748 | cg27153155 | SIM1 | Body | N_Shore | NA | II |
| 2749 | cg27198013 | RAT1 | 3′UTR | Island | TRUE | I |
| 2750 | cg27203895 | NTM | Body | | TRUE | II |
| 2751 | cg27225220 | LHX9 | Body | S_Shore | NA | II |
| 2752 | cg27228712 | | | Island | NA | II |
| 2753 | cg27252467 | LOC348021 | Body | | NA | II |
| 2754 | cg27252696 | SIM1 | TSS1500 | Island | NA | I |
| 2755 | cg27262412 | TBX15 | 5′UTR | Island | TRUE | II |
| 2756 | cg27268556 | | | N_Shore | NA | II |
| 2757 | cg27272235 | | | Island | TRUE | II |
| 2758 | cg27292588 | PTCH2 | 3′UTR | | TRUE | II |
| 2759 | cg27295832 | ANKRD34C | TSS1500 | Island | TRUE | II |
| 2760 | cg27297576 | | | N_Shore | NA | I |
| 2761 | cg27303421 | | | | TRUE | II |
| 2762 | cg27350602 | | | Island | NA | I |
| 2763 | cg27376707 | A2BP1; A2BP1 | 5′UTR; 5′UTR | Island | NA | I |
| 2764 | cg27401846 | | | N_Shore | TRUE | II |
| 2765 | cg27404023 | | | Island | TRUE | I |
| 2766 | cg27406678 | NKX6-1 | Body | N_Shore | NA | II |
| 2767 | cg27418402 | PSMD6 | Body | N_Shore | NA | II |
| 2768 | cg27422141 | PCDH9; PCDH9 | 5′UTR; 5′UTR | N_Shore | NA | II |
| 2769 | cg27424370 | | | N_Shore | NA | II |
| 2770 | cg27425314 | DHDDS; H1GN2; DH | 3′UTR; TSS1500; 3′ | N_Shore | TRUE | I |
| | | DDS | UTR | | | |
| 2771 | cg27452341 | CLCN1 | Body | Island | TRUE | II |
| 2772 | cg27493010 | | | Island | NA | II |
| 2773 | cg27510539 | VIPR1 | TSS1500 | Island | NA | II |
| 2774 | cg27522060 | RASL12; RASL12 | 5′UTR; 1stExon | Island | NA | I |
| 2775 | cg27527108 | | | Island | TRUE | I |
| 2776 | cg27528510 | EMID2 | TSS200 | Island | NA | I |
| 2777 | cg27531496 | | | | NA | II |
| 2778 | cg27541541 | PRL; PRL | TSS1500; 5′UTR | | NA | II |
| 2779 | cg27552679 | ISLR2; ISLR2; ISLR2; I | Body; Body; Body; | Island | NA | I |
| | | SLR2 | Body | | | |
| 2780 | cg27555582 | | | Island | NA | I |
| 2781 | cg27559661 | CPNE2 | TSS1500 | N_Shore | NA | I |
| 2782 | cg27564792 | FEZF2 | TSS1500 | S_Shore | NA | II |
| 2783 | cg27570984 | | | | NA | I |
| 2784 | cg27590397 | SSTR1; SSTR1 | 1stExon; 5′UTR | Island | NA | II |
| 2785 | cg27603915 | NELL1; NELL1 | Body; Body | S_Shore | NA | I |
| 2786 | cg27615938 | NCRNA00188; NCRN | Body; Body; Body; | S_Shore | NA | II |
| | | A00188; NCRNA0018 | Body; Body; Body; | | | |
| | | 8; NCRNA00188; NCR | Body; Body; Body; | | | |
| | | NA00188; NCRNA001 | Body; Body; Body; | | | |
| | | 88; NCRNA00188; NC | Body; Body; Body; | | | |
| | | RNA00188; NCRNA00 | Body; Body; Body; | | | |
| | | 188; NCRNA00188; NC | Body; Body; Body; | | | |
| | | RNA00188; NCRNA00 | Body; Body | | | |
| | | 188; NCRNA00188; | | | | |
| | | NCRNA00188; NCRN | | | | |
| | | A00188; NCRNA0018 | | | | |
| | | 8; NCRNA00188; NCR | | | | |
| | | NA00188; SNORD65; | | | | |
| | | NCRNA00188; NCRN | | | | |
| | | A00188; NCRNA0018 | | | | |
| | | 8; NCRNA00188 | | | | |
| 2787 | cg27635135 | | | | NA | II |
| 2788 | cg27638126 | MEOX2 | Body | | NA | II |
| 2789 | cg27663389 | | | Island | NA | I |
| 2789 | cg03456213 | C9orf3 | NM_032823 | Body | chr9: 9776664 | Island |
| | | | | | 9-97767955 | |
| 2789 | cg18315943 | | | | chr5: 3188099 | Island |
| | | | | | -3188375 | |
| 2789 | cg12737160 | COL4A6; COL4A6; CO | NM_033641; NM | 1stExon; Body; 5′U | | |
| | | L4A6 | 001847; NM_03364 | TR | | |
| | | | 1 | | | |
| 2789 | cg12098872 | | | | chr19: 477427 | Island |
| | | | | | 25-47743151 | |
| 2789 | cg18423852 | KCNH2; KCNH2; KCN | NM_172057; | | | |
| | | H2 | | | | |
| NM_1 | TSS200; Bo | chr7: 150652807- | Island | | | GAGGGGCT |
| 72056 | dy; Body | 150653080 | | | | CAGGCTGC |
| ; NM_ | | | | | | GGGAGCGC |
| 00023 | | | | | | CGCTGCAG |
| 8 | | | | | | GAGCCTGG |
| | | | | | | GCGGGCTG |
| | | | | | | CG |
| 2789 | cg09771468 | BARHL1 | NM_020064 | Body | chr9: 1354619 | Island |
| | | | | | 34- | |
| | | | | | 135462909 | |
| 2789 | cg05733231 | | NR_024271 | Body | chr7: 4580818 | N_Shore |
| | | | | | 3-45808745 | |
| 2789 | cg04145287 | | | | chr1: 2655151 | Island |
| | | | | | 7-26552194 | |
| 2787 | cg27635135 | | | | NA | II |
| 2788 | cg27638126 | MEOX2 | Body | | NA | II |
| 2789 | cg27663389 | | | Island | NA | I |
| 2789 | cg03456213 | C9orf3 | NM_032823 | Body | chr9: 9776664 | Island |
| | | | | | 9-97767955 | |
| 2789 | cg18315943 | | | | chr5: 3188099 | Island |
| | | | | | -3188375 | |
| 2789 | cg12737160 | COL4A6; COL4A6; CO | NM_033641; NM_ | 1stExon; Body; 5′U | | |
| | | L4A6 | 001847; NM_03364 | TR | | |
| | | | 1 | | | |
| 2789 | cg12098872 | | | | chr19: 477427 | Island |
| | | | | | 25-47743151 | |
| 2789 | cg18423852 | KCNH2; KCNH2; KCN | NM_172057; NM_ | TSS200; Body; Bod | chr7: 1506528 | Island |
| | | H2 | 172056; NM_00023 | y | 07- | |
| | | | 8 | | 150653080 | |
| 2789 | cg09771468 | BARHL1 | NM_020064 | Body | chr9: 1354619 | Island |
| | | | | | 34- | |
| | | | | | 135462909 | |
| 2789 | cg05733231 | | NR_024271 | Body | chr7: 4580818 | N_Shore |
| | | | | | 3-45808745 | |
| 2789 | cg04145287 | | | | chr1: 2655151 | Island |
| | | | | | 7-26552194 | |

| | CHROMOSOME_ | MAP | |
|---|---|---|---|
| c | 36 | INFO | SourceSeq |
| 1 | 1 | 149719536 | CGGCCGCACCTCACTACCGACCTAAAGATGCGCCTTTGCTAGGCGGCAGC |
| 2 | 14 | 38067965 | CTTCCTATTTAGCAGGACCGCAGGGTTGGCAGACAGCAGGGCCTGGGCCG |
| 3 | 2 | 219848373 | GCCAACCTCGTTTTTGTGGTTTCTGTCTCTTGGTGAGATCTAGGGTAACG |
| 4 | 6 | 26271718 | GTAATTCACCAATCACATCGTTGGACTCTTCAGCCAATGGTTTTGTTGCG |
| 5 | 3 | 154146183 | CCTTTACTACTAAATGCTTCTCCTTTCAAAAGTCAGGGGCATGGGGATCG |
| 6 | 17 | 37123835 | GAGGGCACCATGGGAGCTGGCTCTCTTGCTATTCATAAAGCGGGGAGGCG |
| 7 | 2 | 228736253 | GGGGCGCAGAGCTGGGAGGTCTGGCTCCGCGGGCGGTTGGTGGCGACCCG |
| 8 | 15 | 53075505 | CACTGGCACACTGAGGTAATCGAAAAGATCAAACCTATCCTCGTCGATCG |
| 9 | 6 | 101840329 | GGCCCCTTCTTTATGCAGCCCGTTTGAAATGGTCTCTTGAGCCCAGTGCG |
| 10 | 6 | 100896392 | TGCCCACCCACCTGGAGTCCAGAAAGATGAGCTTCATGTCCAGGCTGGCG |
| 11 | 1 | 91185168 | TCTCATCGTCTCAGTGGCATTTTTCCTAGGGCTGTTCCGAGAGAACAGCG |
| 12 | 15 | 96888959 | TGTTAGAAAATTGTGCCATCTACCCGCTATGCTGAGTACTGTCACACTCG |
| 13 | 14 | 57261886 | CGGGTCCTAAAGGGGAGAGCTGAGCAGCCACTTCAGGGATTGCACTCCGA |
| 14 | X | 118107540 | CGTGCCCAGTTGTACCCTTCCCGCGCAGCGCTCAGGAGCGACAAACCTGG |
| 15 | X | 46306111 | CGCTGGCTCTGCAGCCCAGCTCAAAAGCCCATCCTCCAGGCCACTTCCCC |
| 16 | X | 43741826 | GAGTGGGGATTCTGGGGCGGGACTCTTTGAAGTCCTAGGTGACCTCTCCG |
| 17 | 1 | 50889225 | TGTGGCGCCTGCCATTGGCCAGGGGAGGCAGACGGCGCTCTGATTGGCCG |
| 18 | 5 | 172671561 | TTGGTGTGTGTGTGCAACACAACAATTTGTCAGCTGCTGTTCACAATGCG |
| 19 | 3 | 147111280 | CGGGCTGCAGGGGCCTAGCCCAACGCCGCCTCTTCACATTCACCCACCCA |
| 20 | 8 | 146023573 | CGGGCCAGTTTCTGGGATCTTGAAGTGTCTTTGCTGCTCCCTCTGATCAC |
| 21 | 5 | 32713290 | CGCCAGAATTCAGACACACAGGGGCATTTTTGGTGGAAACCACAGAAGCT |
| 22 | 13 | 97646341 | GTCAAATGTCTTCTCGCCCTGGGCTTTTTGTTCCCTAAAATGAAACTTCG |
| 23 | 6 | 108490371 | CGGGCCTGTGCCTCAGACTCCGCCACCCAGCCACGTTAAGGTGACTTGGG |
| 24 | 12 | 60091171 | CGCACGTGGGTGCACATGTGTATGTGAGTTTGAGATAGAAAAATGGGAAG |
| 25 | 8 | 142984613 | CGCATCAAGGTTCTCTGGCACTCAGCTCATCCAGCAGTTTGTCTCCTGCC |
| 26 | X | 139592612 | GAAGTGCCTGGAGGTCCACTGATTTTCCAGGCACCCTTTCCCGAGAAGCG |
| 27 | 11 | 20178496 | CGGCTTAGGTCCCACTGTACAGATGGGAAAACCGAGGCCTAGAGAGGGAG |
| 28 | 2 | 48982683 | CGCTGCCCCGGCCCCACGGCCGGTCTCACTCGACTGTGAGTACACAGCGC |
| 29 | 3 | 99594893 | CGCTCCACCAAGGGGCACTGCTGTCAGACAGCCAGGTAGCAAGTCTGTGG |
| 30 | 7 | 84671333 | GCAAACCTTACAGGAGTGGTTCTTACTAAAATTCACACGCAAAGTACACG |
| 31 | 11 | 6913644 | CCTTCTGGGACTCTCTGATGGGCCACACACCGAGCAGCTGCTATTTATCG |
| 32 | 1 | 1566374 | CGGCCCCCAGACCCTGCCTGGGACGCCCCCCAACTAACTCCCTGCTGGGA |
| 33 | 11 | 44332653 | TGGCAGGAAATCCCCTCGCCTGGGCGCAGACGTGGGGCGTGAACGGTGCG |
| 34 | 16 | 10276375 | ATCACAAGAGGGTCGCTGCGTGGGAAAGATCCACCTCCCTCGGGGAACCG |
| 35 | 10 | 13933247 | CGCCCTGTCCTGCACCCAAAGGAAAGGGCTCCCCTGGCACTCTCAGAAAA |
| 36 | 22 | 19278249 | TAAGAACAGGATTCAGACTCAAGTCTTTTTGACCACAAAGTAGTACATCG |
| 37 | 1 | 45286062 | CGGCGGGACAGGACCTTAACCCCTGGGACGAACACCAGCTCCGCAAAGGA |
| 38 | 5 | 72740460 | GCATTTAAGAGAATGGCAAGAAGGCATAGGGCGATCCAGGCCTCGCTCCG |
| 39 | 1 | 70035208 | CTGCAAACAGTAAGATTAGAGACCACTGGTAGCCAATCTAATAGTCCACG |
| 40 | 1 | 78511600 | CGCCCGCCCGCCCAGCCTCCCCCGGGATGGATCTGGAGAGAAAGAAAGTT |
| 41 | 16 | 56228114 | AAGCTCAAGAGAAGCCCCAAGCTAAATTACCCCTGGCTCAGAAGGGTGCG |
| 42 | 6 | 19691654 | CGGTGTAGGCTAATTTTGGAGGGAGTTCCACATTATTATCAATTTTTTTT |
| 43 | 2 | 96970381 | CGAGCAGCATTTTTGCTGGAATGCTTGGTGTAAGATCAGTGGCATTAAAA |
| 44 | X | 113818635 | CGGCTAGATTGCTAGCCTTGGCTGCTCCATTGGCCTGCCTTGCCCCTTAC |
| 45 | 6 | 37673597 | CGCTCTTGTTCTGGCCCCCACTCCTGCAGGCCACGCTGCGGATGGTTCAG |
| 46 | 15 | 24722944 | AGGGCGGTGAGATCTACAGGAGAACTCAGGGCGGATGTCTGCCGGGTGCG |
| 47 | 7 | 131812449 | CAAATTTCTCAGTCCCCAAGCAGGCCACGGGGACTGCAGCAAACAGAGCG |
| 48 | 12 | 106979451 | CGAACTGCTCCATGGAGCGCGCGTCTCTGCAAGCCAGGAGAAAAGACCCT |
| 49 | X | 101397146 | CGACGCCCCTTGCACTGAGCCCTCCATCCATTTTAGTGCTGGAAATGGGG |
| 50 | 15 | 60285691 | GGGGGGCGGGGTTCTTCGTATTAACGATTGCCCGTGGTCCTCAGCTTGCG |
| 51 | 6 | 100893665 | CGGACATTCTTTAAGAAGGTGCTTTGTATGTCTGAGACATCACAAGCATT |
| 52 | 2 | 219848333 | CGAGGTTGGCGACTGTTTTATCCAGAAGGAAACTATGCCCAACAGGAACG |
| 53 | 2 | 229046785 | AATCGACACAGGAAGCCAGAGGAGTGGCGCGCAACCAGTGGGGAGTGTCG |
| 54 | 18 | 67070170 | CGGAATGCTCAAGTCTGTCACCACACAGCTGTCAGCCAAGGGAGGTGCAG |
| 55 | 6 | 26240505 | CGCCCAGTAAGTTACGGAAAAGGCGGAGACAGAGGTTTCGTTCCCGCCCC |
| 56 | 11 | 7950225 | TTCAACCACACAGCTTTGATTTTGTCTTTCCATTTTCAGTAATGAAGTCG |
| 57 | 4 | 158144020 | GATCTAAAAGGATTGGTGTGGAGGCCAGAGGGCTCCGGTCCCCGCGCTCG |
| 58 | 15 | 45406486 | CGCTGCTATAAAAGGGGTCCCGCGCGACTTCCAAACTCAGCGCCAACCCG |
| 59 | 1 | 65731689 | CGCCGGGGCCTCCTTAAAACACAGCGCACTCGAAGCGCAGACTGCTGCGC |
| 60 | 11 | 86383430 | CGCAGCTCCTCACCCCGCCTGCTCCGCAGCGGGACCGCGTAGACCTCAGC |
| 61 | 1 | 26552116 | TTGCGTTGCCCACAAGCTTGTAGTCAGAAGAACTCGCCTGGGAGGGATCG |
| 62 | 10 | 27528611 | CGCTTAAGCTGGTAGACCCTATGAGTGTGTGGCATTAGGCATCCCCAGCC |
| 63 | 11 | 111783348 | CGGGCGGCGCTGGTCACACCCTCGTTGCTCTCACTCAGGGCCCCCACTCC |
| 64 | 2 | 242549864 | CTTGAGTGAGCCCTGTGACGCCTGCTAGGTGAGGTGGCAGCCAGTGAGCG |
| 65 | 16 | 22959868 | ATGCCTGGCCCAAAGCAGGGGTGCAGGCAGCTTAGGGATGACGAGTGTCG |
| 66 | X | 79278012 | GTCGAGGTGTCTCGCGGCTCCTAATGTGGCTGTCTGGCTGCGTCGGGACG |
| 67 | 13 | 76209541 | TAGGCTCCTTTGTGGAGACCTGATTTGAGCACTGGTGTGAAAAAGACCCG |
| 68 | 5 | 158524270 | CGCCCCTTCAGGCGACATAGACCCAGCTGACAACGGGACACACATGTCCC |
| 69 | 2 | 40677600 | CTTTCAGAATTCTCTCCTGGTGGCTCCCTCTTTCCCTTGGGAAATCTTCG |
| 70 | 8 | 70378994 | CGTCCTCAAGCACTCAGCACAGTGGTGTGTCAAGACGGGCTGCCCAATCC |
| 71 | 2 | 105459775 | GGGAGGACCCGGACGGGAGCAGGTTCCCTTCGCGCCTCTCTGGAGGGTCG |
| 72 | 18 | 31020754 | GGGAGTCGGGGGCTAAGGGCTCGAGCCAGGAGGCCCCGGAGCAGCAGCCG |
| 73 | 4 | 1647016 | CGCACGGAGCTTGCTCTGGCCATCTCTGTCCCTGCCTGTGCCATGGACAT |
| 74 | 6 | 21666778 | TCCACGAGGCGTTCGGACACTTTTTCCCGGCCTGACAGCAGAGAAAGTCG |
| 75 | 15 | 29034163 | CAGGGCGGTCGAAAGAGCCTTGGGGGCACATCTCGGGGTGCGGTGACCCG |
| 76 | 7 | 137028535 | GAGTATTCTGCTTTTAATAAGCTTCCCAATCAGCTCTCGAGTGCAAAGCG |
| 77 | 4 | 188916709 | CGGGGCCCCCAGGCACCCGGCCCGCACGCAAACCCTCAGCCCGGGGCCCC |
| 78 | 1 | 119542475 | TACAATGGCAAAATGCACTGTTGAGAACCCTTTGAAGTTGGGCAACCACG |
| 79 | 10 | 45914840 | GCTGGGGCGAAGATGAGTCCAGGGGTTGGGTTGAGAGCGCTGCGCAGCCG |
| 80 | X | 53030043 | GGGCCTGGGTGGAAACCAGGAGGACGGATTGCCGCCTGGGGCGCTCGCCG |
| 81 | 2 | 139537131 | GGCTTTCTGCCAGCTGGGGACACAAGATATAACTTTCATGCCCAGCTCCG |
| 82 | 6 | 74025019 | CGATGCCCACCAGCCTTACCGATTTAGGTAAAGGAGTAAATAAATTGGCA |
| 83 | X | 145077291 | CTTGAGGATGCCTGAGGTCCACGTGACCAACTAGGATCTTCTGGCATTCG |
| 84 | 10 | 102893925 | GGCTTCTTCTTCTTGGGGGGCGTCCGGTTCTGATAGGGGTGACCTATACG |
| 85 | 20 | 53092233 | GCCACTTTCGGGGAGCGCAGAGCGCGGCGCGGGGAGGCGAGGCTGGAGCG |
| 86 | 3 | 33701209 | CGCAGCTGCAGGGGCGCAGAGCACCCTCTATCCAAGCTGACAACCATCAG |
| 87 | 14 | 54357994 | CGGGATGTAAAAGTGTGGAAAACTTATTTCCCACGCAGAACCCTTGGTCC |
| 88 | 2 | 74404556 | CGAGTTGATATTTAATTAGTGAATGCACAGTTGCTTTGTGGACCTTAATA |
| 89 | 13 | 38066983 | CGTGGGAAGAAGCACCACATTCCTGTGGTCTTGGTTGACTGCTGAAATGC |
| 90 | 12 | 85674694 | GGCGGTAGGAACTGAAATCGATCACAGCTCGTTAGAGTACGTCACTGTCG |
| 91 | 14 | 52536022 | GGGGTAAAAGCTCCTGGCCAGGGCTGCCTGGAGCTGCCCCTTCCACTCCG |
| 92 | 2 | 88583117 | AACCAAGGAGACCAAAACCAGAATTCTCAAATCTAAAGTGGTTTAGGCCG |
| 93 | 12 | 89744471 | CGGCAAATTGGGGGTGACGTTCAAGATGTACTTGATGCCGAATTCCTCCA |
| 94 | 19 | 42007378 | CGCGTCAACTGAAATATTCAGGTTCTCACATTGGGACTGACTAGGCAAAC |
| 95 | 4 | 40588553 | CGCCTTACCCTGTTAACCAGAACAGCCATTGCATATTCCGTTCTGGGTTA |
| 96 | 1 | 229543254 | GTGGCAGATAAGACACGAGAGGGAGCTCTGGTCCCCGAGGGGCTGTCCCG |
| 97 | 3 | 156271246 | CGGCTACAGAAGCAGATTTTGAGGGTGGAATTAGAACACTTAAACACAGG |
| 98 | 16 | 49309445 | CGCAGCGTTTCCGGCACCCAGCCAGGCTTAAAGGTACTGTGAGGGGAACT |
| 99 | 7 | 15727441 | CGCCCGCCAAGCAAGTAGCAGCTTCAAAGCCCTGCCCTCGCTCTCGCATG |
| 100 | 7 | 127881269 | CCCTGAGGGGCGGGGCGGGAGCTGGCGCTAGAAATGCGCCGGGGCCTGCG |
| 101 | 21 | 46236351 | CGGCCTCTATGGAAGTCTGCACTCTGCTTGCTGTGGTCAGACTCCAGGGA |
| 102 | 7 | 100318718 | GGATTTAGCGCCCCGGCTATTGGCCAGGAGGTGGCTGGGTTCAAGGACCG |
| 103 | 7 | 87230344 | GAGGTGCAACGGAAGCCAGAACATTCCTCCTGGAAATTCAACCTGTTTCG |
| 104 | 8 | 56985462 | TGCCAGTGTCCAATAAAACCAGTCAGGTCTCACATATTATGTAGTTAACG |
| 105 | 1 | 47695321 | CGAACCCTCCAACTGGGATCGGTCTGGTTCAGTGTTTGTTTTCTAAGCAG |
| 106 | 19 | 39694617 | GGGTAGGTGCCGGCAGAGAACTTGTGCGTCTTGCCCGCCTTGCCTTGCCG |
| 107 | 4 | 57976915 | CTCAGGTGTCTCAGGCCCACGGAGGATTTAATAGATGAAACCCTGGATCG |
| 108 | 2 | 177029921 | CGCTTCGGTAAGGAATGAAAAGAAAAACACTTCCCAAGGCCCGGCACTTT |
| 109 | 2 | 3826621 | CGCAGGCAGCCGTGGACACGATCGGGTTTCCTGTGCCAGTCAATTACATG |
| 110 | 4 | 175132871 | CGGTCAAGACACAGGAACAGCCGTACTTTCCTGCGGGTAAGAGGCTTTTC |
| 111 | 7 | 27153663 | CGCACCCCCGGACCAGACGCTGCTCCGCGTGTAATATTCATAAGAAACAT |
| 112 | 2 | 223162049 | GCACGTAATTTCGCAGTCTTCTGTCCTATCCTCATGTTACAGCACCGACG |
| 113 | 14 | 36994016 | CGGGTCAAGCCCCACTAGAAGTTAACACCTGTGGTCTCCAAACTTGCCTA |
| 114 | 15 | 58357973 | CGTCCAATAAGAAGAGCCCCAGCTTGACACCTGCCTATATACAGGCGAGC |
| 115 | X | 13587976 | CCAGGAGAGCAGCAGCGGGAGCGCAAGGCTCCAGGGCAGAGGCATTCTCG |
| 116 | 13 | 36044860 | CGGGCCGGTTCGAACCGCTCCCTTAACCCTTAAGCAACCAGGACGCTCCC |
| 117 | 13 | 36919960 | CGGGAGGGCAAAAGACCACAGAGATCTGCGAAGCCAAAGTAAACAACGGG |
| 118 | 13 | 20716638 | CCAGGGGCGCAGCGGTGTGCGCATAGTAGGGTGGGAACCCGATGGCAACG |
| 119 | 17 | 40573764 | CGCCCCCACCCCCCGGCTCCGCCCGGGGCCACTTGTTGCTTTCTTTGCAG |
| 120 | 3 | 50373735 | CTGAAGCATCACTCACAACTCCACTTTCTTTACTTAAATAAGGATTTCCG |
| 121 | 1 | 40138052 | CGCAATCCCCAATCCGCTGCAGCCCACAAACCCAGAGAGCCAGAGAGCCC |
| 122 | 1 | 2838805 | CACACGGGGACGGAGCTGGGCTGGCAGCGGTGCCCAGGACAGAGTGGGCG |
| 123 | 1 | 110673082 | CGCGCACGATAGTCCCAACGCGGGCCTGTCTCACCTGGACTTCCTTCCCG |
| 124 | 7 | 53286921 | CTGTGAATTCCACCCACCGGGCATGGGAAAGTGCGGCATTCCCAGTCCCG |
| 125 | 5 | 4101132 | CTGTTGGCTTCTTATCACTGACAGCGGAGAAAGACTGATGGGCCTTGGCG |
| 126 | 6 | 49519250 | CGAGCTACTCATTAGAGTTTAATTTCCTCGAGCCAAGTCCCTGGAGACGG |
| 127 | 8 | 145114994 | CTGGAGTCCCACAGGAGCTCTTCAGCTGGTAGCCCTGGAAAAACTGGACG |
| 128 | 16 | 1575979 | CGGCTACCTGGAGGGCCTCCTGCCACCGGCCCGCAGCCTGGTAGAACTTG |
| 129 | 12 | 131622739 | GAGGCTGCACTCACGAGGTCCGAGTGGAAGGGCTTCGCGTTGGAGGTGCG |
| 130 | 12 | 57504948 | CGGGCACTTCCAGGCCCCATAGGTCTGTAGCTCTGTCCAGCGAGTTCAAG |
| 131 | 1 | 2336034 | GAGGATGACCCTGTATTCCTGGGTTAGTCAAGGTCAGCTTGCCAGAGACG |
| 132 | 1 | 761370 | GTAGAGAGTAACCAGCACCAGCTTCACAGGAATGGCATGACCTTGATACG |
| 133 | 12 | 22094461 | TTAAACTTCATTAGACACTGAAACTTGGAATATGATTCCATGATTCTCCG |
| 134 | 5 | 170739179 | CTATTTCACTTAACGCCGCTTTTATTTCGTTTTCGTTCCAATAATGCTCG |
| 135 | 14 | 77591726 | GTAATTTAATTTTCTCTCCCGAAAGCGTTGCTAATTAAATCCTTTCTCCG |
| 136 | 22 | 50690152 | CGCCTCCATGCAGCCACGACCACCGCCCTGAGCCGGCCGCAGCGCCACCT |
| 137 | 2 | 19558808 | CGCGGCTTGGGTTTCAACGCACTTTGGCGCCTGACAGAGTCGCATCTCCT |
| 138 | 17 | 45949828 | CGCCCGGCCTGTAGCTCCCCCCACGGCCCAGACGCAGGGAACTCCCCTCC |
| 139 | 5 | 160975792 | CGCTGACTCAGGAGACTTCGGAGTGTAGGCTGTCTAGTTCAGTGGTTAGG |
| 140 | 5 | 139284498 | AGGCCAGACCTGCAGGCATGAGAGGCACAGGCAACCCGCCTGTACGGCCG |
| 141 | 10 | 102896475 | AGGCCTCCTGCTGCAACTGCAGGAGGATGCGGTTCGCTTGCTGCCTCTCG |
| 142 | 11 | 89954679 | GCACTGCACCAATTCACACATGGATACAGACTTCCAACAAATGACTATCG |
| 143 | 2 | 21022844 | CCTGTCCACCTGGAAGGCTGCCCGCTCTCCCTGAGGGTCCTGGGAAGGCG |
| 144 | 11 | 61733046 | CGTCACAGCTACCTGTCTCTGGGGATCCCTAGTATAACACATTCAGTGTT |
| 145 | 4 | 25090298 | AAGTGGGGGGCAGGGGCTGTGCGCGGCTCTCTCCCCTAAAGCGAGGTTCG |
| 146 | 5 | 554886 | TTGGGGGCTGGGTCACGGGATGAACCGGGGGGTGGGTCACGGGATGAACG |
| 147 | 8 | 132053823 | CCAGGCGACTGGGAGAAATTTGGAGACCTGTCAGAGTGGTCAGACACCCG |
| 148 | 6 | 100911949 | CGGGTAAATCGGCAGCGAGACAGCCCCCTGCAAATAATGCTGGCATTCCC |
| 149 | 6 | 26172185 | TGGGCGTAACGGGGCGGCGGCGTAACGTGTGGCTCTGTGGTTAGGGCACG |
| 150 | 7 | 150949495 | CGCCCCAGTGAGGGTGATCGTATTCTCCTAAGGAGAGCTTTATTGCTGGG |
| 151 | 19 | 36132627 | CGGCCAGATTCCCCCACCCGCCCCCAGCGTCAGCTGACGTCACAATCGGC |
| 152 | 9 | 136224666 | CTGATGGGGGTCCACTGGGACTTACATTTACACAGAGCTCGCTGGTACCG |
| 153 | 17 | 33700757 | CGGCAGAGTTCGCGGGCTTAGCAGACCCGCCCATTGGCTCTTGCATCTCC |
| 154 | 3 | 62304554 | TCTTTATAAAGGGCCGCCTTGGGGCGCGAAAAGTTCGTGAGGTGTAGTCG |
| 155 | 6 | 100039476 | CGCAAGGGGGCTGAAGAGCCAAATGGGGAAACTGATGGAGCCTCACAAGT |
| 156 | 6 | 162068382 | CGCCTGTCGACGAGATGCTATTAACCAGTTTTTGCTCTGCCCCTGTCATG |
| 157 | 13 | 36919819 | TTTTACTCTTTACAATACATTCTTTCATGCTTCCTAGGGATTGAGCTGCG |
| 158 | 7 | 971767 | CGTGCAGACAACAGAATGGGGATGGAATCTCCTTGTGGCCCCCAGCTCAG |
| 159 | 2 | 231658979 | CGCTGGCGTGCAACACAGGCACTGACTCACACTTTGTCTAAACAAGCTTG |
| 160 | 2 | 162283705 | TGCGGTCGGTGGGGCGCAGGGCCGGGGAGCCAGGGGGGTGCCGGGGCTCG |
| 161 | 8 | 126450465 | CGTGGTTGCTCGACTGTTGTATCTGTGATACATTATCCGACTAAGGACTC |
| 162 | 6 | 100053305 | GAGCTTCTTTGAAGACCCTCCTCAGATAAACTGCTGAGTTGCAGGTGTCG |
| 163 | 1 | 119543336 | CGTGGCGGTCCCCTTGCTCATCTGACTCCGGAGGCAGGTTCCGCAGAAAG |
| 164 | 17 | 40120250 | GGACACAGTGGCCACGCTGCTAGAGTGCAAGACGCTCTTCATCTTGCGCG |
| 165 | 10 | 5734716 | CGCCCTTGTTGACTTTCAAAAGGCGCCGCCGGCCCCGCGCCCTACACGCT |
| 166 | 20 | 21489196 | TCCACGGGCTCCATGCTGGATTCCATATGGCCAGCTGGGCCGAGGGAGCG |
| 167 | 19 | 39755181 | CGCCCAGCCCTTGCGCCCCCCGGTACACTGGCCCCGCCGGGTTCCCACAC |
| 168 | 10 | 73847259 | CGCGCCCTTCTGCACGGGGACCCCAGCCCACCCCCGTACGGACACGCCTT |
| 169 | 14 | 74035742 | CGCAGGCCAGCAAGCTTCTGAAAAGCAAACCTAGGAAGTAGCTTTCCAAC |
| 170 | 4 | 2963264 | TCGTCCAGTTGACTCCAAGCTTGATAACGCGTGTAGGGGCCGTGAGGCCG |
| 171 | 20 | 25129184 | CGCCCCCAGGGCCCGGCGTCCTGGGCCTCTGACTCTGCCCGGTTTGCTAA |
| 172 | 17 | 32595801 | CGAGAGAATGAGTAGCCTGAGCAAAGTCATAGGGGTAAGATAAAACGACA |
| 173 | 1 | 64937528 | ACAGAAAAACTGCCCGCAGCTAGTAAGGAATCCTGATGATGTTACTCACG |
| 174 | 14 | 95235489 | CGTGGGCACGCTGTCGCGCACCGAGCTGCAGCTTCTCAACCAGCTGCACT |
| 175 | 2 | 38892901 | CGGTGGCTATCAGACGAAGAATGGGAAAATACAGATAGCAGACTTACAGT |
| 176 | 5 | 10333766 | AATGCGCCCTGACTCCCCCTGGCCGAGAGCCGGCTCAGAATGAAAGAGCG |
| 177 | 6 | 26271816 | TGGAAAATCTGATTGGATGAATCACTTCATTTTTGAATCCTCTCTTCTCG |
| 178 | 13 | 114109526 | CGGGATAAGGTTAGCTCGTTTATCCAACAAATATTTGCAGAGCCCCTACT |
| 179 | 11 | 31820572 | CGGACTAAGTGGGCCAGACTCTTGTCAGGGGAGGGATCACCAGGACCTCA |
| 180 | 4 | 74810017 | AGGCTACCAGGAGCAGGAGCAGCAGCGCCGCCCGCAGTAGCCGGGGAGCG |
| 181 | 11 | 50227199 | TAGATGAGGCCCGGCGGGTTCCTCCCCTCAGGCTAGGCGGGGTGGCAGCG |
| 182 | 6 | 101841008 | TCTGAGGGAATACCCGCAGCAGCGCTACCAGAGCGCATTTGCTCTATGCG |
| 183 | 16 | 73101264 | AACTTACAGTCTTCAGGGAAGGGCTTCCTGCCAACTGGAACAGTGGTGCG |
| 184 | 12 | 679900 | CGGCCTCCACCCGCCACAAGCGCACAGGGCTCTGCCCACTCCCCGCCCCT |
| 185 | 13 | 70682098 | ACCTGGGCGTGGGGACACCACCAGGCAGGAGCAGAGGCAGGACTGGGACG |
| 186 | 15 | 53097777 | AGGGCGCCGGCAGGCGGCTGAGGCAGAACGGTTGGGCTGCGGTGCCTCCG |
| 187 | 22 | 19754125 | CGGAGCCGCTGCACCACCACCCCTACAAATATCCGGCCGCCGCCTACGAC |
| 188 | 3 | 129346694 | CGGAAGTGACCAAGCGCGACTAGATCTAAAGCCTTCCCGACTTCCATTTT |
| 189 | 16 | 62070184 | CGGACACCCAGAGCCCAGCGCGCCCGCCCTGCGTCTCCGCACCAAGCCCT |
| 190 | 6 | 28603292 | CGCGGACTCCTTGTCCTTGCCGGTTCACCTCCCTGTGGAGGACCTCCTCT |
| 191 | X | 101395217 | CGAGCCAGGTCAGTGGGCACTGTTGCTAGGTCCTCTATCATGTTCCTGGG |
| 192 | 6 | 32223076 | TGAAATCTTGCGGTGTTGAGTAGGACCGCCAGGCCGGCAAGCAGGAGGCG |
| 193 | 7 | 137028546 | CGAGAGCTGATTGGGAAGCTTATTAAAAGCAGAATACTCCAGGGGCAGTC |
| 194 | 17 | 36728693 | ATTAGGTGCCTTCGGCGTGGGCCGGCCCCCACTCCAAAATCAGGCTGGCG |
| 195 | X | 70151160 | CGCCAGGCTTGCTAGTGAGGATGGGACGCAGTCTCAGGCCCCACGCAATC |
| 196 | 6 | 96463902 | CGCCGCGCGCGCTCTCTGCCCATCTCCGGCTTCGCTGAAGCTGCTACAGC |
| 197 | 7 | 96626788 | CGGTCATCAACTGAAATAGGCAAAACCAATAACGCACCTCGCTTTTCGCC |
| 198 | X | 106750146 | TAAGGTGTCTTCCTACAGACACCGCTGCTGCTACCTTGCTACCTTCAGCG |
| 199 | 12 | 131694034 | GGCGTCTTCTGCGCATTTGTTGAAGTTACAGAGCCCTCGTCATGATCCCG |
| 200 | 14 | 62583795 | TGGGAGGCTGGGGCTGGGCGAGACTCGCGGGCCCGGGTAGGTGAGCCTCG |
| 201 | 2 | 182545485 | CGTGGTTCCAGGCTCTTGGCTGGACCGGGAAGACCATATGGCGCATGCCG |
| 202 | 11 | 89954258 | CGGTGCTGTGGCATAATTACCGTGCTCGTTGACTTATAGTCTCATGATTT |
| 203 | 12 | 63026212 | CGCTCTTTGTAAATACAGAATAGGACAAAACGGAAAGAGTGATGTGCTGT |
| 204 | 6 | 133563764 | CGCCGCACAGCAACCCGAACTTTCGTTTGGTAGCACTTGAAAGAGTTTTC |
| 205 | 6 | 27637395 | AACCACAAACCTGGTAGTCCAAATGTGTTGGGCTGTGGCCAAGTTGAACG |
| 206 | 6 | 29974723 | CGCGCCCGCGTCTTCATCCTCAGACTCACGGCGTCACTGTCGACCCGCAC |
| 207 | 2 | 21022699 | CGCAGAGCCCCTGTTGCCCTGGGCCAGATGGCCCGTAGCAGCAAAAATAA |
| 208 | 12 | 34530799 | CGCCCCACAAGCAAAAGTAATTCCCAACCAAGCATGCGCAGTGCTCAGGG |
| 209 | 13 | 79234251 | CGCAGTATGACTTCAAGAAGCAGCCTCATGGTTAAGAGAAAGGGGTTCTG |
| 210 | 3 | 62304779 | CGCGGCCGTCAGGCAGTCAGGGCACCCACCCACTGTCCGACTCCACTCAC |
| 211 | 17 | 48050274 | CGCGTGGAGCGTTTCAACCCCCACGTTCACCGCGCCAGGTGATACAAGAC |
| 212 | 6 | 96463185 | CTGCCCCTTGGAGCTGTTGCCTGGGCAGGGGTGGCCTTGGTTGTTTACCG |
| 213 | 2 | 223169965 | AAGGTCGAAGAGTCGTTACCAAAACCTTTGAAGCCGCTTTTCGGCTCTCG |
| 214 | 7 | 1027033 | TCGTGGAGCACGGCGCTTGTGTAGGGCAGAGCCTGCTGGTCCTCCAGCCG |
| 215 | 8 | 93074929 | CGCCCAGCACCCTGCCTCCGGCGATGCCGCCCCAGCCCCTCTGATGGCCC |
| 216 | 6 | 19692217 | GAGCGCATACCAAAGGCAGACTCCTTTGCAGGCTCCACGAGCCCCATTCG |
| 217 | 2 | 177026301 | CGGTGGTCAGCCAGCTCCCTGCATTGTTCCGGTAGCGAGCTCCACACACA |
| 218 | 14 | 54430382 | CGCCCCACAGCTGGCTCCCGCGCTCTGCCCACCGCCACGCCACCTTCACC |
| 219 | 17 | 76101114 | CGCTGACCCACCGCAGCCCGCACTGTTTTGCGAACAGTTCGCAAGGACTG |
| 220 | 6 | 31938886 | CTGCCTCTCAGGGGTCCAGGCTGGCTGGCAGAGGCCATAGTGACGTGGCG |
| 221 | 2 | 97000811 | CGGTCCAGGTGGGGAAGAACTGATGCAGAAATGCTATGTATACATTAAAT |
| | | | (This probe can belong to Red or Green) |
| 222 | 6 | 100906383 | CGGCCACTGTCCCTGAAGGGCAAGCAGTCGTCTAAACCTTAGGTCCAGAT |
| 223 | X | 125687041 | CGCGTGTCTGATACTTGGGATCCTGGCTTGCGGGCCCTCCTAGACTCTGC |
| 224 | 6 | 129204339 | CGCCCTGTCCCCTGGCCTTGGCTTGTGAGCTGAGCGAGCAGCAGCTGCTG |
| 225 | 16 | 22959525 | CCGAGCTGGTGGTAAATTGCATCTCTCGACAGAGGTGGCTGAGGGAAGCG |
| 226 | 12 | 104852355 | CGGAACGTTCTACCTGTTGGGCTTCGCAGCCGAGCTGCTCACCCCCGGCC |
| 227 | 2 | 146044461 | CATTGCATCAAGCCTTGAGTGTGCATGATAAGCAGGATGTATGCCCTTCG |
| 228 | 8 | 122651486 | TCTATATATACTCAGGCCTCCAGTTGGGGGAAGCCCTTGGTGGCCAATCG |
| 229 | 15 | 37389951 | CAAGATTCTGGGGACTCAGTCACTCCAGGCTGTGGGGTCTGGAGTGGTCG |
| 230 | 2 | 185462493 | CGGCAAAGGCGCTGCTGCCTCAAGCTTGCTTTACATGCCTCTCTAGTTCC |
| 231 | 8 | 143916273 | CGGAGCCCCGCAGTGTGCGTGCCCGCCGCCTCCAGCCATCTGAGCCTCCC |
| 232 | 6 | 100896803 | AATGTCCTTGAACGTCATTAGCAGGTTTCAGGGACCTACTTCGCTATTCG |
| 233 | 8 | 60032607 | CGGACAGTTGCCCACTCACAGCGTGCAAAGGGGTCGCACTCCCTGTGGGT |
| 234 | 5 | 172751331 | TCTCCGACGACAGCTAGTTTCTATAGTAATGTGGGGTGCGGGGATTCCCG |
| 235 | 10 | 101282726 | TGGAAAGCCCTGTGACCTATTCCCAGGGACCCAGCAGTGGGCTCCCAGCG |
| 236 | X | 139592127 | CCCACAGAGTATTCAGGTGTTAATCACCAGCTCTCTTTTCCCTATGTGCG |
| 237 | 14 | 103740476 | CTGCTGGAGGGCCCAGCCCCTGGGGCCTCGGGGTCGTTCGGGTCCTGACG |
| 238 | 19 | 15162623 | GAGCAGGGCAAGTGACCCATGTGTTTGCCTCTTCTACCTAGGAGATGACG |
| 239 | 5 | 139228153 | GTGCCGCAGTAACGGCTGCTGCTCGGCCAGGCGGTAACTGATGGGCGCCG |
| 240 | 17 | 10632861 | TGGAAGCGAGTCCCATACCAGGGCTCACACCCTGGAAGGGGCTGGTTCCG |
| 241 | 6 | 117086479 | CGCCCTCAAGCCGACTCCCGGGCCGGCCCCTCGCTGCCCTCCCGCAACCT |
| 242 | 4 | 74734874 | CCTCGCCCTTCAGAGTAACTCCTGTGGACTCTGAGACTCTGGGATATTCG |
| 243 | X | 70151117 | CGCAATCCTTTTGCAAAGCCAAGAAGTGTGTGTGTTGTGTGTGTGTGTGT |
| 244 | 3 | 149374914 | GGAAGAAGATCCTGCCGGAGTCTTTCTTTAAGGAGCCTGATTCGGGCTCG |
| 245 | 17 | 20687681 | CGCGCCAGTCAACTCCGCGCACGTTGCCCCTGCTTGGCAGCCAATAAAAG |
| 246 | 11 | 122852454 | CGAGCAGGCTTGGAGACTCTGCCTGCGTCCTCCACTTCGTCCCGGCCCTT |
| 247 | 17 | 59529618 | CGCGGTGGAGGAGCCGGCGGCTCGCGTGGGGCTCGGGATCTTGGGGACCG |
| 248 | 14 | 57276527 | ATCCATCCAAACGACCCCAACCAAATCTGGGGGCCAAAACGCAAAGATCG |
| 249 | 2 | 223176167 | CGCCCGCAAAGACGCCGCATTCCAAATGCAGAGCAGATCGGCTGTAACAG |
| 250 | 10 | 83636325 | CGCCCGCCGGTAGGAAGGAAAGAAAGCAAAGCACATGCAAATTGACCGAC |
| 251 | 2 | 223160297 | GACGGCACGGTGTTTCGATCACAGACCGCGTCCTTGAGTAATTTGTCTCG |
| 252 | 6 | 108440118 | CTATTATCACTCACTACATTTCCCAGCTATGTACTGAAAAAATCAGCTCG |
| 253 | 14 | 56424872 | CGGAGGCTGTACAGTCATGCTAATGAGCTGCTGGACAATGGCAGTTTTTG |
| 254 | 2 | 201450690 | CGGAGAAAGCCTTGATTAAGATCCAACGTTCCTGAGCTCTTTGCCAGGAA |
| 255 | 8 | 126448033 | CGCCTGGGGTGGAGGGTATGAGGCAAAGCCCCAAATAGTATTCATAATGC |
| 256 | 20 | 9819697 | GAGGACTGTCCATGAGCGTACGCAGCTGCTCCGGGTTTTCGTGGCAGCCG |
| 257 | X | 106359455 | CGGCACTCTCGATGGAGTTCCATTTTGTTGCAGTGGTGCTTCAGCTTAGA |
| 258 | 7 | 19183732 | CGGACCACAGCTCCTTGTCCCAGGGCCACAGTGGTGTTCATTCAGTGCCT |
| 259 | 4 | 15779999 | CGGCCTCATCTTCGCCCAGCCAACCCCGCCTGGAGCCCTATGGCCAACTG |
| 260 | 4 | 72897565 | AGGATCTGGAGCCAAGTAATGGTGATACTGATGCTTCCTTTTCTTTGCCG |
| 261 | 6 | 26273199 | GGAGCCCTTCTTCGGGGCGGGAGCTGACTTAGCTGGTTCAGGCATGTTCG |
| 262 | 19 | 39523158 | GAGGGGCGTGGGGAGCTTGCGGCACAGCTAGGGCATCCCGGCGAGGAGCG |
| 263 | 7 | 134144055 | CGCCCCGCGTGGCAGAAAGGAGTTCCTCAGCTGCTGTATCTGGTGGGAAA |
| 264 | 15 | 89438611 | GGGGCTGTGTGCGGGCCGGGTTCCTCCCTTACCGCTTTGGGTGCCCTGCG |
| 265 | 13 | 29293325 | ATTCGGATTCAGAAATGCTTTGCAACTCCAGAGCTGTTGAGCCTGCGGCG |
| 266 | 14 | 57264095 | CTCATTCTGGTTGTTTTCCACCGCGTCTGCTCCCATTTGCGTGGGATGCG |
| 267 | X | 51487529 | AAATTGTGAAATGTTTCCTTTCTGTTTCAAAATAGGCACGACCCACTTCG |
| 268 | 2 | 85640943 | GGTCCTCTCTTAGGGGCGCGCCGTCTCCTGGCTTCCTGTAAGAAAAGGCG |
| 269 | 8 | 38585881 | CGCTGGCCCTGTCTGGAATTTAAAGCTCCGTCTGAATTCTGGTGCCTTTT |
| 270 | 3 | 130235850 | GTGACGGAAGCGGGACTGGAGACATTATGAAGGCTGTTACTGCTCCACCG |
| 271 | 10 | 21653473 | TTCTGACAAATCTCTTTGGAATCCTCACTTCATTCAAAGATTCAAGCTCG |
| 272 | 16 | 51183988 | GGAGGACACCTGGAATCCCTCCTTGCTCCTAAAGGTGTTTCACGGTACCG |
| 273 | 14 | 85996202 | CGGACCAGACCCAGCCCTCGGAGTCTATTTTTTGCCTCCAGTTACCGGAT |
| 274 | 12 | 103355958 | CGCCGCACAAGCCCCGGCTTTGTGTTGCTAAGCGATTAGAGCAGATGTAA |
| 275 | 6 | 84140923 | CGGATGATCTCCTGCCATGACTCAGCGCTTCTCGCAGGCTGCCCTGCTGG |
| 276 | 15 | 37084662 | CTGTTAGCCGTTCACCTCGTGCCCGCAGACTTCTCAAAAAATACAAATCG |
| 277 | 19 | 37893981 | AGTTAGATCTGCAGTTCACCTTAATTTTGGCAGCTCACTGTATCATAACG |
| 278 | 12 | 108237572 | CGGACCTAGTGAGCCAGGCAGAGCCTTTAGGCCCACGCCTGGGCACCACA |
| 279 | 16 | 10102278 | CCCACTTCTTATGCTCTACAAAACAACAGCAGTAGGGGAGTGACTGCACG |
| 280 | 10 | 124909028 | CGCCAGCTCTCCCTGGGACCAAGCGGTTGGCAAGCGCCTGCAGCCTTGGT |
| 281 | 7 | 152622368 | ACAGAAGGCATTTTGGGCTAAGGACGCTCACAACCCGGTTTCCTGTCCCG |
| 282 | 6 | 37673335 | TGGCGCCCCCTGTAGTTTATGCCACGCAGCGGTCGCGGGTGTGCTGAACG |
| 283 | 6 | 133562101 | TTCCGGGGGCTGCCCCACAGAAGCAGGTGGGGATCGACAGTGGCTCTCCG |
| 284 | 6 | 74024628 | CGAACGGTGAATCTGCTATGGTAGGGACGGCGCGGACCTTGGAGCCATCG |
| 285 | 14 | 42075554 | GATGCTGAGCTGAGTTGATTCCCAAATATAAATGTTCTTTTCAGAATCCG |
| 286 | 12 | 103355190 | CGGCTTTCCGCTTGGCTGGAGACACGCAAACTGAATCTGCAAGTAATGCA |
| 287 | 1 | 203599089 | CGGCGGGTTAGGAACTCGCTGGCGTCAGGCTGATTCCAAGGCCCCCAAAC |
| 288 | 5 | 176107427 | CGCTCCCCTCGCCCTGCAGCTCGGCTGAGACGCCTCCCTGAGACACCGCC |
| 289 | 4 | 85417416 | CGCTTCCCTAACCTGTGTCCAGGCAACAAAGGGGTCCTTGTTTTTGCACA |
| 290 | 8 | 17271322 | TAGACCCAGGACGGTCTTGTAGGCTCAATGTGTGTGTTTCATCCCAGGCG |
| 291 | 11 | 86383696 | CGCACTCTCCCAACATTCGGGGAAGGGCGTGCCTTCAGGATTTGGAGTAG |
| 292 | 8 | 70945457 | ATGAATGGATGCTCCTCCCAGGTGGCACCCAGAGCCTGGCCCCATCCACG |
| 293 | 10 | 129534613 | CGGACGCACTGCAGAGACGTCCCTGTTTTCGGCGCGTCCTCCGACACCCA |
| 294 | 17 | 33787739 | GCGCGGGGCAGTCAGAGTCCCCCTACTTGAGCGGGTTGTGGCGGAGAGCG |
| 295 | 6 | 154363173 | CGGTGCATCTTACTGAGAACATGTTATTATGCTTTATTCTCGGTACTGAA |
| 296 | 8 | 55382901 | TTAGCTGCTGGTGCGGGGCAGGCCTGGGGCAGCGCGGGGCGAGGAAGGCG |
| 297 | 12 | 48396670 | AGCGTGGAAGGCGGCGGGCTCAGGCGGGCTTTCCGGTCATTAGCGCAGCG |
| 298 | 5 | 140220810 | TGTTCAAAGTCTTTTCTCTGACAGCATCTGTCTCTAAAGGCCGAACAACG |
| 299 | 3 | 125899754 | CGGCTTGGCGAGGGTCTCCTCTGACCCACTTAGCCTTGCCAGCGCTCAGA |
| 300 | 6 | 26250744 | AGTACCAGGCCTGTAGCGGTGGGGCTTCTTCACGCCACCGGTGGCTGGCG |
| 301 | 1 | 53685388 | GGTCAAAAATCCCAGCATCCTTCTGTACCTCTCAAAAGCCCTGTTTTTCG |
| 302 | 12 | 11653278 | CCCCGCGGTGGGTGTGTGAAAGTCAGGGTGGCAGTGACGCGGCGCCTGCG |
| 303 | 3 | 183382726 | GCAACTCTGATCCAAATATTTAACTGTGAGTTATAAATCCAGACATCACG |
| 304 | 3 | 147098585 | GGCGCGCCTGGGCCAGGCCGAGAACAATGCACGTATTAGGATATCAGCCG |
| 305 | 8 | 38965252 | GGAACATGGTGCGGCTTCGGGGCTGCCAGCGGGAAGCCGTTGGGAAGTCG |
| 306 | 7 | 121946608 | CGCGAAAATCAGTTCCGCCTCTGCCCCAAGAGCCAAGAGGGGTGGAAAGG |
| 307 | 4 | 154681197 | CGAGGCCGCTGCTGGGGAGGACGCTGGCCAGCGTGCAGGGGGCGGAGGCG |
| 308 | 1 | 34642519 | CTCGCCGTCGTCGAGCGGGGGAATTCTTTCTGTGGGTTTCAAACGCAACG |
| 309 | 19 | 17600122 | CGCCCTCCTGTCCACCTGCTGGCGCAACCTGCTCCCCCGTCTCTAGGGGT |
| 310 | 2 | 3502790 | CGGGCCGGTTGTACGCTGTCCACACCGGTTCTCCCACAAACAGCCGCATG |

| | | | |
|---|---|---|---|
| 311 | 5 | 170736335 | GGCGGGCGCCTCCATCCTGGGCGGGCGGAAGGGCTGGGCTGGGCTGGGCG |
| 312 | 10 | 134921759 | GGTTGTAGGTTGTGTGTGAATCTGGTCCCTGGCCCCAGAGCTCTGCCTCG |
| 313 | 6 | 100904316 | CTCGCCGAATTGCAAGAGAATCTTTAGATCTTGTTCCCACTGTAATCCCG |
| 314 | 6 | 28956577 | CGCCACACACCCCGAACCCCCAACACGTTGCTTTCGCACTGATGGGAGCA |
| 315 | 17 | 80545125 | CGGTCCACGGCCAGGTGAACAATGGTAAGACATGCTGGTCGGTGGCTCCC |
| 316 | 16 | 49312033 | CAGAGGCGGTGTATAAAACATACGCTGCTTCGGGAACTGCGAGGGGGACG |
| 317 | X | 150867810 | CGGGGCACAGAATCTCCCTACAATACCGTCTTTGGATGGTGGGCATATCT |
| 318 | 12 | 75728458 | CGGCTGGGGCAACCCGATTTCCCCACGCCCCCGCACCACGCTCTCCCTTG |
| 319 | X | 119133091 | CGGTTGGATCTTGCCTTTATTGAACATTTTAACAATTTGGTTACTGTGGA |
| 320 | 16 | 27523174 | GGGACCGACGTCATGGTTCGGTGCCTCAAGCTGCTGAAGGAATTTAAACG |
| 321 | 19 | 39694831 | AAGTGCGCGTCCCGTCCGAGTGCTTGAGGTCGGCGGAGGCGGGGCAGGCG |
| 322 | 14 | 101193397 | AGGAGCAAGAGGACGCGCAGGAGGGCTTCGGTCGCGGTCATCTCTGGGCG |
| 323 | 19 | 46800467 | CGCCCCGGGGCGCGCAGTTGGAGGCACATCCCCACCGCACTCTCCACCCT |
| 324 | 2 | 4931115 | CGCCTCTAGCTGGGCTGTCATTTGTTCTCAGGAGAACACCCCGTCCTTCT |
| 325 | 14 | 88791298 | CTGAAGAGTCAGAGTAGCACCATCAGATTGGTACCAGTTTTGCCTGAACG |
| 326 | 15 | 58357987 | AGGCAGGTGTCAAGCTGGGGCTCTTCTTATTGGACGTGAGGGCCGAGGCG |
| 327 | 7 | 15726821 | CGCGCCTAGGTTTTCGCAGCTGGTTTCTGTCGGTGGGTTGTTAATAGGCT |
| 328 | 6 | 26271587 | AGTTTTTCTTTCAGGTAGTCTGAGATGGCCCGCACCAAGCAGACTGCACG |
| 329 | 7 | 32110145 | CGCCCCCAGACTCCGACTTAGAGCGGACTCCGATCGCGGCAAACCATGGC |
| 330 | 17 | 32596886 | ATGTAAGGGATATTCCCCTTTTCCCTTTGGAGTGGTATCCCTAGGACACG |
| 331 | 22 | 19743759 | GTAGTTGTGGGTGAAGCAAACCCTAATTCCAGAAGTTTTTTTCCCTAGCG |
| 332 | 7 | 150896413 | GAGCCAATGAGGCGGGAAAGTGGCTGCGAGAGCGGCTGCCAACTCGGCCG |
| 333 | 4 | 1342161 | CGCCACACGCCCCGACCCCAGAACATGACCACCCGTCAGAGCCGGGTACT |
| 334 | 19 | 3507867 | TGTGTCAGGGGCCCGTGTGTTTTGGCGACACCCGATTGTCATTTCCTTCG |
| 335 | 7 | 113722758 | TTCGTTTGAAATGGCCTCTGTCAGCGCATCTTGCGCGAAATAAAACACCG |
| 336 | 6 | 101841158 | CGGAGAGGCATAAAAGCCATCACCCAGAAGCAAATGTGTTTGGGCTCCTT |
| 337 | 14 | 101317622 | CGCGGGAGTGGCCAAGCTACCCAACAGGCTTCACACCACTCGTTCATGCA |
| 338 | 4 | 9783198 | CGCGCTCGACTCCCGCCCCACCTGTGCCTTGGCGCCCTCGCTCACCGCCC |
| 339 | 14 | 51290751 | CTAAGATCTGCTGAGAATCCCCAAGGTGCTCCAGGATTTTCAGTGTTGCG |
| 340 | 12 | 115131789 | CGTTAGGAAGATCTAAACTGAAGCACTAGAAAGGTCTGTGACTCTGTTCT |
| 341 | 12 | 85671811 | CTTGCGTGAGCAGTAGCCTCAGCTCTGCGTTCTTTGGACACTTTCCAGCG |
| 342 | 7 | 91687463 | CGGCAGCTGTATCTTCTGGGATGGATTCTTATAGCACCTATTTGGCAACT |
| 343 | 13 | 45008447 | GAAATGGTTTAATTCATGCTCAGGAGCTGTGTGCCTTTCCATCCCTTCCG |
| 344 | 16 | 3163755 | CGCTGCTCAGAAAGAAAACCTCACTGATAGGACCTCCACCCAGACCTCTT |
| 345 | 12 | 123446272 | TGGTAGTAAAGACAGCGGCTGTTTATTAAACTCTGCTTATCTCAGCTTCG |
| 346 | 3 | 75958299 | CGGGCTGACACAAATAGGCTTCTTCAAGAGTTGAGGAAGAAAACAAAATC |
| 347 | 21 | 42688886 | TCCCGCTTTTGCTAAGCGTGGCGGGTCCCAGGCAGGGTTGGGGAGCGACG |
| 348 | 6 | 36628713 | CGGCGCTGGACTCCCTCCAGGTCACACCGCCTGAGGTCACACCGTCTGGG |
| 349 | 1 | 203596940 | CACAGAGAAACTGCTGAGGTTTCTCTGCAAAGCTAAAGAAATTTAATTCG |
| 350 | 2 | 242811830 | AATCTGGTTGCCATGGAGACCATGCTGTTGTCAAGGTGGCTGATTGGCCG |
| 351 | 10 | 43393728 | CGCCCTCCGTCCCACGGTTCTGCTCCGCAGGGAGGCTCGGCCCCATCTCT |
| 352 | 2 | 20548412 | TCCGGAAGGCAAATTATCTTTCCACACCAGAAAGGTTTATAAACAAGCCG |
| 353 | 16 | 30661534 | CGGAGCCACAGGCCGCACCAGCCCGTCTTTGATCCCGGGTGTTCGGACTC |
| 354 | 6 | 166422671 | CCGCGCGCTTCATTTTTATCAGGCTCCTGGGTGTGTGCAGGGAGGGAACG |
| 355 | 3 | 164911744 | CGCTTTCAGAGGAATTTTTTTAAGGCTCAGCTGACAGCACTACAGTCCTG |
| 356 | 20 | 36040903 | CGCCGGAATCCCACCAGCCTGCAGCCACGGTAGGCAAGAGCCTCGGGGAA |
| 357 | 3 | 128998642 | TGTCACTTACCTAGAGATGTTCTCACGAGAGCTTGCGCCTACCAGGATCG |
| 358 | 10 | 25241260 | TACCGGCCGGAGGACACGGGGGGACGCTGGGAACTCGGGGTGAAGGGACG |
| 359 | 6 | 26273611 | CAAGTACACCAGCTCCAAGTAAACTAGTTACCTGGGTAAAAGCGCTAACG |
| 360 | 1 | 158027914 | GTAATGTCCTCATAGATTGCTTGTATCTTCAGCTGCGTCTGTGGCCTGCG |
| 361 | 16 | 23847556 | CGCCCGCAAAGGCGCCCTCCGGCAGAAGAACGTGCATGAGGTCAAGAACC |
| 362 | 17 | 75315081 | GAAAAGTGTTTTGTCTGAAGAGCAGAACTGTCTTATACCAGCCCTGTGCG |
| 363 | 13 | 112711380 | CGGGCGGGCTGAAGCCCCCGCGCAAGTTCGAGACACACTTACAGTTAGTT |
| 364 | 5 | 140219613 | CTTTCTATTGACTAATTGTCAAGACATTAGACTTCCATCTGGGAGATACG |
| 365 | 12 | 122017052 | TCCCTTCAGTGCTTCCGAAGACACCGGAGGGGAGTACCCCGCGTAGAGCG |
| 366 | 10 | 102997382 | TGGCCTTAGCCCGGACTGTGAGTCTCGAGCGGCTTCCGAGAGGCAGCTCG |
| 367 | 1 | 200010283 | CGGCCTCACCCAGCGACCCCAAGGGTAGGCCCTCTCCGGAGCTGCCGGCT |
| 368 | 7 | 154087975 | CCCTTCTGACGACTGTACGATCTGATGATAAATCATGTGTTTGAGACTCG |
| 369 | 18 | 5197327 | CGGCGACGTACACTGCTGCTTAGGGAGCAAAGCACTTCCACCGCTTTCAC |
| 370 | 5 | 141071438 | TAATATCTATTAGGAGCTTCTCAGCAGGACCAGGACTTTAAGGACTTTCG |
| 371 | 6 | 100903909 | ACAGGTTAATAAACAACCTGTCCGGCACAGGTTAACAGGAGCCTCTCTCG |
| 372 | 16 | 3211656 | CAGAGGTGGCCACCCCTGTTCCCCTCCTGGCACCATGATGGTCAGGACCG |
| 373 | 19 | 51568260 | CTAGTGATCGCCTCCCTGACCTTGGCCTTGTCAGGAGGTAAGAATGCGCG |
| 374 | 15 | 42387193 | GCTGCTCCCTGGAGCTTGCTCAGGTCAGGTGAGGAGGATACACTAGCCCG |
| 375 | 1 | 11958326 | CGCCTAAGCCTGGCAGAGGCAAAGCGCTAGACGGAGCACCGCAAAAGCCC |
| 376 | 10 | 94452236 | CGCAGAAATATCTCTCTCCGCCCGAGAGGAAGCGTCTGGCCAAGATGCTG |
| 377 | 17 | 75316233 | CGGATGCATTCGTGATTGCAACAGATTGAAGAAATTAGACCAGACAAAGA |
| 378 | 7 | 133662267 | ATATTATTTATATAGATTGTCCTCCCACTCGTCCTCAGCCTATAGGAACG |
| 379 | 8 | 96148290 | CCCTCTCCAGAAATCAATTAATTTCTCCCATGGTATTACTGTAAATATCG |
| 380 | 8 | 72756882 | AGAGCCGAGATTTGGGCTCTGAAGCCCGACGGCCCTTCAACATGTCTGCG |
| 381 | 12 | 21926476 | CGCGCATCCGAGACCGCCTCCCCAAAGCCCGCTTCATCGCCAAGAGCGGG |
| 382 | 6 | 100915805 | GACCCCTCCCCGCAAAAGGCAAAATCAAGACTACAGAAGGTGAACTTTCG |
| 383 | 6 | 78176806 | CGGTGAGCTGGCTGCGGAGGAACCAAAGAAAAATGAATCCTCGAGGAACC |
| 384 | 10 | 13392131 | CCTCTGCGGCAGAGAACTGCTTCCCGGTGAGCTCGTGGTGGGTGGAGGCG |
| 385 | 10 | 100996070 | ACCATGCCGTTGTACCGTCTGGGTACAGAGCAAAGGGTCTCGTTTTTACG |
| 386 | 12 | 3862423 | GGCCTGCAGGCGCGACCTGAGGCCGCCAGGGAGGCTGGGTGGCGAGGGCG |
| 387 | 1 | 46951477 | CTGGGTGGTGGGCGGCTTAGACCTAATTCCGGAGTTAATGGCGGCGGCCG |
| 388 | 16 | 49893122 | CACGGGGCTGCCCTTTTCCACCCCTAGGGGCCTGCTGAAGACTGAGGACG |
| 389 | X | 134125181 | CCTTGGGCTCTGGGCACTATGTGGGAGGCAGCATCAGGTCCATGGCGGCG |
| 390 | X | 51139066 | CGCGAGTTCGGAGCAGCGCGTGTATCTGTGGGTCTTTGGGGCTGGCCTCG |
| 391 | 16 | 73578307 | CGGCAGGCCTGATTGGTGGGCTTTTAAACGTGACTAATGAAGAAAATTTA |
| 392 | 11 | 95437294 | AATTAAACCCATAACCAAGGAATATACTAGGTTCAACTATGTACTTAACG |
| 393 | 2 | 106227786 | TCAGCAAAATAAAGGATTCACTGCAATTCATCCTTCTGTGGTCACTGCCG |
| 394 | 7 | 137028442 | TTTTTGGACTGGAAGGCGGGGAACCTGATCCTGCCTTTGACTCAATTACG |
| 395 | 11 | 68781279 | CGCCATCTGATTGCTATTAGGGCTGGGGAAAAGGGAATTCCTGGTTCCAT |
| 396 | 13 | 28550046 | CGGCAAATAGCGACATGCTCTCGGTTTCTGTCGGCCTTGGCCTGGCAGAG |
| 397 | 11 | 68924853 | CGGCCACCTCCTCCCGACGCTTTCAGCAGCCCACGCGGCTCTTCCGGCCC |
| 398 | 18 | 31021003 | ACAGCAGTGAGGTTAGAACGTGAGGGGATCACAGACTTCGTCCACCTTCG |
| 399 | 14 | 62584513 | AAATGTAAACCTTAGGATTGGTTCTTGGGTGAAGAGTCCGCTGCTCTTCG |
| 400 | 7 | 158114659 | CGCGGAAGCCTAACCCCCAGGTGCTCCTCGATGACGGCTTCGTGCCTTCT |
| 401 | 14 | 85997753 | CGGGGCTCGCTCCCGCACTTTGAAGGCTGCCGCGGTCTTTCGTCATTTAT |
| 402 | 20 | 19915874 | CGCACCCATCACCTAACCGTGTCTCTGGTCACCTGTGGGTTTCAGCGGGT |
| 403 | 1 | 103574523 | CGGCCACACCGCTGGAGTCCTGGGCTGGCAGCGGTAACCTTATCCTTGTG |
| 404 | 8 | 21961713 | GAGGACCTCGCCGCTCTGAAGAGCACCGTGCACATGTGGGTGCACAAACG |
| 405 | 11 | 46969673 | CGGCTCCATGGTTCTCATGCCCCAAAGTCCTGCAAATTGACACGTAAGTA |
| 406 | 1 | 2324396 | TCAGAGGACTGAACAACACCAGAGGCGTGAAGCTTGGTGCACGGCAGCCG |
| 407 | 1 | 149192846 | CGGCACTCATAGATCCGCACGTCCCGAGAAGCCTCTGCCATCCCAACCCG |
| 408 | 4 | 85405132 | ATGGCTGGACTTGCTGCGTGGGTGATCCCAGGACTAAGGCAATTAGAACG |
| 409 | X | 47483257 | GGCTCCTCCAGGGTTCCGGCTGGTGGGAATCTAGGGAGGTCCAGGAGACG |
| 410 | 17 | 75315503 | CGGCTAGAGACTCACTGACTCATGCGTGTGTGGTAGGAATCTTCCAGGAA |
| 411 | 5 | 140624869 | CGTTTCGAAGCAAAGACAACGCATGGTGGCGCTGCAGGCTAAGGCTTCAA |
| 412 | 2 | 220283442 | TCAGAAACTCCTGGTTCACCGCGTCGGCCAGTGAGAAGTCCAGCAGCTCG |
| 413 | 2 | 119067552 | TAATACAATAACAGAGGCCTCCGCTCCATTTGCATTAGGTGAGGAGCGCG |
| 414 | 8 | 130996123 | CGCCGGGGCCCCGCCCAGGCCCTAACGAATACATCCCATTGGCAGGAACC |
| 415 | 19 | 55047721 | CGCCCAGCCTCGCTGTTCTTATCTTGGCAGCAGATTCCGAATGTCGGCTG |
| 416 | 14 | 60386272 | CGCCCAGTGAGGTCCAGAGAAGAGAGGGACCTTCCGAGGGTCTTCTAGTC |
| 417 | 1 | 197887649 | TCTCCTTAGAGGGACTGAGTCGGGATGCCAGAAACCCTGAGATTCTCTCG |
| 418 | 17 | 33701350 | CTGCGGCTGAGGCAGGCTCCGCAGACCCCGAGCCAGAGTGGGATTTAACG |
| 419 | 18 | 56941473 | GATGTGCTGCCGGGCTCAGGTCCCGCAGGAGACCCACCTGGAGTTCCTCG |
| 420 | 10 | 101875035 | CGGCCCCTGAACTTTTGCCTGCACCGGGGCCGCCCCGCACCCCCCGCCAA |
| 421 | X | 50557066 | CGTAGAGCTGGAGGGCGATGGTGGCGGAGCTGCTGGGGGCGGAGGCAACG |
| 422 | X | 151620975 | CGCATTTGGGGACTCACCTTTGGAGAACTTTTGAAATGTGGCAGTTTAGT |
| 423 | 17 | 4692287 | CGCCCCCTCACCAGCGCCTAGCCAGTCCGTGTCCCTCCGCCTCCAGGAGG |
| 424 | 6 | 6002766 | CGACCCCGCCCTGCCGCCCACTGCCCTTTCCTGCGAGACCCTGGCTCCGC |
| 425 | 1 | 62660861 | CGCGCCCTTCGCGCCTGGACCAGCCTTATCCGGGGCACCCTGGACCTCAC |
| 426 | 16 | 84628969 | ACGTTATGCTTACAAATGTTTGCTGCTAGAGATCTGGCACGACAGTGACG |
| 427 | 1 | 47696788 | CGGCGAGCGCTGCTCAGCTTCAAGCCTGGAGGAGCCTCCCACTCACCAAC |
| 428 | 6 | 26273193 | CGTTTGGGCTCACCAGCATTTTCCTGTGGTCATTTGACGGTATCACTTCG |
| 429 | 11 | 6817280 | CGGCTGCCCTAAAATGAGAGTTTGTCTTTGAGTTGGATCATGACTCTGTT |
| 430 | 11 | 132513895 | CTTGGAAATGACAAACACTCCTCGTGGTCGTCTGATCCCAGATCATTGCG |
| 431 | 16 | 88717134 | GAGGAGTTCTGAGACCCCCAGTAATTCCCTTGCAGACCCTGCGGAGCGCG |
| 432 | 18 | 67069959 | AGTAGGCTGGGAGGCTGCAAGAGCGAGTGCGGAGCTGGGTGACAGCAACG |
| 433 | 9 | 140081972 | AGCCCGCTGTGTGCAGGGTGCAGCAGTGACAAGCAACAGTGCTGGTGTCG |
| 434 | 4 | 44654206 | AATGGGATCTAGTGCTTCTTACCTGATGGACAGGTTCTATACCATTATCG |
| 435 | 6 | 100897081 | CGGAACTTTGTGAACAGCTGGGGACTATGCTTAAAGACAAAGTCTGACTG |
| 436 | 22 | 29976613 | CGGAGGAGCTGACTACCAGAAGCCCCCAGTGGGTGTTAAATCACAAGCTA |
| 437 | 4 | 183062301 | CGGTCCCATTATCCCGAGCACGACCGGCTGCGTTCACACCGCAAAGTCAC |
| 438 | 3 | 181413810 | CGGGTGCTTTTAAATACACCTGGGTATTATGAGCATGTAATCTGGCCTAA |
| 439 | 1 | 165324100 | CAGAATCTCTGGCTTCAGCTCTGCAAGACCCGTGTGTGGACACTCTTCCG |
| 440 | 10 | 134143489 | CGCGTTCCTTGTTGAGTCATTTCCTGTCTTGATTTTAGGACCTTCCTCCG |
| 441 | 5 | 115297989 | CTTTGGGTTCGAAGAGACAGACCCCACGGAAGATGCCTCCAGGGTTGGCG |
| 442 | 2 | 238599734 | CGCGCCTGTGAGCACTCACCTTCCCGCCGTGACCCGGGCCCAGGGCTTCA |
| 443 | 11 | 5841482 | GTACATGGCACTTGAGGTTCACTTTGTCCAAACCAGTACCTCTTATCTCG |
| 444 | 3 | 147111660 | CGCTGTGACCGCCACAAAGTGAATCCTTTCGGTGCGGACAGTCGCCTTCA |
| 445 | 20 | 21490238 | CGCAGCAGCCCAGGGACGCGATGACACTTTATTCCCGCGTTCCACACCGC |
| 446 | X | 136630705 | ACCTGGCAGGGCTGGGGTGGGGATTGCAGCTAGTCCATGACCCAACTACG |
| 447 | 15 | 37390176 | CACTCACACACAGTAAGCTGGGTCCGGGGGGTCAGTTTACCCATAGATCG |
| 448 | 2 | 45179787 | CGGCGCCAGTGTGTCCCGGGCGAGGTAGGGGCAGGCAAAAGGCGGCTGCG |
| 449 | 7 | 154796431 | TGTAGTCACCTGATTAACAAGAGCCAGAGTTCATTGCATGCTGATCAACG |
| 450 | 13 | 36919738 | CGCAATAATGCTGCGTCACGAAACTGCGCATTCCAAGAGAAACTTTTCTC |
| 451 | 7 | 121942010 | CGCTGAGGCAGCGGCAGACTAGAGCTCCTGGTCCGAGTTCGTCTGCAGAC |
| 452 | 7 | 15727525 | GCCCCGGGCAGAATAGGTGACTACAGATATTCTCCTGATGCGACAGGTCG |
| 453 | 22 | 38214979 | TCAGAGCCTCTGATTCTGCAGAACGCAGACTTGCAGCTGGTCCACGGCCG |
| 454 | 2 | 175191374 | GGACTGGGGACTGCCCCCAACTTCCGAGCGGCAGAATCTGGCGCTTGGCG |
| 455 | 2 | 239007828 | CAACCTCGCTGAGTTGGTGCAGCTCCAGGTCATTCCTGGGGTGGGAATCG |
| 456 | 3 | 192126165 | TTTCACACATTTTGTCGCCACTGCCCAGACTTTGACTAACCTTGTGAGCG |
| 457 | 10 | 80981129 | CGCCCAGCCCCCAGCTCTTTGTTCTGGCGCAGCCTTTAGCCTCAGAGCGC |
| 458 | 12 | 25102274 | CGGATACGCACCCACAGTGGCTGATTCGGGGGTAACCGTGTCATTTGCTT |
| 459 | 1 | 200010788 | GCAGCCGAAGTAGGCGTTTGGGGAGGCGAACTCATCGGGGCAAAGAAGCG |
| 460 | 8 | 67085823 | CGGAACATGTTTGCTTATTTATCCATAGTTCTCCTAGGAAGGACGGTGTT |
| 461 | 19 | 2251061 | CGTGGCCGCTCTCAACTCCTCCAATTGCGGGTTCCAGGCCATCCGCGGAA |
| 462 | 15 | 60289532 | CGCCCCCTAGCCATTCTTAGCTGGGGGAGGCCAGCACGGATCAGCCGCTT |
| 463 | 3 | 16554466 | CGCCTCCACCCTGCCCCACCCAGGCCGCGCGCACCCCGGAAACCTGGACT |
| 464 | 1 | 166917158 | CGCCTGCAAGACCGCATTTGTTCTCTTAACACAGGGCAGTGAACGGCCGC |
| 465 | 16 | 51190201 | CGGAAAGTCAAAATCGCAAGCACTTGCAGTCGCTAGCGAAGTCGGTTTCT |
| 466 | 2 | 100722584 | ACAGGTAGAAGACCAAAAGGCCAATGAGACTTCTTTCCTATTTGAGATCG |
| 467 | 1 | 45250181 | TCTGCTCAGGGTCGTCGCTCATGCTGCGGGCGGGAGGGCGGGCTGAGCCG |
| 468 | 7 | 19813299 | TACGTCAACGCGGGCTCATTCTGAGAAAAACTTTCTTTAGCTTGTACGCG |
| 469 | 17 | 37123843 | ATGAGACGGAGGGCACCATGGGAGCTGGCTCTCTTGCTATTCATAAAGCG |
| 470 | 1 | 18963958 | CTTTCAGGTTCCAGGGCACTCGCCACTTTTTCCAAAGCCATCAGTCTGCG |
| 471 | 11 | 32459428 | CTCTTTAGTCGGAGAAGTCCAAGTCGGGCGAGGGGGCACCCCGGGGTTCG |
| 472 | 20 | 26190328 | TGCGTTGGGAAGGCATGGGGTGCGGAACCCGGCCTGACCTCGCTGTCCCG |
| 473 | 2 | 27806529 | TTCAGAGGTCCCTAGCACTCACTCGTGTGCTTGTGATTTGCTAGACTTCG |
| 474 | 1 | 116710943 | CCCTCGCTGGGGCACCTGGGGAGGGGGCGCTGGGGCACCTGGAGAGGGCG |
| 475 | 16 | 54971018 | CGCAGTGATCCCGTGACCCCTGTTGCTAGACATCCAGAGGCTTCTGGTCC |
| 476 | 7 | 100488942 | TGCTGAGCCCCCGCCGTGTACGGGGGCCATTGTGGGGCCTTGGGGTCTCG |
| 477 | 15 | 89949694 | CGGAGAAAACACTTCACCCTGGAACCCTAACTCAGGTCCTGGCAAAAGAT |
| 478 | 11 | 122935222 | TATTTAAAGCTCAGAATATGATAAAGGTCCTGTCTCCACCACTTCAAACG |
| 479 | X | 150863969 | GTGGGCGGGGACAGGGCCCCTGCTGGAAGAGGCGCAGCAGCGAGGTGGCG |
| 480 | 4 | 85424288 | CGCCGTAGGATCTTGCGTTCGGCGCTCTCCCAGTGGAGGCCAACACCAAA |
| 481 | 10 | 45914450 | AGGGGTCGGGGGCTCACACGCCCTGCGATTTCCTCCCATGAGAATGGGCG |
| 482 | 5 | 118677172 | CGATCCTTGGGCTATAAGCCATCTGACTTCAACTATCACTGCTGTGTTTG |
| 483 | 7 | 1121657 | CGCCGCCCCGCAGGAGCACGCACTCCCACACAAAGCGCCTGCAGCTGACA |
| 484 | 14 | 38068340 | ACAAGGGGTCACACGTGCGTGTGCCTGGCCTTCGCAGGTGGATGTGTGCG |
| 485 | 1 | 152681887 | CGCCCACTAATTCCTTTGGTGCTGCTCCTGGTCCAGGTCAGCAACAGCCC |
| 486 | X | 151562205 | CGTGGTTTAACAGTCAAATATTAGTGTTAGTTGCTCATGCACCATGATTG |
| 487 | 8 | 25908279 | CGGATTTCAAGAAGTCTTTGACGGTGCAAACTGGACTTCTAATCATGGGA |
| 488 | 5 | 158524404 | ACGCAGTGCGTGTCAGGTAGGAGTAAATGCTTATAGAGTTTCATCTCGCG |
| 489 | 14 | 70014873 | GCAGCGTCAGTCGTCTGCAGGAATTTGTGCTGGGGTAAATGCGCGCTTCG |
| 490 | 7 | 44163975 | ACTTAGGAAACAGGCCCAGGGAAGTAGCAGAACAGTAGCGCTAGAGGACG |
| 491 | 21 | 46143498 | CGGCCCCGCCGGCCTCGCACCCGGTAATTGCAGCTTCCCCCCGGGGCCCT |
| 492 | 22 | 28839249 | CGCGGGCTGCTACAGAATCCCAGCCGGGCTTCTACCATCCAAACCCCTTT |
| 493 | 2 | 182547599 | TCAAGAACATCTGACTCCACCATAATTCTCCTGTATTGCACAGGATGTCG |
| 494 | X | 107179321 | CGCGCCCCCAGCTTCGCGGCAGCTAGGTCCCTCCCCAGCGCGCTGAGCTC |
| 495 | 14 | 96180842 | ATCCTGGTGTCGACTGTGAGTTCCCAGCAGAGGCCCAGAGTCCCGGTCCG |
| 496 | 19 | 42445244 | CGCCTGGCCCCGATCAGTCCCCACCAGGACCACCTATTTCCCCAAGTCGG |
| 497 | 14 | 36983694 | CTCCTCGCCGTCTACAGTCGGCATAGTTTGAAGTCTGTTTACTTGTCCCG |
| 498 | 3 | 158361858 | CGATGTCAGACATTTCATGTCCTGTCCATGTGGAAGACTGCATCCTTGAC |
| 499 | 4 | 81106663 | TGAAGTGGAGCAGACTCACCGGCAAGGAGCCCGGAAAATCGTCAGGGGCG |
| 500 | 10 | 91295159 | GGTGGCTCAGGTGACACGGGCATCCTTCAAGGAAGGGCGGCGGGACGGCG |
| 501 | 1 | 197889248 | AATCCTCCTTGCAGTAAATGCTACCGTCCTTGGCAAAGCAGGTGAGCTCG |
| 502 | 2 | 201450506 | ATAAGACCATCAAAAGCAATGTCCCAGGACTTACAATGTTTGCTAAGACG |
| 503 | 3 | 48056973 | CGTCGCCAGTGTGGGAGATCATTTATAATCTTGATGGATACAGGTATAGC |
| 504 | 12 | 132969187 | TGTGGGCTGTGCCAGAACCCTGGGAAGAGCAACGTGGTCGGAGGGTCCCG |
| 505 | X | 110339005 | CGGTTTCGATTTGGGACACCACCTGTCAGCCAGGCAGGTTAAACTGACGG |
| 506 | 4 | 81189927 | CGCACCAGCGAGTCCCAGGGCCTCCCTCACACTCCCGAGAGGAATCTATT |
| 507 | 1 | 38942313 | CGGCGCCCCCTCATTAGAGGGCGTTTGTTAGGGAATGTTTTCCCAGCGAT |
| 508 | 6 | 108489977 | CGACCCAGTCCTGACCCGGCCCGCTGCGCGCCGACCTCCTAGCTGCCCCG |
| 509 | 3 | 38036060 | GGGGCAGTCTTGGGATGGCCATCGTCCACATCCCTGAATGGGGCAAGCCG |
| 510 | 17 | 6922320 | CGGGGGCCTAAAAAGACTGCTTACATACACCACCTTGGATCTTTCAGATT |
| 511 | 17 | 75315868 | CGTCAGGAATTTGACTCTGTGAGTTGGTTTCCAAGAGTCTAAGTTAAGCA |
| 512 | 15 | 78933630 | CAGGGGGCGTCGGGGCGGAGCAGCGGGCCCAGTACATCCCTGAGTGGGCG |
| 513 | 3 | 172635656 | CGAGTGTGGCCTTTCAAAAAGAAGTGCATTCTAATTTTAAGTTGAAGTTC |
| 514 | 3 | 128764824 | TGTATTCTTGGGGTTCGGGCTGTATTCCTGGACCCCGGCTCCCGGGTCCG |
| 515 | 6 | 134210946 | CGACAAATACGAGAACGGGTACATTCACCCGGTCAACCTGGTGAGTGCTC |
| 516 | 13 | 88326918 | CGCTGATGTGCTTTAATAGCGTGGTAGTGAAGTCCACCATCAAAGCAAAT |
| 517 | 6 | 101850540 | GCCTTTGATCCTGTGTTGGGACAACTTGGGTTTTCCTGGGTGCCCACCCG |
| 518 | X | 36982194 | CCCTGAATTACGTACACAGCTAGCAATTAAAAATTAACCTGTGGTCCCCG |
| 519 | 11 | 44325823 | CGTCCGGGTGCAGGACGGCGCTCTTACCGCCCCACCCCAAACCGTTGCCT |
| 520 | 16 | 51184355 | TGTTCCGGAGACGGTGCAAACGGACGGGGAAAGTGTCGGGGTCTGGCTCG |
| 521 | 16 | 1013990 | CGGGAGGCAGCAGAGCCATGGCGGCCACCCGCCTCTCACAGGGACCCAGA |
| 522 | 6 | 105405068 | CGGAGTGATCTTCTGCATCAATCTAACATCTTGATTTTGTGCGATCACAA |
| 523 | 1 | 150267044 | CGCCCGGCCCAGAACTTCATTTTTAAGATAATAAACTGAGGGGCTGAGAC |
| 524 | 3 | 79815718 | TCCGAGACCTCAGGGCGCGTGGCAACTGGGCAGAAGCCGTCTCTGCAGCG |
| 525 | 1 | 91171013 | CGCTTTAAAGTGGCTACAGCTCTGATGCACCCTACTTCCAGTGGGAAACT |
| 526 | 1 | 158301038 | CTTTCCTAGGTGCAATTGGACACTAAGGGTCTGGGTCTTTCTGTCTCCCG |
| 527 | 11 | 44332664 | CGCCCCACGTCTGCGCCCAGGCGAGGGGATTTCCTGCCAGGCGAAGTACA |
| 528 | 8 | 57358713 | ACAGCTTTTAGGTAGACGTGGAGGCGACTCAGATCGCCTCGCGGTTCCCG |
| 529 | 11 | 27015872 | GAGGAGTTCCAGTCACCGAGCGAGGGGCGCAAGGGTGGGTGCATCCTGCG |
| 530 | 19 | 36604588 | GTAATAAACACCAGATTCCATGCGTGAGTTTTCTGTGTTTATTTGCCTCG |
| 531 | 5 | 140220803 | CGGCCTTTAGAGACAGATGCTGTCAGAGAAAAGACTTTGAACATTGACCA |
| 532 | 4 | 122686453 | CGGCGCAGACTCTCCCAGCTGGCACCAAAGCCTTCCGCTTCGCCGAGATC |
| 533 | 16 | 30572739 | CGGCCACCTAGACGCGCTCAGCGGCGCCCGCCCCACCCGGATCCCCGAAA |
| 534 | 17 | 75315486 | CGCTCCCTCCCAGGGGACGGCTAGAGACTCACTGACTCATGCGTGTGTGG |
| 535 | 7 | 79083445 | CGCGCTGACTCCCTCCGACCTGGGCTCCGATGGAGCAGAAATAGCGGGAC |
| 536 | 11 | 87908631 | CGCTCCCCAAGTCTCTCTCGTGCGCAGAGCCCAGGCTGCGCTTCCCTGGT |
| 537 | 19 | 39755901 | GGAGCCCCTGGCAGCCGCGGGGAGGGACGTGGCGGCCTGCGTGAGTGACG |
| 538 | 3 | 38036034 | GGAGCCGTGGAGGCGGTGCCTTGGCTGGGGCAGTCTTGGGATGGCCATCG |
| 539 | 6 | 100050812 | GAAAATCGAATTGAGTGCGGGCTCATTCGGCAGCGGCCGGCAGAGAAGCG |
| 540 | 18 | 5630428 | TCTCTCTGCCCTTGCGCTACTTTTTTTCTCCAAGGAAGGTTGTGCCTGCG |
| 541 | 2 | 223154140 | CGATCACGTGAATAGGAGTGTGGCCGCTAAATCCGAATGGCTTCAGCATC |
| 542 | 6 | 134210812 | ACCCAGGGCAGGGTGGTCTTGAGTCTGGAGAAGGCCTTGCTCAGCACTCG |
| 543 | 16 | 51184379 | TGGAGAGTGCTGCTGGCCGCCAGTTGTTCCGGAGACGGTGCAAACGGACG |
| 544 | 14 | 52536175 | GTTGCAGGAGATGAGCTCAGCGCAAAGGGAACCCCGCAGCGGCGAGTGCG |
| 545 | 19 | 39737768 | CGCCCCCTCTGGCAGCACGGAAACCTCCACGCCATTGGCTGCCGAAAGCA |
| 546 | 14 | 29229249 | CGCCTAGAATCAAGGCCTCTGGCTCGATTGCGTTCGGCAAGCGTCTGCAA |
| 547 | 5 | 72529902 | CGGGCGCTTTCATGTCACTGCGGGTTTAGAGCTGATTCAAACTCATTGCG |
| 548 | 19 | 6502763 | CGCGGTTACTGCAAGGATTCTGGCGAATGAGGTCTAAATAGAATGAGAAG |
| 549 | 3 | 147127662 | CGCAGGCTCCAGCGCCCGCCAGGCAGATTCTGCCGGCCCGGCTCGCACTT |
| 550 | 16 | 10274315 | CAGGAAGCCAAGTTGGTAATCCCTTTCTTCTGGTCGCCTTGCTCACCGCG |
| 551 | 2 | 86331859 | AACTCAACATGGCCTAAATGCATATCAGAAACTCAACATGGCCGGGCACG |
| 552 | 19 | 51522641 | CGCCCCAGCTACCCTGGCTGCAGCCACGCCGCGCCCGAGGTTTCCCCCTC |
| 553 | 2 | 17699746 | CGGGCCGCTCTGGTCACGCCCCTGACCCGCCCCTCAAGACCTGGCCTTAG |
| 554 | 14 | 38069206 | GTAGAGCGGGAAAGCCCGAGGATCCTTTCAGCAGCACAGAGCAGAGACCG |
| 555 | 7 | 155259845 | CGGCGAATCCCCGCTTTAGGAATTGCACCTTGGCCCTTTGTCGTCAGTCG |
| 556 | 7 | 96632841 | CGCGGGCAGCGAGCTTCAGGTCTTCTGATACATTGAAGGAAAGAAAGAAA |
| 557 | 17 | 47074883 | CGCCGCGGGCACTTCTCCTGGGCTCTCCCCGAACTCTCCCGCGACCTCTG |
| 558 | 12 | 57618943 | AAGGCGGCGCGCGCCAAAAAGATCTTCGGCTGGGGGGACTTCTACTTTCG |
| 559 | 3 | 172167445 | GTGATATCAAGTGTCAGGGACCACCGGAGGCACTCAATAAATAGAAGACG |
| 560 | 6 | 100894513 | GAAAGAATAAGGCTCAAATGCAGGCCTCTGCAGCCTCCCTGTCATTCGCG |
| 561 | 1 | 197898229 | AGTGGTCGCAGTGCCGGGTGGAGTGAGAGCTCCGCTGTCTGCTGAGGGCG |
| 562 | 1 | 181452707 | GAGGAATGGAGCTTCGCAGAGGTTGCATTTAGATTCAACAGTTCACAGCG |
| 563 | 7 | 49813033 | GAATGGCAGCCCTGGCAGGGTCTGCGGCCGGCTGCGGAGGTGGGGACGCG |
| 564 | 8 | 72761320 | CGGAGGGAAGGGTAAAAAATCTTGCTGCAATCCTTAGATAAATAAACCAG |
| 565 | 16 | 677664 | CGAGCCCTCCTGGCATCTGCACAGGCGGCCCCGCCCATCCCCGCTAGGAG |
| 566 | 3 | 62360464 | CGGCCTGTTGAAAGTGCTGGAGCGTCTTTGAGCAGTTTGTTGTGATACGC |
| 567 | 3 | 78084695 | CGCCAAAAAAGAATAAGTGCCTCCCAAACAACTGAATATAGGAGCTGTGT |
| 568 | 14 | 95239381 | GCCTCCGCGCGGCTGCAGATTGAAGGGGCAACCCGATGGGGGAGGTCCCG |
| 569 | 2 | 177024947 | GCGGCGGGGAGGCCGCCTTGCCGGGGTGCATGAGGCTGCGGAGTACTCCG |
| 570 | 14 | 54423845 | CGGCGACTAGTCGGTGCATGCTTTCTAGTACCTCCGCACGTGGTCCCCAG |
| 571 | 3 | 26663593 | ACTTTAACCTGCAGCCGAAATGGAGTCTATAGGCCCCTTAAAAAAGCGCG |
| 572 | 14 | 97685274 | CGGTCCCGATGACCAGGCCTCTTTAGGGTCCTGGCTCAGGCTGCCTTTGT |
| 573 | 14 | 30218600 | GAACCCCAGGATAGAAGTTTTGTAGCCTATTCAGGTTGGAATATATGCCG |
| 574 | 8 | 146278795 | CGGCTTCCATCTGAGCCCTATAGTCTCCAAACAAGGAGTGCATCAGCTGC |
| 575 | 5 | 150326174 | CGCAGTCACTGTGGTACAGTCCACATTCCGGCTCAGCGCCTGGCGAGTCC |
| 576 | 15 | 74891207 | GCCTGACTGGCTTCGGTTACTTCGCACAAAAAGCCCTTGCCGAGCAAACG |
| 577 | 7 | 14942815 | CGCGTCTTGACATAAATCAAGGACTAAGAGAGACTGAAGAAGAGCGTTAG |
| 578 | 4 | 41875470 | CGCCTTTCCGCAACCTACTGGCTCCGTAGATACAGCCATTCAGCCGGTTA |
| 579 | 15 | 89899729 | TCCAAATTCCTAGACTCTATGTTACACTGCCTTTTAATAATAGTTCAACG |
| 580 | 11 | 66103889 | CGCTTCAGGAAGAGAATCCAGTCCCAAGGCAAGGCACGCACATCCCCAGT |
| 581 | 6 | 10887047 | CGTCATTTAAACGTTCCTTATTTGCGAAATTCCAAACAGATAGGCGTTCG |
| 582 | 18 | 21741492 | CGCTGTCTGGGTCAACATTTCAGGGAGGAGTCTGCCACCAGTGTAATGTA |
| 583 | 16 | 48644408 | CGAGACTGAGAGTGTCACAGGGATTGGACTTGACCTCAGACATCTTGACG |
| 584 | 6 | 19692172 | CCGCGAATGGGGCTCGTGGAGCCTGCAAAGGAGTCTGCCTTTGGTATGCG |
| 585 | 2 | 165812146 | CGCTGCCCTGCCCCGATCTCACAGAGTTATGACCGCCTCGTTGCAGTCCG |
| 586 | 11 | 120302922 | ACAAATGGAATATTAACATGCTCTTAAAGATATCAACAGTTGGTCTCTCG |
| 587 | 6 | 100896760 | CGCGCTCCCAGGTGAGTTCAGTGTGGCTGTGCAGTCACTTTCTTAAGACA |
| 588 | 11 | 66327000 | TAGGCTCTCCTGGGTTCTGTAACCCCGCCGTGGTACCCAGGTCCCATTCG |
| 589 | 14 | 29255119 | CGCTATCCATCATCCATGTGACTGGGCGGAAGTGCAGGAGAGCAAGGGGG |
| 590 | 1 | 197882836 | CGCTGGATACTGTTTTAAGAGCCGCGCCTTTCAAACCGAAGGGGCTCCTT |
| 591 | 11 | 116451176 | AATATTGATGCACATCTGTCCCCCGCTCAGGTGTAGAGGGGAGCTTTGCG |
| 592 | X | 82763219 | CGGCCAATCGCAGCGCGTCTCCGCCTGGCCAGGAGTAAGCCCGGCCAATA |
| 593 | 6 | 108489795 | CGGGGCTGGAGGCTTCCAAGGCCTCACTCAGGCTCTTGGCAAGTTAGAAC |
| 594 | 14 | 105102977 | CGACCACCGTTCCTGCTCCTCTGGCGGGTGTCTTAAATGTGCTCTTGTAT |
| 595 | 12 | 81471938 | GCCGGAGGAGATGAAACCGTCTTGGCTGCAGTGTCGTAAAGTCACCAGCG |
| 596 | 21 | 35898975 | ACTAAAGAAAATCACGTTATATTAGAAGCCTTACCCTGGTTTCACTTTCG |
| 597 | 6 | 26240579 | ACTTTTGCAACTTTGCTACACACTGAAGCCCCTTATATATCAATTATGCG |
| 598 | 3 | 45018949 | CGGCAGGTCACGATTTTTAAATGGACATGTCCTTAACTCACGTTGTGGGA |
| 599 | 7 | 6310773 | CGAAGGCGCCCAGTTGCGAACCCTCCTACAGTCAGGCTGTCTGGAGCCAT |
| 600 | 5 | 140098392 | CGCCTCCAGACAATTGGAGTGTACAGCCAGGGACACACAGCAGTGACAAG |
| 601 | 7 | 156933206 | CTCTCCTAACTGTAGAAATCCTTTGATCTCATAAAACATTTTCATTAACG |
| 602 | 6 | 33170101 | TCTGTCTGTGGGACTGTGGGTTCTCAGTGGAATTGTTGCCTTTCTTGTCG |
| 603 | 11 | 131410879 | CGTGCCACTAACGAACTACAGAAGTATGAATTGACTCAAGGGGGCTGGCA |
| 604 | 6 | 70992454 | CCTTCCTAGACCAAGGGGTCATCTACGGCTCTGGACTTCGATAGGACTCG |
| 605 | 17 | 33700801 | CGTGGCAACAGTCAGAAGGGTGGGAGCTTCTAGGCAATTTAACGCGGCAG |
| 606 | X | 63005366 | CGCCCGCCCAGGAGAAGGCAGCTGCTACCTCTGCCTGCCTCCCGCTGCGC |
| 607 | 8 | 145728507 | AGGCCTGCGCACGGGGAGGCTGTTGTGGCAGCGGAACAGAGACGTGCACG |
| 608 | 8 | 72596351 | CGGGACCACTCCAGCTTGCTACTATGGCCCATTCAAGAAATTGAGTAGGG |
| 609 | 19 | 846179 | CGCCACAGTCCAGCTGCCACAGCAGGACCAGCCAGTGCCCCACGGCACCC |
| 610 | 7 | 79083635 | ACTATATAGATGAAGACATGAAACCTCTCTACACAGTGAACTCCCCCACG |
| 611 | 2 | 38892887 | CCAAGATCGGTCTCACTGTAAGTCTGCTATCTGTATTTTCCCATTCTTCG |
| 612 | 11 | 134143149 | CGCTCAAGTGCAGGCCTTTCTAAAGGAGCTGCTCAGCAGGCGGCAGCCCA |
| 613 | 3 | 77122541 | GAAAAGCAATCTTCAATGAATTTTAAACAATTATGGAGACTATTTCCTCG |
| 614 | 11 | 111847475 | CGCCCTGGGGAAGCCCTGCCAGCCTGTTGCTCCGAAAACCGCATACCAGC |
| 615 | 22 | 19749188 | ACTCCAGAACTGAACCAGAGTTTTCTTGAGGTGGGATTTACTAGGAGTCG |
| 616 | 2 | 175191045 | TGTTTCCTAATTTAAGAGGACCTTCGCCGGGCTAGAACGTCAGGGATTCG |
| 617 | 1 | 26797576 | CGTCGTCGTCGCCCAGCAGCAAGGGCTGCAAAATAGTAGAACTCGTGGTT |
| 618 | 16 | 30573013 | GTAGGGTGCAGGAGGTTCCACGGACGCCTCGACGACCGGTAGTAGCTTCG |
| 619 | 13 | 114109729 | CACAATGACATGTACAATGTCACTGAGAGGAGCTGGCGTTGTTGATTCCG |
| 620 | 7 | 136554352 | CGCAACCCTCCAAGCCCCAGGATCCAAAGTTTCCGTTTGAGAATCTGCAC |
| 621 | 14 | 62584600 | TTCCCGTTGACTTCGCTGCCTATGTGCAGCTAGGCTGGAAGCAGCAATCG |
| 622 | 19 | 2250901 | TCGGACCGCGGCAGCAGGAGCAGGGCCGGGGTCATCCGTGTGAAGCAGCG |
| 623 | 7 | 30326592 | AATACGTAGTAATTTATCCGTTCTCATGTTCTGTACTATCAGCACACACG |
| 624 | 14 | 36983542 | CGCTTCAGATTGCGTTCTAGGTCTCTTCCCGCAGCTGCGACAATTCCCCC |
| 625 | 8 | 99439948 | CGCCCACCGAGACCCCTGGGGTGGAGCTGTGCTAATAGAAACATACCCAC |
| 626 | 6 | 74826447 | CGGTAAAGAAGAAGCTAAATCCTTCCTAGACAGAAGGATCCTGAAGCTGA |
| 627 | 6 | 84140928 | AGGGCAGCCTGCGAGAAGCGCTGAGTCATGGCAGGAGATCATCCGAGCCG |
| 628 | 1 | 200011684 | CGCCACTGATTAACTTAAAACAAACTCGGCTGCGTGTTTGTGCGCCGTTG |
| 629 | 12 | 53359155 | AAGCCCCTTTACGTCGGGGTGGGATTCCCCTTAAAGCCACTGCCAAGACG |
| 630 | 7 | 104752143 | CGTCAATTATGGAGGCTTGGGATGTTAAAAGGCCAATATTTTTCAGTTGT |
| 631 | 6 | 26250347 | CGGCGCTTTCAACTGAAATTGTTGGTGGCTCTGAAAAGAGCCTTTGGTTT |
| 632 | 20 | 25129126 | TGGGTGTCTGGGCCGGGGCCCCTCCAAGCCCACCTGGGAGAGCACTCCCG |
| 633 | 13 | 114109420 | CGCTTCGGCCCCACAGTGCAGTTGTGCCTAAAACAGGTGAGAGTTTGAAC |
| 634 | 10 | 102899285 | TTTCTGGATTTTGCGCGGCCTGGCGCTGGGTGCCACAGCCGGTGAATGCG |
| 635 | 6 | 133562035 | CGCTACCCAGTTCCCCTGTGCAGGCCACTGCGGGCCCCCTGCCGGACTCC |
| 636 | 7 | 15725652 | CCCCAGTCGGGGTGCTGGAGCCCAAGCTGGAAGAGTTGGAGCACAGGACG |
| 637 | 2 | 66664798 | ACCGAATTCCGGGTGGAAGGACCCAGCTGTATTGACCTTATATAAGCTCG |
| 638 | 11 | 89867819 | CGGAGGCCACCCAGAGCTCACAGCCTCCTGCCAGCGCGCTCTCTGTTTCT |
| 639 | 2 | 242808479 | GGATGCTGAGCTGCGCGGGATGCTGAGCTGCGGGGGGATGCTGAGCTGCG |
| 640 | 2 | 177022646 | CGGCAGATGCCGTGACAGCGACCATTGTTCCTGGTAGACACAGGCGACCA |
| 641 | 3 | 49057259 | AATGACCAGCTTTTAATGTGACTCCTTTAACCTGGTGTGCTTTAACAACG |
| 642 | 5 | 172671526 | GCTGTTCACAATGCGCTCCGCCGGGCGGTGGAAACTTGGCTGCGGTAACG |
| 643 | 17 | 8230350 | GCCACGTCCGGGAGGATGAACGGAGCCCCGCTAAGGTCGGGAGGACTTCG |
| 644 | 3 | 185540331 | CGGCTAAAATAAATACCCTTGTGAAATCTATAGTTATATTTAAGTGCACT |
| 645 | 16 | 51147536 | TCACGGAGGCCGGTAAATATGTAAACGTGGGTTTTTCGGTTTCCGTTTCG |
| 646 | 19 | 7798327 | CGGTTCCCACCTTTTGCACTATGGACATTAGGAGTCAGATCATTCTTTGC |
| 647 | 5 | 63297615 | CGTGCTTTGGATTCAGGTTATTTCAGTTTGATTCCAGAACATTTCAGAAA |
| 648 | 7 | 2594133 | CGCTCTAGTCACAGCAGCCCACCATGCCTGCTCAGGCCTGGGAAGGGGCC |
| 649 | 3 | 170302890 | CGCGTGGCCACGCAGCCCTAACCTGGCCCTCTTGGATTAGAGGGGAGCTG |
| 650 | 11 | 66410998 | AGCCGGCTTAGGACTGCGCCCGGGATGGGAGACCAGAGCGGCTGCTATCG |
| 651 | X | 132090141 | GCCAGTAGTGATAAGCAAGCCTGGGAACCATGAGCTCTCCTTGGATGTCG |
| 652 | 6 | 26598303 | ATCTATCTTCTCACCTTGAGGTTCCAAAGATCATAACGGAAGGGGCTGCG |
| 653 | 4 | 117847088 | GTCGGTTTCAGGACTCCCTCTTTTCAAGCTGCCAGAAACTTGTCTAGTCG |
| 654 | 18 | 55095177 | TCTGTTCTACCCACGGCGGCGGAAGCGACGGGGGAACTGAAGACGAGGCG |
| 655 | 2 | 127646592 | CGGGTCTCCAGGTGTCAGAAAGCCAATCCTTACAAAGTTGAGTGATCCAA |
| 656 | 8 | 105479248 | GGCCGCCGGGCTCCGGATTTGCAGCCTGAGGCAGAGCTCGGGGGCTGTCG |
| 657 | 7 | 71800987 | CGGATCCCACCCCCACCTGGGCGCCAGACCCATCCAGCTGCTTGGAGCTC |
| 658 | 6 | 33258515 | TGCTTGTCTCCCTTGTCTCTCCTTAGTAAGCGATACGAATCCCAGCTTCG |
| 659 | 16 | 53243559 | GTAGGAAAGTTTTCAGGGCTCATTACTCGTGGAATATTGCGTTCCCCACG |
| 660 | 12 | 89744701 | CGGGGGCCTGCGGATCAGCTCTGACTCTTCCTCGGACATCGAGTCTGACC |
| 661 | 11 | 76033097 | AAAGGCGTTTTCTGCCCCCTCCGGGCCTCGGGAATATTTGTGGGCTGCCG |
| 662 | 8 | 119121281 | TGCTTTATCCTGAGCTTGGTGGACCAGTTTCCTAGTAGGTGGGGTTGCCG |
| 663 | 15 | 29396200 | GCCTCCAGCTGGGTCTGTGCGCGCGGCAGAGATGCTGGGCGTGGGAACCG |
| 664 | 4 | 13529809 | CGCCACTGCTGAGCGGTTGGAAGGGGAACACTGCAGGTACGCCCCAGTCA |
| 665 | 7 | 105319615 | GTAGTAGATGTTTCCTGGCTCCTGGATGAGGGCCCTGGCCAGTCAGAACG |
| 666 | 12 | 5540116 | CACCACGCAAGGACTCGGGAAGCGGGCAAGTGGGCAAAACTCTGCTTCCG |
| 667 | 12 | 77719408 | TGGGCATCTCGGCGTGGCCTGCGCCGGGGTCACGGGGAGGGCTGTCAGCG |
| 668 | 14 | 45429449 | AAGGGCATGATAACTGCTTAAGTATCCACGTCTCTCATTTAAGACTTTCG |
| 669 | 4 | 81186795 | CGCGCTGGATTTCAACCTCAGGAAACCCGAGAAGGGAAAATTTCCTTGAG |
| 670 | 16 | 89251975 | CGGGCCCCTCACGCCCTACTTGCACCAGACCCCTCCCACCCGGGCCAGCG |
| 671 | 15 | 51385916 | CGGGTCTACGCACCTAGGACCCGCTCGCCCTCCTCACGTGCCCACAGCTC |
| 672 | 2 | 187713986 | GTTGCCTTGGTGACGGTGGTTGGGCTCGGGGTGTCCCGGTGTGCCCTGCG |
| 673 | 17 | 49008322 | CAGGAATGGAGTGTCCCCCAGCCCTGCAATTCCGTGGAGCGCTGAACACG |
| 674 | 17 | 5019452 | CTGCCCGCGGCCCCTCCTGACTGCTAGTTGGAGCGGGGACAGCATGGGCG |
| 675 | 1 | 197889693 | TTGCTACGAATTGGGCAAGTTCTGCGCCTTGAAACTGAACTAGTGGCTCG |
| 676 | 1 | 63795711 | GCTCCGCGGCGGGTGCCTTTCACACCTGGGTTTCCTTCGTAATTAATTCG |
| 677 | 1 | 19615480 | TGGCTTCGTACCGGTGACTCGCGTCTTGGAGATTTTAACACTCCTCAACG |
| 678 | 3 | 170303540 | CGCCCTGGGCCTCCGCGCCATATTCCTGGTCCCCCGCTCCCGGTGACTCC |
| 679 | 1 | 119532189 | TCGAGACGGTTCTATCCTTTTGCAAAGAAGCCGGAAAGAGCTGGGTCCCG |
| 680 | 11 | 118842484 | CGCCTGTGAGGGTCGCTCCTCAGCCGCCGCGCTCCCACTCCGCGTCCCCA |
| 681 | 16 | 24197863 | CGGCCATGTTCTAACCTGTACCACTGGGTGGAGGGAGAGCAAATCCTTTA |
| 682 | 17 | 80545020 | CTGGTGTTGACCTAGAGGTGCCTGTTGTACTATGGTCACCGTCTGGACCG |
| 683 | 16 | 3211519 | CGCCGCCAGCCAGCCAAAGCTCTCTCTACACTGGATCCCGGCCCCGTTTC |
| 684 | 17 | 73636184 | CTGACAAACTTGTTCTGCGGGCTGCGGATGGGTGCGAGGGTGGAATCTCG |
| 685 | 18 | 31020806 | GGAGAAGGCGGAGCGGGGGCGGCCTGGAGCGCTGATCGGGCGCCGAAACG |
| 686 | 7 | 113727753 | CTGAGGTCGGGTGTCAGGGCGCGTTGCGGCCTCGCGGGTGGGCAGAGGCG |
| 687 | 5 | 170740673 | CGCTCCCGCCGTCACCTCCACCTCCAAGCCCAGCTAGCCTGGGATCTGCG |
| 688 | 3 | 27765232 | CGGCCCCGCTCTTGAATCTATCTGAGGAAACGCAGCGAAGAAACAAGCAG |
| 689 | 2 | 242702553 | CGGCGGCCTGCACCCGGAACTCACGCTGCCTCGGGTCCCAAAGGCTGAGA |
| 690 | 15 | 69136313 | GGAGGACAGGCGGCTCTGCGCTGCGGCTGTGGTGGGCTCCGTGCACACCG |
| 691 | 10 | 101841416 | GAGCAGGTCTGACATCTTGCTGGGCTTTTTCAAAGAGAGCCACTGAAACG |
| 692 | 6 | 32120863 | CGGCTTCACACCAGTCGTTCTGGTCACTTAGGCGTTCGCGTGAGCGCTCA |
| 693 | 2 | 181010312 | CGGCCAGACTGTTTTCAGAGTAGCTGATCCTTAAGTCACTGTATGCTTGC |
| 694 | 22 | 50219754 | CGCGTCATTACTGAGGTGGGGCAGAAGCAACAGTTGGTTACTCACCAGAT |
| 695 | X | 48690457 | CGCTCGCGCTCTGCTCCGCGCCCGCCTTGACCCTGCCGCCCTCGCAGCCC |
| 696 | 4 | 147561203 | CGGTGCCCATCTCGCACCCTTCCGCGTTGGCGGGCACGCACCACCACCAC |
| 697 | 1 | 47695138 | TGCAGGTTGGGGGCGGAAAGCTGTTTGCGATTCCCTGGACTGGTTGGTCG |
| 698 | 2 | 229046406 | AGGATAAGTCTGTGGTGCTAGGACCCAGCTCCCAGAGTGCCAGACTGGCG |
| 699 | 10 | 50817213 | GCTCCAGGGAGACTCACCTTCCTTCTGAGAGGGCCGCACCCCTTGAGGCG |
| 700 | 11 | 22201921 | CTCAGTATTCCTGCTGGCATTGTGACATGATTGAGCATGGCTCTTGTTCG |
| 701 | 2 | 105461368 | GCGCAGCTCTCCGTGCCGGCGTCCCGGGATTTGTATTGCATGCATGTACG |
| 702 | 4 | 174422191 | AAAGTGGGGAAACCCAGAGGCGGGAGGCTCCCAGTGGCTGGGCAAGAGCG |
| 703 | 9 | 113800909 | TCAGGGGCGCTGGGGGCGCCACACCAGGTAAGGGGTCCAGCTTGGGAGCG |
| 704 | 7 | 23287352 | GCAGTCGGGAGACTGAGGGTTAGGGCGCGGCCGCGGGGTCTCCTGGGCCG |
| 705 | 4 | 47034267 | CGCGTGTGCCCACACCTGTTTTCCCAGGCAGTCCCCTGAAAGGGGTGGTG |
| 706 | 19 | 35396475 | CGGATCCATCCTCAATGTCCAGGCAGGACTCTCACATGAAGCCGGTGCTG |
| 707 | 1 | 76540438 | GGGTCTGTGGCGGCGCAGCTTACACCGCGGTCCCCTTATTTGGATCTGCG |
| 708 | 21 | 34392851 | CGCTGTGTGGAAACTCCGGTAAAACTCTTTGGGCAACAGTCTTATCACCA |
| 709 | X | 10094247 | CGAAGTGTTCAGAATCCCCGTGCATTCCAGCGCGTTGACACTGAAGTCAC |
| 710 | 5 | 139089315 | CGGGGCCCTGTCTGGGGTCTCTGGGAGAGTCACCCCCAACTTCCTGCCCC |
| 711 | 3 | 125899547 | CGCTCCCGCCATAGTCCCACCACCGGTGAGCGCCGCCCCCACTCGGCTGA |
| 712 | 3 | 149375085 | ACTTTAAATAAAAGGCTGATCTGACCTTCTTTTTGCAGAAGATGAATCCG |
| 713 | 6 | 26250350 | CTTAAACCAAAGGCTCTTTTCAGAGCCACCAACAATTTCAGTTGAAAGCG |
| 714 | 10 | 778709 | CGCCCTGGATAGCGACTTCAGAGAAGGAGCAGAACACGTATTCAGAGATA |
| 715 | 10 | 3329765 | AATTGAGTGCGTGCACACTCAGTAAATGCGAAATGGTTTCATCACAACCG |
| 716 | 15 | 96889169 | TTGATGGCGGCCGCGAACTTGGCTGCAGCGGCAGATCTCCGACCAAATCG |
| 717 | 4 | 188916867 | CGCGGTCTAATCAGGCTACAGCCACCCATCAGCTCCGCCAGGGTTAGGGA |
| 718 | X | 139593371 | CGCGGCCCAGGAACAGCTAACGTGCCGCTTGCTGCTTGCCAGGTATGGAG |
| 719 | 14 | 38724255 | TTACTGAATTGTCTTGTTCAGGGACAAGTGGTGTCTCTCCCAGCTTCTCG |
| 720 | 3 | 64008041 | CTTTGTCACCTATCCGGCAGAGGTACTCGGCCTTTGCCATCATTGCATCG |
| 721 | 16 | 65158052 | CCAGATCATCCTATTACCATCACCATGCCTCATTAGAGTGGGGACTTTCG |
| 722 | 6 | 134217401 | CGCCTGAAGGAAGCCCGGCGGACTCTGAAGAATGCTGCTGGCAACCAGCG |
| 723 | 3 | 173303193 | CGGCCGAAGCACCAGCGATTTCTACTTGGGCTTTTCCGCTCTACATGCAG |
| 724 | 3 | 164911348 | CGGTCCAAGTCTTTCAGGCTGTCTGAGCTCTTATTCGGGACACCCAGGCA |
| 725 | 6 | 27173991 | GACGTATAAGGCAGTCCAGGCCCTTCAGGCAAAGCCCTCTTAGTTAAGCG |
| 726 | 16 | 2132315 | TCCTCAGGTCTGCCCAAGCAGCTTGTAGCTAGCACTGGGCCCCGTGCGCG |
| 727 | 3 | 38035642 | CGCGGAAGCCCCTGCCTAGCGTCTCCCCATGGCCCTTGGTACATCCTCCC |
| 728 | 10 | 119494494 | CGCTGACCCCAGCAGAGACCCTGGCTCTGAGCGAGTTCCCAGCTCTGTGA |
| 729 | 1 | 197879766 | GCCGTCCATCCATTCGATGGCCTCGCTGATGAGCATAGAAGATGAACCCG |
| 730 | 20 | 44098287 | CGCGGGCGGGCTGCCACATGAGGTCATTTCACAATCCCCCAGGATCAGGT |
| 731 | 7 | 35297699 | AGCCAGCACCCCTGAGTGTCACTGAAGTTCCTGGATGGGGTGTGAGTGCG |
| 732 | 6 | 55443868 | CGCCCCAGGCTCCGCCATGGGGAATGTGCCATCCGCGGTGAAGCACTGCC |
| 733 | 20 | 3052692 | CGGGGCCGCAGGGGAGGCACTGCGGGGACGGGCGGGGTGAGCGGGAGCCG |
| 734 | 8 | 25907218 | GGGTGACTTTCACGTGAAGCCCCTGCTGGGGGCCTTGCTGGGGTAAATCG |
| 735 | 7 | 15725477 | AAAAGCGACAGCTCAGGTAAGGGCGCGCCAGGCACCCTCGAGAGTGCGCG |
| 736 | 4 | 6247640 | CGCCGCGTCCACGATGGCTTCTTGCTCTGGAGCCCACCCTGCTTTGGCTC |
| 737 | X | 129243872 | CGCAGACCTATGATGTGGAGAACTGGATCGCCAAAATAGAGACCTCTTGG |
| 738 | X | 153061818 | CGGCCCTGCCTCATAGCACGGATCAGCTGGGGACCCATGCGCTCTTCCTG |
| 739 | 1 | 20669975 | AGGACCCGGGAAGCAGGTGGGCGGACCACGCGGGGCTGAGAGGATCACCG |
| 740 | X | 84503836 | TGGGATTTACTACAGGGGACCAAATGATACATTCAACCATATTATCAACG |
| 741 | X | 150905602 | GCCTGCCATCGCCATGGTGACAGCTTCAGAGGCTGGAACAAGCTTAGGCG |
| 742 | 6 | 28175283 | GGCAGCTTTCCTGGGATATCACACCATATAGACTGAGAATTTCAGAAACG |
| 743 | 16 | 51147902 | CGGCCCTTTGTGGCCCAGATCAAAGGACCCGAGCGCCACAAAGGCGGCCC |
| 744 | 7 | 153584582 | TCTTTCTTTATTGGTAGCGGCCAAAAAGAGTTGATTGCTATTGGGATCCG |
| 745 | 6 | 10886999 | CGTCATTTAAACGTTCCTTATTTGCGAAATTCCAAACAGATAGGCGTTCG |
| 746 | 10 | 102996310 | CGGGTGGAAGATCCCCCGCCCACCAGTATCCTGGATTTACCCAGGTCGAG |
| 747 | 12 | 132908454 | CAGTTGCCCTTTGTGTGCCTCAGTTTCCTCACATGGAACGTTAGATGACG |
| 748 | 19 | 46387962 | CGGCCCCTGCGGCTCTCTGTGGCCCACCCCCGCGAGCCCCATCCCGGAAC |
| 749 | 12 | 59287786 | CCAAAGACTGTATGGTCTGTTATCTAACTGGTAGCCTTATTTGACCAACG |
| 750 | 20 | 61472147 | AAGCAAGTGACAAGGACGCCCGAAGCACAGTGGACGGTCATGAAGGAGCG |
| 751 | 4 | 175132842 | CGCTCACACGGTGTCTACCGGATCTGGCACGGTCAAGACACAGGAACAGC |
| 752 | 6 | 100442105 | CGCTCCTAGAAAAGAGCACGACGCACCCGATGCTCGGATTGGATGAAGTG |
| 753 | 15 | 37395115 | CGCTGCGGAGCGTTTTCGACTTTGCAAAGCTGTTGATGTTGGTGGGTCCA |
| 754 | 14 | 57261601 | CTCCTCCGGTAGTCTCTCTGGACTCTCCAAGTGACTTGCTGGGGGCACCG |
| 755 | 1 | 50890678 | CGCCTGGCACGACTACTGGGCCGCGTCTTGTCAGTCTATCAGCTTCTGAG |
| 756 | X | 136633004 | CGCCATGCCCGAGCTACTCGCACTGCCTTGGTCCCATTCGGTTCCCCGGG |
| 757 | 20 | 21689759 | GAAATTTAGAGGCCAGGCCTCGCATGGGGATAATGGATGGGCACAGGACG |
| 758 | 1 | 47009707 | CGCCTGGAAACCCAGCCGCGTGGGAGAGGGCGACCCCACCTCCAACCCCC |
| 759 | 1 | 197888512 | CGGCACCAATAGCCCTGAAAATTAGGGAACAGCAGGGGCTGCCAAATATA |
| 760 | 11 | 31819464 | TGGGTGCCGTTCACCTTCCAGCCTAATCCGTGAGAAAGCGAGTGAAAGCG |
| 761 | 8 | 72275551 | CGGAATGGTGCTTTCATTTACATATTAGCAATATAAAGTTATGATTGTGT |
| 762 | 8 | 11565530 | CGGAAGCTGATGTAGGAGCAGCTGAATTCACCCCAGGGCCTGAAGGTCCG |
| 763 | 17 | 37123949 | AACAAGGGCGAGGTTCCTGGAGCCCAAACTCAGGCCTCAGCCACCCTTCG |
| 764 | 16 | 667157 | CGCCCGCGAGACCAGCCTCGGCCACTGGCCTCCCAGGGCCAAGACCCACG |
| 765 | X | 108868282 | TCTCGCTGCAGTTCATGGCTTTCGGGGAGTGACACGGCGGCTCCAGGACG |
| 766 | 19 | 14376770 | CGCCCTCGGAGGACGCATCCTAATCACCTCGGCAAAGAGCCGAGCCAGGC |
| 767 | 2 | 27958421 | CGGCCCGGCCTGGGGACACACTCCCGCGCCCTCCAGGCCCCTGAGCTGTT |
| 768 | 19 | 46916741 | CGCCCCCAGGCAGGCCGCCACCCAGCTCCAGGCTGACCTTGACGCGCCGC |
| 769 | 15 | 96959633 | GCAAAGTCTCCAAAAAGGTTACAAGGGTTCCAGCGCCTCCCCAAGTCACG |
| 770 | 4 | 25090198 | AGTCATTAACTGGCTGTCAGGTTCCTCAGATGATGGAGCTAAAAATAGCG |
| 771 | X | 118284952 | CGGCTCCCTCAGCAGCACCATGGCTAGCTTTCATGCTTGCCTCAAGATTT |
| 772 | X | 56258973 | CGGGGTACCTCGCGCCCCACGCGAGTCAGAACCTAAAGTGCTCTCTGGGC |
| 773 | 14 | 52535964 | CACATCTGGTTCTCGTTAACTTTTCTAAGGCAGCGGCCGCTGGAGCAGCG |
| 774 | 6 | 85482570 | CGCTTTGGCTGGGTTAATTTTTGTCGATTGTTCCTCATCACACTCAGATA |
| 775 | X | 73641955 | GCGCGTTGGAGGGGCGCCTCGGACCGGGAGGTATGGGGTCGGTGGCAGCG |
| 776 | 1 | 154299179 | CGCCATCAGGAGTCAGGAGTCTGGATATGGACCAGGAAAGTTGTTCTACC |
| 777 | 17 | 55888548 | TCACTGAGGTGGCGCTGAAACAGCTGACGGAGCGAAGCCTGACCCAGTCG |
| 778 | 2 | 66673316 | CTAGGCGTCGAGGCGAGGGTTGAGCCTGTTTGGCTTCAGAGAACGATGCG |
| 779 | 6 | 84140993 | CGGCCGGAGCCCTTGGCTCCATAGGGCCTCTGCCCACCATGGGCTTATGA |
| 780 | 11 | 32461240 | CGTCTGTTCTGATGCCACCGCGAGATGGTCCCCGAGCTCCCCGAGAGTCC |
| 781 | X | 136648249 | TCCTTGGACTGATAAAGTCAATCACTCACTCCTCGCACATAAACCGAGCG |
| 782 | 7 | 137028165 | AGTGGCACAGGGTGAAGGCAAACTCGATCGAAGAGCCCTCCGAGAAATCG |
| 783 | 2 | 223162881 | CGCAATTTGTCCATTCGAAATTGTCTCGAAGTTGTCTCGAAATTGTCCCG |
| 784 | 12 | 105114327 | CGGCCACCTACCACCCCTGTCCCCTGAAGATAAATGATCGCAGCTGCAAA |
| 785 | 1 | 1566699 | CCGGTCCTGATTGGGGGCGGGGGCGGGGAGCTCGGAGACCCAAAGGTTCG |
| 786 | 19 | 10715612 | AGGGTGGGCTGGCTGGGAAGGATGGCAATGCCCATTGCTGACATCCGCCG |
| 787 | 11 | 2596416 | CGGCACTCCCACTCCCACACCTGTGCAAACATCCGCCCGCTGGCCGGGAA |
| 788 | 11 | 131761763 | CCATTTTTTCACTGGGCCTAATTCTTGACAATGGTTCTGATTCTCATTCG |
| 789 | 15 | 90357992 | CGCCCCACTTGCCTGGGCCGCCGGGCGCTGGAACCTGGACCCTGGACCCT |
| 790 | 22 | 28838631 | TTTAAGTTTTTCCTTCATGATATGCCTGGAGCCCCAGGGGTGACAGCTCG |
| 791 | 3 | 138658243 | CGGGGAAAGGTTTTTTAAAACTCGGTTTCATACTATTATTATTACTAAGG |
| 792 | 17 | 79093913 | GCTCCCGGGTGGGGCTTTCCTGGAGGAATCAGAGAGCACCAGGTAGCTCG |
| 793 | 1 | 97004302 | CGGACTGGCAGTGTGCTGCTCTTCAAAGTAAGAGCTAGTGCTATAATTAT |
| 794 | 3 | 87138510 | GAGGGAACGTGTGGGTCTGCCAGGGTCCTGCAGGGGGGATGAGACCCTCG |
| 795 | 7 | 27199815 | TTGCTTTTGCGTTTTGCCTCTGGAACCATTCGACATCCGTGGCTGATGCG |
| 796 | 8 | 11540407 | CGGAGTAGTTCTAAGAGGGTAACTGTTAGATTCTGTGTCAACCTCTGAGA |
| 797 | 6 | 29855465 | GAGAATCTGAGTCCCGGTGGGTGCGTGGGGACTTTAGAACCGGGACCGCG |
| 798 | 14 | 34419546 | CGGAGTCCGTTGTATGTCGACTTCCCAGGGCCGTAAGCGAACTTTGTTTT |
| 799 | 14 | 104569519 | CGCCCCCATGCGGCCTGCAAGACACAGCCGGGCCTCCATCTCTGCAGGAC |
| 800 | 3 | 125899552 | CGCTGCGCTCCCGCCATAGTCCCACCACCGGTGAGCGCCGCCCCCACTCG |
| 801 | 14 | 51410689 | CGCCGAACTCATGTCTCCGAGGTGCCACCCGCCAGTGTCCCGCCCAAACC |
| 802 | 1 | 159506621 | AGTTAAGCCAGTTGGGCTACAGATAGCTGCAGATTTCTCTCTCAACTCCG |
| 803 | 4 | 112709384 | CGGCTCGGGCAATCCTGCGCTAAAGCCCGCTCCCCCCGCCGCCAGCAACG |
| 804 | 7 | 128531106 | CGCCCGCCACCAGGAGTACTTCTCCCCGCCCGGCGATCCCTGCCGCCGCT |
| 805 | 10 | 101282883 | CGGGAACCATGACTCCACGGCCATCCGGCCCCAGGGAGGCGTTTTTCCAA |
| 806 | 3 | 62364315 | CGCCCCTGGCATTCAGGGAATGGTTTGCAAATCTCAATGCACCACAGACT |
| 807 | 10 | 102996197 | CGTGCAGTTTATTTATTATGAGAAGCCTGACTCAGGCCTTTCCTGTGTCC |
| 808 | 6 | 44187132 | CGCCTCCCTGAGACCTGGGAGCACGTGCACTGGCTTTGAAGACTCCCGAA |
| 809 | 2 | 177025198 | AGGAAGGGCGCAGGAGGCTTGGGACCTGGGCCCCATTCCTGGGGCAGACG |
| 810 | 19 | 18385672 | GGGCCAGTCAGGGTTCTCACTGATGTCTTGGGTGTGGCTCTGAGACTGCG |
| 811 | 17 | 75315527 | AGAGAGGGCGAACGGGACGGGGACTTCCTGGAAGATTCCTACCACACACG |
| 812 | 3 | 49057661 | ACCCTGCTAAGCAGTTGTCTCCAGGTCATGCACCTTCAGAATGGAAAGCG |
| 813 | 5 | 42992774 | GGGATTCGGAGCTTCCCCAGCTGGGGTGAGCCTGGCGTTCCCGGGCGGCG |
| 814 | 12 | 115136072 | TTTCAGGGCCCAATTAATTCGCCGCGGGCGGTGGGGCGGAGGCTGTGCCG |
| 815 | 9 | 971480 | CGGTTCAGCATTAAAGTTCAAGTGGAGCGGAGGGGGCTGCATATTTTTAT |
| 816 | 4 | 175133151 | GAACTGAAGAACCGCGCAGGAAAAGGCTGCCTGGACCAGCGGGACATTCG |
| 817 | 2 | 62798365 | TGAGAGCGAGGGACACGGGTGAGTCACGCGGCAGAAACTCACGGCAAGCG |
| 818 | 5 | 59189843 | GGCGAGAGCAGGGATTGTCAGCAACTCGGGCTGCAGAAGCAGCAGCAACG |
| 819 | 1 | 111813690 | CGGAGCGACCAAGGACAGGCTCTCGCTGGCACTCAGGTTGGGTTCTCTCT |
| 820 | 3 | 16554619 | CACTTCGGAGCAGCCTGTTGGGGGTCGTAGGCTCTGCAGGCTCAGAAGCG |
| 821 | 11 | 32455025 | CGCCCAAGGCTCGGGAGAAGGGCTCTGCCCTTGTTGCCCGCCTTCACCCG |
| 822 | 1 | 110210770 | CACAGCAACCAGCACCATGCCCATGACACTGGGGTACTGGAACATCCGCG |
| 823 | 7 | 39453625 | AGGGGTTGCTGGGGTGAAGCTCGGTGGGGCAGCCCTGCCGCCTGGCTCCG |
| 824 | 5 | 131593730 | AGATGAGGCCAGCGGGGAGGCGGGTGCGTGAACTCTTGGGAGATTCTTCG |
| 825 | 6 | 31940855 | CGCCTCAGTGTAAGATGGAAACAACACTGATGTACAAGGCCTGTCATGAT |
| 826 | 5 | 86060076 | TGCAAAGCATTATCTGTGGCTGAGCCCATCACAATTCAACAAAGATTACG |
| 827 | 7 | 39015928 | CGGGCAAGTTTGGCTCAGTCCTTGTCGATGCTCCAGGCTCGGCTTCCCTG |
| 828 | 7 | 27196286 | CGCCGCAGCACGTGACCGCCCACATGACCGCCTCCGCCAATCTCAGCAGT |
| 829 | 6 | 84417933 | CGGCCCCCACATAGGGCCAGACTCACCCCCTGGGGACCCCCCTGTCAATC |
| 830 | 4 | 111532679 | CGCGGCCTTGCTGCTGCGCCTCCAAAGATCGCGCACCAGTCTGGCCCACA |
| 831 | 5 | 891207 | TTTAGAGACTCTCCTGGTGTTCACAGCTCATCGTTGTAAATGAGGGGTCG |
| 832 | 10 | 43800133 | CGCTCCTCTACAAGGAGCACTCGAAATTTTGCCGTGGGCCGCCTGCGGAG |
| 833 | 1 | 32741518 | CGATCCTGAATGAATGAATGGATGGACAAAGCGACGAAAGAACGCAGTGA |
| 834 | 3 | 169376470 | CGCTTCATCTCAAAACCGTCTGGTGCTGAGGAAGTGGCCCAAGATGGACG |
| 835 | 7 | 15727127 | CTAGAAATGCGCGTGCAATCTACACATCTCATATGACCGGGACACAATCG |
| 836 | 20 | 57090015 | TGAATGGCAGGAGCTGCACCAGGGGGCTGTGCGCGCTGCGCGGGTGTGCG |
| 837 | 10 | 102894441 | GCAGGTCTATTCCGCCGCTTGGGCAAACAGGCGGGTTAGTGCACTCCACG |
| 838 | 15 | 37390326 | GGATGGACGGAGTAGGGGTTCCCGCTTCCATGTACGGAGACCCTCACGCG |
| 839 | 22 | 17073594 | CGGACCAGCACTCCACTGTGGGTCCAAGGATGAGCTCCAAAGAGCCCAGT |
| 840 | 7 | 79083753 | CCTGCCCCGGGGAATGCGAGGCTGCTTCCCTTCTGCGTAATCAGGGTACG |
| 841 | 7 | 55589803 | AAGCTTTCTTTGGGTGAGGCAGGGCCGTCCTGCCCAAAGCCCCTCCTCCG |
| 842 | 2 | 219151444 | CGGAGCCTTAACCCTTCAAATACCCCCAGGAGAGCCCCAGCCGGGGCCTC |
| 843 | 14 | 24803679 | TGCTCGCAGTGGCCTGGGCCAGCGGCAGGGTGAGCCGAGCCTCTGCTGCG |
| 844 | 4 | 114213914 | CGGAGGGCCCCCGTCTCCAGAGAGAGCCAAAGAGTCAGGCTGACTGCTTT |
| 845 | 11 | 2293171 | ATGTAGTGTTAACGGAAGAGTTTAGGTCCTGCCTCACTGTCCGGGAAACG |
| 846 | 1 | 158259926 | GCTAGCTCTTCTTCTCCCAGGTGGTGACAATGCAGACGGTAAGAACATCG |
| 847 | 2 | 187713964 | CAAGACGACGGCGTGCAGGCTGGTTGCCTTGGTGACGGTGGTTGGGCTCG |
| 848 | 19 | 39466313 | TCCCTTTCCCACTTTGTTCTGGGAGGCAGTCGAGGGGCTCCCCGTGATCG |
| 849 | 11 | 86383182 | TGCTTCCAGGGCTTGGTTTCGCTTTGCCTGGCTCTTGGCTAGGCCTGGCG |
| 850 | 5 | 139076670 | GAGGCAGCTGGGGCGGGGGTGGCCGCGGGAGCGTCTTGCCCGTGTGATCG |
| 851 | 1 | 896763 | TCATGGCCACGAAGGCATCGTGGTAGTGCCGCTTGGAGTTGTGGGCCACG |
| 852 | 4 | 111397401 | CATTCCACCCCCCTTGTTTCCGCATTCATCCTGAGTGGCTGGTGGGAACG |
| 853 | 11 | 91957992 | TCGGCTGTGATGGGGAGCCCGGAGCAGCCAGGCGGCGAGCAGTAGCGGCG |
| 854 | 3 | 51430288 | GCTGGTACTGCTGGGGGTACCGAGTCTCCTCTGCTTTGGCAAAATCCACG |
| 855 | 11 | 115530590 | CGCAAAGCTGCTCTCTCACTCCGTGGAGTTGGATCTCTACCCCCGCAGCC |
| 856 | 3 | 121903520 | GAGGGGCACACGCCGGAGCAGCTCCAGCCTGGGAGCATTGGGCGTGAACG |
| 857 | 6 | 117086850 | GACGGGGATAGCGAGCTGAGTGTGCGAGCGCCAGGGGTTCCAGCTGCACG |
| 858 | 5 | 115152785 | ACAAAACAGGCACTCTCTGGCCTAGGAGTGGAATCCATCCTCAATTTTCG |
| 859 | 13 | 29293450 | CGGGATCCTGCTGGGGGCTACAGCCCAAACATGTTACTTTTCCAACTGTT |
| 860 | 3 | 64008007 | CGGCAGAGGTACTCGGCCTTTGCCATCATTGCATCGCGAATTTCGCTCTC |
| 861 | 11 | 128694303 | CTTGAAGCCTGCTCGGAGCTTGGGATTGTATTTTCTTTGGCTTTCTGGCG |
| 862 | 1 | 2125182 | CACAGACGGCTGCTGCTCCACCCTGGGGGCCCTGCAGGGATCAGGTGCCG |
| 863 | 7 | 116963259 | CGCCCACAGCCCAATTCCCCAGGCGCAGCGAGCGCCTGCAGCCCGAAAGC |
| 864 | 7 | 28998214 | CGCAGACTTGGGTCCCTTCCGGACGCAGCCCGCGTGCTCCCCGAGCTATT |
| 865 | X | 103813150 | AGTTCACACCGTACGGGTCGGCTCAGAGATGAAGCCAAAACCTTGGGTCG |
| 866 | 5 | 160974837 | GGATGGCGGCGGGTGGGGAGTGCGCCTGCACGTGTGTGCGCGCGGGTGCG |
| 867 | X | 101915091 | ACCGGAATTACAGCATTTCTTGGTAGAGGAAACTATCCCTCAGCCAACCG |
| 868 | 4 | 140201065 | CTGAGGCACCCTGGAAAAGTTCCCTACTTCCAAAGTCTGCAGGAGCTTCG |
| 869 | 11 | 27015808 | CGGGGAACCCCTCCTCCGGGGCCCTGGCAAGATGCTGGCTCTGTTGCCCT |
| 870 | 8 | 65290484 | CGGCCGACTGGGGTCCTGTCCTCTTGTCACCTTGTATCCCTCGCATGCTC |
| 871 | 2 | 66664535 | CGGGCATGTCTCTGGAGGAAGAAATTCAAAATCACAATAATAAAACGTGC |
| 872 | X | 13588174 | TGCAGGCACCCCCGCGTGTATGGGTGTGAGTGTGCACGTGTGCCTGTGCG |
| 873 | 5 | 158524649 | CCTTCTCCACATGCTTTTTGGTGGTGGGGGGATGTGGCTTCCCAAGGCCG |
| 874 | 15 | 97319911 | TTCTGACAGGCTTTTCCTTCATCGGTGGCCTTTGACTCCAGCTGACTTCG |
| 875 | 12 | 85307131 | TTTCATAACCTGGCGAGAATCACTGAGCGCTATGAGGCCACCGCCTGACG |
| 876 | 19 | 11531469 | CGGAGCCCCCGATCCCTCCGGGTCTAGCGCAGGCCCCACGGGGCCCCTTC |
| 877 | 22 | 37959465 | GGATCGCTTCAGAACTTCAGCAAACATTCCACGAGGACTTCTCGGTGCCG |
| 878 | 17 | 75315108 | CTGTCTTATACCAGCCCTGTGCGGCCAGGCAGGAAGGCTTCCGTGGTGCG |
| 879 | 12 | 49297846 | ATCGCTAACATGGAGAGGCAACAGGCTAGAAGAGTCCCTGTCTTGGAGCG |
| 880 | X | 43741530 | CGCATTTGTTGCTCATGGCGCTCGCCCCGTTCCAGGCCTCCCTGGTGCCC |
| 881 | 16 | 3221328 | CGCTGGATCCTCTCCACATCCAGGCCGCGTCCACTGGCTTTCCAACCTGC |
| 882 | 1 | 221064388 | CGGCTTCTTGAGAGCAGGGCTGGACTCTGCTGCATGGTTGGAGAAGCTCT |
| 883 | 4 | 41749443 | CGCGGAGACTCACTACCCCGACATCTACACTCGGGAGGAGCTGGCCCTGA |
| 884 | 7 | 138630437 | TCCTGCCCAAGACTGTGACTTCTGACCACCTGTACTTTAAAACCAGAACG |
| 885 | 10 | 102894342 | CGAGGCCGATAGCTGGGATGGGGCTGAAGAGCCCTGGCTCTGTTTTACCG |
| 886 | 10 | 134941229 | CGGCCCCAGGATGGCCCGCCCGTCTGCCCTCCGCCTTGCCCCGCCCACCC |
| 887 | 7 | 27154264 | TGAGCTGCGTGTCTGCACTCTGAAATGGGCACATGAGCCCTGGAGATCCG |
| 888 | 7 | 129253169 | TCTTACGGGGTAACCTGTCAAGAACTGTCAAATACAATTTTAGGGAGTCG |
| 889 | 17 | 78935289 | CCCTGCAGAAGCGTCCCGACGGCCACATCGTGAGTGTGAGGTGAGGAGCG |
| 890 | 2 | 214149735 | CGGAGGAACGTTAGGAGACTAGGGAAAGACCTCTGCCATTGGACTTTAAG |
| 891 | 12 | 115136279 | GGCTCTCCCCTTGGGCGGAGTGGGGCGCGGTGGGGAACACAGCTTGGACG |
| 892 | 6 | 100905458 | CGACGCCTCAGCCCGGGGAAGACTTGCACAGTACACATTTAGCGAGGATG |
| 893 | 11 | 120435056 | CAAGCGGCGCCACCGGATTCCATTTGTAAACTTATTAGTGTGCGCTCGCG |
| 894 | 1 | 197881741 | CGCACTGGCCCAAGGGGCAGAGAACATCAGAAGGAACCTGGCACATAACT |
| 895 | 10 | 73847318 | CGCCTAGAAGGCTGCTGGGACCCCAGACCCAGAGCCCCGGTCACACTCCC |
| 896 | 2 | 102803787 | CCCGCTCACTGGGGAGCAGCCAGGGCAGGCCTACCTGGCAAGGGCCAGCG |
| 897 | 2 | 228736324 | TCGTAGCCTTGGAAACGGCTAAACAGCAGCGGGACGCGGGTGCGCATGCG |
| 898 | 4 | 13536991 | CGCCATCAGCCTTCCTGGGCCTGAGCGGTCAGCTGTTTCGGAAAGTCCAG |
| 899 | 12 | 54349571 | CTGCCTGCGGCTGGAGGCGGCAGGGATTCCGGAGTTGGGGGATCCTGACG |
| 900 | 13 | 103250866 | TAAGTCTTTAAGTTATTAATACAACGACAACTATATAACTCCAGTTACCG |
| 901 | 9 | 139704416 | CGTGCTTAATGCTTGGCTGTCTGGTAAGGAAAATAAAGTGGTGCATTTTG |
| 902 | 11 | 131122594 | TGGGGTAAGTTGGTATACGGTTCAGCTAGCACACTGGCATTTTCTCTACG |
| 903 | 11 | 32915497 | CGCCTAAATCTTGGCCACCAGCGCAAACATATGTCACCCGGCCCTGGAGC |
| 904 | 6 | 31799444 | CAACATAGCGAAAACCCATCTCTACAAAAAACAATAGAATTAGCTGGCCG |
| 905 | 13 | 28502630 | CTGGCCAGTTGGCTGAGGGTGGCCGCTCAGAGTTTGAAGTACACATATCG |
| 906 | 7 | 27153655 | ACTTGGGTATGTTTCTTATGAATATTACACGCGGAGCAGCGTCTGGTCCG |
| 907 | 1 | 197898217 | CGCTTCCGGCCCCGCCCTCAGCAGACAGCGGAGCTCTCACTCCACCCGGC |
| 908 | 1 | 70710389 | CGGCGGTCACATTCTAAGTCTAGGAGTCGGCGACGATCCAAAAGCCCAAG |
| 909 | 18 | 31739521 | TGGGGTGGGGCCTGCTCTCTAGGGGACTCTCAAGCTCTTGGAAGGTCACG |
| 910 | 1 | 110210699 | GTCATGGGCATGGTGCTGGTTGCTGTGGATTCTGCAGACAGGCCTCAGCG |
| 911 | 1 | 75602412 | CGGCCAGTCTTGCCAGCACGCGAGCCCCTCCGCACTCACCAGTCCCTCTT |
| 912 | 15 | 48938576 | GCACCCTGGATTCAGGCCTGGGTGGCTTGAATTGAGCCTCAGAAGAGCCG |
| 913 | 13 | 112717207 | CGCCCCTCACTCGCTCTGGCAGCCAGAGGCCTTGTTCGAGGGCCTGGAGC |
| 914 | 3 | 183994073 | GGGACGAGGCAGAGGGGTCGGCCGCGGAGGGGCAGGCGGTGCCCGGCTCG |
| 915 | 1 | 200012526 | AGAATTTCTTTTAAATCCCTATGAAGTTAAATTCCAAAATGTCACCTTCG |
| 916 | 4 | 74734762 | GAGGTGGGATCTTGGGTGCCTGGTGATCCCAGTGTGTGGGGCTGGGAACG |
| 917 | 14 | 54422925 | CGGGGATCGCAACCTCCAGCCCGTGGGCAACGCGTTCAGCCCAAGACCCC |
| 918 | 13 | 37394589 | CGGTACTAGGAAGAGGGTTAACAGAACAGGACACTAAAAACAAAGTACTA |
| 919 | 7 | 95025611 | GGGGAAGCTCGTGGCGCTGGTCCTGCTGGGGGTCGGCCTGTCCTTAGTCG |
| 920 | 16 | 50276772 | CTGCGTTCTGGAGCTTATCAACACAGTGGATAATTAAAATCATAAACACG |
| 921 | X | 118285064 | CGCAGCCCCAGACCCACAGGCCGAAGCAATAACCAGCCAGATAAAGTTTC |
| 922 | 16 | 70563092 | CGGCTCGCCCTTTGGGATCCACAGCTAAGAACTGGCCAGGAACGATGCGA |
| 923 | 8 | 57309394 | GTAACTCCCCAAATGACTTCAGTTAACTGGGCCTTGAATCACAAGGATCG |
| 924 | 12 | 54321346 | AAATCGCCTTTGGCGTGTGCAGCTCGAGCGTTACAACGATGCAAAGTGCG |
| 925 | 14 | 95239751 | CGCAGAAGTCTATGAAAATGTGTTTGTTGAGGAGTCACTCGCCCCAGTAA |
| 926 | 11 | 132107534 | CGCACCCCACCTCCGTCTCCACAGTGTAGCCCTCAGCCTGAGATGCTGAG |
| 927 | 14 | 48145108 | GCGGCAGAGGTGCCCTGGTTGGGCGGCTGGGCGCCTGGGGTTCGACGCCG |
| 928 | 1 | 1566687 | CGGCCGCCCCGTCGAACCTTTGGGTCTCCGAGCTCCCCGCCCCCGCCCCC |
| 929 | 6 | 21667333 | CGCACAATATAAAAACGCTGCAACAATGGAAGGAAGAAAGGTTTGGGTTG |
| 930 | 1 | 241474238 | CGAGATGAAAGGATCCTGGCTAATTTCTTTGACCACTGTCTTGCAAATGT |
| 931 | 2 | 27887547 | CGAAAGTGAAGGAATCAAGTCTGGCCTATCATCTTCAGACGGCTGTACAC |
| 932 | 3 | 14852659 | CTTTTGCGAAAGGGGCGTCTTTCTTCGTCGTGGAAGGGACCCTCACTCCG |
| 933 | 2 | 201450575 | GCACGTGTGTGGGCGTCACTGGCGCCCTCTATGTGGGCGGAAATCATCCG |
| 934 | 1 | 75602167 | CGCGCTGCCCCGCACTCTGAGGGCCTTCATTAGCTCGCTCCCCGCGCCGA |
| 935 | 6 | 26088202 | CTTCCCTGAGTGCTTGCCGAGAAGGCTGAGCAAACCCACAGCAGGATCCG |
| 936 | 8 | 77595321 | ATTTCTTTTGTTACCTGTGATCCGGGCGGGGGGCTCCTTTCGGCCGCTCG |
| 937 | 16 | 51189941 | GCAGTTCAATACCAGCAGCAAAACGCAGGTTTTTGGGGGAACTCCCGCCG |
| 938 | 6 | 41339785 | CGCCCCGATGGCTGAGCCGCGGCAACACCGCGTCCTTAAGCCCCATCCTC |
| 939 | X | 119443387 | GCCCGGGGCGCTGTGGCTTGATTTGCAGGTGGTTAGGTGGTTGTCGTCCG |
| 940 | 2 | 191045309 | CGCCCCCCGCAACTACCAAGCAGCTGCCGGCGCCGCAGCGGGAACAGACC |
| 941 | 6 | 134217040 | CGCCTTTGCGCTCAGCTCCCCGCTGGCCGCGAGCTCTCCAGGCACTCCTG |
| 942 | 6 | 1378868 | CGGGGTCGCCACTGCGCCCAGCTGCAGAAAGCACTCCGCACCCCCAGCAA |
| 943 | 22 | 19754251 | GGCGGCCGCGGCGGCTGGACTCACGGGGTGGTGGTGGTGGTGCGGATGCG |
| 944 | | | |
| | GGCAAAGCGA | | |
| | AACCAAGCCC | | |
| | TGGAAGCAGA | | |
| | TCCGGTGCGG | | |
| | GTGACAGCCG | | |
| 945 | 17 | 46388390 | CGCCCCCCCAGGCTGAGCCACGTTGAGTAAATGCATGTGCACGGTCAGGT |
| 946 | 3 | 24872319 | AATTAATTTATAAATCTGCGTCCAATTAGGCAAGATGCTTTTAAATCTCG |
| 947 | 6 | 85482612 | CGCGACGCTGATTTATGGTCCAGTCGCTCGCTTGAACCCCAACGCTTTGG |
| 948 | 5 | 52327649 | CGGGATGGCCCTCCGCCTACACCGAAAAGAGAACAGACCACAGCCTTACT |
| 949 | 5 | 87975798 | ATACCACCTTGTACACACATGAACACAGAAGTGCCGGGTAACCGCGATCG |
| 950 | 3 | 147087225 | TATTCGCTGAACGTAGGGAAGTGTCCCAAAGAAACGCGGCTCCCAGTGCG |
| 951 | 16 | 49315302 | TGCTGGGGGCTGCGTGGCTGGCGGGCCCGGCCCGCGGGCAGAATGAGACG |
| 952 | 4 | 85414016 | CGCCGAGAGGTGCTGTGGAAGGGTTCACGCTCACCGCATGGGGGACCTCG |
| 953 | 11 | 86383466 | CGCGGTCCACCCATTGGCGGGAGAGGACTCACAGACCGCAGCTCCTCACC |
| 954 | 3 | 113897550 | ACCCTAAAATTTTCTTCTGATTTCTGGAGACCGAGGAGTTTACCACTTCG |
| 955 | 10 | 102894639 | CGGCCTTTGCGCAGCCGGTCCCTACCCTGGAAGGAGAAAATCAATCCGCG |
| 956 | 5 | 150323558 | ACTAAAGGTGGATACCTCCTTCACATGGTGTTTGCTATAAGGGTCAAACG |
| 957 | 5 | 115152492 | CGCGCACATCCCCGGCTTCCCCGGGCTCCGCGCCTTCCCAAGAGCCCCGT |
| 958 | 6 | 162373963 | ACGTGGGCATGTCTTGGCCCCTCTTGGGCTATTAACTTGATTGACTCACG |
| 959 | 12 | 117314052 | GACCATGTGGCAGGAAGGCTGCCTGGTTCTGCACATCTGGCCCCTTCCCG |
| 960 | 12 | 22094563 | CGCTTCCTTACGTGGCTAATAAAGGGGTGCTTACAGAGCATGGAAAAACA |
| 961 | 2 | 102867826 | CGGGGAAATGGAAGACAAAGGAAATGGGGGGACATAGCAACCCCTATTCT |
| 962 | 6 | 30095549 | GAGGGGCTGCGGCTCCGGGGCTCGGGTCGTTTTGAGTTCCTATCTTTGCG |
| 963 | 16 | 49309124 | CGCTCTTAATGAGAACACAATTACCCAGATGGAGAGGGAGCCTCACCCCT |
| 964 | 4 | 122686456 | GAGGATCTCGGCGAAGCGGAAGGCTTTGGTGCCAGCTGGGAGAGTCTGCG |
| 965 | 5 | 59189934 | CGGGCGTGCCAGCCTGGAGCTGTCCAACTTTTCAGCAGCTCCGGGATCAG |
| 966 | 6 | 29759947 | CTCACTCCCATTGAGTGTCGGGTTTCTAGAGAAGCCAATCAATGTAGCCG |
| 967 | 13 | 53313529 | CGCCCCAGGACCTGCACCCTCTACCGGCCACGGGACGTCCCTCCGCACCC |
| 968 | 7 | 100318626 | CTGAGGGACCCCGGCCAGGCGCGGAGATGGGGGTGCACGGTGAGTACTCG |
| 969 | 16 | 75680535 | CGCTCACGCAGCACTTCTGGCAGTCCCTGAAGGACCACTACCTCAAGTCT |
| 970 | 6 | 31516045 | CGGTGCAGTGGTGGGGGCTGCCCAGCATCTACATCGAGGCCTGGGTGTCG |
| 971 | 7 | 8480865 | CCGAGAGCGGTTGGGTCAGCGGCTTCATGGTGCAGCCGAGTCTCAAAGCG |
| 972 | X | 73642885 | CGCCTGGGGTCTTGCGTCCACTGGCCTGGAAAGCGAGGGCTATGCCCCCC |
| 973 | 17 | 27228511 | CGGCATTGTGGATGAGGATGTCCAACCGTGGCTCAGAGCTCAGAAAGGCA |
| 974 | 6 | 78173250 | CAGGTTTGTCCCCAGTTGATAGTTCCGTGAGTTCCTCAATTATTCCTCCG |
| 975 | 5 | 87980976 | GCTAGGGTAGAAGCCCATGAAGGGGGTCCCGCGGCGCTAGCTTTTGTACG |
| 976 | 13 | 19243756 | TCTGGATGATGTCTGTCCACCCACCGTCTCTGAGTGTTCTCCTCACCTCG |
| 977 | 3 | 14852905 | ATTAATTTAGGGCTTTTTAGTCTCATCTGGAGAGTAAGCTCCAAGCCTCG |
| 978 | 12 | 53183930 | CGGCAGCAGCGGTTTCAGCGGTGGCAGCGGCTTTGGCTCCATCTCTGGCG |
| 979 | 5 | 6636046 | CACCTTGAATGATAGAACTTGAACAGCTCATTCATACAGAGTAGAACACG |
| 980 | 12 | 108237937 | CGGTGCCCCTGACCTGCAGTCGTCACCTGCACTCCAGGCGGGCAACAACA |
| 981 | 12 | 125102346 | CGTCCCCGTTGCCATGGCGAGAGGCGTCCTCTCCTATGGAGAGTGACAGT |
| 982 | 20 | 21682505 | CTTCTTCCCATAGTTTCTTTGCAGCTAGCAGCTCCTCATTTCTCTTTTCG |
| 983 | X | 119442971 | GGGAGTAGCTCCTAAGTAGCCTAGACGCTGATTCAGGCCCGGACTGTGCG |
| 984 | 5 | 92940163 | ATCTCTCCGGCTCTTACTTGAAAAAGCACTTGGAAGAAACTGTGTGTGCG |
| 985 | 5 | 140344290 | AACATCCCTACAATAATTCTGTCTCAGTTTGCTGTGGTAATTTGATCTCG |
| 986 | X | 151143125 | TGGAAGAACTTTGGACAACATTTCCGCGGAGACCGGCGCGACCACCTGCG |
| 987 | 2 | 37571732 | CCCCGGGGTTTGCGAAGGTCCGGGGTAGGAGGGCTGCAGCCTGCGCGACG |
| 988 | 16 | 1017338 | CGACCCTGGGGGTTCCTGCCATAAAACAGTGAGGGATTCTGGCCAGGCTG |
| 989 | 3 | 147110367 | GGGCGCTTTAAACAAATGGCTGCCTCCCAGCGCTCTGAGTTAAGGGACCG |
| 990 | X | 118407645 | CGCCCTGAACTTGCAGGCAAGACCAGTGGCAATGATGGACTCTTCCCGCC |
| 991 | 8 | 38965014 | CAGTTCTTAAGGTCCAGCAACACAAAGCGAAAAGTTAGTGGAGGACTACG |
| 992 | 16 | 66461802 | CGGAGTTTGGAGTTTGAACCCACCCGCACCCCTTTGAGCTAGGAAGTTTG |
| 993 | 7 | 139208852 | TGCTGCGGCGATCCGGGTCGGGCTACGAGGGCAGCACCAGCTGGAAGGCG |
| 994 | 3 | 119180915 | CGGGCATGTCCAGGAACCAGTCTGCTTCCAGTGCCACCTGTAGTTGCAAC |
| 995 | 6 | 33169606 | CGCTCTCTACTTCAGATCTTGCTCAGTTTTGCTTCCGGTGGGCTCCTGGG |
| 996 | 1 | 65730351 | CGGTTCCATGCCATGTTTTGGAATTTGTGTTTACGTCTGCCTTCATCCCC |
| 997 | 11 | 71955547 | CGGCGCTCGTGTGAAGAAGTTAATTCAATTCGCTCTGATAACCGAGTTAT |
| 998 | 1 | 197888880 | TTTTAAGCGAGGCTCCGGATGGCTGGGATGTCTTTAGGTTAGTAGTCCCG |
| 999 | 15 | 31505024 | CGAACCTTTCAAAACAGAACAGGCGTTTGGTGCCAAACAGGGCAGGGTGT |
| 1000 | 7 | 127256676 | ACCAGGCATTGAGGCTCTGGTCCTACACACTGGGCATAGAGATGAACACG |
| 1001 | 22 | 19743029 | GGGATGGAGGGACGATTGGGGAGCGCCCGAGGCTGCTGTGTTGGCGCCCG |
| 1002 | 5 | 154027129 | CAGGCACAGTGTGGGAAGAGTGCCATTCAATCCTCCGCGTAACCCTGGCG |
| 1003 | 1 | 52832660 | CGGCTTGCAGAGTCCAGTTAACAAGAGATTGAGCTCAGGAGTCCGAGACC |
| 1004 | 7 | 95026097 | CGCGTCTCGGGAGCAATTCAGTGTCGCCTCACGTCTGGCCTTGCAGAAGT |
| 1005 | 21 | 42798747 | GATCCTGCTGAATAAAGGGGGTTCCGAGCTGGGCCGAGATGGGGCATGCG |
| 1006 | 11 | 5664261 | CGAACTTGGGAAGTGACTGAAAAACTTGCACTGATGAATTGGAGGGGTAA |
| 1007 | 1 | 173834077 | TTATCATCATTGTATCGGCTTTACAACACTTAAAAACCAGACTTGAGGCG |
| 1008 | 2 | 201450610 | CGCCCACACACGTGCTGGTGTCCCGGGAAGAGTTCCTGGCAAAGAGCTCA |
| 1009 | 4 | 1535673 | CGCGTCTGCGATGGGAGCTCGATAAGGTCACCGTCAACAGCATCCACACG |
| 1010 | 5 | 140420502 | TGTGCAAGCCGAGGTATTGGCCGCCATAACGCACACTGACTTTTGTACCG |
| 1011 | 12 | 49297901 | CGAGGATATCGCTAACATGGAGAGGCAACAGGCTAGAAGAGTCCCTGTCT |
| 1012 | 6 | 146350131 | CGGCGCAGAGCCCCGCCAACTCGCCCGCTCCTCCAACCTTAAGTCGTTTC |
| 1013 | 11 | 44327267 | TATTTAACCACTGAGTCTAATTCGAAAAATCGGGACTGGGCCCCTAGGCG |
| 1014 | 4 | 65778481 | CGCCAAACTTTCAATAGTTTGGAGTGTAGATGTGAGACTTCAGCTGCCCT |
| 1015 | 7 | 27196320 | CGCCAATCTCAGCAGTCCTCACAGGTGGTCTCGCTCCGCAGGGCCCGCAG |
| 1016 | 2 | 162283434 | CGCCGTCTAGTGGCGGAATTGTCCAGGGAAGGCAAGTTAGTTTTATCCTC |
| 1017 | X | 139583634 | TGCAGGTTTGCTCCAATCACAGCAGCAAGGATTGAAGCACCAACCAGCCG |
| 1018 | 16 | 87417818 | CGCCTTACAGCCTCCAAATACAGAGGCAGCAGCCAGAACCCCAAAGGGCG |
| 1019 | 1 | 45285821 | ACACAGATGCGTGTAAGAGGCATCATGCTCTAAAACAGTGCAGAAATGCG |
| 1020 | 7 | 157932951 | CGGAGTTGTCACTGTGCCACTTCCTGGACGCGGTGAGGAGGCTTCAGCTC |
| 1021 | 13 | 36919344 | CGGGATGCTACGGAGGGAGGTGTATACAGGTGTTAGACAAAGCCCCCTCC |
| 1022 | 1 | 197889163 | CGGACAGGTACTATCTGCTGGCTGTGGACAAACAGTGGCATCTGAGATGC |
| 1023 | 7 | 25891005 | CGCAAAACGAACCCGATATTTGCAAAACTTAGGAAAAGACACACCGGCGT |
| 1024 | X | 119133965 | CGGTCAGTGCCTGGCTGGGGACCCGGACCGGTGATTTGTTCAACTCTAGG |
| 1025 | 12 | 2046045 | CGCACCTTTACACCACGCCGCGCTGCCCCAGCCTGCGTGCTGCAAACGAC |
| 1026 | 3 | 160122503 | ACGTGCTGCTAGAGTGGAACAAGTATGTCAGTTCATCCAAACATAAAACG |
| 1027 | 2 | 21022565 | AGACTCCCTACAGGTGGGCTACACTTAGGGGTTCAAATGCTGCATTACCG |
| 1028 | 6 | 81233028 | CGGCCGTTGAGCTCATTTTCAAAGAAAAAGGAAGGAGGACACTGGAAAAT |
| 1029 | 20 | 11667735 | TTTGGGAGAGCAGCTGCGAGGGGACAGAATGCTTCGCTGTGGTGGTTCCG |
| 1030 | 6 | 28457624 | CAAAGCCTGCGGGCGGGGCTCCTTGCAGGTCCCCGAAAGAATGAGAACCG |
| 1031 | 8 | 4849522 | CGCACAGCACCTCTCCAGCTCCATGCACGCCAGGGGCCTGGGACCCTGGG |
| 1032 | 12 | 75874499 | CGGGTTTCTCCATATATGGTGTGCAGCTCACTTTGTGTTCTTAACCCTGT |
| 1033 | 2 | 190044504 | CGCTTGAAAAAAGTTTTCGCAGACTGTGCTGGAGCTGGTGCTGAAAAAGG |
| 1034 | 14 | 54261853 | CGTCTTAAAACTTTGCACTTTAAGCTGGGCATAGTGATGCATGCCTGTAG |
| 1035 | 14 | 54421578 | GTATGTGCGTGGAGAGTGGGGCGGCAGGCAACCCGAGTTCTTGAGCTCCG |
| 1036 | 1 | 5334430 | CGCTGACTCCTCTTACTGGTTCTTGGGTAGCAGCCCCCAACTTGGTCTGA |
| 1037 | 6 | 108485152 | TTTCCTGTCACCAGTCCCCGATCAAGCATGGTGTCAAGAAGATGCCTACG |
| 1038 | 14 | 74179101 | GCATTTTAGCCAGCAAGACCCGCTTGCATTGCAGTCGTTCACGGTGCTCG |
| 1039 | 1 | 119529266 | GGTGCTCCTGCTGAACAGCCAGAGCTGGGGAGTAGACCCCGCTTCACACG |
| 1040 | 1 | 50896251 | CGCCAGCCCTCTCTGGGCTCGCCTCCCGGCTACCCCGGAGTCAGACTGCC |
| 1041 | 13 | 80657784 | CGGCCCGCTAAAAGCATGGACTGTTTTGGCAGTCACTGTCAGACCTCCCG |
| 1042 | 1 | 91316253 | CGGCGGTTCTTAGCTTTTTAGTCTCTGTCACGCTAATACATGCTGTTAGT |
| 1043 | 4 | 23791473 | CGACTGAAAAGCCATAACATTGTTTCATGACTAGGATGTAGGCTAAAGGC |
| 1044 | 6 | 78176085 | CGGAGAAAAAACAAAGCAAAATTTGTTCCCCACACGGTTTTTGGCTTTAG |
| 1045 | 12 | 124397892 | AAGAATCAACATGGCGAGAAACTCTGTGGGTGACGTCCGTTTACAAACCG |
| 1046 | 6 | 100442118 | CCGAGCATCGGGTGCGTCGTGCTCTTTTCTAGGAGCGTGGGGTGCCTTCG |
| 1047 | X | 63615383 | GCAAGATGGCGGGAACGGCTACGGTCAAAGTGTCGCGATAGGAGCTTTCG |
| 1048 | 6 | 27513009 | CTTCGATGACATTCAACTGACTTACTCTTCCGAAGGAACGGCTAAAACCG |
| 1049 | 19 | 54369447 | GAGGCCGAGTCCAGGAGTGGAAGCCGCGCGAGTGGCGAAGGGAGCAGTCG |
| 1050 | 6 | 146755301 | CGGCGACCACTCCGCCTCTGCCGTCCCACCTGACCGCAGAGGAGACCCCC |
| 1051 | 8 | 105479383 | TCAACTCAGGGCGCAGAACACTTGGGGTCTGAGTGTGCAAGGACCATGCG |
| 1052 | 1 | 215256254 | CACACAAAGACATGCGAAGAGGGGCTGAACGAGGCTCGCGCACAAAGACG |
| 1053 | X | 37004582 | CGGGACACTGATCTGGGACGCTGGGACTCAGCATACACACACCTGGCGGT |
| 1054 | 2 | 202122309 | CGGGTTACAACTAAACTGGAGAACATGGCCAAGGATGGGAACTCAGCCTG |
| 1055 | 13 | 112717244 | CGAGTGAGGGGCGCAGAGGCGGCAGAGAGCGGAGAGCCCCGGTGTCTCCG |
| 1056 | 17 | 78935269 | AGCCTGGGTGGTGAAGGCCTCCCTGCAGAAGCGTCCCGACGGCCACATCG |
| 1057 | 6 | 89745356 | GTAATAATTATTCCTGACAGTGACCAGTCACTGAACATTCCAGGCTTACG |
| 1058 | 1 | 224372558 | CGGTCCCCGTTTCCTCAACAGAGGTTAAAAAGCTCGTGCTCTGGCCGGGC |
| 1059 | 8 | 87081978 | CGACCCCCCAGTGGGGTTTCTTTCCGAGGGTCCCGCCCCTCGCAGCTGCT |
| 1060 | 6 | 3978604 | CGCTGAGCTTTTTTGGTCAACTCTGGGATGTGTGCACCATCTAATCGGTC |
| 1061 | 7 | 150710730 | CGTCCAGGGCCAGCCAGCAGCCCCGGGCTGCGCCCCCGCGCCCACCCCCA |
| 1062 | 2 | 100722408 | GAGTCCCTTCCAGCACTCTCCCCTTTGTTCCTTTTTCTGGGCAAGAACCG |
| 1063 | 11 | 31826574 | TGTTTTCCTATTAGTTGCTTCGCGGTCGAGTTCTAGGCGCATAATCATCG |
| 1064 | 8 | 143580965 | CGCTCCCACCGCGGCACTCCGCCTCTGGGCCTTTGAGGCGCCTGGGGCCT |
| 1065 | 7 | 601503 | GGACGTCGGCGCCAGGGCTTGGGGTGGGAACAGGTGCGGGGCTGCGTCCG |
| 1066 | 6 | 146350585 | GAGGGGCACCACTCCGGGAGAGGCGGCGCTGGGCGTCTTGGGGGTGCGCG |
| 1067 | 6 | 27526256 | CCAGCGGTACTTGCTCCGTTAGGGGCGGTCAATTAAGTGGCTTCTTGTCG |
| 1068 | 11 | 6890860 | TTCCTCTGCCTGAGGTGCTGACCTTGCCTCTTTCCCTGACAGTAAGAACG |
| 1069 | 6 | 150461119 | CTGCCCTCTCATAGCAGGGAGTAATTGCCTTCAGGTAAAAGTTGATTCCG |
| 1070 | 16 | 14402157 | CGCAGCCTGGGATATCACACTGGGCCATCTCAGAGAGGAGAGGCCTCTGT |
| 1071 | X | 144899592 | CGGCTCACTGGCCGCGTCGCCACCGCGTCCCTGAGTGCTCAGAACTTACC |
| 1072 | 12 | 116946163 | CGCCCGGGCAGCCTGGGAGGGGCCCTTTGACCTTGACCCCGCTCCTTGAA |
| 1073 | 6 | 31696229 | TCCAAATGGACCAATCACCGAGGAGCTGAGATCGTGGCGGACACGTTCCG |
| 1074 | 17 | 1957365 | CGCCTCCGCCCCGCACCTCCTGACCCCCGCCCACACACGGCGTGGAGCGG |
| 1075 | 2 | 131101440 | TGCAGGAGTTCCTCTGAGCTCCGTGGGGAACAGTGCTGGCCCCGAGTGCG |
| 1076 | 11 | 86383585 | AGTCTCAGGCGGGCATTCTGAGCTGCTTCTTGTCACTGGGGAAAACCACG |
| 1077 | 12 | 22094479 | TGAAACTTGGAATATGATTCCATGATTCTCCGGAGTCGGGTTTTCAAACG |
| 1078 | 12 | 52627047 | CGCCCGCCGCTAGGTCCATCCCGGCCCAGCCACCATGTCCATCCACTTCA |
| 1079 | 7 | 19548878 | CGCCAGTAGTGTTGTGGATTGGGGAGGAAAGTGAACAGTAGAGAAACAAA |
| 1080 | 2 | 114031854 | TTTTCATGGACCCCAGATAAAGACTTTGACAGCCAGATATCCAAGATCCG |
| 1081 | 6 | 32223336 | TCTCCCAGAACAACAGTTGTGATATGATCTGTTTTGGGGCCCTTCCTGCG |
| 1082 | 2 | 201450601 | CGCCCTCTATGTGGGCGGAAATCATCCGGCGAAGATGAAGACGACCCCTT |
| 1083 | 8 | 72756155 | CAAGAGGAAGCGGCCCCGTGTGGCTGGGGGCGGCGGCGCAGGTGGTAGCG |
| 1084 | 4 | 154680808 | CGGACGCATGACCTTCTGGAAGATGCCACCGTGCGGGGTGCCAGTCCCTC |
| 1085 | 22 | 19749961 | CGGCCACTACTGCTGTGCCATCATGTGGGGGTAGCCCATGGCCTACACCC |
| 1086 | 1 | 241198066 | CGAGGCCATAGGAAGTGAGAGCGCCATCATACTAATGTTAAATAGACTGA |
| 1087 | 19 | 46800479 | CTGCAGTCCCTAAGGGTGGAGAGTGCGGTGGGGATGTGCCTCCAACTGCG |
| 1088 | 10 | 45914949 | GGGCAGGCAGAGGCCTAGGGGGTGGGCTCTGAGGGCAGCCACTCCCACCG |
| 1089 | 6 | 10882238 | GAAGCTGGGGACAATGGTTATGGACCCGGGCGGGGCCTTGTCCTTGGCCG |
| 1090 | 2 | 37571687 | CGGGGCTGGGCGGCCCTGGGTGACAGACACAAGCTTCTGAACCCAAGCTA |
| 1091 | 16 | 66612955 | CACATTTAGGCTGGAATGGGTGACAGCATGCGAGCTCGGGGGTGGGCTCG |
| 1092 | 3 | 35706038 | CCTGCAACTTCAAGTCCCCAGGTGGCTCTGCCAGAGCGTTCACGATGACG |
| 1093 | 1 | 87800511 | CGGCTGCCTTGCTGCAGCGAATCCGCACAATTAAAAGCTTTAATTAGTGG |
| 1094 | 5 | 87974464 | CAGGTTACCGGCCAGAGTGGTGGCTCTTGGTGCAGGGCATGCTGCTCTCG |
| 1095 | 17 | 75315244 | CGGGTTTGAATGAACCCTTGAGGTGAAGCTTAAAGACAGAGAGGATGCCC |
| 1096 | 16 | 49311782 | CGCTCCGGCTCCCGACCTCCCCGCTGCCGAAGCAACTGGGTCAGCCTTTC |
| 1097 | 12 | 54391325 | CGGGCTGCACAATGCCACGGCTGCTCCGCCAGCTCGCCCCTGCCCCGTCC |
| 1098 | 8 | 145728526 | CGGCCCCCAGGCCTCTCACCGTGCACGTCTCTGTTCCGCTGCCACAACAG |
| 1099 | 8 | 109094032 | CGGTGGAGGCTTCTGTTCTCAATGAAATTCACTTGCGGCTTTTTAACTGC |
| 1100 | 1 | 29139522 | GGACGTCGGAAACATGCCCATGGGGTGGGCAGAGCTGCTCTGAGGTGCCG |
| 1101 | 6 | 28415023 | GCATGCCCACGGCGACAGCGGCCACGTTAAGTGAGGGTCGTCTTGTTCCG |
| 1102 | 2 | 231712971 | CGGCCCGCGACGGAGTTCCCAGGAAGAAGGCTGGCTGCAAAAACATTCCC |
| 1103 | 16 | 1730579 | CGGGCGCCTTGTAAGCGTCACATTTAGTTTTGCTGTGAAGCGAGTCAGGT |
| 1104 | 10 | 70482290 | CGCCATGGGCTACTCAGTTTACAGCCACTGCAGTATCACAGCCAGGTCAG |
| 1105 | 13 | 25562840 | GTAAGACCTAAAGTTCTTAGGAGATTGTCCAAAACAAAGAAACATGTCCG |
| 1106 | 14 | 57274739 | CGCCCGAGGTCCCGCTCCACTGCCCGCGCCCCCCGCGCAGCCTTATATCT |
| 1107 | 17 | 38821420 | TGGCTAACTGAACCTTATCGATAGGATGAGTCGCTGCGGCAGTCTGCTCG |
| 1108 | 21 | 42688849 | CGCCCCGAGGCGGGGGCGCACTCACCACCAGCCAATGGGCGCATCTTCCA |
| 1109 | 2 | 239974554 | TGCGGGACCCGCCAGAGTGTGCTTGGCTTCCGCGTGTCCGTGTGTCTGCG |
| 1110 | 10 | 45914688 | CGCCCGGCCTCCCTGCCTCTGCGCCTTCACCTGACCGCAAAGGCGGCCAG |
| 1111 | 1 | 35258594 | GAGGTGGGGACGATGGCCGGAGTGACGCCCGCGGGAGCCCTGCTGGACCG |
| 1112 | 3 | 40657083 | TCCAGAAAGGGTTAAACCCGGGCAAGCACCAGCTCCACGCAGTTAATCCG |
| 1113 | 11 | 134147327 | CCCTCCCCAGGGATGTAGCTCTCAAGCAAACCTAGGCTGGGACGGATGCG |
| 1114 | 11 | 127332027 | CGGGGATCTATTGTGTCCCATTGAGGACTTAAATGAGCTGAATAATTGGC |
| 1115 | 12 | 16757985 | CCTACACCGCCGAGGACCGACTATTGTGAAGCCACTTTGGGAGCGGGTCG |
| 1116 | 11 | 86383464 | GGGGTGAGGAGCTGCGGTCTGTGAGTCCTCTCCCGCCAATGGGTGGACCG |
| 1117 | 1 | 116336029 | TACCAGCTGAGCCTGATTACACAGTCTGTGTGCCTGTCCCAGGACAGACG |
| 1118 | 6 | 123317487 | CGCCCCGCTGCGCCCACCTCCCCGGCTGCTCCCGGAGGGCTCACAAAGGC |
| 1119 | 6 | 6007523 | GCAGGTTGAAACAATCGAGTGGGCGCGCCAGGACTCCCCAGCTCCTGGCG |
| 1120 | 12 | 89744621 | CGGACATCGAGTCTGACCTTGACCGAGACCCCAATAGTGCAACAGACTCG |
| 1121 | 11 | 32421808 | GCTTGTCTGAGCGAGACCTAGACCGTGACGGTGCACGCCTACGAGAATCG |
| 1122 | 1 | 63795934 | CGGTTTAATTAAGCCACAGCCCTAGCACGAGGACCCCGGCGACCCATCCG |
| 1123 | 6 | 110679107 | CGGGAGGGGGCTTCTGGTCCCAGGCCCAAGACCGACCGCTTTGCAACTGA |
| 1124 | 3 | 181413847 | CGCCCCCTCGGACCTGGCGCCGCTGCGCCTGCTGGAGCCTCGCTTCCCCA |
| 1125 | 16 | 51147461 | CGCTGCAGATTAATTTTGTCATAAAATTGACTGGTGTGCTAAGGGACGTT |
| 1126 | 6 | 31516015 | CGGCTGGTCCGGGCCCAGGCCCTCCTCCAGCGACACCCAGGCCTCGATGT |
| 1127 | 10 | 123313831 | GCCTCTCTTTAAATATCAAGCTCACAAATATGTTTAATGAACTTGATACG |
| 1128 | 3 | 271185 | CGCATTCTTTTGGTTTGAGGAATGTAGTATTGTCAGGTTGTTGCCTGCAC |
| 1129 | 8 | 105479058 | CGCCGTCCTCCACCAGCACGTCGGCCACCTCCGAGAAGTCATCGTTGACC |
| 1130 | 11 | 268976 | CGGCCAACACAGGAGGCACTGACCACGGGGCAGGCGCTATTTAAAAATCG |
| 1131 | 1 | 2125114 | TGGCCTACTCACCTGGGGGCGAACTCCTTCTGGCACATGGGGCAGCTGCG |
| 1132 | 7 | 64343315 | CGCAACACTCCCGCAGAAACACACAGCCCGGCAGCTTCTGAGGCAGCGCG |
| 1133 | X | 118601891 | ATTTGAAATACAGACAGGATCCCAACCTGCACTTGCATGACCTTGTCTCG |
| 1134 | 14 | 42075406 | AAGGGAGCCCAACAGTCTTCATAAGCCAGACCTAGTCTCTAAGGTCACCG |
| 1135 | 2 | 209536324 | GGGAGAGCATTTGCCCTTCTGTTTTTCTCCCTTTGTTACTGATTGGTTCG |
| 1136 | 12 | 66122804 | CGCCCAGACACCTCTCGAGGAAGGGAGATTGGACTTTCCCAGATTGGAGG |
| 1137 | 10 | 22624374 | GCCACCAAATGGCGGCTCCGGCAGCCTGCTGCACTGATAAAGAGGACTCG |
| 1138 | 1 | 50892511 | CGGCCTCACCCCAACGCAGTCCCTTTGGACGCCACCAGTTCCTGGGCGTG |
| 1139 | 10 | 101290699 | CGGAATTTTTGTGCCCGAGCGCTCGGGATCCACGGCAAAACCAGGCACAT |
| 1140 | 12 | 6604190 | CGAAGACAGGTCTTGAATATAGACCCTGACTTTTTCACTGTTTGGTTTCT |
| 1141 | 6 | 87862264 | ACAGATGTGCTTGCATCCGGGCCGTGCGTGCCTGCTTCACGGGTTTTCCG |
| 1142 | 6 | 32977217 | CGCCCTCGCACTCACCCTTGGTGGCCCCTGCCTCCTGCGGGCTCAGGAGG |
| 1143 | 7 | 157185402 | TGGCTCTCGGGTGTCAAAGGCTCTCCCAAATCTACATGGGGTGTTCCTCG |
| 1144 | 4 | 85418371 | GTGTCTCGTCCGAGGCCCTGGCTGTTCCTGAAACGGCCTGGCCAGGACCG |
| 1145 | 5 | 140797280 | CGGCTGCGCAGACCTTGCCCAGCACACCAGATTGCCAGCTCCGAGACCCG |
| 1146 | 11 | 5809911 | CTCCTTCCCACTGTGTACCATGTACAGCATTGCTATTACAGGGAACTTCG |
| 1147 | 7 | 8481537 | TAATTACCTCCTCTGCAAAGAGCTTTCCCTAGTATATTTTATTGATTGCG |
| 1148 | 6 | 121758653 | CGCCCCGCCTCATTCTCGGGAAGTTTCAGGAACTTCCAGGGGAGTTGCAA |
| 1149 | 3 | 16216215 | GCTGGTGGGTTCTCAGTCAAGCAGGGCTGACAGCCGTCCCTCTTTGGACG |
| 1150 | 5 | 149669368 | GGCGTTTTGGAGGTGGTTGCCATGGGGACCTGGATGCTGACGAAGGCTCG |
| 1151 | 11 | 126173246 | CGAAACCCTGGGAGACGAATCATACTGCAAATCATATCCCTGGGTAGCAG |
| 1152 | 12 | 110044639 | CGATTCAATTAGGCCGCCCCGCTCGGAAGCTTGTCTCCTCTGATATCCAG |
| 1153 | 1 | 50884480 | CGGCTTCCGCCCACCCATGGACTACGCCTTTAGCGATCTCATGCGTGACC |
| 1154 | 1 | 200003800 | CGCGCCACGCCAGATCGAGATACTCTCATAGTGATTGTGAATCAATCGCC |
| 1155 | 5 | 140235586 | ATCTTATCCTTCTCTTGTTTTACAAATCGACGAATCAGCGGTCTAGAACG |
| 1156 | 7 | 154002064 | CGCCCGCAGCCACAGCCTCGGTCACCGCATCCATCAGGGCCCCGCGCCGC |
| 1157 | 13 | 36919409 | CGGCTTCACCTCTGGCACTCTCTGAAACGTCGTTGTCTATGGTTTCCCTA |
| 1158 | 5 | 156695410 | CGGTAAAAGGCCCTGGTAGAACACTACATGACCCCTCTGATAAATCATGC |
| 1159 | 2 | 32583696 | CGTAGACGAACAAGGAAAAGTTTACTATGTGAGTAATAAGGCATTTGTAA |
| 1160 | X | 106750120 | GGGGCTGGGTTTCTAAGGCAGGTTTCTAAGGTGTCTTCCTACAGACACCG |
| 1161 | 17 | 37123767 | CCTTCCCGCTAGGGCTTCTGGGAAGATCTACAATATTCATGAGGGAGGCG |
| 1162 | 5 | 14154788 | ATGCTTGCTCAGGTTTATGCCCACATAAAACTTCTGAGCTGTTGTTTTCG |
| 1163 | 11 | 111385450 | GAGGGCAGCCGGCAGGGGCTGGTACCAACTTGGCGGTTCTCGGAGGGGCG |
| 1164 | 8 | 65498537 | CGGGTAACTAGAGAGCTCTATCGAAGATGATCATTTGCTGGTCCTGCAGT |
| 1165 | 1 | 47696701 | TTCCCGCTGCACTCCGGCTTCATCCATTTCTGGTCAGTTTCGTGGGTGCG |
| 1166 | 12 | 33592642 | CGCCCTCCGCTCCCGCCATGGGGCCAACGTGGGGCTCCTAAGAGTTGCCG |
| 1167 | 19 | 49617042 | CGGCAGGATAGTTTATGCCTGGGCTCCTGCGTTGGCATATGCGCCTTGAG |
| 1168 | 15 | 25486849 | CCTTTCCTGGTGCCCCAGTCTTCTGCACTAAGCTGTGGTGAGCACATCCG |
| 1169 | 2 | 20871002 | CGAGCCCACCAACCATGCCATCATTCAGACGCTGCTCAACTCCATGGCAC |
| 1170 | 12 | 50444522 | GAGGTGCTGGGGAGCAGAAGCTGGGATCTTTCCGATTTCAGACTCCAGCG |
| 1171 | 4 | 111532996 | GCCTGTCTCCCTTTACCCTGACGGTATTTTCAGAGGCACTAGGAGAAGCG |
| 1172 | 1 | 119532116 | TTGCAAAAGGATAGAACCGTCTCGACCAGGGCACTAGGACTGGAAGATCG |
| 1173 | X | 119134323 | CACGGCAGGAAGGTGGCGGTAAAGTCAGGGGCGAGCTCCTGGTCAGGGCG |
| 1174 | 7 | 157314735 | AATAGGGGGTGAGGACATTCAGATGCCCAGGGACCACTCGGCCCGTCACG |
| 1175 | 6 | 100906685 | TACTCTTAAAGTGCTAAAACAGTGTCCACTGCAGAGTAAACTTCCTTCCG |
| 1176 | 3 | 184243718 | CGCGCCCAGCTCCCTGTGAATACCCACTGATTGCCCCCGGAGTCTCCCCG |
| 1177 | 4 | 81185022 | TGAGATGTCCCCAGCAGGGGGCTTTGGGCGATTAACTTTTCGGAAAGTCG |
| 1178 | 17 | 41974087 | GATGGAGTCACCCCCACCCTGGGAAATCGGGCACCTGACATGGACAGTCG |
| 1179 | 1 | 50889442 | CGCAGAGAGGCGCACCGGACACGGTAATTAGGAGCAATTCACACGCTCTC |
| 1180 | 11 | 69452398 | CGGCTGGTACCCTGAGCAATATGAACCCCCAAATCCACCAGAGATTCCCT |
| 1181 | 4 | 85403409 | GGCCGGACTTGACCATTTTTAAAATGTTATGAATGACACATTTAGAGACG |
| 1182 | X | 119125722 | TTGCTCTGGAGCTTCCCAGTTCCCAGGACTCTGGGGCTCTTCCCTGGACG |
| 1183 | 6 | 27725188 | GACGCAGCGCCTTTGTGTGGCGGCGGGTGTCGGGGGCGGGAAGAATCGCG |
| 1184 | X | 70150722 | CGGCCCCGTTGATGCAACTGCATGCCATGGTTGCCAGAGAATCCTAGGTA |
| 1185 | 6 | 27647713 | CGCCCTCCGAGTGTATGTCTATCCCAGAAGAAAACCTGTCTTAAAGTTCG |
| 1186 | 8 | 49468988 | CGCGGCCGACCCCCTGTCCCCACCCGAGCAGGATTTCCTGATGTAAACAC |
| 1187 | 2 | 177004604 | CGCATGTGCAGTGAAATGGCCTAGCCCGGGAATTCGATCGGCAAAGCACG |
| 1188 | 10 | 135342218 | CGGCGATGGCCAAATAATAAACTAACAGTAATATTATAGTAACAGCATCC |
| 1189 | 7 | 100318194 | GGACGCCTGGGTTGAGGCTCGCCGGGGCGTGAGCGGGGCTGCTGCAGACG |
| 1190 | 4 | 7637533 | AGCACACACCTTCGTCCGCATTTCAGGGACAAAAGTCGTCCAAGGAATCG |
| 1191 | 1 | 2938799 | CGTGGCGGGCAGGGACATCACGGAGCTCCTCATGCAGCTGCTCCTGGCCA |
| 1192 | 6 | 85478296 | GCCCATTTGTGCGGCGCTGCCTGGTGACTCAGCGTAGGCTGATGTTCACG |
| 1193 | 17 | 33700817 | CCTAGAAGCTCCCACCCTTCTGACTGTTGCCACGTAGTTCTTAGTTTCCG |
| 1194 | 12 | 120242330 | GTGCTGATCCCAGTGACAGAGCAGCATGAGTCACTGCCCGCCAGGGCTCG |
| 1195 | 6 | 84140683 | CGGCGCCAGCCATGGAGCCCGAAGCCCCCCGTCGCCGCCACACCCATCAG |
| 1196 | 1 | 110230633 | CGGGCAGCCCTGAAGAGGAACGGTCCCTAGAGTCCGTCCCGCAGCTGCTT |
| 1197 | 1 | 153509096 | TGTGCATGCACGTGGGAGCCCAGCCCTGTAAGCACCATTCGTTATTTACG |
| 1198 | 14 | 51891409 | CGCTGTTTCAGACAACAACAAAGCCACAGGGAGGAGTTTAGCTGCAGTGA |
| 1199 | 14 | 101491994 | TATTTGTGACGTAGGACACATGGTCTACTTCTTCTCAATATCACATCTCG |
| 1200 | 7 | 29186153 | CGAGGACCCTGCAGAACTCGAGCCTTTAAGCGCTCCTCTAGGCTCACATG |
| 1201 | 8 | 58055876 | AACAGGGAGGCGAAGACAACTTTGTTTTTTCCCGGGAGAGCCCTCGCTCG |
| 1202 | 6 | 13616537 | ATGAATTAGGCTGGAAACTGCCCCCATACCTCTAAGCTATGGGAAGACCG |
| 1203 | 1 | 116380316 | AGTGGGTTAAAACCACAACCCTTGGGGAGCCCCAGAAGTGGCTCATCTCG |
| 1204 | 3 | 62304470 | ATATGCAGGTCTCACTTTAAAAGGTCTTGAAATGGAGTTCCTACCAGACG |
| 1205 | 12 | 123713553 | CGAGAAAAGGAAATTTTAAAAGGCACAGGAAATGGTCCTACAGTAATCTG |
| 1206 | 14 | 103740065 | CGAGGCCCCAAGATCCATGTCGCCCGGAATCCCGGTGCCTAGGCCTTGTC |
| 1207 | 3 | 170136278 | CACAATCCTGGGTCGTCGGGGGACTGTACCGAGCGCCCTTGGAGTGCTCG |
| 1208 | 17 | 56355299 | TGTCAAAGGGCAGCAGGGCCCGGCCGTTGTCTTGGAAGCGCTGGTTGACG |
| 1209 | X | 151561584 | CGGATATGGTTGGTGATTAAAAATCATTATTTTCTTTAGTCTGGGAGAAG |
| 1210 | 10 | 124902844 | GAACAACTAGGCGGGATGTACTTTTGAGCCCTGCCGGGTGTCTCCGATCG |
| 1211 | X | 105066793 | CGCGCACCCAATTCAGTCGCCCGCTCCCGTTCGGCTCCTCGAAGCCATGG |
| 1212 | 11 | 124311399 | AAGGGTAGAGTGGACTTCCCAGAGCTTTAGTACCTGAGACTGGGGACTCG |
| 1213 | 12 | 8025593 | CGGAGGGCTCCCGATGAACCGAGTCACGTTTTGTAACCACATTAACGGAC |
| 1214 | 20 | 47934802 | CGCCCCCAACTCAAGGCAGGCTACACAAGATCTGTCTCCGTTGGCGTCCC |
| 1215 | 8 | 70984559 | AGGAACATCTACCCGGCTAAGAGACGCCGCCAGAGTCCCTAAAGCTGGCG |
| 1216 | 2 | 80531599 | GTCGTTTCCAATGACGGACATTAACCAGACTGTCAGATCCTGGGGAGTCG |
| 1217 | 2 | 178484058 | CGGGGTAGCATAAGAACCTGCTCTAATTCCAAGGGAATTCAAGGAGTTAA |
| 1218 | 7 | 19185165 | CCAAGCTGATAACAGGATTAGTCGCACCGATAAGGAATGCTCTGACAACG |
| 1219 | 5 | 59189791 | AGAGGTTGGTCCTTCTTCCTCCCTTTCTTCTTCCCTTCCTTTGGGCGCCG |
| 1220 | 2 | 224824500 | ACAAGGGACATCTTTTTCACAGACTTGCCTTACACAGTTTCTTGAAGACG |
| 1221 | 11 | 6976223 | CGGAAATGAGGACAGGTGAAAGGAAGAGACCCAATCTTAATTAGAACGGT |
| 1222 | 2 | 242480504 | TTGGCGTCGCGAGCGAGCGGTGGACTTGCCGGCTGGGTGCAAGCAGGGCG |
| 1223 | 11 | 18067816 | GGAAGGGCCGCCTCACTCACCTCGGGCGCCAGTAGGTGCAGGCTGGGTCG |
| 1224 | 17 | 33701353 | CGGCTGAGGCAGGCTCCGCAGACCCCGAGCCAGAGTGGGATTTAACGGCG |
| 1225 | 13 | 58207013 | CGACAAGGAGATCTGCATGATCAAGGTAGAGATCCAGGACATCAACGACA |
| 1226 | 16 | 62069806 | CGGATATCGCGAAAGACACAGCTTAGCCCAGAGCCTATGCTAAGAACCCT |
| 1227 | 13 | 96294131 | CGGGTAGTAGACATGCTTCTGGAAGGGCTGCGGGGGGCACAAAGAGAGCG |
| 1228 | 21 | 40719088 | CGGAAACTTGAAGGCCTAAAAAATTAGCTGACCTTGTTAAAAATGTTGGC |
| 1229 | 6 | 168502341 | GACCCCTGGGACAGGCAACTCTGGGGCCGTGTGACCTCCAGGACGGTTCG |
| 1230 | 12 | 110044531 | TGATTCCTGCTCCCTAAGCCTGGCTCCCTGTGGTGGGGGCTGGAATGCCG |
| 1231 | 8 | 72754469 | CGGTTAGAACGCGCTCTACATCCGCTTTAAAGACAGAGGTCTAGACGTGA |
| 1232 | 1 | 41284582 | CGGCCCCGCCCCCTGATCCGGTCCGGACTCCTAGGCCCGCCCCTACCTAA |
| 1233 | 20 | 2634874 | CGCAAGTCATCACTGCAAAGCCTGGCCCAGCCATGGGAACAACTGCATTC |
| 1234 | 5 | 1016279 | CTTCCCCAGCGTGGGGCGGAGCTCGTGGTGCTGGCCTGGAGGGACACACG |
| 1235 | 1 | 44318844 | CGGTATTGGGAGAAAGTGAGGCACCTACAGGCTAAACCTGTGGCCAAACA |
| 1236 | 19 | 46999224 | ACGCAGACCCCAGCTCAAGGCTGCACCGTTGCCATGGAGACGGTTGGGCG |
| 1237 | 1 | 88928839 | CGAGCAGCCACTCTTGGAGAAATGGGGAATGGCGCTGTAAAAGCGCAGGC |
| 1238 | 12 | 115129801 | TCATCTCTGAACCAGATACTAAAAATCCTCCAGGCGAGTGGAGAGGACCG |
| 1239 | 4 | 37246827 | CGCCAACCCTGAAGGTACGTCCCTCGCTCGGGTTTGCCCGTCCGTCCTTC |
| 1240 | 3 | 38035999 | TCAGGGATGTGGACGATGGCCATCCCAAGACTGCCCCAGCCAAGGCACCG |
| 1241 | 13 | 95354741 | GCTGCCTCCACTCTAGGATACAGATTCAGATTTTGGGAACCTGGGGTGCG |
| 1242 | 21 | 19617873 | AAGCGCTTGCTTACGTAGCTGCGGAGCGAGCCTCTGCTTCATTACGAGCG |
| 1243 | 7 | 95026073 | CGCCTCACGTCTGGCCTTGCAGAAGTCCGGAGTTCCGGCTCTGAACTGGA |
| 1244 | 5 | 1931376 | TCCGGCAGGGAGCGGACCGTGGTGGAGGCCTCTGAGCCGAGCAGGCCACG |
| 1245 | 6 | 85824216 | CGCGTATCTGAGAGCAACGTCTTTATCATCAGCACGGTGCGTTCCCTGCA |
| 1246 | X | 134125022 | CGCCTTGCAGCGCAGCTCGGATCAGCAGCAGCCCAGGAGGCCTCCCGCCC |
| 1247 | 6 | 26273535 | TTACTTGGAGCTGGTGTACTTGGTGACCGCCTTGGTGCCCTCCGACACCG |
| 1248 | 7 | 63463421 | TGAACCCCTCAAGTTTATAAATCACTTGGTAATCTTGGCCCTGCTTTTCG |
| 1249 | 6 | 100911687 | AGAGCAAACGTCAGGGATTATTATGTCGATTTTCCGAGGACCAGGGTTCG |
| 1250 | 3 | 3171664 | CACTTAGCTAAAGCCATTTCCTATGCTCCTTCCAGACAGGTTTTGCAACG |
| 1251 | 5 | 32713464 | CCATGGGTCTTTGGGTGTTGGAATAAAACCCAAACTGGACAAAGAGCTCG |
| 1252 | 6 | 27513092 | TGAATGTCATCGAAGAAGCTCCTGATTTAGATTTTAACTGTACGTAAACG |
| 1253 | 1 | 2125122 | CGGCCCTGCGCAGCTGCCCCATGTGCCAGAAGGAGTTCGCCCCCAGGTGA |
| 1254 | 13 | 113537807 | TCAAATGGTGAAATCTGAGCTTCCAAAAATACCTACTGTCGAGCACCTCG |
| 1255 | 13 | 20735532 | CGCCCCGCAAGCGGGTCGGGCCCGCCCCAACAGCCCTCGGCTCTCCTCTC |
| 1256 | 7 | 137028604 | ACAAGGTACCATCTCATGGCCTCTTGGTTCAAATCCGTTTTCTTGCTCCG |
| 1257 | 2 | 26624667 | CGCCTCTCCCGCCCGTGGGATGTCAGGTCTGTTGTCAGGCCCACGAAGGC |
| 1258 | 11 | 77734490 | ATTAATCTATATCAAAAGGTCAGTCCTTAAGGGGTCTGCTCCATTTCTCG |
| 1259 | 6 | 6007670 | GTAGGCAAGCGCAGGGGGCGGGCGGCCGAGGAGCGCTTTCGAATTAATCG |
| 1260 | 6 | 26758166 | CTGGAGAATGCAGACGTGGATCCTGCAGGCCGCTGCCTCCCAAGTGAGCG |
| 1261 | 1 | 112058304 | CGGCGCCAGGGCCCCCTCAGCGCTGCTCCTGGGCAGTCTCTGTGTCCTCC |
| 1262 | 5 | 45098028 | GCAGGGCTTCTATACCAGCAAGATTCATAAGCATGAGCAGATTTGATTCG |
| 1263 | 4 | 81188405 | CGCCGGGACACAGGCTCACCTTCCTGAGCCCAGGCCACTGGGACCAAGCT |
| 1264 | X | 91036888 | AGTGTGGCGACCTGGCAGCTGCAGCGGGACTCAGCTTTTGGGAACTGTCG |
| 1265 | X | 136629694 | GGATTTGGTCTAGGCTTTCTGGCTTGGCCCTGGAACTCTCAGCAAGGACG |
| 1266 | 15 | 68122139 | CGTCCGGGGTTCGGCTGCCTCGACCTGCCCGATTTTCCGCTGCCCCCAAC |
| 1267 | 6 | 78176622 | CGCAGCAAAACTTAGTTGAGTAGAAATAAGCTCCCGTATGAACACCGGGC |
| 1268 | 19 | 2251837 | CTGATGAGCAGCTTGCCCGCGTAGGCGGTGGGCACGCAGCAGGGTGGGCG |
| 1269 | 3 | 46925181 | AGTTCGAACTACTGGGGGAACTCAAGACTGCGTGCCTTAGACCTATCCCG |
| 1270 | 17 | 38821429 | CGCGCAGACCGAGCAGACTGCCGCAGCGACTCATCCTATCGATAAGGTTC |
| 1271 | 3 | 27772805 | CGCTGCGTCTTCTGCGGCGCAAATCTTTCGACCCTCTGCGCTCTGCCGGG |
| 1272 | 8 | 55243181 | CGCTGAGTAATAAGTGTGAAGTTTAGAACACAAACCTTGCAAGAGCTTTC |
| 1273 | 1 | 1566860 | AGCCGGGAAGGGGCGGGCCCTGGCTGCGGGAAGCCCCGTGGAGGGTAGCG |
| 1274 | 6 | 110301075 | CGCCACGCGCACCAGATCGCGCTGCAGCAGCACTGCCTGGCGCCACCCCA |
| 1275 | 13 | 36872048 | CGCCCCACCCCCTGCCGCCTGCGGGCATCGCTTTATGACGTCACCACGTG |
| 1276 | 1 | 110230252 | CGGGGCTCGGCCTCGCCAGCTTCCCTACCCAGCCCTGAGGCTCCACGAGT |
| 1277 | 12 | 40618356 | CGGAAGGTCTGGGAACTGGGAGCAGGAAAAATGAAAGCCGCCTATCTCCA |
| 1278 | 3 | 147139563 | GTGTCAACAGGGCCAAGAGGATCCAGAAGAGTGGGCCTGCACTCTTTTCG |
| 1279 | 5 | 158475854 | CGGCCCTCACCCTGTTTTTGGTGTGGAATTCATCAGCCAGAGTGAAAGAG |
| 1280 | 1 | 151694320 | CGGCCAGAGAAATGGGCCGAAATTACTGCCAGACACATCCGACCTTAGTA |
| 1281 | 6 | 150183516 | CATTCGTGCTATTATAAATCAAACAGCAACAGGGCATGATTAAACACACG |
| 1282 | 5 | 87967845 | AATTGATCAGAAGTTAGACCGGAGGCTCTGTCAAGTCACGGCCACGGCCG |
| 1283 | 6 | 32223114 | TTCTTCCCTGTGACTCAGCCCTGGAGGAATTTCCCTTTTGAAATCTTGCG |
| 1284 | 6 | 26271827 | CGCAGACTAACCGAGAAGAGAGGATTCAAAAATGAAGTGATTCATCCAAT |
| 1285 | 11 | 30039113 | ATCCCTCAGTCTCCGTCTCCTTTGCACACAGGAGTTGGGAGGCTTCAGCG |
| 1286 | 3 | 87038972 | CGCCCTTGGACATTACTCTCTGGATACAACGGAGAGTGTGCCAAGGCTTA |
| 1287 | 12 | 104852151 | CTTTGCCTGAGCCACAAGGGACCGGAGGAATGGGGTTCATTCCCACGCCG |
| 1288 | 10 | 114713108 | AATACAAAGTAAGCGAACTGTGCTTCCCCTACAGCTGCAAGCCCAGAGCG |
| 1289 | 1 | 32410614 | CATCTTTCACGTGGGGCAGCGGAGGCCTGGCGGGGACGGGGAGCCGCTCG |
| 1290 | 1 | 13910138 | CGGGCCCTGGCAAAATGGGCTTTTATTCTGCTCAACAACAGGAGGGAAAG |
| 1291 | 2 | 157178294 | CGCGCGATGCCTAGAAGTGCTCTGGTGTCACTCCCCTGTATAGGACCTGG |
| 1292 | 2 | 185462408 | CGCCCTAGAAGCGACCTGAATGGAAAAATCTGCACACGAACTAATGGGTT |
| 1293 | 10 | 91295346 | GCGGGCGGGGCATGGGGGTGGTCTGCGCGCTCTGGGCGGGCTGCAGAGCG |
| 1294 | 3 | 14852756 | GGCCCCGTGGGTCAGGTGTTGCCCCTGAGAGATGAGCAAGGACCGGTCCG |
| 1295 | 14 | 23834860 | CGCTCTCGGGGGCCTTTGGCTCCAGGCAACTTGGGGCAAGCGTCTCAGTT |
| 1296 | 2 | 157178165 | CGCCTCTCTCTGTGGAAGACGCGCTTGTCTCAAGCCCCGATGCTCTCAAA |
| 1297 | 1 | 47998999 | CGGTCTCAGCTACAACGGCGTGCATGCCAATTACGCCTGCAGGCTACCGC |
| 1298 | 2 | 3750952 | CCCAAGCAGGTGAGGCGCGGCACAGGGCGGAGACAGGTGAGGGGGCAACG |
| 1299 | X | 70150922 | CGGCCGAACTCGGCAGCTCCAACCCAACTCGGCTTAACTCCGCCTCACCG |
| 1300 | 11 | 117069780 | CGGCTGCTCCTACTTCACTGTTCTCTGGCCCCGAGTTTGAGAAGAGGGGG |
| 1301 | 7 | 137028198 | AGCCCTCCGAGAAATCGTACGTTCCTCTCACTTACTTTGAGTTGGAAACG |
| 1302 | 14 | 77591661 | CGGCTGCGGACCACAAAGACCACAGAATCCCGAAGCCGATTCCTCAGACC |
| 1303 | 10 | 13091949 | CGGGAAGAAACACAGAATCAAAGTGTTGCTCAAGGGTGCTTTCTGATCCA |
| 1304 | 7 | 31375861 | CGCGGGTGCTGAACGCGCCCTTCTAAGCTGGTGTGTTAGCACCTGTTCAT |
| 1305 | 19 | 44324747 | GCATCCCAGTGATTCTTGGGTTCCGCGTGTAGTTTCGGAAGGAGACATCG |
| 1306 | 6 | 21666666 | TCATCAGAGTTCCCCCTTTTGTTCCAAGTCCCGGAGCAGACAAAGAGGCG |
| 1307 | 12 | 22093960 | CGGGCCTCGTCCCTTAATTCTAGAAATAGCGATCGCGAAAGCTAAGTGCC |
| 1308 | 6 | 30923306 | TAATTATCCCTAATGAGGGCCCAGCCCCGGAGCTTGTTAAAGGATTTCCG |
| 1309 | 2 | 182322749 | CGCCCCAAGATAAGTTAGTTTCCCTGGCAGATTTCCAGCGAGCTGGCTCT |
| 1310 | 11 | 86383255 | CGCCGGCTGTCACCCGCACCGGATCTGCTTCCAGGGCTTGGTTTCGCTTT |
| 1311 | 7 | 19152073 | CGAGGTTGGGCCTTTGCCTGAATCTGTTGCTATTGCTCCCCTTGCTACCG |
| 1312 | 3 | 79815639 | ATCTGCGTGGGACACAGTGACTCCTAGAAGGGTATCCCAGGATTTTAGCG |
| 1313 | 6 | 43276570 | CGCCGCTGCGGCTCAGTTCCGCCCCCTCCGCAGGCTCCACCCACGGCTCA |
| 1314 | 10 | 91295338 | CGCTCCAGCGCTCTGCAGCCCGCCCAGAGCGCGCAGACCACCCCCATGCC |
| 1315 | 2 | 239974654 | CGGGAAGCCTTTCTGCCGCCCAGGCCCACAGGTCTCGAGACGCACATGCA |
| 1316 | 11 | 20692092 | GATGACGCTTGATTGTCTGAAGTCCCCACTTCGTTCTGCCTCCGGAGACG |
| 1317 | 11 | 68084453 | AATGCGCTCTCCAAATGGGGAGTCTGTTGGGGAGGCCCTGGCGGGAGCCG |
| 1318 | 19 | 11353996 | CGACGCCTGCAGCTTCTCTGGCTTCCGTCCAGCCACGCTAACTGTCACAA |
| 1319 | 17 | 38821390 | ACAGTTCCATTCTTTTCCCATCCTCCACCTTGGCTAACTGAACCTTATCG |
| 1320 | 11 | 86383713 | GGGGATTTGCGTTTGGGGGTCAGCATATCCTCGCACTCTCCCAACATTCG |
| 1321 | 14 | 92525005 | TTGCTGTTATTACACACACAGGCCTTCCTGTCTGGTCGTTAGAAAAGCCG |
| 1322 | 10 | 50816963 | AGGGGAGCCTTCCCATTTTTCTGGCCTCAAAAGTAAAGCAGCATTAGACG |
| 1323 | 10 | 1769480 | CGGGGAGCCCCGCAGAGCAAAACCAAGGAAGATGTCTTCTCTGCTCGTCT |
| 1324 | 7 | 19146441 | CGGCAAAGCTACAGAGGGAAACTAAGTCACTCGCCTGCAAAACGAGGTTT |
| 1325 | 6 | 78173200 | CGCAGGTTTGTCCCCAGTTGATAGTTCCGTGAGTTCCTCAATTATTCCTC |
| 1326 | 12 | 115136258 | CGCGCCCCACTCCGCCCAAGGGGAGAGCCCCACGGACCATTGTGGCTTGC |
| 1298 | 2 | 3750952 | CCCAAGCAGGTGAGGCGCGGCACAGGGCGGAGACAGGTGAGGGGGCAACG |
| 1299 | X | 70150922 | CGGCCGAACTCGGCAGCTCCAACCCAACTCGGCTTAACTCCGCCTCACCG |
| 1300 | 11 | 117069780 | CGGCTGCTCCTACTTCACTGTTCTCTGGCCCCGAGTTTGAGAAGAGGGGG |
| 1301 | 7 | 137028198 | AGCCCTCCGAGAAATCGTACGTTCCTCTCACTTACTTTGAGTTGGAAACG |
| 1302 | 14 | 77591661 | CGGCTGCGGACCACAAAGACCACAGAATCCCGAAGCCGATTCCTCAGACC |
| 1303 | 10 | 13091949 | CGGGAAGAAACACAGAATCAAAGTGTTGCTCAAGGGTGCTTTCTGATCCA |
| 1304 | 7 | 31375861 | CGCGGGTGCTGAACGCGCCCTTCTAAGCTGGTGTGTTAGCACCTGTTCAT |
| 1305 | 19 | 44324747 | GCATCCCAGTGATTCTTGGGTTCCGCGTGTAGTTTCGGAAGGAGACATCG |
| 1306 | 6 | 21666666 | TCATCAGAGTTCCCCCTTTTGTTCCAAGTCCCGGAGCAGACAAAGAGGCG |
| 1307 | 12 | 22093960 | CGGGCCTCGTCCCTTAATTCTAGAAATAGCGATCGCGAAAGCTAAGTGCC |
| 1308 | 6 | 30923306 | TAATTATCCCTAATGAGGGCCCAGCCCCGGAGCTTGTTAAAGGATTTCCG |
| 1309 | 2 | 182322749 | CGCCCCAAGATAAGTTAGTTTCCCTGGCAGATTTCCAGCGAGCTGGCTCT |
| 1310 | 11 | 86383255 | CGCCGGCTGTCACCCGCACCGGATCTGCTTCCAGGGCTTGGTTTCGCTTT |
| 1311 | 7 | 19152073 | CGAGGTTGGGCCTTTGCCTGAATCTGTTGCTATTGCTCCCCTTGCTACCG |
| 1312 | 3 | 79815639 | ATCTGCGTGGGACACAGTGACTCCTAGAAGGGTATCCCAGGATTTTAGCG |
| 1313 | 6 | 43276570 | CGCCGCTGCGGCTCAGTTCCGCCCCCTCCGCAGGCTCCACCCACGGCTCA |
| 1314 | 10 | 91295338 | CGCTCCAGCGCTCTGCAGCCCGCCCAGAGCGCGCAGACCACCCCCATGCC |
| 1315 | 2 | 239974654 | CGGGAAGCCTTTCTGCCGCCCAGGCCCACAGGTCTCGAGACGCACATGCA |
| 1316 | 11 | 20692092 | GATGACGCTTGATTGTCTGAAGTCCCCACTTCGTTCTGCCTCCGGAGACG |
| 1317 | 11 | 68084453 | AATGCGCTCTCCAAATGGGGAGTCTGTTGGGGAGGCCCTGGCGGGAGCCG |
| 1318 | 19 | 11353996 | CGACGCCTGCAGCTTCTCTGGCTTCCGTCCAGCCACGCTAACTGTCACAA |
| 1319 | 17 | 38821390 | ACAGTTCCATTCTTTTCCCATCCTCCACCTTGGCTAACTGAACCTTATCG |
| 1320 | 11 | 86383713 | GGGGATTTGCGTTTGGGGGTCAGCATATCCTCGCACTCTCCCAACATTCG |
| 1321 | 14 | 92525005 | TTGCTGTTATTACACACACAGGCCTTCCTGTCTGGTCGTTAGAAAAGCCG |
| 1322 | 10 | 50816963 | AGGGGAGCCTTCCCATTTTTCTGGCCTCAAAAGTAAAGCAGCATTAGACG |
| 1323 | 10 | 1769480 | CGGGGAGCCCCGCAGAGCAAAACCAAGGAAGATGTCTTCTCTGCTCGTCT |
| 1324 | 7 | 19146441 | CGGCAAAGCTACAGAGGGAAACTAAGTCACTCGCCTGCAAAACGAGGTTT |
| 1325 | 6 | 78173200 | CGCAGGTTTGTCCCCAGTTGATAGTTCCGTGAGTTCCTCAATTATTCCTC |
| 1326 | 12 | 115136258 | CGCGCCCCACTCCGCCCAAGGGGAGAGCCCCACGGACCATTGTGGCTTGC |
| 1298 | 2 | 3750952 | CCCAAGCAGGTGAGGCGCGGCACAGGGCGGAGACAGGTGAGGGGGCAACG |
| 1299 | X | 70150922 | CGGCCGAACTCGGCAGCTCCAACCCAACTCGGCTTAACTCCGCCTCACCG |
| 1300 | 11 | 117069780 | CGGCTGCTCCTACTTCACTGTTCTCTGGCCCCGAGTTTGAGAAGAGGGGG |
| 1301 | 7 | 137028198 | AGCCCTCCGAGAAATCGTACGTTCCTCTCACTTACTTTGAGTTGGAAACG |
| 1302 | 14 | 77591661 | CGGCTGCGGACCACAAAGACCACAGAATCCCGAAGCCGATTCCTCAGACC |
| 1303 | 10 | 13091949 | CGGGAAGAAACACAGAATCAAAGTGTTGCTCAAGGGTGCTTTCTGATCCA |
| 1304 | 7 | 31375861 | CGCGGGTGCTGAACGCGCCCTTCTAAGCTGGTGTGTTAGCACCTGTTCAT |
| 1305 | 19 | 44324747 | GCATCCCAGTGATTCTTGGGTTCCGCGTGTAGTTTCGGAAGGAGACATCG |
| 1306 | 6 | 21666666 | TCATCAGAGTTCCCCCTTTTGTTCCAAGTCCCGGAGCAGACAAAGAGGCG |
| 1307 | 12 | 22093960 | CGGGCCTCGTCCCTTAATTCTAGAAATAGCGATCGCGAAAGCTAAGTGCC |
| 1308 | 6 | 30923306 | TAATTATCCCTAATGAGGGCCCAGCCCCGGAGCTTGTTAAAGGATTTCCG |
| 1309 | 2 | 182322749 | CGCCCCAAGATAAGTTAGTTTCCCTGGCAGATTTCCAGCGAGCTGGCTCT |
| 1310 | 11 | 86383255 | CGCCGGCTGTCACCCGCACCGGATCTGCTTCCAGGGCTTGGTTTCGCTTT |
| 1311 | 7 | 19152073 | CGAGGTTGGGCCTTTGCCTGAATCTGTTGCTATTGCTCCCCTTGCTACCG |
| 1312 | 3 | 79815639 | ATCTGCGTGGGACACAGTGACTCCTAGAAGGGTATCCCAGGATTTTAGCG |
| 1313 | 6 | 43276570 | CGCCGCTGCGGCTCAGTTCCGCCCCCTCCGCAGGCTCCACCCACGGCTCA |
| 1314 | 10 | 91295338 | CGCTCCAGCGCTCTGCAGCCCGCCCAGAGCGCGCAGACCACCCCCATGCC |
| 1315 | 2 | 239974654 | CGGGAAGCCTTTCTGCCGCCCAGGCCCACAGGTCTCGAGACGCACATGCA |
| 1316 | 11 | 20692092 | GATGACGCTTGATTGTCTGAAGTCCCCACTTCGTTCTGCCTCCGGAGACG |
| 1317 | 11 | 68084453 | AATGCGCTCTCCAAATGGGGAGTCTGTTGGGGAGGCCCTGGCGGGAGCCG |
| 1318 | 19 | 11353996 | CGACGCCTGCAGCTTCTCTGGCTTCCGTCCAGCCACGCTAACTGTCACAA |
| 1319 | 17 | 38821390 | ACAGTTCCATTCTTTTCCCATCCTCCACCTTGGCTAACTGAACCTTATCG |
| 1320 | 11 | 86383713 | GGGGATTTGCGTTTGGGGGTCAGCATATCCTCGCACTCTCCCAACATTCG |
| 1321 | 14 | 92525005 | TTGCTGTTATTACACACACAGGCCTTCCTGTCTGGTCGTTAGAAAAGCCG |
| 1322 | 10 | 50816963 | AGGGGAGCCTTCCCATTTTTCTGGCCTCAAAAGTAAAGCAGCATTAGACG |
| 1323 | 10 | 1769480 | CGGGGAGCCCCGCAGAGCAAAACCAAGGAAGATGTCTTCTCTGCTCGTCT |
| 1324 | 7 | 19146441 | CGGCAAAGCTACAGAGGGAAACTAAGTCACTCGCCTGCAAAACGAGGTTT |
| 1325 | 6 | 78173200 | CGCAGGTTTGTCCCCAGTTGATAGTTCCGTGAGTTCCTCAATTATTCCTC |
| 1326 | 12 | 115136258 | CGCGCCCCACTCCGCCCAAGGGGAGAGCCCCACGGACCATTGTGGCTTGC |
| 1298 | 2 | 3750952 | CCCAAGCAGGTGAGGCGCGGCACAGGGCGGAGACAGGTGAGGGGGCAACG |
| 1299 | X | 70150922 | CGGCCGAACTCGGCAGCTCCAACCCAACTCGGCTTAACTCCGCCTCACCG |
| 1300 | 11 | 117069780 | CGGCTGCTCCTACTTCACTGTTCTCTGGCCCCGAGTTTGAGAAGAGGGGG |
| 1301 | 7 | 137028198 | AGCCCTCCGAGAAATCGTACGTTCCTCTCACTTACTTTGAGTTGGAAACG |
| 1302 | 14 | 77591661 | CGGCTGCGGACCACAAAGACCACAGAATCCCGAAGCCGATTCCTCAGACC |
| 1303 | 10 | 13091949 | CGGGAAGAAACACAGAATCAAAGTGTTGCTCAAGGGTGCTTTCTGATCCA |
| 1304 | 7 | 31375861 | CGCGGGTGCTGAACGCGCCCTTCTAAGCTGGTGTGTTAGCACCTGTTCAT |
| 1305 | 19 | 44324747 | GCATCCCAGTGATTCTTGGGTTCCGCGTGTAGTTTCGGAAGGAGACATCG |
| 1306 | 6 | 21666666 | TCATCAGAGTTCCCCCTTTTGTTCCAAGTCCCGGAGCAGACAAAGAGGCG |
| 1307 | 12 | 22093960 | CGGGCCTCGTCCCTTAATTCTAGAAATAGCGATCGCGAAAGCTAAGTGCC |
| 1308 | 6 | 30923306 | TAATTATCCCTAATGAGGGCCCAGCCCCGGAGCTTGTTAAAGGATTTCCG |
| 1309 | 2 | 182322749 | CGCCCCAAGATAAGTTAGTTTCCCTGGCAGATTTCCAGCGAGCTGGCTCT |
| 1310 | 11 | 86383255 | CGCCGGCTGTCACCCGCACCGGATCTGCTTCCAGGGCTTGGTTTCGCTTT |
| 1311 | 7 | 19152073 | CGAGGTTGGGCCTTTGCCTGAATCTGTTGCTATTGCTCCCCTTGCTACCG |
| 1312 | 3 | 79815639 | ATCTGCGTGGGACACAGTGACTCCTAGAAGGGTATCCCAGGATTTTAGCG |
| 1313 | 6 | 43276570 | CGCCGCTGCGGCTCAGTTCCGCCCCCTCCGCAGGCTCCACCCACGGCTCA |
| 1314 | 10 | 91295338 | CGCTCCAGCGCTCTGCAGCCCGCCCAGAGCGCGCAGACCACCCCCATGCC |
| 1315 | 2 | 239974654 | CGGGAAGCCTTTCTGCCGCCCAGGCCCACAGGTCTCGAGACGCACATGCA |
| 1316 | 11 | 20692092 | GATGACGCTTGATTGTCTGAAGTCCCCACTTCGTTCTGCCTCCGGAGACG |
| 1317 | 11 | 68084453 | AATGCGCTCTCCAAATGGGGAGTCTGTTGGGGAGGCCCTGGCGGGAGCCG |
| 1318 | 19 | 11353996 | CGACGCCTGCAGCTTCTCTGGCTTCCGTCCAGCCACGCTAACTGTCACAA |
| 1319 | 17 | 38821390 | ACAGTTCCATTCTTTTCCCATCCTCCACCTTGGCTAACTGAACCTTATCG |
| 1320 | | | |
| TSS1 | | | |
| 500 | | | |
| 1321 | 14 | 92525005 | TTGCTGTTATTACACACACAGGCCTTCCTGTCTGGTCGTTAGAAAAGCCG |
| 1322 | 10 | 50816963 | AGGGGAGCCTTCCCATTTTTCTGGCCTCAAAAGTAAAGCAGCATTAGACG |
| 1323 | 10 | 1769480 | CGGGGAGCCCCGCAGAGCAAAACCAAGGAAGATGTCTTCTCTGCTCGTCT |
| 1324 | 7 | 19146441 | CGGCAAAGCTACAGAGGGAAACTAAGTCACTCGCCTGCAAAACGAGGTTT |
| 1325 | 6 | 78173200 | CGCAGGTTTGTCCCCAGTTGATAGTTCCGTGAGTTCCTCAATTATTCCTC |
| 1326 | 12 | 115136258 | CGCGCCCCACTCCGCCCAAGGGGAGAGCCCCACGGACCATTGTGGCTTGC |
| 1298 | 2 | 3750952 | CCCAAGCAGGTGAGGCGCGGCACAGGGCGGAGACAGGTGAGGGGGCAACG |
| 1299 | X | 70150922 | CGGCCGAACTCGGCAGCTCCAACCCAACTCGGCTTAACTCCGCCTCACCG |
| 1300 | 11 | 117069780 | CGGCTGCTCCTACTTCACTGTTCTCTGGCCCCGAGTTTGAGAAGAGGGGG |
| 1301 | 7 | 137028198 | AGCCCTCCGAGAAATCGTACGTTCCTCTCACTTACTTTGAGTTGGAAACG |
| 1302 | 14 | 77591661 | CGGCTGCGGACCACAAAGACCACAGAATCCCGAAGCCGATTCCTCAGACC |
| 1303 | 10 | 13091949 | CGGGAAGAAACACAGAATCAAAGTGTTGCTCAAGGGTGCTTTCTGATCCA |
| 1304 | 7 | 31375861 | CGCGGGTGCTGAACGCGCCCTTCTAAGCTGGTGTGTTAGCACCTGTTCAT |
| 1305 | 19 | 44324747 | GCATCCCAGTGATTCTTGGGTTCCGCGTGTAGTTTCGGAAGGAGACATCG |
| 1306 | 6 | 21666666 | TCATCAGAGTTCCCCCTTTTGTTCCAAGTCCCGGAGCAGACAAAGAGGCG |
| 1307 | 12 | 22093960 | CGGGCCTCGTCCCTTAATTCTAGAAATAGCGATCGCGAAAGCTAAGTGCC |
| 1308 | 6 | 30923306 | TAATTATCCCTAATGAGGGCCCAGCCCCGGAGCTTGTTAAAGGATTTCCG |
| 1309 | 2 | 182322749 | CGCCCCAAGATAAGTTAGTTTCCCTGGCAGATTTCCAGCGAGCTGGCTCT |
| 1310 | 11 | 86383255 | CGCCGGCTGTCACCCGCACCGGATCTGCTTCCAGGGCTTGGTTTCGCTTT |
| 1311 | 7 | 19152073 | CGAGGTTGGGCCTTTGCCTGAATCTGTTGCTATTGCTCCCCTTGCTACCG |
| 1312 | 3 | 79815639 | ATCTGCGTGGGACACAGTGACTCCTAGAAGGGTATCCCAGGATTTTAGCG |
| 1313 | 6 | 43276570 | CGCCGCTGCGGCTCAGTTCCGCCCCCTCCGCAGGCTCCACCCACGGCTCA |
| 1314 | 10 | 91295338 | CGCTCCAGCGCTCTGCAGCCCGCCCAGAGCGCGCAGACCACCCCCATGCC |
| 1315 | 2 | 239974654 | CGGGAAGCCTTTCTGCCGCCCAGGCCCACAGGTCTCGAGACGCACATGCA |
| 1316 | 11 | 20692092 | GATGACGCTTGATTGTCTGAAGTCCCCACTTCGTTCTGCCTCCGGAGACG |
| 1317 | 11 | 68084453 | AATGCGCTCTCCAAATGGGGAGTCTGTTGGGGAGGCCCTGGCGGGAGCCG |
| 1318 | 19 | 11353996 | CGACGCCTGCAGCTTCTCTGGCTTCCGTCCAGCCACGCTAACTGTCACAA |
| 1319 | 17 | 38821390 | ACAGTTCCATTCTTTTCCCATCCTCCACCTTGGCTAACTGAACCTTATCG |
| 1320 | 11 | 86383713 | GGGGATTTGCGTTTGGGGGTCAGCATATCCTCGCACTCTCCCAACATTCG |
| 1321 | 14 | 92525005 | TTGCTGTTATTACACACACAGGCCTTCCTGTCTGGTCGTTAGAAAAGCCG |
| 1322 | 10 | 50816963 | AGGGGAGCCTTCCCATTTTTCTGGCCTCAAAAGTAAAGCAGCATTAGACG |
| 1323 | 10 | 1769480 | CGGGGAGCCCCGCAGAGCAAAACCAAGGAAGATGTCTTCTCTGCTCGTCT |
| 1324 | 7 | 19146441 | CGGCAAAGCTACAGAGGGAAACTAAGTCACTCGCCTGCAAAACGAGGTTT |
| 1325 | 6 | 78173200 | CGCAGGTTTGTCCCCAGTTGATAGTTCCGTGAGTTCCTCAATTATTCCTC |
| 1326 | 12 | 115136258 | CGCGCCCCACTCCGCCCAAGGGGAGAGCCCCACGGACCATTGTGGCTTGC |
| 1327 | 12 | 108238461 | CGGGCCTCAAGTCCCTGCCCGCCATGCCCCAGGGCCCTCAGCTCCACCAC |
| 1328 | X | 40441216 | AATAGCGTTACTTGTGCAACACAACATATATAATGGAAAAGGCCGATGCG |
| 1329 | 1 | 35384669 | GTCGTTGCCATGACACCGCGGGCAGGTGGGCGGCTCTGGCGGCTGTTTCG |
| 1330 | 1 | 6188120 | CGCGCACCACGTACTGCGCCACCTTGAAGGAGCTCAGGTACTCGTTCATG |
| 1331 | 11 | 30037833 | CGCCTCCAGACCTCGGTTCCCGCTCCAGCACGTTAAGCAGGGCGCACTCA |
| 1332 | 8 | 31498256 | CAAGAAGGAGGTCAGCCGGGTGCTGTGCAAGCGGTGCGGTAAGTTCCTCG |
| 1333 | 14 | 92979709 | GCCCTGGCTCTGGTCTGAGGTTGCCCTTGAACGATTTCGAGGCTCCCTCG |
| 1334 | 11 | 15136505 | CTTACTTAGCGTGCGAGACAGGCTGGTAGTGCGAGCGGCCAGTGGTGGCG |
| 1335 | 3 | 54154746 | CGCGGCCTGACCCCCGAGGCAAGGGGTCCCAGGAGTTGACCCTCAAATTG |
| 1336 | 14 | 24837892 | CGGCCCAGCCGGGAGGCGGCGCACAGCCAACAGCACCCCCACCCCCACCC |
| 1337 | 12 | 52300911 | CGGCCGCCAGACGTTGCCTACAGTCTCGGCTCTGTCTCCCACGGCTGTGG |
| 1338 | 5 | 41071405 | GAGCATGCTCAGTTCATATGACTGATTTTCACTAAGTGTAACAATCAACG |
| 1339 | 10 | 130123952 | CTCAGACCCAGCAGGAAGGGCGAGCGCACTGAGGCTGGGAAACGCAGGCG |
| 1340 | 15 | 89952272 | CGCCATGAGTCATGCAAGGACTGGGCGACAGAAGCTCTGAGCTTCTATTT |
| 1341 | 1 | 3329292 | CGGCGCGGATGCCCCAGCAGCCCCCGCTCCACTACGCCAAGCCCTCGCCC |
| 1342 | 5 | 1800606 | CGGGCGGGCCGCTGGAGTTTATCAGCTCCAGGACAAATCTGCCTCTGACC |
| 1343 | X | 101906886 | TACCGAGAGACGGCTGTGGGGGAGGCTCCTGCGGGAGAAATGGCCAAACG |
| 1344 | 2 | 39471028 | CGGTGACGAAGCTTCGAGCAGCGCCGAACAGCCCCAGCCCCAGCTCCCGC |
| 1345 | 4 | 118975504 | CGAAGCCTTCTAGATAAATACTGTGTAGAATATGGTGTGGGTGTCATTGG |
| 1346 | X | 136656581 | CGTGCGCGCTAAAAACCCACTTCAAAGTCTGTTCCCGGCACGGGGGCGGC |
| 1347 | 4 | 81185228 | AGTGGTAGGCAGGATGTAGACCAGGGCGGGCGCAGCCCTGGCGCATAGCG |
| 1348 | 4 | 81188471 | CCTTCCTGAGCCCAGGCCACTGGGACCAAGCTGATACTCAGAAACAGCCG |
| 1349 | 6 | 21666834 | TCTGCTGTCAGGCCGGGAAAAAGTGTCCGAACGCCTCGTGGACTGCAGCG |
| 1350 | 13 | 47468154 | CGCTCTGTCTGTTCGGAACAGATGTCGGCCCTCGGAGGGGTTTCTCTGCC |
| 1351 | 15 | 67326082 | CGCTCCGCGGCGGAGGCGACCCAGTTTCCCAGCTCTCGTCCTCGCCACTT |
| 1352 | 5 | 140602851 | CGAAGCTCACCGTGCCAGCCTATGTACTGTTTTCGTCTTATCCTGTAGCG |
| 1353 | 6 | 26598502 | ACCTGGCGCTCAAAGGCGAGGTGCTCGCTGAGGTGGGACCAGGTGAAACG |
| 1354 | 1 | 41158986 | CGTAGTTACTTGTATTGCTTTACATTTGCTCGTCCCAGTGTGTGAGGGCC |
| 1355 | 1 | 197887955 | CTGTGGTACCAAAGGATCTACACAGCTATGTTTGGCCGAAATAATTTCCG |
| 1356 | 15 | 67134587 | AATTTGGTGCACAAGAGGTGCACAGCAAACGCCTGTGGAATGAGTGAACG |
| 1357 | 6 | 29644756 | CGAACCTGTACAGAAGACGCTCCCATGGGTGGGCGAGGTAGCTGCCACCC |
| 1358 | 5 | 175200071 | CGCGAGGAGCAAATGGAACAGCACAAGCTGCCCGCAGCCCGCACCATCGC |
| 1359 | 3 | 136751656 | ATATCAAAGCTGGGAGAGACTCCGGCTCTGCATATGAGCGACTCTGTTCG |
| 1360 | 17 | 76101391 | GCACATGACCAGAGGCGGCCGGTAATCAGGGCAGAGTCTGGTGTGGACCG |
| 1361 | 12 | 104513155 | CGGCTGGGTTAATTCATGATGTTTGCCCTTTTAATAAAATATAAGGGTAG |
| 1362 | 11 | 2293173 | CGCGTTTCCCGGACAGTGAGGCAGGACCTAAACTCTTCCGTTAACACTAC |
| 1363 | 10 | 5734571 | CGGAGCTTGTAGCGGGTAGGGACTCCACACACCAGGGAACCCTACAGAAC |
| 1364 | 12 | 115136298 | CGGGCTCAGGACGCCTAGGCGTCCAAGCTGTGTTCCCCACCGCGCCCCAC |
| 1365 | 5 | 112073613 | CAGCCCGCCAGGGTGTCACTGGAGACAGAATGGAGGTGCTGCCGGACTCG |
| 1366 | 14 | 91580507 | GTGCTTCCCTCAAAAGGGCATAAAGGCCCCCTTTCTTCTGCTCTCCTGCG |
| 1367 | 7 | 141943158 | TCATCCCCCCGGGCGCTGAGTCCTTCATGTCCTATGGGCTGTGTAGGACG |
| 1368 | 15 | 32325334 | CATAGCATTGCCTGGCTGGGAAGTTCAATGTGTTCACAGCCAATACCACG |
| 1369 | 3 | 156008770 | CGTGAATACTCTGTGACTGGCTTGCTGAAACACAGCTTGAAATCCCTGTT |
| 1370 | 12 | 53208762 | CGGGGTCTGGTGGTGTGCACTCTGCTGCTCCATTTGCTGTCCAGTTGTTT |
| 1371 | 19 | 46387992 | ACAGAGAGCCGCAGGGGCCGGCTCTGGGTACTGCTGGGGCAGGGCCTCCG |
| 1372 | 7 | 73247613 | CGGGCATACGCTGTTTTCTGTATATGGGTTTACCCTCCAACCCATATACA |
| 1373 | 13 | 28549922 | CTGTCCTTCCCTGAGCAGCCGGGCCTAGAGTCAAGGCGCGATGATTGCCG |
| 1374 | X | 50556917 | CGGCTCACAGTGTTCCAGACCCCCCTTAAGGGTGAAGCCCCAGGGTGCCC |
| 1375 | 12 | 115136060 | GTCTAGAATCAATACGGCCCAGACAGCCCTTCACAAAGGGGCTCCAGGCG |
| 1376 | 5 | 149535695 | CGCCAGCCGCCAGCTGCTGAGTCACTTTTGTCAAAGAGTGGCCTCGGCCC |
| 1377 | 1 | 185925146 | GTGAAGCATCTCATAAACCACTGCCTTTCTGATATTGGTCTCTTTGTTCG |
| 1378 | 3 | 170137321 | GACTCAGTTCCTATTAGGTGTTCCGGTTTGTTCATTCTCAAAGGGACCCG |
| 1379 | 13 | 113568090 | CGTTGAGAGGACGTCAACGAATCTATTTGTTGGGCTGAATCCTTCCCCAG |
| 1380 | 6 | 137814960 | GTTTCTGGCGAGCAGGAGTGTGGCGATCTTGGAGAGCTTGCGCACCGACG |
| 1381 | 1 | 247694271 | CTCGGCGCGGTCTTGGTCTGGGTCGGTGGGGTTGGGCCGGGAGACCCGCG |
| 1382 | 19 | 44324951 | CGCCTACGCACTCGGAGCACAGGTTACAACTTCCAGAAACCCTTGGGACG |
| 1383 | 17 | 36021943 | ACAATTTCAAGTGTTGACAGCATCTGCTAAGTCTTCAGAATCTCAGCTCG |
| 1384 | 20 | 42955658 | GAGGCAGAGCTGCCTCCACACCCGGTGCCAGGGGGTTGGGGTTGGCAGCG |
| 1385 | 10 | 74020976 | TCCGGGAACTGGTGCACGCAAGAGCTTAACTGATTTCGCCGCGGTCTGCG |
| 1386 | X | 119134684 | GCATGAGACTCTCTGGTTGTGTTCCGTTCGCACTCAAAGTCCTCGAATCG |
| 1387 | 12 | 32138937 | CCAGCAGCCCAAGACAGCATGAGAGGTGCTAGCCCAAATATTGACCTACG |
| 1388 | 6 | 35479910 | CGCAGCCCACACCCTGGGGGTCCACCCGGGCTCTGCACCCCGCCCACCTC |
| 1389 | 4 | 101111261 | GACGGATTTGCAAAAGCAGGCGAGTCGTGCAGAGGCTCCTAAAATGCCCG |
| 1390 | 5 | 140346236 | GGGAGGGCCTACGGAGGGAGCGGCCCCAGGCGCTTTCTAGAGCGTGAGCG |
| 1391 | 14 | 36977887 | GAGATTTCAAGTGTCCACACTTCTAGTTTAGACATTTGGCAACCAATTCG |
| 1392 | 3 | 147098568 | CGCGTTCAAAGATTTTGTTTTCACGGCTGAGGGGGCTTTTTTCCAATGTA |
| 1393 | 6 | 100909443 | CGCAGAAAGGGAGCCCACTGAGACCGCAGGGGCTGACAAATCACTGCTAA |
| 1394 | 8 | 54935605 | GACCTGAGCCACTGCACCCGGCTAAGCGTTCCTTTTTTTAAAAAAAATCG |
| 1395 | 4 | 24583375 | CGATGTTAGGGGCGTTTTTCATTTTGATGTTGGTTTTTGATATTACCACC |
| 1396 | 1 | 92946825 | CGCAGGCCACTATAGGAGGCAGAACAGTAAACAGATCATTGAAAAGACCT |
| 1397 | 3 | 138679347 | TGTTCCAGGCCTGACGGGTGTTCAGGGTGTAGGACTCAGGGTGTAGGACG |
| 1398 | 6 | 32120773 | GCCTCTGTACTGCTCTGTAGTCTCTGCTAGGACATGGACGAAAAGGGACG |
| 1399 | 1 | 161007634 | CTAGCTGGGAGGTGATGGGGACTGCCTGTCATTCCTGTCAGTCTCTCACG |
| 1400 | 14 | 26674046 | ATCATTCTCCGGACTTTAAAGGACAAACTAAATGGAGACCCACCAATGCG |
| 1401 | 13 | 28549698 | TCCACAAAGCTGCCCCCACTTCGTAAGGGCCTGTGCCTGGAGGGTCGTCG |
| 1402 | 1 | 67600707 | CGGTTGCCGAGCTCTGCCTACCTCTGAAAGGCCTAGAGAGCAGAGTTCTC |
| 1403 | X | 113818537 | CGCTCTTGGGCCAAAAGCGGGATGAGGGGAGGAGCCAAGAGCAGCCAATC |
| 1404 | 7 | 44083518 | CTTGACTGAACTAGCTTCTTATCTGTATGATAGAGAAACATGCCAAAGCG |
| 1405 | 2 | 201450527 | TCCCAGGACTTACAATGTTTGCTAAGACGCAAGAGGGTGTGACACAGACG |
| 1406 | 5 | 172665707 | CGCCAATTATTGCAGCGCCGCAGCCCCTTTGTTGATACAGTAGTCCGAAA |
| 1407 | 11 | 124254360 | GTTTCACCTCCACTGCCCTGGAGGTAGAACTGGTGGTGAAGGTATCTACG |
| 1408 | 5 | 3188312 | CGGCCGCAGTGAGCCAGCTGCAGCCTGGGAGCACAGTGTGAACCTGCCAG |
| 1409 | 2 | 200819113 | CGGCGCCCAGCCCATCCGACTTTTGTAACACTCAGAATTGTAGTTTTGTT |
| 1410 | 14 | 29254680 | CGGGGCAGTTCAGCAACCCCGACCCCACCCGCGTGGCTCCAGGCCCAGGG |
| 1411 | 1 | 113286610 | CAGAGGGGTGCGGTGGGGCCTAGGAGGCTCGTGGGCGATTCCGAGCACCG |
| 1412 | 21 | 22369485 | GAGATTAACGTCACCTCTCCTGCAAGAGCGGTTAAGAGGCAGAATCACCG |
| 1413 | 6 | 43276658 | CCTTTGTCTCCCAAGTGGAGTGGTTCTAAATTTCTGATAACCAATGAGCG |
| 1414 | 2 | 154729059 | CGCGGTCCTCCCGCAACACCTGCAGCCCCGCAAGCGAGCGCACTCGGAGC |
| 1415 | 2 | 185462397 | TTTCCGTGACAAACCCATTAGTTCGTGTGCAGATTTTTCCATTCAGGTCG |
| 1416 | 4 | 16085180 | CTGAGCGCACTCCTTCCACTGTACTGGGGGTGTACAGTGAGGAGTGGACG |
| 1417 | 17 | 4690632 | GTTCTTAGTCTCCTGTCCCCAAACTTCTTAGAGTTATTCCTGTTGGACCG |
| 1418 | 8 | 146278678 | CGCAGAAGAGCAGGCTATTGGAGCAATCCGAACCCTGCGCAGCAAAAGAA |
| 1419 | 2 | 177024294 | CGGGAATCCCGCACCACGGGGAACTCTCCACCCTGTCCCCGAAGTTTTGG |
| 1420 | 15 | 37388828 | GCAGTGGCCGGGGTCTTCTCTGGCCAGATCTGAGGGACCTCTGAGCTGCG |
| 1421 | 1 | 91194708 | CGACCAAGACTTTTGCCCTTTTGAAATGAAATAGAAATAGGGGAGCTGCA |
| 1422 | 7 | 100318321 | TCTGCGGCTCTGGCCGGGGGTCGGGGCTGTTATCTGCATGTGTGCGTGCG |
| 1423 | 4 | 299022 | CGGAGTCTGCGAACGGAGTCTCCGCTGCCGCCGCTCAGCCCTCAGTCCTT |
| 1424 | 8 | 97171827 | CGGCCCGCCCAACTGCTGCCGCCGCATCTGCAGAAACCACTCGTGTTGAG |
| 1425 | 17 | 60885491 | GGTCTGCCCTTCCTCCCCTGCCGGGGCTACAGGGGTCGAGGGGCTGGGCG |
| 1426 | X | 136629816 | AGGCTGCTCGGGGGTCAGACCTGGCGCTGGCGTTTCTTTCTGCTCTTCCG |
| 1427 | X | 43741310 | CGCACCCAAAATTGCCCCCAAAGGAGGGTTCAGTGCACGGCGCTCTGGAC |
| 1428 | 18 | 14748220 | CGGGAGAAATTCTGAGCAGAGCCGTTAGCTGCTCTAACGTTAGAGCGTTA |
| 1429 | 6 | 10887003 | CGCCTATCTGTTTGGAATTTCGCAAATAAGGAACGTTTAAATGACGTAGT |
| 1430 | 5 | 2112351 | GCTGAACAACTTAACTCCTTGTCTCATAAAATTTAAGCTGCTATTGATCG |
| 1431 | 5 | 158478629 | CGGCACCAGGGCTGGATTTATAGCAAGGAGCCCCGACTGTAAGAAGTAAT |
| 1432 | 1 | 200842890 | CGCTGCGCATCATCTTCGCCATCGAGAGCACGTTTGTGGGCTCCTGGCTG |
| 1433 | 3 | 78756422 | TCTAGGAAGCATCCCATTAGTAGCAATTAAGTCAGGCAAAAGACAGCACG |
| 1434 | 5 | 502034 | TGAGTACTCAGGTTGGCGTGGGACAGGGGAGCCGGCTCCCGGGGGCCACG |
| 1435 | 10 | 134729942 | CACTCAGGCTGGCGTGGGGAGGGACACCCTTTGCATTCGTAAGGACACCG |
| 1436 | 7 | 63504673 | CGTGGATGTCTTCATCTGCCAGAGTCAAAGAAAAGGTCAGTGATACTATG |
| 1437 | 15 | 89952052 | TTCTAAAATGTGGGCTTTCTGTGTACATGGGCGCGCATTCCCAGGACTCG |
| 1438 | 7 | 158114558 | CGGCTGTGCTTATGTGCGTGTATGAGTTAGCAGCTGTGCACGTGTTTCTG |
| 1439 | 18 | 21199515 | CGGCTCCGCGCAGCAGCATCACCCGCACCACCTCGCTGTGGCCCATCTGG |
| 1440 | 15 | 68723690 | GATTATTGAATAATTGCATAATTCCGAGCGCGGGTTGGGAGGCAATGCCG |
| 1441 | 4 | 138584567 | CGGGTGCCCACCCTAGAATTAACCCTAGGATGGATGCAATCAGGATTCAG |
| 1442 | 9 | 137355128 | CGTAGCCAGCAGAGACCTCCGAGCACCGGCGCCCTCTAAAGCCGAGGTCA |
| 1443 | 2 | 127265357 | TTCTCCCAGTGCTTTGTTGACAAATGAACAAGATTTGACACTAACTAGCG |
| 1444 | 1 | 111813852 | CTACCTCCAGAGTGTGCCTTACTCCATCCAGGGACATGGATGAAGATCCG |
| 1445 | 7 | 113727612 | TAGGGAGGGCTTCTCACTTGGCTGTTACCTGGAAGTCCACAGTGGCCCCG |
| 1446 | 9 | 130566157 | CGGGGAACTCCAGAATTCGAATACCCCAGCCTTCTGTAGTTCTGGCCCCG |
| 1447 | 1 | 240257185 | CGGTGAAATGACTTGGACATACTTGTTGGTTTTCTGTGTTCTTACAAGAT |
| 1448 | 14 | 55031733 | ACCCTAAACCCTAAAAACCTCTGTACCTCCTGATATGGGTTGAATATTCG |
| 1449 | 1 | 159284298 | TTGTACCTGCTGACTCTCTCTGGCAATGTGATTATCATGACCATTATTCG |
| 1450 | 9 | 2242468 | CGTGACAGGCCGAAGAAGTCAGGCTACAAACCGGATGACAACCGTAAGGA |
| 1451 | 14 | 60973823 | CGCGGACCCCACCGCCTCTTCTGCGCGCTAAGGGTGAGCGCCAAGCAATT |
| 1452 | 11 | 2436181 | TCACCATGGCCCAGAAGTAGGTGGCCATCTCGTGGCGGTTCTGCAGCACG |
| 1453 | 6 | 72301257 | ATTTTAGTCCAACGGCCTGAGGAAACAGATACCCCTTGATCTGGAAAACG |
| 1454 | 9 | 100614879 | CGGCTACCTCTACCACACAGTTGGGAAGCGCAGTCCTAAAGGAGACGCAG |
| 1455 | 11 | 134255144 | TTGCGGAAGGGCAGTGGCCAACTCTGCTTCCTTCTGGCTTCAGGCAATCG |
| 1456 | 17 | 26835115 | CGGCGCCTCACTCACTCCCAGAGCCAGAAACCTGTTCTCCCGGGAATGCC |
| 1457 | 3 | 173302602 | TTCCAGGATTCAAGACGCTTCCTTTTCTCTTTTTTTGCTTTAAGCATCCG |
| 1458 | 14 | 86088946 | GGATGGCCGTCAGGGAATTAAATATGAATCTTTTGTCCTGTCCCACCACG |
| 1459 | 18 | 31739207 | CGCGAGGGGGAGCAGTGCTCCCAACCCGCGACCCTCCGCCTCAGACATCA |
| 1460 | 14 | 24803903 | CGGGTGAGAAAAGCTCAGGTGGGGCCCGCCGGGCCGAAGGAGGTAACCCG |
| 1461 | 6 | 117086931 | CAAACAGAGGCAGGCTGCTTCCTGAGGAACCAGCCCCTCGGGGCTTGACG |
| 1462 | 5 | 60921891 | CGGACAAAGTGCAGCGACCACGGCCCCTCAGGTCCCGCCGCCTCCTCGAG |
| 1463 | 5 | 891886 | CGGCCACGACTCCAGTTACTATGATGACAGGTCAGACCATGAGCGAGAGA |
| 1464 | 1 | 119522386 | CTCCAGGATGTAATTAGAGCTAAGAACAGCCGCCATCCCTCAGGGTTCCG |
| 1465 | X | 136628604 | CGGGCGGGACGCTCCTCTGGCGCTGTAGTGCTCCCAACTACCCTGCAGTT |
| 1466 | 1 | 50513870 | GCGGGTGTCCGTAACAGAGGGCGGGCAGAGTCGGGGGTGCGGGGCCTGCG |
| 1467 | 9 | 96623674 | CGCGGTCTCTTGAGAGCCCTGCAGAGCCCCAGGCACTTTCCGCGGGACCA |
| 1468 | 12 | 85672285 | TTGGCCGGGCGAGCAAGGCTGCTGCTGCCAGCTGATCTGCTCGGGATCCG |
| 1469 | 18 | 31739202 | TCTGAGGCGGAGGGTCGCGGGTTGGGAGCACTGCTCCCCCTCGCGGAGCG |
| 1470 | X | 47483268 | TGGCTACTTGTCCTGGCAGGCACTTCTTGATTGGCCGGCGTGCTTGTTCG |
| 1471 | X | 47483418 | GAGGCTCTAAGGGGGCTGCGCAGGGCCCAGACATTGGGGTTATGGCAGCG |
| 1472 | 9 | 135455174 | TACTGACAACCCGCTAATAGTCTTGGGCTCCAAGAAGCCCGGCAAAACCG |
| 1473 | 21 | 28218959 | GGAGACCAGTTCGAGCACTAACGCGGGGGCGCGCGAGTGTGAGGGTTGCG |
| 1474 | 9 | 139686745 | CCACAGTTGCAGGCTCTGTGGTCAGCTACGGGGTGACGAGAGTGGAGTCG |
| 1475 | 15 | 37390284 | ATTGGGGTGCGGGGCGTGCGCGCCGTAGTGCTGTGTGGCGTGGAGCGGCG |
| 1476 | 2 | 217363253 | CGCGCCCGGCCCCACAAATGTAAGATTTATGCACTTCTCCAAGCCAAGTG |
| 1477 | 21 | 43185803 | CGGCCCCATCACTCAATCAGCCGAGCATTCGAAACCAGGTCTGCCAGGCA |
| 1478 | 8 | 126448338 | CGTGAGCCCTGAGATCCTCAACACCACTGGGACCTACTCCGGAAAGGCTG |
| 1479 | 9 | 14348844 | CGGCACGATTCTCTAAGTTCGGAAACCACGAAGCTTGCGCCTTCATGCGT |
| 1480 | 18 | 61616492 | AGTGAATGGGGAGGCGGGCGGAGTCGGGCCCAGCCAGGCACCACCTAGCG |
| 1481 | 2 | 201450743 | CGCCGAGTGGGGCGGGGCTTGCTGGAGCCTAAAGTCCAACCCATCCACCC |
| 1482 | 9 | 135464814 | AGCGGCGGGGGCGGCTCGCTGGCGCCCTGGAGTCCGTGGATGAGGATGCG |
| 1483 | 15 | 53083532 | ACTCCCGAAGCTGAATTGATTTTCAAATCTGGAGGCGTCTCTCGGGGACG |
| 1484 | 3 | 13060915 | CGCAAGTGGAAAACTATGCGCTGTCCGATCGCAACCCTCCAATCTGTCCC |
| 1485 | 2 | 175193377 | CTCCAGCTCTGTTGTAATTTTGTCTTCGCCAGATAGATTAGCTGGAACCG |
| 1486 | 16 | 49378497 | CGGGAGCAGACTCAGCTGTCCCCAAACCTGAATGCAGGTAACAAGGGCAG |
| 1487 | 4 | 154144286 | AAGGTCCGGTGGCCGGAGCGTGCGGGAAGCCTGCCCAGAGTCCATTTCCG |
| 1488 | 8 | 132054555 | CGCGGCTCATCTGTCATTAGCACCGGCACTAAGCTCCCACCGCTCAGCGA |
| 1489 | 3 | 350789 | GGCTCTGAGGCTGGACCCGGGAACTTCTGTGGCTGGGAGTGTTGCACTCG |
| 1490 | 5 | 140797172 | GAGAAAAGTCAAGTTGCAGTCCCACACAGAGCCTCTGGGCGCCGCCGTCG |
| 1491 | 1 | 50891341 | GAAATCTCACGGTGACCTGGCCTCAAGCGAGAAGGATACCTGGGCCACCG |
| 1492 | 5 | 179740914 | CGCGAGAGATGGAGCTGCCCACGGTGTTGGTGACGCCCACGGTGAGAGCG |
| 1493 | 9 | 98221619 | CTCCATTCGTAGCTTTAATGCTAAGACGTTCATTAGGCGTTGCTCTCACG |
| 1494 | 13 | 97646731 | CGCCTCGCCCACTTCCCAGTAGACACAGGGACAGCGGCTTCCACTCCCGG |
| 1495 | X | 151307730 | CGGGGCTCTTCCCAAGCCACAAGTAGACTTGGCAAACTTTTTGTCCTAAA |
| 1496 | 18 | 31739423 | CATGAGCTTGGGTCTCTCGGAGGAGCAGGAGACGCGGGCTCGAAGCCGCG |
| 1497 | 8 | 105479318 | CGGCCAGGTCATAAACCCGTCAGGCCGCCGGGCTCCGGATTTGCAGCCTG |
| 1498 | 9 | 1042869 | CGCCGAAAATTTATTGACGCGGCGAGGCCAGGCTAACAACACTGCAATCC |
| 1499 | 9 | 23820979 | TTTGGAGACTTAACACCGGCCGCGTTGTGTGCGGAGGTGGTGTAGGGCCG |
| 1500 | 14 | 61655919 | CGCCGGTGTTTGGGAATCCCAGCGGGGTCCCCAGCTTGCCGATCTCCCAG |
| 1501 | 17 | 19483721 | CGCCTGGGGACACCGCTCAAATCCTAACCCTTGGCCGCCCGTCGGCTCCT |
| 1502 | 9 | 14993584 | CCCAGGTCGCTGTTTCGCGCTCTGTAGGGGATTGGACCAGGAAAGTAGCG |
| 1503 | 2 | 64549818 | AGTCGGTGGGCCAGTGCAGTTATCCTAGCAAGAGCGGGCAGAGGATGCCG |
| 1504 | 6 | 26273556 | GCTTTTACCCAGGTAACTAGTTTACTTGGAGCTGGTGTACTTGGTGACCG |
| 1505 | 6 | 100916959 | CGAGCGGAGGACGCTGTGTGTGCATAAATTTCAGCAACGCTGAATACAGG |
| 1506 | 12 | 22094226 | CGAGTTTCTAAATTGCACAGACTGCTCTGGGCCGGGGCAGACACCGCGGC |
| 1507 | 13 | 23412409 | CGCTACCGCTACCGCTTCTGCTCTCGGTTATGGCAAAAGAGCCACCATCA |
| 1508 | 1 | 1309477 | GGCGTCGCGGATGCGCCTCAAATTCAGTGCAAAAACGTGCTGAAGATCCG |
| 1509 | 7 | 8482099 | GTGGGATGTGTGAGCCGGATGCCCTCTCCTTCCGGTGTGGCGAGGACGCG |
| 1510 | 20 | 61149009 | CCCCGGGCACAGGAGCCAGCTCGGCTCACGGTGGGGTCTGCTTGGAGGCG |
| 1511 | 12 | 104609432 | CGACCTTGAAGAAGATACACGGGCTATGACTTCGCTGTTGTCACCGAGCG |
| 1512 | 2 | 187713711 | CCAACTGGGGGCGGTAGGGGCGGTAGTTACTCGGAAATCCATGTATTCCG |
| 1513 | 9 | 139916869 | CGCAAAACTTGTCGCTGCCCAATAGCACGGCCCAAGCACTCTTGGCCGCC |
| 1514 | 15 | 89952135 | CGCTTTCATTTTCAGAGCTGCCTGTGGCCTTACGCAGGTTCAGAAAATCG |
| 1515 | 1 | 92947961 | CGCGGCTTGGTCTTCACAGGCGCAGATTGGGGGCCTGTTTCATTTTTCGT |
| 1516 | 9 | 1042605 | CCGCTGGAGAAGCGGGCGCGACAGGGCGATGAGTTAACGCGGAGGGAGCG |
| 1517 | 4 | 126058 | CGTGCCTACAAGAATTCAGACGTTAGGGAATTCAAGACCATGCCTTAGAC |
| 1518 | 6 | 99276357 | CGGCCGAGTCATAGGCCTGATCATATTAACGCCCTAGGTCAGAGGGAGAG |
| 1519 | 3 | 115377744 | CGCCTTCAGCTCCCACCCGCAGCAGCAACCGCAGCCTGCTTCCGGGGACT |
| 1520 | 19 | 39466757 | CGGTTGGTAAACCGCTTAGCTGTGAGCCCGACCCAGGGCCTGATTCGAAA |
| 1521 | 14 | 20898785 | ATGCTCCTGAGCGGGATGAGGGAATCCCAGGGCACAGAGGTATCTCTGCG |
| 1522 | 6 | 43276781 | CTGTCCAGGTGTCTTCGTTACACATGTGCCTTCCTAGTCTGGAGTCCACG |
| 1523 | 2 | 98356101 | TGTCTCCACACACAGCTGGGCTGTGGTAGGGGGTGTCTCAGGCCACACCG |
| 1524 | 9 | 136114331 | GGCTCCAACACGGAGTGCGCGTGCGCTCAAGGACCAGCTGGCTAGGAGCG |
| 1525 | 6 | 10882240 | CGCGGCCAAGGACAAGGCCCCGCCCGGGTCCATAACCATTGTCCCCAGCT |
| 1526 | 1 | 90097981 | GAATCTGCCCCTTAGCCCGTATTGCAGGGGCGGTGCAGTTGGGGATTCCG |
| 1527 | 3 | 62360417 | CGCAGGTGGAATGGTCTGCAAACAGATGAATTCAGCCCCTCCAAACCAAG |
| 1528 | 20 | 21084420 | TGCAAGATGAGTCGATCAGTGACGCGTGGGGTGGCATTGTTCGCTCGACG |
| 1529 | 6 | 26271716 | CGCGCAACAAAACCATTGGCTGAAGAGTCCAACGATGTGATTGGTGAATT |
| 1530 | 19 | 51416213 | CGCCGGCTCCGCGGTCTACACCCACCCACTCCTTGGATGATCTCACCCGG |
| 1531 | 4 | 299370 | CGCTCAGAATTTGAGTGACAGAACTTGCCACCACACAGTGCACTTAATGA |
| 1532 | 9 | 131684923 | AATAAGGCCTCGGAACCCACCAGTTCAGCCCTGGGCGGGAGTCGGCAACG |
| 1533 | 9 | 134818827 | TATGGGAGGTGGTCTCTGGCAGTACTCTCTGAGTGCTGCCTCCCGAGACG |
| 1534 | 17 | 60705839 | GGCGTTGATATTTTCTTTTCCACACTTAGTCTTCAAGGAGGCTTGTGGCG |
| 1535 | 8 | 77585318 | CGGGAGGGGGACGCAGCCTAAAGACCTCGTACTCTTGCTTCTTGTTTCTA |
| 1536 | 6 | 133562056 | AGTGGCCTGCACAGGGGAACTGGGTAGCGAGAGAGTTCCAGGCAATTCCG |
| 1537 | 9 | 139880223 | CGCACAGCGCCACAGGCATTCAGGGTGTTTATTGCGCTTGGGGCCAGGGT |
| 1538 | 4 | 81189706 | CCACCGGCGGTCTGAAGATTTGGCAGCCAGCAGTCTCCAGAGGGGGGTCG |
| 1539 | 9 | 133589654 | GTAATAATAGGTACCCCTTTCCAGAAGAGGGTTCAAGATCTTCCAGAACG |
| 1540 | 17 | 40120327 | TGGCACGGGTCATCGTGGACAAGTACCGTGATGGCACCAAGATGGTGTCG |
| 1541 | 17 | 33700747 | CGCGGGCTTAGCAGACCCGCCCATTGGCTCTTGCATCTCCTGCGTGGGAT |
| 1542 | 13 | 23412250 | CGGCTAAGTGCCCCTCTCGTATCAGGTGGCCGGACACATGAACGGGAAGG |
| 1543 | 17 | 37123669 | CGCGGCCTCTAGAATATTGTAGAATCTTCCCAAGAGCCCCCGCGGGAAGG |
| 1544 | 9 | 101570703 | CGCAGTGCCTCCGCGAAACTGACAACTCCTCCGGGCGGTGACCGGCTCCA |
| 1545 | 14 | 57283436 | GCCAGGGCCGGCTGTGGTCAGGCGGGCTATTTTCTGGCAAGTGTGGCGCG |
| 1546 | 8 | 23563925 | CGCCGCCGCCACCAGCGTTGTGAACCTCTGACCCTCGCGGCTCTGCGTCC |
| 1547 | 9 | 79627216 | CGACCCGCTAGCCTAGGCCGAGACACAGTCTGTATTCTGAAAGCCGCCAG |
| 1548 | 2 | 242031694 | CGCGCCGTTGTCTAAAAAAGTCTGAACTCCACCTTAGTGTTAACTAGTCC |
| 1549 | 2 | 177022285 | GTCACGGCCGCTGACAAATAGTTCCCTTTGCTTCCTGATTTGGAACTGCG |
| 1550 | 19 | 1083429 | CGGATGAAGCCCAAGGAACCACAGGGAGATAATTTGGTTTGGACAGGGCT |
| 1551 | 2 | 65594890 | CGTTAACACTGAGCCATGAAGCCAGGGCTTGTGTCCTCAGAGAATGCATA |
| 1552 | 12 | 52626889 | CGCCCCGCCGAAGCCCAGGCTTTCAGTCCAAGCAGGGATGGTCCGGAGTA |
| 1553 | 9 | 96674333 | TAAATAAATGTCAAACACACACAAAATCCACTCTGAGGCGGGTGCTGACG |
| 1554 | 5 | 112073373 | CCTTGCTTGCTGGGGACTGGGGCCGCGAGGGCATACCCCCGAGGGGTACG |
| 1555 | X | 25034220 | GGCCTGTGGGCGCGGCGCAAAGCCCGGACTGGATTCCAGAGGGGCAGGCG |
| 1556 | 10 | 26226964 | AGAAACCTACAAAAAATTAAATTTGCCCTGTAAAACACTAGTCTGAATCG |
| 1557 | 6 | 27258466 | AATCTTGGAAAACAGAAACACCCCGAAAAATTGCAAGTTGTTTCCGCTCG |
| 1558 | 12 | 22094963 | AAACAATGCTTATCTCAAGCTGACTCAAAACTGGCTCTTGAGAAAATCCG |
| 1559 | 3 | 149374763 | CGCGCACCTCCGCCAGCAGTCCTACGACGTGACCGACGAGCTGCCACTGC |
| 1560 | 18 | 61616390 | CGGGAGTTTTCAGTCAAGAAGCGATAATGCGCTTTGGCTATTTGGGGTTT |
| 1561 | 15 | 35046650 | ATGCACAGCGATGGGGGAATGGACCATCTTGGAGAGGCTGCTAGAAGCCG |
| 1562 | 6 | 100904055 | TGTCTGGGTTAATGTTTTATAAGGGACTGGCTACTCTCGTCTACCTGCCG |
| 1563 | 17 | 32484259 | GCAGCGCGCGGCGCGGAGAGGCGAGGACCGGTATCTGGAGACTGGGGACG |
| 1564 | 7 | 32110523 | AGGGGCGCGAAGGCAGGCGCGGCCAGGGAGTGAGCGGGGCGCTGGGCGCG |
| 1565 | 6 | 100915395 | GATCGCATTCTGGGTGCCTGAGGGCCAGAGGCTAGGTTTTAAGGCCACCG |
| 1566 | 3 | 87138203 | CCGGGCAGGAGATCTGGGCTGCGGGCGCCAGGCAGAGCTGGCATCTTCCG |
| 1567 | 18 | 61616369 | TAATTTGTATGGAACCACGAAAAACCCCAAATAGCCAAAGCGCATTATCG |
| 1568 | 1 | 24648696 | GGGGCTTGGCACACAGTAGGTCTAAGGCACCGAGGCAGGAGCGTCCCCCG |
| 1569 | 8 | 38964944 | CGCTGACTGTGAATAAGTGTGTCAGTTCTTAAGGTCCAGCAACACAAAGC |
| 1570 | 9 | 115819158 | GGTCAGTCCTGTAGTGCGTAACGGAACTCGGTTCCGGCTGGCTCTGATCG |
| 1571 | 16 | 6533163 | CGCCCAATGTTTTTGCAAACTCTTGCAGCTGGGGGAAGGCAGTGGATACT |
| 1572 | 3 | 193848133 | CGGTTGGGTTTATAGAGTTGTGATCACCAAAGCCTGCATTTAAAATAGAC |
| 1573 | 7 | 19577822 | AACGGCTTAAGTCAATTATAACCTTCCTTTCAGAACCATCTTGTGGGACG |
| 1574 | 6 | 108479584 | TGAGGGGACAATAGCTGGTCTTACAAAGGCACCACCTGTGATTTAGCACG |
| 1575 | 20 | 18489660 | ATAACCGTCTCTCTCTCAAAACGTTCTTTATTAAAAGGCACTGGCGGCCG |
| 1576 | 9 | 135465030 | CGCCCCAGCCCAGTGCCCCCCGAAGGGCCAAATGCCAAGTCCACTGAGGC |
| 1577 | 1 | 1449202 | GGAGCCGCCCGTTTGCCTGGAGAGCAGCAAGGACAAAGCAATGGGCTCCG |
| 1578 | 1 | 91192798 | CCATGCCATGAGAGAGAACCTGGCAGTGTCAGATTCACTCCAGCGGGTCG |
| 1579 | 2 | 177030228 | CTCAGAGAATACCTTCCTTCCGGCAGGAGACCGTTTGGCCCTGTATTCCG |
| 1580 | X | 151129081 | CGAGACTAAGAAGTGACCCAACCTAGGTGACCCTTCAGCTAGGTCACTCT |
| 1581 | 6 | 26273096 | CGGTTTCTCATTGGTTCTTTTAGTGTCTGAATATATGATAGGCCATTTCT |
| 1582 | 2 | 9898228 | GTCCCTTCCCTGGCTCAGGGCTGGCTGGAAAGACAGAGACGTTTCATGCG |
| 1583 | 5 | 343927 | CGCGGTCCTGGCGCCCCCACACCCGCGACCCCGCGAGGCCCCCACCTCCT |
| 1584 | 6 | 100895618 | AGATTCGCTTTTCTAACTACGGAAGTAAGGCTCTGGGCAGCTGAGCTCCG |
| 1585 | X | 38080303 | CGCTGGAGACAAGCCCCAGCAGAGCGAAATATCCTGAAGTAGCTTTCTTT |
| 1586 | 6 | 32096716 | CTAGGGCAGCCTCAGTTGGCAGCCCAGAGCTGTGACCGGGTGTTGGAGCG |
| 1587 | 2 | 187714068 | CGCCCTTGACTGAGGAGTGTTTTTAATAATCATCTGGCCGACTTGGAAGC |
| 1588 | 6 | 106429316 | CGCTCCCACCGCTACGCGGGGCCGCCTGGCTCCTGGTTCTCATCGTTCTT |
| 1589 | 6 | 26251899 | TGTATTCTTACTCCTTTATCTTGTTGCAATGCCTGATCCAGCTAAGTCCG |
| 1590 | 2 | 163695111 | CGCGCGAGCCGCTCTTTGTGGCAGAGCATCCTCTTTTGAAACCAGAATTC |
| 1591 | 6 | 26250826 | AACTGTTTGTTCAGTTGTGACCATTGCTTGAAACCCATTCCTATGGCACG |
| 1592 | 4 | 188916724 | GGGTTTGCGTGCGGGCCGGGTGCCTGGGGGCCCCGGGCTGAGGGTGAGCG |
| 1593 | 17 | 7492801 | TTGGAGATCTCGGAGTTGTGCATCTTGGGGTTCTGCTGCGCCATCTGGCG |
| 1594 | 18 | 13611370 | CGCTGCAAATAACCCCCACCACAAGCACTGCCGGGAGCCCAGAATAGCCT |
| 1595 | 17 | 40120496 | CGGCCATTTCAAAGAGCTGCTCCAGCCGTTCCCGTTCGTGGTTGGTGTCA |
| 1596 | 1 | 81897627 | TCACCACACAATTGGGCTTTTTACCTCTCTTTAAACAGAATGAAATGCCG |
| 1597 | 18 | 13611481 | CGGATGCCCCCAGGCTGTGTGAAGAAAATTCAGGGCAGGCTGCGAGTCTG |
| 1598 | 12 | 57037657 | TTAATAACATACCAATTTTGCCACCCTTGGCATAGGGAGCTAGCAGATCG |
| 1599 | 7 | 44185323 | CGGGCGACCGCAAGCAGATCTACAACATCCTGAGCACGCTGGGGCTGCGA |
| 1600 | 11 | 86383761 | CGGTGCTCAGTGTTGCCTTGTGCTGTCCAATATAATAGCCATTCGCCACG |
| 1601 | 12 | 127211262 | CGGGCTCCAGGACCGTCCCCTGTGCGCCCTCATCTGCACCGTCGCCGTCC |
| 1602 | 18 | 60985636 | GAAGTCGTCGCCGGCCTGGCGGAGGGTCAGGTGGACCACAGGTGGCACCG |
| 1603 | 2 | 21023042 | TCTTGGAGGGTGTCGGGGTTTTCCTTTCTTTTTCATTTGAATTTTATCCG |
| 1604 | 6 | 32223098 | AGCCCTGGAGGAATTTCCCTTTTGAAATCTTGCGGTGTTGAGTAGGACCG |
| 1605 | 14 | 62583919 | CGGCGTTCCAGCTCTCAACCTTGCTCCCAGCAGCCCCCGCCGGCGCACTC |
| 1606 | 6 | 108436768 | TCTAATGAGAGATGACGGCCAGGTGTTCTCAGCAGCCCCTGGCCCTCACG |
| 1607 | 14 | 95240444 | CGGAGATGTCCTCCAGAAAGCAGCAGAGCCATGGAACGGGGTTTGAGCAC |
| 1608 | X | 105067038 | TGGAGTTTGGTGCGGTCCCGGTGCCCAGGGGGCGCCTTTTCCGCATGCCG |
| 1609 | 4 | 9917839 | GCATTACAGGGTTCCTGGTGTGTTCCTCTAACCACTTAGTTGGGAAATCG |
| 1610 | X | 63614857 | CGCGAATGGCGGTTACAAGTGCAGCTAGTATAAAAAGCTGAGATAAGGGT |
| 1611 | 2 | 202122669 | TAAATGCTTTCCAATAAAGCATGTCCAGCGCTCGGGCTTTAGTTTGCACG |
| 1612 | 8 | 99986964 | CGGGTCACTCAACTAAGAGTAGCGTCCAAAGGCAGGATGCGGTAACACCT |
| 1613 | 7 | 105319819 | TCAGAGGAGTATCAGAGCCAGCGCTGTGTGTGCAGGGCTGGGCCTTTCCG |
| 1614 | 12 | 85673270 | CGGCCTCGTAAAGCTCCCAGCTGGCCCGGACCCCGAGCTCCTCCCGGTTA |
| 1615 | 10 | 22040307 | AAATCTAAGTAAGCTGGTCCACTGATGAAAAACCTCCTTCCTTGCATGCG |
| 1616 | 6 | 3232246 | CGCCGCGGAACTCACAGAGGTTCCCCCGCCATAGCCTCCCGCGTGCGCTC |
| 1617 | 10 | 73976790 | TCCAGTCCCGAGCATTTCGGATGAATGAATGATGAACAAACGACAGAACG |
| 1618 | 17 | 41363789 | CGCAGCAGCCTGAGACGCTGCCAGCTCTTCAAACGTTACCCGAGGGCCCG |
| 1619 | 7 | 1121929 | CTGTCGGGTGCAGGTGGACGTCCCCTCACGGTAAGGTCAGAGTGCTCCCG |
| 1620 | X | 63005077 | GCTTGCGAAGTCCGGCTTCTCTGAGGCCCCGTAGCTGGCGCGAGTTGTCG |
| 1621 | 3 | 149374761 | GGGCAGTGGCAGCTCGTCGGTCACGTCGTAGGACTGCTGGCGGAGGTGCG |
| 1622 | 2 | 223176606 | CGCCCTGCGGGGAGTCGCCTTCGTTTAGTCCCAGGTTTCTCCGGCGCTTT |
| 1623 | 17 | 38821487 | GGAGCCTCGCAGAGTTTTAAAGATGCAGAGCTCCTTTTTCGGAGGCCACG |
| 1624 | 3 | 62305003 | AAGTGCTGATAGTGGGCCTCACAGAAAAGTTACCATGAACCGACTTTCCG |
| 1625 | 3 | 13974467 | GAGAGAACGGAGGTGCGGATGCGGCGCAAGCAGAGGCGCAGGCGAAGGCG |
| 1626 | 2 | 102803290 | AAGTCCTTCATCACCAGGAGTGTCTGCTTAAGTCTATTCCAAGTTGAACG |
| 1627 | 6 | 26252265 | CGAGGGCACTAAGGCCGTCACCAAGTACACCAGCTCCAAATAAATGGACG |
| 1628 | 18 | 53446784 | CGTGAAGTGGGTGCTTCAGCTGTCCCCCAGCTTTAGGATTTTAACCCAGC |
| 1629 | 5 | 1800542 | CGCGGAGTGGACGCAGCCACGTCTGCAACTCACCCGGACACACACAAAAA |
| 1630 | 11 | 69451412 | ATGCGCTCCGGAACCGTAATGCGCGCTTTTTCTAAGCCTTACGGTAAACG |
| 1631 | 5 | 528580 | GCACATGCGGATGCGGAGAGAGGCCCATGGCGAGTTCGGCGGCGTGGCCG |
| 1632 | 1 | 2163529 | CGGGGCCTCCCAGGCCACTCCTTCTAGGACAAGTAGGGTCTGGGCACTGT |
| 1633 | 18 | 56932872 | GCGAACCAGGTTGGGCGGGGCCTGGGCGGGGCTCTCAGATAGCGAAGGCG |
| 1634 | 11 | 77184194 | CTGTCCAAGCCTGGAAGGGGTCATTTATTCCATGCAGAAGGAACTGGGCG |
| 1635 | 1 | 155035375 | CGCCTCAGACGAGGGCGGGCTGTGTGGGGCGGGAGTACCAGAAAGGGTCG |
| 1636 | 5 | 76923943 | CGGGACCTCCCAAGTAAGGGACAAACGCGCTTCTCCAGAAGAGGCGTGTC |
| 1637 | 2 | 37571729 | CGCGCAGGCTGCAGCCCTCCTACCCCGGACCTTCGCAAACCCCGGGGCTG |
| 1638 | 19 | 36604614 | CGCATGGAATCTGGTGTTTATTACACTCGGGGGAGAGAGGAGGTCACTCG |
| 1639 | 19 | 39466295 | GCAGCGGCGGGCTAAGCTTCCCTTTCCCACTTTGTTCTGGGAGGCAGTCG |
| 1640 | 11 | 1029188 | AGCTGGGGGCTCTGCGAGGGGGCGGGGCTCAGACACGGGTGGGGTCACCG |
| 1641 | 19 | 51231736 | CGCAGTAACCGGATGACTCCTACGGGTCACCTCGCAAGCCCCGCCCCCTC |
| 1642 | 6 | 35479628 | CGCGAGGCAGCGCCTCTACCCGCTACCCCCAAGCCTGGGCGCACCCCCTC |
| 1643 | 7 | 44923719 | CGAACCTCAGTTGAGAAACAACAGAAGCTGAGACAATTTTACGCAGGTGA |
| 1644 | 17 | 41797729 | CGCCAGCATCCGTCAGGAGGATGCCTTTGATAACAAAATTGACATTGCTG |
| 1645 | 12 | 115136096 | AACTGGAAGCTATGTCGCCCCTTCTTTCAGGGCCCAATTAATTCGCCGCG |
| 1646 | 10 | 8097689 | CTGCGCCGCGTCCATGTAGGAGTGGCTGAGGCCCGGGTGGTGCGTGTCCG |
| 1647 | 7 | 8482107 | CGCCACACCGGAAGGAGAGGGCATCCGGCTCACACATCCCACCCTATGTC |
| 1648 | 17 | 35298199 | GGCCTGGGTGCCCAAAGTGGCAGTGTGGGCCTCTGTGGGATGGAGAGGCG |
| 1649 | 13 | 109149011 | ACTGCTTTTCTGTAAATGACAGAAATACTAAGAGAACTTGACGGAAATCG |
| 1650 | 6 | 162259067 | GTCTGCATGTGTCTCCTTAGCCTTAACCCCCTTGCCTAGGACAGAAAGCG |
| 1651 | 11 | 62620843 | CTGAACCACCAGTGATGAATTGCTGCTCACCAGTGATGAGTTGAATACCG |
| 1652 | 6 | 78172337 | GCACGTCGGGAACCCGCGAGTTAATAGAGGTGACCGAGGACGTGGACCCG |
| 1653 | 7 | 29186231 | GCTGACTTGATGCGGCCAGGTTTCTGGGACCAGGAGGTTGGGACACGACG |
| 1654 | 7 | 27282987 | CGGACACCGAGTCGGATTTCTATGAAGAAATCGAGGTGAGCTGCACCCCG |
| 1655 | 1 | 1289835 | AGCAGCAGCGTGGCCAGCACGTAGCCCAGCTGCTGGAAGAAGTGGGCTCG |
| 1656 | 6 | 26250685 | AGCTTGCGAATCAGTAGCTCAGTCGATTTCTGATAGCGGCGGATTTCACG |
| 1657 | 6 | 104216442 | CGGGCCTCTAAAACATGAGCTCTGCCTTCTGGGATCAGTGCACATAGACC |
| 1658 | 12 | 66275988 | GCGGGCTGGCCTTTCTGCTGCTGGTAGGAGGATCATGTGCTGCTATTTCG |
| 1659 | 6 | 32016535 | CGCTTGTTTTAGAATCTGTGCCCTGCATTGCTGTAAGCAGCTCACAAACA |
| 1660 | 4 | 107995 | CGGGCAGAGGAACAGGAGGAGAAACTGCCGACAATCCCTCATTTCCAATG |
| 1661 | 17 | 75316383 | CGCCGCTGCTAAATATATCCGTAGGAATGGAGAGGGACCGGATCTCAGGT |
| 1662 | 6 | 50683213 | GGGTGCATTCCATACTGAGAGCCATGGCTCATGAGGGACAGGTCATGGCG |
| 1663 | 5 | 172751061 | CGGATTATTGCCTCTAAAAGGCTGGCCTGGCACCCTGCCTGGCAGCATCT |
| 1664 | 2 | 25149977 | CGTGACGTCCCGCAGAGGCCTCGGAATTACTCAGCATTCACATGCCGTTT |
| 1665 | 4 | 3390332 | CGCCCAGCCCCATGGTCCTGGAGAGTCCCCCGCTCCAGCCCTGGACCCTC |
| 1666 | 14 | 91580280 | TCTGTCACCCAGGCATCAGGATTTTTCAAAATTACCCCTCTGTTGAGCCG |
| 1667 | 17 | 37608096 | CGGCCATGACTAGATTTTCAAAGGCTATTGGTATCAGGAGTGTCTTATGA |
| 1668 | 15 | 29967531 | CGCTGCGTGGGAGTTCCCATCCCGCGGTCCCAGGACCCCCGGCAGCAGCT |
| 1669 | 13 | 111040242 | GGAGCTCTGACCTGCCCTGGCTTGGCTGTGCCTGGGTTTGGGACAGTTCG |
| 1670 | 3 | 110627980 | AATTCACCTGAGGCACTTGTTAAACCCAACTTTCCCAGGCTCCTCGTTCG |
| 1671 | 16 | 31214401 | CTGGGCTGGCTCTCACTGGGTTTATTGGAGCACCTAGGCTTAGAACCTCG |
| 1672 | 5 | 50265600 | CCATTTGCTTATTCTCACTATTGTTTTAATTTATTGTGCAGCCTGTTGCG |
| 1673 | 2 | 10220802 | TTGCTGAACCCTGCGGGGGGCCCGGCTGAGTTTGTTCATTTATGGAAACG |
| 1674 | 10 | 119312767 | TGTAACTGCGCATTTTTCTAGACCAGTCTTTATTATTCCAGCAATCAGCG |
| 1675 | 3 | 147140880 | GAAACTGCAGCGTTTCGGCCTCACAGATGGGGCTGGAGTTGGAGACGTCG |
| 1676 | X | 114957471 | GAAGGTCCTCTGCACCAGAAAGGCTGCCCATGGCACAAACCCTGATCTCG |
| 1677 | 19 | 8952029 | CGCAGACCACAGAATCCAAGAGTGCTAGGAATCAGAGGAGAGAGAGGAAG |
| 1678 | 5 | 140420518 | CGCATTTTGTCGGAATAAACCAACACACGGAGGTTGGGTAATCTAGCCCC |
| 1679 | 11 | 129871059 | AGCAAAGTTCCTTTCTAAATACCCCATATTGAATAAACACTGTTTAAACG |
| 1680 | 10 | 134944446 | ATCTCCAATGCAAACCCAGTGTGTGGGTGGCTGGAAAACACACTACTACG |
| 1681 | 18 | 32173227 | GGTTTCACATTCCATTGAAGTCCCACTGGCAGATGCCTTTGGTAAGCTCG |
| 1682 | 10 | 121300857 | AAAAAGGTTTACATGACCACTAACGCTAACGGAGCACTTAGTACTTGCCG |
| 1683 | 11 | 27015813 | AACAGAGCCAGCATCTTGCCAGGGCCCCGGAGGAGGGGTTCCCCGCTACG |
| 1684 | 1 | 116371187 | TTTTCAGCAGAACAGACGCAGCCCCCGAGTCAGTGCCGTCAGAAGACACG |
| 1685 | 4 | 112709411 | TATGTTGCTGGTGCGGCGGCTCCGCGGCGTTGCTGGCGGCGGGGGGAGCG |
| 1686 | 1 | 173639130 | CGCCACCCAGAGACTGAAATTATAGATAATTCCTGGCTGTTTTCCACTCT |
| 1687 | 6 | 168773765 | TATTGATAAGGACATTTCATACCTAACAAGGGCCAGGTGACATTTCCTCG |
| 1688 | 1 | 155831789 | TGACCAGAACTGCTTTTATTGACACCCCCTTTCCTAAACATAGCTTTTCG |
| 1689 | 1 | 203598761 | CGCCCCGCCAGCCTCTCCGCCGCTGGGCTCCACCCTCCCGGGCTCTGCTG |
| 1690 | 2 | 43865055 | TATCGACGGGTAAAAAGTGTTGTCCTTTAAAAAGTGCCCCACTAACTTCG |
| 1691 | 7 | 87229552 | CGCGCTGGAGGTGAGACTAACCTCTAGTCCCCCGTCGAAGCCAGAGAGCA |
| 1692 | 11 | 77734334 | CCTGCCACATGCAGATCACTTGGGCCAGCGGAAGTGCCCCTGGCTGCCCG |
| 1693 | 19 | 48918116 | CCCTTGCAGCAGCGCTTTTCCGGGCGGGGGGCATCCGGTGGAGGGCTGCG |
| 1694 | 17 | 37321490 | GCTGGGTGGGAAGCAGACGCGGTACGGTGGGCAGAGGTCCCCAGCCTGCG |
| 1695 | 5 | 50694985 | CGCCTTGTTTGGATTAGCCCTCCAGATCTGCCAACCTGATCAACTCCTGA |
| 1696 | 14 | 103524899 | CGCGCCCCAGAAGCTCTCAGATACCCCCACGATTTCGGTGACCTTCCGCA |
| 1697 | 10 | 26223206 | GGGCGGGAAGCCGGGCGCCTCCTTATTTCTTTACGGGGGCGCTGGCTACG |
| 1698 | X | 72668487 | CGCCCACTGGCTTGCTGGTTAGGCTGTGATCGCGATCGCTTTGACTTCTC |
| 1699 | 17 | 79919578 | TGGCCGCGGTGAGAGCGGGGCCCCAGGGCTGCGCGAGGTGGGGCGATGCG |
| 1700 | X | 43741557 | GGATCGAGGCCCAGAAAACGGAGCAGCGGGCACCAGGGAGGCCTGGAACG |
| 1701 | 14 | 104569653 | GGCTTCACCCTTGAGACTGTGTTGCCCATTGCTCGGCCATCGGTCTCTCG |
| 1702 | 19 | 51522588 | GGGAGATTCGGGCTGGAACAGCGGTAATGGGCACAATTACCCTAATGACG |
| 1703 | 12 | 63025948 | TTTCTCAGCCTGGCTTTTATTTACACTCTTATTAAGGAGGCTTTCATTCG |
| 1704 | 8 | 61835708 | GTGGCAGTGCCACCTGGGCCCGGACTCTGGGACTGCGGGAGGCGGGAGCG |
| 1705 | 6 | 21666659 | AGTTCCCCCTTTTGTTCCAAGTCCCGGAGCAGACAAAGAGGCGGCGAGCG |
| 1706 | 11 | 124190997 | CGAAGTCAGGGAATAAGAAATCTCCAGTTCAGATCTCACAGCTTTGAAAG |
| 1707 | X | 51139025 | CGCCAGGCTCCTGACCCCAGCTTGGACAGCAGGCCGCCGAGCGCCCCTGA |
| 1708 | 10 | 26506713 | CGCTCCCGCCTACCCGCAGCCAGGAAACCCCGACTCCGCTGACTGGGATG |
| 1709 | 3 | 99594931 | CTTGGTGGAGCGAAGTTGCTCTCCCGGGCTTACCTGAACTGCCTGCGCCG |
| 1710 | 6 | 31938777 | CGGTGTCTCCACTTCACTCAGGTATAGTGTTCCCTGGAACCGGGAGGCTG |
| 1711 | 13 | 102569894 | CGAGCCTTCCGCGGCTTGGTTGGGGCTTCTCGGTCTTAACGTGGTTACCG |
| 1712 | 13 | 23309774 | AAATCCTGACATAGATTTCAACACACCTTATGATTACCAGAGCACAGACG |
| 1713 | 5 | 50260129 | ATGCCATAAAACAACAAGCCGGTCCCTGGGAGCAAGTAAAAATTTATTCG |
| 1714 | 19 | 46916520 | ACACCACGTTTCCAGCTTGGAGTGGGCCTTGAGCCTTGGGACTGACCTCG |
| 1715 | 5 | 139228150 | CGGGTGCCGCAGTAACGGCTGCTGCTCGGCCAGGCGGTAACTGATGGGCG |
| 1716 | 4 | 15780306 | ATCCTGAGATGAGGTGGGTTGGCGACTAAGGCGCACCGGTGGGCACTGCG |
| 1717 | 22 | 39377790 | CCTGGGCAGTGAGCCTGTTTATAGACACCCTTCTCCCTTCCTTGGTGTCG |
| 1718 | 5 | 153859646 | CGGGGGGTTTCCAGCCCGCCTCGGGCAGGAAACCAGGACCTCCCTGCACC |
| 1719 | 12 | 59990794 | CGCCACCTGACCGGGCCTGGCGCCCCCAATGTGGTGCAGTGGCCCAGCAA |
| 1720 | 7 | 78982418 | TGCCCCCTAAACTGTTGCAAGTCATAGCCCAAACTCTGTAAGCTAAATCG |
| 1721 | 4 | 81106857 | GTCCCCTTGCGAGGGCACTAGGACAACAGGAGGGGCGCACCTGCAATACG |
| 1722 | 7 | 134143906 | CGCCCTTTCTGCCGACCTCACGGGCTATTTAAAGGTACGCGCCGCGGCCA |
| 1723 | 12 | 3862428 | CGCCCCGCCCTCGCCACCCAGCCTCCCTGGCGGCCTCAGGTCGCGCCTGC |
| 1724 | 1 | 41174793 | ACATGATGCCCTAGTGCAGCTGCTATGTGCATTTGCCAGACATTTCCACG |
| 1725 | 3 | 13060741 | TCTGTCCAGGATACAGCCTGCAGCCAGGATCGTCCCTCTCAGAGTCCTCG |
| 1726 | 17 | 76227996 | CGAGGCCAGTGCGCGGGCAGGAGCGGGGACGTGCTCAGAAGAGCCGGGCG |
| 1727 | 13 | 28544760 | TTTGACAGCGCTGCCAGATTCGGGAATGAGGATCACTGCGGCGAGGCCCG |
| 1728 | 17 | 79495953 | GCCGGCTCACGCTCAGCAGGTGGGCCTGCGGGTGGATGGCCAGGTGCACG |
| 1729 | 14 | 24803917 | CGGCCCGGCGGGCCCCACCTGAGCTTTTCTCACCCGCTCAAAGCCGCTAC |
| 1730 | 7 | 124092424 | CGGAGTGACGTGAGGTGAGATTTACAGGCTGTTCAGTACATATCACAAGT |
| 1731 | 4 | 41881829 | CGTCTTCACTCAGGGCTTTGGGGATCCCCTCGTCGCCCCAGGTCAGTGGG |
| 1732 | 5 | 140420475 | CGGCCAATACCTCGGCTTGCACAAAGCTGCCTGTCAGCTTTCCTAAGACA |
| 1733 | 5 | 145720805 | CGGGAGTTTGTAGTATTCATAACCGTTGAGGACTCTCTGACCCCAGCTCG |
| 1734 | 17 | 79448940 | CGGGCCTATCATTGGCTTAGGTTTTAGGAAGGTCCCAAGCTCATTTGCCA |
| 1735 | 13 | 28501366 | GCGAGCGCAGATTTGCCCCACCCCAGAGCCCAGGCGGGTGTGGAGAACCG |
| 1736 | 2 | 219848528 | CAATCGTCTGGCAGAGTTGGCCAGCAGAGATCAGCCGGCGTGAGTCAGCG |
| 1737 | 3 | 125899401 | CGGGTCTGCACCAACCTCAGTTTCGGGGCTGCGGCTCACAGAAGTCTAAC |
| 1738 | 5 | 444228 | CGGGGAACATCAGCACCCACGGAGACACCTGCGCCAGGGAGACACCTCTG |
| 1739 | 12 | 53359506 | CAAGCCGAAAACCAGGAGACTCTTGCAGTCAGGGGGCAACTGGGGAAGCG |
| 1740 | 20 | 49576406 | TGAGGCCTGACGTCCAGCAACAGGTGGAATGCCCCAGAATCCAGCAGTCG |
| 1741 | 1 | 152671707 | GACAGGTCCTGGTTGCAAGTTTCTCCATAAGGGTTGCTCTGTGATGTTCG |
| 1742 | 7 | 56562731 | GACCTAGATGTGAGCCACACCTTCACAAGCCATGCCATGTTCAGAGAGCG |
| 1743 | 17 | 75315837 | CGGGACAGGGAAAATGAAAGACTTTGGAAGTCGTCAGGAATTTGACTCTG |
| 1744 | 7 | 1086220 | CGCTCCAATTTGAGATCCCAAAATGAGATGCTGCCCGGGACCAGAAGTCA |
| 1745 | 7 | 29847171 | CGCCACCCTCCAAAGGCGCTGTTCCTGAGAGCCGGTGCCTGCCTGCCCAC |
| 1746 | 4 | 77935993 | TGTCAGCTTTTGCAATTATTGGAATGATGTTCACCTGTAAAACAGACACG |
| 1747 | X | 90689603 | CGGGAAGTTACTTAGCACGGTTCCGGGTTTCTCGCGCCCCGCCTGTCCCC |
| 1748 | 14 | 60386050 | CGAGACCTGCGGTTGCTTTGGTTTGCCTTTTACTAAGTTCATTTCTCAGC |
| 1749 | 12 | 85672623 | CGTCCTGACACAGCCTTTGTCAACACTGGCACTCACAGCGGCCGTCGCGT |
| 1750 | 5 | 176296727 | ACAGGGTGGCGCAGGCTGTTTTCTGGACATTCTGCCCCTCACAGAAAGCG |
| 1751 | X | 25033893 | CGGCGATCCGCTCTCTGCGCCCTGGGAAAAAGCCCCAGCCATGAGCAATC |
| 1752 | 10 | 102880841 | CGTTTCATCGGTAGTACCTTGCCCCAGTCGCTGGCTGCCCTTTGCCCTGT |
| 1753 | 7 | 45025080 | CCGTTTCAAAACTGTTCTAGCAAGCACTGAAGGAATGCATAGGGTCACCG |
| 1754 | 14 | 95155784 | CCCCTGCTTAATTTCCTGGGTGTTCGTATTTTAGGCTCTTTGCTTCCTCG |
| 1755 | 7 | 19018055 | TTTTCCTAGTAATTTATTCTTCAAGGCCCATCAATGTAGTTATAAAAACG |
| 1756 | 14 | 24641501 | AGCTCTGCATCTCCAACCTGGAACCCAACCCAGAAGTCTCAAGTTTGACG |
| 1757 | 7 | 18329562 | CGTCAGAGCCAGGGCCCCCAGTTGTGGAAGTTTCTATATCCGGTAAATTG |
| 1758 | 16 | 1428639 | TTCCCGGGCTTCAGCACAGGTGACTTGGCTGAGGTAGGTGGTGGCTGACG |
| 1759 | X | 25034151 | TGGAGTCTCTCTCCTCCACGTGCTGAGAGGCGCCCTCTGATTGGCTCTCG |
| 1760 | 4 | 174430614 | CGCTGCGGCCCCCGCTACCGACACCGTCTCGACCCCGCGCATTCTATCCG |
| 1761 | X | 119444909 | CGCACGTCGCTCCCGCTGCAGCCAGGCCGCTCCCCTGGGACTCTGGTCCA |
| 1762 | 11 | 117070046 | CGGCGGCAGCCCTTTAAACCCCTCACCCAGCCAGCGCCCCATCCTGTCTG |
| 1763 | 17 | 37123671 | CTCCTTCCCGCGGGGGCTCTTGGGAAGATTCTACAATATTCTAGAGGCCG |
| 1764 | 14 | 38066873 | CTTCTTCAAAGCCCAAGACCGGTGATGCAGAATGGACTCTGCCAGCTTCG |
| 1765 | 20 | 62321231 | CCAGGACTACGGAGCTGTCTTCCTCTGTGACCACAGGTGCGTGCAGTCCG |
| 1766 | 6 | 100896674 | CGGGCGTGGTCAGCACTGCTTTGGGGCTAAGCAGAGTTTCAACATGTTTC |
| 1767 | 11 | 829783 | CGGCACTGCCCAGGGTACACACACCTCACCGACCACGCACGCCGGGCAGG |
| 1768 | 3 | 44063404 | CCCGAGGTGGCAGTGGGCTCCTGGTACAGCGGATGCGAGTCCGCACTCCG |
| 1769 | X | 118107876 | GGCAGCGCTCGCTGGACAGTGAGGAACTCGCGGAGAAGATGCGCTTGCCG |
| 1770 | 19 | 41260294 | CGCACCCGGCCAGAACCCTGGTTCTTTAGGACAGTGACAGCCCAGAGTAG |
| 1771 | X | 151128222 | CGGGATGACTGCAAAGCATTTGTATGGATGGCCATCTCCAATCATCCCTA |
| 1772 | 6 | 43211213 | CGCGCCGCCAGCATCAGCACCAGCCCTGACATCACCGCCCGCCGCCCGCT |
| 1773 | 2 | 182543233 | CGCGGCGCTAGACAACCTGCGCAAGGTGGTGCCTTGCTATTCTAAGACGC |
| 1774 | 3 | 39188743 | CGCCCTACAGCCCGCACAGCCCGCACGGCCCTGGTATGGTGGCTCTGCTT |
| 1775 | 2 | 162284135 | CGCACTGACAGTGTCATTTCACCAGACACCCAAGACTTCTTGGAAGCGAG |
| 1776 | 10 | 111216962 | GCATCGGGTCTGTCCGCAGCCGGCAGCGGTGCAGAGCGGTGGGGAGAGCG |
| 1777 | 6 | 26757706 | CAGTTACAAGAAGCCGAGAGGTCTTCTTGTCATAAAATTAACTTCCCTCG |
| 1778 | 20 | 21000992 | CAGAGGGAAGCCGGGGGGTCTGTGCTGGGATCTGAAGGTTTGCAGCCTCG |
| 1779 | X | 119445209 | CGCCCCCGGCTCTGGGCGCCCCGCAGAGCATCCTACTCCGCGGTTGCCTC |
| 1780 | 14 | 52536436 | CGGCCGCTAGGGAAGACTTGGCCACCCTCTGGAAGTACCTTCCCCCGGGA |
| 1781 | 11 | 134731585 | TTTCCGCCCGTGTTGCAAATGCCCGTGGTTGGGTTGGGGGCGGGACAGCG |
| 1782 | 14 | 70235567 | TACCGTCTATCATTTCGAGGTCTGCGACTACTAAAACGGTCAGAGTATCG |
| 1783 | 1 | 2125186 | CGCCCGGCACCTGATCCCTGCAGGGCCCCCAGGGTGGAGCAGCAGCCGTC |
| 1784 | 3 | 77089518 | CGCGCTGGCAAGTTTGTGGAACACTTTCTAGGAATTAGGTCTTTTCCTCC |
| 1785 | 1 | 50886782 | CGCCGCCAACGGCATCATCCCCCCGAGGCCCGCCTACGAGGTCTTCGGTT |
| 1786 | 12 | 85667616 | CGGTGTCCCCGATCGCGCTGAACCACTTCCCAAAGCCTGCGCCCTCGCCC |
| 1787 | 12 | 21926524 | CGGGATGATACTCTTTCTGGCCAACATCGTCCTGTCACCATAGCCAGCTT |
| 1788 | 20 | 738228 | TGGTGCATGAAAGCAAAACCAGAGAGGCGCCTTTTCCAAATGCCAATGCG |
| 1789 | 4 | 111544463 | GCTGGGCAGGAGCTGAATTCCCGGGAGCTTGCCTGTGTAGACCCAGTGCG |
| 1790 | 19 | 17357619 | AAAGGGCTTGGCCTGGCGGCCGGCTGGACTGCAGATGTCTCTTCCAGGCG |
| 1791 | 20 | 5485284 | GCTGTGCGTTTGACAGGCAGTTGGGAGACCCTGGTGGCCGGTTTTGGTCG |
| 1792 | 3 | 147108843 | GGGCGACACGAGGGCAGACGGTGTAGCCGAATCGTAGCCAGAGCTGGGCG |
| 1793 | 20 | 62795807 | CATTAGCAGCGGTGGCACGGGCTGGCGGAGGAGGCTCCTTTGTAAATACG |
| 1794 | 8 | 80992750 | CGGCACTCAACTGAAGGCTGATATCAGGCGATTAGACAGCCATGCATTCT |
| 1795 | 5 | 83680326 | TCTCTTTAGTCACCACTCTCGCCCTCTCCAAGAATTTGTTTAACAAAGCG |
| 1796 | 14 | 85996364 | ACTAACTTAACCCTGTGTGCGCCTGACACTCCCTAGAGAAGCTGGGATCG |
| 1797 | 8 | 11561498 | AGGCTCTGGGGTCTCCGCGGAAAATGCCCAAGTGCTACCGCCAGGGAGCG |
| 1798 | 14 | 29237480 | CGGCCTACCCCAGCCACCCCATGCCCTACAGCTCCGTGTTGACTCAGAAC |
| 1799 | 1 | 201251982 | CGTGGTGTTTTACCGGGGTATACTCATGAACAACAAATAACCACCGGTTT |
| 1800 | 20 | 6750617 | TCTGGTTCAGGAACTTGGGACCCTTCATTATCCCAAACGTTTGAGCTTCG |
| 1801 | 11 | 6942045 | CGGCACTGTGGAGAGAGGACTTGGAAAATAACTAAGTTGGTTTAAATTGC |
| 1802 | 7 | 3083333 | CGGGGCGCCTCCTCCACGCAGGCCTCCGGCTTCCTCCAGGCGACCAGGCC |
| 1803 | 19 | 12991665 | CGCCTCAACCCAGCAAGGCTCAGGACTCTTCCATGCGTGGGCACACGAAG |
| 1804 | 3 | 10184319 | CGCCCGGCCCAGAGCATTCTTTATAAGGCCGAATAGTTTGCATTTGAAGG |
| 1805 | 1 | 6262620 | GCTCAGTTCCCCCTCTCTGCTCATGGTTACCTAGTGTGGTCCTCAGCTCG |
| 1806 | 2 | 66664667 | CGTCTGGGATGAATGATCTTGCCCTAACTTGTGTCCCTATCAACTAGTTT |
| 1807 | X | 151374946 | CGGGACTATGCAAGCAAGGAGGGAGAGGAGCCATTTGCAGGTTTCACACA |
| 1808 | 1 | 88271572 | CCCTGAAAATCAAATGGCAGAACCTAAACGGAAGACTCACTGCCAAAACG |
| 1809 | 11 | 3073441 | CGGCCCCGACGCCCTCCAGGGCTGAGTACCTACACTCACACACACGGCGG |
| 1810 | 1 | 110692918 | CTTAGCTTCTTGAGGCCCCTGGTCAGGGGATGCAGACCCTGGAGCCCTCG |
| 1811 | 6 | 30095326 | CGGTTGCTTTTCAAATACAGCTTTTCTAAAACGCCGCCAGGCACCCGGGC |
| 1812 | 7 | 29186183 | CGCTCCTCTAGGCTCACATGACCCAGACCCACAGGGCAAAAAGTGCGTCG |
| 1813 | 4 | 85402497 | CTTCATACTCCCAGACCCAGACAGAGAAGTTTCCCGAGTCATCCACATCG |
| 1814 | 1 | 92191625 | TTGCTCATCCCAGTGCAAGATCTGTTTATGTTCCTCTCCTTGGCTGTACG |
| 1815 | 11 | 6953558 | CCTCTGAGCAAGTTGATGGCTATCTCAAAACCTCGAAACCTGTCTCTACG |
| 1816 | 7 | 49815502 | CGTCCACGCAGCCCTTGCCACGGTAGTCCGGGTACACGTACTCCTCGGGT |
| 1817 | 3 | 159756929 | TGCTCTCACCAGGCTGCGCGCTGGCGGGTCGGTGCACAGGGACGGGGTCG |
| 1818 | 11 | 34492984 | CGGGGCCCTGCACCTGTGCAGCGAAGCTTAGCGTTCATCCGTGTAACCCG |
| 1819 | 8 | 65284438 | CATTTAGTTCCATCGAAAGGGAATGTAACAATATAACTCACCACTTCCCG |
| 1820 | 7 | 100318080 | CGGGAGCCCAGCCTTTCCCAGATAGCACGCTCCGCCAGTCCCAAGGGTGC |
| 1821 | 12 | 115136226 | CGGACCATTGTGGCTTGCACAAAACCCGCGCCCCAAGAGCCTCTGCCAGG |
| 1822 | 1 | 177133566 | CGTCCACCACGACCATTTCTCCCGGGAAGTCGTTGCGCACCGAGAAGAGG |
| 1823 | 1 | 18707479 | GGCTCTATCTCCTTGTGGTCATCATAGCCCATTTGGCCCTGGTCAAGTCG |
| 1824 | 6 | 7052860 | GTGTGCGTGGGGGGTATGCAGCATCCAGTTACATTCCTGACCCACTTTCG |
| 1825 | 6 | 121758980 | CGACAAGAAAGATACTGAATATCGCCCTCCGAAAATCGGGCGCATTTGAA |
| 1826 | 6 | 49518986 | CGGGTGGAGGATTATCAGGTAACACAAGAAGTGTTGCAGACCTCCTGGGC |
| 1827 | 7 | 29186267 | ACCCTGGGGCTGCTTGCTTCTTCCAGCATGGGAAGCGCTGACTTGATGCG |
| 1828 | 2 | 176969218 | CGGTGATTTTGAGTCACCCAAGATAAGACACTAACTTGACCTTAACTTTG |
| 1829 | 4 | 206562 | CGGCTTCAGGGATGTCGTAGAGTCTTAGCTACGAATCATCCAATACCCGC |
| 1830 | 3 | 181444989 | CGCGGATTGCCGAGGGCTTGCAGGCACTCAGGCAGACGCAGACAGAAACC |
| 1831 | 4 | 188916668 | CGGGGCCCCCTGACCCCCGCGTTCACCCCTCAGCCCGGCCGCCGGGCACT |
| 1832 | X | 2886295 | TGGAAGAAACGTCTTCTTCCTGCCAAAACAGACACGCACTGGCTGTCTCG |
| 1833 | 7 | 54956419 | TCCTTCAGTTAAATAAGAAATCAGGTCCAGAGCCGAGTTCAAGGCATTCG |
| 1834 | 3 | 29323182 | CATCCAGTTCCCTGCCTCGGAGATAAAGATTCCAGCTACATGGGCAAACG |
| 1835 | 13 | 51417469 | CGGGGGAAGGGCAGCAACTCGGCGCCCCCCTCAGCGCCTCGCACTACCTC |
| 1836 | 15 | 35086890 | GGGTCCCGAGTGGGACGGGGGGCTCGGCGGGAAGTTTACCTGGTGCCGCG |
| 1837 | 18 | 74799572 | AATCATCTGGCTTCTGCAAAGCAGTCGCGCAGCGCTGTAGGGTGAGGCCG |
| 1838 | 10 | 119304487 | TTACAGACTGTCCTGGCGGTGTCTGCAAGTCGGTAACCCCCCGGGTTTCG |
| 1839 | 3 | 147126703 | CTGCCCTTCCCGAAACCAACTTTTCGCTGAGCGATTAGAGACTTCAGACG |
| 1840 | 1 | 67218135 | GTCCGGCCGGGGAGGGCCGAGGGCTCTGTTTGAGGTCACCCTGGAGACCG |
| 1841 | 11 | 63743837 | CGGCCCATCCTGGTCATGTTACTGCATTTGTGGCCGGCCTCCCCTGGATC |
| 1842 | 6 | 100061111 | CGCGCCTTCCCAGGCAGGAACTTTGCGACCCCACCCCCTGGGCCCGCCAC |
| 1843 | 8 | 67090581 | CAGGCGCTTCGCAGGTGAGCCGGGTGCTGCCGCCTCTCTGCAGAGAGACG |
| 1844 | 12 | 103359572 | CGCAGCCAGCCCCACCGCCTCCGGACTCGACCCCACCATAGCCCCGCCGT |
| 1845 | 6 | 26250934 | CTATTTATAAAGGCAAAGTCCTTCTGATTGGTCCGAATCTTCCAGTTTCG |
| 1846 | 5 | 146461855 | CGGGTTATCTGATGGAAGTAGTATAGAGTCATCATTAAAAGTAGAGACTT |
| 1847 | 5 | 154026930 | CGTGGCTGATCACCCCAAGAGAAGTCAACCCCAGGCGGGCGGTTACGACG |
| 1848 | 7 | 101006019 | GAGGTCTGGCTTTCGGGGTGTTAGGCTCCAGGGACCTCCTGCGGAGCTCG |
| 1849 | 7 | 135615376 | AACATACAGGCACAAAAGGGCACATACACACTCTCAAAACCAGAATGCCG |
| 1850 | 6 | 100912946 | CGGGGCCGACTCGTGTCACTCAGCCCTATTGGCCAATGAGCGCCTCGCCC |
| 1851 | X | 23350552 | GCTTTTCCCTCCGCGATCATGGTGAATCAGCAAAGCGCTGCAGAGCAGCG |
| 1852 | 6 | 27259010 | CGCCACAAAGACGTCAGGGATTAGGGTCGCAGGCGCCTTGTTTAATGGCC |
| 1853 | 6 | 29760164 | TGGGGAGTAGCTAGGGGCCTGCCCGGCGGGGGCGCAGGAACCCGGTTGCG |
| 1854 | X | 38080364 | CCCCAGCAGAGCGAAATATCCTGAAGTAGCTTTCTTTGCCCCAATTTGCG |
| 1855 | 14 | 85996495 | CGTTCGAAACCTGTCGCCGTCACTTGCGCGTTTGGCATTATCCATTGTCA |
| 1856 | 6 | 33169600 | AGCCCACCGGAAGCAAAACTGAGCAAGATCTGAAGTAGAGAGCGATGCCG |
| 1857 | 11 | 124056244 | CGGCTCAGCCCCATAAGGTAGAGCAGCATTTTGGGACAAGTCACTGAGGA |
| 1858 | 3 | 147141588 | CGGCGCATGCTTGGGACTGAGTCTACAGAACTACTTCTGGAAAAAATCCT |
| 1859 | 5 | 87975812 | CGGCACTTCTGTGTTCATGTGTGTACAAGGTGGTATGTAGCCCACGCATG |
| 1860 | X | 113816304 | GAGAGAAAGTTTGCTAGCCACGCGGCTTGCTGAAGCACGTCTGGGCTTCG |
| 1861 | 7 | 15725196 | GGACAAGTGGCTGTGAAATCGATTGTGAAGCGTGACCTTCCATCCCAGCG |
| 1862 | 3 | 147125714 | CCCTCGATTAACCTGTCTTTGCCTAGCGCAAAGTCTATTCAACAACTGCG |
| 1863 | 5 | 76924190 | CGAGAGTTCCTCAGATGAACGGTGTCCCTGGGGCAGTGGCGCGGAGGGCG |
| 1864 | 11 | 57269634 | CGCTTTACTCAATGCTCAGTGAGTCATGAGGTGGCTGCCCCCAGGCCTCC |
| 1865 | 17 | 684490 | CGACGAGGAAATACTAACAAGGGACAGCTCTGTGGTTTCTGCTATCGAAA |
| 1866 | X | 119443277 | CGCCAAGTAGCGACTCTTTAAGCCCTGGGCCACCAACCAGCCCACTCCCC |
| 1867 | 4 | 3371566 | CGGGATCTGGCACACGCCTAAAGCCCTCTCAGTGCTATCTGTATTCCGTG |
| 1868 | 7 | 6099250 | CGCCCGGCGGCATCTCCCGTTTTTTTGTGGCATAACAGTTGACCAAAATA |
| 1869 | 1 | 19615358 | GCAAGTGCAGAAGGTCCTAGTAGCCGAGCGTGGAGTCGAGCTCGTGGGCG |
| 1870 | 13 | 45008526 | CGGGACCTCATGCAGCTTGGCAGATATCTGAGAAATGGTTTAATTCATGC |
| 1871 | 18 | 60985645 | GCCGGCCTGGCGGAGGGTCAGGTGGACCACAGGTGGCACCGGGCTGAGCG |
| 1872 | 2 | 219848580 | CGGGCTTCCCAATCACTAGACCCTGGCTACTGCCACCTGCTTCACCCTCC |
| 1873 | 7 | 45148557 | AGTGAGAGAGCCGGATAAGTACCATTGCTGCTTGATTGCTGTCCAAGTCG |
| 1874 | 7 | 2143942 | TTCGCGGAGGGGCGGGGCTGTGGGGGCTGTCTTCCTGTGTTTTCTCTCCG |
| 1875 | 5 | 32712623 | GTCCATCTACAGTTTCGACGAGACCAAAGACTTGGATCTGGAAGACATCG |
| 1876 | 13 | 53312966 | GGCTTTCCGCCTTAGGTCAACGTTGTAGAAAAGCAAGTGAAGGTTTCACG |
| 1877 | 9 | 111929547 | GGGAGGCTGCGGTGCTGGAGCGAGGATGCGCAGACCGAGGCAGGGAGGCG |
| 1878 | 11 | 113778957 | CGGCAAGTGACATGACATCATTCATAAAGAAATGGGTATAAACAGGATTG |
| 1879 | 8 | 25282664 | CGGTTGCACCTCTGGGGTGGAACATCTATAAAATTTAGATAATGATACCC |
| 1880 | 4 | 114214093 | CGGGATCCCAGTTCGTTTAATTGCACCGCTTAGTTTACATTTCAATGGCT |
| 1881 | 2 | 166262886 | CTCTGAAGTTTGGGCTCATTGCCAGTTTAAGTATATGCTGCCACCTTTCG |
| 1882 | 16 | 49311934 | CGCTCATTCACAGGACTTTATTCAAGAGGTTCGTCCAGGAGTTTTGATTG |
| 1883 | 6 | 163746145 | CGTACAGATCTTTTCCAGAATAAAGCATTATTCGGCCAGGACGCCTCAGC |
| 1884 | 6 | 10886967 | CGGGGCAAAAACTTGGAACAGCTACCACAAAGCGAACGCCTATCTGTTTG |
| 1885 | 14 | 37136192 | CGGCCCTTCAGAAGCGCCTTCTTTGGTTAACTCTACCCCCAACGAGAGCC |
| 1886 | 19 | 3587038 | TCGGTGAGGCGGGTGGGCTGGCGGGAGCTCTTCCCGAAGTGCGTTCTACG |
| 1887 | 19 | 44325008 | AACGAAGTATGCCATCTGTTTCCGTTATCAGTTTATTGCTCTCACCTCCG |
| 1888 | 14 | 96180847 | CGGGACTCTGGGCCTCTGCTGGGAACTCACAGTCGACACCAGGATGCCCA |
| 1889 | 6 | 27858637 | CGATGATCTGATCCTGATTGGTCAGAACTGTTGTAGGAAAAACCTGGTTG |
| 1890 | 19 | 46544711 | CGTGACTCTGAAAAATTATAGACAGTGCAGGTCTGCAGGTGAACTGAGAC |
| 1891 | 1 | 203598594 | CCTATTTTCAGTAATCTGATTAGGGGTCGATGTTGGTGGGCTCGGGGACG |
| 1892 | 7 | 121940381 | GCCCAGGTTCTACAGAGTCCAGGGATCTCCTTTCTGCACCCACCGTAGCG |
| 1893 | 17 | 1957496 | CGTCACCCACACAAACCTGACCTTCACGTGGGGCGCGGGACCTTGCGGGG |
| 1894 | 20 | 39659249 | CGAACAACCCTGTTGAAGGAGAAGTTACCTTCTGTAGTGTGCTGGGAATT |
| 1895 | 1 | 200010137 | CGGGTTCCCATAGCAGCCAGACCCACAGGTCAGCCTCCGGGGCTTCAGCT |
| 1896 | 8 | 11568257 | AACTTCTCTCTTTTCCTTAACCAAACACCAAATTCCCTCCAGGATTGTCG |
| 1897 | 13 | 110522297 | CGGGTGAGGCATCGCTCCTGAAGAATTAATCAGAAACTCAGAAAAATTCC |
| 1898 | 16 | 30908233 | CGGTGATCTAAGCCCTGAGCCCAGGTCCCTGAAACCTCAGCCCCAGGCCT |
| 1899 | 1 | 200009927 | TGTTGAGAGCAGAGTTCAGGGCTGGTGCACTCTGCGGTGCTGAGTGGGCG |
| 1900 | 1 | 235814009 | CCTGCGGTGCCACAGGGACGAGGCCTGGAGAGCAGTCGCTCCTAGAACCG |
| 1901 | 17 | 4853706 | CGGCCCCAGCCCATCAATGAATGGGGCTGAATCAACAGTGAACAAAAATG |
| 1902 | 5 | 158478734 | CGGGGTGTTCCTGCTCCGTGGCCTCCATCCCAGCCGATCCCGCTTTGCAG |
| 1903 | 2 | 23633680 | CGGGAAACCACACGTCTAAAGCGTCCCATCTGTAAACTCTTGCTTACTCT |
| 1904 | 4 | 299121 | CGGAGTCTCAGCTACGAATCATCCAATACCCGCAGGTCACAGAGCGACGG |
| 1905 | 8 | 137806993 | ACAAGATTGGATTCGCCCCTGCCACTTTAAGAACTCGTGAGTCTACCACG |
| 1906 | 4 | 13530923 | CTTTTGGGCGCGGCTCGGGGGTGGAAGCCCTGGGCGACAGCACAGCTTCG |
| 1907 | 6 | 105401673 | GAGGCGGGTTGCGATGGCAGCGCTGGGCTACAGACCCCGCCTTATTTGCG |
| 1908 | 1 | 116711077 | AAGTGGGAGCCCTTCCCCCATCCCGGGCGTTGTTAAAACCCAGTGGGGCG |
| 1909 | 1 | 1602914 | AGCTCACCTGAGAATCTGCACACCCAGATGCAGGTGAGCACCGGGTCTCG |
| 1910 | 1 | 63784454 | GTGGCTTGAATAAGTCGTTGGCGCGATGTGAGATAGAATCATCATGCCCG |
| 1911 | 1 | 79085713 | CGCAGTTGTTCTGAGGAGAAATTAGGAAAAGGTATGCAGTTACACAATGC |
| 1912 | 6 | 32223226 | CGTGGTATCTCGCAGTTTGGCTGAAACCTGAATTAAATGCAATGCTTTTT |
| 1913 | X | 25034241 | GCCCGGACTGGATTCCAGAGGGGCAGGCGGGGGTGGGGGCAGCGGGCACG |
| 1914 | 6 | 99275335 | CGGGTCCCTCTGCTAGGACCGCGCCTTCAACGCGGAATCGCTGACATGAG |
| 1915 | X | 119130048 | CGTGCTGTGAAACTGGACTTTGATTTAACCCCTGCGAGAGGAAATTCTGA |
| 1916 | 7 | 56951337 | CGTCCTGCCCAACTATGTAGCGTGCAGGCCTCCATCTTCCACATGACTGC |
| 1917 | 4 | 188916865 | CCTCCCTAACCCTGGCGGAGCTGATGGGTGGCTGTAGCCTGATTAGACCG |
| 1918 | 18 | 59001158 | CGTCGAGGTGGCAGGGAGTTGCTGGGAGAGCACAGCGCGGAGAGGGACCG |
| 1919 | 3 | 11643427 | CGCACCGGCCCTCCCCCAATCAGCCCCAGCAAGAGGAAGTTCAGCATGGA |
| 1920 | 5 | 50265443 | TGACCCGGAGGACGCGCCTACGGAAGAGACGTCTGGTCGAGGGGGTGCCG |
| 1921 | 4 | 140201433 | GATGGTGAGGACTACAAAGGTCTCCCCACCTCCCGGGGGCCGCGGAGGCG |
| 1922 | 14 | 85997519 | CGCAGGCGCTCTAGAAAGGCGTGATGGCCCCAGCTCAGTGCCCGCCAGCC |
| 1923 | 2 | 8724060 | CTTGAACATGACCCTGCGAGGCGGGCCGGGAGGGGGCGTGGCATGCTGCG |
| 1924 | 2 | 119607603 | TCAAAAGCTCTGTCACATCGCGCTGGCAAGACGCTTAATCAAAGTGAGCG |
| 1925 | 8 | 70947218 | CGGAGCTCTACATCAGAGGGCATTTAATTGGCCTTGAAATTTTCGATCGA |
| 1926 | X | 25034093 | CGCCCGCTGCTCACTACACTTGTTACCGCTTGTCCTGAGCGCGGAGAGGG |
| 1927 | 17 | 33701321 | CGGAGCCTGCCTCAGCCGCAGGCTCCATAAACCCAGCCAGGAACCAAGCG |
| 1928 | 15 | 53097649 | CGGCTGCGGATCAATACCCGCTCACTGTAATCCCAGTAATTGTAGGGCTC |
| 1929 | 19 | 51128640 | AGAACGTGGGGCTCAGCATCGCCGTGGTAGACTACGACTGGTGAGGGGCG |
| 1930 | 2 | 154729622 | GGGCTTGCTCCCCTAACCAAGGCGCTTCGGGATCGCGGGGCTGGTGTTCG |
| 1931 | 4 | 160514445 | CGGATACTTTCCAGGGAAGGAAATAAGACAGGAACATGTGCCCTAGTGGA |
| 1932 | 21 | 22369908 | AGTGTTTCCTCTACATCTCGACCCATTGCTGTTGGGAGCGGCACCTTACG |
| 1933 | 20 | 21682362 | CGCGGAGTTTCGTTGTGTTGTTACACAAGCCAGCTTTTGGTTTGTTAGTT |
| 1934 | 6 | 79620363 | CGCCCTCCGCGCCGAGAATGCGCCCTTCTGCCCGGGCTTGATATCACTTT |
| 1935 | 10 | 1769525 | CGAGCAGAGAAGACATCTTCCTTGGTTTTGCTCTGCGGGGCTCCCCGCAG |
| 1936 | 11 | 7961111 | CGAGTATGAAGTCGTAATCCCATAGCTGTGAGTTCAAGTCTGGAATTCTT |
| 1937 | 8 | 110986685 | CGCGGGACCAAAGCTTCCGGCGTGTCCCCAACTTTGTGGCGCCCTCAGGC |
| 1938 | 7 | 149389929 | AGCGGCTCCGAAGTGGACAGGTGGCTCCCCAGGGTGCGGAGGCGAGGGCG |
| 1939 | 14 | 101317620 | GTTGCATGAACGAGTGGTGTGAAGCCTGTTGGGTAGCTTGGCCACTCCCG |
| 1940 | 15 | 25454202 | TGATGGTGTTGCAGGCTGGCCCAGGGACTTGTGGGCTTTGGGCCAGGTCG |
| 1941 | 4 | 85416865 | CGAGCCCTCTCGGGCCCCGCCAAGTATTTTGTTTGTTCGAAAGTCTTCTC |
| 1942 | 14 | 38676865 | CGTTGCTTTGCTCGGTAAGATCCCTGCAAGCCTTTATTTGAAACTAGCCT |
| 1943 | 3 | 139918866 | CGCTGGGTACCTCCTACAAGGCCTTTAGTCCAGGCATGAAGGCAGTTCAG |
| 1944 | 11 | 111385659 | CGCTAGGGGGATACATCGACGCCGAGATCCAGAACTGCTTAGCCAAGTTG |
| 1945 | 6 | 10887023 | AAACTTGGAACAGCTACCACAAAGCGAACGCCTATCTGTTTGGAATTTCG |
| 1946 | 6 | 29975366 | CGCGGGTACCAGGGGCCATGGGGAGCCTGCTCGATCTCCTGTAGATCTCC |
| 1947 | 7 | 41026197 | CGTGTGCTGTAATTCTAAGGAAAACATTAGTAGCCTATTTTTAGCAAGTA |
| 1948 | 7 | 121956890 | CGCCTCTCGGCTCTCAGCAGCTCCAAGCCCCCGCCACAGCCCTCCGCCAC |
| 1949 | 6 | 28677272 | TTGTTTAGAAGAGTGAAACCTTCTGTCTTGCCGTGGGCCTGGTGAAAACG |
| 1950 | 11 | 87908785 | GGAAACTCTAAAGCGCCGGATAAATAAGCGCTGCAACAGGCAACCACACG |
| 1951 | X | 91036807 | GTATCTTTAGACCTTAGCGCATCCATCTCTCGGTTCTTTCGTCAAGACCG |
| 1952 | 1 | 2125243 | CGGCTGCTTCACGGGGCAGGAGGGCACCTGGAATCCCCCGCCAGGTCCCT |
| 1953 | 5 | 176107201 | CGGGGTTCGAGTCGCAGCCTCCAGCCTCAACCCGCACCTCCCGCCCTGCA |
| 1954 | 11 | 55322753 | CGCCATTTTGTCAAGGGAAAAAGCAGATGGAGGTCGTGTATATACAAATA |
| 1955 | 18 | 32173206 | CCCACTGGCAGATGCCTTTGGTAAGCTCGCCAATGGCAGGCTTTCATGCG |
| 1956 | 18 | 3726858 | GGGATCGCATTTGCTCCTTCTTGGACTCTATGAATCTTATCCTAAGTACG |
| 1957 | 17 | 38821155 | CGATGCAGCATCTATTGGGGAAGGAATTTAGCTTCTACTGCAGTGTGACT |
| 1958 | 19 | 51522894 | CGGCGACTCCCAGGTGAAGCTACCTGCACCCCACCCGGGTTGGGGTGGAT |
| 1959 | 5 | 496299 | CAAACGCCGCATAGGTGGGAGGGCGGCGGTTTGGAACATTCTGCCATCCG |
| 1960 | 19 | 36132561 | CCCTACTGGGCCTGGTGGAGGATTGGGTGGGCCTTCACTCCTGCTCCACG |
| 1961 | 7 | 55304314 | CGGAAAAAACTATCCTTGAGTGAGGCCTGACACTGTGCCTGTGATGATGT |
| 1962 | 6 | 26273182 | CGGTATCACTTCGGCTGCGAACATGCCTGAACCAGCTAAGTCAGCTCCCG |
| 1963 | 17 | 33842301 | CGCCGGCGGGACAGGCACTTGGAAAGCAAGATCCAGGGTTCTGAAGATTA |
| 1964 | 5 | 3188172 | GCGGTCTAAAGTGTTTACTCTGCTTCTTCTCAGAAATGGCTTTGATAGCG |
| 1965 | 14 | 57265560 | CGGGCCTGGGGGGTCACCCACAGGAAAGGTGAGATTAGCAGCACCATGTG |
| 1966 | 21 | 22369802 | AGCCTCAGCTGTGTCCTTAGAGGCCTGAAGTCTCTCCTGGCCTCACTCCG |
| 1967 | 3 | 69915406 | CCCCTAGTGACACAGCCAGTGCCAGAACTAACTTTGACTTTCACTCTTCG |
| 1968 | 15 | 96897414 | CGCACTACGAGAAGGGGAGTTTGATCTTGTCTCATTGGCGTCTCGGGTCT |
| 1969 | 7 | 25890808 | CGGAGACAGCGGCAGGGATGCGGCTCTGCTCTACAGGCAGACAGAAACTT |
| 1970 | 7 | 105319558 | TGGCCAGGGCCCTCATCCAGGAGCCAGGAAACATCTACTACCACGAGTCG |
| 1971 | 5 | 172670879 | GGATTCCGAGGATCCGAGGGAGTCGCTTTCAGACTTCCGGTGTCCTTTCG |
| 1972 | X | 101906035 | CGGTTTGGTATCCATCCGCTGCTCCCTCCAGCGCGGCTCTTCGCGACCTC |
| 1973 | 11 | 61128572 | TTGTATCCTAAATCTGGTCCTCAAGATTCTTGAAGTCCCCCAGTCCATCG |
| 1974 | 17 | 33700759 | CGCGGCAGAGTTCGCGGGCTTAGCAGACCCGCCCATTGGCTCTTGCATCT |
| 1975 | 4 | 8396163 | TGGAACCGGGAGTCGTGTCAGCACATCCTTGAGAGGCAGGGGTGTGGACG |
| 1976 | 6 | 133453019 | CGCAGATGAGTTAGAGACACAGAGAAGTGTACATCCTTCAGCCTTTCCCG |
| 1977 | 14 | 91580605 | GCTGTGCTCTGTTCCCTTCTGACTTACTGTTTGTTTGGGGCGTCTGGACG |
| 1978 | 19 | 46917018 | GGAACCGCTGCCTTTCAGGAAAGGTGCTGACGTGGGCCACTGCCGCTTCG |
| 1979 | 5 | 140625004 | CGGACCAGTGTCTCCGGAGAATGCTATTCTCCTACATTTCCGAACAGGTT |
| 1980 | 10 | 105036611 | CGGATGGGAAACCTCAGTCGTTTAGGCACCCCTCCGCTCGAGTCACTTCC |
| 1981 | 13 | 29293279 | AGTGGCTAGTGGATCGTGCACCCACCCTCGGAGGCCCGGAGTGGATCCCG |
| 1982 | 6 | 142409417 | CGGAAGTGAGCTGGATGACAGGGATCAGTTTGCAGCCCACCTTGCCAAAC |
| 1983 | 3 | 79817278 | GGCATTACAATACCAGAGGTAACGAATCAATTAAATCCATTTTCCCCTCG |
| 1984 | 19 | 46999274 | CGCTGTGGCCTGAGACGCTTTTCTTAAAGGTCCCGATGACAAGGACTTGG |
| 1985 | 15 | 89951787 | AACTCTGAAACCTGAGGGCCTCTGCCGGAAGCTGCTGTTCGGCCCATCCG |
| 1986 | 10 | 105428506 | AGAGAGAACGGTTAGGCAGGCAGGAGGTCCACAGTGAGTGGCTGCAGGCG |
| 1987 | 17 | 76102450 | CGACAGAGTCCACGACGATGGCCGAGGGAACAACACTGTCCCGTCCCAGA |
| 1988 | 13 | 36919905 | TAAAGAGTAAAATCACGCGCAATCCATCCCAATATAACCGCAGTTGTTCG |
| 1989 | 1 | 153029939 | AAACCCCTGGTACCTGAGCACTGATCTGCCTTGGAGAACCTGGTGAGTCG |
| 1990 | 7 | 137028617 | CGGATTTGAACCAAGAGGCCATGAGATGGTACCTTGTTTTGTTTTTAGCA |
| 1991 | 6 | 100895050 | GCTCTCTGCGAGAGTGCGGGTGGCGGCACGGCCGGGTTAGACAAGGCTCG |
| 1992 | 2 | 157178731 | AGAGAGATATGTAAGGCCCAGGGAGCCGATGGCGCCCTGCAGTGACAGCG |
| 1993 | 1 | 110610899 | GCTCACTCGTGTGGACTGGAAACGCTCCGAGCCGGTTATTTTAAAAACCG |
| 1994 | 3 | 16216100 | TGCAAAGCCTGGGAACAGTGAGGGCATTAGGACCAGGAGTGACGCGCACG |
| 1995 | 4 | 17783331 | TGGGGGGTTTGCGGTGGGCGGGCACTGCGCTCTTGGACACGTGGGTGGCG |
| 1996 | 7 | 19146324 | ACCCAACGGAGGGTTTATTCTGCCCAGCTTCACATCTGGGGGTCAAATCG |
| 1997 | 1 | 215256262 | CGCCCCGGCGTCTTTGTGCGCGAGCCTCGTTCAGCCCCTCTTCGCATGTC |
| 1998 | 14 | 105103075 | CGCCTGGGGCCTGAAGCTCGTGGAATCACCCGCCTCACGTGTCTTTGCGG |
| 1999 | 1 | 237168540 | ACGAGAAGGTGCCACTCAAACCTGAGTCAAGGAACACAGCTAAGTCCTCG |
| 2000 | 14 | 23834977 | AGGGGTGCCTGCGTGCTGGTTGCTGCTGCCCGGGGCGAGGCGCCGTAGCG |
| 2001 | 11 | 91957989 | GCGTCGGCTGTGATGGGGAGCCCGGAGCAGCCAGGCGGCGAGCAGTAGCG |
| 2002 | 14 | 48145000 | AAGGGGTTCCCCGCTTGAGCTAACGTAGTGGTGGCATCCAGAGGTGAACG |
| 2003 | 6 | 43276478 | CGGGCCTCACCGAAGAAAACAGGTTGCTGGCAACGCGGACAGGTCCAGCT |
| 2004 | 13 | 110522265 | CGGCCAGGAGACCCTCCCTGTGCAGGCAATGCCGGGTGAGGCATCGCTCC |
| 2005 | 20 | 52276947 | TTACTCGAGAATCAAAATAAAGAGGCGAGCCAGCGTTCCAGAAAATCACG |
| 2006 | 6 | 50792868 | CGGAATTACCTGATTTGTGCCTGGATTTACTGATAGTAATAAGAGTGATG |
| 2007 | 6 | 76059576 | CGGGGTCAGCCTCCTGCCAGCTCTCATAAGTAGATCTAATTATGACCGCT |
| 2008 | 5 | 87974446 | GGTGGCTCTTGGTGCAGGGCATGCTGCTCTCGGAGGAGTTGGGGTCTGCG |
| 2009 | 14 | 97499987 | CGGGCCTGGGTTTAGGGTCCACAGCTCTTGCCAAATTCCAGAGGCTGGAA |
| 2010 | 6 | 29795815 | CGCCCCCAGGCTCCCACTCCATGAGGTATTTCAGCGCCGCCGTGTCCCGG |
| 2011 | 7 | 141438973 | CGAAGACCTGTATTACAGGTAGTCACTTGTCTGTATTAATACTGAGATGT |
| 2012 | 11 | 32455362 | CGCTGTGCTTAGTCAGGATACAAGGAGGGAACTCGGCACACGATAAGTAT |
| 2013 | 17 | 34819740 | CTCACTGGAGGGGACGGGTCTGGAGCAGGCAGAGTGAGCCGCTGCTTGCG |
| 2014 | 13 | 97646803 | TTTGGAACCTCTGCAAACACTTTCTCCTGGGTCAGTTTCCCGCCAAACCG |
| 2015 | 11 | 133256591 | TGTTTTCCCTCTGCGATGTCCTCTTTTAATTCACTAGGTCAGGAAAAGCG |
| 2016 | X | 136509451 | CAGGATCCGGAGGCAGCGCCCTGTGCAACTTTGGCAACTCGGAGGACGCG |
| 2017 | 5 | 273394 | CAGTATTCGCACACTGATAAGAACACCTCCCCATAATCCCCTGGCCAGCG |
| 2018 | X | 39864984 | CGGCGTGTGAGCTTTGTCCTTTGGTGGGGCTCCTGGCCACGGGCCGAACG |
| 2019 | 4 | 57976756 | CAGGGGACTGGGGACCGAGCAGGAGCTGGCGGGGTGAGCTCTGCGAAGCG |
| 2020 | 2 | 223166909 | CGCTCCACTCAGGCCTGGACAGCCAGCCCGCTGTTATTTTCACCTGTAGT |
| 2021 | 3 | 47425198 | ATGGAGGGAAGTCACTGGTCACACAATACCCTTTGGGTCGGTGTGAATCG |
| 2022 | 2 | 178257923 | TGGGCGGAAGGCATCCCGGTCCCTGAAAGTAGGGACCCACCTCTCTTTCG |
| 2023 | 6 | 101849938 | ACCTCGCAGGAGGTGACAGGAGGACGCTGGGTCGAATCACAGTCCTAGCG |
| 2024 | 15 | 96898476 | TTGTGACATAGTTGTAATACAAACTCAGCCGCCTTGCACTGCATCTTGCG |
| 2025 | 6 | 30292899 | CGCAAGTAGTATGACATATAACAGGGCTTGGCAAATCTTTGCTGTAGAGA |
| 2026 | 3 | 128446370 | CAAGAGCTCCTAATTCGGTGACACTACACCACAATATGTGTGATAAGTCG |
| 2027 | 6 | 114891274 | CGAAAACACAAATAGTAAGTGGTAAGAAGGGCTGAAGTTACTACCTACTG |
| 2028 | 6 | 26250919 | CACAAGTCCTATCACACTACATAATTCAGGGCGCGAAACTGGAAGATTCG |
| 2029 | 19 | 1083193 | CGTGGAGGTGGAGCAGGACAACAAGATGACCCCCGGGAACCTGGGCATCG |
| 2030 | 14 | 91580380 | CGGCAGCTAGCCCGCCCCAGTTCTTAAAGCTGCAGAGCTGCTGGCAGCTG |
| 2031 | 19 | 57646138 | TTGAAGGTAGCGAGGACTTATTATCACAGTATCCTTTGAAAACCCATTCG |
| 2032 | 15 | 100913948 | CGCTTTGGGGACTGCGTTTTATCCACGATAAACCATTTCCCGAGAAAGGG |
| 2033 | 2 | 200334137 | GCAGGAGTAGTGAGCGATGCCGGGGCTGGCTTTCCTAGATTTAGGAACCG |
| 2034 | 17 | 36105364 | GGCTCTCACCTGAACCCCGAAGCGTAGTGTCTTCTCTCTGGACTAAAGCG |
| 2035 | 1 | 50889427 | AGTTTCCGTGCGCCCGAGAGCGTGTGAATTGCTCCTAATTACCGTGTCCG |
| 2036 | 1 | 1602481 | CGCGTGGCAGGCTCCCAGAGGAGAGGGCCCTGTTATTTACGTCTTTGTCA |
| 2037 | 2 | 38892846 | TTCTTACCCCCTGTATCAAAGAGTTAAAATCTAATTTTTTCCCAAGATCG |
| 2038 | 12 | 48397225 | CGATAACATTCCAACCAGACCGTGCTTTTCAAATGCCCCCGAAAATAGCG |
| 2039 | X | 139593443 | CGGGTCTGCCTGAGGATAGGCATCCTCCATACCTGGCAAGCAGCAAGCGG |
| 2040 | 2 | 20865674 | TTTCCTTTAAAATTGCATTTTCTGCAGAACTCAAAATCTAGACTGGGTCG |
| 2041 | 3 | 142666476 | CCGATGCAGGCTCAGATATGCTGCCTGCGGGATGTCGTGCTTTCCAGGCG |
| 2042 | 18 | 67070120 | GTAGAAGAAAGCGCCTCATTCTTTTCCAGGCATATAAGGACGAACCCACG |
| 2043 | 1 | 59612408 | CGACTTCTTTCTACAGTTACTAAGCGCCCACTGTGTGCCCTGGAAGGGAC |
| 2044 | 2 | 187713876 | TTCAGAGGCCTTATAGCCTCGGCGAAGCATGCTGGGTGCGGGCGGAGGCG |
| 2045 | 12 | 58145283 | GTGGCTTTACTGAGGCGACTGGAGGCTTTTGAGCATCCCAATGTTGTCCG |
| 2046 | 11 | 124043067 | CGAGGGACTGTTAACCATGACCGTCCTCATAGATTAAATTAGGTACTTGC |
| 2047 | 21 | 22370046 | CGCCGCGACAGAGGGGAAGCAATTGTACCCTAAAGTCTACACTGTCTGTC |
| 2048 | 14 | 38067622 | CAAGTGGGGGATCCGCGGCAGTGAGGACTGTAGGGTGCGCGGGTACTCCG |
| 2049 | 6 | 28129416 | TGAGGTAGGAAGTCTGAGCATTGCCGACGGGCCGGAGCAAATCGCTCCCG |
| 2050 | 4 | 81106312 | GGTTGTGATTAATTTTTCCGAGGGGATTTGGGCCGGGAGCTTACGGAACG |
| 2051 | 17 | 36728859 | CGCCCCCCGCCCAACCCGAGGCGGCGATCCCGGCCCCCACAGTCGCTCCC |
| 2052 | 12 | 50444723 | AGTGCCGGTGAATAATTCATGGGGCTGGGAGGCCCTTATCTCCCTGGCCG |
| 2053 | 2 | 19499118 | CGCTGCCAGAGTATTTGTGGGAAGAGAAAGAGGAGGTTGGATGAATCACT |
| 2054 | 11 | 116371330 | CGCTCCCCCCACGAGCCGCCGCACTCCCTAAATGAATGAACTGGCTGCTC |
| 2055 | 1 | 145415412 | CGGCCTCTGTCGAGCCCTCCGCTCCTATGCGCTCTGCACTCGGCGCACCG |
| 2056 | 6 | 42145954 | CGTCTCTTGGGGCTTGCCGAGATGGGCGGAGCCTTGGGTTATGATGGGCG |
| 2057 | 10 | 30405328 | ACTGACTGGTTGCATCTTGGCCAACTGCTCCTAGGCAGGGCTGCATTTCG |
| 2058 | 14 | 48144369 | CGGGGACCTTCGCGAGTTACTTTGTAAAGGCGAATCCAGGCAAGAGAGCC |
| 2059 | 1 | 20512463 | CGGCCTCCGCCCTCCTGGCCCAGCGGCCCCCACCCGACCCCACCCGACCC |
| 2060 | 6 | 108487820 | ATGCTTAAATTTCCACTGTTGGACGAATTCTGAGCGCCCAGGGAGCAGCG |
| 2061 | 11 | 118402540 | CGCCCTCCGGCACACACTGCTGCTCCTGCCAGCCCTTCTGAGCTCAGGTA |
| 2062 | 10 | 105428651 | AGGGAGGATCGTGTGGGATTCTCTGTGCTCCGAAGGAGTCCTGCATTTCG |
| 2063 | X | 20134778 | CGCTCGAGGACCTTTGCCCTGGGCCGCCGGGCCCGCCCCCCCACAGGAAC |
| 2064 | 11 | 132735814 | GCTGACCACCAGGAATAAACAGATTCCTGGCAATAGCCATTATTAGATCG |
| 2065 | 4 | 174440103 | CGAAGGGCCCCGAAGGCAACCCGGTGGGTAGGGGCGCGGCGGACAGGACG |
| 2066 | 9 | 135462522 | GACCTCTGGGGCCCCGGGAACAGGACATGAACCCTGAGGGGAACAGGACG |
| 2067 | 1 | 46951354 | CAGGAGATGGCTCGTGGCAAATTGCTTTGTTAGTCTTGTCAGCGCGGCCG |
| 2068 | 1 | 50892674 | GAGGCATTGACTTTTGCGCTGTTTGCCCGCGGCCTCGGAGCAAGCAGACG |
| 2069 | 1 | 91184501 | TACACTCCAACGAGGCGTTACAGTCATGCTCCGGGTCCCAAGTGGATCCG |
| 2070 | 3 | 50373641 | CGCCAAAATCTTAGCCCAGAGCTTGGGTGCAAGGGTTGAGTTGAGTGTAG |
| 2071 | 10 | 119304705 | AACAAATAATTTAAAAATAAATCTCCATGAAAACTCTGGAGAGCAGCCCG |
| 2072 | 6 | 26273145 | CGAAGTGATACCGTCAAATGACCACAGGAAAATGCTGGTGAGCCCAAACG |
| 2073 | X | 136632433 | GCGGGATGAGCTGCTTTCCAAAAAGGATCTCGGTGATTGGACCGAGCTCG |
| 2074 | 7 | 54609776 | TGGCTCGGCGGATCAGATCGCTCGGTGCCGAGGAGGACAATACAGTGACG |
| 2075 | 14 | 95239570 | CGCAAATTGTCCACCTCGTGTTGCTAAGCGATTGTTTGTCGGCCCCGCAC |
| 2076 | 10 | 73846858 | CGGGCCCATCCCCCACAGCACCCCCAACGATCTCGGGGTCCAGCCACACC |
| 2077 | 12 | 115136200 | CGCGCCCCAAGAGCCTCTGCCAGGCCGCTGGGACTTGATTCATTTCTTGA |
| 2078 | 8 | 26047996 | CAGCCTGTAGAAATCCGTGCTTTGCTTCCTGCACGGCGGGTAGAGCAGCG |
| 2079 | 7 | 28998195 | CGGACGCAGCCCGCGTGCTCCCCGAGCTATTGCACTGGAGCCGCCCTCCC |
| 2080 | X | 151122266 | CGGCTTTGCAACAGGCATTACTCGCCATTGATTGGTGCCCACCCAGGGCA |
| 2081 | 15 | 100913842 | GGCCGAGGCCGCGGCTGTCGGGGTGGCCTGGACGGGTGCAGGGCTGGGCG |
| 2082 | 17 | 48546431 | AGAGCGGGGGGCTACAGCCGGGGCTCAGGGCTCGGGGCTCGGGGGCCACG |
| 2083 | 6 | 88877216 | TCTCCCGATCCAACTCACGAGAGATAAAGATGTCTAAATAACCACGACCG |
| 2084 | 4 | 122301740 | CGCGGGAGCAGTTCATCGCTCTGTACCGGCTGCGACCGCTCGTCTACACC |
| 2085 | 4 | 3371876 | AACCCTGGCAAGCCACAGCTTCCTGCAGTGTCCTGTTGCCATGGGTTACG |
| 2086 | 21 | 38807712 | TAAAATCCGCCTGCCCGCAGTGAGTGGGAACTGCTGCAGATCATTAAACG |
| 2087 | 14 | 24641077 | CGGAGCCCATTGGTCGGAACACCTCACAATGGACCCCAGCGGCGCGCAAA |
| 2088 | 15 | 55610624 | CGGCCCACTCCTCTTGTTAAAAGTAAAACCAGGCCGGGCGCGGTGGCTCA |
| 2089 | 2 | 208492057 | CCTCGAAGCCACAGGCCCCACCTTCCGATGGAAGATTAATCTGGAGGCCG |
| 2090 | 14 | 36978633 | GCCATTTGAGCGTGTTTCAGCCAGATAATTACGTTGCCATTGTGTTACCG |
| 2091 | 6 | 162831837 | CGCTTTTCAGCTCCTCAGGTGATAACAGAAGCCAAACTCCAAGATAGTGG |
| 2092 | 1 | 91192803 | TCACTCCATGCCATGAGAGAGAACCTGGCAGTGTCAGATTCACTCCAGCG |
| 2093 | 12 | 127211421 | CCTGGCACAAAGGGCCACGGGCGCGAGGGACTCGATGAGCTGGGAGATCG |
| 2094 | 2 | 100686693 | CGTGGTTGCGTAAGCACATCTAAAGGTTAAACTCCAAGCAGCTAATCTGC |
| 2095 | 3 | 62356962 | CGCATATCCGCATCCACGCGGGCTACAAGCCCTTCGTCTGCGAATTTTGC |
| 2096 | X | 51138790 | AGCCATGAACATAAATCCGGCCAAGTCGTGGCCCCGCAGCGTGGGCTGCG |
| 2097 | 7 | 29186234 | AGCGCTGACTTGATGCGGCCAGGTTTCTGGGACCAGGAGGTTGGGACACG |
| 2098 | 18 | 67070045 | CGGCCAGGTTTCAAGGGCTTTTCTTGGGTAGAAGAAAGCGCCTCATTCTT |
| 2099 | 20 | 53092334 | CGCCTTCTAAAGCCTCGCCCAGCGCCGCCGAAGCAGCTTCACCTCTCCAA |
| 2100 | 10 | 102896869 | CGCCCCCCTCCCAGAGGCGTCCCGCACCTGTCTGAACTGTTAAGAAATCT |
| 2101 | 17 | 13972099 | AATTACACTTTACCGTAGTCTCTATCAAATGGGAGCCCAGAGGAACTCCG |
| 2102 | 4 | 13536910 | TGTTTCGGAAAGTCCAGGTTTTCCTCGGGGAGGACTTCAAACTCACTGCG |
| 2103 | X | 118407296 | CGGATCCTGGAGTAGAGACGAACGGTTCCTGAAGCTCCCGGTAAATGCCC |
| 2104 | 19 | 35395800 | ACAGGGAAGGTGCCTGGGGAGGGGCCGGGCGGTGCTGTCTCCTGACTCCG |
| 2105 | 2 | 21022587 | ACTTAGGGGTTCAAATGCTGCATTACCGCGCTCGGGTGAGCGACTGCCCG |
| 2106 | 7 | 158800888 | CGCTAACAAGCATTGGAATCGCAGGCTCAGAGCCAGATGGCCTTAGAGAC |
| 2107 | 6 | 28367749 | AGGGATTTCCCTGTCTTAAGATCAGTTCTACAAGCCTCGACCACCCATCG |
| 2108 | 1 | 43423072 | CGCCACCCCGCTCAGTCTCAGCGGGACACCTCCACAGCCAGTAGGAAGGG |
| 2109 | 4 | 72897868 | GCGAAAAGTAGCTGGAGCCGGAGCAGGGACAGAACCTGTTGCTGCAGACG |
| 2110 | 1 | 86048540 | CGGCCGTCCACGCAGGAACCGCAGTACTTGGGCCGGTATTTCTTCACACT |
| 2111 | 6 | 108440280 | CGGGGCCACTTTGACCGCCAATAGTGGGGAAGTCAGAGCTAATCCTCAGA |
| 2112 | 14 | 54422775 | AGCAGGTTCTGCGGTAGAGAGCGCAGTCCTGCTGGCCTCTGGAGAGTGCG |
| 2113 | 7 | 39170763 | CGGGAATCTGAGCCGTGCCAATCGACGTGGGGAAAATTTCACAGCTGCTG |
| 2114 | 6 | 133561932 | CGTGAAATAAAAACCAACCCAGACCAGACTGGGGCGCTTTTTCTGACGGG |
| 2115 | 7 | 39015705 | CGCCGGGGTGCGCTCCAGGCTTGGCTGCAAACCCCCCAAGAATTAGAAAG |
| 2116 | 12 | 54391631 | CACTTGGGCGGCTGCTGGCAGCGATGAGCAGAGACGTTTAGCCCTCAGCG |
| 2117 | 6 | 16986186 | CTAACCACTCTGCCTAAGCAGCTATGACAGTGCATCCCACAGTTAAAACG |
| 2118 | 17 | 58216297 | GGGCCTTGAAAGCGAACTCAGGAGATGGGGCGTTGTGTATAGGCAAGTCG |
| 2119 | 13 | 97646778 | CGGGACCTCTGGACATCCTCCATCCCGGTTTGGCGGGAAACTGACCCAGG |
| 2120 | 21 | 47703630 | CGACAGGACCATTCTGGAGAACCATTTTGGCAAAATTGCTAAAGTGCAGC |
| 2121 | 5 | 49736085 | AGTTGGTTATGATGTTTTTTCCCCTACTCAGCTCTAAAAAAACATCCTCG |
| 2122 | 20 | 26190347 | CTTCCTCGCCTGCGGGTGGTGCGTTGGGAAGGCATGGGGTGCGGAACCCG |
| 2123 | 21 | 47717575 | GGGCCTCTTCGGAGGGAGCTGAGGGCCGCGTTCCTTCTGAAAGCGGGACG |
| 2124 | 12 | 25055967 | CCATGGGGAGGCTGCGGGGACGCGGGGCTGCAGAGAGCGGCAGTGGCACG |
| 2125 | 1 | 26527928 | CGACCCGCTCGTCGACGAGCGCCTGTTCAACACCTCTGACTCCTGCTCCT |
| 2126 | 2 | 202122474 | CGCCGGACTGGGTAAAGCCAGCCCTGTAGCTGAAATGTAGGTAGATTGTT |
| 2127 | 1 | 84763338 | CGGACTTCCTTGAAATGACAAACAAGTAAATATTTAAGGGAATAAGTAAA |
| 2128 | 5 | 178288359 | TAGTTTGACAAGTAAGATACCACAAGTCTGTCTGTCACTAAGAGCAAACG |
| 2129 | 2 | 107103716 | CGCGGTCCCCAACACCAGACTCCCGCAGCCACCGCGCCCGGTCCCGCCCT |
| 2130 | X | 150797951 | ATAAAGCACATTCAGAGACTGGCTAGAAGTCTTAAGCTTGGTCGTGCACG |
| 2131 | 3 | 170136920 | GCAGGTGGTGGGCTTCGTCACGAGCTTCGTGGGCTGGATCGGGGTCATCG |
| 2132 | 8 | 68178729 | CGAAAACAATTCCTCATGAGGAGTGAGCTGAGAGCAGAAATCCAGTGGAT |
| 2133 | 6 | 19692011 | TCATTCAGCCTTAGGGGGGCATTTCTGCATTCTTTTCCAACAAGATGACG |
| 2134 | 10 | 3149673 | TCTACGGGCTCGGCAAGAGGTGACGTAAGCGGAATGGGCTCCCAGGAGCG |
| 2135 | 17 | 37321631 | CGCCCCGGGCCTTTCAGACACTTTTCTGGAATGTGAAGGGAGGTGGGGGC |
| 2136 | 8 | 79710803 | TACAATTACGTGCAGTACATCATAAAATTCCAACAATATGTAACTCTTCG |
| 2137 | 20 | 57090013 | CGCGCACACCCGCGCAGCGCGCACAGCCCCCTGGTGCAGCTCCTGCCATT |
| 2138 | 18 | 31020973 | AGGAGTGGAGACGAGTCAACGCCCCTTCGTACAGCAGTGAGGTTAGAACG |
| 2139 | X | 70151134 | CGCAGTCTCAGGCCCCACGCAATCCTTTTGCAAAGCCAAGAAGTGTGTGT |
| 2140 | 2 | 46526843 | CGCAGGAGGAGCTGACACTTCACACATTTGTCAAGATTACTGTCAGGCCC |
| 2141 | 6 | 129204206 | CGCCTTCCATGGGCACGCGGCCCCAGGACGGCCCTCTATTAAAGGATCCT |
| 2142 | 2 | 228736258 | CGCCACCAACCGCCCGCGGAGCCAGACCTCCCAGCTCTGCGCCCCAGGAC |
| 2143 | 13 | 74707145 | ACGGGGGACCTGGCCGGAGTTGACAAGGGGCAACTTTGTACGTCCACCCG |
| 2144 | 5 | 153192843 | TTATTTCCAACTCTTCCAAGATGCAGACTCCCTAATGGTGGGTCACATCG |
| 2145 | 11 | 64883528 | CGGCTCCACCACCCCAGGTGGGGGCATGTGCCCACTCATCCACCAGCACA |
| 2146 | 13 | 36919674 | CGCAAATTCGTTAAATTATGATACAATAAGGCCAACCTCGAGGATCCTCG |
| 2147 | 8 | 145895604 | CGCTCTAGCATGCGTCCGAGCTCTGTTGCTTTTTACAGCCGCCTGGTCCC |
| 2148 | 6 | 129204125 | CGGTCTCAGAGGGAAGGAGCGCTCTAGGAAAGGAGGATCCTTTAATAGAG |
| 2149 | 1 | 197887714 | CGCCAGGACCCGGCGGGTGTCATGTGTAGGGGGAAATCAAATAAAACATA |
| 2150 | 5 | 145720024 | CGCTGCGCCCTGCCGCAATCAGTGGACAGCCGAATACTTCATTCGTTTCT |
| 2151 | 8 | 105479420 | CGCCCTGAGTTGAGGTATCCACCCAGGCTTCGGAGTCATTGGAAGGGAAA |
| 2152 | 11 | 830320 | GAGGTGCGCGCTCTGTACGAGGAGACGGAGCAGCTTCGGGAGCAGAGCCG |
| 2153 | 6 | 19692082 | CAGAAATGCCCCCCTAAGGCTGAATGAGCACCACTTCCACACTCAGGGCG |
| 2154 | 7 | 157476887 | CCGGTCTTGACTGGAAAAGCTGGTCATTAATTAGCCTCCTTTTGCCTTCG |
| 2155 | 11 | 76859010 | GGCGAGGTGGGCGGGGCGGGGCCTGAGGGGGCCTGGACGGGCACCGGGCG |
| 2156 | 7 | 150652948 | GAGGGGGCCGCTGGCTGGGCACCGGCGGGGCGGGGGAGCCTAGTGTGGCG |
| 2157 | 12 | 112205368 | AGCAATGGAAGCAGCCGAGCGTGGCCTCGCCCATGGTGAACTGGAGAACG |
| 2158 | 3 | 38035831 | CGTCCAGCGACCTGCACTACTGGGTCGGGAAGCAGGCGGGTGCGGAAGCG |
| 2159 | 4 | 41875606 | CGGCCACTGAAAGCCTCCCTGTTCCTGGGCAGGGCAGCCAGAGTGCCCTC |
| 2160 | 3 | 131002896 | CGGACACCTGTAAGTGTGATTTACAAGACTGAGGGCAAATGCCAGGAGCC |
| 2161 | 14 | 36979758 | ACATAGGCATATCCAGTTCCTTAGACATTGACTGGGTCTCCTGGGTTGCG |
| 2162 | 8 | 11551964 | GCTGGCGTGGCCCTCAGGGATCCTTTCTTTTCTTCCCCCAGAACATTGCG |
| 2163 | 3 | 10184584 | CGCCACACACGGTCGGCTCTTCCGGCGCCAACATGACCCTGGCTTGTTCC |
| 2164 | 14 | 36991034 | TGCTTAGCCACCACAGTGATCAATGCTCAGTTCGTCGGGGCCTGAGAGCG |
| 2165 | 14 | 60386396 | CGGGGTTCGTCCCGGCTGTCACGCCACCTTGTGACATCACAGGGCCATGC |
| 2166 | 3 | 147109784 | CGCTCGCCAGTAATAATATTTCATTACGCTGCTCCAGAGGCTTCCTGGAG |
| 2167 | 14 | 20898781 | CGTCCGCAGAGATACCTCTGTGCCCTGGGATTCCCTCATCCCGCTCAGGA |
| 2168 | 19 | 17357587 | TTTTAATTTCCTTTTCTTTCTGTGCTTTGCAGAAAGGGCTTGGCCTGGCG |
| 2169 | 11 | 65409861 | CGCGGGATCAGGACGTGGGTTGCCTCCCCGACACCTTGTATGCATCCTGA |
| 2170 | 11 | 18067927 | CGCCTGCTCCCAGAGACTTGCACGCCTCAATCTCTGCGTGTATCTGACTG |
| 2171 | 11 | 67250046 | CGGAAATGAGTCATAGTTGTGCCCAATTAATATTAGGGAATAATCGATGA |
| 2172 | 11 | 44340839 | TCCCTGCCAGTCACAAGCCTCGGGATTATGCGATCAGAATGTGCGCTCCG |
| 2173 | 7 | 39649254 | CGGGGCTCTCAAGCCCAGTAGGCTCCAATCCGTTGAGTTCCTGTTTTGAT |
| 2174 | 6 | 43276647 | CAAGTGGAGTGGTTCTAAATTTCTGATAACCAATGAGCGCCGAGGAACCG |
| 2175 | 2 | 220325369 | AAAGGCAGATCCCGCGGCTTGGGAGTGAGCAGAGTGGCTAAATCCAGACG |
| 2176 | 3 | 173302303 | ATGAATTCCCGGTGCTGATGTCCAGAAGAACAATTTCATAACCCTGTTCG |
| 2177 | 7 | 64035529 | CGGTAGTCAAGGGAAAATCCTGACTCAGGGTGTGGGGTTCATGAATGAGA |
| 2178 | 8 | 126450475 | TGCCAGCCCAGAGTCCTTAGTCGGATAATGTATCACAGATACAACAGTCG |
| 2179 | 22 | 46262436 | GGAGCTCGGCCCCGCGCGGGGCAGAGGGAGTGGTCCAGGGCTCGGGAGCG |
| 2180 | 17 | 58216415 | CAAGCGCGGCCTCGGAACCGAATGGACAAAGACGACGGGGGCTGGAGACG |
| 2181 | 9 | 138393651 | CGGTGGCAGCAGAGACAGGCAGAGCGCAGTTCCCGCCCACCCCTGCTCCC |
| 2182 | 8 | 73163868 | ATCTGGTGCATCTGCTCCCCACGAGCGCGGGCACCTGGGGCGAGAGAGCG |
| 2183 | 18 | 22929205 | GGAAGAGGTTTGTCCTGGTACTTCGCTCAGCGTTACTTTCAAGAAAGCCG |
| 2184 | 6 | 27521385 | CGGGTCAATTCAGAAACCAGCTCCCCTGGGGGCAAGTTTGGCTCTGTGCT |
| 2185 | 22 | 36044151 | GCTGTCATCACTGATTGTTCTGAAACTGCTTTCCTCCACAGCATCTTACG |
| 2186 | 17 | 41363774 | CGCTGCCAGCTCTTCAAACGTTACCCGAGGGCCCGGACGCTGCTTCCGCA |
| 2187 | 9 | 139290292 | CATGTGACGAGATGAGACGTGGATGCTCCCCTGTCCTCGGCAGCCAGTCG |
| 2188 | 15 | 60288404 | AAGGTAGGCTGTCTTTTCTTCCCTCGCCGGCAGTAAAGGAAACCTTCTCG |
| 2189 | 14 | 38677095 | CGTGCCCAAACCATTAGGAGATATCAATGTAGGCAAGCCTGAAGCTCACT |
| 2190 | 21 | 42620091 | TTTTGCGTCTGGATTTCCTCCGTAGAGAGGCGCTGGGCCTGAGCCAAGCG |
| 2191 | 6 | 100615670 | TGACCTTCCCTCTAAAAGGTAACACGTAGAGATTCCTAAAGGAACCATCG |
| 2192 | 1 | 159558512 | CGAGAGCACTTGAAAATGAAATGACTGTCTAAGAGATCTGGTCAAAGCAA |
| 2193 | 18 | 14748230 | CGGCCTCTGTCGGGAGAAATTCTGAGCAGAGCCGTTAGCTGCTCTAACGT |
| 2194 | 2 | 188312833 | CGCAGAAGAGATTTACCCACAAGCAAGGTGGGAAAGAGTGAAAGCTTTGC |
| 2195 | 12 | 53208752 | ACAGCAAATGGAGCAGCAGAGTGCACACCACCAGACCCCGCTGGAGAACG |
| 2196 | 12 | 94083470 | CTCAGCACCATAGCAGACAGGGTGGCAATACCATTACCAAAATAATACCG |
| 2197 | X | 144903565 | CGGGCAGCTCGCCCGTCTTGAGCTCCCAGCAGAAATCGCAATCGCTTTAG |
| 2198 | 10 | 22624094 | GAATCTCAAGATCTGGGCAAAGGCTCGCGTCTCGGGACGCGAAGTCGACG |
| 2199 | 1 | 119530677 | CGCCCGAGTCCTGCTTCCCACCCACCGGGGCAGGCGCTCACAGACTGTGT |
| 2200 | 1 | 203598954 | CGGGCTGTGCTCCTTTGACTTGACCTTGAATCTAGAGACTTGGTGGTAAA |
| 2201 | 7 | 117119938 | GGTGGAGAAAGCCGCTAGAGCAAATTTGGGGCCGGACCAGGCAGCACTCG |
| 2202 | 3 | 65506902 | TGTTAGAGATACTACTCACATCCCACTTCCACTTTATTACAGAGATTACG |
| 2203 | 17 | 46691543 | AGGCCTGGCAGGCCGGGGCGCATTTCCTCCAGCTTCTGGAAGACTTGCCG |
| 2204 | 12 | 22094699 | AGTGCACGAATGTCTCACAGGAGCAAGGCTGGCTCAAGTCTAATTTTCCG |
| 2205 | 22 | 19754448 | CTGGGGCTGGGGGAACGTATTCCTTGCTTGCCCTTGGGGCAGGGTGGCCG |
| 2206 | 1 | 76540574 | GGTGGCAGGAGGGCCGGCGGAGCGCCATGGCCTGCATCCTGAAGGTAACG |
| 2207 | 7 | 151107400 | CGCCGGGAACTCATTTGGGGCGCAGCTCCACCGACCCCTCGGCTACTCGC |
| 2208 | 12 | 53208709 | CGGGGCAGCATGGGTACCCAGGGCTGAGAAAGACAGTCACGGTTTTCTTT |
| 2209 | 19 | 47730821 | CGCCTGCCGGCAGGGATGGGGCTCCACGGGCACCACAGGGTTAACAGCCC |
| 2210 | 3 | 138658704 | CGCCCACAGGCTGACCCTGCAACCTCTGGCCCGGTGGCTACCTTCTGCTT |
| 2211 | 6 | 26600049 | TCTATCACTTCATGCAACAGCCTGGAAAATGATCAGAATTTTGGTCTACG |
| 2212 | 12 | 121454837 | TTGGTAACACCTGGGAGCTCGTTAGAAATGCAGAATCCAGGCTGGGCACG |
| 2213 | X | 150396241 | TGGGTGGGTTTAACTCAGCTAGCAGCTGCGGCGGTCTTGGCGCGCTCTCG |
| 2214 | 2 | 175548020 | TTCTTGCGGCTGCTGGCGGGGGGATCGGGTGGGGGTGTTCCCTCTGTTCG |
| 2215 | 11 | 20184350 | CGACGGACAGGAAACGGTGAAAACTACACCCCGGCAAATGCCCCAACAGG |
| 2216 | 2 | 17699740 | CCAGGTCTTGAGGGGCGGGTCAGGGGCGTGACCAGAGCGGCCCGGAACCG |
| 2217 | X | 134847724 | TCTGTCTCGGTGATGCTAGGGACTTTTCCTACTGAAATGACCTGAGCCCG |
| 2218 | 6 | 50794057 | GGGCTGTGGCCCTGAGTGTGGTGCATCTGGGTCACACAACAAAACCCTCG |
| 2219 | 2 | 85641076 | CGGCGACCGGACCCCGAAGCGCACTACTTGCCTGCCAACGCCTTGCTCCG |
| 2220 | 6 | 26250936 | CGCCCTGAATTATGTAGTGTGATAGGACTTGTGATCAGCTACTAGGTCTC |
| 2221 | 6 | 55444061 | GCGGGGAGTGGGGCGTGGCAGGTGGCCGCGCCTGGGGCAGCGGGTGCACG |
| 2222 | 12 | 54345862 | ATTTTTGTCCGAGCCCGGTTAAAAAACGGGTGCCTGAGTTGCGTGTGTCG |
| 2223 | 14 | 38067742 | CGCTTGGCTGCCTCAGCTTACACTTCCCAGCAGGATCTGGCTGGGAGCCC |
| 2224 | 7 | 142494439 | CGCTCTCCCAGCACCCAGAACCAGGAGTCCTCCGCCCAGACGCCCAGCAG |
| 2225 | X | 130929940 | TGGCCCCTGTCGAGTGCAGGGCCAAGGCTTGTGTTCAATCGTGTGAATCG |
| 2226 | 11 | 89867704 | CGGGCAACTTCCATTCTGTGGATCCCGCAGTAACCCGGACATAGTAGGTG |
| 2227 | 1 | 13910569 | CGCGCTTCCCAAAACGAAGAGCAGAGCTGACACCTTCCACATCGTTCCCG |
| 2228 | 2 | 176969223 | TTAAGGTCAAGTTAGTGTCTTATCTTGGGTGACTCAAAATCACCGCATCG |
| 2229 | 1 | 2341337 | CCAGCTCAGCATGTAGCAAGGACCACCGAGTGCCAGCCTGGTTCCCTACG |
| 2230 | 16 | 10912331 | CTGGGCGCCAGGTGGGCTCCTGGGAGGGTGATGAAGCAGGTAGGGCCTCG |
| 2231 | 4 | 85424469 | CGTACCTGGGAGTTAAGTGCGCTCCGAAGGGTGGCTTCTGAAAGGTCCAA |
| 2232 | 1 | 45792660 | CTCCTGGCTGGGGTGGCGCTGCAGGGAGAACCGCGAGCTCTCAGGGGTCG |
| 2233 | 11 | 111383576 | CGCCCTACCATGGCTGGGCGCAAGCGGCCCCCGAGGACACCCCCGGGCCC |
| 2234 | 15 | 45404157 | CGGGCATGGGGCGCCAATACGTGACAAACCGTCCGTATTGCGCCCTCCCC |
| 2235 | 14 | 92413706 | CGCTCGGCCCCTCTGGAATAAAACACCCGCGAGCCCCGAGGGCCCAGAGG |
| 2236 | 3 | 170303721 | CGTCGCCTCCAGCCCCGACCCCGGTAAGTGAACGGCCCCCTTCGCTGCCG |
| 2237 | X | 136509643 | AACCTTGGAGACCCTGGCCCGCACGCGGAGTGGCGTAGTGCTAGCTACCG |
| 2238 | 15 | 68260574 | CGCGAGTTCCACCAGTCGGGCTGCGTTTTGTGAACATGGAAACCAGATTT |
| 2239 | 21 | 15077562 | CGGCCCTCCCTAGCCTGGCGCCTCAAGCTCGCCGCCCGGCTTCCCCGTGA |
| 2240 | 6 | 32015635 | CGGCACCACCTGGAGCCGACCATCCTTATCCTTGTACTGGACCACGAAGG |
| 2241 | 10 | 45914525 | ATGGAGCCGCCCTCTCCCCAGGACCACAGGGGCGCTTTCCAGTGAAGGCG |
| 2242 | 4 | 175138377 | ACTTGGACAGAAAGATTCGAGGCAAATATTCTAGGCTGGGAAATTTCGCG |
| 2243 | 18 | 32173084 | CGGAAACACTTGGAAGCTGAACATCAGCCCGAGGACTCACTATGCTTCCC |
| 2244 | X | 124339995 | AAAGCTTCCAACTGTTCCCTTCCCTGGGTGGCGGCAGCTGCCCAAGGTCG |
| 2245 | 17 | 71702722 | CGCCTGTGTGCCCACTCAATAGCAAGCCCAAAGCATGCATCCCAGAACAG |
| 2246 | 5 | 112073398 | CGCACAGCCTGCCTAGCCCTAGCCCCGTACCCCTCGGGGGTATGCCCTCG |
| 2247 | 6 | 27064968 | TTCCCCCTCGAAGCGTTTCCACTCAGTTCTTGATCATTCATGTGAAAACG |
| 2248 | 20 | 49576235 | TCTTACAGTGGCAGCTTGTTGCTCTTTGATGCCCTGAGAGGGCATTTCCG |
| 2249 | 18 | 5630348 | AAGGAAGGCTTCGGTGACTCAGGTGAGCGCCTTTCTCTTCTTGGCAGCCG |
| 2250 | 11 | 124055251 | ACATCAGTCTTTCCCCCGGGTCTGGATGGATCCATCTGTGATAGATCACG |
| 2251 | 11 | 20185382 | CGGCCAATCTTTCCAAAATGCGCTCTCACTCTGTTGGCTGCTCCGGCAGG |
| 2252 | 11 | 5653405 | TTTTTGGTTTCTCTTCCTCATCTTCTTTGTTCTTTGATATTGCAGTCTCG |
| 2253 | 2 | 170685661 | CGGAGTACTTGTAAGGTTACTGTTACTCCAAAATGTTTGTACCTACTATG |
| 2254 | 2 | 229045417 | GGGGCGGTGGTGCTGGGCCTGCTCCCCCAGCCGGGCAGGTGGCCTTTGCG |
| 2255 | 19 | 15067457 | CGGCCATGTCTTCCATCTCCAGAATCTTCCCAGCAATCAGGAACAGGATG |
| 2256 | X | 13952329 | TAGTAAGGAGCCCACCCTTGGATCTTGGAGAGAGCTCCCTTTTAATATCG |
| 2257 | 17 | 7555298 | CGGCTCTCACTAGCTCCGCAGCCCCGTCTATTTTTAGCTCGTGCCCACCC |
| 2258 | 6 | 26757644 | TTTATGACAAGAAGACCTCTCGGCTTCTTGTAACTGGGGATTCAACCTCG |
| 2259 | 16 | 56484117 | TGGCATAAACTGGCCACTTTCCTCCATCCTGGCATAAAATGGAGGCACCG |
| 2260 | 6 | 50791419 | CCGAGTGGTAGTCCCTCCGGGGGTCAAGGCCCGAGAGCTGGGGCAAGGCG |
| 2261 | 12 | 104852446 | CGCTGAGAAAGGCAACTCGGAGGCGTAGAAAATGGATTTCTGAGCCAACG |
| 2262 | 15 | 97323948 | CGGCCCCCTCCAGGAACCGCCTCCCCCGCCAGCCAAGACTCCATTGACCA |
| 2263 | 17 | 4545157 | CGCCAGGACACCGCCTTTTGGACTGGGGCTGAATTCTGCCCCTTGAAGCT |
| 2264 | 19 | 39694709 | CGGGCGCAGACCTGCCCTACCTGCCCTCCAACTGGGCCAACACCGCCTCC |
| 2265 | 19 | 2294961 | CGAAGGCCAGTGGGAACCGTGAGGAGACGCACAACCTGGTTTGGGCCACG |
| 2266 | 6 | 100917022 | GTTTTTCAACCCGAGTTTCCAATCCAGTGTTCATCTCCAGAGTTAGAACG |
| 2267 | 8 | 131914037 | CGTGCTAAAGATGGCACAGATGGGTTACATCCAGGCAAAACTTGGGCATT |
| 2268 | 12 | 111127346 | CGGGCCACCCCTGCCTCTGGGTCTCCTGATTCCTTGGCCTCGTCCCCTGC |
| 2269 | 8 | 61822740 | CGCCCAGGCCAGCTCAGGGTGTTCCCGTGGGGGTCTCCGGAAACAATGGT |
| 2270 | 2 | 182545492 | CGCCATATGGTCTTCCCGGTCCAGCCAAGAGCCTGGAACCACGTGACCTG |
| 2271 | 17 | 37123638 | CAATATTCTAGAGGCCGCGGGGACGCCTGGTGGGTAGGGCTGCAGCTCCG |
| 2272 | 6 | 32223090 | CGGCCTGGCGGTCCTACTCAACACCGCAAGATTTCAAAAGGGAAATTCCT |
| 2273 | 20 | 25058429 | CGGCCATCACGCTGCTGCCGCCCCAGCGCTTCTCCCGCTTGCGCCATTTG |
| 2274 | 1 | 161697241 | AAGGTCCACGTCGGGTTTCAGGGCTCCAGCCGATTGTTCCAAGGGCGTCG |
| 2275 | 6 | 88877301 | TTGGATCGGGAGAGGATCGCACGGTTTCCACCTGACGGAGCCACGTCACG |
| 2276 | 6 | 110678956 | CCACGGGTCGCATCTTTCAGTGCCCAGATAGACTGAAGTGCGGCCAAGCG |
| 2277 | 1 | 197890608 | AGCTCAGCTGCGGTGGATACTCTCCTTGCAAGAGGGTCTCGAAGTGGGCG |
| 2278 | 15 | 41712591 | TAAAGACCCCCAAGCAGCACTGACAGTGAACTTGTGCTAAAGGTCTTTCG |
| 2279 | 18 | 13611439 | CGCAGCCTGCCCTGAATTTTCTTCACACAGCCTGGGGGCATCCGAATGCA |
| 2280 | 15 | 68121028 | CGCACTCCCGCCTTGGGAACCACAGGGTTCGTTGCTGGGAAGCTGTCTCG |
| 2281 | 6 | 133561756 | GGGCCCCTTCCAGTGCTGCTGGGTGCAGGTATAGGCATCCGTCCCAGACG |
| 2282 | 6 | 100442151 | CGCGAGAGCAGAGATGCCTTCAGATTTTATTCGCGAAGGCACCCCACGCT |
| 2283 | 19 | 1112679 | ACTTCTGGAGTGTGTGTGTGCCAGGTGCGGGCGCGAGTGGCCGTGTGGCG |
| 2284 | 12 | 115136175 | GGCGACATAGCTTCCAGTTAATTAGTCAAGAAATGAATCAAGTCCCAGCG |
| 2285 | 19 | 13617091 | CAAAGAGCAAAGGGCTCCGGGTTACGCTGCGGCGAACGATGCGGAAGACG |
| 2286 | 6 | 85472949 | CGCCGCAGACGCTTATGGCTTAAGGAGGCCCAGGCTCTTGGGACCAGAGT |
| 2287 | 6 | 25962567 | CGGGACAGAGAAGAATAGAAAGCTGACTCAGATGAATTTTGTGATGCCAA |
| 2288 | 6 | 55444091 | CGGCATTTCTGTCGATTCCCGCAGGTGCCCCGTGCACCCGCTGCCCCAGG |
| 2289 | 3 | 129693489 | TTATATCTGCGCCAGGCGGCGGCTCGAGTGAGGTCAGCGGGGAGGGGTCG |
| 2290 | 14 | 85997469 | TGGGGCTTTTTCCAAAACCAAGTCCCTTCCAGCCCTGCTTGTCCTCTTCG |
| 2291 | 1 | 47999163 | CGCGGCGTGCATCACATTATTTCATTTCAAAGAATCCGCAGAGAAACCCG |
| 2292 | 15 | 89949776 | CGGCCGCCCGCACATCTGGCCCCTAGACCTGCTCGGTAGAGGACTGGCTG |
| 2293 | 13 | 25506131 | GGACCGTCGCCAGAAGGCGCCCAGAATTGGGTCCCTCCAGGTGGGGAGCG |
| 2294 | 5 | 32713112 | GGAAACAACAGTCAGAGAAACCCCAAGAGCGGGCGAGCTAAGTGGCGACG |
| 2295 | 17 | 42733542 | CGGGCCAATCCCGAACACATTTACTTTGAGCGGGAAAAGCTCAGAAGCGG |
| 2296 | 21 | 42688540 | CTGTGCCCGGGAGCTCTGAGGTCGCTGGCGGAGGGTGGACCCGTTCCCCG |
| 2297 | 2 | 86038802 | CGCCGAGCCTCAGCTCACACACTCCGCAGACCCTCCTGCGCCCACACCCG |
| 2298 | 7 | 153584416 | CGCGCAGTCAGAGCTGCCTCTCGCCCTCGCTAGCTGGGCTCGCAGCCTCT |
| 2299 | 2 | 37571677 | CGGCCCTGGGTGACAGACACAAGCTTCTGAACCCAAGCTAACAAAGGCGG |
| 2300 | 10 | 102748988 | CGGGGTTGCTGGTCTCCTGGTCGCACCAAGAAGCATCGTTTGGGGTTCAG |
| 2301 | 11 | 30037875 | GGGGGGCCAACAGTTGTTATTTTCCCAAACCCGGGGATGCGGTGAGTGCG |
| 2302 | X | 119133852 | CGCGGCCTGAGAAAGGTGGCACAGCAGCTTCCCGCCCACGCGCCTCTCTC |
| 2303 | 1 | 75595970 | GCGACCAAGAGTCTGTACGAGTGACTCGGTGTTTCTTTCCAGCCGGTTCG |
| 2304 | 1 | 50883329 | CGCCCTGACTGAAGGGCCCGCAGCGGAAAACGCTGCTGCAGAGCAGCACT |
| 2305 | 15 | 60288082 | CGCGGGGAAGCGGACGCGAAGGAAAACCTAAGTTCCTAACAACTATGGAA |
| 2306 | 5 | 140624888 | CGCATGGTGGCGCTGCAGGCTAAGGCTTCAAAAAAAGGAAAAGGAAAAAG |
| 2307 | 3 | 62305147 | TATCTGTAATTTAGACCCCGGAGGGCGGGCCTGTGAGCGATTGCTGTCCG |
| 2308 | 15 | 27787026 | TACAAATACAGAGGGCGGCCCAGCCACGAGGCCCAGCTGAGGGCACGGCG |
| 2309 | 13 | 24902376 | GTGCCCAAGGGCCACAAGAGTATGACGGGGCTGTACGAGCTGCTGTGACG |
| 2310 | 6 | 100903817 | CGGCCTCATTAGTATGCGCCTACGAGGGAAAATGAGACTTCCCCACAGGT |
| 2311 | 13 | 111935508 | GTAGGCGGGCGTCAGCGGCGCGGGCTTGCTGTCTGCGTGCTTGCTGGACG |
| 2312 | 7 | 156400281 | CGCGTGGCCCCCACCCACTCAGCTCCCAGCGCGGAGTCCCGGGACCACCT |
| 2313 | 2 | 102803407 | TCAAAGACTTTCAAGCCACTTCCTAGTGTGATGTCACATTCCTTCCTACG |
| 2314 | 9 | 23822666 | CGAGAGCACCTGTAGTTGGGATTTTTTTCATTGCAAAGGCATCGATTTTA |
| 2315 | 13 | 102572229 | AGCCAGAATAGATGCCCGGAGACTCCGGAGTCCGCTGTTCAGGCGCTGCG |
| 2316 | 6 | 72298424 | CCAGAGGCTAAAAAGCTCAGCTGGAAAGCTACAGTGGGGCGGTGCCAGCG |
| 2317 | X | 136510760 | CTGGGCACTTAGTGTCCAGTTTCCACCCAGAACAAGGTGGCATTCTGGCG |
| 2318 | 8 | 38965026 | TCCAGCAACACAAAGCGAAAAGTTAGTGGAGGACTACGAGCGCGATCTCG |
| 2319 | 1 | 113286757 | CTTGCGAGAAGACCCCTCCCAGCCCAACCAGGGTTAGGGACGGGTGACCG |
| 2320 | 6 | 99277015 | AAGCTCTTGCTAGAGCTCCTGGCTTCTCTCATCCTCTGGAGCAATTTTCG |
| 2321 | 6 | 133562492 | CGCGCAAAACTTCCATCCTGTCCACGTGAAGTTGTCGCTGCCTTAGAGAG |
| 2322 | 4 | 13532951 | AAGCATTTGAATGGACCTCAGCCCTAGGACCCTGGCGCCCTCGGAAACCG |
| 2323 | 17 | 29886890 | CGCCCCGCCCGACCTCGGGTCGCGGGCTCCAAACCCAGCCCTCCAAGGGT |
| 2324 | 4 | 81107087 | TAATGGAGGGTCGGGCCAATAAATTAGCTTTAGGAACAGACGGACGGACG |
| 2325 | 16 | 22959875 | CTCAGGCATGCCTGGCCCAAAGCAGGGGTGCAGGCAGCTTAGGGATGACG |
| 2326 | X | 153199588 | TTGTGTGATGGCTCATGACCTCCGGGCTGAGTGGCTTGGTCTCCCCCTCG |
| 2327 | 2 | 182544321 | CGCCCTGCAGCTAGCGCTTTTGTACAAGGTGGAAATTTAGCGGTGGGCCA |
| 2328 | 7 | 137028410 | CGGGGAACCTGATCCTGCCTTTGACTCAATTACGCCCTGACAGGGAGGGA |
| 2329 | 3 | 42543530 | GTCACCCGGGAGCAGGGAACACCAAGGAACCCAGAGTCGGGCCTCTTTCG |
| 2330 | 3 | 149374535 | CTCCCTGCTTCCAGCTCTCCATATCTGTGAAATAACCGTGGAAGGACACG |
| 2331 | 5 | 140306231 | CCGGGCTTGCTAGGCTGGAGGCTGGGCAGCCGTCTGGGAAAGCGGAGCCG |
| 2332 | 6 | 133824178 | CGGCACTGGGAGGGCTGGTCATTTGTTTTTCAAGCTGTGGAGATTCCACA |
| 2333 | 13 | 97646811 | GCTGGTTTTTTGGAACCTCTGCAAACACTTTCTCCTGGGTCAGTTTCCCG |
| 2334 | 13 | 113345944 | GAGGCTGGCGGTCACTGGTCAATCACAGGTTCGAGAAGTCGCATGCACCG |
| 2335 | 1 | 62660624 | CGCCGGGCCCCATGCACCCGGGCCAGCCTTACCTGGCGAAAAAGGAATCC |
| 2336 | 11 | 131609432 | TCCACAGGAACCGCGGAGGACAGAGCATGCACATAGGTATGCCAACGGCG |
| 2337 | 3 | 16216094 | GAGTTGTGCAAAGCCTGGGAACAGTGAGGGCATTAGGACCAGGAGTGACG |
| 2338 | 7 | 8481460 | CGCGCTCATCAGAAGTTTAAACAACAGCCACCAACCCGCGAGGGCAGGGC |
| 2339 | 18 | 59000860 | CTACCCCCTCTGTCAGAAGTGTCACAGCTTTTCTGCCTTCGGATGTTTCG |
| 2340 | 6 | 136173085 | CGGTGCTGCTTGTTCATGAAGACTTGTGCTGCAGAATCCAGCAACTTCAT |
| 2341 | 6 | 146136563 | GGTACACTGCAAAAGCGGCTTCAAGTCCCCTCCCCAGCTAATTACCTTCG |
| 2342 | 1 | 50883394 | CGCTCTGTTCCTGCTTCAGTGCGCACCTTGACGGCTCCTTAGGGACAGAC |
| 2343 | 16 | 23313293 | CTGTACTGGTGAGCGCGATGCTTCGCCTAGGTGAACGGGACAGGTACACG |
| 2344 | 15 | 65360058 | CGGCGCCACTCACCAGACTTGCCAGCCCCGCGGCGCCCCAGGATGGCCAG |
| 2345 | 13 | 20806732 | CCGTGGCGGGGGCGCAGGGTGGGGGACCCCTAAGAAGTGCACAGTGCGCG |
| 2346 | 3 | 44063593 | CGCAGCCCGGTGACCACCCCGAGGAGCGCGGCTCCGTTACTTACCCTGTC |
| 2347 | 8 | 121137734 | GACTTTCTTTTCACGCGCGCGCCCAGAATGGACTGGGGACCTTTGGGACG |
| 2348 | 2 | 162283189 | CGCTTCTCCACGAGGTAAAGAGTTCTGCGGACTTCACTAGACGCAGCGCC |
| 2349 | 8 | 142247880 | GCTGGAGGTGAAACGGGCAGCCTGAGGCCTCCAGCTCACTTGGAGATACG |
| 2350 | 14 | 24803873 | CGGGGCTGGGGAGATCATGGCCCGCCTCTTCAGCCCCCGGCCGCCCCCCA |
| 2351 | X | 119006837 | CGCAGTAAATTCCTAAAGGCTTGCAAGAGAAAGTGGGGAATAAAAAAGTC |
| 2352 | 3 | 147125712 | CGCGCAGTTGTTGAATAGACTTTGCGCTAGGCAAAGACAGGTTAATCGAG |
| 2353 | 14 | 29235123 | CTGGGCACCTCTCATTTCGGAAGGCCATCAGAGGCGCCCACTACTGAGCG |
| 2354 | 7 | 152623043 | CGGGCAGCCCCACAGTGGGCGATTTTCCCGCACTCCGCACGAGTCCATCC |
| 2355 | 19 | 15162821 | CGTGAAATTGTCATTCTAGGGGTGATATATTACTACTTCATGGATTATCA |
| 2356 | 14 | 42074635 | CGGAGCTGCGCTCCTCTGGAACAAAGCAGCCAGACGTGGCTTGAGTGGAT |
| 2357 | 19 | 2250956 | GGGCGGCGGGAAGGGCACGGTGTCCAGCTGGCCGTGCGCAGGCAGCGGCG |
| 2358 | 6 | 10887587 | TGCGGGGAGACCAGGCAGAATAGAAACCTCGGATCCCTAGCTAAATAGCG |
| 2359 | 1 | 228653136 | CTAACTCCGTCAGAGCGCAGGCTTATAGAAGGCACTGTCTCCGAGCTGCG |
| 2360 | 5 | 140811102 | CGCAGCTGCTTGTAGTCAACGCTACCGACCCTGACGAAGGAGTCAATGCG |
| 2361 | 14 | 91580613 | CGCCCCAAACAAACAGTAAGTCAGAAGGGAACAGAGCACAGCACAGTCCC |
| 2362 | X | 108868239 | GCAGCTCGAGCAACAGGCGGCTCAGAAGGGTTCGCAGCCGCTGGCTCTGG |
| 2363 | 11 | 131640135 | CGGGGACTTCTGGTCTCCTCTGTCAGGGGACAGGCAAATCGAGTGACTAG |
| 2364 | 5 | 35991215 | ACAATATCTACACTGGGTGAGTGCTTGGCCGGAGAATTCCCAGACAGGCG |
| 2365 | 12 | 66123127 | CTGGGCGCGCAGGAGGTATAGGACTCTGGGATTTTTTGGCCAAAACTCCG |
| 2366 | 8 | 26687962 | CGAACAAGGGAGTCTTGATTAGTACATTTGGGATTCTGGAGATGGCAGAA |
| 2367 | 5 | 169064530 | CGGCTTTCCCGCCCCGAGGCCCCGCCGCAGGCTGCGCCCTCTGACCTGCA |
| 2368 | 12 | 56415271 | CGCAGACATGCATACACCACCCGCACTCCCTCGCCGTTTCCAAGGCGGCG |
| 2369 | 12 | 115136287 | CGCCTAGGCGTCCAAGCTGTGTTCCCCACCGCGCCCCACTCCGCCCAAGG |
| 2370 | 6 | 117086601 | CGGGGCCCCCAGCAATTCTGGGCCCCAAGGTCACGGTGAGCCAGGGCTCC |
| 2371 | 4 | 114214449 | CTTCCAAAACAAGGCACAGCTTAGGAAAATCACTAGTTTTTACCATTACG |
| 2372 | 2 | 66664960 | CGGGTACTGATGCGAGTGCAGAGGAGGCCCGTGGTTCAGGTGGTGGACCG |
| 2373 | 7 | 101006035 | CGCCACTGCCGCCTCCTCGGCCCCGGACCGCCGAGGGTTAACAAAAAAGC |
| 2374 | 6 | 78173227 | CGGAACTATCAACTGGGGACAAACCTGCGATCGCCACGGTCCTTCCGCCC |
| 2375 | 14 | 26674085 | CCCTGAAGCCGCATTGGTGGGTCTCCATTTAGTTTGTCCTTTAAAGTCCG |
| 2376 | 5 | 72740693 | CGCATCTGAAATTTAGATCTGAAAATAATTACGAAGAGAGCCAAGAACAA |
| 2377 | 3 | 147125942 | GTCACCCTTGTCACAGTTATTGAAATTCCGCAACTCGCCACACCAGGACG |
| 2378 | 5 | 12669584 | CGCTTTGGTGGAACAAGTGATTACAGGAACAGTTCCGGCAATTTAGAGAC |
| 2379 | X | 25041134 | CGCCCCTTGTCAAATCGCCTCCAGGGTCTCGGGGACCCGGTGAACAAAGC |
| 2380 | 2 | 188419087 | CGCAATCTGATCTTACTAGCAGTGAAAGAGCGAGGTAAGAATTTGACTAT |
| 2381 | 6 | 31938533 | GACCCCTCTGGGGAGGTTAACACCAACGTGGCCTTCTGCTCTGTGCTACG |
| 2382 | 5 | 72530472 | ACAATACTTTTTAACCCGATGGTTACCCCTCTGACTCCGATGACCTTTCG |
| 2383 | 7 | 1121738 | CGCAGCTTTCAGACCTGCAGTCCATGTTAAAAGGATCTTGTTTCCAATTT |
| 2384 | 19 | 48565189 | TTGGGGCCGGTCCTGGGTGGGAGGTGGGCTGCGGCGAAGCTGACTTTGCG |
| 2385 | 10 | 118034031 | CGCTGCAGCCGAGGGGCGCGCCTGGCTGATGTGTGGTTGAATGGAGAGCG |
| 2386 | 7 | 19184555 | CGCCTGGCCATCGTCTATATCTCCTTCATGACCGAGCTCTTGGAGAGCTG |
| 2387 | 7 | 5184155 | CGCCGTTCCTCCCACACGTGTCTGAGGTGTCAGTCGCCATGGGAACAGCC |
| 2388 | 12 | 103359836 | CGGCTGAGCAGAGCTCCAATCTAGAAGTGTCCGGGGTTTTGTTAATTGTA |
| 2389 | 20 | 44313578 | CGCTTCTCACAGACCTTGATTCCTGAAATGATGCAGGAGCAGGGTCAATG |
| 2390 | 10 | 79470966 | GGTCCTTTCGGGGACTGCGGCTGCTTCCCTGTTCGTAGACTCAGCAGCCG |
| 2391 | 12 | 81471754 | CGGCGACTCTAGTGTGGCTTCCAAACTTCTGGGCTCTGGGCTCACTTCGG |
| 2392 | 1 | 70035479 | TTCCCCATCAAGTCATAAAGCGAGCGGGTTGGGCGAGAGGGCACAGCTCG |
| 2393 | 1 | 2345398 | CGCGGGTGCACCCGTGTCTCCTGGACACAGAGGCTGCCCTCTGATTGGGT |
| 2394 | 1 | 2345368 | CGCCCCCGCGCCCCTTTGGAGTTCTGACCCCGCGGGTGCACCCGTGTCTC |
| 2395 | 6 | 167809876 | CCCCAGAAGCCTGTTCCTGAGCTGATTGGGGTTCTGGGCCAGGGGTCTCG |
| 2396 | 3 | 173302427 | CGTTGTTTGATCATTTGTATTTCTTGCCTGATGGTCGGATTTACATGGAG |
| 2397 | 10 | 133871212 | CTCCTTCCTGTGCAGGTATCTCTGCTCTTTCGAGTACAGCAGGGGTGTCG |
| 2398 | 12 | 22094371 | CGCCCGCCCCAAGTCAAAGCTGAGCAGAGCAGGGATGGGGAAGGTCTCCG |
| 2399 | 1 | 110626540 | TTTATTAAATGGTCCACACCTGAAAGTAATTGAGTCTCGGCGTGGGTGCG |
| 2400 | 12 | 40618284 | CAATGTATTCCTAAAAGGGGCCATCTGGGCGGTGTCCTCTTTTCCCAGCG |
| 2401 | 3 | 13974614 | TCGGCCAAGGCCGAGCAGCACGGGAAGGTCGGGGAATCCTGGGAAGCTCG |
| 2402 | 6 | 100895679 | ACACTAAAGATGAACAAAAACCAAGGCTGGCGCTCACTTGCGTTGACTCG |
| 2403 | 11 | 131410868 | CGAACTACAGAAGTATGAATTGACTCAAGGGGGCTGGCAGTGATTTATGT |
| 2404 | 10 | 26223310 | GTGTCGGGACAGCGCAGGGGACTGGCTTGGGGCGGCGGTGGGTAGCGGCG |
| 2405 | 17 | 38821403 | TTTCCCATCCTCCACCTTGGCTAACTGAACCTTATCGATAGGATGAGTCG |
| 2406 | X | 47225852 | CGCCACTCATGGACCAAGTCTCCGCCGATCCGTAACAGCGCTGTGTTCAG |
| 2407 | 18 | 61616526 | AGAGATGTCTGAGCGGCGCCGGGTGTTAGTTCCCAGTGAATGGGGAGGCG |
| 2408 | 12 | 25056243 | CGCGAGCATCCTTGTTTATTAAACAACCTCTAGGTGAATGGCCGGGAAGC |
| 2409 | 2 | 228736357 | TTTTCTTGCTCTCTTATCCCCGGCCGATGGGCTTCGTAGCCTTGGAAACG |
| 2410 | 13 | 102569876 | CGGTCTTAACGTGGTTACCGGTGCACTGAAACTCTCAGCAGCGGTTGGAG |
| 2411 | 2 | 99226761 | CGGTACCCAAATGTCAGCTGACTGTGAATAGGAATATGAAACTGGAAATT |
| 2412 | 12 | 104852439 | CGGGCCACGTTGGCTCAGAAATCCATTTTCTACGCCTCCGAGTTGCCTTT |
| 2413 | 20 | 17299350 | CGCCTATGAATGTCAAAGTTCTGCTAGGTTTCTCTGAAGCCTGCTTCTGC |
| 2414 | 12 | 119457146 | CTAATCAAAGACACGTCAACCATCCCCCGGGTTGAAATAATGGGTGTTCG |
| 2415 | 2 | 202122653 | CTAAATGGAGTCAGTATAAATGCTTTCCAATAAAGCATGTCCAGCGCTCG |
| 2416 | 20 | 21490665 | CAGCAACAAGGGTGGGACGGAATCCGTTCCCCGCCGGAAAGATGGATGCG |
| 2417 | 5 | 115297488 | GTTGCAGAGCTAAGAAAAACCATCCCGCCAGGCCCCTTCTTGTGGTAACG |
| 2418 | 2 | 27433191 | TTCTGAGAACAGATCTGTAGTTTTCTAAACACCCTACATTGATCCAAACG |
| 2419 | 1 | 75601120 | GGCGAAAGCGGAGCTGTTCTGTTTAAACGCCCTTTGTGTGTGAGGACGCG |
| 2420 | 17 | 37123909 | CGCCCTTGTTAGTTATTCATGAGACGGAGGGCACCATGGGAGCTGGCTCT |
| 2421 | X | 20134719 | GGGGCACGGCGCAGAGCTGAGATAAACGCCCTTTCCCTGGCCCAGGATCG |
| 2422 | 5 | 83678796 | CGCCCAAGAGTCCACAATTTTCTTGTGACTCAAATAGATGGATAGGTGCA |
| 2423 | 11 | 44333192 | TACGTTCTCGCTTAACGATTTTCTCAGCGGTTTAAGGAAGGCTGCTCTCG |
| 2424 | 13 | 70682127 | CGCGGGCATCGAGGATTCTCAGCTTTTGGCGTCCCAGTCCTGCCTCTGCT |
| 2425 | 3 | 181437712 | CTGCTCTAATCCAGAGGGGCCCAATATTCCAGGGATCCCAGATTGCTCCG |
| 2426 | 11 | 111383889 | CGCGGTTTCTCCAGATACAGTTAAACTGTTAGCTCTCTCTAGGAGTCACA |
| 2427 | 16 | 88717646 | CGGGGAGTTCCTGCGCTTCCCCAGGGCTTCCCAAACTTCAGCTCTCAAAC |
| 2428 | 2 | 46526785 | GGAACTTGTGTATTTTACAACTGCGATGCCTATCGGGAATGTGGCAATCG |
| 2429 | X | 49087465 | GTGAAAATCACCAGGAATACGTACTCCACCTGCTCCTGGGGGTGGGACCG |
| 2430 | 18 | 14748250 | CGCCAAGCTCTTGAACACTCCGGCCTCTGTCGGGAGAAATTCTGAGCAGA |
| 2431 | 10 | 133109029 | CGGCCACTTCCGTCGGTTGGAATTCTCTTAAGCATTGCCCTGAGTCTATT |
| 2432 | 14 | 47572921 | AAAGCCCATCTAGGGAATCTCTCATTTAGGATCCATGGTCATATGAATCG |
| 2433 | 1 | 229567626 | CGAGACCACCTACAACAGCATCATGAAGTGTGACATCGACATCAGGAAGG |
| 2434 | 1 | 116381475 | CGGGGCGAGAAGTGCACCCCGCGTCTCCCCGCCCCCGTGAAAACCCAGAT |
| 2435 | 5 | 139047739 | CGCCTCCCAGACCTCTGGGGCCATGAATCATTTATGAGGCAAAAATGAAA |
| 2436 | 7 | 1542769 | CGATTCATTGGCCTGACCCTTTGAGCCCAGAGCAATGAAGTCTCCTGGGG |
| 2437 | X | 110513817 | ACTTTCCACATTTTCCTCATGACTCAGGATCAGTTTCTGACTGCTGGGCG |
| 2438 | 11 | 121971092 | CTATAGAAGTGTAAAGACGAATAGCGTTCTGAACTCACCCATTCGAAGCG |
| 2439 | 1 | 67218275 | CGGAGGTCTGGGAGGCTCCGGGCGAAGCCTCCCTGCTGCAGGTAGGGTCG |
| 2440 | 6 | 100903575 | GGGGCCTACGGAGGCCTTAAACCCCAAAGATTGGCGGGGACGAAGGGCCG |
| 2441 | 13 | 79170283 | CGTGGATTCGACTGTGCCGGGGAGAACCCGAGGTCAACGGCCTTAGGCCG |
| 2442 | 7 | 52156170 | TAGAGGCTCCCCTTCCCCAGTGTGGGACTTAGCAGAGGGCGGGTCCAGCG |
| 2443 | 1 | 2777244 | CGGCAACCATTTGTTCCTGGTGAGCCCCACCAAGGCCGGGCGCCTGGCTC |
| 2444 | 8 | 72756901 | TGAAGCCCGACGGCCCTTCAACATGTCTGCGCAGGATAAGTGAGCTTTCG |
| 2445 | 3 | 13974286 | GGGTCGGGGCGTCCCTGCGCACATTTAAAGGGGCCGTGGCCATGGGCACG |
| 2446 | 6 | 35479648 | ATCCCTAGGGTGAGGAGCCCGAGGGGGTGCGCCCAGGCTTGGGGGTAGCG |
| 2447 | 3 | 170746073 | CGCTTTCCTGCAGAATGTCATGTCACTGGCAAATGCCATGGAACCGCTCC |
| 2448 | 1 | 64937451 | CGGACATGATAAACAATCTGGAGAAATGGGAACCTTATTCACTTTGCTCC |
| 2449 | 4 | 106631934 | CGGGTTTAGCTGAAGGCATTATGACTCCATGGCAACAGATGATTCCCTGA |
| 2450 | X | 153713930 | TTGGAGAAGCCCTTCTCCATTGTCTCAGTCACTTCAGGGTGCAGGAAGCG |
| 2451 | 15 | 72520632 | CGGGCTCTGCCAGTCTACACTTAAGCTCTTAAGGCAGTACAGAGAACCAG |
| 2452 | 10 | 73156377 | CGTAGAGTTGCCACAGCAGCCGGTGCCATGACATCGGCAGAGGGCAGCTT |
| 2453 | 2 | 179914864 | CAGGCACTTAGTGTCACCATACTAGGGGCGTGGCAATTCTCTCTCGTTCG |
| 2454 | 12 | 91775501 | TGTTTGTAGCTCAGGCACCCGGATGATTCATCCCATGAAAGCCTTGAGCG |
| 2455 | 1 | 108023249 | CGAGCTCCTGCACTGCCAGAACGGAGGGACGTGCCACAACAACGTGCGCT |
| 2456 | 7 | 18329602 | CGGATATAGAAACTTCCACAACTGGGGGCCCTGGCTCTGACGACTTTCTT |
| 2457 | 10 | 103600846 | GCCGGGCTGGGAAGACCTGAGGGATTAGACAGGGGTCCGGGCGGGGCTCG |
| 2458 | 11 | 124267100 | GGTCTACATTGTCACCATGGTAGGCAACCTTGGCTTGATCATTCTTTTCG |
| 2459 | 2 | 40739661 | ACATGGTTTGCATAGCTGGATCTCAGCTTATCTGTGCCTTTCGGCTTTCG |
| 2460 | X | 137793853 | CGGGAGGGGGCTGCAAGGAGGCTGTCGCGCTGTTCCGGAGGGTGCCTGCG |
| 2461 | 6 | 53919041 | CAAGAATTTACATCCCACACTGAGTCATGCAGTAAGTTTAGTGCTTCCCG |
| 2462 | 19 | 54466441 | CGCACCAGAGGCCCCGCTCTTCGTTCCAGCGCTTCAGCGACTCCAGTTTG |
| 2463 | 17 | 76834778 | GGGCTGAGTTTGGTTGGGCAGGTGCTACGCGGAGCTCCTGAATGTAAGCG |
| 2464 | 1 | 2345374 | GTCCAGGAGACACGGGTGCACCCGCGGGGTCAGAACTCCAAAGGGGCGCG |
| 2465 | 7 | 29186110 | GTCCTCGTGACTGGGTGGGGCTGCCTCACTTCTGCCTGATTTGGGAAGCG |
| 2466 | 4 | 158143538 | TCGCGAGCTCCATGTTCTCCTCTTTGGGACAAGTTGTTGAAATGGTTCCG |
| 2467 | 6 | 108485102 | TTACAGCCGGGCGCACAGAACACAGGATGCATATGCTGCGGGTGTGTGCG |
| 2468 | 4 | 175139486 | TCAAGGCCATGATTGCATCGCTCTCTGGAACGAAGATGGCCAGGAATTCG |
| 2469 | 20 | 31070190 | AGGAATAATGCTAATTAGCGAGGCTTAATGAGAACTTCCCCCAGGAAGCG |
| 2470 | 12 | 85667353 | TGGAGGGAGCCATGAGCGACTGCCGTGCGGGACGTCTGGAATGAGGACCG |
| 2471 | 13 | 103047225 | GGCCCCGGGCTTCCAGAGAAGTGTGTCATTATTTGTTTTCTGCAAAGCCG |
| 2472 | 10 | 124578209 | CGCACACCGTGCCCCACACGCCCTGGTGCCGCAGCTCTACCCGGCCCTCA |
| 2473 | 2 | 80549634 | ACGTAGACTTAATCCCGAGAGGCAATTGTTCCCTTGCTTGGGCGGCTACG |
| 2474 | 19 | 51522561 | ACGGGAAGATGCCGCGAGGGGCGTCATTAGGGTAATTGTGCCCATTACCG |
| 2475 | 1 | 1005398 | AGTGAGGTGTGTGTGACATATTCATGTGACCACCCGTGCAGCGTCACGCG |
| 2476 | 7 | 100091255 | CGCCCGCGGCACAGCCCCACCCCGCGCTGCCGCTGCCTCTGCCCCTGCCG |
| 2477 | 10 | 50602990 | AATTGAGCTGCCCAGAATCCAGGGCGGAAGAGGCCCCTGCAGTGTTCTCG |
| 2478 | 2 | 201450560 | TCACTGGCGCCCTCTATGTGGGCGGAAATCATCCGGCGAAGATGAAGACG |
| 2479 | 3 | 173303347 | TTACAAGACAACATGTGTGGCCAGCCACCCACTTCTACTGTCTCTATCCG |
| 2480 | 11 | 111847747 | CGGGGCTCCCCAGCACTCAGACCACTAGAGCCCTCCTCGTGATTCTGCCG |
| 2481 | 14 | 39703302 | CGGGAATCCCAAGTGAAGTTTAAGCCAATTGTTCAGGTTGTAGAGATGTC |
| 2482 | 13 | 114780781 | ACATGACGGTCGGGCAGCTCACCCCAGACTCAGTGATGGCGTGGAAGACG |
| 2483 | 3 | 138658554 | CGAGGCGCTCCAGGCCGGGGTCTTCCCGGCTGCTGGCCCCTCTCGCTCCC |
| 2484 | 10 | 134754286 | CGCCGAGTGCAAAAGAGGCTGTTACTCGGAATTCGGCATGAATGTGGCCT |
| 2485 | 6 | 27512849 | TAAGATATTTGGAAACCTGTGTTCTGAGTTCTTCCTTTTCCCCGGGCTCG |
| 2486 | 1 | 13910796 | CGCGCAGCTGCGTCTTTGGGACCTGGCACCGACTAAGCCGCTGCCTCTCC |
| 2487 | 17 | 46659318 | CGAACAGCAACACGCATAATAGTAAAATAAGAGCAGGGGCTGCGCGCGAG |
| 2488 | 10 | 102497666 | CGGGTTTGCGAAGGATGCGGGTTTTACTTAGTCTCGCTGAGCCTGTTCCG |
| 2489 | 3 | 147125782 | CGCTTGTTTTCGCGATGCGGCCCTCGATTAACCTGTCTTTGCCTAGCGCA |
| 2490 | 7 | 123842309 | GACTAACATATCATTTTTCCCTCTCTCCCCAGGGAGTACATGTTGTGCCG |
| 2491 | 4 | 41746947 | CGGCCCTGAAAGATGCCCTATCCATGAGATGCCTTTTCATCTGCAAACTC |
| 2492 | 3 | 72368071 | CCTGGGCCTGGGGGCTTCAAGGAGATGCTCAGATTGACCCTTCCTGGTCG |
| 2493 | 11 | 31845389 | TGACATATTGGGGCCTCGACAGCAAGGTTGGCCTCTGTCCCACAAGGACG |
| 2494 | 3 | 192958832 | CGGTCAAAGCGCCGCGCAACCAGTTATTCCGCCCCCGGCTCCGCACCCGC |
| 2495 | 13 | 38172876 | TGAGCTCTCCAAAGCCCACTGCCAGTTCTCTTCGGGGACTAACTGCAACG |
| 2496 | 4 | 41882163 | CGCCCTGCCGCGGAAACCCTTCCCGTGGCCTCGAACTGGTCCCCGACGAC |
| 2497 | 6 | 29974717 | CAGTTCGTGCGGGTCGACAGTGACGCCGTGAGTCTGAGGATGAAGACGCG |
| 2498 | 14 | 29234671 | GTATACTGAAGCCAGCTCTTGCCAACCCTGAGTGCTGCTGGAAAGCACCG |
| 2499 | 7 | 1775991 | CGGCCCCGGCCCCACACCAGCCCTGGCCTCTTGTCTGGTTTAAATGATCG |
| 2500 | 4 | 122302265 | CGCCGCTTCCCCGCACCCTCAGGGCGAGGGAAGACTTCCCTGGCTCACCA |
| 2501 | 6 | 123317875 | CGGGGTCGCTAAGCAATGCGGCACTCCCTGGCATCCATCAGCCGCGGCTT |
| 2502 | 1 | 14753722 | AGGACCTCAAATGTCTGCAGGTGTCCTCAAGGCTGGGGTTCATGGTGTCG |
| 2503 | 5 | 10333868 | CAGGAGTGGGAGCCGCACGGCTTGGGGAACGCGGGAGATCGCGGATTGCG |
| 2504 | 18 | 60985732 | CGCCCCATCCAGCCGCATCCCGGGACCCGGTCGCCAGGACCTCGCCGCTG |
| 2505 | 18 | 55107755 | CGGCCCTCTCCTGGCAGCAAGCTCCCAGCGGCCAGTCTGAAGCCAATTCT |
| 2506 | 14 | 33402512 | AGCAGGTGCCTCTCCGGGCGAGGCGCTATTGGCCGAGGGCTGGGGCGGCG |
| 2507 | 6 | 117086941 | GAGCCTGTTTCAAACAGAGGCAGGCTGCTTCCTGAGGAACCAGCCCCTCG |
| 2508 | 10 | 102894120 | CGCCGGTTCTGGAACCAGGTTTTGACCTGCGCATCGGTCATTTTGAGCGC |
| 2509 | 8 | 142632451 | CGCAGGCCATGGGGAGCCCCGCTACCGTGAGCACCACCCACGCCGTCCCC |
| 2510 | 5 | 178367827 | GGGAGGTTAGTCCAGGGGAGCGACGCCCGATCGCTGACGAGAGAGGATCG |
| 2511 | 1 | 241520598 | ACAGTCACGTGGGACAGGGGCGTCCGAAGGTGCCGGCTCGGATGGGCTCG |
| 2512 | 8 | 101118525 | ACCTGAGCAGCGGTCACCACAGTAACGAATTCGCTGCCAGTGGAGTCTCG |
| 2513 | 19 | 1083423 | GTCCAAACCAAATTATCTCCCTGTGGTTCCTTGGGCTTCATCCGACAACG |
| 2514 | X | 145078200 | CGCAGAAAGGACACAGTGACATGTAGTGTAAGTGACTGGCACCTTTCTGA |
| 2515 | 4 | 107956405 | ATTGCATCTTGCAACATTCGGAGCGTGGCCCCAGATACAGAGAACAGGCG |
| 2516 | 6 | 84140997 | GTGATCATAAGCCCATGGTGGGCAGAGGCCCTATGGAGCCAAGGGCTCCG |
| 2517 | 1 | 119532056 | CGGTCCTAGCCAAGCTGAAAAAACGTCTCGGATTTCGCGGACAGCGGCCT |
| 2518 | 1 | 91185422 | CGCGCCAAAGGCATTTGATCCAACCCCGCTCCAGCCTACAGTGGCCCGAC |
| 2519 | 12 | 54723931 | ATTTTTATTGCTGCCAGGGACCTCACCGGGGGCAGTGCTGGCACTGCTCG |
| 2520 | 6 | 27526023 | CGATCTCGCCGTGGAGGCATGGATTCCAACCCTCCAGTGACGGTCGTATT |
| 2521 | 5 | 140342795 | TCCAGATGAAAACCTACCTAATCCATCAAACAGTGTCTACTCTAAACACG |
| 2522 | 6 | 142409993 | TTTAATTTAAATTCAGAGTTAGCCGGAGCCATTTCCCCCCTCTAGAAGCG |
| 2523 | 8 | 55370336 | GAGCCCAGCTCCGGCTAGTTTTCCCGGGGGCAGGTGTAGCCTTGGGCGCG |
| 2524 | 19 | 12666317 | GCTTGCCGAGTCCCAAGCAGGCAGGTGAGCTGGAGGGCGGGGATGTCCCG |
| 2525 | 1 | 110627473 | ACCAGCACGTATGTTTTATCAGAATGGCCTTGAGAAAACGAAAGCACACG |
| 2526 | 3 | 62362783 | TGGGGCTGAGGCCTCGGGCTAGGGAAGGCGCCAAGGTGGATTCGGGCCCG |
| 2527 | 15 | 37173638 | ATAGATGTGGACAAGAGAACATCTTTTACATCTCCACAGCTTTCAACTCG |
| 2528 | 17 | 21367368 | CGGATGCGCGCCTTAACAGTTGTCCGTTTAGCAGGAGGTTTTGAGCTCCC |
| 2529 | 1 | 116381079 | AAAGGAATCAGGGTATTTTGCTGGAAGGATGGCTGCCGAGGCCTACCACG |
| 2530 | 15 | 81426347 | CGAGTACCCCTTGCTTGTCGAGGCAACAAAGAGGAGCTTTTAAATGGAAG |
| 2531 | 14 | 60386475 | AGTCTCGCTCTCTCCCATTCATTATCTTTAGCTCTCGGGTCCTGGAAGCG |
| 2532 | 6 | 28367571 | GTGACCCACAAAGGCCGGAAGCGGCCACGGGGGGTCTAAGAACCAGCCCG |
| 2533 | 13 | 37249481 | AGGAGAAGGCACCTAGGAGAAACGTCTTAGGGAGTAGTCCCGCGGGTCCG |
| 2534 | 5 | 2756113 | GGTTTTCAAATGGGCCCCCGAGGCCTGGGAGATCCCCGAGTAGCCACGCG |
| 2535 | 1 | 158799935 | CGACAGGAGTTACCATGTTTCAGGACAGCTTTCTGCCCTGATATGGTTTG |
| 2536 | 2 | 46527098 | ACTGTGGGGACTGCCTGCGAAACGCCTTGGAGGCCGCTGTGTGAGCTGCG |
| 2537 | 3 | 42727489 | CGCCTGTGCCTCTCCAACTGCTTGGCCGTCTTCCGTCTCGGCCTCCTGCT |
| 2538 | X | 64887827 | TTTGCTGATGGCTGAGGGCTTGGCCAGCCACCGGCCCGCCTGGGACGCCG |
| 2539 | 11 | 64875750 | CGCACAGCTCCTCCGCAGGCTCGCCCAGGGCCAGCAGCAGCTCCACGCAC |
| 2540 | 3 | 170746291 | CGGGGCACCCTGATCAGTTTCCTCAGGTTCAGCCAAGGCCTCTGTGGTTC |
| 2541 | 11 | 6501594 | CGGGATCCTAGGGAAGGCAGCCACAATGGAGATCCCTATCCACGGGAACG |
| 2542 | 7 | 19152335 | CGCCACGGAAACCCAAACAGAACTCCGTGGGGAGCGAGGGTCTCACCTGC |
| 2543 | 19 | 36604764 | CGCCTTACCCCAAAAGAACAGGCAAGAGAACTTCACCAATAGGTAAGGGC |
| 2544 | X | 51139247 | AGGATTCACGGGCCTGTAGGGGCTCAGACTTCAAATTGAACCGCGCCTCG |
| 2545 | 2 | 114033360 | TCATTTTAACGTGCGCAGGTCAGCGATGGGCTGAACGTTCCTTAAAATCG |
| 2546 | 10 | 102894148 | AAACCTGGTTCCAGAACCGGCGGACAAAGTGGAGGTGAGCAAGCGGGGCG |
| 2547 | 15 | 97316699 | AGTAGCCACCATTTGGTTAACAGACATGAAAACAAACCTTCTGACACTCG |
| 2548 | 18 | 73628634 | CGCCTCCCCCACCTGCGCGAAGGCGAGGAGGTCCCACCTGCCTCAGCTGT |
| 2549 | 8 | 1048765 | GGAGTGGGTGGCGGCGTCCGTGGGCGGCCGGGAGCCTGGCGTGGGTGGCG |
| 2550 | 1 | 119532195 | CGCCCCCGGGACCCAGCTCTTTCCGGCTTCTTTGCAAAAGGATAGAACCG |
| 2551 | 11 | 44326516 | TGACAGCAGTGTTTCTTGAATAGGGTGACACTTCAGCCAGGGCATTTGCG |
| 2552 | 8 | 93516589 | TATTACCTTAGCCATCTCCTTATTCCATGCTGACTGCTGAAGCCAAATCG |
| 2553 | 18 | 13611400 | CGGCCACCCCGTGATTTTCTCTGAAAACTGCGACAGACTCGCAGCCTGCC |
| 2554 | 3 | 30383960 | AACTTTGGACCAGCCTGTTTCACTCCAAAGCCTTTGGGGCTCAAGCTTCG |
| 2555 | 3 | 155570275 | CGTGCTGAGAAAGGCTGTGACTAAATTAGTAAGATTTTCTTTAATTACTA |
| 2556 | 17 | 19436896 | CGCCAGGACTCACCCCGGCTCTCCACCTCCGCTGGGGGTTTCAGGTTCTG |
| 2557 | 10 | 75005362 | CGGGTTCCACTAACTTTATCTCCTGAGGAATGCCTCAGGAATGCTACAAG |
| 2558 | 1 | 67391835 | GTCTCCTTGCTACTGAGGCTCTCTTTCCTTTGATCCAGATGGTGTGGTCG |
| 2559 | 17 | 75315667 | GGCAGGAGGAACGGCCGTGCCCCAGGCCAGCGTGCTGAGAAAAGGTTCCG |
| 2560 | X | 63446044 | GGGATGGCAACTTGACACCACAAAGTATGGTTCTATTGGCTTAGCCAGCG |
| 2561 | 11 | 134288122 | CGGTTGGTCCTTCACTGAGATCCCAAAGATCGAAGTCCTGGGCCTACTGC |
| 2562 | 15 | 59729553 | TTGTGCTTCCTCTCGGTGGGCAGGATTTCAGCGGAGTTGCGGGGGACACG |
| 2563 | | | |
| Body | | | |
| 2564 | 4 | 22818572 | CGGAATATCCATAGAAAAAAATTCCAAGAATGTTTGGAGAGCAACGCATG |
| 2565 | 15 | 86233236 | CGCTTCCTGGCATGGGGGTCGAATCATGTCGATAAAATGGGCTCGGTTTC |
| 2566 | 13 | 70681804 | CGATGTGAAGCACATTCTGCGACTCCGCTGGAAACTCTTCAGCCACCCGT |
| 2567 | 8 | 133574073 | CGACAGGTGAGAACAATGACAAGGGATCCACACTGTCAGGAATCTCATTA |
| 2568 | X | 151143302 | AAATCTCCGACGACGTGACGGCTGCTGCGGTGGGGAGCCGGGGAGGCTCG |
| 2569 | 14 | 24803925 | GCGGGGTGGTAGCGGCTTTGAGCGGGTGAGAAAAGCTCAGGTGGGGCCCG |
| 2570 | 2 | 17699773 | CGCCCCTCAAGACCTGGCCTTAGCCTAATCTCAGGGTTTGCCCCACCCCG |
| 2571 | 7 | 158114605 | CGGCATCCTTGGGCACGCACAGTAAGTTTGGCAGTTAAACACGTTAGCGG |
| 2572 | 5 | 154027256 | ATTTTAGCACCCTGCAAACAAGTCCTGAGAGGGCAAGCAAGCTGCTGACG |
| 2573 | 18 | 11947571 | CGTGATGCAGTATGGCCCCCGGGAGGCCGCCCCCAGATTCTAGAGGGGCT |
| 2574 | 17 | 77766120 | CGTGGCCCCCGGCCCCTGCCCACCCACCCCCCAGGTCAGCGTCGCAAGAG |
| 2575 | 4 | 8396056 | CGTGGATTTTGCAAAACCGTCATGTTCTCAAAAGGAGACCAACAGCTGAA |
| 2576 | X | 27999728 | CGGGCATCTTACACTGTGAGTAGCTGTGGTTTTTTTCAAGATCGAAAGTT |
| 2577 | 2 | 21022431 | CGGCCCAGAGTTTGACCCCTCCTAGCGAGTTCTTCCCGGCCAGCTGCCCA |
| 2578 | 6 | 43276744 | GGGAGACAAAGGGCTATTTCGCTGTTACTTCAAAGAGCATTAAAGCCTCG |
| 2579 | 3 | 147096130 | CATTCCTTTGTATGCGCGGGGCGCTGCGTGTCTGAGCTGGCGTCCTTGCG |
| 2580 | 19 | 58554527 | CGGGCTGCCCCGCGCAGAGCAGCTCCACCTGTCCCTCACCTACGCTCCCG |
| 2581 | 8 | 77595359 | CTAAAACCTCACCAGAACCGCGCGGAAGTGAGAAGTCGCGGCCAAAGGCG |
| 2582 | 3 | 38035463 | CGTGGAGGATGACATAGCAGTGTTCCTCAAAAAAGTTCCCGTAAGCCCCC |
| 2583 | 17 | 75315567 | GACTCATGCGTGTGTGGTAGGAATCTTCCAGGAAGTCCCCGTCCCGTTCG |
| 2584 | 7 | 19184961 | CGCAGCTCTCCGGATAGGCCGCCATCGCTTCGGCTTGGCCCTGCCTCTCA |
| 2585 | 15 | 74421918 | CCTTTCCTGGTTATGGCCGGTAGCTGTCTCCAGGGCTAACGTGGGCAGCG |
| 2586 | 2 | 177022056 | CGCAAAGGGCTCTCTCCGACTTGGAAAGTGCAGGGATCCCAAGAATATCA |
| 2587 | 7 | 42277807 | CGCCAGGACACCCCCCAAATGCGCGCCCTGGCACTCGTGCTGCGTCCTGT |
| 2588 | X | 130216901 | TCACTCTTATCAGACTCCAAGAATTTAACCAAACCAGACAATCTAATGCG |
| 2589 | X | 151286477 | CAGGGAAGCCCTAGGGGGCCGGATGTGATGCTGCTGACTTGTGCATTGCG |
| 2590 | 6 | 105398579 | TTCGTCCTGATCACTCCATCTAAAAAACTGGTGTTCCCATAAGAATTACG |
| 2591 | 3 | 62363076 | CGCCGCGCCCTGGCCAAGCAACCCGCACACCACTGACTGGGGATCCGGGC |
| 2592 | 1 | 119531093 | CGCCAGTCGTCCCCTGGATGCTCCTGGGCCGACTTTAACCCCTGGCTTCC |
| 2593 | 4 | 189059677 | AACTCCGAGATTGGTAACAAATGCCACAAATTCACTGTGTCCCCTGCTCG |
| 2594 | 4 | 13540946 | GGCGCCCGGGTCCGCAGGGTAGGTGGAGTCTGCGGTGGAAGATGGAGCCG |
| 2595 | 13 | 99947171 | TAGTATTCTTTTATATTTCTTTCATTGGGCACTTTCCCATCTCCAACTCG |
| 2596 | 6 | 108490895 | CTGGAGAGGGCCAGGCAAGGGCACAGCGGGTCTTGTCCTCCACTCCTTCG |
| 2597 | 11 | 111385461 | GCAGGGGCTGGTACCAACTTGGCGGTTCTCGGAGGGGCGGGGCAGGGGCG |
| 2598 | 12 | 85673347 | AATCCGAAGCCCAAGGAACCAGATGCATTGGCAACTGTTAATCGCAGCCG |
| 2599 | 17 | 75279120 | CGGCATCTGTGCAGGAGGGTCTCCGCAGAAAAAACCCAGGACCCACGACC |
| 2600 | 4 | 85417077 | GGTGTCCGCCACAGACTGAGCTTTCTTTCCCCACAAAACATTGTTTTTCG |
| 2601 | 17 | 3792074 | CTCCCGCCTGGATCAAATTCAGGTGACTAAGCGCGTCTGCGAGGGAGGCG |
| 2602 | 2 | 175198280 | CGCACCCTGGTCTGCTTGAGGACGCCTCTTTTCACACTGACAAGGTCCAG |
| 2603 | 7 | 15726166 | CGGATAATCCCGGTCCTGAGCCCCAGCGGCCAGTCTCCTTTACATATGAA |
| 2604 | 11 | 811669 | CGAAAGCCCGTCCACATCAACTCACCCTGGGCAATGACGTCTTCAATGTT |
| 2605 | 7 | 24323840 | CGCTCGCCCGACAGCATAGTACTTGCCGCCCAGCCACGCCCGCGCGCCAG |
| 2606 | X | 53029709 | CGGAGCCTGGGCTTTAAGGACTGGATGACCAGTGGGATCCAGTCTTCAAG |
| 2607 | 11 | 116371487 | CGCGCCCCACCCTCAGCCGCCCCCAGGCAGCCTGCACGACCCAGCTCCTG |
| 2608 | 4 | 13524041 | CACCTGCGGAGGCCTGTGGCGAGTGCCGAGCTGAACCCACACCTGGAACG |
| 2609 | 19 | 41306455 | CGGGGGATGAAGACACTGCTGCCATGGACAGCCCGTGCCAGCCGCAGCCC |
| 2610 | 12 | 133484026 | CGCCCCAGCCCGTCCTGGCTGCCACCCGCCGTCCCTGAGAAACGCTCAGA |
| 2611 | 15 | 86233220 | TATCGACATGATTCGACCCCCATGCCAGGAAGCGTGCTCTCACAGAAGCG |
| 2612 | 4 | 46995743 | TCCCCGGAGTTACAGTCGGAACAAACTTGCCGAGTCTTGCAGCGCAAGCG |
| 2613 | 6 | 121972775 | CCTTATGAACCTGGAGGTATTGTGCTTCCCACATCACAGCAGCAGACACG |
| 2614 | 12 | 81471533 | CGGCCCATCTCTCAATTTTCAATCCAGCAGTTTGTACACGGCTGGTTTAA |
| 2615 | 1 | 23347412 | ACTAGACCGACCATTAAATCTCCAGAAAAGGCCCAAGTCTAAAGTCTGCG |
| 2616 | 14 | 36990534 | CTTAGCTGTTTTAGTCTCCTCTGCGCGCCAGGGGCTGGGAGGGCGGAGCG |
| 2617 | 19 | 2251067 | CTCGAGTTCCGCGGATGGCCTGGAACCCGCAATTGGAGGAGTTGAGAGCG |
| 2618 | 4 | 15780238 | CTTAGTCGCCAACCCACCTCATCTCAGGATGAATTTCAGTGTACTTGACG |
| 2619 | 1 | 159507162 | CGCACACACAAGGGCTAAGGATGTACCAGAGAAGAGCATACGTAATTATG |
| 2620 | 13 | 111039873 | CCCCGACTGCTGGGCTGCCTCCTCGGGCAGGTGTGAGACTGCAGGGGACG |
| 2621 | 1 | 119527377 | GGTACCTCACAGACCCTTCTCCAAAGTCTGGCGGGCCTTGGGGTTTTTCG |
| 2622 | 7 | 27153944 | CGAAGTTTCTCAAACGAATTCCTAAGAAATAGAGCCCAGCAAGAGAGCCC |
| 2623 | 20 | 44640353 | CGGGACAAGCTCTTCGGCTTCTGCCCGACCCGAGGTACCTCCACCCTGTC |
| 2624 | 2 | 239079168 | CCGACAACGTCACTGTGATGGTGGTGCGGATAGGGCACTGAGGGGTGGCG |
| 2625 | 6 | 21667166 | CCTCTCAATTTCTGCATATTCATCCCTACATGAGTGCAGCTGGAAGAACG |
| 2626 | 17 | 77765514 | GGGTCTCAAGACAGCTACGAGGGGCTGGCACCGGGAGGCTTTGGCTAACG |
| 2627 | 6 | 74024572 | GGACCCAAGTTCAAGTTCCCTCCTCTCCAAACTCATCTGTTCTGGTCCCG |
| 2628 | 15 | 90357521 | TCTCTGGCCAACACTGGGCTCAGAGGCCCTTTCTTTAACCTTAGATCTCG |
| 2629 | 20 | 19915716 | TCCGAGGCAATTGTGTCAATGAGCTATTCAAAGACAAAAATAGTATGACG |
| 2630 | 6 | 10887185 | CGCCCCCTGAAGTTTACTAGAGCTCTGTGTTTTGAGACTCAAGAAGCGCG |
| 2631 | 14 | 101520662 | CGTGGTGGATTCGCTTTACTTATGACGAATCATTCACGGACAACACTTTT |
| 2632 | 1 | 158900549 | CGACACCAACTGGCAGATGATGTTGTGTTAACCCAGCAGCAGATCTGCAA |
| 2633 | 11 | 131319616 | CGGCCTCCATGCTTCTGGACACACACACGCTTCTCTGAAGGGCCAGCTGG |
| 2634 | 1 | 203598623 | CGCGCTAAATAACGACCTCACGGAGAAGCCGTCCCCGAGCCCACCAACAT |
| 2635 | 11 | 31846533 | CGGGATCCCCGGAGCCTGCCGTCCTTTGTTGAAATTACCCTCTGCCCAAT |
| 2636 | 1 | 64937345 | CGCAAACTACAGCAGGATATGCGGAAAGCAACAAAAAGAGTCCCCCAGCA |
| 2637 | 3 | 26666060 | TTGGAGGAGAGCGCAGGAGCGGAGGCTGCGGTGGGAGCCACTTTCCCCCG |
| 2638 | 12 | 81471884 | CGCGGTCCCCGAGTGTGCGACAAATGGTACTCTTGCAACTTCCTGGGGCC |
| 2639 | 14 | 54507223 | CGTGAGGTGACCCTCACCCAAGCCTTTCATGTGTTTGGCATGCTTAATTT |
| 2640 | 19 | 58554479 | GCTAACACTGCCTGAGGCACCTCTGCTTTTGAAGTCGCCGGGGATTAGCG |
| 2641 | 4 | 9783192 | CGCTGAGGGCGGTGAGCGAGGGCGCCAAGGCACAGGTGGGGCGGGAGTCG |
| 2642 | 6 | 85478807 | CGGGCTCAGACCTGATTCTGATTCTTCTACCTGAGAACTGCGGAGTTGTT |
| 2643 | X | 13588301 | CGCAGCCAGGTGGGGCACAGCTGATGCAAAAGGAGCTAACTAGGTGCAAA |
| 2644 | 6 | 28129616 | GCTGGTTGCTGCTCACAGGCAGAGAAACGCTGGTATGCATTTGGGTGTCG |
| 2645 | 1 | 58716257 | TATACATCACTACCGCATTCCCCCGAATAGGGGAGCCCCTCAGCATTTCG |
| 2646 | 14 | 93897195 | GCCCGTGTCGCTACCATTCGGACAAAACGCACATCAAATTGCAGAATTCG |
| 2647 | X | 130926603 | CGAAACCCTACGCCATTACAGAGTTCTAGTGTGGGCTACCTGTTGGGACA |
| 2648 | 1 | 9377984 | CGCGGAGCGAATGGTGTGCCAAGGAAGCAAGAAACTCCCACCAAAGCGAC |
| 2649 | X | 113817409 | CGGAGGTAGCTGGACCGAAGCAAAAGATCGAGCCTCCGGTTTGGCTACTT |
| 2650 | 2 | 131101463 | CGCAGAGCACAGCGCTCCAGGTGCGCACTCGGGGCCAGCACTGTTCCCCA |
| 2651 | 8 | 38964993 | CGCTTTGTGTTGCTGGACCTTAAGAACTGACACACTTATTCACAGTCAGC |
| 2652 | 20 | 19915762 | GACGTCATACTATTTTTGTCTTTGAATAGCTCATTGACACAATTGCCTCG |
| 2653 | 3 | 147087237 | CGGCTCCCAGTGCGCGCGCATACCACATTTCCACAAACGTGTGTTACAAA |
| 2654 | 1 | 221065480 | CGGAAGACCCAGGACCTTCCTATGCTACACCCAAAGCCATTGAGAACGGG |
| 2655 | 12 | 49297822 | CGCGGTCCTCCACTGGGTAGGGGGCGCTCCAAGACAGGGACTCTTCTAGC |
| 2656 | 13 | 74707400 | GTGGAAGTAGTTGGCCCCCCGGCCTCGAGCGGGGTGGATGAATAGCTCCG |
| 2657 | 5 | 6632020 | CGGTCAGAAAGTTGAAGTTCCACAGCCACTGAGTTGGTAGGTACCACAGT |
| 2658 | 15 | 97326893 | AATTTCCTGGTAGAGACAGGAGTTGTCCTGGGCCCCTGACATCCCAGCCG |
| 2659 | 15 | 89952904 | CGGACCCCTGCGGGAAAACCAGACACTTCTGTTTCGTGGCTTTCGGGCTG |
| 2660 | 13 | 58207859 | CGCCTAGGACCCGACACTGCAGCTGTCCGTTCTTGCCAGAGTCCCGGTCA |
| 2661 | 9 | 89560715 | CGGGGCGCCTGGACCCCACTCGCCTCCATCTTGCTGCTGCTGCTTGGGCC |
| 2662 | 6 | 133562087 | AGAGTTCCAGGCAATTCCGGGGGCTGCCCCACAGAAGCAGGTGGGGATCG |
| 2663 | 7 | 45613676 | GTCACCTGGGGAGTCGAAAATACTGAAATCCTGCGAAATTCCCTGTTGCG |
| 2664 | 16 | 1098847 | CGTGCAGGCCCCGTCCACCCTCGGCACTGCCCGCACCCACGGCCCACCTG |
| 2665 | 6 | 100442120 | CGCGAAGGCACCCCACGCTCCTAGAAAAGAGCACGACGCACCCGATGCTC |
| 2666 | 6 | 100909238 | GCCCGGGGAGTCGGCCGGCCAGGGAAGCTAGTCGGCTTGGTGTAGACCCG |
| 2667 | 5 | 169064024 | GGTTTGTTCAGTCCCTAGGCACGGCAATCAGGAAACTCTCTGCACTTGCG |
| 2668 | 5 | 170735347 | CTCGCGGCTCGGCGGGTCTAGGGACTTCGGGTTCCTGAATGTCAAAGGCG |
| 2669 | 7 | 27265522 | CGGGCCTATTCTCTACACACAAGTAGGGGCCGACAGGCTTGAGCGCCCTC |
| 2670 | 8 | 23083353 | CGGTGGAGTTGAGGTGACATTTCACTGGCATGGAGACCTGAAACCAAAAG |
| 2671 | 18 | 32173093 | CGGATATTACGGAAACACTTGGAAGCTGAACATCAGCCCGAGGACTCACT |
| 2672 | 1 | 50888826 | CGCGCCTCCCGGCTCCACTGGCGACGCTGGAGCTCTTGGCCGCTTCCCTT |
| 2673 | 16 | 66638433 | GGTGGGAGCTCCGAAGGGGTGCCGGGGCGGTGATCGGGAGGTCTCCAGCG |
| 2674 | 12 | 95939442 | CGCCAAGGGTCAAGCACAGTGCTTGGTTCACAGAAAGGGGTTCAATATTT |
| 2675 | 12 | 54500619 | GCGGAGGGTGGTGGGCTCCGAGTCCCGGGCGAGGGCGCGGAAGATGCTCG |
| 2676 | 5 | 33892223 | ATTCAGCTGACAGTGTCCGCTTTCGGCGGGGTGTGGAATGGGCCAGAGCG |
| 2677 | 2 | 3625354 | GAGGGGTACACACATTTCTGAAACTACTAAAATACTCACCTCCTGGGACG |
| 2678 | 10 | 102495354 | GGCGGCGGGCGGCTCGGGTTATCTGAGCCGCTTGGTGTTGGGTGGCTGCG |
| 2679 | 5 | 40680670 | ATCAATAAAATGCTCACTGAAAACAAGTTACAAAAGCAATTTGGAGAGCG |
| 2680 | 17 | 80201887 | AGGAACTCTTGAGGCCCTCGGAGAGCAGTTAGGGACCGCAGGCTGCCGCG |
| 2681 | 5 | 172751269 | ACAATGCCTCCGGGCTGCGCGAGGTTGATAAGGGAGCAGCACCGAGCTCG |
| 2682 | 1 | 65731432 | CGGGGAACACGTGTAGCCTCCGGAGACCTAGGGGTCCATCGCTCCTCCCC |
| 2683 | 8 | 67874206 | CGGAGTTCCGCAACTCTCCCCAGACGTCCGGAAGCAAATCGCGTCCCAGG |
| 2684 | 7 | 31376092 | GCTCTCGGCTGCACTTGGGCAAGAGATAGCTGGAGCTATCGCCGCCTGCG |
| 2685 | 2 | 21022877 | CGTCACCTGAGGGGGGCACTTCCGGTCTCGTGCCGCCTTCCCAGGACCCT |
| 2686 | X | 102319088 | GGGAGCTGCAGAGCTACGCAGCCTTCGGTGCAGTCGTCACTCGTGTCTCG |
| 2687 | 2 | 17719375 | CGGGCGCTCCTGTGGTAGAGGGACTGGAACACTGTCCCATGTTGAGTGAA |
| 2688 | 16 | 86320857 | CGCCCCTCGCCAGCGCGCTCGCAGCCCATCGGCTCCTGCCGGGGCCCCTC |
| 2689 | 15 | 92580100 | CTTCCAAATCAAAGTTAACCAAAACAGCTTCAGAAGATGCCCATGGATCG |
| 2690 | 13 | 79171469 | CGCCCCGGCCTCTCCTGATAGCACCTGGCACCTGCGTGAAGTATCCCTAC |
| 2691 | 3 | 13857594 | CGTCAGTTTTTTTGAAAAGCCACTTTAGGAATAGACCAAAGGGGCTGGTG |
| 2692 | 10 | 102996254 | TTTTCAAGTGCCTTAATTGGCTACTCCCCTCCGCGGGGAATTGGGCGCCG |
| 2693 | 15 | 37387577 | CGCGTTCATCCTGCTCCTCGCTCGCCAGCTCCCGTGCGGGGGCTCTAAGC |
| 2694 | 6 | 28832567 | CGGACGTCTTTTGACTGGGTTCAGGTCACACTACTCCCAAAATACGACAC |
| 2695 | 20 | 43945364 | CGGCCTTCTGCAGCGACTGGCGCTGGCTGCGCGCTCCCATCACAATCCCC |
| 2696 | 6 | 100903839 | CGAGGGAAAATGAGACTTCCCCACAGGTTAATAAACAACCTGTCCGGCAC |
| 2697 | 16 | 87682142 | CAGGCCGCGTGAAGTGAGAAACGGTTTGCGGTGTCCTCCGCGGAGTGCCG |
| 2698 | 2 | 31637348 | TGTCCCCTCTTACTTGTTCTGTCTTTCTCTCTCTCGCTTTAAAGGTGTCG |
| 2699 | 7 | 121950506 | CGGTTCTTGCTCACGAGTTCCAAACAGTTAAAGTGACGTTGGCAAGGCCG |
| 2700 | 3 | 159756793 | CGGGGCTCCAGGCTGGTGCTTGTGCTGAGAATGACTGCGGGAGGGGCTCG |
| 2701 | 19 | 5625046 | GCTGCTGTGATCTTCCTGTCAAGCTGGTGACTGAGACTTCCCATCACTCG |
| 2702 | 2 | 202098951 | TCTAGACACCAGTGGGCCGAGTGGAGCAGATGCCAGTTGGGACCTTCCCG |
| 2703 | 4 | 172734266 | CGCCTGCAGCCGGCGACTTTTCCCCAGGAATCTCTGGCCTGGCGCCCTTT |
| 2704 | 18 | 21199691 | CGGGAAGGCACTCCCTGACCGCAGATCACACTCTGCAGCTGATCCACACC |
| 2705 | 16 | 1362626 | GGGCCTTGTCTCCCTAACGAGTGCCAGGTGAGGCTGCGGCGGCTCCGACG |
| 2706 | 16 | 13283794 | CGCAGAGGGAGGAGAAGAGAAGGGGCAGAAACTAAACAGCCATAGTAGGC |
| 2707 | 5 | 145722201 | CTGGGGGAGATCCGCAAGTTAGCTTATGCGGTAGGGGGTGTCGCACTGCG |
| 2708 | 1 | 43149980 | TGGTAACGTGCAGTAGACGCACTGTATTCCATGGGCTCCCTTGTAAGCCG |
| 2709 | 11 | 32454975 | CCTGGTTAGGCTAAGGTAGGAGCGGCCTGAAGACGCGCGTTTAGAAGGCG |
| 2710 | 17 | 79668668 | GGCAGTTAGAGACGAGGCGTTTCAGGTGAACTGTATGTAGTGTTACTCCG |
| 2711 | 6 | 137242062 | CGCCTGAAAACCAAACTGTCTGGCGCTTAACGGTTACCTCGGCTGAGCGA |
| 2712 | 3 | 126192611 | CAGAAACTGGAATGTGTGCCTAGCATGCCCCTAACCTGTATATGCACACG |
| 2713 | 16 | 26156362 | CTTTGCCTCTGTGGACCCACTGTGGAGGCAAGCAGGACATGAGATAATCG |
| 2714 | 1 | 47696260 | CGGACACAGAGACTTGGTCATAAGGCAAACATTAACCCCCAGCGAACACG |
| 2715 | 3 | 62304515 | AGACGGCCTTTCCCTAGGGTTCATAAAGACCCTTTCAAGCCACCCTCCCG |
| 2716 | 1 | 91184770 | ACATTTTACTCCAAATTACCATTTTAAGAACCCTATGAATCTGCGAGGCG |
| 2717 | 6 | 129204510 | GCCGCAGCAGCAGCGGCAGTCACAGGCACATCAGCAAAGAGGTACAGTCG |
| 2718 | 1 | 214153294 | CGCTATGGGTAGGGAAGCGGGGCTGCCTTGACTCTCAGTGCGGGTCCCCG |
| 2719 | 5 | 175792123 | ATATCGGGCGTCTTGCTTCAATTTCCTCCCGAGGTCTTGAGGGATGACCG |
| 2720 | 7 | 3083338 | CGGAGGGCCTGGTCGCCTGGAGGAAGCCGGAGGCCTGCGTGGAGGAGGCG |
| 2721 | 19 | 59085561 | CGAAGACCTAGAGAAAGATGACAGCACGAAGAGAGACAGGGAGAATCCGA |
| 2722 | 6 | 100903561 | CCCTTGGGCGCTCTGGGGCCTACGGAGGCCTTAAACCCCAAAGATTGGCG |
| 2723 | 6 | 100912168 | AGCCAAGGTCCAGATCTGCCAAGGTAGGGAGCTGAAAGGCCCCTGCCTCG |
| 2724 | 7 | 137028389 | TTGAGTCAAAGGCAGGATCAGGTTCCCCGCCTTCCAGTCCAAAAATCCCG |
| 2725 | 19 | 35395973 | GGAGAATGTTCAGGACTGTCGAGGCGCGCTCCGCACCTGGCCTGAAAGCG |
| 2726 | 15 | 89951870 | CGCGCACCCGTGGCAGCACCACAGAGGGAATAAAAGCAGGGTAAACAATT |
| 2727 | 5 | 153859740 | CGGCGGCTCTGCGGGGTCCCAAGCCTCCCTGGCCTGGAGGCATTCTAGAA |
| 2728 | 11 | 57244416 | CGGACACTGGCCTCTTCGATCTCAGCACCGCAGTCAGAGGTGGTGGGGCA |
| 2729 | 3 | 133393454 | GCGCGAGAGTGGGGTGGGTGACGCGCGCCAGCCACGGAGAATGAGGCTCG |
| 2730 | 7 | 153584609 | GAGTTGATTGCTATTGGGATCCGCTGAGTAAAGACACGGGCAGGGGTGCG |
| 2731 | 11 | 20620680 | ATGCAGGACCCATGCCTGCTTCAGTCATCGCAGAGCTGTGGGGCTATGCG |
| 2732 | 16 | 1730561 | CTTCTGCTTGGGGTCCACCAGACGCTGCACATTTGCCACTCATCTGAACG |
| 2733 | 8 | 73163696 | CGGTTCCTTGCAGTCCTCCGTGTCGTCCCGCACATCCTCATAGACAACGC |
| 2734 | 4 | 101111427 | CCGCGAACTCCTGGCAGGTGAGCGAAGCTGCGGCCTTGGTGGGGGCAGCG |
| 2735 | 7 | 39649443 | TGCTGCAGGGTCGCGGTGGGGTTTGCGTCTGAAACGCCGGCGGTGGCTCG |
| 2736 | X | 139593505 | CGGCCAGACCTCTCGACAGATCTCATTTTGTCGCGCCGTCTGGCCGCCAG |
| 2737 | 3 | 62353894 | CGCTCTGGGGCAGGATGGAGGTTGTGTCCTGGTCAGCCAACGGTTCTGTC |
| 2738 | 13 | 113425199 | CGGCAGAACAAGAAGTGGCTGTGTCACTGGATACCTGTACATTTGAATAC |
| 2739 | 2 | 200332025 | TCCCCCTTTTTCGCCACGCGGAAACTGGCCGGTCTGGACAAGGGATTGCG |
| 2740 | 6 | 100050791 | CTAAATGTGCCCAACAGCAAGGAAAATCGAATTGAGTGCGGGCTCATTCG |
| 2741 | 18 | 76746337 | CGGCTCTGACCACACACTCCCGCGCGTCGACGCCATGGCCCACACCTCAA |
| 2742 | 16 | 62070164 | CGCCCGCCCTGCGTCTCCGCACCAAGCCCTGTAGCTCCGAGAGGCATGAA |
| 2743 | 5 | 158488659 | CGGCTCGGAAGCCGAAGAATCGAAACATGGCTCAAGAAGCTACTTGTCAC |
| 2744 | 7 | 124633001 | ACACTGGAATGTTGGCCCAGTGTATGACTCAGGCTAGTTCCCAGCAGTCG |
| 2745 | 12 | 129035348 | CGGAAGCAGGTTCAGGATGACAGAGCCAACCCCATGATTACAGACTGAAT |
| 2746 | 6 | 84140898 | AGGGGCCCGAGCGAAGCCGGTGTCCCCAGCAGGGCAGCCTGCGAGAAGCG |
| 2747 | 4 | 102712216 | CGCCCTGGCCTCAGTGCCCGAGTGCAGTTGTCTAACGATGGCCCGTGGTC |
| 2748 | 6 | 100901804 | CACTTTGAGAGGGATCCCCTATGGTATCCAGTCAAAGGGGCCGGCACTCG |
| 2749 | 17 | 17714293 | CGCACGGCGGCGACACACGGGAGAAGCGAGCATGTTCCCAACATATATAC |
| 2750 | 11 | 131369037 | TCTCGGAGCCTGGAAGACATTATGACCACTAAGCGGGTGTCCATAATTCG |
| 2751 | 1 | 197881774 | CGCCTTCGTTGGGTGGAACGAAGAGGAGTGTGTGTTTGTCTTAAAAATTA |
| 2752 | 7 | 570041 | AGCAACAGTGATTCTTAGAGAAGTAACATTAACACATCACAGAGCATCCG |
| 2753 | 13 | 19585665 | CGCCCCTGGACACCGCCCAGAAAGGACTCACAGCCAAGTGGCAGGCCAGG |
| 2754 | 6 | 100912940 | GGCCAGGGGCGAGGCGCTCATTGGCCAATAGGGCTGAGTGACACGAGTCG |
| 2755 | 1 | 119530702 | CGGGGCAGGCGCTCACAGACTGTGTCCACTGAACTTCATCTTGTTTTTGT |
| 2756 | 1 | 119542629 | CGCCCCCACGGGACTCAGAACCCTGAGGCTCTTCTAGAGACACCAACTTG |
| 2757 | 1 | 221061070 | GTGGTCACCATCCCTCTGTGAGCCTCTGTGCACCTTGGTAGCCTTTTTCG |
| 2758 | 1 | 45285892 | TCTTGAGGGTATCTTTTCGTGGTGCACCATGGGTATGCAAATCACAGGCG |
| 2759 | 15 | 79574893 | ACTCGCTCAGCAACTTAGCAGGGGGCTCCTAGACTCCCCGAGGGTGAGCG |
| 2760 | 12 | 108237150 | CGGGGCCTGCACGGAAGGACAGGGGCGCCTCCCAGACCCACCCTTCCCGC |
| 2761 | 6 | 22363174 | ATCACAAAAGAATTAATGTTTTCACAGCCCTTTGAAGACGTGGTGTCACG |
| 2762 | X | 136630265 | CGGCGCGAGGGGTTTCATTTGTCTCAGTGTTGCAAGTTTACAGGTGCTGA |
| 2763 | 16 | 6533187 | GCAAGAGTTTGCAAAAACATTGGGCGTCTCATTTGGCGAGCGTTTTGGCG |
| 2764 | 11 | 69706527 | AGAATGCCCTGCCGGAGATATAGGATAGCAGTATCCCGTGGGGTACGCCG |
| 2765 | 2 | 162283994 | CGCAGCAGATGGTGGCGGAGCTGCAAGGGAAACGACCTCCCCAATTTTTT |
| 2766 | 4 | 85417612 | CGAAAGCCAAGTGGAGGTTCTCTTGGGAAGCCCGGGGACTGTGGCAGCCG |
| 2767 | 3 | 64008161 | CGAGGGATTTGCACAAGGCTTCATAGTAAGGAGCCATGTCTAACATGCAA |
| 2768 | 13 | 67803251 | CGGCATGTCCGGATGCTCGTTTCCCAGTAAGATTGTATTAGAAGGCAGTT |
| 2769 | X | 23350651 | GTGTGGCAGGGGAATTTCTAGCCCCTTCCAATGTGTCCCTCCCAGGGACG |
| 2770 | 1 | 26797619 | GAGGTCGTCGTCGTCGCCCAGCAGCAAGGGCTGCAAAATAGTAGAACTCG |
| 2771 | 7 | 143042635 | CTCAGGCAGGGCGTGGGTTTCCCTCAGATTCAATGATCCTGCTGGGCTCG |
| 2772 | 12 | 126675667 | AATTCGCTCCTGCAGTCCCTAAACTTGACTGTAACCACCTCTGGTTTTCG |
| 2773 | 3 | 42543161 | CGCCCCTTGGAAGCTCAGCACCAAAGGCAGGCGGCCAGGGTCCTGGCCCC |
| 2774 | 15 | 65360366 | CGGGCGGGGCCACCCCAAGCGCCACCAAATTCCTTGTAAGGGCTGCGGAG |
| 2775 | 1 | 116711075 | CGGGAGCACCCCGCCAGGGGACCGGGAAGGGTCGAGGAAAACTGCTGCCG |
| 2776 | 7 | 101006058 | TAAGCCGTCAAAGGGGGCGTGGGGCTTTTTTGTTAACCCTCGGCGGTCCG |
| 2777 | 1 | 2943891 | GGCAGTTTTTGTGTCTAATCACTGCCATAGATGGAGCTTCTGGGCTCTCG |
| 2778 | 6 | 22298146 | CGGAAAATCAGGAGTCATATGAGGAAGGCTGGGAAATGAATTCTCTTGTA |
| 2779 | 15 | 74425757 | CAGATGCACGCTGGGCGGTGCACAGGGCAGGGCGGGCAGGCGGTACACCG |
| 2780 | 12 | 22094009 | CGGCACTTAGCTTTCGCGATCGCTATTTCTAGAATTAAGGGACGAGGCCC |
| 2781 | 16 | 57125836 | CGCCTAATTTTCCGCACGGATGAGTAGACCCAGGCCCAGTTCGGATCCGT |
| 2782 | 3 | 62360162 | CGCGGTGCTTTAGTGGCTAAAAGTGAAAGAGCTGCCTTGGCAGAATTCAG |
| 2783 | 16 | 55125644 | CGGGCAGGCTGTGAAGCATCCAACACAAACCGAAGCCGCCCGCCTGGCGT |
| 2784 | 14 | 38677218 | TTGGGCGGGCGGGGACGCCAGCTCTTCTAAATCCAGGTGAGGAGCTGCCG |
| 2785 | 11 | 20692113 | CGCTGAGAGTCCTCACAACTGCGTCTCCGGAGGCAGAACGAAGTGGGGAC |
| 2786 | 17 | 16344574 | CGGCAGATTGTGTAGTGGTGAACCTATGGTTTTCTGAAGATATCTCTTAC |
| 2787 | 7 | 145395986 | CGGAAAAAATTACAATGGATAAGGACACAGTAGGCTTTTTGAGTATCCAG |
| 2788 | 7 | 15725322 | CGAGAGCAATGCGTATACAGCCAGCAGCTCTAAGCCCTCCAAAGTCCTAA |
| 2789 | 19 | 21666098 | GAGCTTAGTGTGGGGCTGCCCGGGCTGGTCCCCAGCTCACCGCAGCTGCG |
| 2789 | | | CGAAGGGCCTGAGGGGTCCAACTGCGCATGTGTATTCCTCGGTTTTCCCG |
| 2789 | | | TAGTTTCAGAAGAATGTCCAGGGCCTTTCCACCACCCTGGGGGCAGCACG |
| 2789 | | | TCTGAGTAAGGAAACAGCCTCCAAGCATCAGCAGAGCCCAGATGAGCACG |
| 2789 | | | GTTGGGAAGGACTTAACTTTTCACCGCAGGGCCTGAGTCCTGGCTTTCCG |
| 2789 | | | GAGGGGCTCAGGCTGCGGGAGCGCCGCTGCAGGAGCCTGGGCGGGCTGCG |
| 2789 | | | CGGGGCACAATGATCTGTCCTTGAGGACAAAGAGTGATTCCGGAGAGAGC |
| 2789 | | | CGGGCCTGGCCCAATTACTTTCTTCAGTCTGAGCATGAGGGCTGGAAGAA |
| 2789 | | | CGCTACCCTTGTGCTTGCGTTGCCCACAAGCTTGTAGTCAGAAGAACTCG |
| 2748 | 6 | 100901804 | CACTTTGAGAGGGATCCCCTATGGTATCCAGTCAAAGGGGCCGGCACTCG |
| 2749 | 17 | 17714293 | CGCACGGCGGCGACACACGGGAGAAGCGAGCATGTTCCCAACATATATAC |
| 2750 | 11 | 131369037 | TCTCGGAGCCTGGAAGACATTATGACCACTAAGCGGGTGTCCATAATTCG |
| 2751 | 1 | 197881774 | CGCCTTCGTTGGGTGGAACGAAGAGGAGTGTGTGTTTGTCTTAAAAATTA |
| 2752 | 7 | 570041 | AGCAACAGTGATTCTTAGAGAAGTAACATTAACACATCACAGAGCATCCG |
| 2753 | 13 | 19585665 | CGCCCCTGGACACCGCCCAGAAAGGACTCACAGCCAAGTGGCAGGCCAGG |
| 2754 | 6 | 100912940 | GGCCAGGGGCGAGGCGCTCATTGGCCAATAGGGCTGAGTGACACGAGTCG |
| 2755 | 1 | 119530702 | CGGGGCAGGCGCTCACAGACTGTGTCCACTGAACTTCATCTTGTTTTTGT |
| 2756 | 1 | 119542629 | CGCCCCCACGGGACTCAGAACCCTGAGGCTCTTCTAGAGACACCAACTTG |
| 2757 | 1 | 221061070 | GTGGTCACCATCCCTCTGTGAGCCTCTGTGCACCTTGGTAGCCTTTTTCG |
| 2758 | 1 | 45285892 | TCTTGAGGGTATCTTTTCGTGGTGCACCATGGGTATGCAAATCACAGGCG |
| 2759 | 15 | 79574893 | ACTCGCTCAGCAACTTAGCAGGGGGCTCCTAGACTCCCCGAGGGTGAGCG |
| 2760 | 12 | 108237150 | CGGGGCCTGCACGGAAGGACAGGGGCGCCTCCCAGACCCACCCTTCCCGC |
| 2761 | 6 | 22363174 | ATCACAAAAGAATTAATGTTTTCACAGCCCTTTGAAGACGTGGTGTCACG |
| 2762 | X | 136630265 | CGGCGCGAGGGGTTTCATTTGTCTCAGTGTTGCAAGTTTACAGGTGCTGA |
| 2763 | 16 | 6533187 | GCAAGAGTTTGCAAAAACATTGGGCGTCTCATTTGGCGAGCGTTTTGGCG |
| 2764 | 11 | 69706527 | AGAATGCCCTGCCGGAGATATAGGATAGCAGTATCCCGTGGGGTACGCCG |
| 2765 | 2 | 162283994 | CGCAGCAGATGGTGGCGGAGCTGCAAGGGAAACGACCTCCCCAATTTTTT |
| 2766 | 4 | 85417612 | CGAAAGCCAAGTGGAGGTTCTCTTGGGAAGCCCGGGGACTGTGGCAGCCG |
| 2767 | 3 | 64008161 | CGAGGGATTTGCACAAGGCTTCATAGTAAGGAGCCATGTCTAACATGCAA |
| 2768 | 13 | 67803251 | CGGCATGTCCGGATGCTCGTTTCCCAGTAAGATTGTATTAGAAGGCAGTT |
| 2769 | X | 23350651 | GTGTGGCAGGGGAATTTCTAGCCCCTTCCAATGTGTCCCTCCCAGGGACG |
| 2770 | 1 | 26797619 | GAGGTCGTCGTCGTCGCCCAGCAGCAAGGGCTGCAAAATAGTAGAACTCG |
| 2771 | 7 | 143042635 | CTCAGGCAGGGCGTGGGTTTCCCTCAGATTCAATGATCCTGCTGGGCTCG |
| 2772 | 12 | 126675667 | AATTCGCTCCTGCAGTCCCTAAACTTGACTGTAACCACCTCTGGTTTTCG |
| 2773 | 3 | 42543161 | CGCCCCTTGGAAGCTCAGCACCAAAGGCAGGCGGCCAGGGTCCTGGCCCC |
| 2774 | 15 | 65360366 | CGGGCGGGGCCACCCCAAGCGCCACCAAATTCCTTGTAAGGGCTGCGGAG |
| 2775 | 1 | 116711075 | CGGGAGCACCCCGCCAGGGGACCGGGAAGGGTCGAGGAAAACTGCTGCCG |
| 2776 | 7 | 101006058 | TAAGCCGTCAAAGGGGGCGTGGGGCTTTTTTGTTAACCCTCGGCGGTCCG |
| 2777 | 1 | 2943891 | GGCAGTTTTTGTGTCTAATCACTGCCATAGATGGAGCTTCTGGGCTCTCG |
| 2778 | 6 | 22298146 | CGGAAAATCAGGAGTCATATGAGGAAGGCTGGGAAATGAATTCTCTTGTA |
| 2779 | 15 | 74425757 | CAGATGCACGCTGGGCGGTGCACAGGGCAGGGCGGGCAGGCGGTACACCG |
| 2780 | 12 | 22094009 | CGGCACTTAGCTTTCGCGATCGCTATTTCTAGAATTAAGGGACGAGGCCC |
| 2781 | 16 | 57125836 | CGCCTAATTTTCCGCACGGATGAGTAGACCCAGGCCCAGTTCGGATCCGT |
| 2782 | 3 | 62360162 | CGCGGTGCTTTAGTGGCTAAAAGTGAAAGAGCTGCCTTGGCAGAATTCAG |
| 2783 | 16 | 55125644 | CGGGCAGGCTGTGAAGCATCCAACACAAACCGAAGCCGCCCGCCTGGCGT |
| 2784 | 14 | 38677218 | TTGGGCGGGCGGGGACGCCAGCTCTTCTAAATCCAGGTGAGGAGCTGCCG |
| 2785 | 11 | 20692113 | CGCTGAGAGTCCTCACAACTGCGTCTCCGGAGGCAGAACGAAGTGGGGAC |
| 2786 | 17 | 16344574 | CGGCAGATTGTGTAGTGGTGAACCTATGGTTTTCTGAAGATATCTCTTAC |
| 2787 | 7 | 145395986 | CGGAAAAAATTACAATGGATAAGGACACAGTAGGCTTTTTGAGTATCCAG |
| 2788 | 7 | 15725322 | CGAGAGCAATGCGTATACAGCCAGCAGCTCTAAGCCCTCCAAAGTCCTAA |
| 2789 | 19 | 21666098 | GAGCTTAGTGTGGGGCTGCCCGGGCTGGTCCCCAGCTCACCGCAGCTGCG |
| 2789 | | | CGAAGGGCCTGAGGGGTCCAACTGCGCATGTGTATTCCTCGGTTTTCCCG |
| 2789 | | | TAGTTTCAGAAGAATGTCCAGGGCCTTTCCACCACCCTGGGGGCAGCACG |
| 2789 | | | TCTGAGTAAGGAAACAGCCTCCAAGCATCAGCAGAGCCCAGATGAGCACG |
| 2789 | | | GTTGGGAAGGACTTAACTTTTCACCGCAGGGCCTGAGTCCTGGCTTTCCG |
| 2789 | | | |
| NM_1 | | | |
| 72056 | | | |
| ; NM_ | | | |
| 00023 | | | |
| 8 | | | |
| 2789 | | | CGGGGCACAATGATCTGTCCTTGAGGACAAAGAGTGATTCCGGAGAGAGC |
| 2789 | | | CGGGCCTGGCCCAATTACTTTCTTCAGTCTGAGCATGAGGGCTGGAAGAA |
| 2789 | | | CGCTACCCTTGTGCTTGCGTTGCCCACAAGCTTGTAGTCAGAAGAACTCG |
| 2787 | 7 | 145395986 | CGGAAAAAATTACAATGGATAAGGACACAGTAGGCTTTTTGAGTATCCAG |
| 2788 | 7 | 15725322 | CGAGAGCAATGCGTATACAGCCAGCAGCTCTAAGCCCTCCAAAGTCCTAA |
| 2789 | 19 | 21666098 | GAGCTTAGTGTGGGGCTGCCCGGGCTGGTCCCCAGCTCACCGCAGCTGCG |
| 2789 | | | CGAAGGGCCTGAGGGGTCCAACTGCGCATGTGTATTCCTCGGTTTTCCCG |
| 2789 | | | TAGTTTCAGAAGAATGTCCAGGGCCTTTCCACCACCCTGGGGGCAGCACG |
| 2789 | | | TCTGAGTAAGGAAACAGCCTCCAAGCATCAGCAGAGCCCAGATGAGCACG |
| 2789 | | | GTTGGGAAGGACTTAACTTTTCACCGCAGGGCCTGAGTCCTGGCTTTCCG |
| 2789 | | | GAGGGGCTCAGGCTGCGGGAGCGCCGCTGCAGGAGCCTGGGCGGGCTGCG |
| 2789 | | | CGGGGCACAATGATCTGTCCTTGAGGACAAAGAGTGATTCCGGAGAGAGC |
| 2789 | | | CGGGCCTGGCCCAATTACTTTCTTCAGTCTGAGCATGAGGGCTGGAAGAA |
| 2789 | | | CGCTACCCTTGTGCTTGCGTTGCCCACAAGCTTGTAGTCAGAAGAACTCG |

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

The following references are each relied upon and incorporated herein in their entirety.
1. Prensner, J. R., Rubin, M. A., Wei, J. T. & Chinnaiyan, A. M. Beyond PSA: the next generation of prostate cancer biomarkers. Sci. Transl. Med. 4, 127rv3 (2012).
2. Ahmed, H. U. et al. Focal therapy for localised unifocal and multifocal prostate cancer: a prospective development study. Lancet Oncol. 13, 622-32 (2012).
3. Gilbert, S. M. et al. Quality of life and satisfaction among prostate cancer patients followed in a dedicated survivorship clinic. Cancer 121, 1484-91 (2015).
4. Johnson, L. M., Turkbey, B., Figg, W. D. & Choyke, P. L. Multiparametric MRI in prostate cancer management. Nat. Rev. Clin. Oncol. 11, 346-53 (2014).
5. Gleason, D. F. Histologic grading of prostate cancer: A perspective. Hum. Pathol. 23, 273-279 (1992).
6. Humphrey, P. A. Gleason grading and prognostic factors in carcinoma of the prostate. Mod. Pathol. 17, 292-306 (2004).
7. Tomlins, S. A. et al. Integrative molecular concept modeling of prostate cancer progression. Nat. Genet. 39, 41-51 (2007).
8. Andreoiu, M. & Cheng, L. Multifocal prostate cancer: biologic, prognostic, and therapeutic implications. Hum. Pathol. 41, 781-93 (2010).
9. Barry, M., Perner, S., Demichelis, F. & Rubin, M. A. TMPRSS2-ERG fusion heterogeneity in multifocal prostate cancer: clinical and biologic implications. Urology 70, 630-3 (2007).
10. Boyd, L. K. et al. High-Resolution Genome-Wide Copy-Number Analysis Suggests a Monoclonal Origin of Multifocal Prostate Cancer. Genes Chromosom. cancer 51, 579-589 (2012).
11. Brocks, D. et al. Intratumor DNA methylation heterogeneity reflects clonal evolution in aggressive prostate cancer. Cell Rep. 8, 798-806 (2014).
12. Cooper, C. S. et al. Analysis of the genetic phylogeny of multifocal prostate cancer identifies multiple independent clonal expansions in neoplastic and morphologically normal prostate tissue. Nat. Genet. 47, (2015).
13. Haffner, M. C. et al. Tracking the clonal origin of lethal prostate cancer. J. Clin. Invest. 123, 4918-22 (2013).
14. Konishi, N. et al. Heterogeneous methylation and deletion patterns of the INK4a/ARF locus within prostate carcinomas. Am. J. Pathol. 160, 1207-14 (2002).
15. Kovtun, I. V et al. Lineage relationship of Gleason patterns in Gleason score 7 prostate cancer. Cancer Res. 73, 3275-84 (2013).
16. Lindberg, J. et al. Exome sequencing of prostate cancer supports the hypothesis of independent tumour origins. Eur. Urol. 63, 347-53 (2013).
17. Woodson, K. et al. Heterogeneous gene methylation patterns among pre-invasive and cancerous lesions of the prostate: a histopathologic study of whole mount prostate specimens. Prostate 60, 25-31 (2004).
18. Schmidt, H. et al. Asynchronous growth of prostate cancer is reflected by circulating tumor cells delivered from distinct, even small foci, harboring loss of heterozygosity of the PTEN gene. Cancer Res. 66, 8959-65 (2006).
19. Arora, R. et al. Heterogeneity of Gleason grade in multifocal adenocarcinoma of the prostate. Cancer 100, 2362-6 (2004).
20. Ruijter, E. T., van de Kaa, C. A., Schalken, J. A., Debruyne, F. M. & Ruiter, D. J. Histological grade heterogeneity in multifocal prostate cancer. Biological and clinical implications. J. Pathol. 180, 295-9 (1996).
21. Jones, P. A. & Baylin, S. B. The epigenomics of cancer. Cell 128, 683-92 (2007).
22. Chiam, K., Ricciardelli, C. & Bianco-Miotto, T. Epigenetic biomarkers in prostate cancer: Current and future uses. Cancer Lett. 342, 248-56 (2014).
23. Strand, S., Orntoft, T. & Sorensen, K. Prognostic DNA Methylation Markers for Prostate Cancer. Int. J. Mol. Sci. 15, 16544-16576 (2014).
24. Hemminki, K. Familial risk and familial survival in prostate cancer. World J. Urol. 30, 143-148 (2012).
25. Lindström, L. S. et al. Familial concordance in cancer survival: a Swedish population-based study. Lancet Oncol. 8, 1001-1006 (2007).
26. Al Olama, A. A. et al. A meta-analysis of 87,040 individuals identifies 23 new susceptibility loci for prostate cancer. Nat. Genet. 46, 1103-1109 (2014).
27. Liu, W. et al. Copy number analysis indicates monoclonal origin of lethal metastatic prostate cancer. Nat. Med. 15, 559-65 (2009).
28. Aryee, M. J. et al. DNA Methylation Alterations Exhibit Intraindividual Stability and Interindividual Heterogeneity in Prostate Cancer Metastases. Sci. Transl. Med. 5, 169ra10 (2013).
29. Mazor, T. et al. DNA Methylation and Somatic Mutations Converge on the Cell Cycle and Define Similar Evolutionary Histories in Brain Tumors Article DNA Methylation and Somatic Mutations Converge on the Cell Cycle and Define Similar Evolutionary Histories in Brain. Cancer Cell 28, 307-317 (2015).
30. Sturm, D. et al. Hotspot Mutations in H3F3A and IDH1 Define Distinct Epigenetic and Biological Subgroups of Glioblastoma. Cancer Cell 22, 425-437 (2012).
31. Feber, A. et al. Using high-density DNA methylation arrays to profile copy number alterations. Genome Biol. 15, R30 (2014).
32. Kim, T. et al. Functional genomic analysis of chromosomal aberrations in a compendium of 8000 cancer genomes. Genome Res. 23, 217-227 (2013).
33. Boutros, P. C. et al. Spatial genomic heterogeneity within localized, multifocal prostate cancer. Nat. Genet. 47, 736-745 (2015).
34. Pereira, V., Waxman, D. & Eyre-Walker, A. A problem with the correlation coefficient as a measure of gene expression divergence. Genetics 183, 1597-1600 (2009).
35. Friedman, J., Hastie, T. & Tibshirani, R. Regularization Paths for Generelized Linear Models via Coordinate Descent. J. Stat. Softw. 33, 1-22 (2010).
36. Hambrock, T. et al. Prospective assessment of prostate cancer aggressiveness using 3-T diffusion-weighted magnetic resonance imaging-guided biopsies versus a systematic 10-core transrectal ultrasound prostate biopsy cohort. Eur. Urol. 61, 177-184 (2012).
37. Pokorny, M. R. et al. Prospective study of diagnostic accuracy comparing prostate cancer detection by transrectal ultrasound-guided biopsy versus magnetic resonance (MR) imaging with subsequent mr-guided biopsy in men without previous prostate biopsies. Eur. Urol. 66, 22-29 (2014).
38. Ukimura, O. & Gill, I. S. Targeted prostate biopsies for a histogram of the index lesion. Curr. Opin. Urol. 23, 118-22 (2013).
39. Baco, E. et al. Magnetic Resonance Imaging-Transectal Ultrasound Image-fusion Biopsies Accurately Characterize the Index Tumor: Correlation with Step-sectioned Radical Prostatectomy Specimens in 135 Patients. Eur. Urol. 67, 787-794 (2015).
40. Thompson, I. et al. Guideline for the management of clinically localized prostate cancer: 2007 update. J. Urol. 177, 2106-2131 (2007).
41. Washington, S. L., Bonham, M., Whitson, J. M., Cowan, J. E. & Carroll, P. R. Transrectal ultrasonography-guided biopsy does not reliably identify dominant cancer location in men with low-risk prostate cancer. BJU Int. 110, 50-55 (2012).
42. Campan, M., Weisenberger, D. J., Trinh, B. & Laird, P. W. MethyLight. Methods Mol. Biol. 507, 325-337 (2009).
43. Bibikova, M. et al. High density DNA methylation array with single CpG site resolution. Genomics 98, 288-295 (2011).
44. Triche, T. J., Weisenberger, D. J., Van Den Berg, D., Laird, P. W. & Siegmund, K. D. Low-level processing of Illumina Infinium DNA Methylation BeadArrays. Nucleic Acids Res. 41, 1-11 (2013).
45. Lowe, R. & Rakyan, V. K. Marmal-aid—a database for Infinium HumanMethylation450. BMC Bioinformatics 14, 359 (2013).
46. Morris, T. J. et al. ChAMP: 450 k Chip Analysis Methylation Pipeline. Bioinformatics 30, 428-30 (2014).
47. Desper, R. & Gascuel, O. Fast and Accurate Phylogeny Reconstruction Algorithms Based on the Minimum-Evolution Principle. J. Comput. Biol. 9, 687-705 (2002).
48. Quail D F, Joyce J A: Microenvironmental regulation of tumor progression and metastasis. Nat Med 2013, 19:1423-1437.
49. Damiano R, Di Lorenzo G, Cantiello F, De Sio M, Perdona S, D'Armiento M, Autorino R: Clinicopathologic features of prostate adenocarcinoma incidentally discovered at the time of radical cystectomy: an evidence-based analysis. Eur Urol 2007, 52:648-657.
50. Gakis G, Schilling D, Bedke J, Sievert K D, Stenzl A: Incidental prostate cancer at radical cystoprostatectomy: implications for apex-sparing surgery. BJU Int 2010, 105:468-471.
51. Cancer Genome Atlas Research Network. Electronic address scmo, Cancer Genome Atlas Research N: The Molecular Taxonomy of Primary Prostate Cancer. Cell 2015, 163:1011-1025.
52. Hernandez H G, Tse M Y, Pang S C, Arboleda H, Forero D A: Optimizing methodologies for PCR-based DNA methylation analysis. Biotechniques 2013, 55:181-197.
53. Haldrup C, Mundbjerg K, Vestergaard E M, Lamy P, Wild P, Schulz W A, Arsov C, Visakorpi T, Borre M, Hoyer S, et al: DNA methylation signatures for prediction of biochemical recurrence after radical prostatectomy of clinically localized prostate cancer. J Clin Oncol 2013, 31:3250-3258.
54. Kumar A, Coleman I, Morrissey C, Zhang X, True L D, Gulati R, Etzioni R, Bolouri H, Montgomery B, White T, et al: Substantial interindividual and limited intraindividual genomic diversity among tumors from men with metastatic prostate cancer. Nat Med 2016, 22:369-378.

1. A method of identifying subjects having prostate cancer comprising:
obtaining a sample from a subject;
isolating DNA from the sample;
determining the methylation status of the DNA; and
comparing the methylation status of the DNA to one or more methylated probes selected from the group consisting of cg00697992, cg01272707, cg01819167, cg01906055, cg02160684, cg02560085, cg03195164, cg03456213, cg04634417, cg06024295, cg11748187, cg13300630, cg13944838, cg14399930, cg15132013, cg16469740, cg17004353, cg17032646, cg18315943, cg19550524, cg20399616, cg24517686, cg25961816, cg26447413 and cg27198013,
wherein the methylated probe comprises a sequence region that extends up to 250 base pairs upstream and downstream from the methylated probe, and
wherein the comparison indicates whether the sample is normal, a non-aggressive prostate cancer, or an aggressive prostate cancer.

2. The method of claim 1, wherein the sample is a biopsy sample.

3. The method of claim 2, wherein the biopsy is from a prostate.

4. The method of claim 2, wherein the biopsy is from a pelvic lymph node.

5. The method of claim 1, wherein two or more methylated probes are selected.

6. The method of claim 1, wherein the sample is selected from the group consisting of blood, plasma and urine.

7. The method of claim 1, wherein the sequence region extends up to 100 base pairs upstream and downstream from the methylated probe.

8. The method of claim 1, wherein the sequence region extends 0 base pairs upstream and downstream from the methylated probe.

9. The method of claim 1, wherein five or more methylated probes are selected.

10. The method of claim 1, wherein fifteen or more methylated probes are selected.

11. A method of classifying prostate cancer comprising:
obtaining a sample from a subject;
isolating DNA from the sample;
determining the methylation status of the DNA; and
comparing the methylation status of the DNA to one or more methylated probes selected from the group consisting of cg00697992, cg01272707, cg01819167, cg01906055, cg02160684, cg02560085, cg03195164, cg03456213, cg04634417, cg06024295, cg11748187, cg13300630, cg13944838, cg14399930, cg15132013, cg16469740, cg17004353, cg17032646, cg18315943, cg19550524, cg20399616, cg24517686, cg25961816, cg26447413 and cg27198013,
wherein the methylated probe comprises a sequence region that extends up to 250 base pairs upstream and downstream from the methylated probe, and
wherein the comparison indicates whether the prostate cancer is non-aggressive or aggressive.

12. The method of claim 11, wherein the sample is a biopsy sample.

13. The method of claim 12, wherein the biopsy is from a prostate.

14. The method of claim 12, wherein the biopsy is from a pelvic lymph node.

15. The method of claim 11, wherein two or more methylated probes are selected.

16. The method of claim 11, wherein the sample is selected from the group consisting of blood, plasma and urine.

17. The method of claim 11, wherein the sequence region extends up to 100 base pairs upstream and downstream from the methylated probe.

18. The method of claim 11, wherein the sequence region extends 0 base pairs upstream and downstream from the methylated probe.

19-20. (canceled)

21. A method of identifying subjects having prostate cancer comprising:
obtaining a sample from a subject;
isolating DNA from the sample;
determining the methylation status of the DNA; and
comparing the methylation status of the DNA to one or more methylated probes selected from the group consisting of cg20399616, cg12737160, cg23624008, cg09789590, cg23095615, cg14273822, cg04294888, cg12098872, cg21981270, cg21526205, cg09806262, cg01940855, cg23855505, cg18423852, cg22260952, cg21883802, cg00036011, cg26573704, cg12727795 and cg01404317,
wherein the methylated probe comprises a sequence region that extends up to 250 base pairs upstream and downstream from the methylated probe, and
wherein the comparison indicates whether the sample is normal or prostate cancer.

22-40. (canceled)
